(19) United States
(12) Patent Application Publication
(10) Pub. No.: US 2025/0108108 A1
(43) Pub. Date: Apr. 3, 2025

(54) HYBRID FLU-CORONAVIRUS VACCINE

(71) Applicant: THE REGENTS OF THE UNIVERSITY OF CALIFORNIA, Oakland, CA (US)

(72) Inventor: Lbachir BenMohamed, Irvine, CA (US)

(21) Appl. No.: 18/974,619

(22) Filed: Dec. 9, 2024

Related U.S. Application Data
(63) Continuation-in-part of application No. PCT/US23/68080, filed on Jun. 7, 2023.
Application No. PCT/US23/68080 is a continuation-in-part of application No. 18/046,462, filed on Oct. 13, 2022.
Application No. 18/046,462 is a continuation-in-part of application No. PCT/US2021/027340, filed on Apr. 14, 2021.
Application No. PCT/US23/68080 is a continuation-in-part of application No. 18/046,875, filed on Oct. 14, 2022.
Application No. 18/046,875 is a continuation-in-part of application No. PCT/US21/27355, filed on Apr. 14, 2021.
Application No. PCT/US23/68080 is a continuation-in-part of application No. 18/046,862, filed on Oct. 14, 2022.
Application No. 18/046,862 is a continuation-in-part of application No. PCT/US21/27341, filed on Apr. 14, 2021.
(60) Provisional application No. 63/349,799, filed on Jun. 7, 2022.
(60) Provisional application No. 63/349,904, filed on Jun. 7, 2022.
(60) Provisional application No. 63/451,302, filed on Mar. 10, 2023.
(60) Provisional application No. 63/084,421, filed on Sep. 28, 2020.
(60) Provisional application No. 63/009,907, filed on Apr. 14, 2020.
(60) Provisional application No. 63/084,421, filed on Sep. 28, 2020.
(60) Provisional application No. 63/009,907, filed on Apr. 14, 2020.
(60) Provisional application No. 63/349,799, filed on Jun. 7, 2022.
(60) Provisional application No. 63/349,904, filed on Jun. 7, 2022.
(60) Provisional application No. 63/302,454, filed on Jan. 24, 2022.
(60) Provisional application No. 63/084,421, filed on Sep. 28, 2020.
(60) Provisional application No. 63/084,421, filed on Sep. 28, 2020.
(60) Provisional application No. 63/009,907, filed on Apr. 14, 2020.
(60) Provisional application No. 63/349,799, filed on Jun. 7, 2022.
(60) Provisional application No. 63/349,904, filed on Jun. 7, 2022.
(60) Provisional application No. 63/302,454, filed on Jan. 24, 2022.

(51) Int. Cl. A61K 39/215; A61K 39/00; A61K 39/145

(52) U.S. Cl. CPC A61K 39/215; A61K 39/145; A61K 2039/70

(57) ABSTRACT

A hybrid vaccine composition that prevents infection or reinfection by both influenza and coronaviruses, comprising at least a portion of a Coronavirus spike(S) protein and at least a portion of at least one influenza hemagglutinin (HA) protein. The portion of the coronavirus spike(S) protein is highly conserved among human and animal coronaviruses. The vaccine composition may comprise a Coronavirus protein comprising either: a structural protein, e.g., a Spike protein, a Nucleocapsid protein, or a combination thereof, or a non-structural protein, e.g., NSP2, NSP3, NSP14, or combination thereof; and at least a portion of at least one influenza hemagglutinin (HA) protein.

### CROSS-REFERENCE TO RELATED APPLICATIONS

This application is a continuation-in-part and claims benefit of International Application No. PCT/US2023/68080 filed Jun. 7, 2023, which claims benefit of U.S. Provisional Application No. 63/349,904 filed Jun. 7, 2022, U.S. Provisional Application No. 63/349,799 filed Jun. 7, 2022, U.S. Provisional Application No. 63/451,302 filed Mar. 10, 2023, U.S. application Ser. No. 18/046,862 filed Oct. 14, 2022, U.S. application Ser. No. 18/046,875 filed Oct. 14, 2022, U.S. application Ser. No. 18/046,462 filed Oct. 13, 2022, the specifications of which are incorporated herein in their entirety by reference.

This application is also a continuation-in-part and claims benefit of U.S. application Ser. No. 18/872,496 filed Dec. 6, 2024, which is a 371 of International Application No. PCT/US2023/068093 filed Jun. 7, 2023, which claims benefit of U.S. Provisional Application No. 63/349,904 filed Jun. 7, 2022, U.S. Provisional Application No. 63/349,799 filed Jun. 7, 2022, U.S. Provisional Application No. 63/451,302 filed Mar. 10, 2023, U.S. application Ser. No. 18/046,862 filed Oct. 14, 2022, U.S. application Ser. No. 18/046,875 filed Oct. 14, 2022, U.S. application Ser. No. 18/046,462 filed Oct. 13, 2022, the specifications of which are incorporated herein in their entirety by reference.

This application is also a continuation-in-part and claims benefit of U.S. application Ser. No. 18/601,925 filed Mar. 11, 2024, which is nonprovisional application and claims benefit of U.S. Provisional Application No. 63/451,302 filed Mar. 10, 2023 and U.S. Provisional Application No. 63/626,937 filed Jan. 30, 2024.

U.S. Ser. No. 18/601,925 is also a continuation-in-part and claims benefit of International Application No. PCT/US2023/68080 filed Jun. 7, 2023, which claims benefit of U.S. Provisional Application No. 63/349,799 filed Jun. 7, 2022, the specifications of which are incorporated herein in their entirety by reference.

U.S. Ser. No. 18/601,925 is also a continuation-in-part and claims benefit of U.S. application Ser. No. 18/046,862 filed Oct. 14, 2022, U.S. application Ser. No. 18/046,875 filed Oct. 14, 2022, and U.S. application Ser. No. 18/046,462 filed Oct. 13, 2022.

This application is also a continuation-in-part and claims benefit of PCT/US2024/019443 filed Mar. 11, 2024, which claims benefit of PCT Application No. PCT/US23/68080 filed Jun. 7, 2023, U.S. Provisional Application No. 63/626,937 filed Jan. 30, 2024 and U.S. Provisional Application No. 63/451,302 filed Mar. 10, 2023, the specifications of which are incorporated herein in their entirety by reference.

This application is also a continuation-in-part and claims benefit of U.S. application Ser. No. 18/046,862 filed Oct. 14, 2022, the specifications of which are incorporated herein in their entirety by reference.

U.S. Ser. No. 18/046,862 is a continuation-in-part and claims benefit of PCT Application No. PCT/US2021/027341 filed Apr. 14, 2021, which claims benefit of U.S. Provisional Application No. 63/009,907 filed Apr. 14, 2020, and U.S. Provisional Application No. 63/084,421 filed Sep. 28, 2020, the specifications of which are incorporated herein in their entirety by reference.

U.S. Ser. No. 18/046,862 is also a non-provisional and claims benefit of U.S. Provisional Application No. 63/302,454 filed Jan. 24, 2022, U.S. Provisional Application No. 63/349,799 filed Jun. 7, 2022, and U.S. Provisional Application No. 63/349,904 filed Jun. 7, 2022, the specifications of which are incorporated herein in their entirety by reference.

This application is also a continuation-in-part and claims benefit of U.S. application Ser. No. 18/046,875 filed Oct. 14, 2022, the specifications of which are incorporated herein in their entirety by reference.

U.S. Ser. No. 18/046,875 is a continuation-in-part and claims benefit of PCT Application No. PCT/US2021/027355 filed Apr. 14, 2021, which claims benefit of U.S. Provisional Application No. 63/009,907 filed Apr. 14, 2020, and U.S. Provisional Application No. 63/084,421 filed Sep. 28, 2020, the specifications of which are incorporated herein in their entirety by reference.

U.S. Ser. No. 18/046,875 is also a non-provisional and claims benefit of U.S. Provisional Application No. 63/302,454 filed Jan. 24, 2022, U.S. Provisional Application No. 63/349,799 filed Jun. 7, 2022, and U.S. Provisional Application No. 63/349,904 filed Jun. 7, 2022, the specifications of which are incorporated herein in their entirety by reference

This application is also a continuation-in-part and claims benefit of U.S. application Ser. No. 18/046,462 filed Oct. 13, 2022, the specifications of which are incorporated herein in their entirety by reference.

U.S. Ser. No. 18/046,462 is a continuation-in-part and claims benefit of PCT Application No. PCT/2021/027340 filed Apr. 14, 2021, which claims benefit of U.S. Provisional Application No. 63/009,907 filed Apr. 14, 2020, and U.S. Provisional Application No. 63/084,421 filed Sep. 28, 2020, the specifications of which are incorporated herein in their entirety by reference.

### STATEMENT REGARDING FEDERALLY SPONSORED RESEARCH OR DEVELOPMENT

This invention was made with government support under Grant No. Al158060 awarded by National Institutes of Health. The government has certain rights in the invention.

### REFERENCE TO AN ELECTRONIC SEQUENCE LISTING

The contents of the electronic sequence listing (UCI 22_11 PCT_CIP.xml; Size: 546,405 bytes; and Date of Creation: Dec. 5, 2024) is herein incorporated by reference in its entirety.

### FIELD OF THE INVENTION

The present invention relates to vaccines, for example, viral vaccines, such as those directed to coronaviruses and influenza, e.g., hybrid pan-coronavirus-flu vaccines.

### BACKGROUND OF THE INVENTION

Influenza (flu) is a contagious respiratory illness caused by influenza viruses. There are two main types of influenza (flu) viruses: Types A and B. The influenza A and B viruses that routinely spread in people (human influenza viruses) are responsible for seasonal flu epidemics each year. The seasonal spread is due to a phenomenon known as antigenic drift (i.e., the viruses evolve just enough to evade human immune systems but not enough to develop into completely new versions of the virus). Thus, every year a new influenza vaccine is developed.

Over the last two decades, there have been three deadly human outbreaks of Coronaviruses (CoVs) caused by emerging zoonotic CoVs: SARS-CoV, MERS-CoV, and the latest highly transmissible and deadly SARS-CoV-2, which has caused the current COVID-19 global pandemic. All three deadly CoVs originated from bats, the natural hosts, and were transmitted to humans via various intermediate animal reservoirs (e.g., pangolins, civet cats, and camels). Recently, COVID-19 is expected to become a seasonal illness like most respiratory viruses, e.g., the flu.

Therefore, it would be advantageous to develop a single hybrid influenza-coronavirus vaccine to combat these seasonal respiratory infections. Currently, seasonal flu vaccines are designed to protect against the influenza viruses that research indicates will be most common during the upcoming season. All flu vaccines in the United States are “quadrivalent” vaccines, which means they protect against four different flu viruses: an influenza A (H1N1) virus, an influenza A (H3N2) virus, and two influenza B viruses. However, the present invention features a vaccine composition that utilizes the most conserved regions of various coronaviruses and influenza viruses to allow for broader protection from said viruses.

### BRIEF SUMMARY OF THE INVENTION

It is an objective of the present invention to provide compositions and methods that allow for simultaneous prevention and/or treatment of a coronavirus infection and an influenza infection, as specified in the independent claims. Embodiments of the invention are given in the dependent claims. Embodiments of the present invention can be freely combined with each other if they are not mutually exclusive.

In some embodiments, the present invention features a hybrid Coronavirus-influenza vaccine composition. The hybrid Coronavirus-influenza vaccine composition may comprise, or comprise a sequence encoding, at least a conserved portion of a Coronavirus spike(S) protein and at least a conserved portion of at least one Influenza hemagglutinin (HA) protein. In other embodiments, the hybrid Coronavirus-influenza vaccine composition may comprise, or comprise a sequence encoding, at least a conserved Coronavirus spike(S) protein and at least a conserved Influenza hemagglutinin (HA) protein. In some embodiments, the conserved portion of the Coronavirus spike(S) protein is derived from a full-length spike glycoprotein. In other embodiments, the conserved portion of the Coronavirus spike(S) protein is derived from a partial spike glycoprotein. In certain embodiments, the conserved portion of the Coronavirus spike(S) protein is encoded by one of SEQ ID NOs: 1, 3, 5-7.

In some embodiments, the present invention features a hybrid Coronavirus-Influenza vaccine composition. In some embodiments, the hybrid Coronavirus-Influenza vaccine composition comprises, or comprises a sequence encoding: at least a conserved portion of at least one Coronavirus protein, the Coronavirus protein being selected from: a Spike protein, a Nucleocapsid protein, NSP2, NSP3, NSP14, or a combination thereof and at least one conserved portion of at least one influenza hemagglutinin (HA) protein. In other embodiments, the hybrid Coronavirus-Influenza vaccine composition comprises, or comprises a sequence encoding: at least one conserved Coronavirus protein, the Coronavirus protein being selected from: a Spike protein, a Nucleocapsid protein, NSP2, NSP3, NSP14, or a combination thereof and at least one conserved influenza hemagglutinin (HA) protein.

In other embodiments, the present invention features an Influenza vaccine composition, the composition comprising, or comprising a sequence encoding: at least a conserved portion of at least one influenza hemagglutinin (HA) protein.

In some embodiments, the conserved portion of the Coronavirus spike(S) protein is highly conserved among human and animal Coronaviruses. The conserved portion of the Coronavirus spike(S) protein is derived from one or more of: one or more SARS-CoV-2 human strains or variants in current circulation; one or more coronaviruses that have caused a previous human outbreak; one or more coronaviruses isolated from animals selected from a group consisting of bats, pangolins, civet cats, minks, camels, and other animals receptive to coronaviruses; or one or more coronaviruses that cause the common cold; or one or more coronaviruses that case past, current, and future outbreaks. For example, the one or more SARS-CoV-2 human variants in current circulation are selected from: variant B.1.177; variant B.1.160, variant B.1.1.7; variant B.1.351; variant P.1; variant B.1.427/B.1.429; variant B.1.258; variant B.1.221; variant B.1.367; variant B.1.1.277; variant B.1.1.302; variant B.1.525; variant B.1.526, variant S: 677H; variant S: 677P; B.1.617.2-Delta, variant B.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5) and the one or more coronaviruses that cause the common cold are selected from: 229E alpha coronavirus, NL63 alpha coronavirus, OC43 beta coronavirus, and HKU1 beta coronavirus.

In some embodiments, the conserved portion of the influenza hemagglutinin (HA) protein is highly conserved among human influenza viruses. For example, the portion of the influenza hemagglutinin (HA) protein is derived from one or more of: H1N1 virus strain, H3N2 virus strain, or influenza B virus strains, wherein the H1N1 virus strains or variants are selected from: OK384178.1, OM642156.1, OM654386.1, OL840606.1, OK625377.1, OM865246.1, OM935941.1, OM642158.1, OM935953.1, MW840068.1, MW839847.1, MW839825.1, MW930730.1, MT227010.1, LC638096.1, LC638077.1, LC637999.1, and LC645067.1; wherein the H3N2 virus strains or variants are selected from: MZ005227.1, MW849238.1, MZ203409.1, MZ198318.1, MZ198312.1, MZ198295.1, MZ198289.1, MZ198265.1, MW789449.1, MW798370.1, MW790182.1, MW789645.1, MW789778.1, MW789685.1, MW789659.1, and MW790001.1 and wherein the influenza B virus strains or variants are selected from: M10298.1, MT738525.1, MT808088.1, MT056751.1, MT314641.1, MT874090.1, MT242979.1, MT315665.1, MT1055640.1, MT057563.1, MT056955.1, MT243019.1, MT306916.1, MT057571.1, MT314793.1, MT343026.1, MT874109.1, MT243795.1, MT315769.1, and KX885875.1. In certain embodiments, the conserved portion of the influenza hemagglutinin (HA) protein is encoded by one of SEQ ID NOs: 12, 14-16.

In some embodiments, the composition is encoded by one of SEQ ID NO: 21-53. In some embodiments, the compositions described herein further comprise or encode a T cell attracting chemokine, wherein the T cell attracting chemokine is CCL5, CXCL9, CXCL10, CXCL11, or a combination thereof. Alternatively, or in addition to, in some embodiments, the compositions described herein further comprising or encoding a composition that promotes T cell proliferation and T-cell memory, wherein the composition that promotes T cell proliferation and memory is IL-7, IL-2, or IL-15.

One of the unique and inventive technical features of the present invention is the use of highly conserved regions of a coronavirus spike glycoprotein(S) and a highly conserved region of an influenza hemagglutinin (HA) protein. Without wishing to limit the invention to any theory or mechanism, it is believed that the technical feature of the present invention advantageously provides for broader protection from coronaviruses and influenza viruses.

The present invention features pan-coronavirus-influenza vaccine compositions. For example, the present invention features a pre-emptive pan-Coronavirus-influenza vaccine composition comprising: at least one of SEQ ID NO: 1, 3, 5-11; and at least one of SEQ ID NO: 12, 14-16. The present invention also features a pre-emptive pan-Coronavirus-influenza vaccine composition comprising: at least one of SEQ ID NO: 1, 3, 5-11 or at least one protein encoded by SEQ ID NO: 1, 3, 5-11; and at least one of SEQ ID NO: 12, 14-16 or at least one protein encoded by SEQ ID NO: 12, 14-16. The present invention also features a pre-emptive pan-Coronavirus-influenza vaccine composition comprising at least a portion of at least one coronavirus protein selected from SEQ ID NO: 1, 3, 5-11, and at least a portion of at least one influenza hemagglutinin (HA) protein comprising a sequence according to SEQ ID NO: 12, 14-16. The present invention also features a pre-emptive pan-Coronavirus-influenza vaccine composition comprising at least a portion of one of SEQ ID NO: 1, 3, 5-11 or at least one coronavirus protein encoded by SEQ ID NO: 1, 3, 5-11, and at least a portion of one of SEQ ID NO: 12, 14-16 or at least one influenza hemagglutinin (HA) protein encoded by SEQ ID NO: 12, 14-16. Said compositions may further comprise a generic promoter, wherein at least one of SEQ ID NO: 1-11 and at least one of seq id no: 12 are operatively linked to the generic promoter. Said compositions may further comprise or encode a T cell attracting chemokine, wherein the T cell attracting chemokine is CCL5, CXCL9, CXCL10, CXCL11, or a combination thereof. Said compositions may further comprise or encode a composition that promotes T cell proliferation and T-cell memory, wherein the composition that promotes T cell proliferation and memory is IL-7, IL-2, or IL-15.

The present invention also features a pre-emptive pan-Coronavirus-influenza vaccine composition, wherein the composition comprises, or comprises a sequence encoding, at least a portion of a Coronavirus spike(S) protein and at least a portion of at least one influenza hemagglutinin (HA) protein. The portion of the coronavirus spike(S) protein is highly conserved among human and animal coronaviruses. In some embodiments, the portion of the coronavirus spike(S) protein is derived from one or more of: one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animals receptive to coronaviruses; or one or more coronaviruses that cause the common cold. The one or more SARS-CoV-2 human variants in current circulation include but are not limited to: variant B.1.177; variant B.1.160, variant B.1.1.7; variant B.1.351; variant P.1; variant B.1.427/B.1.429; variant B.1.258; variant B.1.221; variant B.1.367; variant B.1.1.277; variant B.1.1.302; variant B.1.525; variant B.1.526, variant S: 677H; variant S: 677P; B.1.617.2-Delta, variant B.1.1.529-Omicron (BA.1); sub-variant Omicron (BA.1); sub-variant Omicron (BA.2); sub-variant Omicron (BA.3); sub-variant Omicron (BA.4); and sub-variant Omicron (BA.5).

In some embodiments, the one or more coronaviruses that cause the common cold are selected from: 229E alpha coronavirus, NL63 alpha coronavirus, OC43 beta coronavirus, and HKU1 beta coronavirus. In some embodiments, the portion of the coronavirus spike(S) protein is derived from a full-length spike glycoprotein. In some embodiments, the portion of the coronavirus spike(S) protein is derived from a partial spike glycoprotein.

In some embodiments, the composition comprises at least one proline substitution. In some embodiments, the composition comprises at least two proline substitutions. In some embodiments, the composition comprises at least four proline substitutions. In some embodiments, the composition comprises at least six proline substitutions. In some embodiments, the portion of the coronavirus spike(S) protein comprises two consecutive proline substitutions at amino acid positions 986 and 987. In some embodiments, the proline substitutions are K986P and V987P mutations. In some embodiments, the portion of the coronavirus spike(S) protein is receptor-binding domain (RBD). In some embodiments, the RBD comprises a trimerized SARS-CoV-2 receptor-binding domain (RBD). In some embodiments, the portion of the coronavirus spike(S) protein comprises SEQ ID NO: 2 or 4. In some embodiments, the portion of the coronavirus spike protein is encoded by one of SEQ ID NOs: 1, 3, 5-7.

In some embodiments, the portion of the influenza hemagglutinin (HA) protein is highly conserved among human influenza viruses. In some embodiments, the portion of an influenza hemagglutinin (HA) protein is derived from one or more of: H1N1 virus strain, H3N2 virus strain, influenza B virus strains, or variants thereof. In some embodiments, the portion of the influenza hemagglutinin (HA) protein comprises SEQ ID NO: 13. In some embodiments, the portion of the influenza hemagglutinin (HA) protein is encoded by one of SEQ ID NOs: 12, 14-16.

The portion of a coronavirus spike(S) protein and the portion of the influenza hemagglutinin (HA) protein may be both/each operatively linked to a generic promoter. In some embodiments, the generic promoter is a CMV or a CAG promoter. In some embodiments, the portion of a coronavirus spike(S) protein and the portion of the influenza hemagglutinin (HA) protein are separated by a linker. The linker may be of an appropriate size, e.g., 2-10 amino acids, for example.

In some embodiments, the composition is encoded by a sequence according to SEQ ID NO: 21-53.

The present invention also features a pre-emptive pan-coronavirus-influenza vaccine composition, the composition comprising a sequence according to SEQ ID NO: 21-53. The present invention also features a pre-emptive pan-coronavirus-influenza vaccine composition comprising a sequence according to one of SEQ ID NO: 21-53 or a protein encoded by one of SEQ ID NO: 21-53. The present invention also features a pre-emptive pan-coronavirus-influenza vaccine composition comprising, or comprising a sequence encoding at least a portion of a coronavirus protein, the coronavirus protein being selected from: a structural protein, wherein the structural protein comprises a Spike protein, a Nucleocapsid protein, or a combination thereof; or a non-structural protein, wherein the non-structural protein comprises NSP2, NSP3, NSP14, or combination thereof; and at least a portion of at least one influenza hemagglutinin (HA) protein. In some embodiments, the Spike protein comprises SEQ ID NO: 2 or 4 or is encoded by one of SEQ ID NO: 1, 3, 5-7. In some embodiments, the NSP2 sequence is encoded by SEQ ID NO: 8, the NSP sequence is encoded by SEQ ID NO: 9, and the NSP14 sequence is encoded by SEQ ID NO: 10. In some embodiments, the Nucleocapsid protein is encoded by SEQ ID NO: 11. In some embodiments, the HA protein comprises SEQ ID NO: 13 or is encoded by one of SEQ ID NO: 12, 14-16.

The present invention also features a pre-emptive pan-coronavirus-influenza vaccine composition, the composition comprising: at least one of SEQ ID NO: 1, 3, 5-11; and at least one of SEQ ID NO: 12, 14-16. In some embodiments, the composition further comprises a generic promoter, wherein the at least one of SEQ ID NO: 1, 3, 5-11 and the at least one of SEQ ID NO: 12, 14-16 are operatively linked to the generic promoter.

The present invention also features a pre-emptive pan-coronavirus-influenza vaccine composition, the composition comprising, or comprising a sequence encoding one or more large coronavirus sequences or proteins and one or more large influenza sequences or proteins. In some embodiments, the one or more large coronavirus sequences comprise or encode a structural protein, a non-structural protein, or a combination therein. In some embodiments, the structural proteins are selected from: a Spike Protein or a Nucleocapsid protein, and the non-structural proteins are selected from: NSP2, NSP3, and NSP14.

The large coronavirus sequences are highly conserved among human and animal coronaviruses. In some embodiments, the one or more large coronavirus sequences are derived from one or more of: one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animals receptive to coronaviruses; one or more coronaviruses that cause the common cold; or one or more coronaviruses that case past, current, and future outbreaks. In some embodiments, the one or more SARS-CoV-2 human variants in current circulation are selected from: variant B.1.177; variant B.1.160, variant B.1.1.7; variant B.1.351; variant P.1; variant B.1.427/B.1.429; variant B.1.258; variant B.1.221; variant B.1.367; variant B.1.1.277; variant B.1.1.302; variant B.1.525; variant B.1.526, variant S: 677H; variant S: 677P; B.1.617.2-Delta, variant B.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5). In some embodiments, the one or more coronaviruses that cause the common cold are selected from: 229E alpha coronavirus, NL63 alpha coronavirus, OC43 beta coronavirus, and HKU1 beta coronavirus.

In some embodiments, the Spike(S) protein is derived from a full-length spike glycoprotein. In some embodiments, the Spike(S) protein is derived from a partial spike glycoprotein. In some embodiments, the Spike(S) protein further comprises at least one proline substitution. In some embodiments, the Spike(S) protein comprises a receptor-binding domain (RBD). In some embodiments, the RBD comprises a trimerized SARS-CoV-2 receptor-binding domain (RBD). In some embodiments, the one or more large coronavirus sequences comprises one of SEQ ID NOs: 1, 3, 5-11. In some embodiments, the one or more large influenza sequences are highly conserved among human influenza viruses. In some embodiments, the one or more large influenza sequences are derived from one or more of: H1N1 virus strain, H3N2 virus strain, or influenza B virus strains. In some embodiments, the one or more large influenza sequences comprises one of SEQ ID NOs: 12, 14-16. In some embodiments, the composition further comprises or encodes a T cell attracting chemokine, wherein the T cell attracting chemokine is CCL5, CXCL9, CXCL10, CXCL11, or a combination thereof. In some embodiments, the composition further comprises or encodes a composition that promotes T cell proliferation and T-cell memory, wherein the composition that promotes T cell proliferation and memory is IL-7, IL-2, or IL-15.

Referring to any of the embodiments herein, the H1N1 virus strains or variants may be selected from: OK384178.1, OM642156.1, OM654386.1, OL840606.1, OK625377.1, OM865246.1, OM935941.1, OM642158.1, OM935953.1, MW840068.1, MW839847.1, MW839825.1, MW930730.1, MT227010.1, LC638096.1, LC638077.1, LC637999.1, and LC645067.1.

Referring to any of the embodiments herein, the H3N2 virus strains or variants may be selected from: MZ005227.1, MW849238.1, MZ203409.1, MZ198318.1, MZ198312.1, MZ198295.1, MZ198289.1, MZ198265.1, MW789449.1, MW798370.1, MW790182.1, MW789645.1, MW789778.1, MW789685.1, MW789659.1, and MW790001.1.

Referring to any of the embodiments herein, the influenza B virus strains or variants may be selected from: M10298.1, MT738525.1, MT808088.1, MT056751.1, MT314641.1, MT874090.1, MT242979.1, MT315665.1, MT1055640.1, MT057563.1, MT056955.1, MT243019.1, MT306916.1, MT057571.1, MT314793.1, MT343026.1, MT874109.1, MT243795.1, MT315769.1, and KX885875.1.

Referring to any of the embodiments herein, the vaccine composition may further comprise a T cell attracting chemokine, wherein the T cell attracting chemokine is CCL5, CXCL9, CXCL10, CXCL11, or a combination thereof. In some embodiments, the vaccine composition further comprises a sequence encoding a composition that promotes T cell proliferation and T-cell memory, wherein the composition that promotes T cell proliferation and memory is IL-7, IL-2, or IL-15.

In some embodiments, the vaccine composition induces strong and long-lasting protection mediated by antibodies (Abs), CD4+T helper (Th1) cells, and/or CD8+ cytotoxic T-cells. In some embodiments, the composition protects against Sarbecoviruses, wherein sarbecoviruses comprise SARS-CoV1 or SARS-CoV2.

The compositions herein elicit an immune response in subjects. The compositions herein are for preventing an infection or reinfection, e.g., by one or more coronavirus variants or subvariants and influenza, in a subject.

The present invention also includes a pre-emptive pan-coronavirus-influenza vaccine composition comprising, or comprising a sequence encoding: at least a portion of a conserved coronavirus Spike protein; and at least a portion of one or more influenza HA antigens. In some embodiments, the influenza HA antigens are selected from: HA-H1N1, HA-H3N2, and HA-Influenza B. In some embodiments, the composition comprises at least a portion of a conserved coronavirus Spike protein; and at least a portion of influenza HA-H1N1 antigen. In some embodiments, the composition comprises at least a portion of a conserved coronavirus Spike protein; and at least a portion of influenza HA-H3N2 antigen. In some embodiments, the composition comprises at least a portion of a conserved coronavirus Spike protein; and at least a portion of influenza HA-Influenza B antigen. In some embodiments, the composition comprises at least a portion of a conserved coronavirus Spike protein; and two or more of HA-H1N1 antigen, HA-H3N2 antigen, and HA-Influenza B antigen. In some embodiments, the Spike protein comprises one or more proline substitutions.

The present invention also includes a pre-emptive pan-coronavirus-influenza vaccine composition comprising, or comprising a sequence encoding: at least a portion of a conserved coronavirus NSP2 protein; and at least a portion of one or more influenza HA antigens. In some embodiments, the influenza HA antigens are selected from: HA-H1N1, HA-H3N2, and HA-Influenza B. In some embodiments, the composition comprises at least a portion of a conserved coronavirus NSP2 protein; and at least a portion of influenza HA-H1N1 antigen. In some embodiments, the composition comprises at least a portion of a conserved coronavirus NSP2 protein; and at least a portion of influenza HA-H3N2 antigen. In some embodiments, the composition comprises at least a portion of a conserved coronavirus NSP2 protein; and at least a portion of influenza HA-Influenza B antigen. In some embodiments, the composition comprises at least a portion of a conserved coronavirus NSP2 protein; and two or more of HA-H1N1 antigen, HA-H3N2 antigen, and HA-Influenza B antigen.

The present invention also includes a pre-emptive pan-coronavirus-influenza vaccine composition comprising, or comprising a sequence encoding: at least a portion of a conserved coronavirus NSP3 protein; and at least a portion of one or more influenza HA antigens. In some embodiments, the influenza HA antigens are selected from: HA-H1N1, HA-H3N2, and HA-Influenza B. In some embodiments, the composition comprises at least a portion of a conserved coronavirus NSP3 protein; and at least a portion of influenza HA-H1N1 antigen. In some embodiments, the composition comprises at least a portion of a conserved coronavirus NSP3 protein; and at least a portion of influenza HA-H3N2 antigen. In some embodiments, the composition comprises at least a portion of a conserved coronavirus NSP3 protein; and at least a portion of influenza HA-Influenza B antigen. In some embodiments, the composition comprises at least a portion of a conserved coronavirus NSP3 protein; and two or more of HA-H1N1 antigen, HA-H3N2 antigen, and HA-Influenza B antigen.

The present invention also includes a pre-emptive pan-coronavirus-influenza vaccine composition comprising, or comprising a sequence encoding: at least a portion of a conserved coronavirus NSP14 protein; and at least a portion of one or more influenza HA antigens. In some embodiments, the influenza HA antigens are selected from: HA-H1N1, HA-H3N2, and HA-Influenza B. In some embodiments, the composition comprises at least a portion of a conserved coronavirus NSP41 protein; and at least a portion of influenza HA-H1N1 antigen. In some embodiments, the composition comprises at least a portion of a conserved coronavirus NSP41 protein; and at least a portion of influenza HA-H3N2 antigen. In some embodiments, the composition comprises at least a portion of a conserved coronavirus NSP14 protein; and at least a portion of influenza HA-Influenza B antigen. In some embodiments, the composition comprises at least a portion of a conserved coronavirus NSP14 protein; and two or more of HA-H1N1 antigen, HA-H3N2 antigen, and HA-Influenza B antigen.

The present invention also includes a pre-emptive pan-coronavirus-influenza vaccine composition comprising, or comprising a sequence encoding: at least a portion of a conserved coronavirus Nucleoprotein (nucleocapsid); and at least a portion of one or more influenza HA antigens. In some embodiments, the influenza HA antigens are selected from: HA-H1N1, HA-H3N2, and HA-Influenza B. In some embodiments, the composition comprises at least a portion of a conserved coronavirus Nucleoprotein (nucleocapsid); and at least a portion of influenza HA-H1N1 antigen. In some embodiments, the composition comprises at least a portion of a conserved coronavirus Nucleoprotein (nucleocapsid); and at least a portion of influenza HA-H3N2 antigen. In some embodiments, the composition comprises at least a portion of a conserved coronavirus Nucleoprotein (nucleocapsid); and at least a portion of influenza HA-Influenza B antigen. In some embodiments, the composition comprises at least a portion of a conserved coronavirus Nucleoprotein (nucleocapsid); and two or more of HA-H1N1 antigen, HA-H3N2 antigen, and HA-Influenza B antigen.

The present invention also includes a pre-emptive pan-coronavirus-influenza vaccine composition comprising, or comprising a sequence encoding: at least two conserved coronavirus antigens selected from: (i) a conserved coronavirus Spike protein; (ii) a conserved coronavirus NSP2 protein; (iii) a conserved coronavirus NSP3 protein; (iv) a conserved coronavirus NSP14 protein; and (v) a conserved coronavirus Nucleoprotein (nucleocapsid); and at least a portion of one or more influenza HA antigens. The aforementioned proteins or antigens may refer to portions of a particular entire protein. Thus, the present invention also includes a pre-emptive pan-coronavirus-influenza vaccine composition comprising, or comprising a sequence encoding: at least two conserved coronavirus antigens selected from: (i) a conserved coronavirus Spike protein or a portion thereof; (ii) a conserved coronavirus NSP2 protein or a portion thereof; (iii) a conserved coronavirus NSP3 protein or a portion thereof; (iv) a conserved coronavirus NSP14 protein or a portion thereof; and (v) a conserved coronavirus Nucleoprotein (nucleocapsid) or a portion thereof; and at least a portion of one or more influenza HA antigens. In some embodiments, the influenza HA antigens are selected from: HA-H1N1, HA-H3N2, and HA-Influenza B.

In some embodiments, the composition comprises a conserved coronavirus Spike protein (or a portion thereof); and a conserved coronavirus NSP2 protein (or a portion thereof); and at least a portion of one or more influenza HA antigens. In some embodiments, the composition comprises a conserved coronavirus Spike protein (or a portion thereof); and a conserved coronavirus NSP3 protein (or a portion thereof); and at least a portion of one or more influenza HA antigens. In some embodiments, the composition comprises a conserved coronavirus Spike protein (or a portion thereof); and a conserved coronavirus NSP14 protein (or a portion thereof); and at least a portion of one or more influenza HA antigens. In some embodiments, the composition comprises a conserved coronavirus Spike protein (or a portion thereof); and a conserved coronavirus Nucleoprotein (nucleocapsid) (or a portion thereof); and at least a portion of one or more influenza HA antigens. In some embodiments, the composition comprises a conserved coronavirus Spike protein (or a portion thereof); a conserved coronavirus NSP2 protein (or a portion thereof); and a conserved coronavirus NSP3 protein (or a portion thereof); and at least a portion of one or more influenza HA antigens. In some embodiments, the composition comprises a conserved coronavirus Spike protein (or a portion thereof); a conserved coronavirus NSP2 protein (or a portion thereof); and a conserved coronavirus NSP14 protein (or a portion thereof); and at least a portion of one or more influenza HA antigens. In some embodiments, the composition comprises a conserved coronavirus Spike protein (or a portion thereof); a conserved coronavirus NSP2 protein (or a portion thereof); and a conserved coronavirus Nucleoprotein (nucleocapsid) (or a portion thereof); and at least a portion of one or more influenza HA antigens. In some embodiments, the composition comprises a conserved coronavirus Spike protein (or a portion thereof); a conserved coronavirus NSP3 protein (or a portion thereof); and a conserved coronavirus NSP14 protein (or a portion thereof); and at least a portion of one or more influenza HA antigens. In some embodiments, the composition comprises a conserved coronavirus Spike protein (or a portion thereof); a conserved coronavirus NSP3 protein (or a portion thereof); and a conserved coronavirus Nucleoprotein (nucleocapsid) (or a portion thereof); and at least a portion of one or more influenza HA antigens. In some embodiments, the composition comprises a conserved coronavirus Spike protein (or a portion thereof); a conserved coronavirus NSP14 protein (or a portion thereof); and a conserved coronavirus Nucleoprotein (nucleocapsid) (or a portion thereof); and at least a portion of one or more influenza HA antigens. In some embodiments, the Spike protein (or a portion thereof) comprises one or more proline substitutions. In some embodiments, the composition comprises a conserved coronavirus NSP2 protein (or a portion thereof); and a conserved coronavirus NSP3 protein (or a portion thereof); and at least a portion of one or more influenza HA antigens. In some embodiments, the composition comprises a conserved coronavirus NSP2 protein (or a portion thereof); and a conserved coronavirus NSP14 protein (or a portion thereof); and at least a portion of one or more influenza HA antigens. In some embodiments, the composition comprises a conserved coronavirus NSP2 protein (or a portion thereof); and a conserved coronavirus Nucleoprotein (nucleocapsid) (or a portion thereof); and at least a portion of one or more influenza HA antigens. In some embodiments, the composition comprises a conserved coronavirus NSP3 protein (or a portion thereof); and a conserved coronavirus NSP14 protein (or a portion thereof); and at least a portion of one or more influenza HA antigens. In some embodiments, the composition comprises a conserved coronavirus NSP3 protein (or a portion thereof); and a conserved coronavirus Nucleoprotein (nucleocapsid) (or a portion thereof); and at least a portion of one or more influenza HA antigens. In some embodiments, the composition comprises a conserved coronavirus NSP14 protein (or a portion thereof); and a conserved coronavirus Nucleoprotein (nucleocapsid) (or a portion thereof); and at least a portion of one or more influenza HA antigens. In some embodiments, the composition comprises a conserved coronavirus NSP2 protein (or a portion thereof); a conserved coronavirus NSP3 protein (or a portion thereof); and a conserved coronavirus NSP14 protein (or a portion thereof); and at least a portion of one or more influenza HA antigens. In some embodiments, the composition comprises a conserved coronavirus NSP2 protein (or a portion thereof); a conserved coronavirus NSP3 protein (or a portion thereof); and a conserved coronavirus Nucleoprotein (nucleocapsid) (or a portion thereof); and at least a portion of one or more influenza HA antigens. In some embodiments, the composition comprises a conserved coronavirus NSP2 protein (or a portion thereof); a conserved coronavirus NSP14 protein (or a portion thereof); and a conserved coronavirus Nucleoprotein (nucleocapsid) (or a portion thereof); and at least a portion of one or more influenza HA antigens. In some embodiments, the composition comprises a conserved coronavirus NSP3 protein (or a portion thereof); a conserved coronavirus NSP14 protein (or a portion thereof); and a conserved coronavirus Nucleoprotein (nucleocapsid) (or a portion thereof); and at least a portion of one or more influenza HA antigens. In some embodiments, the composition comprises a conserved coronavirus Spike protein (or a portion thereof); a conserved coronavirus NSP2 protein (or a portion thereof); a conserved coronavirus NSP3 protein (or a portion thereof); and a conserved coronavirus NSP14 protein (or a portion thereof); and at least a portion of one or more influenza HA antigens. In some embodiments, the composition comprises a conserved coronavirus Spike protein (or a portion thereof); a conserved coronavirus NSP2 protein (or a portion thereof); a conserved coronavirus NSP3 protein (or a portion thereof); and a conserved coronavirus Nucleoprotein (nucleocapsid) (or a portion thereof); and at least a portion of one or more influenza HA antigens. In some embodiments, the composition comprises a conserved coronavirus Spike protein (or a portion thereof); a conserved coronavirus NSP2 protein (or a portion thereof); a conserved coronavirus NSP3 protein (or a portion thereof); and a conserved coronavirus NSP14 protein (or a portion thereof); a conserved coronavirus Nucleoprotein (nucleocapsid) (or a portion thereof); and at least a portion of one or more influenza HA antigens. In some embodiments, the Spike protein (or a portion thereof) comprises one or more proline substitutions. In some embodiments, the composition comprises a conserved coronavirus NSP2 protein (or a portion thereof); a conserved coronavirus NSP3 protein (or a portion thereof); and a conserved coronavirus NSP14 protein (or a portion thereof); a conserved coronavirus Nucleoprotein (nucleocapsid) (or a portion thereof); and at least a portion of one or more influenza HA antigens.

In some embodiments, the composition further comprises a T cell attracting chemokine, wherein the T cell attracting chemokine is CCL5, CXCL9, CXCL10, CXCL11, or a combination thereof. In some embodiments, the composition further comprises a composition that promotes T cell proliferation and T-cell memory, wherein the composition that promotes T cell proliferation and memory is IL-7, IL-2, or IL-15. In some embodiments, the conserved protein or antigen is conserved among human and animal coronaviruses. In some embodiments, the portion of the coronavirus spike(S) protein is derived from a full-length spike glycoprotein. In some embodiments, the portion of the coronavirus spike(S) protein is derived from a partial spike glycoprotein. In some embodiments, the portion of the coronavirus spike(S) protein is receptor-binding domain (RBD). In some embodiments, the RBD comprises a trimerized SARS-CoV-2 receptor-binding domain (RBD). In some embodiments, the coronavirus protein is encoded by one of SEQ ID NOs: 1, 3, 5-11. In some embodiments, the HA antigen is highly conserved among human influenza viruses. In some embodiments, the influenza HA antigen is encoded by one of SEQ ID NOs: 12, 14-16.

The present invention also includes a pre-emptive pan-coronavirus-influenza vaccine composition, the composition comprising, or comprising a sequence encoding one or more large sequence coronavirus proteins and one or more influenza antigens, wherein the one or more large sequence coronavirus proteins comprises one or more of: a conserved coronavirus Spike protein (or a portion thereof); a conserved coronavirus NSP2 protein (or a portion thereof); a conserved coronavirus NSP3 protein (or a portion thereof); a conserved coronavirus NSP14 protein (or a portion thereof); or a conserved coronavirus Nucleoprotein (nucleocapsid) (or a portion thereof).

In some embodiments, the one or more large sequence coronavirus proteins comprises two or more of: a conserved coronavirus Spike protein (or a portion thereof); a conserved coronavirus NSP2 protein (or a portion thereof); a conserved coronavirus NSP3 protein (or a portion thereof); a conserved coronavirus NSP14 protein (or a portion thereof); or a conserved coronavirus Nucleoprotein (nucleocapsid) (or a portion thereof). In some embodiments, the one or more large sequence coronavirus proteins comprises three or more of: a conserved coronavirus Spike protein (or a portion thereof); a conserved coronavirus NSP2 protein (or a portion thereof); a conserved coronavirus NSP3 protein (or a portion thereof); a conserved coronavirus NSP14 protein (or a portion thereof); or a conserved coronavirus Nucleoprotein (nucleocapsid) (or a portion thereof). In some embodiments, the one or more large sequence coronavirus proteins comprises four or more of: a conserved coronavirus Spike protein (or a portion thereof); a conserved coronavirus NSP2 protein (or a portion thereof); a conserved coronavirus NSP3 protein (or a portion thereof); a conserved coronavirus NSP14 protein (or a portion thereof); or a conserved coronavirus Nucleoprotein (nucleocapsid) (or a portion thereof). In some embodiments, the one or more large sequence coronavirus proteins comprises: a conserved coronavirus Spike protein (or a portion thereof); a conserved coronavirus NSP2 protein (or a portion thereof); a conserved coronavirus NSP3 protein (or a portion thereof); a conserved coronavirus NSP14 protein (or a portion thereof); and a conserved coronavirus Nucleoprotein (nucleocapsid) (or a portion thereof). In some embodiments, the composition comprises one or more influenza antigens selected from: HA-H1N1 antigen, HA-H3N2 antigen, and HA-Influenza B antigen, or portions thereof. In some embodiments, the composition comprises two or more influenza antigens selected from: HA-H1N1 antigen, HA-H3N2 antigen, and HA-Influenza B antigen. In some embodiments, the composition comprises HA-H1N1 antigen, HA-H3N2 antigen, and HA-Influenza B antigen. In some embodiments, the large coronavirus sequences are highly conserved among human and animal coronaviruses. In some embodiments, the Spike(S) protein further comprises at least one proline substitution. In some embodiments, the Spike(S) protein comprises a receptor-binding domain (RBD). In some embodiments, the RBD comprises a trimerized SARS-CoV-2 receptor-binding domain (RBD). In some embodiments, the one or more large coronavirus sequences comprises one of SEQ ID NOs: 1, 3, 5-11. In some embodiments, the one or more large influenza sequences are highly conserved among human influenza viruses. In some embodiments, the one or more large influenza sequences comprises one of SEQ ID NOs: 12, 14-16. In some embodiments, the composition further comprises a T cell attracting chemokine, wherein the T cell attracting chemokine is CCL5, CXCL9, CXCL10, CXCL11, or a combination thereof. In some embodiments, the composition further comprises a composition that promotes T cell proliferation and T-cell memory, wherein the composition that promotes T cell proliferation and memory is IL-7, IL-2, or IL-15.

The present invention also features a method of preventing infection or reinfection by one or more coronavirus variants or subvariants and influenza in a subject, said method comprising administering a therapeutically effective amount of a composition according to the present invention.

The specification of U.S. Application No. 63/349,799 filed Jun. 7, 2022 is hereby incorporated herein in its entirety by reference.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows the results of a sequence alignment of various influenza viruses and variants and the resulting conserved region.

FIG. 2 shows non-limiting examples of vaccine compositions described herein. For example, in some embodiments, the vaccine composition comprises a spike protein domain (optionally with a mutation such as stabilizing mutations and/or proline substitutions), e.g., a coronavirus spike protein or fragment thereof or derivative thereof, and an influenza HA domain or portion thereof or derivative thereof. In some embodiments, the vaccine compositions further comprise a promoter, e.g., CAG, CMV, etc. In some embodiments, the vaccine compositions comprise a signal peptide. In some embodiments, the spike protein domain and HA domain are separated by a linker. In some embodiments, the vaccine composition comprises a spike protein domain (optionally with a mutation such as stabilizing mutations and/or proline substitutions), e.g., a coronavirus spike protein or fragment thereof or derivative thereof, and two or more influenza HA domains or portion thereof or derivative thereof, e.g., HA-H1N1 and/or HA-H3N2 and/or HA-Influenza B. In some embodiments, the two or more HA domains are selected from HA-H1N1, HA-H3N2, HA-Influenza B, and/or an HA conserved region. In some embodiments, the two or more HA domains are separated by a linker.

The terms S1 and S2 merely refer to subunits of the spike protein.

FIG. 3A shows non-limiting examples of influenza proteins and SARS-CoV2 proteins that may be used in the vaccine compositions as described herein; these proteins may be used in any combination to create said compositions. In FIG. 3A, “Nsp” may refer to Nsp1, Nsp2, Nsp3, Nsp5, Nsp6, Nsp7, Nsp8, Nsp9, Nsp10, Nsp12, Nsp13, Nsp14, Nsp15, Nsp16, or a combination thereof.

FIG. 3B shows non-limiting examples of hybrid vaccine compositions described herein. The proteins may be covalently or non-covalently linked together for the administration of the vaccine composition. In FIG. 3B, “Nsp” may refer to Nsp1, Nsp2, Nsp3, Nsp5, Nsp6, Nsp7, Nsp8, Nsp9, Nsp10, Nsp12, Nsp13, Nsp14, Nsp15, Nsp16, a portion thereof, or a combination thereof, “Spike protein” may refer to a portion of the spike protein, or a spike protein with one or more mutations (e.g., a spike protein with two or six proline substitutions); the spike protein or portion thereof is not limited to those comprising one or more mutations.

FIG. 3C shows non-limiting examples of hybrid vaccine compositions described herein. The proteins may be covalently or non-covalently linked together for the administration of the vaccine composition. In FIG. 3C, “HA” may refer to an influenza hemagglutinin (HA) protein may be derived from one or more of: the H1N1 virus strain, the H3N2 virus strain, influenza B virus strains, or variants thereof, “Spike protein” may refer to a portion of the spike protein, or a spike protein with one or more mutations (e.g., a spike protein with two or six proline substitutions); the spike protein or portion thereof is not limited to those comprising one or more mutations.

### TERMS

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessarily solely.” Furthermore, variations of the word “comprising,” such as “comprise” and “comprises,” have correspondingly the same meanings. In one respect, the technology described herein is related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

Although methods and materials similar or equivalent to those described herein can be used to practice or test the disclosed technology, suitable methods and materials are described below. The materials, methods, and examples are illustrative only and are not intended to be limiting.

As used herein, the term “immunological response” to a composition or vaccine refers to the development in the host of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. The host may display either a therapeutic or protective immunological response, so resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

As used herein, the terms “immunogenic protein, polypeptide, or peptide” or “antigen” refer to polypeptides or other molecules (or combinations of polypeptides and other molecules) that are immunologically active in the sense that once administered to the host, it is able to evoke an immune response of the humoral and/or cellular type directed against the protein. In embodiments, the protein fragment has substantially the same immunological activity as the total protein. Thus, a protein fragment according to the disclosure can comprises or consists essentially of or consists of at least one epitope or antigenic determinant. An “immunogenic” protein or polypeptide, as used herein, may include the full-length sequence of the protein, analogs thereof, or immunogenic fragments thereof. “Immunogenic fragment” refers to a fragment of a protein that includes one or more epitopes and thus elicits the immunological response described above.

Synthetic antigens are also included within the definition, for example, poly-epitopes, flanking epitopes, and other recombinant or synthetically derived antigens. Immunogenic fragments for purposes of the disclosure may feature at least about 1 amino acid, at least about 3 amino acids, at least about 5 amino acids, at least about 10-15 amino acids, or about 15-25 amino acids or more amino acids, of the molecule. There is no critical upper limit to the length of the fragment, which could comprise nearly the full-length of the protein sequence, or the full-length of the protein sequence, or even a fusion protein comprising at least one epitope of the protein.

As used herein, the term “variant” refers to a substantially similar sequence. For polynucleotides, a variant comprises a deletion and/or addition and/or change of one or more nucleotides at one or more sites within the native polynucleotide and/or a substitution of one or more nucleotides at one or more sites in the native polynucleotide. As used herein, a “native” polynucleotide or polypeptide comprises a naturally occurring nucleotide sequence or an amino acid sequence, respectively. Variants of a particular polynucleotide of the disclosure (e.g., the reference polynucleotide) can also be evaluated by comparison of the percent sequence identity between the polypeptide encoded by a variant polynucleotide and the polypeptide encoded by the reference polynucleotide. “Variant” protein is intended to mean a protein derived from the native protein by deletion or addition of one or more amino acids at one or more sites in the native protein and/or substitution of one or more amino acids at one or more sites in the native protein. Variant proteins encompassed by the present disclosure are biologically active; that is they have the ability to elicit an immune response.

As used herein, the terms “treat” or “treatment” or “treating” refers to both therapeutic treatment and prophylactic or preventative measures, wherein the object is to prevent or slow the development of the disease, such as slow down the development of a disorder, or reducing at least one adverse effect or symptom of a condition, disease or disorder, e.g., any disorder characterized by insufficient or undesired organ or tissue function. Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” also includes ameliorating a disease, lessening the severity of its complications, preventing it from manifesting, preventing it from recurring, merely preventing it from worsening, mitigating an inflammatory response included therein, or a therapeutic effort to affect any of the aforementioned, even if such therapeutic effort is ultimately unsuccessful.

As used herein, the term “carrier,” or “pharmaceutically acceptable carrier,” or “pharmaceutically acceptable vehicle” refers to any appropriate or useful carrier or vehicle for introducing a composition to a subject. Pharmaceutically acceptable carriers or vehicles may be conventional but are not limited to conventional vehicles. For example, E. W. Martin, Remington's Pharmaceutical Sciences, Mack Publishing Co., Easton, PA, 15th Edition (1975) and D. B. Troy, ed. Remington: The Science and Practice of Pharmacy, Lippincott Williams & Wilkins, Baltimore MD and Philadelphia, PA, 21st Edition (2006) describe compositions and formulations suitable for pharmaceutical delivery of one or more therapeutic compounds or molecules. Carriers (e.g., pharmaceutical carriers, pharmaceutical vehicles, pharmaceutical compositions, pharmaceutical molecules, etc.) are materials generally known to deliver molecules, proteins, cells and/or drugs and/or other appropriate material into the body. In general, the nature of the carrier will depend on the nature of the composition being delivered as well as the particular mode of administration being employed. In addition to biologically-neutral carriers, pharmaceutical compositions administered may contain minor amounts of non-toxic auxiliary substances, such as wetting or emulsifying agents, preservatives, and pH buffering agents and the like. Patents that describe pharmaceutical carriers include, but are not limited to: U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, the disclosures of which are incorporated in their entirety by reference herein. The carrier may, for example, be solid, liquid (e.g., a solution), foam, a gel, the like, or a combination thereof. In some embodiments, the carrier comprises a biological matrix (e.g., biological fibers, etc.). In some embodiments, the carrier comprises a synthetic matrix (e.g., synthetic fibers, etc.). In certain embodiments, a portion of the carrier may comprise a biological matrix and a portion may comprise synthetic matrix.

As used herein, “coronavirus” may refer to a group of related viruses such as but not limited to severe acute respiratory syndrome (SARS), Middle East respiratory syndrome (MERS), and severe acute respiratory syndrome coronavirus 2 (SARS-CoV-2). All the coronaviruses cause respiratory tract infections that range from mild to lethal in mammals. Several non-limiting examples of Coronavirus strains are described herein. In some embodiments, the compositions may protect against any Sarbecoviruses, including but not limited to SARS-CoV1 or SARS-CoV2. As used herein, “severe acute respiratory syndrome coronavirus 2 (SARS-CoV2)” is a betacoronavirus that causes Coronavirus Disease 19 (COVID-19).

As used herein, the term “conserved” refers to a large sequence that is among the most highly conserved large sequences identified in a sequence alignment and analysis. For example, the conserved large sequences may be the 2 most highly conserved sequences identified. In some embodiments, the conserved large sequences may be the 3 most highly conserved sequences identified. In some embodiments, the conserved large sequences may be the 4 most highly conserved sequences identified. In some embodiments, the conserved large sequences may be the 5 most highly conserved sequences identified. In some embodiments, the conserved large sequences may be the 6 most highly conserved sequences identified. In some embodiments, the conserved large sequences may be the 7 most highly conserved sequences identified. In some embodiments, the conserved large sequences may be the 8 most highly conserved sequences identified. In some embodiments, the conserved large sequences may be the 9 most highly conserved sequences identified. In some embodiments, the conserved large sequences may be the 10 most highly conserved sequences identified. In some embodiments, the conserved large sequences may be the 15 most highly conserved sequences identified. In some embodiments, the conserved large sequences may be the 20 most highly conserved sequences identified. In some embodiments, the conserved large sequences may be the 25 most highly conserved sequences identified. In some embodiments, the conserved large sequences may be the 30 most highly conserved sequences identified. In some embodiments, the conserved large sequences may be the 40 most highly conserved sequences identified. In some embodiments, the conserved large sequences may be the 50 most highly conserved sequences identified. In some embodiments, the conserved sequences may be the 50% most highly conserved large sequences identified. In some embodiments, the conserved large sequences may be the 60% most highly conserved sequences identified. In some embodiments, the large conserved sequences may be the 70% most highly conserved sequences identified. In some embodiments, the conserved large sequences may be the 80% most highly conserved sequences identified. In some embodiments, the conserved large sequences may be the 90% most highly conserved sequences identified. In some embodiments, the conserved large sequences may be the 95% most highly conserved sequences identified. In some embodiments, the conserved large sequences may be the 99% most highly conserved sequences identified. The present invention is not limited to the aforementioned thresholds.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile, or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects.

The terms “administering” and “administration” refer to methods of providing pharmaceutical preparation to a subject. Such methods are well known to those skilled in the art and include, but are not limited to, administering the compositions orally, parenterally (e.g., intravenously and subcutaneously), by intramuscular injection, by intraperitoneal injection, intrathecally, transdermally, extracorporeally, topically or the like.

A composition can be administered by topical intranasal administration (intranasally) or administration by inhalant. As used herein, “topical intranasal administration” means delivery of the compositions into the nose and nasal passages through one or both of the nares and can comprise delivery by a spraying mechanism (device) or droplet mechanism (device), or through aerosolization of the composition. Administration of the compositions by inhalant can be through the nose or mouth via delivery by a spraying or droplet mechanism. As used herein, “an inhaler” can be a spraying device or a droplet device for delivering a composition comprising the vaccine composition, in a pharmaceutically acceptable carrier, to the nasal passages and the upper and/or lower respiratory tracts of a subject. Delivery can also be directly to any area of the respiratory system (e.g., lungs) via intratracheal intubation. The exact amount of the compositions required will vary from subject to subject, depending on the species, age, weight and general condition of the subject, the severity of the disorder being treated, the particular composition used, its mode of administration and the like. Thus, it is not possible to specify an exact amount for every composition. However, an appropriate amount can be determined by one of ordinary skill in the art using only routine experimentation given the teachings herein.

A composition can also be administered by buccal delivery or by sublingual delivery. As used herein “buccal delivery” may refer to a method of administration in which the compound is delivered through the mucosal membranes lining the cheeks. In some embodiment, for a buccal delivery the vaccine composition is placed between the gum and the cheek of a patient. As used herein “sublingual delivery” may refer to a method of administration in which the compound is delivered through the mucosal membrane under the tongue. In some embodiments, for a sublingual delivery the vaccine composition is administered under the tongue of a patient.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves the use of a slow-release or sustained-release system such that a constant dosage is maintained.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting. Embodiments of the present invention can be freely combined with each other if they are not mutually exclusive.

The present invention features hybrid vaccine compositions, methods of use, methods of producing said vaccines, methods of preventing coronavirus and/or influenza infections, etc. The present invention also provides methods of testing said vaccines, e.g., using particular animal models and clinical trials. The vaccine compositions herein can induce efficient and powerful protection against the coronavirus and/or influenza disease or infection, e.g., by inducing the production of antibodies (Abs), CD4+T helper (Th1) cells, and CD8+ cytotoxic T-cells (CTL).

The vaccine compositions, e.g., the antigens herein, feature multiple large sequences, which may comprise multiple conserved epitopes, that help provide multiple opportunities for the body to develop an immune response for preventing infection. Further, the vaccines herein may be designed to be effective against past, current, and future coronavirus and/or influenza outbreaks.

The vaccine composition comprises multiple large sequences. In certain embodiments, the large sequences are conserved large sequences, e.g., sequences that are highly conserved among human coronaviruses and/or animal coronaviruses (e.g., coronaviruses isolated from animals susceptible to coronavirus infections).

The large sequences may comprise a T-cell epitope restricted to a large number of human class 1 and class 2 HLA haplotypes and not restricted to HLA-0201 for class 1 or HLA-DR for class 2. The conserved large sequences may be restricted to human HLA class 1 and 2 haplotypes. In some embodiments, the conserved epitopes are restricted to cat and dog MHC class 1 and 2 haplotypes.

The antigen may comprise large sequences, such as conserved large sequences that are highly conserved among human and animal coronaviruses. As used herein, the term large sequence refers to a sequence having at least 25 amino acids or at least 75 nucleotides. The large sequences comprise epitopes, such as the conserved epitopes described herein.

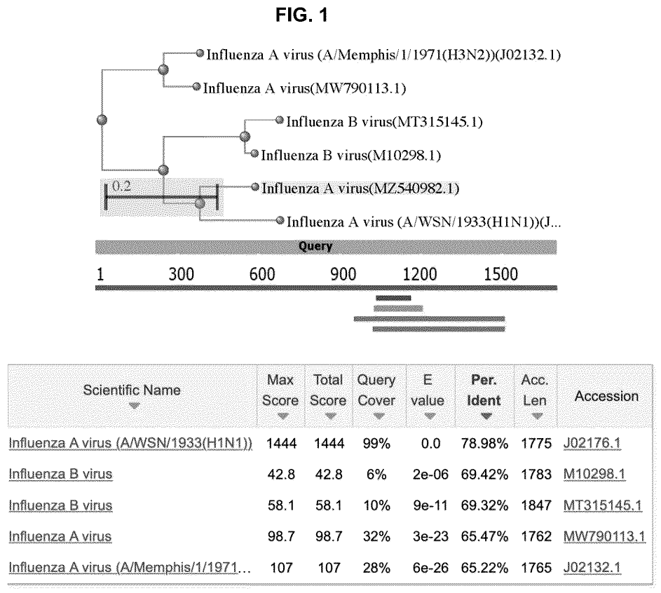

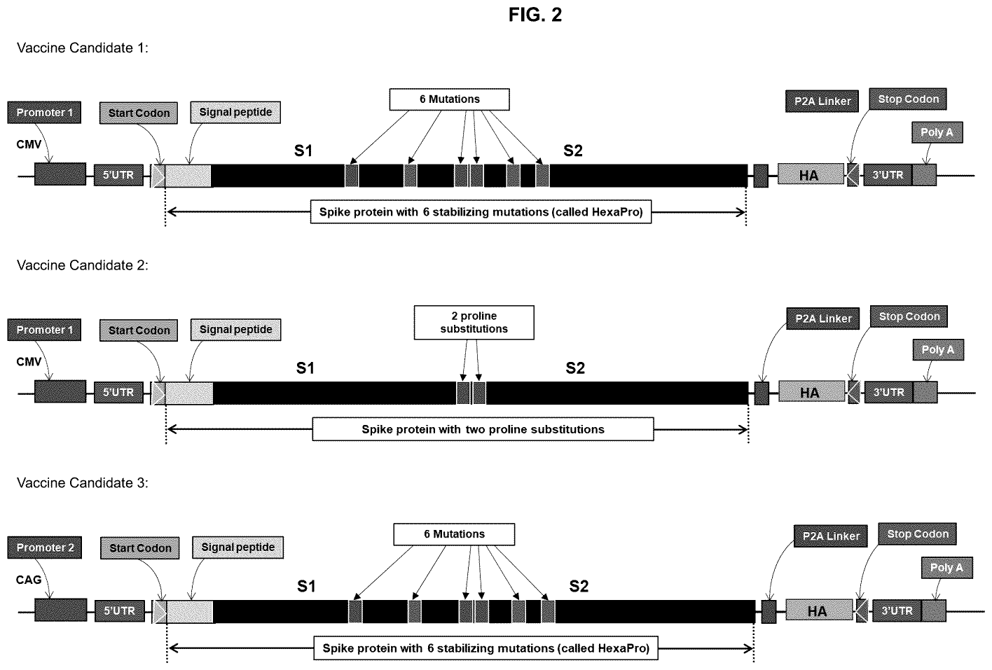

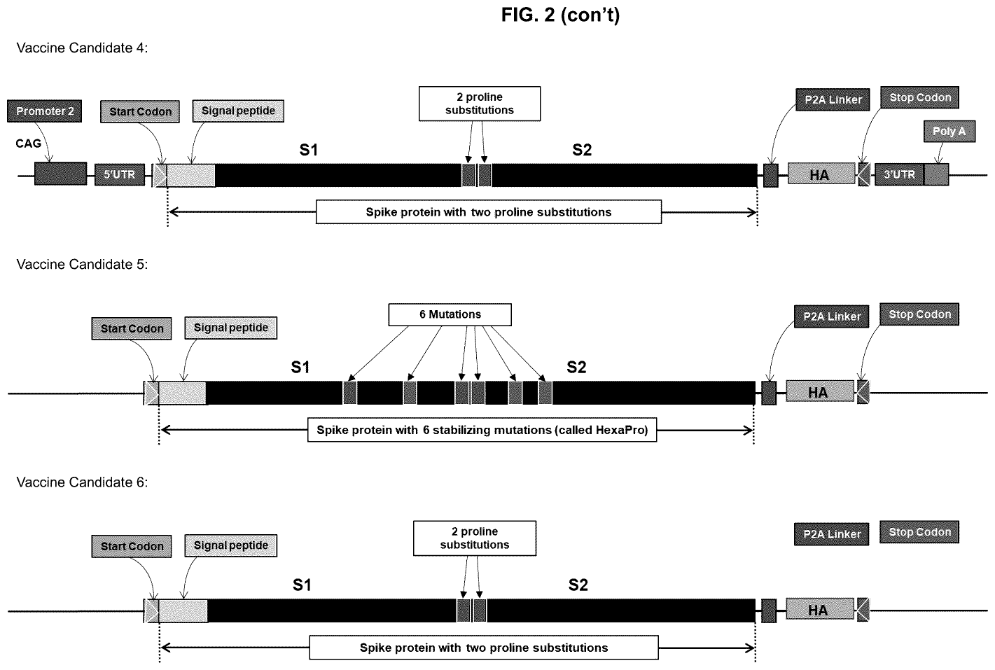

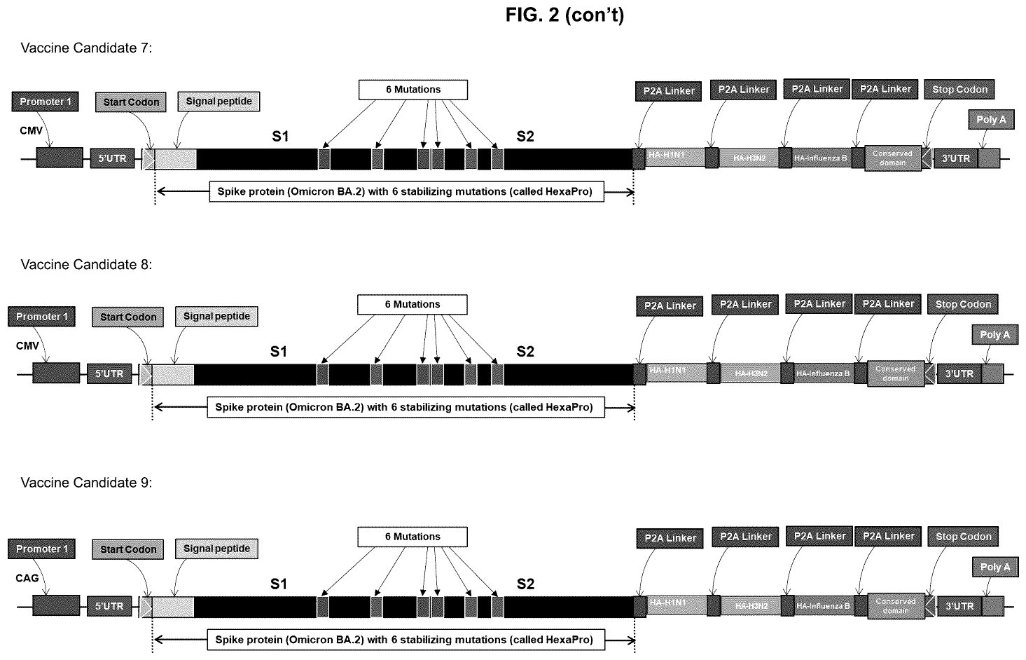

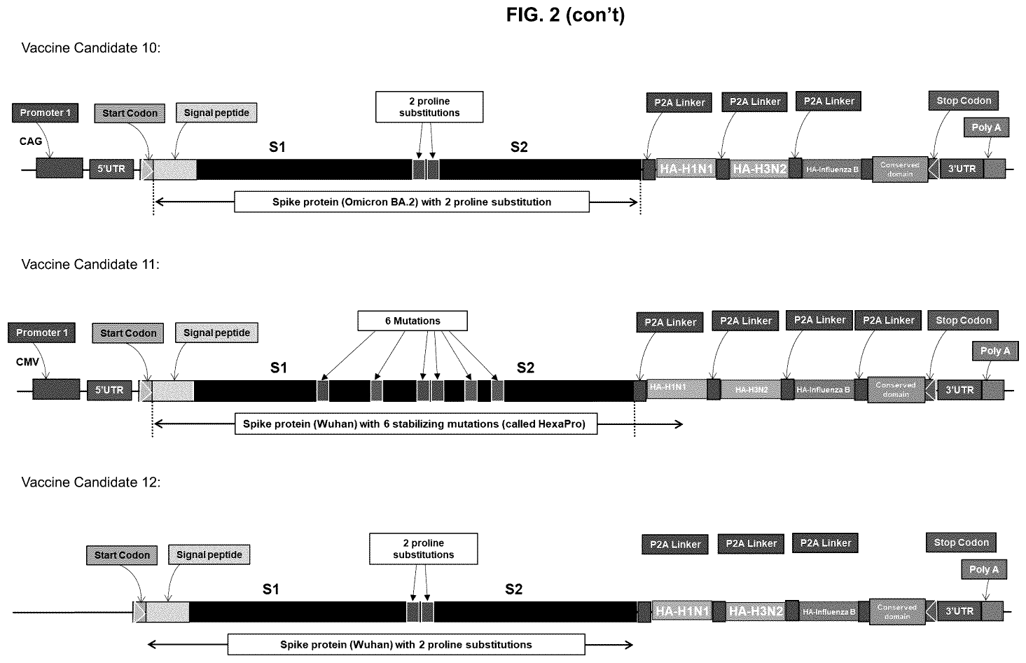

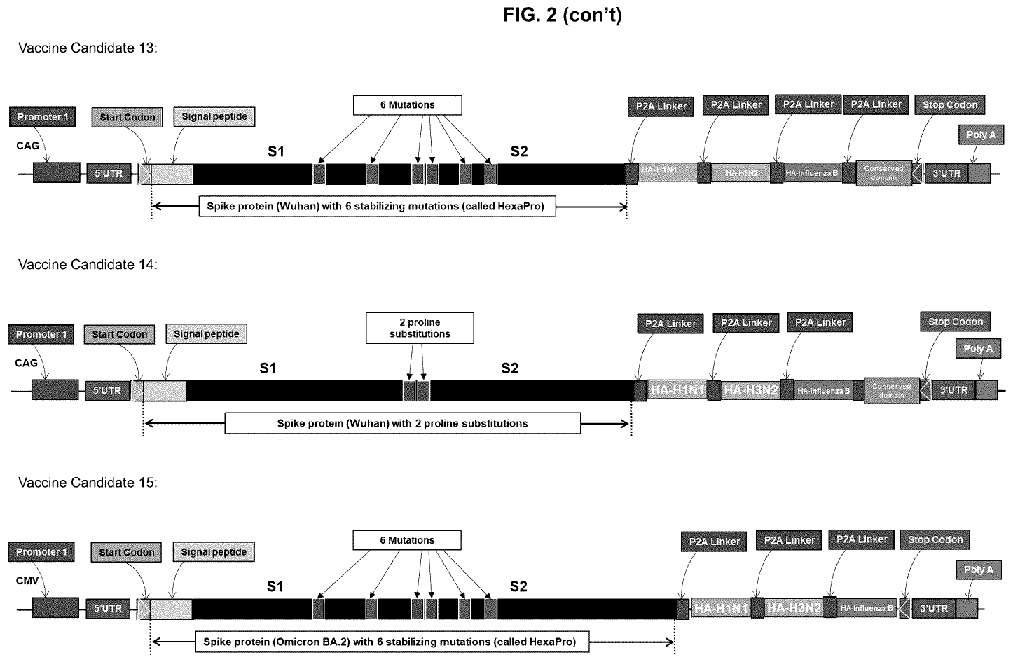

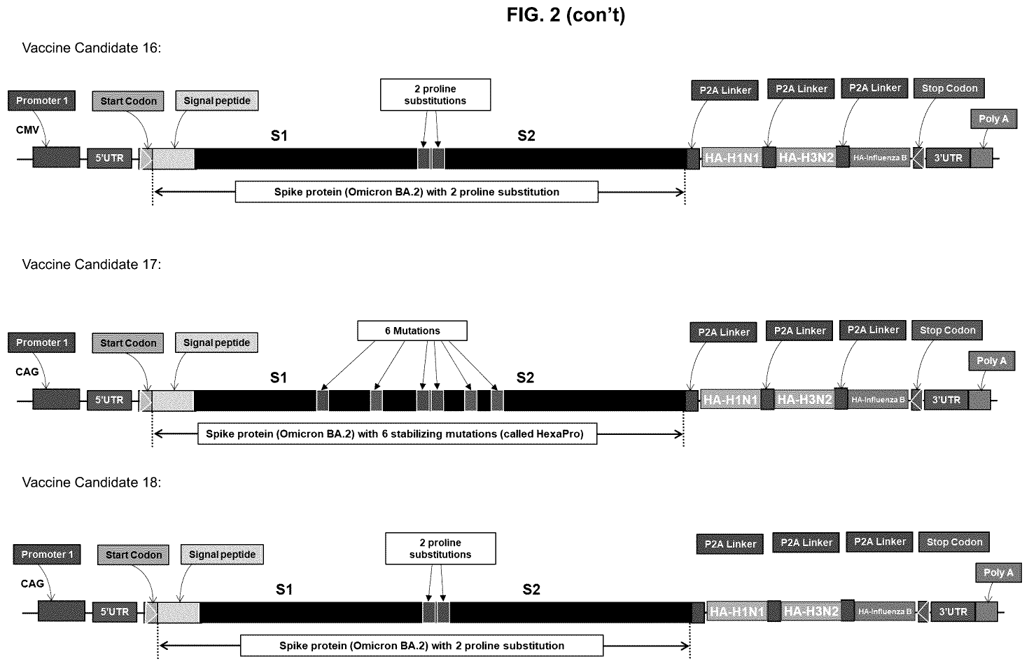

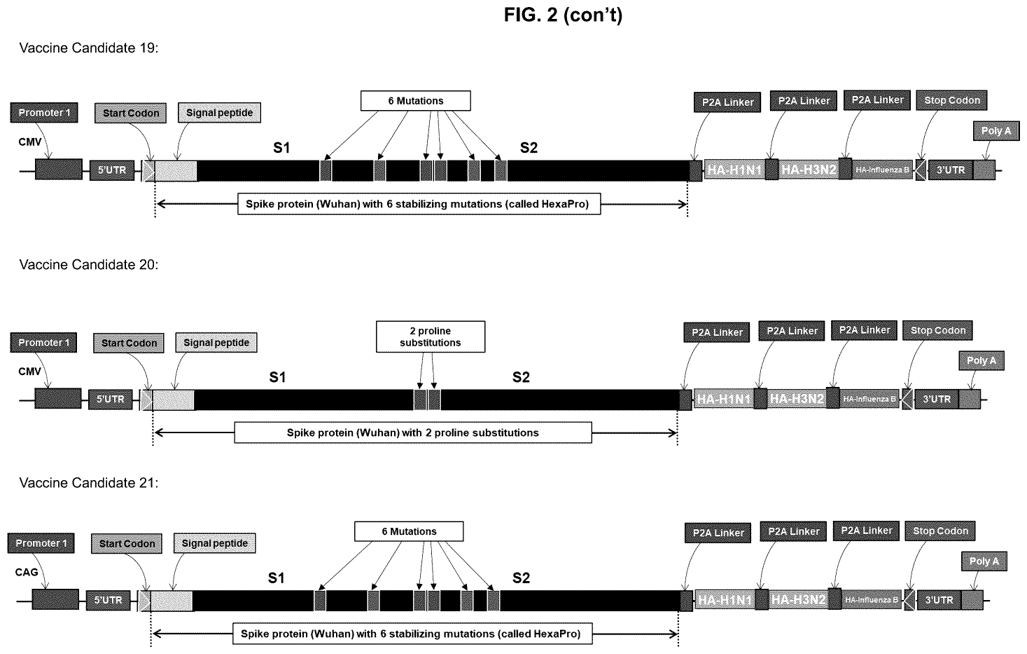

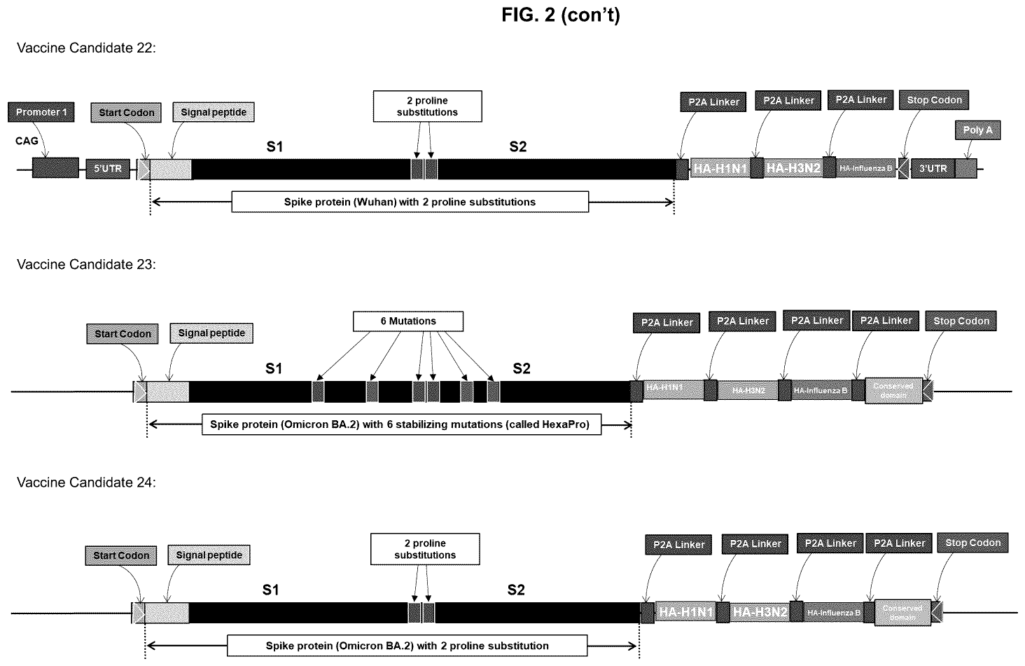

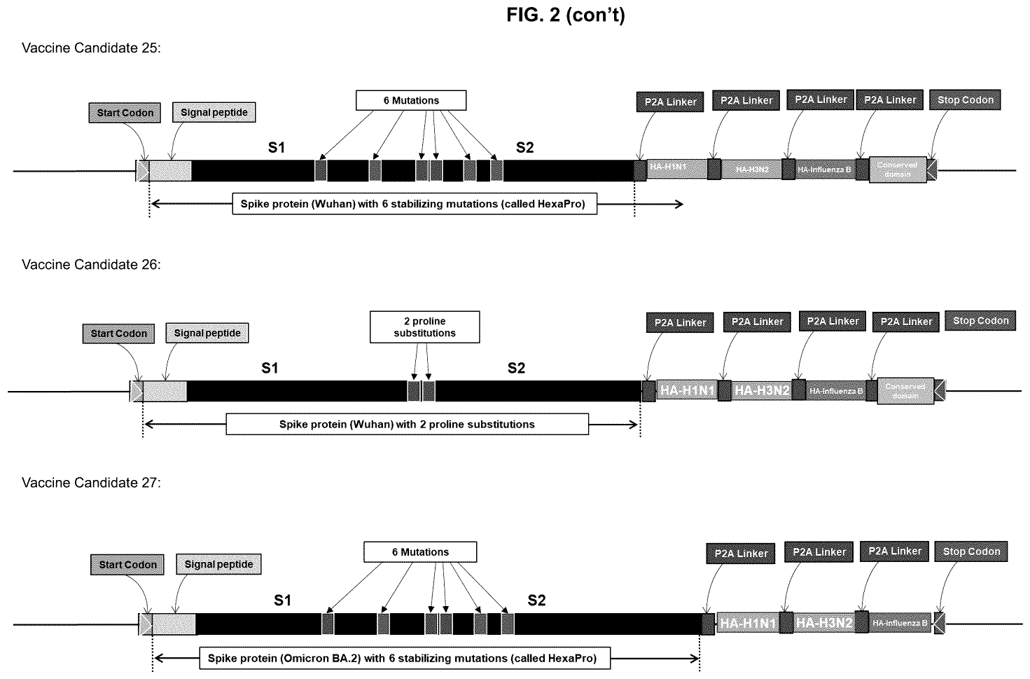

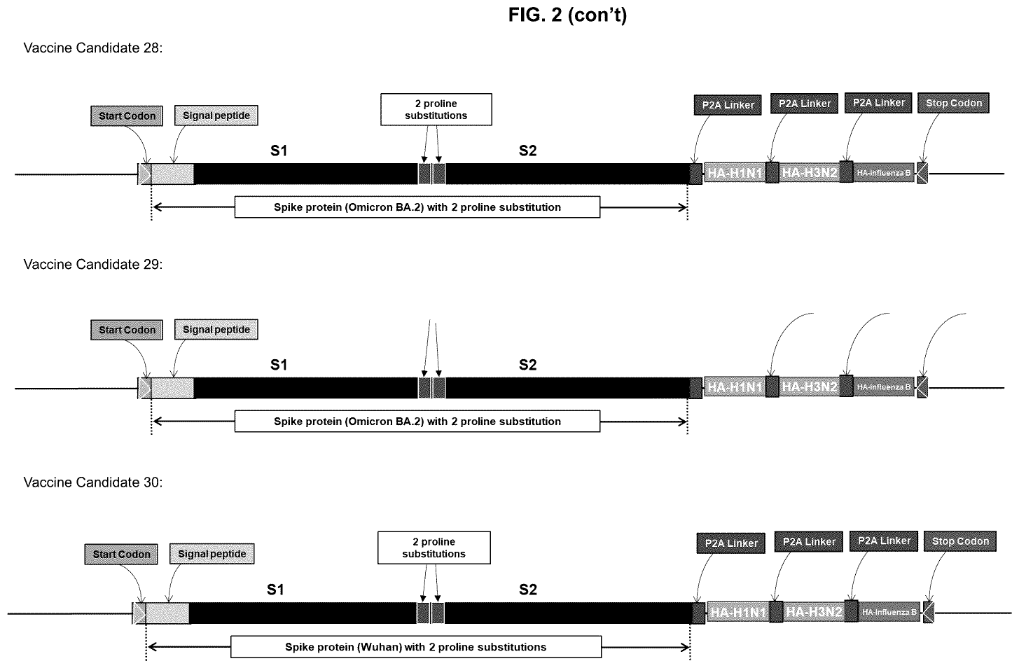

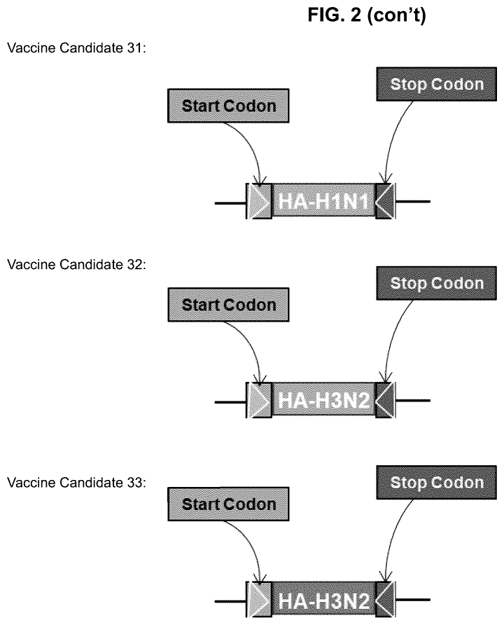

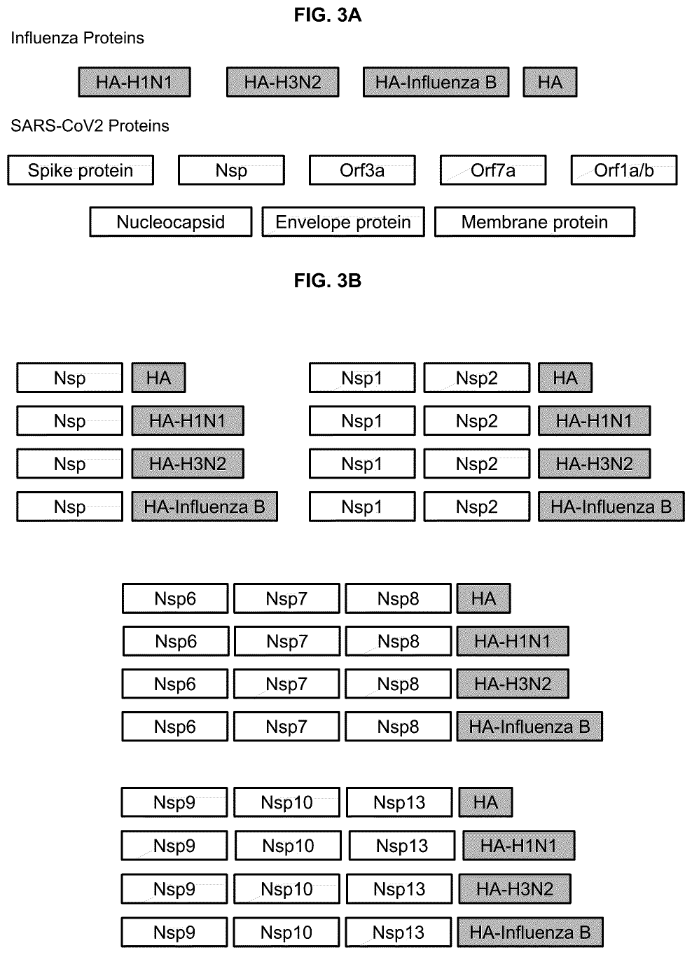

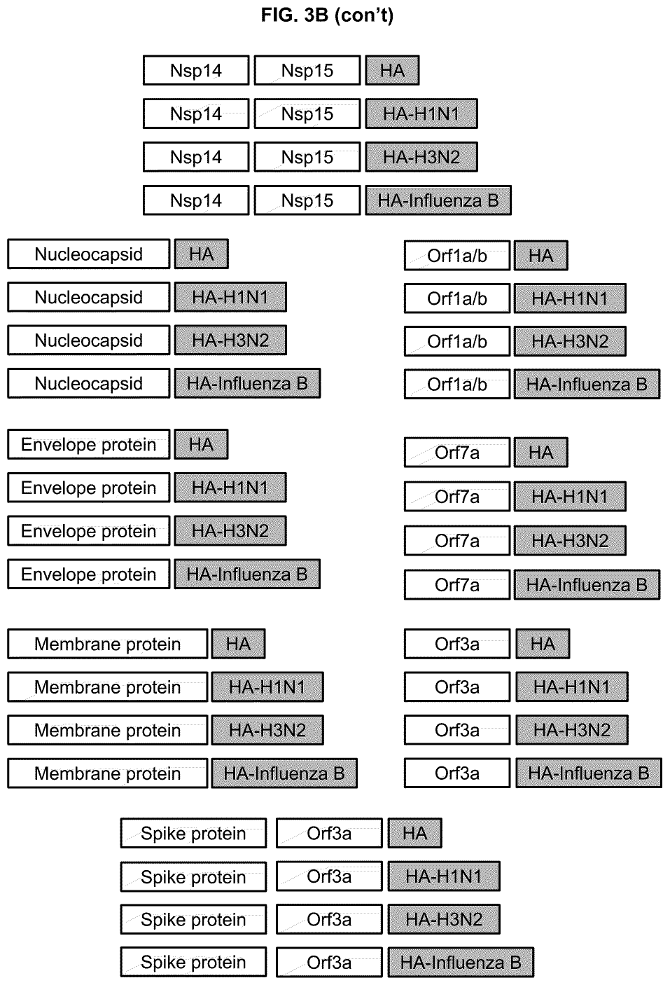

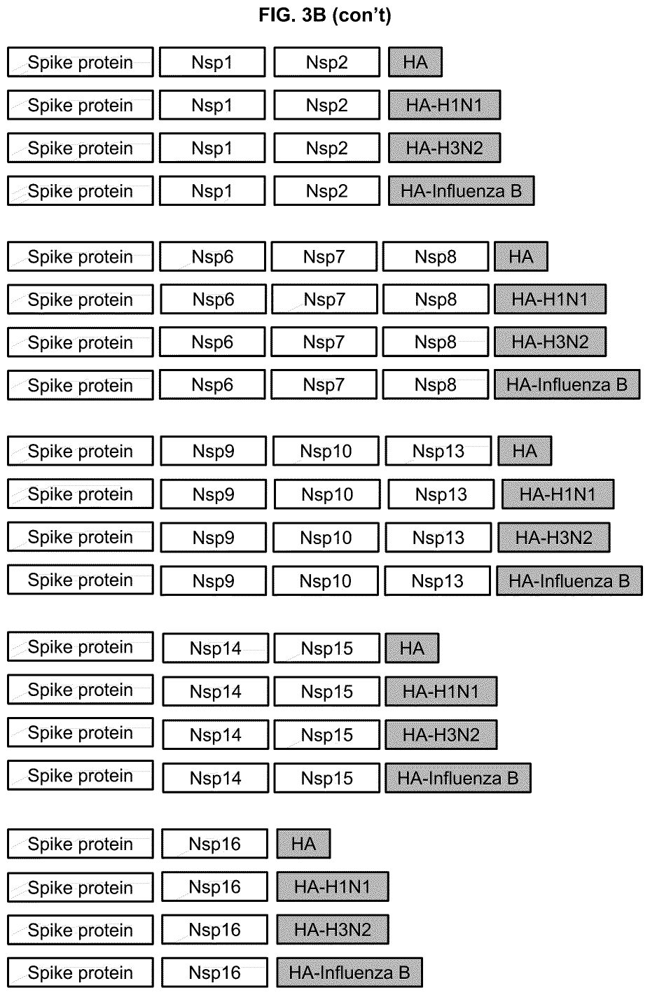

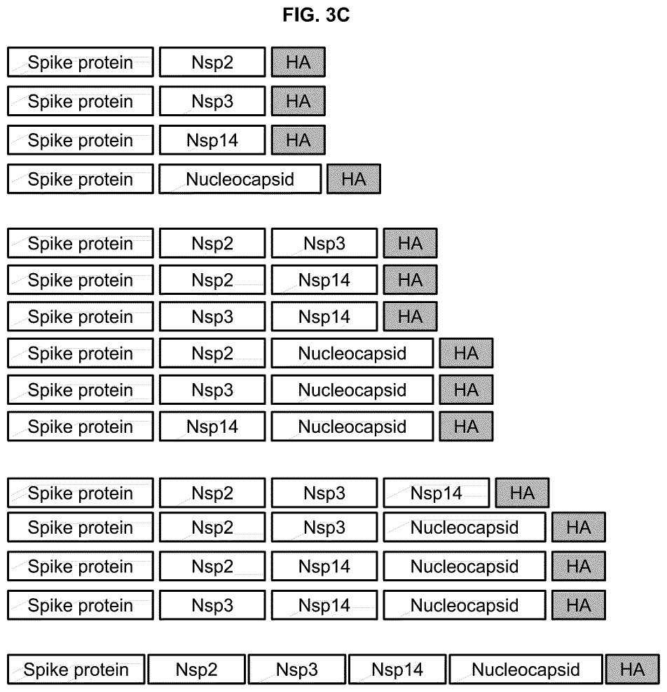

Referring now to FIG. 1, FIG. 2, FIG. 3A, FIG. 3B and FIG. 3C, the present invention features a pan-coronavirus-influenza vaccine composition. The composition comprises at least a portion of a coronavirus spike(S) protein and at least a portion of an influenza hemagglutinin (HA) protein.

### Coronavirus Antigens/Large Coronavirus Sequences:

Coronaviruses used for determining conserved large sequences may include human SARS-CoVs as well as animal CoVs (e.g., bats, pangolins, civet cats, minks, camels, etc.) as described herein. As an example, an evolutionary comparison of genome sequences among beta-coronavirus strains isolated from humans and animals.

Additionally, other coronaviruses may be used for determining conserved large sequences (including human SARS-CoVs as well as animal CoVs (e.g., bats, pangolins, civet cats, minks, camels, etc.)) that meet the criteria to be classified as “variants of concern” or “variants of interest.” Coronavirus variants that appear to meet one or more of the undermentioned criteria may be labeled “variants of interest” or “variants under investigation” pending verification and validation of these properties. In some embodiments, the criteria may include increased transmissibility, increased morbidity, increased mortality, increased risk of “long COVID,” ability to evade detection by diagnostic tests, decreased susceptibility to antiviral drugs (if and when such drugs are available), decreased susceptibility to neutralizing antibodies, either therapeutic (e.g., convalescent plasma or monoclonal antibodies) or in laboratory experiments, ability to evade natural immunity (e.g., causing reinfections), ability to infect vaccinated individuals, increased risk of particular conditions such as multisystem inflammatory syndrome or long-haul COVID or Increased affinity for particular demographic or clinical groups, such as children or immunocompromised individuals. Once validated, variants of interest are renamed “variants of concern” by monitoring organizations, such as the CDC.

In order to identify the conserved large sequences, sequence alignments and analysis were performed as described herein as well as below.

Sequence homology analysis was performed, and the Severe Acute Respiratory Syndrome Coronavirus 2 (SARS-CoV-2) isolate Wuhan-Hu-1 was compared to complete genome with sequences of SARS-CoV-2 variants, common cold coronavirus strains (HKU1 genotype B, CoV-OC43, CoV-NL63, and CoV-229E), SARS-CoV-Urbani, MERS and coronavirus strains from bats (Rhinolophus affinis and R. malayanus), pangolin (Manis javanica), civet cats (Paguma larvata), and camel (Camelus dromedarius and C. bactrianus).

The human SARS-CoV-2 variant genome sequences were retrieved from the GISAID database, representing major Variants of Concern which are known for their high degree of transmissibility and pathogenicity. The sequences used in this study are 20A.EU1 from Spain (EPI_ISL_691726-hCoV-19-VOC-20A.EU1), 20A.EU2 from Australia (EPI_ISL_418799-hCoV-19-VOC-20A.EU2), B.1.1.7 from England (EPI_ISL_581117-hCoV-19-VOC-B.1.1.7), B.1.351 from South Africa (EPI_ISL_660615-hCoV-19-VOC-B.1.351), P.1 from Brazil (EPI_ISL_581117-hCoV-19-VOC-P.1), CAL.20C from California (EPI_ISL_730092-hCoV-19-VOC-B.1.427/B.1.429), B.1.258 from Scotland (EPI_ISL_858559-hCoV-19-VOC-B.1.258), B.1.221 from Belgium/Netherlands (EPI_ISL_734790-hCoV-19-VOC-B.1.221), B.1.367 from Norway/France (EPI_ISL_541518-hCoV-19-VOC-B.1.367), B.1.1.277 from Netherlands/Denmark/UK (EPI_ISL_500783-hCoV-19-VOC-B.1.1.277), B.1.1.302 from Sweden (EPI_ISL_717929-hCoV-19-VOC-B.1.1.302). Similarly, HKU1 genotype B (AY884001), CoV-OC43 (KF923903), CoV-NL63 (NC_005831), and CoV-229E (KY983587), SARS-CoV-Urbani (AY278741.1), MERS (NC_019843).

Bat CoV strains used in this analysis include strains RaTG13 (MN996532.2), Rs672/2006 (FJ588686.1), YNLF_31C (KP886808.1), WIV1 (KF367457.1), WIV16 (KT444582.1), ZXC21 (MG772934.1), RmYN02 (EPI_ISL_412977), bat-RmYN01 (EPI_ISL_412976), MERS-Bat-CoV/P.khulii/Italy/206645-63/2011 (MG596803.1). More-so, five genome sequences representing Pangolin (MT040333.1-PCOV_GX-P4L, MT040334.1-PCOV_GX-P1E, MT040335.1-PCOV_GX-P5L, MT040336.1-PCOV_GX-P5E, MT072864.1-PCOV_GX-P2V, MT121216.1-PCoV-MP789), three Civet cat specific genome sequences (AY572034.1, AY686864.1, AY686863.1), and four COV sequences from camels (NC_028752.1, KF917527.1, MN514967.1, KT368891.1) were included in this sequence homology analysis aimed at evaluating the most conserved regions in different structural and non-structural proteins in CoV genome. These sequences were obtained either from National Center for Biotechnology Information (NCBI) or Global initiative on sharing all influenza data (GISAID). For phylogenetic analyses, SARS-CoV-2 full-genome sequences were aligned with CLUSTAL W using MEGAX. All the SARS-CoV-2 sequences were compared to existing genomes using online NCBI BLAST.

In some embodiments, the large sequences are each highly conserved among one or a combination of: SARS-CoV-2 human strains, SL-CoVs isolated from bats, SL-CoVs isolated from pangolin, SL-CoVs isolated from civet cats, and MERS strains isolated from camels. For example, in certain embodiments, the large sequences are each highly conserved among one or a combination of: at least 50,000 SARS-CoV-2 human strains, five SL-CoVs isolated from bats, five SL-CoVs isolated from pangolin, three SL-CoVs isolated from civet cats, and four MERS strains isolated from camels. In certain embodiments, the large sequences are each highly conserved among one or a combination of: at least 80,000 SARS-CoV-2 human strains, five SL-CoVs isolated from bats, five SL-CoVs isolated from pangolin, three SL-CoVs isolated from civet cats, and four MERS strains isolated from camels. In certain embodiments, the large sequences are each highly conserved among one or a combination of: at least 50,000 SARS-CoV-2 human strains in circulation during the COVID-19 pandemic, at least one CoV that caused a previous human outbreak, five SL-CoVs isolated from bats, five SL-CoVs isolated from pangolin, three SL-CoVs isolated from civet cats, and four MERS strains isolated from camels. In certain embodiments, the large sequences are each highly conserved among at least 1 SARS-CoV-2 human strain in current circulation, at least one CoV that has caused a previous human outbreak, at least one SL-CoV isolated from bats, at least one SL-CoV isolated from pangolin, at least one SL-CoV isolated from civet cats, and at least one MERS strain isolated from camels. In certain embodiments, the large sequences are each highly conserved among at least 1,000 SARS-CoV-2 human strains in current circulation, at least two CoVs that has caused a previous human outbreak, at least two SL-CoVs isolated from bats, at least two SL-CoVs isolated from pangolin, at least two SL-CoVs isolated from civet cats, and at least two MERS strains isolated from camels. In certain embodiments, the large sequences are each highly conserved among one or a combination of: at least one SARS-CoV-2 human strain in current circulation, at least one CoV that has caused a previous human outbreak, at least one SL-CoV isolated from bats, at least one SL-CoV isolated from pangolin, at least one SL-CoV isolated from civet cats, and at least one MERS strain isolated from camels. The present invention is not limited to the aforementioned coronavirus strains that may be used to identify conserved large sequences.

In certain embodiments, one or more of the conserved large sequences are derived from one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; and/or one or more coronaviruses that cause the common cold; or one or more coronaviruses that case past, current, and future outbreaks. SARS-CoV-2 human strains and variants in current circulation may include the original SARS-CoV-2 strain (SARS-CoV-2 isolate Wuhan-Hu-1), and several variants of SARS-CoV-2 including but not limited to variant B.1.177 (Spain); variant B.1.160 (Australia), variant B.1.1.7 (UK), variant P.1 (Japan/Brazil), variant B.1.351 (South Africa), variant B.1.427 (California), variant B.1.429 (California), variant B.1.258 (Scotland); variant B.1.221 (Belgium/Netherlands); variant B.1.367 (Norway/France); variant B.1.1.277 (UK); variant B.1.1.302 (Sweden); variant B.1.525 (North America, Europe, Asia, Africa, and Australia); variant B.1.526 (New York), variant S: 677H; variant S: 677P; B.1.617.2-Delta, variant B.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5). The present invention is not limited to the aforementioned variants of SARS-CoV-2 and encompasses variants identified in the future. The one or more coronaviruses that cause the common cold may include but are not limited to strains 229E (alpha coronavirus), NL63 (alpha coronavirus), OC43 (beta coronavirus), HKU1 (beta coronavirus).

The antigen may comprise large sequences, such as conserved large sequences that are highly conserved among human and animal coronaviruses. As used herein, the term large sequence refers to a sequence having at least 25 amino acids or at least 75 nucleotides. The large sequences comprise epitopes, such as the conserved epitopes described herein.

In some embodiments, the large sequence has at least 75 nt. In some embodiments, the large sequence has at least 150 nt. In some embodiments, the large sequence has at least 200 nt. In some embodiments, the large sequence has at least 250 nt. In some embodiments, the large sequence has at least 300 nt. In some embodiments, the large sequence has at least 400 nt. In some embodiments, the large sequence has at least 500 nt. In some embodiments, the large sequence has at least 600 nt. In some embodiments, the large sequence has at least 700 nt. In some embodiments, the large sequence has at least 800 nt. In some embodiments, the large sequence has at least 900 nt. In some embodiments, the large sequence has at least 1000 nt. In some embodiments, the large sequence has at least 1500 nt. In some embodiments, the large sequence has at least 2000 nt.

The large sequence(s) may be derived from structural proteins, non-structural proteins, or a combination thereof. The large sequence(s) may be selected from ORF1ab protein, Spike glycoprotein (e.g., the RBD), ORF3a protein, Envelope protein, Membrane glycoprotein, ORF6 protein, ORF7a protein, ORF7b protein, ORF8 protein, Nucleocapsid protein, and/or an ORF10 protein. Note the ORF1ab protein comprises nonstructural protein (Nsp) 1, Nsp2, Nsp3, Nsp4, Nsp5, Nsp6, Nsp7, Nsp8, Nsp9, Nsp10, Nsp11, Nsp12, Nsp13, Nsp14, Nsp15 and Nsp16.

In some embodiments, the portion of the coronavirus spike(S) protein is highly conserved among human and animal coronaviruses. The portion of the coronavirus spike(S) protein may be derived from one or more of: one or more SARS-CoV-2 human strains or variants in current circulation; one or more coronaviruses that have caused a previous human outbreak; one or more coronaviruses isolated from animals selected from a group consisting of bats, pangolins, civet cats, minks, camels, and other animals receptive to coronaviruses; or one or more coronaviruses that cause the common cold; or one or more coronaviruses that cause past, current, and future outbreaks.

In some embodiments, the conserved large sequences are identified by: performing a sequence alignment and analysis of a particular number of coronavirus or influenza sequences to determine sequence similarity or identity amongst the group of analyzed sequences. The conserved large sequences are those that are among the most highly conserved sequences identified in the analysis.

In some embodiments, the one or more SARS-CoV-2 human strains or variants in current circulation are selected from: variant B.1.177; variant B.1.160, variant B.1.1.7; variant B.1.351; variant P.1;

variant B.1.427/B.1.429; variant B.1.258; variant B.1.221; variant B.1.367; variant B.1.1.277; variant B.1.1.302; variant B.1.525; variant B.1.526, variant S: 677H; variant S: 677P; variant B.1.1.529-Omicron (BA.1); variant B.1.1.529-Omicron (BA.2); and variant B.1.617.2-Delta). In some embodiments, the one or more coronaviruses that cause the common cold are selected from: 229E alpha coronavirus, NL63 alpha coronavirus, OC43 beta coronavirus, and HKU1 beta coronavirus.

In some embodiments, the portion of the coronavirus spike(S) protein is derived from a full-length spike glycoprotein. In other embodiments, the portion of the coronavirus spike(S) protein is derived from a partial spike glycoprotein. In some embodiments, the portion of the coronavirus spike(S) protein comprises at least one proline substitution, or at least two proline substitution, or at least, four proline substitutions, or at least six proline substitutions. The portion of the coronavirus spike(S) protein may comprise two consecutive proline substitutions at amino acid positions 986 and 987. The proline substitutions may comprise K986P and V987P mutations. In further embodiments, the portion of the coronavirus spike(S) protein is the receptor-binding domain (RBD). In some embodiments, the RBD comprises a trimerized SARS-CoV-2 receptor-binding domain (RBD).

In some embodiments, the transmembrane anchor of the spike protein has an intact S1-S2 cleavage site. In some embodiments, the spike protein is in its stabilized conformation. In some embodiments, the spike protein is stabilized with proline substitutions at amino acid positions 986 and 987 at the top of the central helix in the S2 subunit. In some embodiments, the composition comprises a trimerized SARS-CoV-2 receptor-binding domain (RBD). In some embodiments, the trimerized SARS-CoV-2 receptor-binding domain (RBD) sequence is modified by the addition of a T4 fibritin-derived foldon trimerization domain. In some embodiments, the addition of a T4 fibritin-derived foldon trimerization domain increases immunogenicity by multivalent display

Table 1: Shows non-limiting examples of a portion of a coronavirus spike(S) protein that may be used in accordance with the present invention.

| | Sequence | SEQ ID NO: |
|---|---|---|
| Spike protein | AACCTGACCACCAGGACCCAGCTGCCCCCCGCCTACACCAACAGCTT | 1 |
| (Wuhan) with | CACCAGGGGCGTGTACTACCCCGACAAGGTGTTCAGGAGCAGCGTGC | |
| six proline | TGCACAGCACCCAGGACCTGTTCCTGCCCTTCTTCAGCAACGTGACCT | |
| substitutions | GGTTCCACGCCATCCACGTGAGCGGCACCAACGGCACCAAGAGGTTC | |
| (F817P, | GACAACCCCGTGCTGCCCTTCAACGACGGCGTGTACTTCGCCAGCAC | |
| A892P, A899P, | CGAGAAGAGCAACATCATCAGGGGCTGGATCTTCGGCACCACCCTGG | |
| A942P, K986P, | ACAGCAAGACCCAGAGCCTGCTGATCGTGAACAACGCCACCAACGTG | |
| V987P) | GTGATCAAGGTGTGCGAGTTCCAGTTCTGCAACGACCCCTTCCTGGGC | |
| | GTGTACTACCACAAGAACAACAAGAGCTGGATGGAGAGCGAGTTCAG | |
| | GGTGTACAGCAGCGCCAACAACTGCACCTTCGAGTACGTGAGCCAGC | |
| | CCTTCCTGATGGACCTGGAGGGCAAGCAGGGCAACTTCAAGAACCTG | |
| | AGGGAGTTCGTGTTCAAGAACATCGACGGCTACTTCAAGATCTACAGC | |
| | AAGCACACCCCCATCAACCTGGTGAGGGACCTGCCCCAGGGCTTCAG | |
| | CGCCCTGGAGCCCCTGGTGGACCTGCCCATCGGCATCAACATCACCA | |
| | GGTTCCAGACCCTGCTGGCCCTGCACAGGAGCTACCTGACCCCCGGC | |
| | GACAGCAGCAGCGGCTGGACCGCCGGCGCCGCCGCCTACTACGTGG | |
| | GCTACCTGCAGCCCAGGACCTTCCTGCTGAAGTACAACGAGAACGGC | |
| | ACCATCACCGACGCCGTGGACTGCGCCCTGGACCCCCTGAGCGAGAC | |
| | CAAGTGCACCCTGAAGAGCTTCACCGTGGAGAAGGGCATCTACCAGA | |
| | CCAGCAACTTCAGGGTGCAGCCCACCGAGAGCATCGTGAGGTTCCCC | |
| | AACATCACCAACCTGTGCCCCTTCGGCGAGGTGTTCAACGCCACCAG | |
| | GTTCGCCAGCGTGTACGCCTGGAACAGGAAGAGGATCAGCAACTGCG | |
| | TGGCCGACTACAGCGTGCTGTACAACAGCGCCAGCTTCAGCACCTTCA | |
| | AGTGCTACGGCGTGAGCCCCACCAAGCTGAACGACCTGTGCTTCACC | |
| | AACGTGTACGCCGACAGCTTCGTGATCAGGGGCGACGAGGTGAGGCA | |
| | GATCGCCCCCGGCCAGACCGGCAAGATCGCCGACTACAACTACAAGC | |
| | TGCCCGACGACTTCACCGGCTGCGTGATCGCCTGGAACAGCAACAAC | |
| | CTGGACAGCAAGGTGGGGGGCAACTACAACTACCTGTACAGGCTGTT | |
| | CAGGAAGAGCAACCTGAAGCCCTTCGAGAGGGACATCAGCACCGAGA | |
| | TCTACCAGGCCGGCAGCACCCCCTGCAACGGCGTGGAGGGCTTCAAC | |
| | TGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGC | |
| | ACGCCCCCGCCACCGTGTGCGGCCCCAAGAAGAGCACCAACCTGGTG | |
| | AAGAACAAGTGCGTGAACTTCAACTTCAACGGCCTGACCGGCACCGG | |
| | CGTGCTGACCGAGAGCAACAAGAAGTTCCTGCCCTTCCAGCAGTTCG | |
| | GCAGGGACATCGCCGACACCACCGACGCCGTGAGGGACCCCCAGAC | |
| | CCTGGAGATCCTGGACATCACCCCCTGCAGCTTCGGCGGCGTGAGCG | |
| | TGATCACCCCCGGCACCAACACCAGCAACCAGGTGGCCGTGCTGTAC | |
| | CAGGACGTGAACTGCACCGAGGTGCCCGTGGCCATCCACGCCGACCA | |
| | GCTGACCCCCACCTGGAGGGTGTACAGCACCGGCAGCAACGTGTTCC | |
| | AGACCAGGGCCGGCTGCCTGATCGGCGCCGAGCACGTGAACAACAG | |
| | CTACGAGTGCGACATCCCCATCGGCGCCGGCATCTGCGCCAGCTACC | |
| | AGACCCAGACCAACAGCCCCGGCAGCGCCAGCAGCGTGGCCAGCCA | |
| | GAGCATCATCGCCTACACCATGAGCCTGGGCGCCGAGAACAGCGTGG | |
| | CCTACAGCAACAACAGCATCGCCATCCCCACCAACTTCACCATCAGCG | |
| | TGACCACCGAGATCCTGCCCGTGAGCATGACCAAGACCAGCGTGGAC | |
| | TGCACCATGTACATCTGCGGCGACAGCACCGAGTGCAGCAACCTGCT | |
| | GCTGCAGTACGGCAGCTTCTGCACCCAGCTGAACAGGGCCCTGACCG | |
| | GCATCGCCGTGGAGCAGGACAAGAACACCCAGGAGGTGTTCGCCCAG | |
| | GTGAAGCAGATCTACAAGACCCCCCCCATCAAGGACTTCGGCGGCTTC | |
| | AACTTCAGCCAGATCCTGCCCGACCCCAGCAAGCCCAGCAAGAGGAG | |
| | CCCCATCGAGGACCTGCTGTTCAACAAGGTGACCCTGGCCGACGCCG | |
| | GCTTCATCAAGCAGTACGGCGACTGCCTGGGCGACATCGCCGCCAGG | |
| | GACCTGATCTGCGCCCAGAAGTTCAACGGCCTGACCGTGCTGCCCCC | |
| | CCTGCTGACCGACGAGATGATCGCCCAGTACACCAGCGCCCTGCTGG | |
| | CCGGCACCATCACCAGCGGCTGGACCTTCGGCGCCGGCCCCGCCCT | |
| | GCAGATCCCCTTCCCCATGCAGATGGCCTACAGGTTCAACGGCATCG | |
| | GCGTGACCCAGAACGTGCTGTACGAGAACCAGAAGCTGATCGCCAAC | |
| | CAGTTCAACAGCGCCATCGGCAAGATCCAGGACAGCCTGAGCAGCAC | |
| | CCCCAGCGCCCTGGGCAAGCTGCAGGACGTGGTGAACCAGAACGCC | |
| | CAGGCCCTGAACACCCTGGTGAAGCAGCTGAGCAGCAACTTCGGCGC | |
| | CATCAGCAGCGTGCTGAACGACATCCTGAGCAGGCTGGACCCCCCCG | |
| | AGGCCGAGGTGCAGATCGACAGGCTGATCACCGGCAGGCTGCAGAG | |
| | CCTGCAGACCTACGTGACCCAGCAGCTGATCAGGGCCGCCGAGATCA | |
| | GGGCCAGCGCCAACCTGGCCGCCACCAAGATGAGCGAGTGCGTGCT | |
| | GGGCCAGAGCAAGAGGGTGGACTTCTGCGGCAAGGGCTACCACCTGA | |
| | TGAGCTTCCCCCAGAGCGCCCCCCACGGCGTGGTGTTCCTGCACGTG | |
| | ACCTACGTGCCCGCCCAGGAGAAGAACTTCACCACCGCCCCCGCCAT | |
| | CTGCCACGACGGCAAGGCCCACTTCCCCAGGGAGGGCGTGTTCGTGA | |
| | GCAACGGCACCCACTGGTTCGTGACCCAGAGGAACTTCTACGAGCCC | |
| | CAGATCATCACCACCGACAACACCTTCGTGAGCGGCAACTGCGACGT | |
| | GGTGATCGGCATCGTGAACAACACCGTGTACGACCCCCTGCAGCCCG | |
| | AGCTGGACAGCTTCAAGGAGGAGCTGGACAAGTACTTCAAGAACCACA | |
| | CCAGCCCCGACGTGGACCTGGGCGACATCAGCGGCATCAACGCCAGC | |
| | GTGGTGAACATCCAGAAGGAGATCGACAGGCTGAACGAGGTGGCCAA | |
| | GAACCTGAACGAGAGCCTGATCGACCTGCAGGAGCTGGGCAAGTACG | |
| | AGCAGTACATCAAGTGGCCCTGGTACATCTGGCTGGGCTTCATCGCCG | |
| | GCCTGATCGCCATCGTGATGGTGACCATCATGCTGTGCTGCATGACCA | |
| | GCTGCTGCAGCTGCCTGAAGGGCTGCTGCAGCTGCGGCAGCTGCTGC | |
| | AAGTTCGACGAGGACGACAGCGAGCCCGTGCTGAAGGGCGTGAAGCT | |
| | GCACTACACC | |
| spike | MFVFLVLLPLVSSQCVNLTTRTQLPPAYTNSFTRGVYYPDKVFRSSVLHST | 2 |
| glycoprotein | QDLFLPFFSNVTWFHAIHVSGTNGTKRFDNPVLPFNDGVYFASTEKSNIIR | |
| with six proline | GWIFGTTLDSKTQSLLIVNNATNVVIKVCEFQFCNDPFLGVYYHKNNKSW | |
| substitutions | MESEFRVYSSANNCTFEYVSQPFLMDLEGKQGNFKNLREFVFKNIDGYFK | |
| (F817P, | IYSKHTPINLVRDLPQGFSALEPLVDLPIGINITRFQTLLALHRSYLTPGDSS | |
| A892P, A899P, | SGWTAGAAAYYVGYLQPRTFLLKYNENGTITDAVDCALDPLSETKCTLKS | |
| A942P, K986P, | FTVEKGIYQTSNFRVQPTESIVRFPNITNLCPFGEVFNATRFASVYAWNRK | |
| V987P) | RISNCVADYSVLYNSASFSTFKCYGVSPTKLNDLCFTNVYADSFVIRGDEV | |
| | RQIAPGQTGKIADYNYKLPDDFTGCVIAWNSNNLDSKVGGNYNYLYRLFR | |
| | KSNLKPFERDISTEIYQAGSTPCNGVEGFNCYFPLQSYGFQPTNGVGYQP | |
| | YRVVVLSFELLHAPATVCGPKKSTNLVKNKCVNFNFNGLTGTGVLTESNK | |
| | KFLPFQQFGRDIADTTDAVTRAGCLIGAEHVNNSYECDIPIGAGICASYQT | |
| | QTNRDPQTLEILDITPCSFGGVSVITPGTNTSNQVAVLYQDVNCTEVPVAI | |
| | HADQLTPTWRVYSTGSNVFQSPRRARSVASQSIIAYTMSLGAENSVAYSN | |
| | NSIAIPTNFTISVTTEILPVSMTKTSVDCTMYICGDSTECSNLLLQYGSFCTQ | |
| | LNRALTGIAVEQDKNTQEVFAQVKQIYKTPPIKDFGGFNFSQILPDPSKPSK | |
| | RSPIEDLLFNKVTLADAGFIKQYGDCLGDIAARDLICAQKFNGLTVLPPLLT | |
| | DEMIAQYTSALLAGTITSGWTFGAGPALQIPFPMQMAYRFNGIGVTQNVLY | |
| | ENQKLIANQFNSAIGKIQDSLSSTPSALGKLQDVVNQNAQALNTLVKQLSS | |
| | NFGAISSVLNDILSRLDPPEAEVQIDRLITGRLQSLQTYVTQQLIRAAEIRAS | |
| | ANLAATKMSECVLGQSKRVDFCGKGYHLMSFPQSAPHGVVFLHVTYVPA | |
| | QEKNFTTAPAICHDGKAHFPREGVFVSNGTHWFVTQRNFYEPQIITTDNTF | |
| | VSGNCDVVIGIVNNTVYDPLQPELDSFKEELDKYFKNHTSPDVDLGDISGI | |
| | NASVVNIQKEIDRLNEVAKNLNESLIDLQELGKYEQYIKWPWYIWLGFIAGL | |
| | IAIVMVTIMLCCMTSCCSCLKGCCSCGSCCKFDEDDSEPVLKGVKLHYT | |
| Spike protein | AACCTGACCACCAGGACCCAGCTGCCCCCCGCCTACACCAACAGCTT | 3 |
| (Wuhan) with | CACCAGGGGCGTGTACTACCCCGACAAGGTGTTCAGGAGCAGCGTGC | |
| two proline | TGCACAGCACCCAGGACCTGTTCCTGCCCTTCTTCAGCAACGTGACCT | |
| substitutions | GGTTCCACGCCATCCACGTGAGCGGCACCAACGGCACCAAGAGGTTC | |
| (K986P, | GACAACCCCGTGCTGCCCTTCAACGACGGCGTGTACTTCGCCAGCAC | |
| V987P) | CGAGAAGAGCAACATCATCAGGGGCTGGATCTTCGGCACCACCCTGG | |
| | ACAGCAAGACCCAGAGCCTGCTGATCGTGAACAACGCCACCAACGTG | |
| | GTGATCAAGGTGTGCGAGTTCCAGTTCTGCAACGACCCCTTCCTGGGC | |
| | GTGTACTACCACAAGAACAACAAGAGCTGGATGGAGAGCGAGTTCAG | |
| | GGTGTACAGCAGCGCCAACAACTGCACCTTCGAGTACGTGAGCCAGC | |
| | CCTTCCTGATGGACCTGGAGGGCAAGCAGGGCAACTTCAAGAACCTG | |
| | AGGGAGTTCGTGTTCAAGAACATCGACGGCTACTTCAAGATCTACAGC | |
| | AAGCACACCCCCATCAACCTGGTGAGGGACCTGCCCCAGGGCTTCAG | |
| | CGCCCTGGAGCCCCTGGTGGACCTGCCCATCGGCATCAACATCACCA | |
| | GGTTCCAGACCCTGCTGGCCCTGCACAGGAGCTACCTGACCCCCGGC | |
| | GACAGCAGCAGCGGCTGGACCGCCGGCGCCGCCGCCTACTACGTGG | |
| | GCTACCTGCAGCCCAGGACCTTCCTGCTGAAGTACAACGAGAACGGC | |
| | ACCATCACCGACGCCGTGGACTGCGCCCTGGACCCCCTGAGCGAGAC | |
| | CAAGTGCACCCTGAAGAGCTTCACCGTGGAGAAGGGCATCTACCAGA | |
| | CCAGCAACTTCAGGGTGCAGCCCACCGAGAGCATCGTGAGGTTCCCC | |
| | AACATCACCAACCTGTGCCCCTTCGGCGAGGTGTTCAACGCCACCAG | |
| | GTTCGCCAGCGTGTACGCCTGGAACAGGAAGAGGATCAGCAACTGCG | |
| | TGGCCGACTACAGCGTGCTGTACAACAGCGCCAGCTTCAGCACCTTCA | |
| | AGTGCTACGGCGTGAGCCCCACCAAGCTGAACGACCTGTGCTTCACC | |
| | AACGTGTACGCCGACAGCTTCGTGATCAGGGGCGACGAGGTGAGGCA | |
| | GATCGCCCCCGGCCAGACCGGCAAGATCGCCGACTACAACTACAAGC | |
| | TGCCCGACGACTTCACCGGCTGCGTGATCGCCTGGAACAGCAACAAC | |
| | CTGGACAGCAAGGTGGGCGGCAACTACAACTACCTGTACAGGCTGTT | |
| | CAGGAAGAGCAACCTGAAGCCCTTCGAGAGGGACATCAGCACCGAGA | |
| | TCTACCAGGCCGGCAGCACCCCCTGCAACGGCGTGGAGGGCTTCAAC | |
| | TGCTACTTCCCCCTGCAGAGCTACGGCTTCCAGCCCACCAACGGCGT | |
| | GGGCTACCAGCCCTACAGGGTGGTGGTGCTGAGCTTCGAGCTGCTGC | |
| | ACGCCCCCGCCACCGTGTGCGGCCCCAAGAAGAGCACCAACCTGGTG | |
| | AAGAACAAGTGCGTGAACTTCAACTTCAACGGCCTGACCGGCACCGG | |
| | CGTGCTGACCGAGAGCAACAAGAAGTTCCTGCCCTTCCAGCAGTTCG | |
| | GCAGGGACATCGCCGACACCACCGACGCCGTGAGGGACCCCCAGAC | |
| | CCTGGAGATCCTGGACATCACCCCCTGCAGCTTCGGCGGCGTGAGCG | |
| | TGATCACCCCCGGCACCAACACCAGCAACCAGGTGGCCGTGCTGTAC | |
| | CAGGACGTGAACTGCACCGAGGTGCCCGTGGCCATCCACGCCGACCA | |
| | GCTGACCCCCACCTGGAGGGTGTACAGCACCGGCAGCAACGTGTTCC | |
| | AGACCAGGGCCGGCTGCCTGATCGGCGCCGAGCACGTGAACAACAG | |
| | CTACGAGTGCGACATCCCCATCGGCGCCGGCATCTGCGCCAGCTACC | |
| | AGACCCAGACCAACAGCCCCAGGAGGGCCAGGAGCGTGGCCAGCCA | |
| | GAGCATCATCGCCTACACCATGAGCCTGGGCGCCGAGAACAGCGTGG | |
| | CCTACAGCAACAACAGCATCGCCATCCCCACCAACTTCACCATCAGCG | |
| | TGACCACCGAGATCCTGCCCGTGAGCATGACCAAGACCAGCGTGGAC | |
| | TGCACCATGTACATCTGCGGCGACAGCACCGAGTGCAGCAACCTGCT | |
| | GCTGCAGTACGGCAGCTTCTGCACCCAGCTGAACAGGGCCCTGACCG | |
| | GCATCGCCGTGGAGCAGGACAAGAACACCCAGGAGGTGTTCGCCCAG | |
| | GTGAAGCAGATCTACAAGACCCCCCCCATCAAGGACTTCGGCGGCTTC | |
| | AACTTCAGCCAGATCCTGCCCGACCCCAGCAAGCCCAGCAAGAGGAG | |
| | CTTCATCGAGGACCTGCTGTTCAACAAGGTGACCCTGGCCGACGCCG | |
| | GCTTCATCAAGCAGTACGGCGACTGCCTGGGCGACATCGCCGCCAGG | |
| | GACCTGATCTGCGCCCAGAAGTTCAACGGCCTGACCGTGCTGCCCCC | |
| | CCTGCTGACCGACGAGATGATCGCCCAGTACACCAGCGCCCTGCTGG | |
| | CCGGCACCATCACCAGCGGCTGGACCTTCGGCGCCGGCGCCGCCCT | |
| | GCAGATCCCCTTCGCCATGCAGATGGCCTACAGGTTCAACGGCATCG | |
| | GCGTGACCCAGAACGTGCTGTACGAGAACCAGAAGCTGATCGCCAAC | |
| | CAGTTCAACAGCGCCATCGGCAAGATCCAGGACAGCCTGAGCAGCAC | |
| | CGCCAGCGCCCTGGGCAAGCTGCAGGACGTGGTGAACCAGAACGCC | |
| | CAGGCCCTGAACACCCTGGTGAAGCAGCTGAGCAGCAACTTCGGCGC | |
| | CATCAGCAGCGTGCTGAACGACATCCTGAGCAGGCTGGACCCCCCCG | |
| | AGGCCGAGGTGCAGATCGACAGGCTGATCACCGGCAGGCTGCAGAG | |
| | CCTGCAGACCTACGTGACCCAGCAGCTGATCAGGGCCGCCGAGATCA | |
| | GGGCCAGCGCCAACCTGGCCGCCACCAAGATGAGCGAGTGCGTGCT | |
| | GGGCCAGAGCAAGAGGGTGGACTTCTGCGGCAAGGGCTACCACCTGA | |
| | TGAGCTTCCCCCAGAGCGCCCCCCACGGCGTGGTGTTCCTGCACGTG | |
| | ACCTACGTGCCCGCCCAGGAGAAGAACTTCACCACCGCCCCCGCCAT | |
| | CTGCCACGACGGCAAGGCCCACTTCCCCAGGGAGGGCGTGTTCGTGA | |
| | GCAACGGCACCCACTGGTTCGTGACCCAGAGGAACTTCTACGAGCCC | |
| | CAGATCATCACCACCGACAACACCTTCGTGAGCGGCAACTGCGACGT | |
| | GGTGATCGGCATCGTGAACAACACCGTGTACGACCCCCTGCAGCCCG | |
| | AGCTGGACAGCTTCAAGGAGGAGCTGGACAAGTACTTCAAGAACCACA | |
| | CCAGCCCCGACGTGGACCTGGGCGACATCAGCGGCATCAACGCCAGC | |
| | GTGGTGAACATCCAGAAGGAGATCGACAGGCTGAACGAGGTGGCCAA | |
| | GAACCTGAACGAGAGCCTGATCGACCTGCAGGAGCTGGGCAAGTACG | |
| | AGCAGTACATCAAGTGGCCCTGGTACATCTGGCTGGGCTTCATCGCCG | |
| | GCCTGATCGCCATCGTGATGGTGACCATCATGCTGTGCTGCATGACCA | |
| | GCTGCTGCAGCTGCCTGAAGGGCTGCTGCAGCTGCGGCAGCTGCTGC | |
| | AAGTTCGACGAGGACGACAGCGAGCCCGTGCTGAAGGGCGTGAAGCT | |
| | GCACTACACC | |
| spike | MFVFLVLLPLVSSQCVNLTTRTQLPPAYTNSFTRGVYYPDKVFRSSVLHST | 4 |
| glycoprotein | QDLFLPFFSNVTWFHAIHVSGTNGTKRFDNPVLPFNDGVYFASTEKSNIIR | |
| with two proline | GWIFGTTLDSKTQSLLIVNNATNVVIKVCEFQFCNDPFLGVYYHKNNKSW | |
| substitutions | MESEFRVYSSANNCTFEYVSQPFLMDLEGKQGNFKNLREFVFKNIDGYFK | |
| (K986P, | IYSKHTPINLVRDLPQGFSALEPLVDLPIGINITRFQTLLALHRSYLTPGDSS | |
| V987P) | SGWTAGAAAYYVGYLQPRTFLLKYNENGTITDAVDCALDPLSETKCTLKS | |
| | FTVEKGIYQTSNFRVQPTESIVRFPNITNLCPFGEVFNATRFASVYAWNRK | |
| | RISNCVADYSVLYNSASFSTFKCYGVSPTKLNDLCFTNVYADSFVIRGDEV | |
| | RQIAPGQTGKIADYNYKLPDDFTGCVIAWNSNNLDSKVGGNYNYLYRLFR | |
| | KSNLKPFERDISTEIYQAGSTPCNGVEGFNCYFPLQSYGFQPTNGVGYQP | |
| | YRVVVLSFELLHAPATVCGPKKSTNLVKNKCVNFNFNGLTGTGVLTESNK | |
| | KFLPFQQFGRDIADTTDAVRDPQTLEILDITPCSFGGVSVITPGTNTSNQVA | |
| | VLYQDVNCTEVPVAIHADQLTPTWRVYSTGSNVFQTRAGCLIGAEHVNNS | |
| | YECDIPIGAGICASYQTQTNSPRRARSVASQSIIAYTMSLGAENSVAYSNN | |
| | SIAIPTNFTISVTTEILPVSMTKTSVDCTMYICGDSTECSNLLLQYGSFCTQL | |
| | NRALTGIAVEQDKNTQEVFAQVKQIYKTPPIKDFGGFNFSQILPDPSKPSK | |
| | RSFIEDLLFNKVTLADAGFIKQYGDCLGDIAARDLICAQKFNGLTVLPPLLT | |
| | DEMIAQYTSALLAGTITSGWTFGAGAALQIPFAMQMAYRFNGIGVTQNVLY | |
| | ENQKLIANQFNSAIGKIQDSLSSTASALGKLQDVVNQNAQALNTLVKQLSS | |
| | NFGAISSVLNDILSRLDPPEAEVQIDRLITGRLQSLQTYVTQQLIRAAEIRAS | |
| | ANLAATKMSECVLGQSKRVDFCGKGYHLMSFPQSAPHGVVFLHVTYVPA | |
| | QEKNFTTAPAICHDGKAHFPREGVFVSNGTHWFVTQRNFYEPQIITTDNTF | |
| | VSGNCDVVIGIVNNTVYDPLQPELDSFKEELDKYFKNHTSPDVDLGDISGI | |
| | NASVVNIQKEIDRLNEVAKNLNESLIDLQELGKYEQYIKWPWYIWLGFIAGL | |
| | IAIVMVTIMLCCMTSCCSCLKGCCSCGSCCKFDEDDSEPVLKGVKLHYT | |
| Spike protein | AACCTGATCACCAGGACCCAGAGCTACACCAACAGCTTCACCAGGGG | 5 |
| (Omicron BA.2) | CGTGTACTACCCCGACAAGGTGTTCAGGAGCAGCGTGCTGCACAGCA | |
| with 6 | CCCAGGACCTGTTCCTGCCCTTCTTCAGCAACGTGACCTGGTTCCACG | |
| stabilizing | CCATCCACGTGAGCGGCACCAACGGCACCAAGAGGTTCGACAACCCC | |
| mutations | GTGCTGCCCTTCAACGACGGCGTGTACTTCGCCAGCACCGAGAAGAG | |
| | CAACATCATCAGGGGCTGGATCTTCGGCACCACCCTGGACAGCAAGA | |
| | CCCAGAGCCTGCTGATCGTGAACAACGCCACCAACGTGGTGATCAAG | |
| | GTGTGCGAGTTCCAGTTCTGCAACGACCCCTTCCTGGACGTGTACTAC | |
| | CACAAGAACAACAAGAGCTGGATGGAGAGCGAGTTCAGGGTGTACAG | |
| | CAGCGCCAACAACTGCACCTTCGAGTACGTGAGCCAGCCCTTCCTGAT | |
| | GGACCTGGAGGGCAAGCAGGGCAACTTCAAGAACCTGAGGGAGTTCG | |
| | TGTTCAAGAACATCGACGGCTACTTCAAGATCTACAGCAAGCACACCC | |
| | CCATCAACCTGGGCAGGGACCTGCCCCAGGGCTTCAGCGCCCTGGAG | |
| | CCCCTGGTGGACCTGCCCATCGGCATCAACATCACCAGGTTCCAGAC | |
| | CCTGCTGGCCCTGCACAGGAGCTACCTGACCCCCGGCGACAGCAGCA | |
| | GCGGCTGGACCGCCGGCGCCGCCGCCTACTACGTGGGCTACCTGCA | |
| | GCCCAGGACCTTCCTGCTGAAGTACAACGAGAACGGCACCATCACCG | |
| | ACGCCGTGGACTGCGCCCTGGACCCCCTGAGCGAGACCAAGTGCACC | |
| | CTGAAGAGCTTCACCGTGGAGAAGGGCATCTACCAGACCAGCAACTTC | |
| | AGGGTGCAGCCCACCGAGAGCATCGTGAGGTTCCCCAACATCACCAA | |
| | CCTGTGCCCCTTCGACGAGGTGTTCAACGCCACCAGGTTCGCCAGCG | |
| | TGTACGCCTGGAACAGGAAGAGGATCAGCAACTGCGTGGCCGACTAC | |
| | AGCGTGCTGTACAACTTCGCCCCCTTCTTCGCCTTCAAGTGCTACGGC | |
| | GTGAGCCCCACCAAGCTGAACGACCTGTGCTTCACCAACGTGTACGC | |
| | CGACAGCTTCGTGATCAGGGGCAACGAGGTGAGCCAGATCGCCCCCG | |
| | GCCAGACCGGCAACATCGCCGACTACAACTACAAGCTGCCCGACGAC | |
| | TTCACCGGCTGCGTGATCGCCTGGAACAGCAACAAGCTGGACAGCAA | |
| | GGTGGGCGGCAACTACAACTACCTGTACAGGCTGTTCAGGAAGAGCA | |
| | ACCTGAAGCCCTTCGAGAGGGACATCAGCACCGAGATCTACCAGGCC | |
| | GGCAACAAGCCCTGCAACGGCGTGGCCGGCTTCAACTGCTACTTCCC | |
| | CCTGAGGAGCTACGGCTTCAGGCCCACCTACGGCGTGGGCCACCAGC | |
| | CCTACAGGGTGGTGGTGCTGAGCTTCGAGCTGCTGCACGCCCCCGCC | |
| | ACCGTGTGCGGCCCCAAGAAGAGCACCAACCTGGTGAAGAACAAGTG | |
| | CGTGAACTTCAACTTCAACGGCCTGACCGGCACCGGCGTGCTGACCG | |
| | AGAGCAACAAGAAGTTCCTGCCCTTCCAGCAGTTCGGCAGGGACATC | |
| | GCCGACACCACCGACGCCGTGAGGGACCCCCAGACCCTGGAGATCCT | |
| | GGACATCACCCCCTGCAGCTTCGGCGGCGTGAGCGTGATCACCCCCG | |
| | GCACCAACACCAGCAACCAGGTGGCCGTGCTGTACCAGGGCGTGAAC | |
| | TGCACCGAGGTGCCCGTGGCCATCCACGCCGACCAGCTGACCCCCAC | |
| | CTGGAGGGTGTACAGCACCGGCAGCAACGTGTTCCAGACCAGGGCCG | |
| | GCTGCCTGATCGGCGCCGAGTACGTGAACAACAGCTACGAGTGCGAC | |
| | ATCCCCATCGGCGCCGGCATCTGCGCCAGCTACCAGACCCAGACCAA | |
| | GAGCCACCAGCAGGCCCAGAGCGTGGCCAGCCAGAGCATCATCGCCT | |
| | ACACCATGAGCCTGGGCGCCGAGAACAGCGTGGCCTACAGCAACAAC | |
| | AGCATCGCCATCCCCACCAACTTCACCATCAGCGTGACCACCGAGATC | |
| | CTGCCCGTGAGCATGACCAAGACCAGCGTGGACTGCACCATGTACAT | |
| | CTGCGGCGACAGCACCGAGTGCAGCAACCTGCTGCTGCAGTACGGCA | |
| | GCTTCTGCACCCAGCTGAAGAGGGCCCTGACCGGCATCGCCGTGGAG | |
| | CAGGACAAGAACACCCAGGAGGTGTTCGCCCAGGTGAAGCAGATCTA | |
| | CAAGACCCCCCCCATCAAGTACTTCGGCGGCTTCAACTTCAGCCAGAT | |
| | CCTGCCCGACCCCAGCAAGCCCAGCAAGAGGAGCCCCATCGAGGAC | |
| | CTGCTGTTCAACAAGGTGACCCTGGCCGACGCCGGCTTCATCAAGCA | |
| | GTACGGCGACTGCCTGGGCGACATCGCCGCCAGGGACCTGATCTGCG | |
| | CCCAGAAGTTCAACGGCCTGACCGTGCTGCCCCCCCTGCTGACCGAC | |
| | GAGATGATCGCCCAGTACACCAGCGCCCTGCTGGCCGGCACCATCAC | |
| | CAGCGGCTGGACCTTCGGCGCCGGCCCCGCCCTGCAGATCCCCTTCC | |
| | CCATGCAGATGGCCTACAGGTTCAACGGCATCGGCGTGACCCAGAAC | |
| | GTGCTGTACGAGAACCAGAAGCTGATCGCCAACCAGTTCAACAGCGC | |
| | CATCGGCAAGATCCAGGACAGCCTGAGCAGCACCCCCAGCGCCCTGG | |
| | GCAAGCTGCAGGACGTGGTGAACCACAACGCCCAGGCCCTGAACACC | |
| | CTGGTGAAGCAGCTGAGCAGCAAGTTCGGCGCCATCAGCAGCGTGCT | |
| | GAACGACATCCTGAGCAGGCTGGACCCCCCCGAGGCCGAGGTGCAG | |
| | ATCGACAGGCTGATCACCGGCAGGCTGCAGAGCCTGCAGACCTACGT | |
| | GACCCAGCAGCTGATCAGGGCCGCCGAGATCAGGGCCAGCGCCAAC | |
| | CTGGCCGCCACCAAGATGAGCGAGTGCGTGCTGGGCCAGAGCAAGA | |
| | GGGTGGACTTCTGCGGCAAGGGCTACCACCTGATGAGCTTCCCCCAG | |
| | AGCGCCCCCCACGGCGTGGTGTTCCTGCACGTGACCTACGTGCCCGC | |
| | CCAGGAGAAGAACTTCACCACCGCCCCCGCCATCTGCCACGACGGCA | |
| | AGGCCCACTTCCCCAGGGAGGGCGTGTTCGTGAGCAACGGCACCCAC | |
| | TGGTTCGTGACCCAGAGGAACTTCTACGAGCCCCAGATCATCACCACC | |
| | GACAACACCTTCGTGAGCGGCAACTGCGACGTGGTGATCGGCATCGT | |
| | GAACAACACCGTGTACGACCCCCTGCAGCCCGAGCTGGACAGCTTCA | |
| | AGGAGGAGCTGGACAAGTACTTCAAGAACCACACCAGCCCCGACGTG | |
| | GACCTGGGCGACATCAGCGGCATCAACGCCAGCGTGGTGAACATCCA | |
| | GAAGGAGATCGACAGGCTGAACGAGGTGGCCAAGAACCTGAACGAGA | |
| | GCCTGATCGACCTGCAGGAGCTGGGCAAGTACGAGCAGTACATCAAG | |
| | TGGCCCTGGTACATCTGGCTGGGCTTCATCGCCGGCCTGATCGCCAT | |
| | CGTGATGGTGACCATCATGCTGTGCTGCATGACCAGCTGCTGCAGCTG | |
| | CCTGAAGGGCTGCTGCAGCTGCGGCAGCTGCTGCAAGTTCGACGAGG | |
| | ACGACAGCGAGCCCGTGCTGAAGGGCGTGAAGCTGCACTACACC | |
| Spike protein | AACCTGATCACCAGGACCCAGAGCTACACCAACAGCTTCACCAGGGG | 6 |
| (Omicron BA.2) | CGTGTACTACCCCGACAAGGTGTTCAGGAGCAGCGTGCTGCACAGCA | |
| with 2 proline | CCCAGGACCTGTTCCTGCCCTTCTTCAGCAACGTGACCTGGTTCCACG | |
| substitution | CCATCCACGTGAGCGGCACCAACGGCACCAAGAGGTTCGACAACCCC | |
| | GTGCTGCCCTTCAACGACGGCGTGTACTTCGCCAGCACCGAGAAGAG | |
| | CAACATCATCAGGGGCTGGATCTTCGGCACCACCCTGGACAGCAAGA | |
| | CCCAGAGCCTGCTGATCGTGAACAACGCCACCAACGTGGTGATCAAG | |
| | GTGTGCGAGTTCCAGTTCTGCAACGACCCCTTCCTGGACGTGTACTAC | |
| | CACAAGAACAACAAGAGCTGGATGGAGAGCGAGTTCAGGGTGTACAG | |
| | CAGCGCCAACAACTGCACCTTCGAGTACGTGAGCCAGCCCTTCCTGAT | |
| | GGACCTGGAGGGCAAGCAGGGCAACTTCAAGAACCTGAGGGAGTTCG | |
| | TGTTCAAGAACATCGACGGCTACTTCAAGATCTACAGCAAGCACACCC | |
| | CCATCAACCTGGGCAGGGACCTGCCCCAGGGCTTCAGCGCCCTGGAG | |
| | CCCCTGGTGGACCTGCCCATCGGCATCAACATCACCAGGTTCCAGAC | |
| | CCTGCTGGCCCTGCACAGGAGCTACCTGACCCCCGGCGACAGCAGCA | |
| | GCGGCTGGACCGCCGGCGCCGCCGCCTACTACGTGGGCTACCTGCA | |
| | GCCCAGGACCTTCCTGCTGAAGTACAACGAGAACGGCACCATCACCG | |
| | ACGCCGTGGACTGCGCCCTGGACCCCCTGAGCGAGACCAAGTGCACC | |
| | CTGAAGAGCTTCACCGTGGAGAAGGGCATCTACCAGACCAGCAACTTC | |
| | AGGGTGCAGCCCACCGAGAGCATCGTGAGGTTCCCCAACATCACCAA | |
| | CCTGTGCCCCTTCGACGAGGTGTTCAACGCCACCAGGTTCGCCAGCG | |
| | TGTACGCCTGGAACAGGAAGAGGATCAGCAACTGCGTGGCCGACTAC | |
| | AGCGTGCTGTACAACTTCGCCCCCTTCTTCGCCTTCAAGTGCTACGGC | |
| | GTGAGCCCCACCAAGCTGAACGACCTGTGCTTCACCAACGTGTACGC | |
| | CGACAGCTTCGTGATCAGGGGCAACGAGGTGAGCCAGATCGCCCCCG | |
| | GCCAGACCGGCAACATCGCCGACTACAACTACAAGCTGCCCGACGAC | |
| | TTCACCGGCTGCGTGATCGCCTGGAACAGCAACAAGCTGGACAGCAA | |
| | GGTGGGCGGCAACTACAACTACCTGTACAGGCTGTTCAGGAAGAGCA | |
| | ACCTGAAGCCCTTCGAGAGGGACATCAGCACCGAGATCTACCAGGCC | |
| | GGCAACAAGCCCTGCAACGGCGTGGCCGGCTTCAACTGCTACTTCCC | |
| | CCTGAGGAGCTACGGCTTCAGGCCCACCTACGGCGTGGGCCACCAGC | |
| | CCTACAGGGTGGTGGTGCTGAGCTTCGAGCTGCTGCACGCCCCCGCC | |
| | ACCGTGTGCGGCCCCAAGAAGAGCACCAACCTGGTGAAGAACAAGTG | |
| | CGTGAACTTCAACTTCAACGGCCTGACCGGCACCGGCGTGCTGACCG | |
| | AGAGCAACAAGAAGTTCCTGCCCTTCCAGCAGTTCGGCAGGGACATC | |
| | GCCGACACCACCGACGCCGTGAGGGACCCCCAGACCCTGGAGATCCT | |
| | GGACATCACCCCCTGCAGCTTCGGCGGCGTGAGCGTGATCACCCCCG | |
| | GCACCAACACCAGCAACCAGGTGGCCGTGCTGTACCAGGGCGTGAAC | |
| | TGCACCGAGGTGCCCGTGGCCATCCACGCCGACCAGCTGACCCCCAC | |
| | CTGGAGGGTGTACAGCACCGGCAGCAACGTGTTCCAGACCAGGGCCG | |
| | GCTGCCTGATCGGCGCCGAGTACGTGAACAACAGCTACGAGTGCGAC | |
| | ATCCCCATCGGCGCCGGCATCTGCGCCAGCTACCAGACCCAGACCAA | |
| | GAGCCACCAGCAGGCCCAGAGCGTGGCCAGCCAGAGCATCATCGCCT | |
| | ACACCATGAGCCTGGGCGCCGAGAACAGCGTGGCCTACAGCAACAAC | |
| | AGCATCGCCATCCCCACCAACTTCACCATCAGCGTGACCACCGAGATC | |
| | CTGCCCGTGAGCATGACCAAGACCAGCGTGGACTGCACCATGTACAT | |
| | CTGCGGCGACAGCACCGAGTGCAGCAACCTGCTGCTGCAGTACGGCA | |
| | GCTTCTGCACCCAGCTGAAGAGGGCCCTGACCGGCATCGCCGTGGAG | |
| | CAGGACAAGAACACCCAGGAGGTGTTCGCCCAGGTGAAGCAGATCTA | |
| | CAAGACCCCCCCCATCAAGTACTTCGGCGGCTTCAACTTCAGCCAGAT | |
| | CCTGCCCGACCCCAGCAAGCCCAGCAAGAGGAGCTTCATCGAGGACC | |
| | TGCTGTTCAACAAGGTGACCCTGGCCGACGCCGGCTTCATCAAGCAGT | |
| | ACGGCGACTGCCTGGGCGACATCGCCGCCAGGGACCTGATCTGCGC | |
| | CCAGAAGTTCAACGGCCTGACCGTGCTGCCCCCCCTGCTGACCGACG | |
| | AGATGATCGCCCAGTACACCAGCGCCCTGCTGGCCGGCACCATCACC | |
| | AGCGGCTGGACCTTCGGCGCCGGCGCCGCCCTGCAGATCCCCTTCG | |
| | CCATGCAGATGGCCTACAGGTTCAACGGCATCGGCGTGACCCAGAAC | |
| | GTGCTGTACGAGAACCAGAAGCTGATCGCCAACCAGTTCAACAGCGC | |
| | CATCGGCAAGATCCAGGACAGCCTGAGCAGCACCGCCAGCGCCCTGG | |
| | GCAAGCTGCAGGACGTGGTGAACCACAACGCCCAGGCCCTGAACACC | |
| | CTGGTGAAGCAGCTGAGCAGCAAGTTCGGCGCCATCAGCAGCGTGCT | |
| | GAACGACATCCTGAGCAGGCTGGACCCCCCCGAGGCCGAGGTGCAG | |
| | ATCGACAGGCTGATCACCGGCAGGCTGCAGAGCCTGCAGACCTACGT | |
| | GACCCAGCAGCTGATCAGGGCCGCCGAGATCAGGGCCAGCGCCAAC | |
| | CTGGCCGCCACCAAGATGAGCGAGTGCGTGCTGGGCCAGAGCAAGA | |
| | GGGTGGACTTCTGCGGCAAGGGCTACCACCTGATGAGCTTCCCCCAG | |
| | AGCGCCCCCCACGGCGTGGTGTTCCTGCACGTGACCTACGTGCCCGC | |
| | CCAGGAGAAGAACTTCACCACCGCCCCCGCCATCTGCCACGACGGCA | |
| | AGGCCCACTTCCCCAGGGAGGGCGTGTTCGTGAGCAACGGCACCCAC | |
| | TGGTTCGTGACCCAGAGGAACTTCTACGAGCCCCAGATCATCACCACC | |
| | GACAACACCTTCGTGAGCGGCAACTGCGACGTGGTGATCGGCATCGT | |
| | GAACAACACCGTGTACGACCCCCTGCAGCCCGAGCTGGACAGCTTCA | |
| | AGGAGGAGCTGGACAAGTACTTCAAGAACCACACCAGCCCCGACGTG | |
| | GACCTGGGCGACATCAGCGGCATCAACGCCAGCGTGGTGAACATCCA | |
| | GAAGGAGATCGACAGGCTGAACGAGGTGGCCAAGAACCTGAACGAGA | |
| | GCCTGATCGACCTGCAGGAGCTGGGCAAGTACGAGCAGTACATCAAG | |
| | TGGCCCTGGTACATCTGGCTGGGCTTCATCGCCGGCCTGATCGCCAT | |
| | CGTGATGGTGACCATCATGCTGTGCTGCATGACCAGCTGCTGCAGCTG | |
| | CCTGAAGGGCTGCTGCAGCTGCGGCAGCTGCTGCAAGTTCGACGAGG | |
| | ACGACAGCGAGCCCGTGCTGAAGGGCGTGAAGCTGCACTACACC | |
| spike | TACCAAGCTACTAGAGTAGTGGTACTTTCATTTGAGCTTCTAAATGCAC | 7 |
| glycoprotein | CTGCCACAGTGTGTGGACCAAAATTGTCCACATCACTAATTAAGAACCA | |
| | GTGTGTCAATTTTAATTTCAATGGACTCAAGGGTACTGGTGTGTTGACT | |
| | GACTCGTCCAAAAAGTTTCAGTCTTTTCAACAATTTGGAAGGGATGCAT | |
| | CTGATTTTACTGACTCAGTACGCGACCCTCAGACACTTCAAATACTTGA | |
| | CATTTCACCATGTTCATTTGGTGGTGTGAGTGTAATAACACCAGGAACA | |
| | AATGCTTCATCTGAAGTAGCCGTTCTATACCAAGATGTAAACTGCACTG | |
| | ATGTTCCCACGGCCATACGTGCTGACCAACTCACACCTGCTTGGCGTG | |
| | TTTACTCTGCTGGAGTAAATGTGTTTCAAACTCAGGCTGGCTGTTTAAT | |
| | AGGAGCGGAACATGTCAATGCTTCATATGAGTGTGACATTCCCATTGG | |
| | TGCAGGCATTTGTGCTAGTTACCATACAGCTTCCCTTTTACGTAATACA | |
| | GGCCAGAAATCAATTGTGGCCTATACTATGTCACTTGGTGCTGAAAACT | |
| | CAATTGCTTATGCTAATAACTCAATTGCCATACCTACAAATTTTTCAATC | |
| | AGTGTCACAACTGAAGTGATGCCTGTTTCAATGGCTAAGACATCAGTA | |
| | GATTGTACAATGTACATCTGTGGTGACTCTCAGGAGTGCAGCAACTTA | |
| | CTACTTCAGTATGGTAGCTTTTGCACACAATTAAATCGTGCCCTTTCAG | |
| | GCATTGCTGTTGAACAGGACAAAAACACTCAAGAGGTTTTTGCCCAAG | |
| | TTAAACAAATGTATAAGACACCAGCCATAAAAGATTTTGGTGGCTTTAA | |
| | TTTCTCACAAATATTGCCTGACCCTTCTAAGCCAACAAAAAGATCATTTA | |
| | TTGAGGATTTACTCTTCAACAAAGTGACTCTCGCTGATGCTGGCTTTAT | |
| | GAAGCAATACGGCGAATGCCTAGGCGATATTAGTGCTAGAGATCTCAT | |
| | TTGTGCGCAGAAGTTCAATGGACTCACTGTCCTTCCACCTCTACTCAC | |
| | GGATGAAATGATTGCTGCTTACACCGCCGCTCTTGTCAGCGGTACTGC | |
| | TACTGCTGGTTGGACATTTGGTGCAGGTGCTGCTCTACAAATACCTTTT | |
| | GCTATGCAAATGGCTTATAGGTTCAATGGCATTGGAGTTACTCAAAATG | |
| | TTCTCTATGAGAACCAGAAGCAGATCGCTAACCAATTTAACAAGGCGAT | |
| | CAGTCAAATTCAAGAATCACTTACTACTACTTCAACTGCATTGGGCAAG | |
| | CTGCAAGACGTCGTCAACCAGAATGCTCAAGCATTGAACACACTTGTT | |
| | AAACAACTAAGTTCTAACTTTGGTGCAATTTCAAGTGTTTTAAATGACAT | |
| | TCTGTCTCGACTYGACAAAGTTGAGGCTGAAGTGCAAATTGATAGGTT | |
| | GATTACTGGCAGATTACAAAGCCTTCAGACCTATGTAACACAACAACTA | |
| | ATCAGAGCTGCTGAAATCAGAGCTTCTGCCAATCTTGCTGCCACTAAG | |
| | ATGTCCGAGTGTGTTCTTGGACAATCAAAAAGAGTTGACTTTTGTGGAA | |
| | AAGGCTATCATCTTATGTCTTTCCCTCAAGCAGCCCCACATGGTGTCGT | |
| | CTTCTTACATGTCACATACGTGCCATCGCAAGAAAGAAACTTCACCACT | |
| | GCCCCAGCAATCTGCCATCAAGGCAAGGCACACTTCCCTCGTGAAGGT | |
| | GTTTTTGTATCTAATGGCACTTCTTGGTTTATCACACAGAGGAACTTCTT | |
| | TTCACCACAAATAATTACAACAGACAATACATTTGTCTCTGGAAATTGTG | |
| | ATGTCGTTATTGGCATCATCAACAATACTGTTTATGATCCTCTGCAACC | |
| | TGAGCTTGACTCATTTAAAGAAGAGCTGGACAAGTACTTCAAAAACCAC | |
| | ACGTCACCTGATGTRGATCTTGGCGACATCTCAGGCATTAATGCTTCA | |
| | GTCGTCAATATTCAAAAAGAAATTGACCGCCTCAATGAGGTTGCCAAAA | |
| | ATCTAAATGAATCGCTCATCGATCTTCAAGAACTTGGAAAATATGAGCA | |

In some embodiments, the Spike protein is encoded by a sequence that is 100%, 98%, 95%, 90%, 85%, 80%, 75%, or 50% identical to any of the aforementioned sequences within Table 1.

Table 2 shows non-limiting examples of proteins that may be used to create a vaccine composition described herein. In some embodiments, the proteins listed below may be arranged in a plurality of combinations. In some embodiments, the proteins may be directly linked together. In other embodiments, the proteins are linked together via a linker.

| Protein | Sequence | SEQ ID NO: |
|---|---|---|
| NSP2 | GCCTACACCAGGTACGTGGACAACAACTTCTGCGGCCCCGACGGCTA | 8 |
| | CCCCCTGGAGTGCATCAAGGACCTGCTGGCCAGGGCCGGCAAGGCC | |
| | AGCTGCACCCTGAGCGAGCAGCTGGACTTCATCGACACCAAGAGGGG | |
| | CGTGTACTGCTGCAGGGAGCACGAGCACGAGATCGCCTGGTACACCG | |
| | AGAGGAGCGAGAAGAGCTACGAGCTGCAGACCCCCTTCGAGATCAAG | |
| | CTGGCCAAGAAGTTCGACACCTTCAACGGCGAGTGCCCCAACTTCGTG | |
| | TTCCCCCTGAACAGCATCATCAAGACCATCCAGCCCAGGGTGGAGAAG | |
| | AAGAAGCTGGACGGCTTCATGGGCAGGATCAGGAGCGTGTACCCCGT | |
| | GGCCAGCCCCAACGAGTGCAACCAGATGTGCCTGAGCACCCTGATGA | |
| | AGTGCGACCACTGCGGCGAGACCAGCTGGCAGACCGGCGACTTCGTG | |
| | AAGGCCACCTGCGAGTTCTGCGGCACCGAGAACCTGACCAAGGAGGG | |
| | CGCCACCACCTGCGGCTACCTGCCCCAGAACGCCGTGGTGAAGATCT | |
| | ACTGCCCCGCCTGCCACAACAGCGAGGTGGGCCCCGAGCACAGCCT | |
| | GGCCGAGTACCACAACGAGAGCGGCCTGAAGACCATCCTGAGGAAGG | |
| | GCGGCAGGACCATCGCCTTCGGCGGCTGCGTGTTCAGCTACGTGGGC | |
| | TGCCACAACAAGTGCGCCTACTGGGTGCCCAGGGCCAGCGCCAACAT | |
| | CGGCTGCAACCACACCGGCGTGGTGGGCGAGGGCAGCGAGGGCCTG | |
| | AACGACAACCTGCTGGAGATCCTGCAGAAGGAGAAGGTGAACATCAA | |
| | CATCGTGGGCGACTTCAAGCTGAACGAGGAGATCGCCATCATCCTGG | |
| | CCAGCTTCAGCGCCAGCACCAGCGCCTTCGTGGAGACCGTGAAGGGC | |
| | CTGGACTACAAGGCCTTCAAGCAGATCGTGGAGAGCTGCGGCAACTT | |
| | CAAGGTGACCAAGGGCAAGGCCAAGAAGGGCGCCTGGAACATCGGC | |
| | GAGCAGAAGAGCATCCTGAGCCCCCTGTACGCCTTCGCCAGCGAGGC | |
| | CGCCAGGGTGGTGAGGAGCATCTTCAGCAGGACCCTGGAGACCGCCC | |
| | AGAACAGCGTGAGGGTGCTGCAGAAGGCCGCCATCACCATCCTGGAC | |
| | GGCATCAGCCAGTACAGCCTGAGGCTGATCGACGCCATGATGTTCAC | |
| | CAGCGACCTGGCCACCAACAACCTGGTGGTGATGGCCTACATCACCG | |
| | GCGGCGTGGTGCAGCTGACCAGCCAGTGGCTGACCAACATCTTCGGC | |
| | ACCGTGTACGAGAAGCTGAAGCCCGTGCTGGACTGGCTGGAGGAGAA | |
| | GTTCAAGGAGGGCGTGGAGTTCCTGAGGGACGGCTGGGAGATCGTGA | |
| | AGTTCATCAGCACCTGCGCCTGCGAGATCGTGGGGGGCCAGATCGTG | |
| | ACCTGCGCCAAGGAGATCAAGGAGAGCGTGCAGACCTTCTTCAAGCT | |
| | GGTGAACAAGTTCCTGGCCCTGTGCGCCGACAGCATCATCATCGGCG | |
| | GCGCCAAGCTGAAGGCCCTGAACCTGGGCGAGACCTTCGTGACCCAC | |
| | AGCAAGGGCCTGTACAGGAAGTGCGTGAAGAGCAGGGAGGAGACCG | |
| | GCCTGCTGATGCCCCTGAAGGCCCCCAAGGAGATCATCTTCCTGGAG | |
| | GGCGAGACCCTGCCCACCGAGGTGCTGACCGAGGAGGTGGTGCTGA | |
| | AGACCGGCGACCTGCAGCCCCTGGAGCAGCCCACCAGCGAGGCCGT | |
| | GGAGGCCCCCCTGGTGGGCACCCCCGTGTGCATCAACGGCCTGATGC | |
| | TGCTGGAGATCAAGGACACCGAGAAGTACTGCGCCCTGGCCCCCAAC | |
| | ATGATGGTGACCAACAACACCTTCACCCTGAAGGGCGGC | |
| NSP3 | GCCCCCACCAAGGTGACCTTCGGCGACGACACCGTGATCGAGGTGCA | 9 |
| | GGGCTACAAGAGCGTGAACATCACCTTCGAGCTGGACGAGAGGATCG | |
| | ACAAGGTGCTGAACGAGAAGTGCAGCGCCTACACCGTGGAGCTGGGC | |
| | ACCGAGGTGAACGAGTTCGCCTGCGTGGTGGCCGACGCCGTGATCAA | |
| | GACCCTGCAGCCCGTGAGCGAGCTGCTGACCCCCCTGGGCATCGACC | |
| | TGGACGAGTGGAGCATGGCCACCTACTACCTGTTCGACGAGAGCGGC | |
| | GAGTTCAAGCTGGCCAGCCACATGTACTGCAGCTTCTACCCCCCCGAC | |
| | GAGGACGAGGAGGAGGGCGACTGCGAGGAGGAGGAGTTCGAGCCCA | |
| | GCACCCAGTACGAGTACGGCACCGAGGACGACTACCAGGGCAAGCCC | |
| | CTGGAGTTCGGCGCCACCAGCGCCGCCCTGCAGCCCGAGGAGGAGC | |
| | AGGAGGAGGACTGGCTGGACGACGACAGCCAGCAGACCGTGGGCCA | |
| | GCAGGACGGCAGCGAGGACAACCAGACCACCACCATCCAGACCATCG | |
| | TGGAGGTGCAGCCCCAGCTGGAGATGGAGCTGACCCCCGTGGTGCA | |
| | GACCATCGAGGTGAACAGCTTCAGCGGCTACCTGAAGCTGACCGACA | |
| | ACGTGTACATCAAGAACGCCGACATCGTGGAGGAGGCCAAGAAGGTG | |
| | AAGCCCACCGTGGTGGTGAACGCCGCCAACGTGTACCTGAAGCACGG | |
| | CGGCGGCGTGGCCGGCGCCCTGAACAAGGCCACCAACAACGCCATG | |
| | CAGGTGGAGAGCGACGACTACATCGCCACCAACGGCCCCCTGAAGGT | |
| | GGGCGGCAGCTGCGTGCTGAGCGGCCACAACCTGGCCAAGCACTGC | |
| | CTGCACGTGGTGGGCCCCAACGTGAACAAGGGCGAGGACATCCAGCT | |
| | GCTGAAGAGCGCCTACGAGAACTTCAACCAGCACGAGGTGCTGCTGG | |
| | CCCCCCTGCTGAGCGCCGGCATCTTCGGCGCCGACCCCATCCACAGC | |
| | CTGAGGGTGTGCGTGGACACCGTGAGGACCAACGTGTACCTGGCCGT | |
| | GTTCGACAAGAACCTGTACGACAAGCTGGTGAGCAGCTTCCTGGAGAT | |
| | GAAGAGCGAGAAGCAGGTGGAGCAGAAGATCGCCGAGATCCCCAAG | |
| | GAGGAGGTGAAGCCCTTCATCACCGAGAGCAAGCCCAGCGTGGAGCA | |
| | GAGGAAGCAGGACGACAAGAAGATCAAGGCCTGCGTGGAGGAGGTG | |
| | ACCACCACCCTGGAGGAGACCAAGTTCCTGACCGAGAACCTGCTGCT | |
| | GTACATCGACATCAACGGCAACCTGCACCCCGACAGCGCCACCCTGG | |
| | TGAGCGACATCGACATCACCTTCCTGAAGAAGGACGCCCCCTACATCG | |
| | TGGGCGACGTGGTGCAGGAGGGCGTGCTGACCGCCGTGGTGATCCC | |
| | CACCAAGAAGGCCGGCGGCACCACCGAGATGCTGGCCAAGGCCCTG | |
| | AGGAAGGTGCCCACCGACAACTACATCACCACCTACCCCGGCCAGGG | |
| | CCTGAACGGCTACACCGTGGAGGAGGCCAAGACCGTGCTGAAGAAGT | |
| | GCAAGAGCGCCTTCTACATCCTGCCCAGCATCATCAGCAACGAGAAGC | |
| | AGGAGATCCTGGGCACCGTGAGCTGGAACCTGAGGGAGATGCTGGCC | |
| | CACGCCGAGGAGACCAGGAAGCTGATGCCCGTGTGCGTGGAGACCAA | |
| | GGCCATCGTGAGCACCATCCAGAGGAAGTACAAGGGCATCAAGATCC | |
| | AGGAGGGCGTGGTGGACTACGGCGCCAGGTTCTACTTCTACACCAGC | |
| | AAGACCACCGTGGCCAGCCTGATCAACACCCTGAACGACCTGAACGA | |
| | GACCCTGGTGACCATGCCCCTGGGCTACGTGACCCACGGCCTGAACC | |
| | TGGAGGAGGCCGCCAGGTACATGAGGAGCCTGAAGGTGCCCGCCAC | |
| | CGTGAGCGTGAGCAGCCCCGACGCCGTGACCGCCTACAACGGCTACC | |
| | TGACCAGCAGCAGCAAGACCCCCGAGGAGCACTTCATCGAGACCATC | |
| | AGCCTGGCCGGCAGCTACAAGGACTGGAGCTACAGCGGCCAGAGCA | |
| | CCCAGCTGGGCATCGAGTTCCTGAAGAGGGGCGACAAGAGCGTGTAC | |
| | TACACCAGCAACCCCACCACCTTCCACCTGGACGGCGAGGTGATCAC | |
| | CTTCGACAACCTGAAGACCCTGCTGAGCCTGAGGGAGGTGAGGACCA | |
| | TCAAGGTGTTCACCACCGTGGACAACATCAACCTGCAC | |
| NSP14 | GCCGAGAACGTGACCGGCCTGTTCAAGGACTGCAGCAAGGTGATCAC | 10 |
| | CGGCCTGCACCCCACCCAGGCCCCCACCCACCTGAGCGTGGACACCA | |
| | AGTTCAAGACCGAGGGCCTGTGCGTGGACATCCCCGGCATCCCCAAG | |
| | GACATGACCTACAGGAGGCTGATCAGCATGATGGGCTTCAAGATGAAC | |
| | TACCAGGTGAACGGCTACCCCAACATGTTCATCACCAGGGAGGAGGC | |
| | CATCAGGCACGTGAGGGCCTGGATCGGCTTCGACGTGGAGGGCTGCC | |
| | ACGCCACCAGGGAGGCCGTGGGCACCAACCTGCCCCTGCAGCTGGG | |
| | CTTCAGCACCGGCGTGAACCTGGTGGCCGTGCCCACCGGCTACGTGG | |
| | ACACCCCCAACAACACCGACTTCAGCAGGGTGAGCGCCAAGCCCCCC | |
| | CCCGGCGACCAGTTCAAGCACCTGATCCCCCTGATGTACAAGGGCCT | |
| | GCCCTGGAACGTGGTGAGGATCAAGATCGTGCAGATGCTGAGCGACA | |
| | CCCTGAAGAACCTGAGCGACAGGGTGGTGTTCGTGCTGTGGGCCCAC | |
| | GGCTTCGAGCTGACCAGCATGAAGTACTTCGTGAAGATCGGCCCCGA | |
| | GAGGACCTGCTGCCTGTGCGACAGGAGGGCCACCTGCTTCAGCACCG | |
| | CCAGCGACACCTACGCCTGCTGGCACCACAGCATCGGCTTCGACTAC | |
| | GTGTACAACCCCTTCATGATCGACGTGCAGCAGTGGGGCTTCACCGG | |
| | CAACCTGCAGAGCAACCACGACCTGTACTGCCAGGTGCACGGCAACG | |
| | CCCACGTGGCCAGCTGCGACGCCATCATGACCAGGTGCCTGGCCGTG | |
| | CACGAGTGCTTCGTGAAGAGGGTGGACTGGACCATCGAGTACCCCAT | |
| | CATCGGCGACGAGCTGAAGATCAACGCCGCCTGCAGGAAGGTGCAGC | |
| | ACATGGTGGTGAAGGCCGCCCTGCTGGCCGACAAGTTCCCCGTGCTG | |
| | CACGACATCGGCAACCCCAAGGCCATCAAGTGCGTGCCCCAGGCCGA | |
| | CGTGGAGTGGAAGTTCTACGACGCCCAGCCCTGCAGCGACAAGGCCT | |
| | ACAAGATCGAGGAGCTGTTCTACAGCTACGCCACCCACAGCGACAAGT | |
| | TCACCGACGGCGTGTGCCTGTTCTGGAACTGCAACGTGGACAGGTAC | |
| | CCCGCCAACAGCATCGTGTGCAGGTTCGACACCAGGGTGCTGAGCAA | |
| | CCTGAACCTGCCCGGCTGCGACGGCGGCAGCCTGTACGTGAACAAGC | |
| | ACGCCTTCCACACCCCCGCCTTCGACAAGAGCGCCTTCGTGAACCTGA | |
| | AGCAGCTGCCCTTCTTCTACTACAGCGACAGCCCCTGCGAGAGCCAC | |
| | GGCAAGCAGGTGGTGAGCGACATCGACTACGTGCCCCTGAAGAGCGC | |
| | CACCTGCATCACCAGGTGCAACCTGGGGGGCGCCGTGTGCAGGCACC | |
| | ACGCCAACGAGTACAGGCTGTACCTGGACGCCTACAACATGATGATCA | |
| | GCGCCGGCTTCAGCCTGTGGGTGTACAAGCAGTTCGACACCTACAAC | |
| | CTGTGGAACACCTTCACCAGGCTGCAG | |
| Nucleocapsid | AGCGACAACGGCCCCCAGAACCAGAGGAACGCCCCCAGGATCACCTT | 11 |
| | CGGCGGCCCCAGCGACAGCACCGGCAGCAACCAGAACGGCGAGAGG | |
| | AGCGGCGCCAGGAGCAAGCAGAGGAGGCCCCAGGGCCTGCCCAACA | |
| | ACACCGCCAGCTGGTTCACCGCCCTGACCCAGCACGGCAAGGAGGAC | |
| | CTGAAGTTCCCCAGGGGCCAGGGCGTGCCCATCAACACCAACAGCAG | |
| | CCCCGACGACCAGATCGGCTACTACAGGAGGGCCACCAGGAGGATCA | |
| | GGGGCGGCGACGGCAAGATGAAGGACCTGAGCCCCAGGTGGTACTT | |
| | CTACTACCTGGGCACCGGCCCCGAGGCCGGCCTGCCCTACGGCGCC | |
| | AACAAGGACGGCATCATCTGGGTGGCCACCGAGGGCGCCCTGAACAC | |
| | CCCCAAGGACCACATCGGCACCAGGAACCCCGCCAACAACGCCGCCA | |
| | TCGTGCTGCAGCTGCCCCAGGGCACCACCCTGCCCAAGGGCTTCTAC | |
| | GCCGAGGGCAGCAGGGGCGGCAGCCAGGCCAGCAGCAGGAGCAGC | |
| | AGCAGGAGCAGGAACAGCAGCAGGAACAGCACCCCCGGCAGCAGCA | |
| | GGGGCACCAGCCCCGCCAGGATGGCCGGCAACGGCGGCGACGCCG | |
| | CCCTGGCCCTGCTGCTGCTGGACAGGCTGAACCAGCTGGAGAGCAAG | |
| | ATGAGCGGCAAGGGCCAGCAGCAGCAGGGCCAGACCGTGACCAAGA | |
| | AGAGCGCCGCCGAGGCCAGCAAGAAGCCCAGGCAGAAGAGGACCGC | |
| | CACCAAGGCCTACAACGTGACCCAGGCCTTCGGCAGGAGGGGCCCC | |
| | GAGCAGACCCAGGGCAACTTCGGCGACCAGGAGCTGATCAGGCAGG | |
| | GCACCGACTACAAGCACTGGCCCCAGATCGCCCAGTTCGCCCCCAGC | |
| | GCCAGCGCCTTCTTCGGCATGAGCAGGATCGGCATGGAGGTGACCCC | |
| | CAGCGGCACCTGGCTGACCTACACCGGCGCCATCAAGCTGGACGACA | |
| | AGGACCCCAACTTCAAGGACCAGGTGATCCTGCTGAACAAGCACATCG | |
| | ACGCCTACAAGACCTTCCCCCCCACCGAGCCCAAGAAGGACAAGAAG | |
| | AAGAAGGCCGACGAGACCCAGGCCCTGCCCCAGAGGCAGAAGAAGC | |
| | AGCAGACCGTGACCCTGCTGCCCGCCGCCGACCTGGACGACTTCAGC | |
| | AAGCAGCTGCAGCAGAGCATGAGCAGCGCCGACAGCACCCAGGCC | |

In some embodiments, the portion of the NSP2 protein is encoded by a sequence that is 100%, 98%, 95%, 90%, 85%, 80%, 75%, or 50% identical to SEQ ID NO: 8. In some embodiments, the portion of the NSP3 protein is encoded by a sequence that is 100%, 98%, 95%, 90%, 85%, 80%, 75%, or 50% identical to SEQ ID NO: 9. In some embodiments, the portion of the NSP14 protein is encoded by a sequence that is 100%, 98%, 95%, 90%, 85%, 80%, 75%, or 50% identical to SEQ ID NO: 10. In some embodiments, the portion of the Nucleoprotein is encoded by a sequence that is 100%, 98%, 95%, 90%, 85%, 80%, 75%, or 50% identical to SEQ ID NO: 11.

### Influenza Antigen/Large Influenza Sequences:

In some embodiments, the portion of an influenza hemagglutinin (HA) protein is highly conserved among human influenza viruses. The portion of an influenza hemagglutinin (HA) protein may be derived from one or more of: the H1N1 virus strain, the H3N2 virus strain, influenza B virus strains, or variants thereof.

In some embodiments, the H1N1 virus strains or variants are selected from 28566 available complete genome sequences in NCBI for the hemagglutinin (HA) gene. Some of the prominent strains are: OK384178.1, OM642156.1, OM654386.1, OL840606.1, OK625377.1, OM865246.1, OM935941.1, OM642158.1, OM935953.1, MW840068.1, MW839847.1, MW839825.1, MW930730.1, MT227010.1, LC638096.1, LC638077.1, LC637999.1, and LC645067.1. In some embodiments, the H3N2 virus strains or variants are selected from 33620 available complete genome sequences in NCBI for the hemagglutinin (HA) gene. Some of the prominent strains are: MZ005227.1, MW849238.1, MZ203409.1, MZ198318.1, MZ198312.1, MZ198295.1, MZ198289.1, MZ198265.1, MW789449.1, MW798370.1, MW790182.1, MW789645.1, MW789778.1, MW789685.1, MW789659.1, and MW790001.1. In some embodiments, the influenza B virus strains or variants are selected from 16596 available complete genome sequences in NCBI for the hemagglutinin (HA) gene. Some of the prominent strains are: M10298.1, MT738525.1, MT808088.1, MT056751.1, MT314641.1, MT874090.1, MT242979.1, MT315665.1, MT1055640.1, MT057563.1, MT056955.1, MT243019.1, MT306916.1, MT057571.1, MT314793.1, MT343026.1, MT874109.1, MT243795.1, MT315769.1, and KX885875.1

Table 3: Shows non-limiting examples of a portion of an influenza hemagglutinin (HA) protein that may be used in accordance with the present invention.

| Influenza | | |
|---|---|---|
| Antigen | Sequence | SEQ ID NO: |
| HA (nucleotide) | TTCGGAGCTATTGCTGGTTTCTTGGAAGGAGGATGGGAAGGAATGAT | 12 |
| | TGCAGGTTGGCACGGATACACATCTCATGGAGCACATGGAGTAGCA | |
| | GTGGCAGCAGACCTTAAGAGTACCCA | |
| HA | FGAIAGFLEGGWEGMIAGWHGYTSHGAHGVAVAADLKSTX | 13 |
| (amino acid) | | |
| HA-H1N1 | ATGAAGGCAATACTAGTAGTTCTGCTATATACATTTGCAACCGCAAAT | 14 |
| | GCAGACACATTATGTATAGGTTATCATGCGAACAACTCAACAGACACT | |
| | GTAGACACAGTACTAGAAAAGAATGTAACAGTAACACACTCTGTTAAC | |
| | CTTCTAGAAGACAAGCATAACGGGAAACTATGCAAACTAAGAGGGGT | |
| | AGCCCCATTGCATTTGGGTAAATGTAACATTGCTGGCTGGATCCTGG | |
| | GAAATCCAGAGTGTGAATCACTCTCCACAGCAAGCTCATGGTCCTAC | |
| | ATTGTGGAAACATCTAGTTCAGACAATGGAACGTGTTACCCAGGAGA | |
| | TTTCATCGATTATGAGGAGCTAAGAGAGCAATTGAGCTCAGTGTCAT | |
| | CATTTGAAAGGTTTGAGATATTCCCCAAGACAAGTTCATGGCCCAATC | |
| | ATGACTCGAACAAAGGTGTAACGACAGCATGTCCTCATGCTGGAGCA | |
| | AAAAGCTTCTACAAAAATTTAATATGGCTAGTTAAAAAAGGAAATTCAT | |
| | ACCCAAAGCTCAGCAAATCCTACATTAATGATAAAGGGAAAGAAGTC | |
| | CTCGTGCTATGGGGCATTCACCATCCACCTACTAGTGCTGACCAACA | |
| | AAGTCTCTATCAGAATGCAGATGCATATGTTTTTGTGGGGACATCAAG | |
| | ATACAGCAAGAAGTTCAAGCCGGAAATAGCAATAAGGCCCAAAGTGA | |
| | GGGATCAAGAAGGGAGAATGAACTATTACTGGACACTAGTAGAGCC | |
| | GGGAGACAAAATAACATTCGAAGCAACTGGAAATCTAGTGGTACCGA | |
| | GATATGCATTCGCAATGGAAAGAAATGCTGGATCTGGTATTATCATTT | |
| | CAGATACACCAGTCCACGATTGCAATACAACTTGTCAGACACCCAAG | |
| | GGTGCTATAAACACCAGCCTCCCATTTCAGAACATACATCCGATCAC | |
| | AATTGGAAAATGTCCAAAATATGTAAAAAGCACAAAATTGAGACTGGC | |
| | CACAGGATTGAGGAATGTCCCGTCCATTCAATCTAGAGGCCTATTTG | |
| | GGGCCATTGCCGGTTTCATTGAAGGGGGGTGGACAGGGATGGTAGA | |
| | TGGATGGTACGGTTATCACCATCAAAATGAGCAGGGGTCAGGATATG | |
| | CAGCCGACCTGAAGAGCACACAGAATGCCATTGACGAGATTACTAAC | |
| | AAAGTAAACTCTGTTATTGAAAAAATGAATACACAGTTCACAGCAGTA | |
| | GGTAAAGAGTTCAACCACCTGGAAAAAAGAATAGAGAATTTAAATAAA | |
| | AAAGTTGATGATGGTTTCCTGGACATTTGGACTTACAATGCCGAACT | |
| | GTTGGTTCTATTGGAAAATGAAAGAACTTTGGACTACCACGATTCAAA | |
| | GGTGAAGAACTTATATGAAAAGGTAAGAAGCCAGTTAAAAAACAATG | |
| | CCAAGGAAATTGGAAACGGCTGCTTTGAATTTTACCACAAATGTGATA | |
| | ACACGTGCATGGAAAGTGTCAAAAATGGGACTTATGACTACCCAAAA | |
| | TACTCAGAGGAAGCAAAATTAAACAGAGAAGAAATAGATGGGGTAAA | |
| | GCTGGAATCAACAAGGATTTACCAGATTTTGGCGATCTATTCAACCGT | |
| | CGCCAGTTCATTGGTACTGGTAGTCTCCCTGGGGGCAATCAGTTTCT | |
| | GGATGTGCTCTAATGGGTCTCTACAGTGTAGAATATGTATTTAA | |
| HA H3N2 | AGCAAAAGCAGGGGATAATTCTATTAACCATGAAGACTATCATTGCTT | 15 |
| | TGAGCTACATTCTATGTCTGGTTTTCGCTCAAAAACTTCCTGGAAATG | |
| | ACAATAGCACTGCAACGCTGTGCCTTGGGCACCATGCAGTACCAAAC | |
| | GGAACGATAGTGAAAACAATCACGAATGACCAAATTGAAGTTACTAAT | |
| | GCTACTGAGCTGGTTCAGAATTCCTCAATAGGTGAAATATGCGACAG | |
| | TCCTCATCAGATCCTTGATGGAGAAAACTGCACACTAATAGATGCTCT | |
| | ATTGGGAGACCCTCAGTGTGATGGCTTTCAAAATAAGAAATGGGACC | |
| | TTTTTGTTGAACGAAGCAAAGCCTACAGCAACTGTTACCCTTATGATG | |
| | TGCCGGATTATGCCTCCCTTAGGTCACTAGTTGCCTCATCCGGCACA | |
| | CTGGAGTTTAACAATGAAAGCTTCAATTGGGCTGGAGTCACTCAAAA | |
| | CGGAACAAGTTCTGCTTGCATAAGGGGATCTAATAGTAGTTTCTTTAG | |
| | TAGATTAAATTGGTTGACCCACTTAAACTTCAAGTACCCAGCATTGAA | |
| | CGTGACTATGCCAAACAATGAACAATTTGACAAATTGTACATTTGGGG | |
| | GGTTCACCACCCGGGTACGGACAAGGACCAAATCTTCCTGTATGCTC | |
| | AATCATCAGGAAGAATCACAGTATCTACCAAAAGAAGCCAACAAGCT | |
| | GTAATCCCGAATATCGGATCTAGACCCAGAATAAGGAATATCCCTAG | |
| | CAGAATAAGCATCTATTGGACAATAGTAAAACCGGGAGACATACTTTT | |
| | GATTAACAGCACAGGGAATCTAATTGCTCCTAGGGGTTACTTCAAAAT | |
| | ACGAAGTGGGAAAAGCTCAATAATGAGATCAGATGCACCCATTGGCA | |
| | AATGCAAGTCTGAATGCATCACTCCAAATGGAAGCATTCCCAATGAC | |
| | AAACCATTCCAAAATGTAAACAGGATCACATACGGGGCCTGTCCCAG | |
| | ATATGTTAAGCAAAGCACTCTGAAATTGGCAACAGGAATGCGAAATG | |
| | TACCAGAGAAACAAACTAGAGGCATATTTGGCGCAATAGCGGGTTTC | |
| | ATAGAAAATGGTTGGGAGGGAATGGTGGATGGTTGGTACGGTTTCA | |
| | GGCATCAAAATTCTGAGGGAAGAGGACAAGCAGCAGATCTCAAAAG | |
| | CACTCAAGCAGCAATCGATCAAATCAATGGGAAGCTGAATCGATTGA | |
| | TCGGGAAAACCAACGAGAAATTCCATCAGATTGAGAAAGAATTCTCA | |
| | GAAGTAGAAGGGAGAATTCAGGACCTTGAGAAATATGTTGAGGACAC | |
| | AAAAATAGATCTCTGGTCATACAACGCAGAGCTTCTTGTTGCCCTGG | |
| | AAAACCAACATACAATTGATCTAACTGACTCAGAAATGAACAAACTGT | |
| | TTGAAAAAACAAAGAAGCAACTGAGGGAAAATGCTGAGGATATGGGC | |
| | AATGGTTGTTTCAAAATATACCACAAATGTGACAATGCCTGCATAGGA | |
| | TCAATCAGAAATGGAACTTATGACCACGATGTATACAGGGATGAAGC | |
| | ATTAAACAACCGGTTCCAGATCAAGGGAGTTGAGCTGAAGTCAGGGT | |
| | ACAAAGATTGGATCCTATGGATTTCCTTTGCCATATCATGTTTTTTGCT | |
| | TTGTGTTGCTTTGTTGGGGTTCATCATGTGGGCCTGCCAAAAGGGCA | |
| | ACATTAGGTGCAACATTTGCATTTGAGTGCATTAATTAAAAACACCCT | |
| | TGTTTCTACT | |
| HA Influenza B | ATTTTCTAATATCCACAAAATGAAGGCAATAATTGTACTACTCATGGTA | 16 |
| | GTAACATCCAATGCAGATCGAATCTGCACTGGGATAACATCGTCAAA | |
| | CTCACCACATGTCGTCAAAACTGCTACTCAAGGGGAGGTCAACGTGA | |
| | CCGGTGTAATACCACTGACAACAACACCCACCAAATCTCATTTTGCAA | |
| | ATCTCAAAGGAACAGAAACCAGGGGGAAACTATGCCCAAAATGCCTC | |
| | AACTGCACAGATCTGGATGTAGCCTTGGGCAGACCAAAATGCACAG | |
| | GGAAAATACCCTCTGCAAGGGTTTCAATACTCCATGAAGTCAGACCT | |
| | GTTACATCTGGGTGCTTTCCTATAATGCACGATAGAACAAAAATTAGA | |
| | CAGCTGCCTAACCTTCTCCGAGGATACGAACATGTCAGGTTATCAAC | |
| | TCACAACGTTATCAATGCAAAAGATGCACCAGGAAGACCCTACGAAA | |
| | TTGGAACCTCAGGGTCTTGCCCTAACATTACCAATGGAAACGGATTC | |
| | TTCGCAACAATGGCTTGGGCCGTCCCAAAAAACAAAACAGCAACAAA | |
| | TCCATTAACAATAGAAGTACCATACATTTGTACAGAAGGAGAAGACCA | |
| | AATTACCGTTTGGGGGTTCCACTCTGACAACGAGACCCAAATGGCAA | |
| | AGCTCTATGGGGACTCAAAGCCCCAGAAGTTCACCTCATCTGCCAAC | |
| | GGAGTGACCACACATTACGTTTCACAGATTGGTGGCTTCCCAAATCA | |
| | AACAGAAGACGGAGGACTACCACAAAGTGGCAGAATTGTTGTTGATT | |
| | ACATGGTGCAGAAATCTGGAAAAACAGGAACAATTACCTATCAAAGA | |
| | GGTATTTTATTGCCTCAAAAAGTGTGGTGCGCAAGTGGCAGGAGCAA | |
| | GGTAATAAAAGGATCCTTGCCCTTAATTGGAGAAGCAGATTGCCTCC | |
| | ATGAAAAATACGGTGGATTAAACAAAAGCAAGCCTTACTACACAGGG | |
| | GAACATGCAAAGGCCATAGGAAATTGCCCAATATGGGTGAAAACACC | |
| | CTTGAAGCTGGCCAATGGAACCAAATATAGACCCCCTGCAAAACTAT | |
| | TAAAGGAAAGAGGTTTCTTCGGAGCCATTGCTGGTTTCTTAGAGGGA | |
| | GGATGGGAAGGAATGATTGCAGGTTGGCACGGATACACATCCCATG | |
| | GGGCACATGGAGTAGCGGTGGCAGCTGACCTTAAGAGCACTCAAGA | |
| | GGCCATAAACAAGATAACAAAAAATCTCAACTCTTTGAGTGAGCTGG | |
| | AAGTAAAGAATCTTCAAAGACTAAGCGGTGCCATGGATGAACTCCAC | |
| | AACGAAATACTAGAACTAGATGAGAAAGTGGATGATCTCAGAGCTGA | |
| | CACAATAAGCTCACAAATAGAACTCGCAGTCCTGCTTTCCAATGAAG | |
| | GAATAATAAACAGTGAAGATGAACATCTCTTGGCGCTTGAAAGAAAG | |
| | CTGAAGAAAATGCTGGGCCCCTCTGCTGTAGAGATAGGGAATGGAT | |
| | GCTTTGAAACCAAACACAAGTGCAACCAGACCTGCCTCGACAGAATA | |
| | GCTGCTGGTACCTTTGATGCAGGAGAATTTTCTCTCCCCACCTTTGAT | |
| | TCACTGAATATTACTGCTGCATCTTTAAATGACGACGGATTGGACAAT | |
| | CATACTATACTGCTTTACTACTCAACTGCTGCCTCCAGTTTGGCTGTA | |
| | ACACTGATGATAGCTATCTTTGTTGTTTATATGGTCTCCAGAGACAAT | |
| | GTTTCTTGCTCCATTTGTCTATAAGGAAAGTTAAGCCCTGTATTTTCC | |
| | TTTATTGTAGTGCTTGTTTGCTTGTTGTCATTACAAAGAAACGTTATTG | |
| | AAAAAT | |

In some embodiments, the vaccine compositions described herein comprises at least a portion of the aforementioned antigens; e.g., the portion of the antigens may be an immunogenic fragment, e.g., an epitope. In some embodiments, the portion of the HA protein is encoded by a sequence that is 100%, 98%, 95%, 90%, 85%, 80%, 75%, or 50% identical to SEQ ID NO: 12. In some embodiments, the portion of the HA protein comprises a sequence that is 100%, 98%, 95%, 90%, 85%, 80%, 75%, or 50% identical to SEQ ID NO: 13. In some embodiments, the portion of the HA-H1N1 protein is encoded by a sequence that is 100%, 98%, 95%, 90%, 85%, 80%, 75%, or 50% identical to SEQ ID NO: 14. In some embodiments, the portion of the HA H3N2 protein is encoded by a sequence that is 100%, 98%, 95%, 90%, 85%, 80%, 75%, or 50% identical to SEQ ID NO: 15. In some embodiments, the portion of the HA Influenza B protein is encoded by a sequence that is 100%, 98%, 95%, 90%, 85%, 80%, 75%, or 50% identical to SEQ ID NO: 16.

### Vaccine Composition:

In some embodiments, the compositions described herein may be used to prevent a coronavirus and/or influenza infection prophylactically in a subject. In some embodiments, the compositions described herein may elicit an immune response in a subject.

In some embodiments, the vaccine composition comprises one antigen. In some embodiments, the vaccine composition comprises one or more antigens. In some embodiments, the vaccine composition comprises two antigens. In some embodiments, the vaccine composition comprises two or more antigens. In some embodiments, the vaccine composition comprises three antigens. In some embodiments, the vaccine composition comprises three or more antigens. In some embodiments, the vaccine composition comprises four antigens. In some embodiments, the vaccine composition comprises four or more antigens. In some embodiments, the vaccine composition comprises five or more antigens, e.g., 5, 6, 7, 8, etc. In some embodiments, the vaccine composition comprises one antigen.

In some embodiments, the vaccine composition comprises one large sequence. In some embodiments, the vaccine composition comprises one or more large sequences. In some embodiments, the vaccine composition comprises two or more large sequences. In some embodiments, the vaccine composition comprises three or more large sequences. In some embodiments, the vaccine composition comprises four or more large sequences. In some embodiments, the vaccine composition comprises five or more large sequences, e.g., 5, 6, 7, 8, etc.

Table 4, FIG. 2, and FIG. 3B shows additional examples of vaccine compositions described herein (FIG. 3A showIn some embodiments, the portion of the NSP2 protein is encoded by a sequence that is 100%, 98%, 95%, 90%, 85%, 80%, 75%, or 50% identical to SEQ ID NO: 8.s non-limiting examples of coronavirus and influenza virus proteins that may be considered, or may be the proteins from which partial proteins are derived, for vaccine candidates). The present invention is not limited to the examples in Table 4:

TABLE 4

| | | SEQ |
|---|---|---|
| Vaccine | | ID |
| Candidate | Sequence | NO: |
| 1 | TAGTTATTAATAGTAATCAATTACGGGGTCATTAGTTCATAGCCCATATATGG | 21 |
| promoter, | AGTTCCGCGTTACATAACTTACGGTAAATGGCCCGCCTGGCTGACCGCCCA | |
| 5′UTR and | ACGACCCCCGCCCATTGACGTCAATAATGACGTATGTTCCCATAGTAACGC | |
| leader | CAATAGGGACTTTCCATTGACGTCAATGGGTGGAGTATTTACGGTAAACTGC | |
| sequence, | CCACTTGGCAGTACATCAAGTGTATCATATGCCAAGTACGCCCCCTATTGAC | |
| Spike | GTCAATGACGGTAAATGGCCCGCCTGGCATTATGCCCAGTACATGACCTTA | |
| glyco | TGGGACTTTCCTACTTGGCAGTACATCTACGTATTAGTCATCGCTATTACCA | |
| protein | TGGTGATGCGGTTTTGGCAGTACATCAATGGGCGTGGATAGCGGTTTGACT | |
| (HexaPro- | CACGGGGATTTCCAAGTCTCCACCCCATTGACGTCAATGGGAGTTTGTTTTG | |
| mutations), | GCACCAAAATCAACGGGACTTTCCAAAATGTCGTAACAACTCCGCCCCATTG | |
| linker, | ACGCAAATGGGCGGTAGGCGTGTACGGTGGGAGGTCTATATAAGCAGAGC | |
| HA, Stop | TGGTTTAGTGAACCGTCAGATCGAGAATAAACTAGTATTCTTCTGGTCCCCA | |
| Codon, | CAGACTCAGAGAGAACCCGCCACCATGTTCGTGTTCCTGGTGCTGCTGCCC | |
| 3′UTR and | CTGGTGAGCAGCCAGTGCGTGAACCTGACCACCAGGACCCAGCTGCCCCC | |
| PolyA tail | CGCCTACACCAACAGCTTCACCAGGGGCGTGTACTACCCCGACAAGGTGTT | |
| | CAGGAGCAGCGTGCTGCACAGCACCCAGGACCTGTTCCTGCCCTTCTTCAG | |
| | CAACGTGACCTGGTTCCACGCCATCCACGTGAGCGGCACCAACGGCACCA | |
| | AGAGGTTCGACAACCCCGTGCTGCCCTTCAACGACGGCGTGTACTTCGCCA | |
| | GCACCGAGAAGAGCAACATCATCAGGGGCTGGATCTTCGGCACCACCCTG | |
| | GACAGCAAGACCCAGAGCCTGCTGATCGTGAACAACGCCACCAACGTGGT | |
| | GATCAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTACAG | |
| | CAGCGCCAACAACTGCACCTTCGAGTACGTGAGCCAGCCCTTCCTGATGGA | |
| | CCTGGAGGGCAAGCAGGGCAACTTCAAGAACCTGAGGGAGTTCGTGTTCA | |
| | AGAACATCGACGGCTACTTCAAGATCTACAGCAAGCACACCCCCATCAACC | |
| | TGGTGAGGGACCTGCCCCAGGGCTTCAGCGCCCTGGAGCCCCTGGTGGAC | |
| | CTGCCCATCGGCATCAACATCACCAGGTTCCAGACCCTGCTGGCCCTGCAC | |
| | AGGAGCTACCTGACCCCCGGCGACAGCAGCAGCGGCTGGACCGCCGGCG | |
| | CCGCCGCCTACTACGTGGGCTACCTGCAGCCCAGGACCTTCCTGCTGAAG | |
| | TACAACGAGAACGGCACCATCACCGACGCCGTGGACTGCGCCCTGGACCC | |
| | CCTGAGCGAGACCAAGTGCACCCTGAAGAGCTTCACCGTGGAGAAGGGCA | |
| | TCTACCAGACCAGCAACTTCAGGGTGCAGCCCACCGAGAGCATCGTGAGG | |
| | TTCCCCAACATCACCAACCTGTGCCCCTTCGGCGAGGTGTTCAACGCCACC | |
| | AGGTTCGCCAGCGTGTACGCCTGGAACAGGAAGAGGATCAGCAACTGCGT | |
| | GGCCGACTACAGCGTGCTGTACAACAGCGCCAGCTTCAGCACCTTCAAGTG | |
| | CTACGGCGTGAGCCCCACCAAGCTGAACGACCTGTGCTTCACCAACGTGTA | |
| | CGCCGACAGCTTCGTGATCAGGGGCGACGAGGTGAGGCAGATCGCCCCC | |
| | GGCCAGACCGGCAAGATCGCCGACTACAACTACAAGCTGCCCGACGACTT | |
| | CACCGGCTGCGTGATCGCCTGGAACAGCAACAACCTGGACAGCAAGGTGG | |
| | GCGGCAACTACAACTACCTGTACAGGCTGTTCAGGAAGAGCAACCTGAAGC | |
| | CCTTCGAGAGGGACATCAGCACCGAGATCTACCAGGCCGGCAGCACCCCC | |
| | TGCAACGGCGTGGAGGGCTTCAACTGCTACTTCCCCCTGCAGAGCTACGG | |
| | CTTCCAGCCCACCAACGGCGTGGGCTACCAGCCCTACAGGGTGGTGGTGC | |
| | TGAGCTTCGAGCTGCTGCACGCCCCCGCCACCGTGTGCGGCCCCAAGAAG | |
| | AGCACCAACCTGGTGAAGAACAAGTGCGTGAACTTCAACTTCAACGGCCTG | |
| | ACCGGCACCGGCGTGCTGACCGAGAGCAACAAGAAGTTCCTGCCCTTCCA | |
| | GCAGTTCGGCAGGGACATCGCCGACACCACCGACGCCGTGAGGGACCCC | |
| | CAGACCCTGGAGATCCTGGACATCACCCCCTGCAGCTTCGGCGGCGTGAG | |
| | CGTGATCACCCCCGGCACCAACACCAGCAACCAGGTGGCCGTGCTGTACC | |
| | AGGACGTGAACTGCACCGAGGTGCCCGTGGCCATCCACGCCGACCAGCTG | |
| | ACCCCCACCTGGAGGGTGTACAGCACCGGCAGCAACGTGTTCCAGACCAG | |
| | GGCCGGCTGCCTGATCGGCGCCGAGCACGTGAACAACAGCTACGAGTGCG | |
| | ACATCCCCATCGGCGCCGGCATCTGCGCCAGCTACCAGACCCAGACCAAC | |
| | AGCCCCGGCAGCGCCAGCAGCGTGGCCAGCCAGAGCATCATCGCCTACAC | |
| | CATGAGCCTGGGCGCCGAGAACAGCGTGGCCTACAGCAACAACAGCATCG | |
| | CCATCCCCACCAACTTCACCATCAGCGTGACCACCGAGATCCTGCCCGTGA | |
| | GCATGACCAAGACCAGCGTGGACTGCACCATGTACATCTGCGGCGACAGC | |
| | ACCGAGTGCAGCAACCTGCTGCTGCAGTACGGCAGCTTCTGCACCCAGCT | |
| | GAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACT | |
| | TCGGCGGCTTCAACTTCAGCCAGATCCTGCCCGACCCCAGCAAGCCCAGC | |
| | AAGAGGAGCCCCATCGAGGACCTGCTGTTCAACAAGGTGACCCTGGCCGA | |
| | CGCCGGCTTCATCAAGCAGTACGGCGACTGCCTGGGCGACATCGCCGCCA | |
| | GGGACCTGATCTGCGCCCAGAAGTTCAACGGCCTGACCGTGCTGCCCCCC | |
| | CTGCTGACCGACGAGATGATCGCCCAGTACACCAGCGCCCTGCTGGCCGG | |
| | CACCATCACCAGCGGCTGGACCTTCGGCGCCGGCCCCGCCCTGCAGATCC | |
| | CCTTCCCCATGCAGATGGCCTACAGGTTCAACGGCATCGGCGTGACCCAGA | |
| | ACGTGCTGTACGAGAACCAGAAGCTGATCGCCAACCAGTTCAACAGCGCCA | |
| | TCGGCAAGATCCAGGACAGCCTGAGCAGCACCCCCAGCGCCCTGGGCAAG | |
| | CTGCAGGACGTGGTGAACCAGAACGCCCAGGCCCTGAACACCCTGGTGAA | |
| | GCAGCTGAGCAGCAACTTCGGCGCCATCAGCAGCGTGCTGAACGACATCC | |
| | TGAGCAGGCTGGACCCCCCCGAGGCCGAGGTGCAGATCGACAGGCTGATC | |
| | ACCGGCAGGCTGCAGAGCCTGCAGACCTACGTGACCCAGCAGCTGATCAG | |
| | GGCCGCCGAGATCAGGGCCAGCGCCAACCTGGCCGCCACCAAGATGAGC | |
| | GAGTGCGTGCTGGGCCAGAGCAAGAGGGTGGACTTCTGCGGCAAGGGCTA | |
| | CCACCTGATGAGCTTCCCCCAGAGCGCCCCCCACGGCGTGGTGTTCCTGC | |
| | ACGTGACCTACGTGCCCGCCCAGGAGAAGAACTTCACCACCGCCCCCGCC | |
| | ATCTGCCACGACGGCAAGGCCCACTTCCCCAGGGAGGGCGTGTTCGTGAG | |
| | CAACGGCACCCACTGGTTCGTGACCCAGAGGAACTTCTACGAGCCCCAGAT | |
| | CATCACCACCGACAACACCTTCGTGAGCGGCAACTGCGACGTGGTGATCG | |
| | GCATCGTGAACAACACCGTGTACGACCCCCTGCAGCCCGAGCTGGACAGC | |
| | TTCAAGGAGGAGCTGGACAAGTACTTCAAGAACCACACCAGCCCCGACGTG | |
| | GACCTGGGCGACATCAGCGGCATCAACGCCAGCGTGGTGAACATCCAGAA | |
| | GGAGATCGACAGGCTGAACGAGGTGGCCAAGAACCTGAACGAGAGCCTGA | |
| | TCGACCTGCAGGAGCTGGGCAAGTACGAGCAGTACATCAAGTGGCCCTGG | |
| | TACATCTGGCTGGGCTTCATCGCCGGCCTGATCGCCATCGTGATGGTGACC | |
| | ATCATGCTGTGCTGCATGACCAGCTGCTGCAGCTGCCTGAAGGGCTGCTGC | |
| | AGCTGCGGCAGCTGCTGCAAGTTCGACGAGGACGACAGCGAGCCCGTGCT | |
| | GAAGGGCGTGAAGCTGCACTACACCGGAAGCGGAGCCACGAACTTCTCTC | |
| | TGTTAAAGCAAGCAGGAGATGTTGAAGAAAACCCCGGGCCTTTCGGAGCTA | |
| | TTGCTGGTTTCTTGGAAGGAGGATGGGAAGGAATGATTGCAGGTTGGCAC | |
| | GGATACACATCTCATGGAGCACATGGAGTAGCAGTGGCAGCAGACCTTAA | |
| | GAGTACCCATGATGACTCGAGCTGGTACTGCATGCACGCAATGCTAGCTG | |
| | CCCCTTTCCCGTCCTGGGTACCCCGAGTCTCCCCCGACCTCGGGTCCCAG | |
| | GTATGCTCCCACCTCCACCTGCCCCACTCACCACCTCTGCTAGTTCCAGAC | |
| | ACCTCCCAAGCACGCAGCAATGCAGCTCAAAACGCTTAGCCTAGCCACAC | |
| | CCCCACGGGAAACAGCAGTGATTAACCTTTAGCAATAAACGAAAGTTTAA | |
| | CTAAGCTATACTAACCCCAGGGTTGGTCAATTTCGTGCCAGCCACACCCT | |
| | GGAGCTAGCAAAAAAAA | |
| 2 | TAGTTATTAATAGTAATCAATTACGGGGTCATTAGTTCATAGCCCATATATGG | 22 |
| promoter, | AGTTCCGCGTTACATAACTTACGGTAAATGGCCCGCCTGGCTGACCGCCCA | |
| 5′UTR and | ACGACCCCCGCCCATTGACGTCAATAATGACGTATGTTCCCATAGTAACGC | |
| leader | CAATAGGGACTTTCCATTGACGTCAATGGGTGGAGTATTTACGGTAAACTGC | |
| sequence, | CCACTTGGCAGTACATCAAGTGTATCATATGCCAAGTACGCCCCCTATTGAC | |
| Spike | GTCAATGACGGTAAATGGCCCGCCTGGCATTATGCCCAGTACATGACCTTA | |
| glyco | TGGGACTTTCCTACTTGGCAGTACATCTACGTATTAGTCATCGCTATTACCA | |
| protein | TGGTGATGCGGTTTTGGCAGTACATCAATGGGCGTGGATAGCGGTTTGACT | |
| (two | CACGGGGATTTCCAAGTCTCCACCCCATTGACGTCAATGGGAGTTTGTTTTG | |
| proline | GCACCAAAATCAACGGGACTTTCCAAAATGTCGTAACAACTCCGCCCCATTG | |
| subst.), | ACGCAAATGGGCGGTAGGCGTGTACGGTGGGAGGTCTATATAAGCAGAGC | |
| linker, | TGGTTTAGTGAACCGTCAGATCGAGAATAAACTAGTATTCTTCcTGGTCCCC | |
| HA, Stop | ACAGACTCAGAGAGAACCCGCCACCATGTTCGTGTTCCTGGTGCTGCTGCC | |
| Codon, | CCTGGTGAGCAGCCAGTGCGTGAACCTGACCACCAGGACCCAGCTGCCCC | |
| 3′UTR and | CCGCCTACACCAACAGCTTCACCAGGGGCGTGTACTACCCCGACAAGGTGT | |
| PolyA tail | TCAGGAGCAGCGTGCTGCACAGCACCCAGGACCTGTTCCTGCCCTTCTTCA | |
| | GCAACGTGACCTGGTTCCACGCCATCCACGTGAGCGGCACCAACGGCACC | |
| | AAGAGGTTCGACAACCCCGTGCTGCCCTTCAACGACGGCGTGTACTTCGCC | |
| | AGCACCGAGAAGAGCAACATCATCAGGGGCTGGATCTTCGGCACCACCCT | |
| | GGACAGCAAGACCCAGAGCCTGCTGATCGTGAACAACGCCACCAACGTGG | |
| | TGATCAAGGTGTGCGAGTTCCAGTTCTGCAACGACCCCTTCCTGGGCGTGT | |
| | ACTACCACAAGAACAACAAGAGCTGGATGGAGAGCGAGTTCAGGGTGTACA | |
| | GCAGCGCCAACAACTGCACCTTCGAGTACGTGAGCCAGCCCTTCCTGATGG | |
| | ACCTGGAGGGCAAGCAGGGCAACTTCAAGAACCTGAGGGAGTTCGTGTTC | |
| | AAGAACATCGACGGCTACTTCAAGATCTACAGCAAGCACACCCCCATCAAC | |
| | CTGGTGAGGGACCTGCCCCAGGGCTTCAGCGCCCTGGAGCCCCTGGTGGA | |
| | CCTGCCCATCGGCATCAACATCACCAGGTTCCAGACCCTGCTGGCCCTGCA | |
| | CAGGAGCTACCTGACCCCCGGCGACAGCAGCAGCGGCTGGACCGCCGGC | |
| | GCCGCCGCCTACTACGTGGGCTACCTGCAGCCCAGGACCTTCCTGCTGAA | |
| | GTACAACGAGAACGGCACCATCACCGACGCCGTGGACTGCGCCCTGGACC | |
| | CCCTGAGCGAGACCAAGTGCACCCTGAAGAGCTTCACCGTGGAGAAGGGC | |
| | ATCTACCAGACCAGCAACTTCAGGGTGCAGCCCACCGAGAGCATCGTGAG | |
| | GTTCCCCAACATCACCAACCTGTGCCCCTTCGGCGAGGTGTTCAACGCCAC | |
| | CAGGTTCGCCAGCGTGTACGCCTGGAACAGGAAGAGGATCAGCAACTGCG | |
| | TGGCCGACTACAGCGTGCTGTACAACAGCGCCAGCTTCAGCACCTTCAAGT | |
| | GCTACGGCGTGAGCCCCACCAAGCTGAACGACCTGTGCTTCACCAACGTGT | |
| | ACGCCGACAGCTTCGTGATCAGGGGCGACGAGGTGAGGCAGATCGCCCCC | |
| | GGCCAGACCGGCAAGATCGCCGACTACAACTACAAGCTGCCCGACGACTT | |
| | CACCGGCTGCGTGATCGCCTGGAACAGCAACAACCTGGACAGCAAGGTGG | |
| | GCGGCAACTACAACTACCTGTACAGGCTGTTCAGGAAGAGCAACCTGAAGC | |
| | CCTTCGAGAGGGACATCAGCACCGAGATCTACCAGGCCGGCAGCACCCCC | |
| | TGCAACGGCGTGGAGGGCTTCAACTGCTACTTCCCCCTGCAGAGCTACGG | |
| | CTTCCAGCCCACCAACGGCGTGGGCTACCAGCCCTACAGGGTGGTGGTGC | |
| | TGAGCTTCGAGCTGCTGCACGCCCCCGCCACCGTGTGCGGCCCCAAGAAG | |
| | AGCACCAACCTGGTGAAGAACAAGTGCGTGAACTTCAACTTCAACGGCCTG | |
| | ACCGGCACCGGCGTGCTGACCGAGAGCAACAAGAAGTTCCTGCCCTTCCA | |
| | GCAGTTCGGCAGGGACATCGCCGACACCACCGACGCCGTGAGGGACCCC | |
| | CAGACCCTGGAGATCCTGGACATCACCCCCTGCAGCTTCGGCGGCGTGAG | |
| | CGTGATCACCCCCGGCACCAACACCAGCAACCAGGTGGCCGTGCTGTACC | |
| | AGGACGTGAACTGCACCGAGGTGCCCGTGGCCATCCACGCCGACCAGCTG | |
| | ACCCCCACCTGGAGGGTGTACAGCACCGGCAGCAACGTGTTCCAGACCAG | |
| | GGCCGGCTGCCTGATCGGCGCCGAGCACGTGAACAACAGCTACGAGTGCG | |
| | ACATCCCCATCGGCGCCGGCATCTGCGCCAGCTACCAGACCCAGACCAAC | |
| | AGCCCCAGGAGGGCCAGGAGCGTGGCCAGCCAGAGCATCATCGCCTACAC | |
| | CATGAGCCTGGGCGCCGAGAACAGCGTGGCCTACAGCAACAACAGCATCG | |
| | CCATCCCCACCAACTTCACCATCAGCGTGACCACCGAGATCCTGCCCGTGA | |
| | GCATGACCAAGACCAGCGTGGACTGCACCATGTACATCTGCGGCGACAGC | |
| | ACCGAGTGCAGCAACCTGCTGCTGCAGTACGGCAGCTTCTGCACCCAGCT | |
| | GAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACT | |
| | TCGGCGGCTTCAACTTCAGCCAGATCCTGCCCGACCCCAGCAAGCCCAGC | |
| | AAGAGGAGCTTCATCGAGGACCTGCTGTTCAACAAGGTGACCCTGGCCGAC | |
| | GCCGGCTTCATCAAGCAGTACGGCGACTGCCTGGGCGACATCGCCGCCAG | |
| | GGACCTGATCTGCGCCCAGAAGTTCAACGGCCTGACCGTGCTGCCCCCCC | |
| | TGCTGACCGACGAGATGATCGCCCAGTACACCAGCGCCCTGCTGGCCGGC | |
| | ACCATCACCAGCGGCTGGACCTTCGGCGCCGGCGCCGCCCTGCAGATCCC | |
| | CTTCGCCATGCAGATGGCCTACAGGTTCAACGGCATCGGCGTGACCCAGAA | |
| | CGTGCTGTACGAGAACCAGAAGCTGATCGCCAACCAGTTCAACAGCGCCAT | |
| | CGGCAAGATCCAGGACAGCCTGAGCAGCACCGCCAGCGCCCTGGGCAAG | |
| | CTGCAGGACGTGGTGAACCAGAACGCCCAGGCCCTGAACACCCTGGTGAA | |
| | GCAGCTGAGCAGCAACTTCGGCGCCATCAGCAGCGTGCTGAACGACATCC | |
| | TGAGCAGGCTGGACCCCCCCGAGGCCGAGGTGCAGATCGACAGGCTGATC | |
| | ACCGGCAGGCTGCAGAGCCTGCAGACCTACGTGACCCAGCAGCTGATCAG | |
| | GGCCGCCGAGATCAGGGCCAGCGCCAACCTGGCCGCCACCAAGATGAGC | |
| | GAGTGCGTGCTGGGCCAGAGCAAGAGGGTGGACTTCTGCGGCAAGGGCTA | |
| | CCACCTGATGAGCTTCCCCCAGAGCGCCCCCCACGGCGTGGTGTTCCTGC | |
| | ACGTGACCTACGTGCCCGCCCAGGAGAAGAACTTCACCACCGCCCCCGCC | |
| | ATCTGCCACGACGGCAAGGCCCACTTCCCCAGGGAGGGCGTGTTCGTGAG | |
| | CAACGGCACCCACTGGTTCGTGACCCAGAGGAACTTCTACGAGCCCCAGAT | |
| | CATCACCACCGACAACACCTTCGTGAGCGGCAACTGCGACGTGGTGATCG | |
| | GCATCGTGAACAACACCGTGTACGACCCCCTGCAGCCCGAGCTGGACAGC | |
| | TTCAAGGAGGAGCTGGACAAGTACTTCAAGAACCACACCAGCCCCGACGTG | |
| | GACCTGGGCGACATCAGCGGCATCAACGCCAGCGTGGTGAACATCCAGAA | |
| | GGAGATCGACAGGCTGAACGAGGTGGCCAAGAACCTGAACGAGAGCCTGA | |
| | TCGACCTGCAGGAGCTGGGCAAGTACGAGCAGTACATCAAGTGGCCCTGG | |
| | TACATCTGGCTGGGCTTCATCGCCGGCCTGATCGCCATCGTGATGGTGACC | |
| | ATCATGCTGTGCTGCATGACCAGCTGCTGCAGCTGCCTGAAGGGCTGCTGC | |
| | AGCTGCGGCAGCTGCTGCAAGTTCGACGAGGACGACAGCGAGCCCGTGCT | |
| | GAAGGGCGTGAAGCTGCACTACACCGGAAGCGGAGCCACGAACTTCTCTC | |
| | TGTTAAAGCAAGCAGGAGATGTTGAAGAAAACCCCGGGCCTTTCGGAGCTA | |
| | TTGCTGGTTTCTTGGAAGGAGGATGGGAAGGAATGATTGCAGGTTGGCAC | |
| | GGATACACATCTCATGGAGCACATGGAGTAGCAGTGGCAGCAGACCTTAA | |
| | GAGTACCCATGATGACTCGAGCTGGTACTGCATGCACGCAATGCTAGCTG | |
| | CCCCTTTCCCGTCCTGGGTACCCCGAGTCTCCCCCGACCTCGGGTCCCAG | |
| | GTATGCTCCCACCTCCACCTGCCCCACTCACCACCTCTGCTAGTTCCAGAC | |
| | ACCTCCCAAGCACGCAGCAATGCAGCTCAAAACGCTTAGCCTAGCCACAC | |
| | CCCCACGGGAAACAGCAGTGATTAACCTTTAGCAATAAACGAAAGTTTAA | |
| | CTAAGCTATACTAACCCCAGGGTTGGTCAATTTCGTGCCAGCCACACCCT | |
| | GGAGCTAGCAAAAAAAA | |
| 3 | CTCGACATTGATTATTGACTAGTTATTAATAGTAATCAATTACGGGGTCATTA | 23 |
| promoter, | GTTCATAGCCCATATATGGAGTTCCGCGTTACATAACTTACGGTAAATGGCC | |
| 5′UTR and | CGCCTGGCTGACCGCCCAACGACCCCCGCCCATTGACGTCAATAATGACGT | |
| leader | ATGTTCCCATAGTAACGCCAATAGGGACTTTCCATTGACGTCAATGGGTGGA | |
| sequence, | GTATTTACGGTAAACTGCCCACTTGGCAGTACATCAAGTGTATCATATGCCA | |
| Spike | AGTACGCCCCCTATTGACGTCAATGACGGTAAATGGCCCGCCTGGCATTAT | |
| glyco | GCCCAGTACATGACCTTATGGGACTTTCCTACTTGGCAGTACATCTACGTAT | |
| protein | TAGTCATCGCTATTACCATGGTCGAGGTGAGCCCCACGTTCTGCTTCACTCT | |
| (HexaPro- | CCCCATCTCCCCCCCCTCCCCACCCCCAATTTTGTATTTATTTATTTTTTAAT | |
| mutations), | TATTTTGTGCAGCGATGGGGGCGGGGGGGGGGGGGGGGCGCGCGCCAG | |
| linker, | GCGGGGCGGGGCGGGGCGAGGGGCGGGGCGGGGCGAGGCGGAGAGGT | |
| HA, Stop | GCGGCGGCAGCCAATCAGAGCGGCGCGCTCCGAAAGTTTCCTTTTATGGC | |
| Codon, | GAGGCGGCGGCGGCGGCGGCCCTATAAAAAGCGAAGCGCGCGGGGGC | |
| 3′UTR and | GGGAGTCGCTGCGCGCTGCCTTCGCCCCGTGCCCCGCTCCGCCGCCGCC | |
| PolyA tail | TCGCGCCGCCCGCCCCGGCTCTGACTGACCGCGTTACTCCCACAGGTGAG | |
| | CGGGCGGGACGGCCCTTCTCCTCCGGGCTGTAATTAGCGCTTGGTTTAATG | |
| | ACGGCTTGTTTCTTTTCTGTGGCTGCGTGAAAGCCTTGAGGGGCTCCGGGA | |
| | GGGCCCTTTGTGGGGGGGGAGCGGCTCGGGGGGTGCGTGCGTGTGTGTG | |
| | TGCGTGGGGAGCGCCGCGTGCGGCTCCGCGCTGCCCGGCGGCTGTGAGC | |
| | GCTGCGGGCGCGGCGCGGGGCTTTGTGCGCTCCGCAGTGTGCGCGAGGG | |
| | GAGCGCGGCCGGGGGGGGTGCCCCGCGGTGCGGGGGGGGCTGCGAGGG | |
| | GAACAAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCCCGAGT | |
| | TGCTGAGCACGGCCCGGCTTCGGGTGCGGGGCTCCGTACGGGGCGTGGC | |
| | GCGGGGCTCGCCGTGCCGGGCGGGGGGTGGCGGCAGGTGGGGGTGCCG | |
| | GGCGGGGCGGGGCCCGCCTCGGGCCGGGGAGGGCTCGGGGGAGGGGCG | |
| | CGGCGGCCCCCGGAGCGCCGGCGGCTGTCGAGGCGCGGCGAGCCGCAG | |
| | CCATTGCCTTTTATGGTAATCGTGCGAGAGGGCGCAGGGACTTCCTTTGTC | |
| | CCAAATCTGTGCGGAGCCGAAATCTGGGAGGCGCCGCCGCACCCCCTCTA | |
| | GCGGGCGCGGGGCGAAGCGGTGCGGCGCCGGCAGGAAGGAAATGGGCG | |
| | GGGAGGGCCTTCGTGCGTCGCCGCGCCGCCGTCCCCTTCTCCCTCTCCAG | |
| | CCTCGGGGCTGTCCGCGGGGGGACGGCTGCCTTCGGGGGGGACGGGGC | |
| | AGGGCGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGTGCTG | |
| | GTTATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTTCCT | |
| | GGTGCTGCTGCCCCTGGTGAGCAGCCAGTGCGTGAACCTGACCACCAGGA | |
| | CCCAGCTGCCCCCCGCCTACACCAACAGCTTCACCAGGGGCGTGTACTAC | |
| | CCCGACAAGGTGTTCAGGAGCAGCGTGCTGCACAGCACCCAGGACCTGTT | |
| | CCTGCCCTTCTTCAGCAACGTGACCTGGTTCCACGCCATCCACGTGAGCGG | |
| | CACCAACGGCACCAAGAGGTTCGACAACCCCGTGCTGCCCTTCAACGACG | |
| | GCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAAC | |
| | GCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAG | |
| | TTCAGGGTGTACAGCAGCGCCAACAACTGCACCTTCGAGTACGTGAGCCAG | |
| | CCCTTCCTGATGGACCTGGAGGGCAAGCAGGGCAACTTCAAGAACCTGAG | |
| | GGAGTTCGTGTTCAAGAACATCGACGGCTACTTCAAGATCTACAGCAAGCA | |
| | CACCCCCATCAACCTGGTGAGGGACCTGCCCCAGGGCTTCAGCGCCCTGG | |
| | AGCCCCTGGTGGACCTGCCCATCGGCATCAACATCACCAGGTTCCAGACCC | |
| | TGCTGGCCCTGCACAGGAGCTACCTGACCCCCGGCGACAGCAGCAGCGGC | |
| | TGGACCGCCGGCGCCGCCGCCTACTACGTGGGCTACCTGCAGCCCAGGAC | |
| | CTTCCTGCTGAAGTACAACGAGAACGGCACCATCACCGACGCCGTGGACTG | |
| | CGCCCTGGACCCCCTGAGCGAGACCAAGTGCACCCTGAAGAGCTTCACCG | |
| | TGGAGAAGGGCATCTACCAGACCAGCAACTTCAGGGTGCAGCCCACCGAG | |
| | AGCATCGTGAGGTTCCCCAACATCACCAACCTGTGCCCCTTCGGCGAGGTG | |
| | TTCAACGCCACCAGGTTCGCCAGCGTGTACGCCTGGAACAGGAAGAGGAT | |
| | CAGCAACTGCGTGGCCGACTACAGCGTGCTGTACAACAGCGCCAGCTTCA | |
| | GCACCTTCAAGTGCTACGGCGTGAGCCCCACCAAGCTGAACGACCTGTGCT | |
| | TCACCAACGTGTACGCCGACAGCTTCGTGATCAGGGGCGACGAGGTGAGG | |
| | CAGATCGCCCCCGGCCAGACCGGCAAGATCGCCGACTACAACTACAAGCT | |
| | GCCCGACGACTTCACCGGCTGCGTGATCGCCTGGAACAGCAACAACCTGG | |
| | ACAGCAAGGTGGGCGGCAACTACAACTACCTGTACAGGCTGTTCAGGAAGA | |
| | GCAACCTGAAGCCCTTCGAGAGGGACATCAGCACCGAGATCTACCAGGCC | |
| | GGCAGCACCCCCTGCAACGGCGTGGAGGGCTTCAACTGCTACTTCCCCCT | |
| | GCAGAGCTACGGCTTCCAGCCCACCAACGGCGTGGGCTACCAGCCCTACA | |
| | GGGTGGTGGTGCTGAGCTTCGAGCTGCTGCACGCCCCCGCCACCGTGTGC | |
| | GGCCCCAAGAAGAGCACCAACCTGGTGAAGAACAAGTGCGTGAACTTCAAC | |
| | TTCAACGGCCTGACCGGCACCGGCGTGCTGACCGAGAGCAACAAGAAGTT | |
| | CCTGCCCTTCCAGCAGTTCGGCAGGGACATCGCCGACACCACCGACGCCG | |
| | TGAGGGACCCCCAGACCCTGGAGATCCTGGACATCACCCCCTGCAGCTTC | |
| | GGCGGCGTGAGCGTGATCACCCCCGGCACCAACACCAGCAACCAGGTGGC | |
| | CGTGCTGTACCAGGACGTGAACTGCACCGAGGTGCCCGTGGCCATCCACG | |
| | CCGACCAGCTGACCCCCACCTGGAGGGTGTACAGCACCGGCAGCAACGTG | |
| | TTCCAGACCAGGGCCGGCTGCCTGATCGGCGCCGAGCACGTGAACAACAG | |
| | CTACGAGTGCGACATCCCCATCGGCGCCGGCATCTGCGCCAGCTACCAGA | |
| | CCCAGACCAACAGCCCCGGCAGCGCCAGCAGCGTGGCCAGCCAGAGCAT | |
| | CATCGCCTACACCATGAGCCTGGGCGCCGAGAACAGCGTGGCCTACAGCA | |
| | ACAACAGCATCGCCATCCCCACCAACTTCACCATCAGCGTGACCACCGAGA | |
| | TCCTGCCCGTGAGCATGACCAAGACCAGCGTGGACTGCACCATGTACATCT | |
| | GCGGCGACAGCACCGAGTGCAGCAACCTGCTGCTGCAGTACGGCAGCTTC | |
| | TGCACCCAGCTGAACAGGGCCCTGACCGGCATCGCCGTGGAGCAGGACAA | |
| | GAACACCCAGGAGGTGTTCGCCCAGGTGAAGCAGATCTACAAGACCCCCC | |
| | CCATCAAGGACTTCGGCGGCTTCAACTTCAGCCAGATCCTGCCCGACCCCA | |
| | GCAAGCCCAGCAAGAGGAGCCCCATCGAGGACCTGCTGTTCAACAAGGTG | |
| | ACCCTGGCCGACGCCGGCTTCATCAAGCAGTACGGCGACTGCCTGGGCGA | |
| | CATCGCCGCCAGGGACCTGATCTGCGCCCAGAAGTTCAACGGCCTGACCG | |
| | TGCTGCCCCCCCTGCTGACCGACGAGATGATCGCCCAGTACACCAGCGCC | |
| | CTGCTGGCCGGCACCATCACCAGCGGCTGGACCTTCGGCGCCGGCCCCG | |
| | CCCTGCAGATCCCCTTCCCCATGCAGATGGCCTACAGGTTCAACGGCATCG | |
| | GCGTGACCCAGAACGTGCTGTACGAGAACCAGAAGCTGATCGCCAACCAG | |
| | TTCAACAGCGCCATCGGCAAGATCCAGGACAGCCTGAGCAGCACCCCCAG | |
| | CGCCCTGGGCAAGCTGCAGGACGTGGTGAACCAGAACGCCCAGGCCCTGA | |
| | ACACCCTGGTGAAGCAGCTGAGCAGCAACTTCGGCGCCATCAGCAGCGTG | |
| | CTGAACGACATCCTGAGCAGGCTGGACCCCCCCGAGGCCGAGGTGCAGAT | |
| | CGACAGGCTGATCACCGGCAGGCTGCAGAGCCTGCAGACCTACGTGACCC | |
| | AGCAGCTGATCAGGGCCGCCGAGATCAGGGCCAGCGCCAACCTGGCCGC | |
| | CACCAAGATGAGCGAGTGCGTGCTGGGCCAGAGCAAGAGGGTGGACTTCT | |
| | GCGGCAAGGGCTACCACCTGATGAGCTTCCCCCAGAGCGCCCCCCACGGC | |
| | GTGGTGTTCCTGCACGTGACCTACGTGCCCGCCCAGGAGAAGAACTTCACC | |
| | ACCGCCCCCGCCATCTGCCACGACGGCAAGGCCCACTTCCCCAGGGAGGG | |
| | CGTGTTCGTGAGCAACGGCACCCACTGGTTCGTGACCCAGAGGAACTTCTA | |
| | CGAGCCCCAGATCATCACCACCGACAACACCTTCGTGAGCGGCAACTGCG | |
| | ACGTGGTGATCGGCATCGTGAACAACACCGTGTACGACCCCCTGCAGCCC | |
| | GAGCTGGACAGCTTCAAGGAGGAGCTGGACAAGTACTTCAAGAACCACACC | |
| | AGCCCCGACGTGGACCTGGGCGACATCAGCGGCATCAACGCCAGCGTGGT | |
| | GAACATCCAGAAGGAGATCGACAGGCTGAACGAGGTGGCCAAGAACCTGA | |
| | ACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATC | |
| | GTGATGGTGACCATCATGCTGTGCTGCATGACCAGCTGCTGCAGCTGCCTG | |
| | AAGGGCTGCTGCAGCTGCGGCAGCTGCTGCAAGTTCGACGAGGACGACAG | |
| | CGAGCCCGTGCTGAAGGGCGTGAAGCTGCACTACACCGGAAGCGGAGCCA | |
| | CGAACTTCTCTCTGTTAAAGCAAGCAGGAGATGTTGAAGAAAACCCCGGGC | |
| | CTTTCGGAGCTATTGCTGGTTTCTTGGAAGGAGGATGGGAAGGAATGATT | |
| | GCAGGTTGGCACGGATACACATCTCATGGAGCACATGGAGTAGCAGTGG | |
| | CAGCAGACCTTAAGAGTACCCATGATGACTCGAGCTGGTACTGCATGCAC | |
| | GCAATGCTAGCTGCCCCTTTCCCGTCCTGGGTACCCCGAGTCTCCCCCGA | |
| | CCTCGGGTCCCAGGTATGCTCCCACCTCCACCTGCCCCACTCACCACCTC | |
| | TGCTAGTTCCAGACACCTCCCAAGCACGCAGCAATGCAGCTCAAAACGCT | |
| | TAGCCTAGCCACACCCCCACGGGAAACAGCAGTGATTAACCTTTAGCAAT | |
| | AAACGAAAGTTTAACTAAGCTATACTAACCCCAGGGTTGGTCAATTTCGTG | |
| | CCAGCCACACCCTGGAGCTAGCAAAAAAAA | |
| 4 | CTCGACATTGATTATTGACTAGTTATTAATAGTAATCAATTACGGGGTCATTA | 24 |
| promoter, | GTTCATAGCCCATATATGGAGTTCCGCGTTACATAACTTACGGTAAATGGCC | |
| 5′UTR and | CGCCTGGCTGACCGCCCAACGACCCCCGCCCATTGACGTCAATAATGACGT | |
| leader | ATGTTCCCATAGTAACGCCAATAGGGACTTTCCATTGACGTCAATGGGTGGA | |
| sequence, | GTATTTACGGTAAACTGCCCACTTGGCAGTACATCAAGTGTATCATATGCCA | |
| Spike | AGTACGCCCCCTATTGACGTCAATGACGGTAAATGGCCCGCCTGGCATTAT | |
| glyco | GCCCAGTACATGACCTTATGGGACTTTCCTACTTGGCAGTACATCTACGTAT | |
| protein | TAGTCATCGCTATTACCATGGTCGAGGTGAGCCCCACGTTCTGCTTCACTCT | |
| (two | CCCCATCTCCCCCCCCTCCCCACCCCCAATTTTGTATTTATTTATTTTTTAAT | |
| proline | TATTTTGTGCAGCGATGGGGGCGGGGGGGGGGGGGGGGCGCGCGCCAG | |
| subst.), | GCGGGGCGGGGCGGGGCGAGGGGCGGGGCGGGGCGAGGCGGAGAGGT | |
| linker, | GCGGCGGCAGCCAATCAGAGCGGCGCGCTCCGAAAGTTTCCTTTTATGGC | |
| HA, Stop | GAGGCGGCGGCGGCGGCGGCCCTATAAAAAGCGAAGCGCGCGGCGGGC | |
| Codon, | GGGAGTCGCTGCGCGCTGCCTTCGCCCCGTGCCCCGCTCCGCCGCCGCC | |
| 3′UTR and | TCGCGCCGCCCGCCCCGGCTCTGACTGACCGCGTTACTCCCACAGGTGAG | |
| PolyA tail | CGGGCGGGACGGCCCTTCTCCTCCGGGCTGTAATTAGCGCTTGGTTTAATG | |
| | ACGGCTTGTTTCTTTTCTGTGGCTGCGTGAAAGCCTTGAGGGGCTCCGGGA | |
| | GGGCCCTTTGTGGGGGGGGAGCGGCTCGGGGGGTGCGTGCGTGTGTGTG | |
| | TGCGTGGGGAGCGCCGCGTGCGGCTCCGCGCTGCCCGGCGGCTGTGAGC | |
| | GCTGCGGGCGCGGCGCGGGGCTTTGTGCGCTCCGCAGTGTGCGCGAGGG | |
| | GAGCGCGGCCGGGGGCGGTGCCCCGCGGTGCGGGGGGGGCTGCGAGGG | |
| | GAACAAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCCCGAGT | |
| | TGCTGAGCACGGCCCGGCTTCGGGTGCGGGGCTCCGTACGGGGCGTGGC | |
| | GCGGGGCTCGCCGTGCCGGGCGGGGGGTGGCGGCAGGTGGGGGTGCCG | |
| | GGCGGGGCGGGGCCGCCTCGGGCCGGGGAGGGCTCGGGGGAGGGGCG | |
| | CGGCGGCCCCCGGAGCGCCGGCGGCTGTCGAGGCGCGGCGAGCCGCAG | |
| | CCATTGCCTTTTATGGTAATCGTGCGAGAGGGCGCAGGGACTTCCTTTGTC | |
| | CCAAATCTGTGCGGAGCCGAAATCTGGGAGGCGCCGCCGCACCCCCTCTA | |
| | GCGGGCGCGGGGCGAAGCGGTGCGGCGCCGGCAGGAAGGAAATGGGCG | |
| | GGGAGGGCCTTCGTGCGTCGCCGCGCCGCCGTCCCCTTCTCCCTCTCCAG | |
| | CCTCGGGGCTGTCCGCGGGGGGACGGCTGCCTTCGGGGGGGACGGGGC | |
| | AGGGCGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGTGCTG | |
| | GTTATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTTCCT | |
| | GGTGCTGCTGCCCCTGGTGAGCAGCCAGTGCGTGAACCTGACCACCAGGA | |
| | CCCAGCTGCCCCCCGCCTACACCAACAGCTTCACCAGGGGCGTGTACTAC | |
| | CCCGACAAGGTGTTCAGGAGCAGCGTGCTGCACAGCACCCAGGACCTGTT | |
| | CCTGCCCTTCTTCAGCAACGTGACCTGGTTCCACGCCATCCACGTGAGCGG | |
| | CACCAACGGCACCAAGAGGTTCGACAACCCCGTGCTGCCCTTCAACGACG | |
| | GCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAAC | |
| | GCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAG | |
| | TTCAGGGTGTACAGCAGCGCCAACAACTGCACCTTCGAGTACGTGAGCCAG | |
| | CCCTTCCTGATGGACCTGGAGGGCAAGCAGGGCAACTTCAAGAACCTGAG | |
| | GGAGTTCGTGTTCAAGAACATCGACGGCTACTTCAAGATCTACAGCAAGCA | |
| | CACCCCCATCAACCTGGTGAGGGACCTGCCCCAGGGCTTCAGCGCCCTGG | |
| | AGCCCCTGGTGGACCTGCCCATCGGCATCAACATCACCAGGTTCCAGACCC | |
| | TGCTGGCCCTGCACAGGAGCTACCTGACCCCCGGCGACAGCAGCAGCGGC | |
| | TGGACCGCCGGCGCCGCCGCCTACTACGTGGGCTACCTGCAGCCCAGGAC | |
| | CTTCCTGCTGAAGTACAACGAGAACGGCACCATCACCGACGCCGTGGACTG | |
| | CGCCCTGGACCCCCTGAGCGAGACCAAGTGCACCCTGAAGAGCTTCACCG | |
| | TGGAGAAGGGCATCTACCAGACCAGCAACTTCAGGGTGCAGCCCACCGAG | |
| | AGCATCGTGAGGTTCCCCAACATCACCAACCTGTGCCCCTTCGGCGAGGTG | |
| | TTCAACGCCACCAGGTTCGCCAGCGTGTACGCCTGGAACAGGAAGAGGAT | |
| | CAGCAACTGCGTGGCCGACTACAGCGTGCTGTACAACAGCGCCAGCTTCA | |
| | GCACCTTCAAGTGCTACGGCGTGAGCCCCACCAAGCTGAACGACCTGTGCT | |
| | TCACCAACGTGTACGCCGACAGCTTCGTGATCAGGGGCGACGAGGTGAGG | |
| | CAGATCGCCCCCGGCCAGACCGGCAAGATCGCCGACTACAACTACAAGCT | |
| | GCCCGACGACTTCACCGGCTGCGTGATCGCCTGGAACAGCAACAACCTGG | |
| | ACAGCAAGGTGGGCGGCAACTACAACTACCTGTACAGGCTGTTCAGGAAGA | |
| | GCAACCTGAAGCCCTTCGAGAGGGACATCAGCACCGAGATCTACCAGGCC | |
| | GGCAGCACCCCCTGCAACGGCGTGGAGGGCTTCAACTGCTACTTCCCCCT | |
| | GCAGAGCTACGGCTTCCAGCCCACCAACGGCGTGGGCTACCAGCCCTACA | |
| | GGGTGGTGGTGCTGAGCTTCGAGCTGCTGCACGCCCCCGCCACCGTGTGC | |
| | GGCCCCAAGAAGAGCACCAACCTGGTGAAGAACAAGTGCGTGAACTTCAAC | |
| | TTCAACGGCCTGACCGGCACCGGCGTGCTGACCGAGAGCAACAAGAAGTT | |
| | CCTGCCCTTCCAGCAGTTCGGCAGGGACATCGCCGACACCACCGACGCCG | |
| | TGAGGGACCCCCAGACCCTGGAGATCCTGGACATCACCCCCTGCAGCTTC | |
| | GGCGGCGTGAGCGTGATCACCCCCGGCACCAACACCAGCAACCAGGTGGC | |
| | CGTGCTGTACCAGGACGTGAACTGCACCGAGGTGCCCGTGGCCATCCACG | |
| | CCGACCAGCTGACCCCCACCTGGAGGGTGTACAGCACCGGCAGCAACGTG | |
| | TTCCAGACCAGGGCCGGCTGCCTGATCGGCGCCGAGCACGTGAACAACAG | |
| | CTACGAGTGCGACATCCCCATCGGCGCCGGCATCTGCGCCAGCTACCAGA | |
| | CCCAGACCAACAGCCCCAGGAGGGCCAGGAGCGTGGCCAGCCAGAGCAT | |
| | CATCGCCTACACCATGAGCCTGGGCGCCGAGAACAGCGTGGCCTACAGCA | |
| | ACAACAGCATCGCCATCCCCACCAACTTCACCATCAGCGTGACCACCGAGA | |
| | TCCTGCCCGTGAGCATGACCAAGACCAGCGTGGACTGCACCATGTACATCT | |
| | GCGGCGACAGCACCGAGTGCAGCAACCTGCTGCTGCAGTACGGCAGCTTC | |
| | TGCACCCAGCTGAACAGGGCCCTGACCGGCATCGCCGTGGAGCAGGACAA | |
| | GAACACCCAGGAGGTGTTCGCCCAGGTGAAGCAGATCTACAAGACCCCCC | |
| | CCATCAAGGACTTCGGCGGCTTCAACTTCAGCCAGATCCTGCCCGACCCCA | |
| | GCAAGCCCAGCAAGAGGAGCTTCATCGAGGACCTGCTGTTCAACAAGGTGA | |
| | CCCTGGCCGACGCCGGCTTCATCAAGCAGTACGGCGACTGCCTGGGCGAC | |
| | ATCGCCGCCAGGGACCTGATCTGCGCCCAGAAGTTCAACGGCCTGACCGT | |
| | GCTGCCCCCCCTGCTGACCGACGAGATGATCGCCCAGTACACCAGCGCCC | |
| | TGCTGGCCGGCACCATCACCAGCGGCTGGACCTTCGGCGCCGGCGCCGC | |
| | CCTGCAGATCCCCTTCGCCATGCAGATGGCCTACAGGTTCAACGGCATCGG | |
| | CGTGACCCAGAACGTGCTGTACGAGAACCAGAAGCTGATCGCCAACCAGTT | |
| | CAACAGCGCCATCGGCAAGATCCAGGACAGCCTGAGCAGCACCGCCAGCG | |
| | CCCTGGGCAAGCTGCAGGACGTGGTGAACCAGAACGCCCAGGCCCTGAAC | |
| | ACCCTGGTGAAGCAGCTGAGCAGCAACTTCGGCGCCATCAGCAGCGTGCT | |
| | GAACGACATCCTGAGCAGGCTGGACCCCCCCGAGGCCGAGGTGCAGATCG | |
| | ACAGGCTGATCACCGGCAGGCTGCAGAGCCTGCAGACCTACGTGACCCAG | |
| | CAGCTGATCAGGGCCGCCGAGATCAGGGCCAGCGCCAACCTGGCCGCCA | |
| | CCAAGATGAGCGAGTGCGTGCTGGGCCAGAGCAAGAGGGTGGACTTCTGC | |
| | GGCAAGGGCTACCACCTGATGAGCTTCCCCCAGAGCGCCCCCCACGGCGT | |
| | GGTGTTCCTGCACGTGACCTACGTGCCCGCCCAGGAGAAGAACTTCACCAC | |
| | CGCCCCCGCCATCTGCCACGACGGCAAGGCCCACTTCCCCAGGGAGGGC | |
| | GTGTTCGTGAGCAACGGCACCCACTGGTTCGTGACCCAGAGGAACTTCTAC | |
| | GAGCCCCAGATCATCACCACCGACAACACCTTCGTGAGCGGCAACTGCGA | |
| | CGTGGTGATCGGCATCGTGAACAACACCGTGTACGACCCCCTGCAGCCCG | |
| | AGCTGGACAGCTTCAAGGAGGAGCTGGACAAGTACTTCAAGAACCACACCA | |
| | GCCCCGACGTGGACCTGGGCGACATCAGCGGCATCAACGCCAGCGTGGTG | |
| | AACATCCAGAAGGAGATCGACAGGCTGAACGAGGTGGCCAAGAACCTGAA | |
| | CGAGAGCCTGATCGACCTGCAGGAGCTGGGCAAGTACGAGCAGTACATCA | |
| | AGTGGCCCTGGTACATCTGGCTGGGCTTCATCGCCGGCCTGATCGCCATC | |
| | GTGATGGTGACCATCATGCTGTGCTGCATGACCAGCTGCTGCAGCTGCCTG | |
| | AAGGGCTGCTGCAGCTGCGGCAGCTGCTGCAAGTTCGACGAGGACGACAG | |
| | CGAGCCCGTGCTGAAGGGCGTGAAGCTGCACTACACCGGAAGCGGAGCCA | |
| | CGAACTTCTCTCTGTTAAAGCAAGCAGGAGATGTTGAAGAAAACCCCGGGC | |
| | CTTTCGGAGCTATTGCTGGTTTCTTGGAAGGAGGATGGGAAGGAATGATT | |
| | GCAGGTTGGCACGGATACACATCTCATGGAGCACATGGAGTAGCAGTGG | |
| | CAGCAGACCTTAAGAGTACCCATGATGACTCGAGCTGGTACTGCATGCAC | |
| | GCAATGCTAGCTGCCCCTTTCCCGTCCTGGGTACCCCGAGTCTCCCCCGA | |
| | CCTCGGGTCCCAGGTATGCTCCCACCTCCACCTGCCCCACTCACCACCTC | |
| | TGCTAGTTCCAGACACCTCCCAAGCACGCAGCAATGCAGCTCAAAACGCT | |
| | TAGCCTAGCCACACCCCCACGGGAAACAGCAGTGATTAACCTTTAGCAAT | |
| | AAACGAAAGTTTAACTAAGCTATACTAACCCCAGGGTTGGTCAATTTCGTG | |
| | CCAGCCACACCCTGGAGCTAGCAAAAAAAA | |
| 5 | ATGTTCGTGTTCCTGGTGCTGCTGCCCCTGGTGAGCAGCCAGTGCGTGAAC | 25 |
| leader | CTGACCACCAGGACCCAGCTGCCCCCCGCCTACACCAACAGCTTCACCAG | |
| sequence, | GGGCGTGTACTACCCCGACAAGGTGTTCAGGAGCAGCGTGCTGCACAGCA | |
| Spike | CCCAGGACCTGTTCCTGCCCTTCTTCAGCAACGTGACCTGGTTCCACGCCA | |
| glyco | TCCACGTGAGCGGCACCAACGGCACCAAGAGGTTCGACAACCCCGTGCTG | |
| protein | CCCTTCAACGACGGCGTGTACTTCGCCAGCACCGAGAAGAGCAACATCATC | |
| (HexaPro- | AGGGGCTGGATCTTCGGCACCACCCTGGACAGCAAGACCCAGAGCCTGCT | |
| mutations), | GATCGTGAACAACGCCACCAACGTGGTGATCAAGGTGTGCGAGTTCCAGTT | |
| linker, | CTGCAACGACCCCTTCCTGGGCGTGTACTACCACAAGAACAACAAGAGCTG | |
| HA, Stop | GATGGAGAGCGAGTTCAGGGTGTACAGCAGCGCCAACAACTGCACCTTCG | |
| Codon | AGTACGTGAGCCAGCCCTTCCTGATGGACCTGGAGGGCAAGCAGGGCAAC | |
| | TTCAAGAACCTGAGGGAGTTCGTGTTCAAGAACATCGACGGCTACTTCAAG | |
| | ATCTACAGCAAGCACACCCCCATCAACCTGGTGAGGGACCTGCCCCAGGG | |
| | CTTCAGCGCCCTGGAGCCCCTGGTGGACCTGCCCATCGGCATCAACATCA | |
| | CCAGGTTCCAGACCCTGCTGGCCCTGCACAGGAGCTACCTGACCCCCGGC | |
| | GACAGCAGCAGCGGCTGGACCGCCGGCGCCGCCGCCTACTACGTGGGCT | |
| | ACCTGCAGCCCAGGACCTTCCTGCTGAAGTACAACGAGAACGGCACCATCA | |
| | CCGACGCCGTGGACTGCGCCCTGGACCCCCTGAGCGAGACCAAGTGCACC | |
| | CTGAAGAGCTTCACCGTGGAGAAGGGCATCTACCAGACCAGCAACTTCAGG | |
| | GTGCAGCCCACCGAGAGCATCGTGAGGTTCCCCAACATCACCAACCTGTGC | |
| | CCCTTCGGCGAGGTGTTCAACGCCACCAGGTTCGCCAGCGTGTACGCCTG | |
| | GAACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCAAG | |
| | CTGAACGACCTGTGCTTCACCAACGTGTACGCCGACAGCTTCGTGATCAGG | |
| | GGCGACGAGGTGAGGCAGATCGCCCCCGGCCAGACCGGCAAGATCGCCG | |
| | ACTACAACTACAAGCTGCCCGACGACTTCACCGGCTGCGTGATCGCCTGGA | |
| | ACAGCAACAACCTGGACAGCAAGGTGGGGGGCAACTACAACTACCTGTACA | |
| | GGCTGTTCAGGAAGAGCAACCTGAAGCCCTTCGAGAGGGACATCAGCACC | |
| | GAGATCTACCAGGCCGGCAGCACCCCCTGCAACGGCGTGGAGGGCTTCAA | |
| | CTGCTACTTCCCCCTGCAGAGCTACGGCTTCCAGCCCACCAACGGCGTGG | |
| | GCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAA | |
| | GTGCGTGAACTTCAACTTCAACGGCCTGACCGGCACCGGCGTGCTGACCG | |
| | AGAGCAACAAGAAGTTCCTGCCCTTCCAGCAGTTCGGCAGGGACATCGCC | |
| | GACACCACCGACGCCGTGAGGGACCCCCAGACCCTGGAGATCCTGGACAT | |
| | CACCCCCTGCAGCTTCGGCGGCGTGAGCGTGATCACCCCCGGCACCAACA | |
| | CCAGCAACCAGGTGGCCGTGCTGTACCAGGACGTGAACTGCACCGAGGTG | |
| | CCCGTGGCCATCCACGCCGACCAGCTGACCCCCACCTGGAGGGTGTACAG | |
| | CACCGGCAGCAACGTGTTCCAGACCAGGGCCGGCTGCCTGATCGGCGCCG | |
| | AGCACGTGAACAACAGCTACGAGTGCGACATCCCCATCGGCGCCGGCATC | |
| | TGCGCCAGCTACCAGACCCAGACCAACAGCCCCGGCAGCGCCAGCAGCGT | |
| | GGCCAGCCAGAGCATCATCGCCTACACCATGAGCCTGGGCGCCGAGAACA | |
| | GCGTGGCCTACAGCAACAACAGCATCGCCATCCCCACCAACTTCACCATCA | |
| | GCGTGACCACCGAGATCCTGCCCGTGAGCATGACCAAGACCAGCGTGGAC | |
| | TGCACCATGTACATCTGCGGCGACAGCACCGAGTGCAGCAACCTGCTGCT | |
| | GCAGTACGGCAGCTTCTGCACCCAGCTGAACAGGGCCCTGACCGGCATCG | |
| | CCGTGGAGCAGGACAAGAACACCCAGGAGGTGTTCGCCCAGGTGAAGCAG | |
| | ATCTACAAGACCCCCCCCATCAAGGACTTCGGCGGCTTCAACTTCAGCCAG | |
| | ATCCTGCCCGACCCCAGCAAGCCCAGCAAGAGGAGCCCCATCGAGGACCT | |
| | GCTGTTCAACAAGGTGACCCTGGCCGACGCCGGCTTCATCAAGCAGTACG | |
| | GCGACTGCCTGGGCGACATCGCCGCCAGGGACCTGATCTGCGCCCAGAAG | |
| | TTCAACGGCCTGACCGTGCTGCCCCCCCTGCTGACCGACGAGATGATCGC | |
| | CCAGTACACCAGCGCCCTGCTGGCCGGCACCATCACCAGCGGCTGGACCT | |
| | TCGGCGCCGGCCCCGCCCTGCAGATCCCCTTCCCCATGCAGATGGCCTAC | |
| | AGGTTCAACGGCATCGGCGTGACCCAGAACGTGCTGTACGAGAACCAGAA | |
| | GCTGATCGCCAACCAGTTCAACAGCGCCATCGGCAAGATCCAGGACAGCCT | |
| | GAGCAGCACCCCCAGCGCCCTGGGCAAGCTGCAGGACGTGGTGAACCAGA | |
| | ACGCCCAGGCCCTGAACACCCTGGTGAAGCAGCTGAGCAGCAACTTCGGC | |
| | GCCATCAGCAGCGTGCTGAACGACATCCTGAGCAGGCTGGACCCCCCCGA | |
| | GGCCGAGGTGCAGATCGACAGGCTGATCACCGGCAGGCTGCAGAGCCTGC | |
| | AGACCTACGTGACCCAGCAGCTGATCAGGGCCGCCGAGATCAGGGCCAGC | |
| | GCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGA | |
| | GCGCCCCCCACGGCGTGGTGTTCCTGCACGTGACCTACGTGCCCGCCCAG | |
| | GAGAAGAACTTCACCACCGCCCCCGCCATCTGCCACGACGGCAAGGCCCA | |
| | CTTCCCCAGGGAGGGCGTGTTCGTGAGCAACGGCACCCACTGGTTCGTGA | |
| | CCCAGAGGAACTTCTACGAGCCCCAGATCATCACCACCGACAACACCTTCG | |
| | TGAGCGGCAACTGCGACGTGGTGATCGGCATCGTGAACAACACCGTGTAC | |
| | GACCCCCTGCAGCCCGAGCTGGACAGCTTCAAGGAGGAGCTGGACAAGTA | |
| | CTTCAAGAACCACACCAGCCCCGACGTGGACCTGGGCGACATCAGCGGCA | |
| | TCAACGCCAGCGTGGTGAACATCCAGAAGGAGATCGACAGGCTGAACGAG | |
| | GTGGCCAAGAACCTGAACGAGAGCCTGATCGACCTGCAGGAGCTGGGCAA | |
| | GTACGAGCAGTACATCAAGTGGCCCTGGTACATCTGGCTGGGCTTCATCGC | |
| | CGGCCTGATCGCCATCGTGATGGTGACCATCATGCTGTGCTGCATGACCAG | |
| | CTGCTGCAGCTGCCTGAAGGGCTGCTGCAGCTGCGGCAGCTGCTGCAAGT | |
| | TCGACGAGGACGACAGCGAGCCCGTGCTGAAGGGCGTGAAGCTGCACTAC | |
| | ACCGGAAGCGGAGCCACGAACTTCTCTCTGTTAAAGCAAGCAGGAGATGTT | |
| | GAAGAAAACCCCGGGCCTTTCGGAGCTATTGCTGGTTTCTTGGAAGGAGG | |
| | ATGGGAAGGAATGATTGCAGGTTGGCACGGATACACATCTCATGGAGCAC | |
| | ATGGAGTAGCAGTGGCAGCAGACCTTAAGAGTACCCATGATGA | |
| 6 | ATGTTCGTGTTCCTGGTGCTGCTGCCCCTGGTGAGCAGCCAGTGCGTGAAC | 26 |
| leader | CTGACCACCAGGACCCAGCTGCCCCCCGCCTACACCAACAGCTTCACCAG | |
| sequence, | GGGCGTGTACTACCCCGACAAGGTGTTCAGGAGCAGCGTGCTGCACAGCA | |
| Spike | CCCAGGACCTGTTCCTGCCCTTCTTCAGCAACGTGACCTGGTTCCACGCCA | |
| glyco | TCCACGTGAGCGGCACCAACGGCACCAAGAGGTTCGACAACCCCGTGCTG | |
| protein | CCCTTCAACGACGGCGTGTACTTCGCCAGCACCGAGAAGAGCAACATCATC | |
| (two | AGGGGCTGGATCTTCGGCACCACCCTGGACAGCAAGACCCAGAGCCTGCT | |
| proline | GATCGTGAACAACGCCACCAACGTGGTGATCAAGGTGTGCGAGTTCCAGTT | |
| subst.) | CTGCAACGACCCCTTCCTGGGCGTGTACTACCACAAGAACAACAAGAGCTG | |
| linker, | GATGGAGAGCGAGTTCAGGGTGTACAGCAGCGCCAACAACTGCACCTTCG | |
| HA, Stop | AGTACGTGAGCCAGCCCTTCCTGATGGACCTGGAGGGCAAGCAGGGCAAC | |
| Codon | TTCAAGAACCTGAGGGAGTTCGTGTTCAAGAACATCGACGGCTACTTCAAG | |
| | ATCTACAGCAAGCACACCCCCATCAACCTGGTGAGGGACCTGCCCCAGGG | |
| | CTTCAGCGCCCTGGAGCCCCTGGTGGACCTGCCCATCGGCATCAACATCA | |
| | CCAGGTTCCAGACCCTGCTGGCCCTGCACAGGAGCTACCTGACCCCCGGC | |
| | GACAGCAGCAGCGGCTGGACCGCCGGCGCCGCCGCCTACTACGTGGGCT | |
| | ACCTGCAGCCCAGGACCTTCCTGCTGAAGTACAACGAGAACGGCACCATCA | |
| | CCGACGCCGTGGACTGCGCCCTGGACCCCCTGAGCGAGACCAAGTGCACC | |
| | CTGAAGAGCTTCACCGTGGAGAAGGGCATCTACCAGACCAGCAACTTCAGG | |
| | GTGCAGCCCACCGAGAGCATCGTGAGGTTCCCCAACATCACCAACCTGTGC | |
| | CCCTTCGGCGAGGTGTTCAACGCCACCAGGTTCGCCAGCGTGTACGCCTG | |
| | GAACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCAAG | |
| | CTGAACGACCTGTGCTTCACCAACGTGTACGCCGACAGCTTCGTGATCAGG | |
| | GGCGACGAGGTGAGGCAGATCGCCCCCGGCCAGACCGGCAAGATCGCCG | |
| | ACTACAACTACAAGCTGCCCGACGACTTCACCGGCTGCGTGATCGCCTGGA | |
| | ACAGCAACAACCTGGACAGCAAGGTGGGCGGCAACTACAACTACCTGTACA | |
| | GGCTGTTCAGGAAGAGCAACCTGAAGCCCTTCGAGAGGGACATCAGCACC | |
| | GAGATCTACCAGGCCGGCAGCACCCCCTGCAACGGCGTGGAGGGCTTCAA | |
| | CTGCTACTTCCCCCTGCAGAGCTACGGCTTCCAGCCCACCAACGGCGTGG | |
| | GCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAA | |
| | GTGCGTGAACTTCAACTTCAACGGCCTGACCGGCACCGGCGTGCTGACCG | |
| | AGAGCAACAAGAAGTTCCTGCCCTTCCAGCAGTTCGGCAGGGACATCGCC | |
| | GACACCACCGACGCCGTGAGGGACCCCCAGACCCTGGAGATCCTGGACAT | |
| | CACCCCCTGCAGCTTCGGCGGCGTGAGCGTGATCACCCCCGGCACCAACA | |
| | CCAGCAACCAGGTGGCCGTGCTGTACCAGGACGTGAACTGCACCGAGGTG | |
| | CCCGTGGCCATCCACGCCGACCAGCTGACCCCCACCTGGAGGGTGTACAG | |
| | CACCGGCAGCAACGTGTTCCAGACCAGGGCCGGCTGCCTGATCGGCGCCG | |
| | AGCACGTGAACAACAGCTACGAGTGCGACATCCCCATCGGCGCCGGCATC | |
| | TGCGCCAGCTACCAGACCCAGACCAACAGCCCCAGGAGGGCCAGGAGCGT | |
| | GGCCAGCCAGAGCATCATCGCCTACACCATGAGCCTGGGCGCCGAGAACA | |
| | GCGTGGCCTACAGCAACAACAGCATCGCCATCCCCACCAACTTCACCATCA | |
| | GCGTGACCACCGAGATCCTGCCCGTGAGCATGACCAAGACCAGCGTGGAC | |
| | TGCACCATGTACATCTGCGGCGACAGCACCGAGTGCAGCAACCTGCTGCT | |
| | GCAGTACGGCAGCTTCTGCACCCAGCTGAACAGGGCCCTGACCGGCATCG | |
| | CCGTGGAGCAGGACAAGAACACCCAGGAGGTGTTCGCCCAGGTGAAGCAG | |
| | ATCTACAAGACCCCCCCCATCAAGGACTTCGGCGGCTTCAACTTCAGCCAG | |
| | ATCCTGCCCGACCCCAGCAAGCCCAGCAAGAGGAGCTTCATCGAGGACCT | |
| | GCTGTTCAACAAGGTGACCCTGGCCGACGCCGGCTTCATCAAGCAGTACG | |
| | GCGACTGCCTGGGCGACATCGCCGCCAGGGACCTGATCTGCGCCCAGAAG | |
| | TTCAACGGCCTGACCGTGCTGCCCCCCCTGCTGACCGACGAGATGATCGC | |
| | CCAGTACACCAGCGCCCTGCTGGCCGGCACCATCACCAGCGGCTGGACCT | |
| | TCGGCGCCGGCGCCGCCCTGCAGATCCCCTTCGCCATGCAGATGGCCTAC | |
| | AGGTTCAACGGCATCGGCGTGACCCAGAACGTGCTGTACGAGAACCAGAA | |
| | GCTGATCGCCAACCAGTTCAACAGCGCCATCGGCAAGATCCAGGACAGCCT | |
| | GAGCAGCACCGCCAGCGCCCTGGGCAAGCTGCAGGACGTGGTGAACCAG | |
| | AACGCCCAGGCCCTGAACACCCTGGTGAAGCAGCTGAGCAGCAACTTCGG | |
| | CGCCATCAGCAGCGTGCTGAACGACATCCTGAGCAGGCTGGACCCCCCCG | |
| | AGGCCGAGGTGCAGATCGACAGGCTGATCACCGGCAGGCTGCAGAGCCTG | |
| | CAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAG | |
| | AGCGCCCCCCACGGCGTGGTGTTCCTGCACGTGACCTACGTGCCCGCCCA | |
| | GGAGAAGAACTTCACCACCGCCCCCGCCATCTGCCACGACGGCAAGGCCC | |
| | ACTTCCCCAGGGAGGGCGTGTTCGTGAGCAACGGCACCCACTGGTTCGTG | |
| | ACCCAGAGGAACTTCTACGAGCCCCAGATCATCACCACCGACAACACCTTC | |
| | GTGAGCGGCAACTGCGACGTGGTGATCGGCATCGTGAACAACACCGTGTA | |
| | CGACCCCCTGCAGCCCGAGCTGGACAGCTTCAAGGAGGAGCTGGACAAGT | |
| | ACTTCAAGAACCACACCAGCCCCGACGTGGACCTGGGCGACATCAGCGGC | |
| | ATCAACGCCAGCGTGGTGAACATCCAGAAGGAGATCGACAGGCTGAACGA | |
| | GGTGGCCAAGAACCTGAACGAGAGCCTGATCGACCTGCAGGAGCTGGGCA | |
| | AGTACGAGCAGTACATCAAGTGGCCCTGGTACATCTGGCTGGGCTTCATCG | |
| | CCGGCCTGATCGCCATCGTGATGGTGACCATCATGCTGTGCTGCATGACCA | |
| | GCTGCTGCAGCTGCCTGAAGGGCTGCTGCAGCTGCGGCAGCTGCTGCAAG | |
| | TTCGACGAGGACGACAGCGAGCCCGTGCTGAAGGGCGTGAAGCTGCACTA | |
| | CACCGGAAGCGGAGCCACGAACTTCTCTCTGTTAAAGCAAGCAGGAGATGT | |
| | TGAAGAAAACCCCGGGCCTTTCGGAGCTATTGCTGGTTTCTTGGAAGGAG | |
| | GATGGGAAGGAATGATTGCAGGTTGGCACGGATACACATCTCATGGAGCA | |
| | CATGGAGTAGCAGTGGCAGCAGACCTTAAGAGTACCCATGATGA | |
| Pan-CoV- | TAGTTATTAATAGTAATCAATTACGGGGTCATTAGTTCATAGCCCATATATGG | 27 |
| Flu | AGTTCCGCGTTACATAACTTACGGTAAATGGCCCGCCTGGCTGACCGCCCA | |
| Vaccine | ACGACCCCCGCCCATTGACGTCAATAATGACGTATGTTCCCATAGTAACGC | |
| #7 CoV- | CAATAGGGACTTTCCATTGACGTCAATGGGTGGAGTATTTACGGTAAACTGC | |
| Omicron | CCACTTGGCAGTACATCAAGTGTATCATATGCCAAGTACGCCCCCTATTGAC | |
| BA.2 Spike | GTCAATGACGGTAAATGGCCCGCCTGGCATTATGCCCAGTACATGACCTTA | |
| (HexaPro)- | TGGGACTTTCCTACTTGGCAGTACATCTACGTATTAGTCATCGCTATTACCA | |
| Influenza | TGGTGATGCGGTTTTGGCAGTACATCAATGGGCGTGGATAGCGGTTTGACT | |
| (HA) | CACGGGGATTTCCAAGTCTCCACCCCATTGACGTCAATGGGAGTTTGTTTTG | |
| | GCACCAAAATCAACGGGACTTTCCAAAATGTCGTAACAACTCCGCCCCATTG | |
| | ACGCAAATGGGGGGTAGGCGTGTACGGTGGGAGGTCTATATAAGCAGAGC | |
| | TGGTTTAGTGAACCGTCAGATCGAGAATAAACTAGTATTCTTCTGGTCCCCA | |
| | CAGACTCAGAGAGAACCCGCCACCATGTTCGTGTTCCTGGTGCTGCTGCCC | |
| | CTGGTGAGCAGCCAGTGCGTGAACCTGATCACCAGGACCCAGAGCTACAC | |
| | CAACAGCTTCACCAGGGGCGTGTACTACCCCGACAAGGTGTTCAGGAGCA | |
| | GCGTGCTGCACAGCACCCAGGACCTGTTCCTGCCCTTCTTCAGCAACGTGA | |
| | CCTGGTTCCACGCCATCCACGTGAGCGGCACCAACGGCACCAAGAGGTTC | |
| | GACAACCCCGTGCTGCCCTTCAACGACGGCGTGTACTTCGCCAGCACCGA | |
| | GAAGAGCAACATCATCAGGGGCTGGATCTTCGGCACCACCCTGGACAGCA | |
| | AGACCCAGAGCCTGCTGATCGTGAACAACGCCACCAACGTGGTGATCAAG | |
| | GTGTGCGAGTTCCAGTTCTGCAACGACCCCTTCCTGGACGTGTACTACCAC | |
| | AAGAACAACAAGAGCTGGATGGAGAGCGAGTTCAGGGTGTACAGCAGCGC | |
| | CAACAACTGCACCTTCGAGTACGTGAGCCAGCCCTTCCTGATGGACCTGGA | |
| | GGGCAAGCAGGGCAACTTCAAGAACCTGAGGGAGTTCGTGTTCAAGAACAT | |
| | CGACGGCTACTTCAAGATCTACAGCAAGCACACCCCCATCAACCTGGGCAG | |
| | GGACCTGCCCCAGGGCTTCAGCGCCCTGGAGCCCCTGGTGGACCTGCCCA | |
| | TCGGCATCAACATCACCAGGTTCCAGACCCTGCTGGCCCTGCACAGGAGCT | |
| | ACCTGACCCCCGGCGACAGCAGCAGCGGCTGGACCGCCGGCGCCGCCGC | |
| | CTACTACGTGGGCTACCTGCAGCCCAGGACCTTCCTGCTGAAGTACAACGA | |
| | GAACGGCACCATCACCGACGCCGTGGACTGCGCCCTGGACCCCCTGAGCG | |
| | AGACCAAGTGCACCCTGAAGAGCTTCACCGTGGAGAAGGGCATCTACCAGA | |
| | CCAGCAACTTCAGGGTGCAGCCCACCGAGAGCATCGTGAGGTTCCCCAAC | |
| | ATCACCAACCTGTGCCCCTTCGACGAGGTGTTCAACGCCACCAGGTTCGCC | |
| | AGCGTGTACGCCTGGAACAGGAAGAGGATCAGCAACTGCGTGGCCGACTA | |
| | CAGCGTGCTGTACAACTTCGCCCCCTTCTTCGCCTTCAAGTGCTACGGCGT | |
| | GAGCCCCACCAAGCTGAACGACCTGTGCTTCACCAACGTGTACGCCGACA | |
| | GCTTCGTGATCAGGGGCAACGAGGTGAGCCAGATCGCCCCCGGCCAGACC | |
| | GGCAACATCGCCGACTACAACTACAAGCTGCCCGACGACTTCACCGGCTGC | |
| | GTGATCGCCTGGAACAGCAACAAGCTGGACAGCAAGGTGGGCGGCAACTA | |
| | CAACTACCTGTACAGGCTGTTCAGGAAGAGCAACCTGAAGCCCTTCGAGAG | |
| | GGACATCAGCACCGAGATCTACCAGGCCGGCAACAAGCCCTGCAACGGCG | |
| | TGGCCGGCTTCAACTGCTACTTCCCCCTGAGGAGCTACGGCTTCAGGCCCA | |
| | CCTACGGCGTGGGCCACCAGCCCTACAGGGTGGTGGTGCTGAGCTTCGAG | |
| | CTGCTGCACGCCCCCGCCACCGTGTGCGGCCCCAAGAAGAGCACCAACCT | |
| | GGTGAAGAACAAGTGCGTGAACTTCAACTTCAACGGCCTGACCGGCACCG | |
| | GCGTGCTGACCGAGAGCAACAAGAAGTTCCTGCCCTTCCAGCAGTTCGGCA | |
| | GGGACATCGCCGACACCACCGACGCCGTGAGGGACCCCCAGACCCTGGA | |
| | GATCCTGGACATCACCCCCTGCAGCTTCGGCGGCGTGAGCGTGATCACCC | |
| | CCGGCACCAACACCAGCAACCAGGTGGCCGTGCTGTACCAGGGCGTGAAC | |
| | TGCACCGAGGTGCCCGTGGCCATCCACGCCGACCAGCTGACCCCCACCTG | |
| | GAGGGTGTACAGCACCGGCAGCAACGTGTTCCAGACCAGGGCCGGCTGCC | |
| | TGATCGGCGCCGAGTACGTGAACAACAGCTACGAGTGCGACATCCCCATC | |
| | GGCGCCGGCATCTGCGCCAGCTACCAGACCCAGACCAAGAGCCACCAGCA | |
| | GGCCCAGAGCGTGGCCAGCCAGAGCATCATCGCCTACACCATGAGCCTGG | |
| | GCGCCGAGAACAGCGTGGCCTACAGCAACAACAGCATCGCCATCCCCACC | |
| | AACTTCACCATCAGCGTGACCACCGAGATCCTGCCCGTGAGCATGACCAAG | |
| | ACCAGCGTGGACTGCACCATGTACATCTGCGGCGACAGCACCGAGTGCAG | |
| | CAACCTGCTGCTGCAGTACGGCAGCTTCTGCACCCAGCTGAAGAGGGCCC | |
| | TGACCGGCATCGCCGTGGAGCAGGACAAGAACACCCAGGAGGTGTTCGCC | |
| | CAGGTGAAGCAGATCTACAAGACCCCCCCCATCAAGTACTTCGGCGGCTTC | |
| | AACTTCAGCCAGATCCTGCCCGACCCCAGCAAGCCCAGCAAGAGGAGCCC | |
| | CATCGAGGACCTGCTGTTCAACAAGGTGACCCTGGCCGACGCCGGCTTCAT | |
| | CAAGCAGTACGGCGACTGCCTGGGCGACATCGCCGCCAGGGACCTGATCT | |
| | GCGCCCAGAAGTTCAACGGCCTGACCGTGCTGCCCCCCCTGCTGACCGAC | |
| | GAGATGATCGCCCAGTACACCAGCGCCCTGCTGGCCGGCACCATCACCAG | |
| | CGGCTGGACCTTCGGCGCCGGCCCCGCCCTGCAGATCCCCTTCCCCATGC | |
| | AGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATC | |
| | CAGGACAGCCTGAGCAGCACCCCCAGCGCCCTGGGCAAGCTGCAGGACGT | |
| | GGTGAACCACAACGCCCAGGCCCTGAACACCCTGGTGAAGCAGCTGAGCA | |
| | GCAAGTTCGGCGCCATCAGCAGCGTGCTGAACGACATCCTGAGCAGGCTG | |
| | GACCCCCCCGAGGCCGAGGTGCAGATCGACAGGCTGATCACCGGCAGGCT | |
| | GCAGAGCCTGCAGACCTACGTGACCCAGCAGCTGATCAGGGCCGCCGAGA | |
| | TCAGGGCCAGCGCCAACCTGGCCGCCACCAAGATGAGCGAGTGCGTGCTG | |
| | GGCCAGAGCAAGAGGGTGGACTTCTGCGGCAAGGGCTACCACCTGATGAG | |
| | CTTCCCCCAGAGCGCCCCCCACGGCGTGGTGTTCCTGCACGTGACCTACG | |
| | TGCCCGCCCAGGAGAAGAACTTCACCACCGCCCCCGCCATCTGCCACGAC | |
| | GGCAAGGCCCACTTCCCCAGGGAGGGCGTGTTCGTGAGCAACGGCACCCA | |
| | CTGGTTCGTGACCCAGAGGAACTTCTACGAGCCCCAGATCATCACCACCGA | |
| | CAACACCTTCGTGAGCGGCAACTGCGACGTGGTGATCGGCATCGTGAACAA | |
| | CACCGTGTACGACCCCCTGCAGCCCGAGCTGGACAGCTTCAAGGAGGAGC | |
| | TGGACAAGTACTTCAAGAACCACACCAGCCCCGACGTGGACCTGGGCGAC | |
| | ATCAGCGGCATCAACGCCAGCGTGGTGAACATCCAGAAGGAGATCGACAG | |
| | GCTGAACGAGGTGGCCAAGAACCTGAACGAGAGCCTGATCGACCTGCAGG | |
| | AGCTGGGCAAGTACGAGCAGTACATCAAGTGGCCCTGGTACATCTGGCTG | |
| | GGCTTCATCGCCGGCCTGATCGCCATCGTGATGGTGACCATCATGCTGTGC | |
| | TGCATGACCAGCTGCTGCAGCTGCCTGAAGGGCTGCTGCAGCTGCGGCAG | |
| | CTGCTGCAAGTTCGACGAGGACGACAGCGAGCCCGTGCTGAAGGGCGTGA | |
| | AGCTGCACTACACCGGAAGCGGAGCCACGAACTTCTCTCTGTTAAAGCAAG | |
| | CAGGAGATGTTGAAGAAAACCCCGGGCCTAGCAAAAGCAGGGGATAATTC | |
| | TATTAACCATGAAGACTATCATTGCTTTGAGCTACATTCTATGTCTGGTTTT | |
| | CGCTCAAAAACTTCCTGGAAATGACAATAGCACTGCAACGCTGTGCCTTG | |
| | GGCACCATGCAGTACCAAACGGAACGATAGTGAAAACAATCACGAATGA | |
| | CCAAATTGAAGTTACTAATGCTACTGAGCTGGTTCAGAATTCCTCAATAGG | |
| | TGAAATATGCGACAGTCCTCATCAGATCCTTGATGGAGAAAACTGCACAC | |
| | TAATAGATGCTCTATTGGGAGACCCTCAGTGTGATGGCTTTCAAAATAAGA | |
| | AATGGGACCTTTTTGTTGAACGAAGCAAAGCCTACAGCAACTGTTACCCTT | |
| | ATGATGTGCCGGATTATGCCTCCCTTAGGTCACTAGTTGCCTCATCCGGCA | |
| | CACTGGAGTTTAACAATGAAAGCTTCAATTGGGCTGGAGTCACTCAAAAC | |
| | GGAACAAGTTCTGCTTGCATAAGGGGATCTAATAGTAGTTTCTTTAGTAGA | |
| | TTAAATTGGTTGACCCACTTAAACTTCAAGTACCCAGCATTGAACGTGACT | |
| | ATGCCAAACAATGAACAATTTGACAAATTGTACATTTGGGGGGTTCACCAC | |
| | CCGGGTACGGACAAGGACCAAATCTTCCTGTATGCTCAATCATCAGGAAG | |
| | AATCACAGTATCTACCAAAAGAAGCCAACAAGCTGTAATCCCGAATATCG | |
| | GATCTAGACCCAGAATAAGGAATATCCCTAGCAGAATAAGCATCTATTGG | |
| | ACAATAGTAAAACCGGGAGACATACTTTTGATTAACAGCACAGGGAATCT | |
| | AATTGCTCCTAGGGGTTACTTCAAAATACGAAGTGGGAAAAGCTCAATAA | |
| | TGAGATCAGATGCACCCATTGGCAAATGCAAGTCTGAATGCATCACTCCA | |
| | AATGGAAGCATTCCCAATGACAAACCATTCCAAAATGTAAACAGGATCAC | |
| | ATACGGGGCCTGTCCCAGATATGTTAAGCAAAGCACTCTGAAATTGGCAA | |
| | CAGGAATGCGAAATGTACCAGAGAAACAAACTAGAGGCATATTTGGCGCA | |
| | ATAGCGGGTTTCATAGAAAATGGTTGGGAGGGAATGGTGGATGGTTGGTA | |
| | CGGTTTCAGGCATCAAAATTCTGAGGGAAGAGGACAAGCAGCAGATCTCA | |
| | AAAGCACTCAAGCAGCAATCGATCAAATCAATGGGAAGCTGAATCGATTG | |
| | ATCGGGAAAACCAACGAGAAATTCCATCAGATTGAGAAAGAATTCTCAGA | |
| | AGTAGAAGGGAGAATTCAGGACCTTGAGAAATATGTTGAGGACACAAAAA | |
| | TAGATCTCTGGTCATACAACGCAGAGCTTCTTGTTGCCCTGGAAAACCAAC | |
| | ATACAATTGATCTAACTGACTCAGAAATGAACAAACTGTTTGAAAAAACAA | |
| | AGAAGCAACTGAGGGAAAATGCTGAGGATATGGGCAATGGTTGTTTCAAA | |
| | ATATACCACAAATGTGACAATGCCTGCATAGGATCAATCAGAAATGGAAC | |
| | TTATGACCACGATGTATACAGGGATGAAGCATTAAACAACCGGTTCCAGA | |
| | TCAAGGGAGTTGAGCTGAAGTCAGGGTACAAAGATTGGATCCTATGGATT | |
| | TCCTTTGCCATATCATGTTTTTTGCTTTGTGTTGCTTTGTTGGGGTTCATCAT | |
| | GTGGGCCTGCCAAAAGGGCAACATTAGGTGCAACATTTGCATTTGAGTGC | |
| | ATTAATTAAAAACACCCTTGTTTCTACTGGAAGCGGAGCCACGAACTTCTC | |
| | TCTGTTAAAGCAAGCAGGAGATGTTGAAGAAAACCCCGGGCCTATTTTCTA | |
| | ATATCCACAAAATGAAGGCAATAATTGTACTACTCATGGTAGTAACATCCA | |
| | ATGCAGATCGAATCTGCACTGGGATAACATCGTCAAACTCACCACATGTC | |
| | GTCAAAACTGCTACTCAAGGGGAGGTCAACGTGACCGGTGTAATACCACT | |
| | GACAACAACACCCACCAAATCTCATTTTGCAAATCTCAAAGGAACAGAAA | |
| | CCAGGGGGAAACTATGCCCAAAATGCCTCAACTGCACAGATCTGGATGTA | |
| | GCCTTGGGCAGACCAAAATGCACAGGGAAAATACCCTCTGCAAGGGTTTC | |
| | AATACTCCATGAAGTCAGACCTGTTACATCTGGGTGCTTTCCTATAATGCA | |
| | CGATAGAACAAAAATTAGACAGCTGCCTAACCTTCTCCGAGGATACGAAC | |
| | ATGTCAGGTTATCAACTCACAACGTTATCAATGCAAAAGATGCACCAGGA | |
| | AGACCCTACGAAATTGGAACCTCAGGGTCTTGCCCTAACATTACCAATGG | |
| | AAACGGATTCTTCGCAACAATGGCTTGGGCCGTCCCAAAAAACAAAACAG | |
| | CAACAAATCCATTAACAATAGAAGTACCATACATTTGTACAGAAGGAGAA | |
| | GACCAAATTACCGTTTGGGGGTTCCACTCTGACAACGAGACCCAAATGGC | |
| | AAAGCTCTATGGGGACTCAAAGCCCCAGAAGTTCACCTCATCTGCCAACG | |
| | GAGTGACCACACATTACGTTTCACAGATTGGTGGCTTCCCAAATCAAACA | |
| | GAAGACGGAGGACTACCACAAAGTGGCAGAATTGTTGTTGATTACATGGT | |
| | GCAGAAATCTGGAAAAACAGGAACAATTACCTATCAAAGAGGTATTTTATT | |
| | GCCTCAAAAAGTGTGGTGCGCAAGTGGCAGGAGCAAGGTAATAAAAGGA | |
| | TCCTTGCCCTTAATTGGAGAAGCAGATTGCCTCCATGAAAAATACGGTGG | |
| | ATTAAACAAAAGCAAGCCTTACTACACAGGGGAACATGCAAAGGCCATAG | |
| | GAAATTGCCCAATATGGGTGAAAACACCCTTGAAGCTGGCCAATGGAACC | |
| | AAATATAGACCCCCTGCAAAACTATTAAAGGAAAGAGGTTTCTTCGGAGC | |
| | CATTGCTGGTTTCTTAGAGGGAGGATGGGAAGGAATGATTGCAGGTTGGC | |
| | ACGGATACACATCCCATGGGGCACATGGAGTAGCGGTGGCAGCTGACCT | |
| | TAAGAGCACTCAAGAGGCCATAAACAAGATAACAAAAAATCTCAACTCTT | |
| | TGAGTGAGCTGGAAGTAAAGAATCTTCAAAGACTAAGCGGTGCCATGGAT | |
| | GAACTCCACAACGAAATACTAGAACTAGATGAGAAAGTGGATGATCTCAG | |
| | AGCTGACACAATAAGCTCACAAATAGAACTCGCAGTCCTGCTTTCCAATG | |
| | AAGGAATAATAAACAGTGAAGATGAACATCTCTTGGCGCTTGAAAGAAAG | |
| | CTGAAGAAAATGCTGGGCCCCTCTGCTGTAGAGATAGGGAATGGATGCTT | |
| | TGAAACCAAACACAAGTGCAACCAGACCTGCCTCGACAGAATAGCTGCTG | |
| | GTACCTTTGATGCAGGAGAATTTTCTCTCCCCACCTTTGATTCACTGAATAT | |
| | TACTGCTGCATCTTTAAATGACGACGGATTGGACAATCATACTATACTGCT | |
| | TTACTACTCAACTGCTGCCTCCAGTTTGGCTGTAACACTGATGATAGCTAT | |
| | CTTTGTTGTTTATATGGTCTCCAGAGACAATGTTTCTTGCTCCATTTGTCTA | |
| | TAAGGAAAGTTAAGCCCTGTATTTTCCTTTATTGTAGTGCTTGTTTGCTTGT | |
| | TGTCATTACAAAGAAACGTTATTGAAAAATGGAAGCGGAGCCACGAACTTC | |
| | TCTCTGTTAAAGCAAGCAGGAGATGTTGAAGAAAACCCCGGGCCTTTCGGA | |
| | GCTATTGCTGGTTTCTTGGAAGGAGGATGGGAAGGAATGATTGCAGGTTGG | |
| | CACGGATACACATCTCATGGAGCACATGGAGTAGCAGTGGCAGCAGACCTT | |
| | AAGAGTACCCATGATGACTCGAGCTGGTACTGCATGCACGCAATGCTAGCT | |
| | GCCCCTTTCCCGTCCTGGGTACCCCGAGTCTCCCCCGACCTCGGGTCCCA | |
| | GGTATGCTCCCACCTCCACCTGCCCCACTCACCACCTCTGCTAGTTCCAGA | |
| | CACCTCCCAAGCACGCAGCAATGCAGCTCAAAACGCTTAGCCTAGCCACAC | |
| | CCCCACGGGAAACAGCAGTGATTAACCTTTAGCAATAAACGAAAGTTTAACT | |
| | AAGCTATACTAACCCCAGGGTTGGTCAATTTCGTGCCAGCCACACCCTGGA | |
| | GCTAGCAAAAAAAA | |
| Pan-CoV- | TAGTTATTAATAGTAATCAATTACGGGGTCATTAGTTCATAGCCCATATATGG | 28 |
| Flu | AGTTCCGCGTTACATAACTTACGGTAAATGGCCCGCCTGGCTGACCGCCCA | |
| Vaccine | ACGACCCCCGCCCATTGACGTCAATAATGACGTATGTTCCCATAGTAACGC | |
| #8 CoV- | CAATAGGGACTTTCCATTGACGTCAATGGGTGGAGTATTTACGGTAAACTGC | |
| Omicron | CCACTTGGCAGTACATCAAGTGTATCATATGCCAAGTACGCCCCCTATTGAC | |
| BA.2 Spike | GTCAATGACGGTAAATGGCCCGCCTGGCATTATGCCCAGTACATGACCTTA | |
| (2 proline | TGGGACTTTCCTACTTGGCAGTACATCTACGTATTAGTCATCGCTATTACCA | |
| subst.)- | TGGTGATGCGGTTTTGGCAGTACATCAATGGGCGTGGATAGCGGTTTGACT | |
| Influenza | CACGGGGATTTCCAAGTCTCCACCCCATTGACGTCAATGGGAGTTTGTTTTG | |
| (HA) | GCACCAAAATCAACGGGACTTTCCAAAATGTCGTAACAACTCCGCCCCATTG | |
| | ACGCAAATGGGGGGTAGGCGTGTACGGTGGGAGGTCTATATAAGCAGAGC | |
| | TGGTTTAGTGAACCGTCAGATCGAGAATAAACTAGTATTCTTCTGGTCCCCA | |
| | CAGACTCAGAGAGAACCCGCCACCATGTTCGTGTTCCTGGTGCTGCTGCCC | |
| | CTGGTGAGCAGCCAGTGCGTGAACCTGATCACCAGGACCCAGAGCTACAC | |
| | CAACAGCTTCACCAGGGGCGTGTACTACCCCGACAAGGTGTTCAGGAGCA | |
| | GCGTGCTGCACAGCACCCAGGACCTGTTCCTGCCCTTCTTCAGCAACGTGA | |
| | CCTGGTTCCACGCCATCCACGTGAGCGGCACCAACGGCACCAAGAGGTTC | |
| | GACAACCCCGTGCTGCCCTTCAACGACGGCGTGTACTTCGCCAGCACCGA | |
| | GAAGAGCAACATCATCAGGGGCTGGATCTTCGGCACCACCCTGGACAGCA | |
| | AGACCCAGAGCCTGCTGATCGTGAACAACGCCACCAACGTGGTGATCAAG | |
| | GTGTGCGAGTTCCAGTTCTGCAACGACCCCTTCCTGGACGTGTACTACCAC | |
| | AAGAACAACAAGAGCTGGATGGAGAGCGAGTTCAGGGTGTACAGCAGCGC | |
| | CAACAACTGCACCTTCGAGTACGTGAGCCAGCCCTTCCTGATGGACCTGGA | |
| | GGGCAAGCAGGGCAACTTCAAGAACCTGAGGGAGTTCGTGTTCAAGAACAT | |
| | CGACGGCTACTTCAAGATCTACAGCAAGCACACCCCCATCAACCTGGGCAG | |
| | GGACCTGCCCCAGGGCTTCAGCGCCCTGGAGCCCCTGGTGGACCTGCCCA | |
| | TCGGCATCAACATCACCAGGTTCCAGACCCTGCTGGCCCTGCACAGGAGCT | |
| | ACCTGACCCCCGGCGACAGCAGCAGCGGCTGGACCGCCGGCGCCGCCGC | |
| | CTACTACGTGGGCTACCTGCAGCCCAGGACCTTCCTGCTGAAGTACAACGA | |
| | GAACGGCACCATCACCGACGCCGTGGACTGCGCCCTGGACCCCCTGAGCG | |
| | AGACCAAGTGCACCCTGAAGAGCTTCACCGTGGAGAAGGGCATCTACCAGA | |
| | CCAGCAACTTCAGGGTGCAGCCCACCGAGAGCATCGTGAGGTTCCCCAAC | |
| | ATCACCAACCTGTGCCCCTTCGACGAGGTGTTCAACGCCACCAGGTTCGCC | |
| | AGCGTGTACGCCTGGAACAGGAAGAGGATCAGCAACTGCGTGGCCGACTA | |
| | CAGCGTGCTGTACAACTTCGCCCCCTTCTTCGCCTTCAAGTGCTACGGCGT | |
| | GAGCCCCACCAAGCTGAACGACCTGTGCTTCACCAACGTGTACGCCGACA | |
| | GCTTCGTGATCAGGGGCAACGAGGTGAGCCAGATCGCCCCCGGCCAGACC | |
| | GGCAACATCGCCGACTACAACTACAAGCTGCCCGACGACTTCACCGGCTGC | |
| | GTGATCGCCTGGAACAGCAACAAGCTGGACAGCAAGGTGGGCGGCAACTA | |
| | CAACTACCTGTACAGGCTGTTCAGGAAGAGCAACCTGAAGCCCTTCGAGAG | |
| | GGACATCAGCACCGAGATCTACCAGGCCGGCAACAAGCCCTGCAACGGCG | |
| | TGGCCGGCTTCAACTGCTACTTCCCCCTGAGGAGCTACGGCTTCAGGCCCA | |
| | CCTACGGCGTGGGCCACCAGCCCTACAGGGTGGTGGTGCTGAGCTTCGAG | |
| | CTGCTGCACGCCCCCGCCACCGTGTGCGGCCCCAAGAAGAGCACCAACCT | |
| | GGTGAAGAACAAGTGCGTGAACTTCAACTTCAACGGCCTGACCGGCACCG | |
| | GCGTGCTGACCGAGAGCAACAAGAAGTTCCTGCCCTTCCAGCAGTTCGGCA | |
| | GGGACATCGCCGACACCACCGACGCCGTGAGGGACCCCCAGACCCTGGA | |
| | GATCCTGGACATCACCCCCTGCAGCTTCGGCGGCGTGAGCGTGATCACCC | |
| | CCGGCACCAACACCAGCAACCAGGTGGCCGTGCTGTACCAGGGCGTGAAC | |
| | TGCACCGAGGTGCCCGTGGCCATCCACGCCGACCAGCTGACCCCCACCTG | |
| | GAGGGTGTACAGCACCGGCAGCAACGTGTTCCAGACCAGGGCCGGCTGCC | |
| | TGATCGGCGCCGAGTACGTGAACAACAGCTACGAGTGCGACATCCCCATC | |
| | GGCGCCGGCATCTGCGCCAGCTACCAGACCCAGACCAAGAGCCACCAGCA | |
| | GGCCCAGAGCGTGGCCAGCCAGAGCATCATCGCCTACACCATGAGCCTGG | |
| | GCGCCGAGAACAGCGTGGCCTACAGCAACAACAGCATCGCCATCCCCACC | |
| | AACTTCACCATCAGCGTGACCACCGAGATCCTGCCCGTGAGCATGACCAAG | |
| | ACCAGCGTGGACTGCACCATGTACATCTGCGGCGACAGCACCGAGTGCAG | |
| | CAACCTGCTGCTGCAGTACGGCAGCTTCTGCACCCAGCTGAAGAGGGCCC | |
| | TGACCGGCATCGCCGTGGAGCAGGACAAGAACACCCAGGAGGTGTTCGCC | |
| | CAGGTGAAGCAGATCTACAAGACCCCCCCCATCAAGTACTTCGGCGGCTTC | |
| | AACTTCAGCCAGATCCTGCCCGACCCCAGCAAGCCCAGCAAGAGGAGCTT | |
| | CATCGAGGACCTGCTGTTCAACAAGGTGACCCTGGCCGACGCCGGCTTCAT | |
| | CAAGCAGTACGGCGACTGCCTGGGCGACATCGCCGCCAGGGACCTGATCT | |
| | GCGCCCAGAAGTTCAACGGCCTGACCGTGCTGCCCCCCCTGCTGACCGAC | |
| | GAGATGATCGCCCAGTACACCAGCGCCCTGCTGGCCGGCACCATCACCAG | |
| | CGGCTGGACCTTCGGCGCCGGCGCCGCCCTGCAGATCCCCTTCGCCATGC | |
| | AGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATC | |
| | CAGGACAGCCTGAGCAGCACCGCCAGCGCCCTGGGCAAGCTGCAGGACG | |
| | TGGTGAACCACAACGCCCAGGCCCTGAACACCCTGGTGAAGCAGCTGAGC | |
| | AGCAAGTTCGGCGCCATCAGCAGCGTGCTGAACGACATCCTGAGCAGGCT | |
| | GGACCCCCCCGAGGCCGAGGTGCAGATCGACAGGCTGATCACCGGCAGG | |
| | CTGCAGAGCCTGCAGACCTACGTGACCCAGCAGCTGATCAGGGCCGCCGA | |
| | GATCAGGGCCAGCGCCAACCTGGCCGCCACCAAGATGAGCGAGTGCGTGC | |
| | TGGGCCAGAGCAAGAGGGTGGACTTCTGCGGCAAGGGCTACCACCTGATG | |
| | AGCTTCCCCCAGAGCGCCCCCCACGGCGTGGTGTTCCTGCACGTGACCTA | |
| | CGTGCCCGCCCAGGAGAAGAACTTCACCACCGCCCCCGCCATCTGCCACG | |
| | ACGGCAAGGCCCACTTCCCCAGGGAGGGCGTGTTCGTGAGCAACGGCACC | |
| | CACTGGTTCGTGACCCAGAGGAACTTCTACGAGCCCCAGATCATCACCACC | |
| | GACAACACCTTCGTGAGCGGCAACTGCGACGTGGTGATCGGCATCGTGAA | |
| | CAACACCGTGTACGACCCCCTGCAGCCCGAGCTGGACAGCTTCAAGGAGG | |
| | AGCTGGACAAGTACTTCAAGAACCACACCAGCCCCGACGTGGACCTGGGC | |
| | GACATCAGCGGCATCAACGCCAGCGTGGTGAACATCCAGAAGGAGATCGA | |
| | CAGGCTGAACGAGGTGGCCAAGAACCTGAACGAGAGCCTGATCGACCTGC | |
| | AGGAGCTGGGCAAGTACGAGCAGTACATCAAGTGGCCCTGGTACATCTGG | |
| | CTGGGCTTCATCGCCGGCCTGATCGCCATCGTGATGGTGACCATCATGCTG | |
| | TGCTGCATGACCAGCTGCTGCAGCTGCCTGAAGGGCTGCTGCAGCTGCGG | |
| | CAGCTGCTGCAAGTTCGACGAGGACGACAGCGAGCCCGTGCTGAAGGGCG | |
| | TGAAGCTGCACTACACCGGAAGCGGAGCCACGAACTTCTCTCTGTTAAAGC | |
| | AAGCAGGAGATGTTGAAGAAAACCCCGGGCCTATGAAGGCAATACTAGTAG | |
| | TTCTGCTATATACATTTGCAACCGCAAATGCAGACACATTATGTATAGGTTAT | |
| | CATGCGAACAACTCAACAGACACTGTAGACACAGTACTAGAAAAGAATGTAA | |
| | CAGTAACACACTCTGTTAACCTTCTAGAAGACAAGCATAACGGGAAACTATG | |
| | CAAACTAAGAGGGGTAGCCCCATTGCATTTGGGTAAATGTAACATTGCTGG | |
| | CTGGATCCTGGGAAATCCAGAGTGTGAATCACTCTCCACAGCAAGCTCATG | |
| | GTCCTACATTGTGGAAACATCTAGTTCAGACAATGGAACGTGTTACCCAGGA | |
| | GATTTCATCGATTATGAGGAGCTAAGAGAGCAATTGAGCTCAGTGTCATCAT | |
| | TTGAAAGGTTTGAGATATTCCCCAAGACAAGTTCATGGCCCAATCATGACTC | |
| | GAACAAAGGTGTAACGACAGCATGTCCTCATGCTGGAGCAAAAAGCTTCTA | |
| | CAAAAATTTAATATGGCTAGTTAAAAAAGGAAATTCATACCCAAAGCTCAGCA | |
| | AATCCTACATTAATGATAAAGGGAAAGAAGTCCTCGTGCTATGGGGCATTCA | |
| | CCATCCACCTACTAGTGCTGACCAACAAAGTCTCTATCAGAATGCAGATGCA | |
| | TATGTTTTTGTGGGGACATCAAGATACAGCAAGAAGTTCAAGCCGGAAATAG | |
| | CAATAAGGCCCAAAGTGAGGGATCAAGAAGGGAGAATGAACTATTACTGGA | |
| | CACTAGTAGAGCCGGGAGACAAAATAACATTCGAAGCAACTGGAAATCTAG | |
| | TGGTACCGAGATATGCATTCGCAATGGAAAGAAATGCTGGATCTGGTATTAT | |
| | CATTTCAGATACACCAGTCCACGATTGCAATACAACTTGTCAGACACCCAAG | |
| | GGTGCTATAAACACCAGCCTCCCATTTCAGAACATACATCCGATCACAATTG | |
| | GAAAATGTCCAAAATATGTAAAAAGCACAAAATTGAGACTGGCCACAGGATT | |
| | GAGGAATGTCCCGTCCATTCAATCTAGAGGCCTATTTGGGGCCATTGCCGG | |
| | TTTCATTGAAGGGGGGTGGACAGGGATGGTAGATGGATGGTACGGTTATCA | |
| | CCATCAAAATGAGCAGGGGTCAGGATATGCAGCCGACCTGAAGAGCACACA | |
| | GAATGCCATTGACGAGATTACTAACAAAGTAAACTCTGTTATTGAAAAAATGA | |
| | ATACACAGTTCACAGCAGTAGGTAAAGAGTTCAACCACCTGGAAAAAAGAAT | |
| | AGAGAATTTAAATAAAAAAGTTGATGATGGTTTCCTGGACATTTGGACTTACA | |
| | ATGCCGAACTGTTGGTTCTATTGGAAAATGAAAGAACTTTGGACTACCACGA | |
| | TTCAAAGGTGAAGAACTTATATGAAAAGGTAAGAAGCCAGTTAAAAAACAAT | |
| | GCCAAGGAAATTGGAAACGGCTGCTTTGAATTTTACCACAAATGTGATAACA | |
| | CGTGCATGGAAAGTGTCAAAAATGGGACTTATGACTACCCAAAATACTCAGA | |
| | GGAAGCAAAATTAAACAGAGAAGAAATAGATGGGGTAAAGCTGGAATCAAC | |
| | AAGGATTTACCAGATTTTGGCGATCTATTCAACCGTCGCCAGTTCATTGGTA | |
| | CTGGTAGTCTCCCTGGGGGCAATCAGTTTCTGGATGTGCTCTAATGGGTCT | |
| | CTACAGTGTAGAATATGTATTTAAGGAAGCGGAGCCACGAACTTCTCTCTGT | |
| | TAAAGCAAGCAGGAGATGTTGAAGAAAACCCCGGGCCTAGCAAAAGCAGG | |
| | GGATAATTCTATTAACCATGAAGACTATCATTGCTTTGAGCTACATTCTATGT | |
| | CTGGTTTTCGCTCAAAAACTTCCTGGAAATGACAATAGCACTGCAACGCTGT | |
| | GCCTTGGGCACCATGCAGTACCAAACGGAACGATAGTGAAAACAATCACGA | |
| | ATGACCAAATTGAAGTTACTAATGCTACTGAGCTGGTTCAGAATTCCTCAAT | |
| | AGGTGAAATATGCGACAGTCCTCATCAGATCCTTGATGGAGAAAACTGCAC | |
| | ACTAATAGATGCTCTATTGGGAGACCCTCAGTGTGATGGCTTTCAAAATAAG | |
| | AAATGGGACCTTTTTGTTGAACGAAGCAAAGCCTACAGCAACTGTTACCCTT | |
| | ATGATGTGCCGGATTATGCCTCCCTTAGGTCACTAGTTGCCTCATCCGGCA | |
| | CACTGGAGTTTAACAATGAAAGCTTCAATTGGGCTGGAGTCACTCAAAACG | |
| | GAACAAGTTCTGCTTGCATAAGGGGATCTAATAGTAGTTTCTTTAGTAGATTA | |
| | AATTGGTTGACCCACTTAAACTTCAAGTACCCAGCATTGAACGTGACTATGC | |
| | CAAACAATGAACAATTTGACAAATTGTACATTTGGGGGGTTCACCACCCGGG | |
| | TACGGACAAGGACCAAATCTTCCTGTATGCTCAATCATCAGGAAGAATCACA | |
| | GTATCTACCAAAAGAAGCCAACAAGCTGTAATCCCGAATATCGGATCTAGAC | |
| | CCAGAATAAGGAATATCCCTAGCAGAATAAGCATCTATTGGACAATAGTAAA | |
| | ACCGGGAGACATACTTTTGATTAACAGCACAGGGAATCTAATTGCTCCTAGG | |
| | GGTTACTTCAAAATACGAAGTGGGAAAAGCTCAATAATGAGATCAGATGCAC | |
| | CCATTGGCAAATGCAAGTCTGAATGCATCACTCCAAATGGAAGCATTCCCAA | |
| | TGACAAACCATTCCAAAATGTAAACAGGATCACATACGGGGCCTGTCCCAG | |
| | ATATGTTAAGCAAAGCACTCTGAAATTGGCAACAGGAATGCGAAATGTACCA | |
| | GAGAAACAAACTAGAGGCATATTTGGCGCAATAGCGGGTTTCATAGAAAAT | |
| | GGTTGGGAGGGAATGGTGGATGGTTGGTACGGTTTCAGGCATCAAAATTCT | |
| | GAGGGAAGAGGACAAGCAGCAGATCTCAAAAGCACTCAAGCAGCAATCGAT | |
| | CAAATCAATGGGAAGCTGAATCGATTGATCGGGAAAACCAACGAGAAATTC | |
| | CATCAGATTGAGAAAGAATTCTCAGAAGTAGAAGGGAGAATTCAGGACCTT | |
| | GAGAAATATGTTGAGGACACAAAAATAGATCTCTGGTCATACAACGCAGAGC | |
| | TTCTTGTTGCCCTGGAAAACCAACATACAATTGATCTAACTGACTCAGAAAT | |
| | GAACAAACTGTTTGAAAAAAAAAGAAGCAACTGAGGGAAAATGCTGAGGA | |
| | TATGGGCAATGGTTGTTTCAAAATATACCACAAATGTGACAATGCCTGCATA | |
| | GGATCAATCAGAAATGGAACTTATGACCACGATGTATACAGGGATGAAGCAT | |
| | TAAACAACCGGTTCCAGATCAAGGGAGTTGAGCTGAAGTCAGGGTACAAAG | |
| | ATTGGATCCTATGGATTTCCTTTGCCATATCATGTTTTTTGCTTTGTGTTGCT | |
| | TTGTTGGGGTTCATCATGTGGGCCTGCCAAAAGGGCAACATTAGGTGCAAC | |
| | ATTTGCATTTGAGTGCATTAATTAAAAACACCCTTGTTTCTACTGGAAGCGGA | |
| | GCCACGAACTTCTCTCTGTTAAAGCAAGCAGGAGATGTTGAAGAAAACCCC | |
| | GGGCCTATTTTCTAATATCCACAAAATGAAGGCAATAATTGTACTACTCATG | |
| | GTAGTAACATCCAATGCAGATCGAATCTGCACTGGGATAACATCGTCAAACT | |
| | CACCACATGTCGTCAAAACTGCTACTCAAGGGGAGGTCAACGTGACCGGTG | |
| | TAATACCACTGACAACAACACCCACCAAATCTCATTTTGCAAATCTCAAAGG | |
| | AACAGAAACCAGGGGGAAACTATGCCCAAAATGCCTCAACTGCACAGATCT | |
| | GGATGTAGCCTTGGGCAGACCAAAATGCACAGGGAAAATACCCTCTGCAAG | |
| | GGTTTCAATACTCCATGAAGTCAGACCTGTTACATCTGGGTGCTTTCCTATA | |
| | ATGCACGATAGAACAAAAATTAGACAGCTGCCTAACCTTCTCCGAGGATACG | |
| | AACATGTCAGGTTATCAACTCACAACGTTATCAATGCAAAAGATGCACCAGG | |
| | AAGACCCTACGAAATTGGAACCTCAGGGTCTTGCCCTAACATTACCAATGGA | |
| | AACGGATTCTTCGCAACAATGGCTTGGGCCGTCCCAAAAAACAAAACAGCA | |
| | ACAAATCCATTAACAATAGAAGTACCATACATTTGTACAGAAGGAGAAGACC | |
| | AAATTACCGTTTGGGGGTTCCACTCTGACAACGAGACCCAAATGGCAAAGC | |
| | TCTATGGGGACTCAAAGCCCCAGAAGTTCACCTCATCTGCCAACGGAGTGA | |
| | CCACACATTACGTTTCACAGATTGGTGGCTTCCCAAATCAAACAGAAGACGG | |
| | AGGACTACCACAAAGTGGCAGAATTGTTGTTGATTACATGGTGCAGAAATCT | |
| | GGAAAAACAGGAACAATTACCTATCAAAGAGGTATTTTATTGCCTCAAAAAG | |
| | TGTGGTGCGCAAGTGGCAGGAGCAAGGTAATAAAAGGATCCTTGCCCTTAA | |
| | TTGGAGAAGCAGATTGCCTCCATGAAAAATACGGTGGATTAAACAAAAGCAA | |
| | GCCTTACTACACAGGGGAACATGCAAAGGCCATAGGAAATTGCCCAATATG | |
| | GGTGAAAACACCCTTGAAGCTGGCCAATGGAACCAAATATAGACCCCCTGC | |
| | AAAACTATTAAAGGAAAGAGGTTTCTTCGGAGCCATTGCTGGTTTCTTAGAG | |
| | GGAGGATGGGAAGGAATGATTGCAGGTTGGCACGGATACACATCCCATGG | |
| | GGCACATGGAGTAGCGGTGGCAGCTGACCTTAAGAGCACTCAAGAGGCCA | |
| | TAAACAAGATAACAAAAAATCTCAACTCTTTGAGTGAGCTGGAAGTAAAGAA | |
| | TCTTCAAAGACTAAGCGGTGCCATGGATGAACTCCACAACGAAATACTAGAA | |
| | CTAGATGAGAAAGTGGATGATCTCAGAGCTGACACAATAAGCTCACAAATAG | |
| | AACTCGCAGTCCTGCTTTCCAATGAAGGAATAATAAACAGTGAAGATGAACA | |
| | TCTCTTGGCGCTTGAAAGAAAGCTGAAGAAAATGCTGGGCCCCTCTGCTGT | |
| | AGAGATAGGGAATGGATGCTTTGAAACCAAACACAAGTGCAACCAGACCTG | |
| | CCTCGACAGAATAGCTGCTGGTACCTTTGATGCAGGAGAATTTTCTCTCCCC | |
| | ACCTTTGATTCACTGAATATTACTGCTGCATCTTTAAATGACGACGGATTGG | |
| | ACAATCATACTATACTGCTTTACTACTCAACTGCTGCCTCCAGTTTGGCTGTA | |
| | ACACTGATGATAGCTATCTTTGTTGTTTATATGGTCTCCAGAGACAATGTTTC | |
| | TTGCTCCATTTGTCTATAAGGAAAGTTAAGCCCTGTATTTTCCTTTATTGTAG | |
| | TGCTTGTTTGCTTGTTGTCATTACAAAGAAACGTTATTGAAAAATGGAAGCG | |
| | GAGCCACGAACTTCTCTCTGTTAAAGCAAGCAGGAGATGTTGAAGAAAACC | |
| | CCGGGCCTTTCGGAGCTATTGCTGGTTTCTTGGAAGGAGGATGGGAAGGAA | |
| | TGATTGCAGGTTGGCACGGATACACATCTCATGGAGCACATGGAGTAGCAG | |
| | TGGCAGCAGACCTTAAGAGTACCCATGATGACTCGAGCTGGTACTGCATGC | |
| | ACGCAATGCTAGCTGCCCCTTTCCCGTCCTGGGTACCCCGAGTCTCCCCCG | |
| | ACCTCGGGTCCCAGGTATGCTCCCACCTCCACCTGCCCCACTCACCACCTC | |
| | TGCTAGTTCCAGACACCTCCCAAGCACGCAGCAATGCAGCTCAAAACGCTT | |
| | AGCCTAGCCACACCCCCACGGGAAACAGCAGTGATTAACCTTTAGCAATAA | |
| | ACGAAAGTTTAACTAAGCTATACTAACCCCAGGGTTGGTCAATTTCGTGCCA | |
| | GCCACACCCTGGAGCTAGCAAAAAAAA | |
| Pan-CoV- | CTCGACATTGATTATTGACTAGTTATTAATAGTAATCAATTACGGGGTCATTA | 29 |
| Flu | GTTCATAGCCCATATATGGAGTTCCGCGTTACATAACTTACGGTAAATGGCC | |
| Vaccine | CGCCTGGCTGACCGCCCAACGACCCCCGCCCATTGACGTCAATAATGACGT | |
| #9 CoV- | ATGTTCCCATAGTAACGCCAATAGGGACTTTCCATTGACGTCAATGGGTGGA | |
| Omicron | GTATTTACGGTAAACTGCCCACTTGGCAGTACATCAAGTGTATCATATGCCA | |
| BA.2 Spike | AGTACGCCCCCTATTGACGTCAATGACGGTAAATGGCCCGCCTGGCATTAT | |
| (HexaPro)- | GCCCAGTACATGACCTTATGGGACTTTCCTACTTGGCAGTACATCTACGTAT | |
| Influenza | TAGTCATCGCTATTACCATGGTCGAGGTGAGCCCCACGTTCTGCTTCACTCT | |
| (HA) | CCCCATCTCCCCCCCCTCCCCACCCCCAATTTTGTATTTATTTATTTTTTAAT | |
| | TATTTTGTGCAGCGATGGGGGCGGGGGGGGGGGGGGGGCGGCGCGCCAG | |
| | GCGGGGCGGGGCGGGGCGAGGGGCGGGGCGGGGCGAGGCGGAGAGGT | |
| | GCGGCGGCAGCCAATCAGAGCGGCGCGCTCCGAAAGTTTCCTTTTATGGC | |
| | GAGGCGGCGGCGGCGGCGGCCCTATAAAAAGCGAAGCGCGCGGCGGGC | |
| | GGGAGTCGCTGCGCGCTGCCTTCGCCCCGTGCCCCGCTCCGCCGCCGCC | |
| | TCGCGCCGCCCGCCCCGGCTCTGACTGACCGCGTTACTCCCACAGGTGAG | |
| | CGGGCGGGACGGCCCTTCTCCTCCGGGCTGTAATTAGCGCTTGGTTTAATG | |
| | ACGGCTTGTTTCTTTTCTGTGGCTGCGTGAAAGCCTTGAGGGGCTCCGGGA | |
| | GGGCCCTTTGTGCGGGGGGAGCGGCTCGGGGGGTGCGTGCGTGTGTGTG | |
| | TGCGTGGGGAGCGCCGCGTGCGGCTCCGCGCTGCCCGGCGGCTGTGAGC | |
| | GCTGCGGGCGCGGCGCGGGGCTTTGTGCGCTCCGCAGTGTGCGCGAGGG | |
| | GAGCGCGGCCGGGGGCGGTGCCCCGCGGTGCGGGGGGGGCTGCGAGGG | |
| | GAACAAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCCCGAGT | |
| | TGCTGAGCACGGCCCGGCTTCGGGTGCGGGGCTCCGTACGGGGCGTGGC | |
| | GCGGGGCTCGCCGTGCCGGGGGGGGGGGGCGGCAGGTGGGGGTGCCG | |
| | GGCGGGGGGGGGCCGCCTCGGGCCGGGGAGGGCTCGGGGGAGGGGCG | |
| | CGGCGGCCCCCGGAGCGCCGGCGGCTGTCGAGGCGCGGCGAGCCGCAG | |
| | CCATTGCCTTTTATGGTAATCGTGCGAGAGGGCGCAGGGACTTCCTTTGTC | |
| | CCAAATCTGTGCGGAGCCGAAATCTGGGAGGCGCCGCCGCACCCCCTCTA | |
| | GCGGGCGCGGGGCGAAGCGGTGCGGCGCCGGCAGGAAGGAAATGGGCG | |
| | GGGAGGGCCTTCGTGCGTCGCCGCGCCGCCGTCCCCTTCTCCCTCTCCAG | |
| | CCTCGGGGCTGTCCGCGGGGGGACGGCTGCCTTCGGGGGGGACGGGGC | |
| | AGGGGGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGTGCTG | |
| | GTTATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTTCCT | |
| | GGTGCTGCTGCCCCTGGTGAGCAGCCAGTGCGTGAACCTGATCACCAGGA | |
| | CCCAGAGCTACACCAACAGCTTCACCAGGGGCGTGTACTACCCCGACAAG | |
| | GTGTTCAGGAGCAGCGTGCTGCACAGCACCCAGGACCTGTTCCTGCCCTTC | |
| | TTCAGCAACGTGACCTGGTTCCACGCCATCCACGTGAGCGGCACCAACGG | |
| | CACCAAGAGGTTCGACAACCCCGTGCTGCCCTTCAACGACGGCGTGTACTT | |
| | CGCCAGCACCGAGAAGAGCAACATCATCAGGGGCTGGATCTTCGGCACCA | |
| | CCCTGGACAGCAAGACCCAGAGCCTGCTGATCGTGAACAACGCCACCAAC | |
| | GTGGTGATCAAGGTGTGCGAGTTCCAGTTCTGCAACGACCCCTTCCTGGAC | |
| | GTGTACTACCACAAGAACAACAAGAGCTGGATGGAGAGCGAGTTCAGGGTG | |
| | TACAGCAGCGCCAACAACTGCACCTTCGAGTACGTGAGCCAGCCCTTCCTG | |
| | ATGGACCTGGAGGGCAAGCAGGGCAACTTCAAGAACCTGAGGGAGTTCGT | |
| | GTTCAAGAACATCGACGGCTACTTCAAGATCTACAGCAAGCACACCCCCAT | |
| | CAACCTGGGCAGGGACCTGCCCCAGGGCTTCAGCGCCCTGGAGCCCCTG | |
| | GTGGACCTGCCCATCGGCATCAACATCACCAGGTTCCAGACCCTGCTGGCC | |
| | CTGCACAGGAGCTACCTGACCCCCGGCGACAGCAGCAGCGGCTGGACCG | |
| | CCGGCGCCGCCGCCTACTACGTGGGCTACCTGCAGCCCAGGACCTTCCTG | |
| | CTGAAGTACAACGAGAACGGCACCATCACCGACGCCGTGGACTGCGCCCT | |
| | GGACCCCCTGAGCGAGACCAAGTGCACCCTGAAGAGCTTCACCGTGGAGA | |
| | AGGGCATCTACCAGACCAGCAACTTCAGGGTGCAGCCCACCGAGAGCATC | |
| | GTGAGGTTCCCCAACATCACCAACCTGTGCCCCTTCGACGAGGTGTTCAAC | |
| | GCCACCAGGTTCGCCAGCGTGTACGCCTGGAACAGGAAGAGGATCAGCAA | |
| | CTGCGTGGCCGACTACAGCGTGCTGTACAACTTCGCCCCCTTCTTCGCCTT | |
| | CAAGTGCTACGGCGTGAGCCCCACCAAGCTGAACGACCTGTGCTTCACCAA | |
| | CGTGTACGCCGACAGCTTCGTGATCAGGGGCAACGAGGTGAGCCAGATCG | |
| | CCCCCGGCCAGACCGGCAACATCGCCGACTACAACTACAAGCTGCCCGAC | |
| | GACTTCACCGGCTGCGTGATCGCCTGGAACAGCAACAAGCTGGACAGCAA | |
| | GGTGGGCGGCAACTACAACTACCTGTACAGGCTGTTCAGGAAGAGCAACCT | |
| | GAAGCCCTTCGAGAGGGACATCAGCACCGAGATCTACCAGGCCGGCAACA | |
| | AGCCCTGCAACGGCGTGGCCGGCTTCAACTGCTACTTCCCCCTGAGGAGC | |
| | TACGGCTTCAGGCCCACCTACGGCGTGGGCCACCAGCCCTACAGGGTGGT | |
| | GGTGCTGAGCTTCGAGCTGCTGCACGCCCCCGCCACCGTGTGCGGCCCCA | |
| | AGAAGAGCACCAACCTGGTGAAGAACAAGTGCGTGAACTTCAACTTCAACG | |
| | GCCTGACCGGCACCGGCGTGCTGACCGAGAGCAACAAGAAGTTCCTGCCC | |
| | TTCCAGCAGTTCGGCAGGGACATCGCCGACACCACCGACGCCGTGAGGGA | |
| | CCCCCAGACCCTGGAGATCCTGGACATCACCCCCTGCAGCTTCGGCGGCG | |
| | TGAGCGTGATCACCCCCGGCACCAACACCAGCAACCAGGTGGCCGTGCTG | |
| | TACCAGGGCGTGAACTGCACCGAGGTGCCCGTGGCCATCCACGCCGACCA | |
| | GCTGACCCCCACCTGGAGGGTGTACAGCACCGGCAGCAACGTGTTCCAGA | |
| | CCAGGGCCGGCTGCCTGATCGGCGCCGAGTACGTGAACAACAGCTACGAG | |
| | TGCGACATCCCCATCGGCGCCGGCATCTGCGCCAGCTACCAGACCCAGAC | |
| | CAAGAGCCACCAGCAGGCCCAGAGCGTGGCCAGCCAGAGCATCATCGCCT | |
| | ACACCATGAGCCTGGGCGCCGAGAACAGCGTGGCCTACAGCAACAACAGC | |
| | ATCGCCATCCCCACCAACTTCACCATCAGCGTGACCACCGAGATCCTGCCC | |
| | GTGAGCATGACCAAGACCAGCGTGGACTGCACCATGTACATCTGCGGCGA | |
| | CAGCACCGAGTGCAGCAACCTGCTGCTGCAGTACGGCAGCTTCTGCACCC | |
| | AGCTGAAGAGGGCCCTGACCGGCATCGCCGTGGAGCAGGACAAGAACACC | |
| | CAGGAGGTGTTCGCCCAGGTGAAGCAGATCTACAAGACCCCCCCCATCAA | |
| | GTACTTCGGCGGCTTCAACTTCAGCCAGATCCTGCCCGACCCCAGCAAGCC | |
| | CAGCAAGAGGAGCCCCATCGAGGACCTGCTGTTCAACAAGGTGACCCTGG | |
| | CCGACGCCGGCTTCATCAAGCAGTACGGCGACTGCCTGGGCGACATCGCC | |
| | GCCAGGGACCTGATCTGCGCCCAGAAGTTCAACGGCCTGACCGTGCTGCC | |
| | CCCCCTGCTGACCGACGAGATGATCGCCCAGTACACCAGCGCCCTGCTGG | |
| | CCGGCACCATCACCAGCGGCTGGACCTTCGGCGCCGGCCCCGCCCTGCA | |
| | GATCCCCTTCCCCATGCAGATGGCCTACAGGTTCAACGGCATCGGCGTGAC | |
| | CCAGAACGTGCTGTACGAGAACCAGAAGCTGATCGCCAACCAGTTCAACAG | |
| | CGCCATCGGCAAGATCCAGGACAGCCTGAGCAGCACCCCCAGCGCCCTGG | |
| | GCAAGCTGCAGGACGTGGTGAACCACAACGCCCAGGCCCTGAACACCCTG | |
| | GTGAAGCAGCTGAGCAGCAAGTTCGGCGCCATCAGCAGCGTGCTGAACGA | |
| | CATCCTGAGCAGGCTGGACCCCCCCGAGGCCGAGGTGCAGATCGACAGGC | |
| | TGATCACCGGCAGGCTGCAGAGCCTGCAGACCTACGTGACCCAGCAGCTG | |
| | ATCAGGGCCGCCGAGATCAGGGCCAGCGCCAACCTGGCCGCCACCAAGAT | |
| | GAGCGAGTGCGTGCTGGGCCAGAGCAAGAGGGTGGACTTCTGCGGCAAG | |
| | GGCTACCACCTGATGAGCTTCCCCCAGAGCGCCCCCCACGGCGTGGTGTT | |
| | CCTGCACGTGACCTACGTGCCCGCCCAGGAGAAGAACTTCACCACCGCCC | |
| | CCGCCATCTGCCACGACGGCAAGGCCCACTTCCCCAGGGAGGGCGTGTTC | |
| | GTGAGCAACGGCACCCACTGGTTCGTGACCCAGAGGAACTTCTACGAGCC | |
| | CCAGATCATCACCACCGACAACACCTTCGTGAGCGGCAACTGCGACGTGGT | |
| | GATCGGCATCGTGAACAACACCGTGTACGACCCCCTGCAGCCCGAGCTGG | |
| | ACAGCTTCAAGGAGGAGCTGGACAAGTACTTCAAGAACCACACCAGCCCCG | |
| | ACGTGGACCTGGGCGACATCAGCGGCATCAACGCCAGCGTGGTGAACATC | |
| | CAGAAGGAGATCGACAGGCTGAACGAGGTGGCCAAGAACCTGAACGAGAG | |
| | CCTGATCGACCTGCAGGAGCTGGGCAAGTACGAGCAGTACATCAAGTGGC | |
| | CCTGGTACATCTGGCTGGGCTTCATCGCCGGCCTGATCGCCATCGTGATGG | |
| | TGACCATCATGCTGTGCTGCATGACCAGCTGCTGCAGCTGCCTGAAGGGCT | |
| | GCTGCAGCTGCGGCAGCTGCTGCAAGTTCGACGAGGACGACAGCGAGCCC | |
| | GTGCTGAAGGGCGTGAAGCTGCACTACACCGGAAGCGGAGCCACGAACTT | |
| | CTCTCTGTTAAAGCAAGCAGGAGATGTTGAAGAAAACCCCGGGCCTATGAA | |
| | GGCAATACTAGTAGTTCTGCTATATACATTTGCAACCGCAAATGCAGACACA | |
| | TTATGTATAGGTTATCATGCGAACAACTCAACAGACACTGTAGACACAGTAC | |
| | TAGAAAAGAATGTAACAGTAACACACTCTGTTAACCTTCTAGAAGACAAGCA | |
| | TAACGGGAAACTATGCAAACTAAGAGGGGTAGCCCCATTGCATTTGGGTAA | |
| | ATGTAACATTGCTGGCTGGATCCTGGGAAATCCAGAGTGTGAATCACTCTC | |
| | CACAGCAAGCTCATGGTCCTACATTGTGGAAACATCTAGTTCAGACAATGGA | |
| | ACGTGTTACCCAGGAGATTTCATCGATTATGAGGAGCTAAGAGAGCAATTGA | |
| | GCTCAGTGTCATCATTTGAAAGGTTTGAGATATTCCCCAAGACAAGTTCATG | |
| | GCCCAATCATGACTCGAACAAAGGTGTAACGACAGCATGTCCTCATGCTGG | |
| | AGCAAAAAGCTTCTACAAAAATTTAATATGGCTAGTTAAAAAAGGAAATTCAT | |
| | ACCCAAAGCTCAGCAAATCCTACATTAATGATAAAGGGAAAGAAGTCCTCGT | |
| | GCTATGGGGCATTCACCATCCACCTACTAGTGCTGACCAACAAAGTCTCTAT | |
| | CAGAATGCAGATGCATATGTTTTTGTGGGGACATCAAGATACAGCAAGAAGT | |
| | TCAAGCCGGAAATAGCAATAAGGCCCAAAGTGAGGGATCAAGAAGGGAGAA | |
| | TGAACTATTACTGGACACTAGTAGAGCCGGGAGACAAAATAACATTCGAAG | |
| | CAACTGGAAATCTAGTGGTACCGAGATATGCATTCGCAATGGAAAGAAATG | |
| | CTGGATCTGGTATTATCATTTCAGATACACCAGTCCACGATTGCAATACAAC | |
| | TTGTCAGACACCCAAGGGTGCTATAAACACCAGCCTCCCATTTCAGAACATA | |
| | CATCCGATCACAATTGGAAAATGTCCAAAATATGTAAAAAGCACAAAATTGA | |
| | GACTGGCCACAGGATTGAGGAATGTCCCGTCCATTCAATCTAGAGGCCTAT | |
| | TTGGGGCCATTGCCGGTTTCATTGAAGGGGGGGGACAGGGATGGTAGAT | |
| | GGATGGTACGGTTATCACCATCAAAATGAGCAGGGGTCAGGATATGCAGCC | |
| | GACCTGAAGAGCACACAGAATGCCATTGACGAGATTACTAACAAAGTAAACT | |
| | CTGTTATTGAAAAAATGAATACACAGTTCACAGCAGTAGGTAAAGAGTTCAA | |
| | CCACCTGGAAAAAAGAATAGAGAATTTAAATAAAAAAGTTGATGATGGTTTC | |
| | CTGGACATTTGGACTTACAATGCCGAACTGTTGGTTCTATTGGAAAATGAAA | |
| | GAACTTTGGACTACCACGATTCAAAGGTGAAGAACTTATATGAAAAGGTAAG | |
| | AAGCCAGTTAAAAAACAATGCCAAGGAAATTGGAAACGGCTGCTTTGAATTT | |
| | TACCACAAATGTGATAACACGTGCATGGAAAGTGTCAAAAATGGGACTTATG | |
| | ACTACCCAAAATACTCAGAGGAAGCAAAATTAAACAGAGAAGAAATAGATGG | |
| | GGTAAAGCTGGAATCAACAAGGATTTACCAGATTTTGGCGATCTATTCAACC | |
| | GTCGCCAGTTCATTGGTACTGGTAGTCTCCCTGGGGGCAATCAGTTTCTGG | |
| | ATGTGCTCTAATGGGTCTCTACAGTGTAGAATATGTATTTAAGGAAGCGGAG | |
| | CCACGAACTTCTCTCTGTTAAAGCAAGCAGGAGATGTTGAAGAAAACCCCG | |
| | GGCCTAGCAAAAGCAGGGGATAATTCTATTAACCATGAAGACTATCATTGCT | |
| | TTGAGCTACATTCTATGTCTGGTTTTCGCTCAAAAACTTCCTGGAAATGACAA | |
| | TAGCACTGCAACGCTGTGCCTTGGGCACCATGCAGTACCAAACGGAACGAT | |
| | AGTGAAAACAATCACGAATGACCAAATTGAAGTTACTAATGCTACTGAGCTG | |
| | GTTCAGAATTCCTCAATAGGTGAAATATGCGACAGTCCTCATCAGATCCTTG | |
| | ATGGAGAAAACTGCACACTAATAGATGCTCTATTGGGAGACCCTCAGTGTG | |
| | ATGGCTTTCAAAATAAGAAATGGGACCTTTTTGTTGAACGAAGCAAAGCCTA | |
| | CAGCAACTGTTACCCTTATGATGTGCCGGATTATGCCTCCCTTAGGTCACTA | |
| | GTTGCCTCATCCGGCACACTGGAGTTTAACAATGAAAGCTTCAATTGGGCT | |
| | GGAGTCACTCAAAACGGAACAAGTTCTGCTTGCATAAGGGGATCTAATAGTA | |
| | GTTTCTTTAGTAGATTAAATTGGTTGACCCACTTAAACTTCAAGTACCCAGCA | |
| | TTGAACGTGACTATGCCAAACAATGAACAATTTGACAAATTGTACATTTGGG | |
| | GGGTTCACCACCCGGGTACGGACAAGGACCAAATCTTCCTGTATGCTCAAT | |
| | CATCAGGAAGAATCACAGTATCTACCAAAAGAAGCCAACAAGCTGTAATCCC | |
| | GAATATCGGATCTAGACCCAGAATAAGGAATATCCCTAGCAGAATAAGCATC | |
| | TATTGGACAATAGTAAAACCGGGAGACATACTTTTGATTAACAGCACAGGGA | |
| | ATCTAATTGCTCCTAGGGGTTACTTCAAAATACGAAGTGGGAAAAGCTCAAT | |
| | AATGAGATCAGATGCACCCATTGGCAAATGCAAGTCTGAATGCATCACTCCA | |
| | AATGGAAGCATTCCCAATGACAAACCATTCCAAAATGTAAACAGGATCACAT | |
| | ACGGGGCCTGTCCCAGATATGTTAAGCAAAGCACTCTGAAATTGGCAACAG | |
| | GAATGCGAAATGTACCAGAGAAACAAACTAGAGGCATATTTGGCGCAATAG | |
| | CGGGTTTCATAGAAAATGGTTGGGAGGGAATGGTGGATGGTTGGTACGGTT | |
| | TCAGGCATCAAAATTCTGAGGGAAGAGGACAAGCAGCAGATCTCAAAAGCA | |
| | CTCAAGCAGCAATCGATCAAATCAATGGGAAGCTGAATCGATTGATCGGGA | |
| | AAACCAACGAGAAATTCCATCAGATTGAGAAAGAATTCTCAGAAGTAGAAGG | |
| | GAGAATTCAGGACCTTGAGAAATATGTTGAGGACACAAAAATAGATCTCTGG | |
| | TCATACAACGCAGAGCTTCTTGTTGCCCTGGAAAACCAACATACAATTGATC | |
| | TAACTGACTCAGAAATGAACAAACTGTTTGAAAAAACAAAGAAGCAACTGAG | |
| | GGAAAATGCTGAGGATATGGGCAATGGTTGTTTCAAAATATACCACAAATGT | |
| | GACAATGCCTGCATAGGATCAATCAGAAATGGAACTTATGACCACGATGTAT | |
| | ACAGGGATGAAGCATTAAACAACCGGTTCCAGATCAAGGGAGTTGAGCTGA | |
| | AGTCAGGGTACAAAGATTGGATCCTATGGATTTCCTTTGCCATATCATGTTTT | |
| | TTGCTTTGTGTTGCTTTGTTGGGGTTCATCATGTGGGCCTGCCAAAAGGGC | |
| | AACATTAGGTGCAACATTTGCATTTGAGTGCATTAATTAAAAACACCCTTGTT | |
| | TCTACTGGAAGCGGAGCCACGAACTTCTCTCTGTTAAAGCAAGCAGGAGAT | |
| | GTTGAAGAAAACCCCGGGCCTATTTTCTAATATCCACAAAATGAAGGCAATA | |
| | ATTGTACTACTCATGGTAGTAACATCCAATGCAGATCGAATCTGCACTGGGA | |
| | TAACATCGTCAAACTCACCACATGTCGTCAAAACTGCTACTCAAGGGGAGGT | |
| | CAACGTGACCGGTGTAATACCACTGACAACAACACCCACCAAATCTCATTTT | |
| | GCAAATCTCAAAGGAACAGAAACCAGGGGGAAACTATGCCCAAAATGCCTC | |
| | AACTGCACAGATCTGGATGTAGCCTTGGGCAGACCAAAATGCACAGGGAAA | |
| | ATACCCTCTGCAAGGGTTTCAATACTCCATGAAGTCAGACCTGTTACATCTG | |
| | GGTGCTTTCCTATAATGCACGATAGAACAAAAATTAGACAGCTGCCTAACCT | |
| | TCTCCGAGGATACGAACATGTCAGGTTATCAACTCACAACGTTATCAATGCA | |
| | AAAGATGCACCAGGAAGACCCTACGAAATTGGAACCTCAGGGTCTTGCCCT | |
| | AACATTACCAATGGAAACGGATTCTTCGCAACAATGGCTTGGGCCGTCCCA | |
| | AAAAACAAAACAGCAACAAATCCATTAACAATAGAAGTACCATACATTTGTAC | |
| | AGAAGGAGAAGACCAAATTACCGTTTGGGGGTTCCACTCTGACAACGAGAC | |
| | CCAAATGGCAAAGCTCTATGGGGACTCAAAGCCCCAGAAGTTCACCTCATC | |
| | TGCCAACGGAGTGACCACACATTACGTTTCACAGATTGGTGGCTTCCCAAAT | |
| | CAAACAGAAGACGGAGGACTACCACAAAGTGGCAGAATTGTTGTTGATTAC | |
| | ATGGTGCAGAAATCTGGAAAAACAGGAACAATTACCTATCAAAGAGGTATTT | |
| | TATTGCCTCAAAAAGTGTGGTGCGCAAGTGGCAGGAGCAAGGTAATAAAAG | |
| | GATCCTTGCCCTTAATTGGAGAAGCAGATTGCCTCCATGAAAAATACGGTG | |
| | GATTAAACAAAAGCAAGCCTTACTACACAGGGGAACATGCAAAGGCCATAG | |
| | GAAATTGCCCAATATGGGTGAAAACACCCTTGAAGCTGGCCAATGGAACCA | |
| | AATATAGACCCCCTGCAAAACTATTAAAGGAAAGAGGTTTCTTCGGAGCCAT | |
| | TGCTGGTTTCTTAGAGGGAGGATGGGAAGGAATGATTGCAGGTTGGCACG | |
| | GATACACATCCCATGGGGCACATGGAGTAGCGGTGGCAGCTGACCTTAAGA | |
| | GCACTCAAGAGGCCATAAACAAGATAACAAAAAATCTCAACTCTTTGAGTGA | |
| | GCTGGAAGTAAAGAATCTTCAAAGACTAAGCGGTGCCATGGATGAACTCCA | |
| | CAACGAAATACTAGAACTAGATGAGAAAGTGGATGATCTCAGAGCTGACAC | |
| | AATAAGCTCACAAATAGAACTCGCAGTCCTGCTTTCCAATGAAGGAATAATA | |
| | AACAGTGAAGATGAACATCTCTTGGCGCTTGAAAGAAAGCTGAAGAAAATG | |
| | CTGGGCCCCTCTGCTGTAGAGATAGGGAATGGATGCTTTGAAACCAAACAC | |
| | AAGTGCAACCAGACCTGCCTCGACAGAATAGCTGCTGGTACCTTTGATGCA | |
| | GGAGAATTTTCTCTCCCCACCTTTGATTCACTGAATATTACTGCTGCATCTTT | |
| | AAATGACGACGGATTGGACAATCATACTATACTGCTTTACTACTCAACTGCT | |
| | GCCTCCAGTTTGGCTGTAACACTGATGATAGCTATCTTTGTTGTTTATATGGT | |
| | CTCCAGAGACAATGTTTCTTGCTCCATTTGTCTATAAGGAAAGTTAAGCCCT | |
| | GTATTTTCCTTTATTGTAGTGCTTGTTTGCTTGTTGTCATTACAAAGAAACGT | |
| | TATTGAAAAATGGAAGCGGAGCCACGAACTTCTCTCTGTTAAAGCAAGCAG | |
| | GAGATGTTGAAGAAAACCCCGGGCCTTTCGGAGCTATTGCTGGTTTCTTGG | |
| | AAGGAGGATGGGAAGGAATGATTGCAGGTTGGCACGGATACACATCTCATG | |
| | GAGCACATGGAGTAGCAGTGGCAGCAGACCTTAAGAGTACCCATGATGACT | |
| | CGAGCTGGTACTGCATGCACGCAATGCTAGCTGCCCCTTTCCCGTCCTGGG | |
| | TACCCCGAGTCTCCCCCGACCTCGGGTCCCAGGTATGCTCCCACCTCCACC | |
| | TGCCCCACTCACCACCTCTGCTAGTTCCAGACACCTCCCAAGCACGCAGCA | |
| | ATGCAGCTCAAAACGCTTAGCCTAGCCACACCCCCACGGGAAACAGCAGTG | |
| | ATTAACCTTTAGCAATAAACGAAAGTTTAACTAAGCTATACTAACCCCAGGGT | |
| | TGGTCAATTTCGTGCCAGCCACACCCTGGAGCTAGCAAAAAAAA | |
| Pan-CoV- | CTCGACATTGATTATTGACTAGTTATTAATAGTAATCAATTACGGGGTCATTA | 30 |
| Flu | GTTCATAGCCCATATATGGAGTTCCGCGTTACATAACTTACGGTAAATGGCC | |
| Vaccine | CGCCTGGCTGACCGCCCAACGACCCCCGCCCATTGACGTCAATAATGACGT | |
| #10 CoV- | ATGTTCCCATAGTAACGCCAATAGGGACTTTCCATTGACGTCAATGGGTGGA | |
| Omicron | GTATTTACGGTAAACTGCCCACTTGGCAGTACATCAAGTGTATCATATGCCA | |
| BA.2 Spike | AGTACGCCCCCTATTGACGTCAATGACGGTAAATGGCCCGCCTGGCATTAT | |
| (2 proline | GCCCAGTACATGACCTTATGGGACTTTCCTACTTGGCAGTACATCTACGTAT | |
| subst.)- | TAGTCATCGCTATTACCATGGTCGAGGTGAGCCCCACGTTCTGCTTCACTCT | |
| Influenza | CCCCATCTCCCCCCCCTCCCCACCCCCAATTTTGTATTTATTTATTTTTTAAT | |
| (HA) | TATTTTGTGCAGCGATGGGGGCGGGGGGGGGGGGGGGGCGCGCGCCAG | |
| | GCGGGGCGGGGCGGGGCGAGGGGCGGGGCGGGGCGAGGCGGAGAGGT | |
| | GCGGCGGCAGCCAATCAGAGCGGCGCGCTCCGAAAGTTTCCTTTTATGGC | |
| | GAGGCGGCGGCGGCGGCGGCCCTATAAAAAGCGAAGCGCGCGGCGGGC | |
| | GGGAGTCGCTGCGCGCTGCCTTCGCCCCGTGCCCCGCTCCGCCGCCGCC | |
| | TCGCGCCGCCCGCCCCGGCTCTGACTGACCGCGTTACTCCCACAGGTGAG | |
| | CGGGCGGGACGGCCCTTCTCCTCCGGGCTGTAATTAGCGCTTGGTTTAATG | |
| | ACGGCTTGTTTCTTTTCTGTGGCTGCGTGAAAGCCTTGAGGGGCTCCGGGA | |
| | GGGCCCTTTGTGCGGGGGGAGCGGCTCGGGGGGTGCGTGCGTGTGTGTG | |
| | TGCGTGGGGAGCGCCGCGTGCGGCTCCGCGCTGCCCGGCGGCTGTGAGC | |
| | GCTGCGGGCGCGGCGCGGGGCTTTGTGCGCTCCGCAGTGTGCGCGAGGG | |
| | GAGCGCGGCCGGGGGGGGTGCCCCGCGGTGCGGGGGGGGCTGCGAGGG | |
| | GAACAAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCCCGAGT | |
| | TGCTGAGCACGGCCCGGCTTCGGGTGCGGGGCTCCGTACGGGGCGTGGC | |
| | GCGGGGCTCGCCGTGCCGGGGGGGGGGGGCGGCAGGTGGGGGTGCCG | |
| | GGCGGGGCGGGGCCGCCTCGGGCCGGGGAGGGCTCGGGGGAGGGGCG | |
| | CGGCGGCCCCCGGAGCGCCGGCGGCTGTCGAGGCGCGGCGAGCCGCAG | |
| | CCATTGCCTTTTATGGTAATCGTGCGAGAGGGCGCAGGGACTTCCTTTGTC | |
| | CCAAATCTGTGCGGAGCCGAAATCTGGGAGGCGCCGCCGCACCCCCTCTA | |
| | GCGGGCGCGGGGCGAAGCGGTGCGGCGCCGGCAGGAAGGAAATGGGCG | |
| | GGGAGGGCCTTCGTGCGTCGCCGCGCCGCCGTCCCCTTCTCCCTCTCCAG | |
| | CCTCGGGGCTGTCCGCGGGGGGACGGCTGCCTTCGGGGGGGACGGGGC | |
| | AGGGGGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGTGCTG | |
| | GTTATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTTCCT | |
| | GGTGCTGCTGCCCCTGGTGAGCAGCCAGTGCGTGAACCTGATCACCAGGA | |
| | CCCAGAGCTACACCAACAGCTTCACCAGGGGCGTGTACTACCCCGACAAG | |
| | GTGTTCAGGAGCAGCGTGCTGCACAGCACCCAGGACCTGTTCCTGCCCTTC | |
| | TTCAGCAACGTGACCTGGTTCCACGCCATCCACGTGAGCGGCACCAACGG | |
| | CACCAAGAGGTTCGACAACCCCGTGCTGCCCTTCAACGACGGCGTGTACTT | |
| | CGCCAGCACCGAGAAGAGCAACATCATCAGGGGCTGGATCTTCGGCACCA | |
| | CCCTGGACAGCAAGACCCAGAGCCTGCTGATCGTGAACAACGCCACCAAC | |
| | GTGGTGATCAAGGTGTGCGAGTTCCAGTTCTGCAACGACCCCTTCCTGGAC | |
| | GTGTACTACCACAAGAACAACAAGAGCTGGATGGAGAGCGAGTTCAGGGTG | |
| | TACAGCAGCGCCAACAACTGCACCTTCGAGTACGTGAGCCAGCCCTTCCTG | |
| | ATGGACCTGGAGGGCAAGCAGGGCAACTTCAAGAACCTGAGGGAGTTCGT | |
| | GTTCAAGAACATCGACGGCTACTTCAAGATCTACAGCAAGCACACCCCCAT | |
| | CAACCTGGGCAGGGACCTGCCCCAGGGCTTCAGCGCCCTGGAGCCCCTG | |
| | GTGGACCTGCCCATCGGCATCAACATCACCAGGTTCCAGACCCTGCTGGCC | |
| | CTGCACAGGAGCTACCTGACCCCCGGCGACAGCAGCAGCGGCTGGACCG | |
| | CCGGCGCCGCCGCCTACTACGTGGGCTACCTGCAGCCCAGGACCTTCCTG | |
| | CTGAAGTACAACGAGAACGGCACCATCACCGACGCCGTGGACTGCGCCCT | |
| | GGACCCCCTGAGCGAGACCAAGTGCACCCTGAAGAGCTTCACCGTGGAGA | |
| | AGGGCATCTACCAGACCAGCAACTTCAGGGTGCAGCCCACCGAGAGCATC | |
| | GTGAGGTTCCCCAACATCACCAACCTGTGCCCCTTCGACGAGGTGTTCAAC | |
| | GCCACCAGGTTCGCCAGCGTGTACGCCTGGAACAGGAAGAGGATCAGCAA | |
| | CTGCGTGGCCGACTACAGCGTGCTGTACAACTTCGCCCCCTTCTTCGCCTT | |
| | CAAGTGCTACGGCGTGAGCCCCACCAAGCTGAACGACCTGTGCTTCACCAA | |
| | CGTGTACGCCGACAGCTTCGTGATCAGGGGCAACGAGGTGAGCCAGATCG | |
| | CCCCCGGCCAGACCGGCAACATCGCCGACTACAACTACAAGCTGCCCGAC | |
| | GACTTCACCGGCTGCGTGATCGCCTGGAACAGCAACAAGCTGGACAGCAA | |
| | GGTGGGCGGCAACTACAACTACCTGTACAGGCTGTTCAGGAAGAGCAACCT | |
| | GAAGCCCTTCGAGAGGGACATCAGCACCGAGATCTACCAGGCCGGCAACA | |
| | AGCCCTGCAACGGCGTGGCCGGCTTCAACTGCTACTTCCCCCTGAGGAGC | |
| | TACGGCTTCAGGCCCACCTACGGCGTGGGCCACCAGCCCTACAGGGTGGT | |
| | GGTGCTGAGCTTCGAGCTGCTGCACGCCCCCGCCACCGTGTGCGGCCCCA | |
| | AGAAGAGCACCAACCTGGTGAAGAACAAGTGCGTGAACTTCAACTTCAACG | |
| | GCCTGACCGGCACCGGCGTGCTGACCGAGAGCAACAAGAAGTTCCTGCCC | |
| | TTCCAGCAGTTCGGCAGGGACATCGCCGACACCACCGACGCCGTGAGGGA | |
| | CCCCCAGACCCTGGAGATCCTGGACATCACCCCCTGCAGCTTCGGCGGCG | |
| | TGAGCGTGATCACCCCCGGCACCAACACCAGCAACCAGGTGGCCGTGCTG | |
| | TACCAGGGCGTGAACTGCACCGAGGTGCCCGTGGCCATCCACGCCGACCA | |
| | GCTGACCCCCACCTGGAGGGTGTACAGCACCGGCAGCAACGTGTTCCAGA | |
| | CCAGGGCCGGCTGCCTGATCGGCGCCGAGTACGTGAACAACAGCTACGAG | |
| | TGCGACATCCCCATCGGCGCCGGCATCTGCGCCAGCTACCAGACCCAGAC | |
| | CAAGAGCCACCAGCAGGCCCAGAGCGTGGCCAGCCAGAGCATCATCGCCT | |
| | ACACCATGAGCCTGGGCGCCGAGAACAGCGTGGCCTACAGCAACAACAGC | |
| | ATCGCCATCCCCACCAACTTCACCATCAGCGTGACCACCGAGATCCTGCCC | |
| | GTGAGCATGACCAAGACCAGCGTGGACTGCACCATGTACATCTGCGGCGA | |
| | CAGCACCGAGTGCAGCAACCTGCTGCTGCAGTACGGCAGCTTCTGCACCC | |
| | AGCTGAAGAGGGCCCTGACCGGCATCGCCGTGGAGCAGGACAAGAACACC | |
| | CAGGAGGTGTTCGCCCAGGTGAAGCAGATCTACAAGACCCCCCCCATCAA | |
| | GTACTTCGGCGGCTTCAACTTCAGCCAGATCCTGCCCGACCCCAGCAAGCC | |
| | CAGCAAGAGGAGCTTCATCGAGGACCTGCTGTTCAACAAGGTGACCCTGGC | |
| | CGACGCCGGCTTCATCAAGCAGTACGGCGACTGCCTGGGCGACATCGCCG | |
| | CCAGGGACCTGATCTGCGCCCAGAAGTTCAACGGCCTGACCGTGCTGCCC | |
| | CCCCTGCTGACCGACGAGATGATCGCCCAGTACACCAGCGCCCTGCTGGC | |
| | CGGCACCATCACCAGCGGCTGGACCTTCGGCGCCGGCGCCGCCCTGCAG | |
| | ATCCCCTTCGCCATGCAGATGGCCTACAGGTTCAACGGCATCGGCGTGACC | |
| | CAGAACGTGCTGTACGAGAACCAGAAGCTGATCGCCAACCAGTTCAACAGC | |
| | GCCATCGGCAAGATCCAGGACAGCCTGAGCAGCACCGCCAGCGCCCTGGG | |
| | CAAGCTGCAGGACGTGGTGAACCACAACGCCCAGGCCCTGAACACCCTGG | |
| | TGAAGCAGCTGAGCAGCAAGTTCGGCGCCATCAGCAGCGTGCTGAACGAC | |
| | ATCCTGAGCAGGCTGGACCCCCCCGAGGCCGAGGTGCAGATCGACAGGCT | |
| | GATCACCGGCAGGCTGCAGAGCCTGCAGACCTACGTGACCCAGCAGCTGA | |
| | TCAGGGCCGCCGAGATCAGGGCCAGCGCCAACCTGGCCGCCACCAAGATG | |
| | AGCGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTGTTC | |
| | CTGCACGTGACCTACGTGCCCGCCCAGGAGAAGAACTTCACCACCGCCCC | |
| | CGCCATCTGCCACGACGGCAAGGCCCACTTCCCCAGGGAGGGCGTGTTCG | |
| | TGAGCAACGGCACCCACTGGTTCGTGACCCAGAGGAACTTCTACGAGCCC | |
| | CAGATCATCACCACCGACAACACCTTCGTGAGCGGCAACTGCGACGTGGTG | |
| | ATCGGCATCGTGAACAACACCGTGTACGACCCCCTGCAGCCCGAGCTGGA | |
| | CAGCTTCAAGGAGGAGCTGGACAAGTACTTCAAGAACCACACCAGCCCCGA | |
| | CGTGGACCTGGGCGACATCAGCGGCATCAACGCCAGCGTGGTGAACATCC | |
| | AGAAGGAGATCGACAGGCTGAACGAGGTGGCCAAGAACCTGAACGAGAGC | |
| | CTGATCGACCTGCAGGAGCTGGGCAAGTACGAGCAGTACATCAAGTGGCC | |
| | CTGGTACATCTGGCTGGGCTTCATCGCCGGCCTGATCGCCATCGTGATGGT | |
| | GACCATCATGCTGTGCTGCATGACCAGCTGCTGCAGCTGCCTGAAGGGCT | |
| | GCTGCAGCTGCGGCAGCTGCTGCAAGTTCGACGAGGACGACAGCGAGCCC | |
| | GTGCTGAAGGGCGTGAAGCTGCACTACACCGGAAGCGGAGCCACGAACTT | |
| | CTCTCTGTTAAAGCAAGCAGGAGATGTTGAAGAAAACCCCGGGCCTATGAA | |
| | GGCAATACTAGTAGTTCTGCTATATACATTTGCAACCGCAAATGCAGACACA | |
| | TTATGTATAGGTTATCATGCGAACAACTCAACAGACACTGTAGACACAGTAC | |
| | TAGAAAAGAATGTAACAGTAACACACTCTGTTAACCTTCTAGAAGACAAGCA | |
| | TAACGGGAAACTATGCAAACTAAGAGGGGTAGCCCCATTGCATTTGGGTAA | |
| | ATGTAACATTGCTGGCTGGATCCTGGGAAATCCAGAGTGTGAATCACTCTC | |
| | CACAGCAAGCTCATGGTCCTACATTGTGGAAACATCTAGTTCAGACAATGGA | |
| | ACGTGTTACCCAGGAGATTTCATCGATTATGAGGAGCTAAGAGAGCAATTGA | |
| | GCTCAGTGTCATCATTTGAAAGGTTTGAGATATTCCCCAAGACAAGTTCATG | |
| | GCCCAATCATGACTCGAACAAAGGTGTAACGACAGCATGTCCTCATGCTGG | |
| | AGCAAAAAGCTTCTACAAAAATTTAATATGGCTAGTTAAAAAAGGAAATTCAT | |
| | ACCCAAAGCTCAGCAAATCCTACATTAATGATAAAGGGAAAGAAGTCCTCGT | |
| | GCTATGGGGCATTCACCATCCACCTACTAGTGCTGACCAACAAAGTCTCTAT | |
| | CAGAATGCAGATGCATATGTTTTTGTGGGGACATCAAGATACAGCAAGAAGT | |
| | TCAAGCCGGAAATAGCAATAAGGCCCAAAGTGAGGGATCAAGAAGGGAGAA | |
| | TGAACTATTACTGGACACTAGTAGAGCCGGGAGACAAAATAACATTCGAAG | |
| | CAACTGGAAATCTAGTGGTACCGAGATATGCATTCGCAATGGAAAGAAATG | |
| | CTGGATCTGGTATTATCATTTCAGATACACCAGTCCACGATTGCAATACAAC | |
| | TTGTCAGACACCCAAGGGTGCTATAAACACCAGCCTCCCATTTCAGAACATA | |
| | CATCCGATCACAATTGGAAAATGTCCAAAATATGTAAAAAGCACAAAATTGA | |
| | GACTGGCCACAGGATTGAGGAATGTCCCGTCCATTCAATCTAGAGGCCTAT | |
| | TTGGGGCCATTGCCGGTTTCATTGAAGGGGGGTGGACAGGGATGGTAGAT | |
| | GGATGGTACGGTTATCACCATCAAAATGAGCAGGGGTCAGGATATGCAGCC | |
| | GACCTGAAGAGCACACAGAATGCCATTGACGAGATTACTAACAAAGTAAACT | |
| | CTGTTATTGAAAAAATGAATACACAGTTCACAGCAGTAGGTAAAGAGTTCAA | |
| | CCACCTGGAAAAAAGAATAGAGAATTTAAATAAAAAAGTTGATGATGGTTTC | |
| | CTGGACATTTGGACTTACAATGCCGAACTGTTGGTTCTATTGGAAAATGAAA | |
| | GAACTTTGGACTACCACGATTCAAAGGTGAAGAACTTATATGAAAAGGTAAG | |
| | AAGCCAGTTAAAAAACAATGCCAAGGAAATTGGAAACGGCTGCTTTGAATTT | |
| | TACCACAAATGTGATAACACGTGCATGGAAAGTGTCAAAAATGGGACTTATG | |
| | ACTACCCAAAATACTCAGAGGAAGCAAAATTAAACAGAGAAGAAATAGATGG | |
| | GGTAAAGCTGGAATCAACAAGGATTTACCAGATTTTGGCGATCTATTCAACC | |
| | GTCGCCAGTTCATTGGTACTGGTAGTCTCCCTGGGGGCAATCAGTTTCTGG | |
| | ATGTGCTCTAATGGGTCTCTACAGTGTAGAATATGTATTTAAGGAAGCGGAG | |
| | CCACGAACTTCTCTCTGTTAAAGCAAGCAGGAGATGTTGAAGAAAACCCCG | |
| | GGCCTAGCAAAAGCAGGGGATAATTCTATTAACCATGAAGACTATCATTGCT | |
| | TTGAGCTACATTCTATGTCTGGTTTTCGCTCAAAAACTTCCTGGAAATGACAA | |
| | TAGCACTGCAACGCTGTGCCTTGGGCACCATGCAGTACCAAACGGAACGAT | |
| | AGTGAAAACAATCACGAATGACCAAATTGAAGTTACTAATGCTACTGAGCTG | |
| | GTTCAGAATTCCTCAATAGGTGAAATATGCGACAGTCCTCATCAGATCCTTG | |
| | ATGGAGAAAACTGCACACTAATAGATGCTCTATTGGGAGACCCTCAGTGTG | |
| | ATGGCTTTCAAAATAAGAAATGGGACCTTTTTGTTGAACGAAGCAAAGCCTA | |
| | CAGCAACTGTTACCCTTATGATGTGCCGGATTATGCCTCCCTTAGGTCACTA | |
| | GTTGCCTCATCCGGCACACTGGAGTTTAACAATGAAAGCTTCAATTGGGCT | |
| | GGAGTCACTCAAAACGGAACAAGTTCTGCTTGCATAAGGGGATCTAATAGTA | |
| | GTTTCTTTAGTAGATTAAATTGGTTGACCCACTTAAACTTCAAGTACCCAGCA | |
| | TTGAACGTGACTATGCCAAACAATGAACAATTTGACAAATTGTACATTTGGG | |
| | GGGTTCACCACCCGGGTACGGACAAGGACCAAATCTTCCTGTATGCTCAAT | |
| | CATCAGGAAGAATCACAGTATCTACCAAAAGAAGCCAACAAGCTGTAATCCC | |
| | GAATATCGGATCTAGACCCAGAATAAGGAATATCCCTAGCAGAATAAGCATC | |
| | TATTGGACAATAGTAAAACCGGGAGACATACTTTTGATTAACAGCACAGGGA | |
| | ATCTAATTGCTCCTAGGGGTTACTTCAAAATACGAAGTGGGAAAAGCTCAAT | |
| | AATGAGATCAGATGCACCCATTGGCAAATGCAAGTCTGAATGCATCACTCCA | |
| | AATGGAAGCATTCCCAATGACAAACCATTCCAAAATGTAAACAGGATCACAT | |
| | ACGGGGCCTGTCCCAGATATGTTAAGCAAAGCACTCTGAAATTGGCAACAG | |
| | GAATGCGAAATGTACCAGAGAAACAAACTAGAGGCATATTTGGCGCAATAG | |
| | CGGGTTTCATAGAAAATGGTTGGGAGGGAATGGTGGATGGTTGGTACGGTT | |
| | TCAGGCATCAAAATTCTGAGGGAAGAGGACAAGCAGCAGATCTCAAAAGCA | |
| | CTCAAGCAGCAATCGATCAAATCAATGGGAAGCTGAATCGATTGATCGGGA | |
| | AAACCAACGAGAAATTCCATCAGATTGAGAAAGAATTCTCAGAAGTAGAAGG | |
| | GAGAATTCAGGACCTTGAGAAATATGTTGAGGACACAAAAATAGATCTCTGG | |
| | TCATACAACGCAGAGCTTCTTGTTGCCCTGGAAAACCAACATACAATTGATC | |
| | TAACTGACTCAGAAATGAACAAACTGTTTGAAAAAACAAAGAAGCAACTGAG | |
| | GGAAAATGCTGAGGATATGGGCAATGGTTGTTTCAAAATATACCACAAATGT | |
| | GACAATGCCTGCATAGGATCAATCAGAAATGGAACTTATGACCACGATGTAT | |
| | ACAGGGATGAAGCATTAAACAACCGGTTCCAGATCAAGGGAGTTGAGCTGA | |
| | AGTCAGGGTACAAAGATTGGATCCTATGGATTTCCTTTGCCATATCATGTTTT | |
| | TTGCTTTGTGTTGCTTTGTTGGGGTTCATCATGTGGGCCTGCCAAAAGGGC | |
| | AACATTAGGTGCAACATTTGCATTTGAGTGCATTAATTAAAAACACCCTTGTT | |
| | TCTACTGGAAGCGGAGCCACGAACTTCTCTCTGTTAAAGCAAGCAGGAGAT | |
| | GTTGAAGAAAACCCCGGGCCTATTTTCTAATATCCACAAAATGAAGGCAATA | |
| | ATTGTACTACTCATGGTAGTAACATCCAATGCAGATCGAATCTGCACTGGGA | |
| | TAACATCGTCAAACTCACCACATGTCGTCAAAACTGCTACTCAAGGGGAGGT | |
| | CAACGTGACCGGTGTAATACCACTGACAACAACACCCACCAAATCTCATTTT | |
| | GCAAATCTCAAAGGAACAGAAACCAGGGGGAAACTATGCCCAAAATGCCTC | |
| | AACTGCACAGATCTGGATGTAGCCTTGGGCAGACCAAAATGCACAGGGAAA | |
| | ATACCCTCTGCAAGGGTTTCAATACTCCATGAAGTCAGACCTGTTACATCTG | |
| | GGTGCTTTCCTATAATGCACGATAGAACAAAAATTAGACAGCTGCCTAACCT | |
| | TCTCCGAGGATACGAACATGTCAGGTTATCAACTCACAACGTTATCAATGCA | |
| | AAAGATGCACCAGGAAGACCCTACGAAATTGGAACCTCAGGGTCTTGCCCT | |
| | AACATTACCAATGGAAACGGATTCTTCGCAACAATGGCTTGGGCCGTCCCA | |
| | AAAAACAAAACAGCAACAAATCCATTAACAATAGAAGTACCATACATTTGTAC | |
| | AGAAGGAGAAGACCAAATTACCGTTTGGGGGTTCCACTCTGACAACGAGAC | |
| | CCAAATGGCAAAGCTCTATGGGGACTCAAAGCCCCAGAAGTTCACCTCATC | |
| | TGCCAACGGAGTGACCACACATTACGTTTCACAGATTGGTGGCTTCCCAAAT | |
| | CAAACAGAAGACGGAGGACTACCACAAAGTGGCAGAATTGTTGTTGATTAC | |
| | ATGGTGCAGAAATCTGGAAAAACAGGAACAATTACCTATCAAAGAGGTATTT | |
| | TATTGCCTCAAAAAGTGTGGTGCGCAAGTGGCAGGAGCAAGGTAATAAAAG | |
| | GATCCTTGCCCTTAATTGGAGAAGCAGATTGCCTCCATGAAAAATACGGTG | |
| | GATTAAACAAAAGCAAGCCTTACTACACAGGGGAACATGCAAAGGCCATAG | |
| | GAAATTGCCCAATATGGGTGAAAACACCCTTGAAGCTGGCCAATGGAACCA | |
| | AATATAGACCCCCTGCAAAACTATTAAAGGAAAGAGGTTTCTTCGGAGCCAT | |
| | TGCTGGTTTCTTAGAGGGAGGATGGGAAGGAATGATTGCAGGTTGGCACG | |
| | GATACACATCCCATGGGGCACATGGAGTAGCGGTGGCAGCTGACCTTAAGA | |
| | GCACTCAAGAGGCCATAAACAAGATAACAAAAAATCTCAACTCTTTGAGTGA | |
| | GCTGGAAGTAAAGAATCTTCAAAGACTAAGCGGTGCCATGGATGAACTCCA | |
| | CAACGAAATACTAGAACTAGATGAGAAAGTGGATGATCTCAGAGCTGACAC | |
| | AATAAGCTCACAAATAGAACTCGCAGTCCTGCTTTCCAATGAAGGAATAATA | |
| | AACAGTGAAGATGAACATCTCTTGGCGCTTGAAAGAAAGCTGAAGAAAATG | |
| | CTGGGCCCCTCTGCTGTAGAGATAGGGAATGGATGCTTTGAAACCAAACAC | |
| | AAGTGCAACCAGACCTGCCTCGACAGAATAGCTGCTGGTACCTTTGATGCA | |
| | GGAGAATTTTCTCTCCCCACCTTTGATTCACTGAATATTACTGCTGCATCTTT | |
| | AAATGACGACGGATTGGACAATCATACTATACTGCTTTACTACTCAACTGCT | |
| | GCCTCCAGTTTGGCTGTAACACTGATGATAGCTATCTTTGTTGTTTATATGGT | |
| | CTCCAGAGACAATGTTTCTTGCTCCATTTGTCTATAAGGAAAGTTAAGCCCT | |
| | GTATTTTCCTTTATTGTAGTGCTTGTTTGCTTGTTGTCATTACAAAGAAACGT | |
| | TATTGAAAAATGGAAGCGGAGCCACGAACTTCTCTCTGTTAAAGCAAGCAG | |
| | GAGATGTTGAAGAAAACCCCGGGCCTTTCGGAGCTATTGCTGGTTTCTTGG | |
| | AAGGAGGATGGGAAGGAATGATTGCAGGTTGGCACGGATACACATCTCATG | |
| | GAGCACATGGAGTAGCAGTGGCAGCAGACCTTAAGAGTACCCATGATGACT | |
| | CGAGCTGGTACTGCATGCACGCAATGCTAGCTGCCCCTTTCCCGTCCTGGG | |
| | TACCCCGAGTCTCCCCCGACCTCGGGTCCCAGGTATGCTCCCACCTCCACC | |
| | TGCCCCACTCACCACCTCTGCTAGTTCCAGACACCTCCCAAGCACGCAGCA | |
| | ATGCAGCTCAAAACGCTTAGCCTAGCCACACCCCCACGGGAAACAGCAGTG | |
| | ATTAACCTTTAGCAATAAACGAAAGTTTAACTAAGCTATACTAACCCCAGGGT | |
| | TGGTCAATTTCGTGCCAGCCACACCCTGGAGCTAGCAAAAAAAA | |
| Pan-CoV- | TAGTTATTAATAGTAATCAATTACGGGGTCATTAGTTCATAGCCCATATATGG | 31 |
| Flu | AGTTCCGCGTTACATAACTTACGGTAAATGGCCCGCCTGGCTGACCGCCCA | |
| Vaccine | ACGACCCCCGCCCATTGACGTCAATAATGACGTATGTTCCCATAGTAACGC | |
| #11 CoV- | CAATAGGGACTTTCCATTGACGTCAATGGGTGGAGTATTTACGGTAAACTGC | |
| Wuhan | CCACTTGGCAGTACATCAAGTGTATCATATGCCAAGTACGCCCCCTATTGAC | |
| Spike | GTCAATGACGGTAAATGGCCCGCCTGGCATTATGCCCAGTACATGACCTTA | |
| (HexaPro)- | TGGGACTTTCCTACTTGGCAGTACATCTACGTATTAGTCATCGCTATTACCA | |
| Influenza | TGGTGATGCGGTTTTGGCAGTACATCAATGGGCGTGGATAGCGGTTTGACT | |
| (HA) | CACGGGGATTTCCAAGTCTCCACCCCATTGACGTCAATGGGAGTTTGTTTTG | |
| | GCACCAAAATCAACGGGACTTTCCAAAATGTCGTAACAACTCCGCCCCATTG | |
| | ACGCAAATGGGGGTAGGCGTGTACGGTGGGAGGTCTATATAAGCAGAGC | |
| | TGGTTTAGTGAACCGTCAGATCGAGAATAAACTAGTATTCTTCTGGTCCCCA | |
| | CAGACTCAGAGAGAACCCGCCACCATGTTCGTGTTCCTGGTGCTGCTGCCC | |
| | CTGGTGAGCAGCCAGTGCGTGAACCTGACCACCAGGACCCAGCTGCCCCC | |
| | CGCCTACACCAACAGCTTCACCAGGGGCGTGTACTACCCCGACAAGGTGTT | |
| | CAGGAGCAGCGTGCTGCACAGCACCCAGGACCTGTTCCTGCCCTTCTTCAG | |
| | CAACGTGACCTGGTTCCACGCCATCCACGTGAGCGGCACCAACGGCACCA | |
| | AGAGGTTCGACAACCCCGTGCTGCCCTTCAACGACGGCGTGTACTTCGCCA | |
| | GCACCGAGAAGAGCAACATCATCAGGGGCTGGATCTTCGGCACCACCCTG | |
| | GACAGCAAGACCCAGAGCCTGCTGATCGTGAACAACGCCACCAACGTGGT | |
| | GATCAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTACAG | |
| | CAGCGCCAACAACTGCACCTTCGAGTACGTGAGCCAGCCCTTCCTGATGGA | |
| | CCTGGAGGGCAAGCAGGGCAACTTCAAGAACCTGAGGGAGTTCGTGTTCA | |
| | AGAACATCGACGGCTACTTCAAGATCTACAGCAAGCACACCCCCATCAACC | |
| | TGGTGAGGGACCTGCCCCAGGGCTTCAGCGCCCTGGAGCCCCTGGTGGAC | |
| | CTGCCCATCGGCATCAACATCACCAGGTTCCAGACCCTGCTGGCCCTGCAC | |
| | AGGAGCTACCTGACCCCCGGCGACAGCAGCAGCGGCTGGACCGCCGGCG | |
| | CCGCCGCCTACTACGTGGGCTACCTGCAGCCCAGGACCTTCCTGCTGAAG | |
| | TACAACGAGAACGGCACCATCACCGACGCCGTGGACTGCGCCCTGGACCC | |
| | CCTGAGCGAGACCAAGTGCACCCTGAAGAGCTTCACCGTGGAGAAGGGCA | |
| | TCTACCAGACCAGCAACTTCAGGGTGCAGCCCACCGAGAGCATCGTGAGG | |
| | TTCCCCAACATCACCAACCTGTGCCCCTTCGGCGAGGTGTTCAACGCCACC | |
| | AGGTTCGCCAGCGTGTACGCCTGGAACAGGAAGAGGATCAGCAACTGCGT | |
| | GGCCGACTACAGCGTGCTGTACAACAGCGCCAGCTTCAGCACCTTCAAGTG | |
| | CTACGGCGTGAGCCCCACCAAGCTGAACGACCTGTGCTTCACCAACGTGTA | |
| | CGCCGACAGCTTCGTGATCAGGGGCGACGAGGTGAGGCAGATCGCCCCC | |
| | GGCCAGACCGGCAAGATCGCCGACTACAACTACAAGCTGCCCGACGACTT | |
| | CACCGGCTGCGTGATCGCCTGGAACAGCAACAACCTGGACAGCAAGGTGG | |
| | GCGGCAACTACAACTACCTGTACAGGCTGTTCAGGAAGAGCAACCTGAAGC | |
| | CCTTCGAGAGGGACATCAGCACCGAGATCTACCAGGCCGGCAGCACCCCC | |
| | TGCAACGGCGTGGAGGGCTTCAACTGCTACTTCCCCCTGCAGAGCTACGG | |
| | CTTCCAGCCCACCAACGGCGTGGGCTACCAGCCCTACAGGGTGGTGGTGC | |
| | TGAGCTTCGAGCTGCTGCACGCCCCCGCCACCGTGTGCGGCCCCAAGAAG | |
| | AGCACCAACCTGGTGAAGAACAAGTGCGTGAACTTCAACTTCAACGGCCTG | |
| | ACCGGCACCGGCGTGCTGACCGAGAGCAACAAGAAGTTCCTGCCCTTCCA | |
| | GCAGTTCGGCAGGGACATCGCCGACACCACCGACGCCGTGAGGGACCCC | |
| | CAGACCCTGGAGATCCTGGACATCACCCCCTGCAGCTTCGGCGGCGTGAG | |
| | CGTGATCACCCCCGGCACCAACACCAGCAACCAGGTGGCCGTGCTGTACC | |
| | AGGACGTGAACTGCACCGAGGTGCCCGTGGCCATCCACGCCGACCAGCTG | |
| | ACCCCCACCTGGAGGGTGTACAGCACCGGCAGCAACGTGTTCCAGACCAG | |
| | GGCCGGCTGCCTGATCGGCGCCGAGCACGTGAACAACAGCTACGAGTGCG | |
| | ACATCCCCATCGGCGCCGGCATCTGCGCCAGCTACCAGACCCAGACCAAC | |
| | AGCCCCGGCAGCGCCAGCAGCGTGGCCAGCCAGAGCATCATCGCCTACAC | |
| | CATGAGCCTGGGCGCCGAGAACAGCGTGGCCTACAGCAACAACAGCATCG | |
| | CCATCCCCACCAACTTCACCATCAGCGTGACCACCGAGATCCTGCCCGTGA | |
| | GCATGACCAAGACCAGCGTGGACTGCACCATGTACATCTGCGGCGACAGC | |
| | ACCGAGTGCAGCAACCTGCTGCTGCAGTACGGCAGCTTCTGCACCCAGCT | |
| | GAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACT | |
| | TCGGCGGCTTCAACTTCAGCCAGATCCTGCCCGACCCCAGCAAGCCCAGC | |
| | AAGAGGAGCCCCATCGAGGACCTGCTGTTCAACAAGGTGACCCTGGCCGA | |
| | CGCCGGCTTCATCAAGCAGTACGGCGACTGCCTGGGCGACATCGCCGCCA | |
| | GGGACCTGATCTGCGCCCAGAAGTTCAACGGCCTGACCGTGCTGCCCCCC | |
| | CTGCTGACCGACGAGATGATCGCCCAGTACACCAGCGCCCTGCTGGCCGG | |
| | CACCATCACCAGCGGCTGGACCTTCGGCGCCGGCCCCGCCCTGCAGATCC | |
| | CCTTCCCCATGCAGATGGCCTACAGGTTCAACGGCATCGGCGTGACCCAGA | |
| | ACGTGCTGTACGAGAACCAGAAGCTGATCGCCAACCAGTTCAACAGCGCCA | |
| | TCGGCAAGATCCAGGACAGCCTGAGCAGCACCCCCAGCGCCCTGGGCAAG | |
| | CTGCAGGACGTGGTGAACCAGAACGCCCAGGCCCTGAACACCCTGGTGAA | |
| | GCAGCTGAGCAGCAACTTCGGCGCCATCAGCAGCGTGCTGAACGACATCC | |
| | TGAGCAGGCTGGACCCCCCCGAGGCCGAGGTGCAGATCGACAGGCTGATC | |
| | ACCGGCAGGCTGCAGAGCCTGCAGACCTACGTGACCCAGCAGCTGATCAG | |
| | GGCCGCCGAGATCAGGGCCAGCGCCAACCTGGCCGCCACCAAGATGAGC | |
| | GAGTGCGTGCTGGGCCAGAGCAAGAGGGTGGACTTCTGCGGCAAGGGCTA | |
| | CCACCTGATGAGCTTCCCCCAGAGCGCCCCCCACGGCGTGGTGTTCCTGC | |
| | ACGTGACCTACGTGCCCGCCCAGGAGAAGAACTTCACCACCGCCCCCGCC | |
| | ATCTGCCACGACGGCAAGGCCCACTTCCCCAGGGAGGGCGTGTTCGTGAG | |
| | CAACGGCACCCACTGGTTCGTGACCCAGAGGAACTTCTACGAGCCCCAGAT | |
| | CATCACCACCGACAACACCTTCGTGAGCGGCAACTGCGACGTGGTGATCG | |
| | GCATCGTGAACAACACCGTGTACGACCCCCTGCAGCCCGAGCTGGACAGC | |
| | TTCAAGGAGGAGCTGGACAAGTACTTCAAGAACCACACCAGCCCCGACGTG | |
| | GACCTGGGCGACATCAGCGGCATCAACGCCAGCGTGGTGAACATCCAGAA | |
| | GGAGATCGACAGGCTGAACGAGGTGGCCAAGAACCTGAACGAGAGCCTGA | |
| | TCGACCTGCAGGAGCTGGGCAAGTACGAGCAGTACATCAAGTGGCCCTGG | |
| | TACATCTGGCTGGGCTTCATCGCCGGCCTGATCGCCATCGTGATGGTGACC | |
| | ATCATGCTGTGCTGCATGACCAGCTGCTGCAGCTGCCTGAAGGGCTGCTGC | |
| | AGCTGCGGCAGCTGCTGCAAGTTCGACGAGGACGACAGCGAGCCCGTGCT | |
| | GAAGGGCGTGAAGCTGCACTACACCGGAAGCGGAGCCACGAACTTCTCTC | |
| | TGTTAAAGCAAGCAGGAGATGTTGAAGAAAACCCCGGGCCTATGAAGGCAA | |
| | TACTAGTAGTTCTGCTATATACATTTGCAACCGCAAATGCAGACACATTATGT | |
| | ATAGGTTATCATGCGAACAACTCAACAGACACTGTAGACACAGTACTAGAAA | |
| | AGAATGTAACAGTAACACACTCTGTTAACCTTCTAGAAGACAAGCATAACGG | |
| | GAAACTATGCAAACTAAGAGGGGTAGCCCCATTGCATTTGGGTAAATGTAAC | |
| | ATTGCTGGCTGGATCCTGGGAAATCCAGAGTGTGAATCACTCTCCACAGCA | |
| | AGCTCATGGTCCTACATTGTGGAAACATCTAGTTCAGACAATGGAACGTGTT | |
| | ACCCAGGAGATTTCATCGATTATGAGGAGCTAAGAGAGCAATTGAGCTCAG | |
| | TGTCATCATTTGAAAGGTTTGAGATATTCCCCAAGACAAGTTCATGGCCCAA | |
| | TCATGACTCGAACAAAGGTGTAACGACAGCATGTCCTCATGCTGGAGCAAA | |
| | AAGCTTCTACAAAAATTTAATATGGCTAGTTAAAAAAGGAAATTCATACCCAA | |
| | AGCTCAGCAAATCCTACATTAATGATAAAGGGAAAGAAGTCCTCGTGCTATG | |
| | GGGCATTCACCATCCACCTACTAGTGCTGACCAACAAAGTCTCTATCAGAAT | |
| | GCAGATGCATATGTTTTTGTGGGGACATCAAGATACAGCAAGAAGTTCAAGC | |
| | CGGAAATAGCAATAAGGCCCAAAGTGAGGGATCAAGAAGGGAGAATGAACT | |
| | ATTACTGGACACTAGTAGAGCCGGGAGACAAAATAACATTCGAAGCAACTG | |
| | GAAATCTAGTGGTACCGAGATATGCATTCGCAATGGAAAGAAATGCTGGAT | |
| | CTGGTATTATCATTTCAGATACACCAGTCCACGATTGCAATACAACTTGTCA | |
| | GACACCCAAGGGTGCTATAAACACCAGCCTCCCATTTCAGAACATACATCC | |
| | GATCACAATTGGAAAATGTCCAAAATATGTAAAAAGCACAAAATTGAGACTG | |
| | GCCACAGGATTGAGGAATGTCCCGTCCATTCAATCTAGAGGCCTATTTGGG | |
| | GCCATTGCCGGTTTCATTGAAGGGGGGTGGACAGGGATGGTAGATGGATG | |
| | GTACGGTTATCACCATCAAAATGAGCAGGGGTCAGGATATGCAGCCGACCT | |
| | GAAGAGCACACAGAATGCCATTGACGAGATTACTAACAAAGTAAACTCTGTT | |
| | ATTGAAAAAATGAATACACAGTTCACAGCAGTAGGTAAAGAGTTCAACCACC | |
| | TGGAAAAAAGAATAGAGAATTTAAATAAAAAAGTTGATGATGGTTTCCTGGA | |
| | CATTTGGACTTACAATGCCGAACTGTTGGTTCTATTGGAAAATGAAAGAACT | |
| | TTGGACTACCACGATTCAAAGGTGAAGAACTTATATGAAAAGGTAAGAAGCC | |
| | AGTTAAAAAACAATGCCAAGGAAATTGGAAACGGCTGCTTTGAATTTTACCA | |
| | CAAATGTGATAACACGTGCATGGAAAGTGTCAAAAATGGGACTTATGACTAC | |
| | CCAAAATACTCAGAGGAAGCAAAATTAAACAGAGAAGAAATAGATGGGGTAA | |
| | AGCTGGAATCAACAAGGATTTACCAGATTTTGGCGATCTATTCAACCGTCGC | |
| | CAGTTCATTGGTACTGGTAGTCTCCCTGGGGGCAATCAGTTTCTGGATGTG | |
| | CTCTAATGGGTCTCTACAGTGTAGAATATGTATTTAAGGAAGCGGAGCCACG | |
| | AACTTCTCTCTGTTAAAGCAAGCAGGAGATGTTGAAGAAAACCCCGGGCCT | |
| | AGCAAAAGCAGGGGATAATTCTATTAACCATGAAGACTATCATTGCTTTGAG | |
| | CTACATTCTATGTCTGGTTTTCGCTCAAAAACTTCCTGGAAATGACAATAGCA | |
| | CTGCAACGCTGTGCCTTGGGCACCATGCAGTACCAAACGGAACGATAGTGA | |
| | AAACAATCACGAATGACCAAATTGAAGTTACTAATGCTACTGAGCTGGTTCA | |
| | GAATTCCTCAATAGGTGAAATATGCGACAGTCCTCATCAGATCCTTGATGGA | |
| | GAAAACTGCACACTAATAGATGCTCTATTGGGAGACCCTCAGTGTGATGGC | |
| | TTTCAAAATAAGAAATGGGACCTTTTTGTTGAACGAAGCAAAGCCTACAGCA | |
| | ACTGTTACCCTTATGATGTGCCGGATTATGCCTCCCTTAGGTCACTAGTTGC | |
| | CTCATCCGGCACACTGGAGTTTAACAATGAAAGCTTCAATTGGGCTGGAGT | |
| | CACTCAAAACGGAACAAGTTCTGCTTGCATAAGGGGATCTAATAGTAGTTTC | |
| | TTTAGTAGATTAAATTGGTTGACCCACTTAAACTTCAAGTACCCAGCATTGAA | |
| | CGTGACTATGCCAAACAATGAACAATTTGACAAATTGTACATTTGGGGGGTT | |
| | CACCACCCGGGTACGGACAAGGACCAAATCTTCCTGTATGCTCAATCATCA | |
| | GGAAGAATCACAGTATCTACCAAAAGAAGCCAACAAGCTGTAATCCCGAATA | |
| | TCGGATCTAGACCCAGAATAAGGAATATCCCTAGCAGAATAAGCATCTATTG | |
| | GACAATAGTAAAACCGGGAGACATACTTTTGATTAACAGCACAGGGAATCTA | |
| | ATTGCTCCTAGGGGTTACTTCAAAATACGAAGTGGGAAAAGCTCAATAATGA | |
| | GATCAGATGCACCCATTGGCAAATGCAAGTCTGAATGCATCACTCCAAATG | |
| | GAAGCATTCCCAATGACAAACCATTCCAAAATGTAAACAGGATCACATACGG | |
| | GGCCTGTCCCAGATATGTTAAGCAAAGCACTCTGAAATTGGCAACAGGAAT | |
| | GCGAAATGTACCAGAGAAACAAACTAGAGGCATATTTGGCGCAATAGCGGG | |
| | TTTCATAGAAAATGGTTGGGAGGGAATGGTGGATGGTTGGTACGGTTTCAG | |
| | GCATCAAAATTCTGAGGGAAGAGGACAAGCAGCAGATCTCAAAAGCACTCA | |
| | AGCAGCAATCGATCAAATCAATGGGAAGCTGAATCGATTGATCGGGAAAAC | |
| | CAACGAGAAATTCCATCAGATTGAGAAAGAATTCTCAGAAGTAGAAGGGAG | |
| | AATTCAGGACCTTGAGAAATATGTTGAGGACACAAAAATAGATCTCTGGTCA | |
| | TACAACGCAGAGCTTCTTGTTGCCCTGGAAAACCAACATACAATTGATCTAA | |
| | CTGACTCAGAAATGAACAAACTGTTTGAAAAAACAAAGAAGCAACTGAGGGA | |
| | AAATGCTGAGGATATGGGCAATGGTTGTTTCAAAATATACCACAAATGTGAC | |
| | AATGCCTGCATAGGATCAATCAGAAATGGAACTTATGACCACGATGTATACA | |
| | GGGATGAAGCATTAAACAACCGGTTCCAGATCAAGGGAGTTGAGCTGAAGT | |
| | CAGGGTACAAAGATTGGATCCTATGGATTTCCTTTGCCATATCATGTTTTTTG | |
| | CTTTGTGTTGCTTTGTTGGGGTTCATCATGTGGGCCTGCCAAAAGGGCAAC | |
| | ATTAGGTGCAACATTTGCATTTGAGTGCATTAATTAAAAACACCCTTGTTTCT | |
| | ACTGGAAGCGGAGCCACGAACTTCTCTCTGTTAAAGCAAGCAGGAGATGTT | |
| | GAAGAAAACCCCGGGCCTATTTTCTAATATCCACAAAATGAAGGCAATAATT | |
| | GTACTACTCATGGTAGTAACATCCAATGCAGATCGAATCTGCACTGGGATAA | |
| | CATCGTCAAACTCACCACATGTCGTCAAAACTGCTACTCAAGGGGAGGTCA | |
| | ACGTGACCGGTGTAATACCACTGACAACAACACCCACCAAATCTCATTTTGC | |
| | AAATCTCAAAGGAACAGAAACCAGGGGGAAACTATGCCCAAAATGCCTCAA | |
| | CTGCACAGATCTGGATGTAGCCTTGGGCAGACCAAAATGCACAGGGAAAAT | |
| | ACCCTCTGCAAGGGTTTCAATACTCCATGAAGTCAGACCTGTTACATCTGGG | |
| | TGCTTTCCTATAATGCACGATAGAACAAAAATTAGACAGCTGCCTAACCTTC | |
| | TCCGAGGATACGAACATGTCAGGTTATCAACTCACAACGTTATCAATGCAAA | |
| | AGATGCACCAGGAAGACCCTACGAAATTGGAACCTCAGGGTCTTGCCCTAA | |
| | CATTACCAATGGAAACGGATTCTTCGCAACAATGGCTTGGGCCGTCCCAAA | |
| | AAACAAAACAGCAACAAATCCATTAACAATAGAAGTACCATACATTTGTACAG | |
| | AAGGAGAAGACCAAATTACCGTTTGGGGGTTCCACTCTGACAACGAGACCC | |
| | AAATGGCAAAGCTCTATGGGGACTCAAAGCCCCAGAAGTTCACCTCATCTG | |
| | CCAACGGAGTGACCACACATTACGTTTCACAGATTGGTGGCTTCCCAAATCA | |
| | AACAGAAGACGGAGGACTACCACAAAGTGGCAGAATTGTTGTTGATTACAT | |
| | GGTGCAGAAATCTGGAAAAACAGGAACAATTACCTATCAAAGAGGTATTTTA | |
| | TTGCCTCAAAAAGTGTGGTGCGCAAGTGGCAGGAGCAAGGTAATAAAAGGA | |
| | TCCTTGCCCTTAATTGGAGAAGCAGATTGCCTCCATGAAAAATACGGTGGAT | |
| | TAAACAAAAGCAAGCCTTACTACACAGGGGAACATGCAAAGGCCATAGGAA | |
| | ATTGCCCAATATGGGTGAAAACACCCTTGAAGCTGGCCAATGGAACCAAAT | |
| | ATAGACCCCCTGCAAAACTATTAAAGGAAAGAGGTTTCTTCGGAGCCATTGC | |
| | TGGTTTCTTAGAGGGAGGATGGGAAGGAATGATTGCAGGTTGGCACGGATA | |
| | CACATCCCATGGGGCACATGGAGTAGCGGTGGCAGCTGACCTTAAGAGCA | |
| | CTCAAGAGGCCATAAACAAGATAACAAAAAATCTCAACTCTTTGAGTGAGCT | |
| | GGAAGTAAAGAATCTTCAAAGACTAAGCGGTGCCATGGATGAACTCCACAA | |
| | CGAAATACTAGAACTAGATGAGAAAGTGGATGATCTCAGAGCTGACACAATA | |
| | AGCTCACAAATAGAACTCGCAGTCCTGCTTTCCAATGAAGGAATAATAAACA | |
| | GTGAAGATGAACATCTCTTGGCGCTTGAAAGAAAGCTGAAGAAAATGCTGG | |
| | GCCCCTCTGCTGTAGAGATAGGGAATGGATGCTTTGAAACCAAACACAAGT | |
| | GCAACCAGACCTGCCTCGACAGAATAGCTGCTGGTACCTTTGATGCAGGAG | |
| | AATTTTCTCTCCCCACCTTTGATTCACTGAATATTACTGCTGCATCTTTAAAT | |
| | GACGACGGATTGGACAATCATACTATACTGCTTTACTACTCAACTGCTGCCT | |
| | CCAGTTTGGCTGTAACACTGATGATAGCTATCTTTGTTGTTTATATGGTCTCC | |
| | AGAGACAATGTTTCTTGCTCCATTTGTCTATAAGGAAAGTTAAGCCCTGTATT | |
| | TTCCTTTATTGTAGTGCTTGTTTGCTTGTTGTCATTACAAAGAAACGTTATTG | |
| | AAAAATGGAAGCGGAGCCACGAACTTCTCTCTGTTAAAGCAAGCAGGAGAT | |
| | GTTGAAGAAAACCCCGGGCCTTTCGGAGCTATTGCTGGTTTCTTGGAAGGA | |
| | GGATGGGAAGGAATGATTGCAGGTTGGCACGGATACACATCTCATGGAGCA | |
| | CATGGAGTAGCAGTGGCAGCAGACCTTAAGAGTACCCATGATGACTCGAGC | |
| | TGGTACTGCATGCACGCAATGCTAGCTGCCCCTTTCCCGTCCTGGGTACCC | |
| | CGAGTCTCCCCCGACCTCGGGTCCCAGGTATGCTCCCACCTCCACCTGCC | |
| | CCACTCACCACCTCTGCTAGTTCCAGACACCTCCCAAGCACGCAGCAATGC | |
| | AGCTCAAAACGCTTAGCCTAGCCACACCCCCACGGGAAACAGCAGTGATTA | |
| | ACCTTTAGCAATAAACGAAAGTTTAACTAAGCTATACTAACCCCAGGGTTGG | |
| | TCAATTTCGTGCCAGCCACACCCTGGAGCTAGCAAAAAAAA | |
| Pan-CoV- | TAGTTATTAATAGTAATCAATTACGGGGTCATTAGTTCATAGCCCATATATGG | 32 |
| Flu | AGTTCCGCGTTACATAACTTACGGTAAATGGCCCGCCTGGCTGACCGCCCA | |
| Vaccine | ACGACCCCCGCCCATTGACGTCAATAATGACGTATGTTCCCATAGTAACGC | |
| #12 CoV- | CAATAGGGACTTTCCATTGACGTCAATGGGTGGAGTATTTACGGTAAACTGC | |
| Wuhan | CCACTTGGCAGTACATCAAGTGTATCATATGCCAAGTACGCCCCCTATTGAC | |
| Spike | GTCAATGACGGTAAATGGCCCGCCTGGCATTATGCCCAGTACATGACCTTA | |
| (2 proline | TGGGACTTTCCTACTTGGCAGTACATCTACGTATTAGTCATCGCTATTACCA | |
| subst.)- | TGGTGATGCGGTTTTGGCAGTACATCAATGGGCGTGGATAGCGGTTTGACT | |
| Influenza | CACGGGGATTTCCAAGTCTCCACCCCATTGACGTCAATGGGAGTTTGTTTTG | |
| (HA) | GCACCAAAATCAACGGGACTTTCCAAAATGTCGTAACAACTCCGCCCCATTG | |
| | ACGCAAATGGGGGGTAGGCGTGTACGGTGGGAGGTCTATATAAGCAGAGC | |
| | TGGTTTAGTGAACCGTCAGATCGAGAATAAACTAGTATTCTTCTGGTCCCCA | |
| | CAGACTCAGAGAGAACCCGCCACCATGTTCGTGTTCCTGGTGCTGCTGCCC | |
| | CTGGTGAGCAGCCAGTGCGTGAACCTGACCACCAGGACCCAGCTGCCCCC | |
| | CGCCTACACCAACAGCTTCACCAGGGGCGTGTACTACCCCGACAAGGTGTT | |
| | CAGGAGCAGCGTGCTGCACAGCACCCAGGACCTGTTCCTGCCCTTCTTCAG | |
| | CAACGTGACCTGGTTCCACGCCATCCACGTGAGCGGCACCAACGGCACCA | |
| | AGAGGTTCGACAACCCCGTGCTGCCCTTCAACGACGGCGTGTACTTCGCCA | |
| | GCACCGAGAAGAGCAACATCATCAGGGGCTGGATCTTCGGCACCACCCTG | |
| | GACAGCAAGACCCAGAGCCTGCTGATCGTGAACAACGCCACCAACGTGGT | |
| | GATCAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTACAG | |
| | CAGCGCCAACAACTGCACCTTCGAGTACGTGAGCCAGCCCTTCCTGATGGA | |
| | CCTGGAGGGCAAGCAGGGCAACTTCAAGAACCTGAGGGAGTTCGTGTTCA | |
| | AGAACATCGACGGCTACTTCAAGATCTACAGCAAGCACACCCCCATCAACC | |
| | TGGTGAGGGACCTGCCCCAGGGCTTCAGCGCCCTGGAGCCCCTGGTGGAC | |
| | CTGCCCATCGGCATCAACATCACCAGGTTCCAGACCCTGCTGGCCCTGCAC | |
| | AGGAGCTACCTGACCCCCGGCGACAGCAGCAGCGGCTGGACCGCCGGCG | |
| | CCGCCGCCTACTACGTGGGCTACCTGCAGCCCAGGACCTTCCTGCTGAAG | |
| | TACAACGAGAACGGCACCATCACCGACGCCGTGGACTGCGCCCTGGACCC | |
| | CCTGAGCGAGACCAAGTGCACCCTGAAGAGCTTCACCGTGGAGAAGGGCA | |
| | TCTACCAGACCAGCAACTTCAGGGTGCAGCCCACCGAGAGCATCGTGAGG | |
| | TTCCCCAACATCACCAACCTGTGCCCCTTCGGCGAGGTGTTCAACGCCACC | |
| | AGGTTCGCCAGCGTGTACGCCTGGAACAGGAAGAGGATCAGCAACTGCGT | |
| | GGCCGACTACAGCGTGCTGTACAACAGCGCCAGCTTCAGCACCTTCAAGTG | |
| | CTACGGCGTGAGCCCCACCAAGCTGAACGACCTGTGCTTCACCAACGTGTA | |
| | CGCCGACAGCTTCGTGATCAGGGGCGACGAGGTGAGGCAGATCGCCCCC | |
| | GGCCAGACCGGCAAGATCGCCGACTACAACTACAAGCTGCCCGACGACTT | |
| | CACCGGCTGCGTGATCGCCTGGAACAGCAACAACCTGGACAGCAAGGTGG | |
| | GCGGCAACTACAACTACCTGTACAGGCTGTTCAGGAAGAGCAACCTGAAGC | |
| | CCTTCGAGAGGGACATCAGCACCGAGATCTACCAGGCCGGCAGCACCCCC | |
| | TGCAACGGCGTGGAGGGCTTCAACTGCTACTTCCCCCTGCAGAGCTACGG | |
| | CTTCCAGCCCACCAACGGCGTGGGCTACCAGCCCTACAGGGTGGTGGTGC | |
| | TGAGCTTCGAGCTGCTGCACGCCCCCGCCACCGTGTGCGGCCCCAAGAAG | |
| | AGCACCAACCTGGTGAAGAACAAGTGCGTGAACTTCAACTTCAACGGCCTG | |
| | ACCGGCACCGGCGTGCTGACCGAGAGCAACAAGAAGTTCCTGCCCTTCCA | |
| | GCAGTTCGGCAGGGACATCGCCGACACCACCGACGCCGTGAGGGACCCC | |
| | CAGACCCTGGAGATCCTGGACATCACCCCCTGCAGCTTCGGCGGCGTGAG | |
| | CGTGATCACCCCCGGCACCAACACCAGCAACCAGGTGGCCGTGCTGTACC | |
| | AGGACGTGAACTGCACCGAGGTGCCCGTGGCCATCCACGCCGACCAGCTG | |
| | ACCCCCACCTGGAGGGTGTACAGCACCGGCAGCAACGTGTTCCAGACCAG | |
| | GGCCGGCTGCCTGATCGGCGCCGAGCACGTGAACAACAGCTACGAGTGCG | |
| | ACATCCCCATCGGCGCCGGCATCTGCGCCAGCTACCAGACCCAGACCAAC | |
| | AGCCCCAGGAGGGCCAGGAGCGTGGCCAGCCAGAGCATCATCGCCTACAC | |
| | CATGAGCCTGGGCGCCGAGAACAGCGTGGCCTACAGCAACAACAGCATCG | |
| | CCATCCCCACCAACTTCACCATCAGCGTGACCACCGAGATCCTGCCCGTGA | |
| | GCATGACCAAGACCAGCGTGGACTGCACCATGTACATCTGCGGCGACAGC | |
| | ACCGAGTGCAGCAACCTGCTGCTGCAGTACGGCAGCTTCTGCACCCAGCT | |
| | GAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACT | |
| | TCGGCGGCTTCAACTTCAGCCAGATCCTGCCCGACCCCAGCAAGCCCAGC | |
| | AAGAGGAGCTTCATCGAGGACCTGCTGTTCAACAAGGTGACCCTGGCCGAC | |
| | GCCGGCTTCATCAAGCAGTACGGCGACTGCCTGGGCGACATCGCCGCCAG | |
| | GGACCTGATCTGCGCCCAGAAGTTCAACGGCCTGACCGTGCTGCCCCCCC | |
| | TGCTGACCGACGAGATGATCGCCCAGTACACCAGCGCCCTGCTGGCCGGC | |
| | ACCATCACCAGCGGCTGGACCTTCGGCGCCGGCGCCGCCCTGCAGATCCC | |
| | CTTCGCCATGCAGATGGCCTACAGGTTCAACGGCATCGGCGTGACCCAGAA | |
| | CGTGCTGTACGAGAACCAGAAGCTGATCGCCAACCAGTTCAACAGCGCCAT | |
| | CGGCAAGATCCAGGACAGCCTGAGCAGCACCGCCAGCGCCCTGGGCAAG | |
| | CTGCAGGACGTGGTGAACCAGAACGCCCAGGCCCTGAACACCCTGGTGAA | |
| | GCAGCTGAGCAGCAACTTCGGCGCCATCAGCAGCGTGCTGAACGACATCC | |
| | TGAGCAGGCTGGACCCCCCCGAGGCCGAGGTGCAGATCGACAGGCTGATC | |
| | ACCGGCAGGCTGCAGAGCCTGCAGACCTACGTGACCCAGCAGCTGATCAG | |
| | GGCCGCCGAGATCAGGGCCAGCGCCAACCTGGCCGCCACCAAGATGAGC | |
| | GAGTGCGTGCTGGGCCAGAGCAAGAGGGTGGACTTCTGCGGCAAGGGCTA | |
| | CCACCTGATGAGCTTCCCCCAGAGCGCCCCCCACGGCGTGGTGTTCCTGC | |
| | ACGTGACCTACGTGCCCGCCCAGGAGAAGAACTTCACCACCGCCCCCGCC | |
| | ATCTGCCACGACGGCAAGGCCCACTTCCCCAGGGAGGGCGTGTTCGTGAG | |
| | CAACGGCACCCACTGGTTCGTGACCCAGAGGAACTTCTACGAGCCCCAGAT | |
| | CATCACCACCGACAACACCTTCGTGAGCGGCAACTGCGACGTGGTGATCG | |
| | GCATCGTGAACAACACCGTGTACGACCCCCTGCAGCCCGAGCTGGACAGC | |
| | TTCAAGGAGGAGCTGGACAAGTACTTCAAGAACCACACCAGCCCCGACGTG | |
| | GACCTGGGCGACATCAGCGGCATCAACGCCAGCGTGGTGAACATCCAGAA | |
| | GGAGATCGACAGGCTGAACGAGGTGGCCAAGAACCTGAACGAGAGCCTGA | |
| | TCGACCTGCAGGAGCTGGGCAAGTACGAGCAGTACATCAAGTGGCCCTGG | |
| | TACATCTGGCTGGGCTTCATCGCCGGCCTGATCGCCATCGTGATGGTGACC | |
| | ATCATGCTGTGCTGCATGACCAGCTGCTGCAGCTGCCTGAAGGGCTGCTGC | |
| | AGCTGCGGCAGCTGCTGCAAGTTCGACGAGGACGACAGCGAGCCCGTGCT | |
| | GAAGGGCGTGAAGCTGCACTACACCGGAAGCGGAGCCACGAACTTCTCTC | |
| | TGTTAAAGCAAGCAGGAGATGTTGAAGAAAACCCCGGGCCTATGAAGGCAA | |
| | TACTAGTAGTTCTGCTATATACATTTGCAACCGCAAATGCAGACACATTATGT | |
| | ATAGGTTATCATGCGAACAACTCAACAGACACTGTAGACACAGTACTAGAAA | |
| | AGAATGTAACAGTAACACACTCTGTTAACCTTCTAGAAGACAAGCATAACGG | |
| | GAAACTATGCAAACTAAGAGGGGTAGCCCCATTGCATTTGGGTAAATGTAAC | |
| | ATTGCTGGCTGGATCCTGGGAAATCCAGAGTGTGAATCACTCTCCACAGCA | |
| | AGCTCATGGTCCTACATTGTGGAAACATCTAGTTCAGACAATGGAACGTGTT | |
| | ACCCAGGAGATTTCATCGATTATGAGGAGCTAAGAGAGCAATTGAGCTCAG | |
| | TGTCATCATTTGAAAGGTTTGAGATATTCCCCAAGACAAGTTCATGGCCCAA | |
| | TCATGACTCGAACAAAGGTGTAACGACAGCATGTCCTCATGCTGGAGCAAA | |
| | AAGCTTCTACAAAAATTTAATATGGCTAGTTAAAAAAGGAAATTCATACCCAA | |
| | AGCTCAGCAAATCCTACATTAATGATAAAGGGAAAGAAGTCCTCGTGCTATG | |
| | GGGCATTCACCATCCACCTACTAGTGCTGACCAACAAAGTCTCTATCAGAAT | |
| | GCAGATGCATATGTTTTTGTGGGGACATCAAGATACAGCAAGAAGTTCAAGC | |
| | CGGAAATAGCAATAAGGCCCAAAGTGAGGGATCAAGAAGGGAGAATGAACT | |
| | ATTACTGGACACTAGTAGAGCCGGGAGACAAAATAACATTCGAAGCAACTG | |
| | GAAATCTAGTGGTACCGAGATATGCATTCGCAATGGAAAGAAATGCTGGAT | |
| | CTGGTATTATCATTTCAGATACACCAGTCCACGATTGCAATACAACTTGTCA | |
| | GACACCCAAGGGTGCTATAAACACCAGCCTCCCATTTCAGAACATACATCC | |
| | GATCACAATTGGAAAATGTCCAAAATATGTAAAAAGCACAAAATTGAGACTG | |
| | GCCACAGGATTGAGGAATGTCCCGTCCATTCAATCTAGAGGCCTATTTGGG | |
| | GCCATTGCCGGTTTCATTGAAGGGGGGGGACAGGGATGGTAGATGGATG | |
| | GTACGGTTATCACCATCAAAATGAGCAGGGGTCAGGATATGCAGCCGACCT | |
| | GAAGAGCACACAGAATGCCATTGACGAGATTACTAACAAAGTAAACTCTGTT | |
| | ATTGAAAAAATGAATACACAGTTCACAGCAGTAGGTAAAGAGTTCAACCACC | |
| | TGGAAAAAAGAATAGAGAATTTAAATAAAAAAGTTGATGATGGTTTCCTGGA | |
| | CATTTGGACTTACAATGCCGAACTGTTGGTTCTATTGGAAAATGAAAGAACT | |
| | TTGGACTACCACGATTCAAAGGTGAAGAACTTATATGAAAAGGTAAGAAGCC | |
| | AGTTAAAAAACAATGCCAAGGAAATTGGAAACGGCTGCTTTGAATTTTACCA | |
| | CAAATGTGATAACACGTGCATGGAAAGTGTCAAAAATGGGACTTATGACTAC | |
| | CCAAAATACTCAGAGGAAGCAAAATTAAACAGAGAAGAAATAGATGGGGTAA | |
| | AGCTGGAATCAACAAGGATTTACCAGATTTTGGCGATCTATTCAACCGTCGC | |
| | CAGTTCATTGGTACTGGTAGTCTCCCTGGGGGCAATCAGTTTCTGGATGTG | |
| | CTCTAATGGGTCTCTACAGTGTAGAATATGTATTTAAGGAAGCGGAGCCACG | |
| | AACTTCTCTCTGTTAAAGCAAGCAGGAGATGTTGAAGAAAACCCCGGGCCT | |
| | AGCAAAAGCAGGGGATAATTCTATTAACCATGAAGACTATCATTGCTTTGAG | |
| | CTACATTCTATGTCTGGTTTTCGCTCAAAAACTTCCTGGAAATGACAATAGCA | |
| | CTGCAACGCTGTGCCTTGGGCACCATGCAGTACCAAACGGAACGATAGTGA | |
| | AAACAATCACGAATGACCAAATTGAAGTTACTAATGCTACTGAGCTGGTTCA | |
| | GAATTCCTCAATAGGTGAAATATGCGACAGTCCTCATCAGATCCTTGATGGA | |
| | GAAAACTGCACACTAATAGATGCTCTATTGGGAGACCCTCAGTGTGATGGC | |
| | TTTCAAAATAAGAAATGGGACCTTTTTGTTGAACGAAGCAAAGCCTACAGCA | |
| | ACTGTTACCCTTATGATGTGCCGGATTATGCCTCCCTTAGGTCACTAGTTGC | |
| | CTCATCCGGCACACTGGAGTTTAACAATGAAAGCTTCAATTGGGCTGGAGT | |
| | CACTCAAAACGGAACAAGTTCTGCTTGCATAAGGGGATCTAATAGTAGTTTC | |
| | TTTAGTAGATTAAATTGGTTGACCCACTTAAACTTCAAGTACCCAGCATTGAA | |
| | CGTGACTATGCCAAACAATGAACAATTTGACAAATTGTACATTTGGGGGGTT | |
| | CACCACCCGGGTACGGACAAGGACCAAATCTTCCTGTATGCTCAATCATCA | |
| | GGAAGAATCACAGTATCTACCAAAAGAAGCCAACAAGCTGTAATCCCGAATA | |
| | TCGGATCTAGACCCAGAATAAGGAATATCCCTAGCAGAATAAGCATCTATTG | |
| | GACAATAGTAAAACCGGGAGACATACTTTTGATTAACAGCACAGGGAATCTA | |
| | ATTGCTCCTAGGGGTTACTTCAAAATACGAAGTGGGAAAAGCTCAATAATGA | |
| | GATCAGATGCACCCATTGGCAAATGCAAGTCTGAATGCATCACTCCAAATG | |
| | GAAGCATTCCCAATGACAAACCATTCCAAAATGTAAACAGGATCACATACGG | |
| | GGCCTGTCCCAGATATGTTAAGCAAAGCACTCTGAAATTGGCAACAGGAAT | |
| | GCGAAATGTACCAGAGAAACAAACTAGAGGCATATTTGGCGCAATAGCGGG | |
| | TTTCATAGAAAATGGTTGGGAGGGAATGGTGGATGGTTGGTACGGTTTCAG | |
| | GCATCAAAATTCTGAGGGAAGAGGACAAGCAGCAGATCTCAAAAGCACTCA | |
| | AGCAGCAATCGATCAAATCAATGGGAAGCTGAATCGATTGATCGGGAAAAC | |
| | CAACGAGAAATTCCATCAGATTGAGAAAGAATTCTCAGAAGTAGAAGGGAG | |
| | AATTCAGGACCTTGAGAAATATGTTGAGGACACAAAAATAGATCTCTGGTCA | |
| | TACAACGCAGAGCTTCTTGTTGCCCTGGAAAACCAACATACAATTGATCTAA | |
| | CTGACTCAGAAATGAACAAACTGTTTGAAAAAACAAAGAAGCAACTGAGGGA | |
| | AAATGCTGAGGATATGGGCAATGGTTGTTTCAAAATATACCACAAATGTGAC | |
| | AATGCCTGCATAGGATCAATCAGAAATGGAACTTATGACCACGATGTATACA | |
| | GGGATGAAGCATTAAACAACCGGTTCCAGATCAAGGGAGTTGAGCTGAAGT | |
| | CAGGGTACAAAGATTGGATCCTATGGATTTCCTTTGCCATATCATGTTTTTTG | |
| | CTTTGTGTTGCTTTGTTGGGGTTCATCATGTGGGCCTGCCAAAAGGGCAAC | |
| | ATTAGGTGCAACATTTGCATTTGAGTGCATTAATTAAAAACACCCTTGTTTCT | |
| | ACTGGAAGCGGAGCCACGAACTTCTCTCTGTTAAAGCAAGCAGGAGATGTT | |
| | GAAGAAAACCCCGGGCCTATTTTCTAATATCCACAAAATGAAGGCAATAATT | |
| | GTACTACTCATGGTAGTAACATCCAATGCAGATCGAATCTGCACTGGGATAA | |
| | CATCGTCAAACTCACCACATGTCGTCAAAACTGCTACTCAAGGGGAGGTCA | |
| | ACGTGACCGGTGTAATACCACTGACAACAACACCCACCAAATCTCATTTTGC | |
| | AAATCTCAAAGGAACAGAAACCAGGGGGAAACTATGCCCAAAATGCCTCAA | |
| | CTGCACAGATCTGGATGTAGCCTTGGGCAGACCAAAATGCACAGGGAAAAT | |
| | ACCCTCTGCAAGGGTTTCAATACTCCATGAAGTCAGACCTGTTACATCTGGG | |
| | TGCTTTCCTATAATGCACGATAGAACAAAAATTAGACAGCTGCCTAACCTTC | |
| | TCCGAGGATACGAACATGTCAGGTTATCAACTCACAACGTTATCAATGCAAA | |
| | AGATGCACCAGGAAGACCCTACGAAATTGGAACCTCAGGGTCTTGCCCTAA | |
| | CATTACCAATGGAAACGGATTCTTCGCAACAATGGCTTGGGCCGTCCCAAA | |
| | AAACAAAACAGCAACAAATCCATTAACAATAGAAGTACCATACATTTGTACAG | |
| | AAGGAGAAGACCAAATTACCGTTTGGGGGTTCCACTCTGACAACGAGACCC | |
| | AAATGGCAAAGCTCTATGGGGACTCAAAGCCCCAGAAGTTCACCTCATCTG | |
| | CCAACGGAGTGACCACACATTACGTTTCACAGATTGGTGGCTTCCCAAATCA | |
| | AACAGAAGACGGAGGACTACCACAAAGTGGCAGAATTGTTGTTGATTACAT | |
| | GGTGCAGAAATCTGGAAAAACAGGAACAATTACCTATCAAAGAGGTATTTTA | |
| | TTGCCTCAAAAAGTGTGGTGCGCAAGTGGCAGGAGCAAGGTAATAAAAGGA | |
| | TCCTTGCCCTTAATTGGAGAAGCAGATTGCCTCCATGAAAAATACGGTGGAT | |
| | TAAACAAAAGCAAGCCTTACTACACAGGGGAACATGCAAAGGCCATAGGAA | |
| | ATTGCCCAATATGGGTGAAAACACCCTTGAAGCTGGCCAATGGAACCAAAT | |
| | ATAGACCCCCTGCAAAACTATTAAAGGAAAGAGGTTTCTTCGGAGCCATTGC | |
| | TGGTTTCTTAGAGGGAGGATGGGAAGGAATGATTGCAGGTTGGCACGGATA | |
| | CACATCCCATGGGGCACATGGAGTAGCGGTGGCAGCTGACCTTAAGAGCA | |
| | CTCAAGAGGCCATAAACAAGATAACAAAAAATCTCAACTCTTTGAGTGAGCT | |
| | GGAAGTAAAGAATCTTCAAAGACTAAGCGGTGCCATGGATGAACTCCACAA | |
| | CGAAATACTAGAACTAGATGAGAAAGTGGATGATCTCAGAGCTGACACAATA | |
| | AGCTCACAAATAGAACTCGCAGTCCTGCTTTCCAATGAAGGAATAATAAACA | |
| | GTGAAGATGAACATCTCTTGGCGCTTGAAAGAAAGCTGAAGAAAATGCTGG | |
| | GCCCCTCTGCTGTAGAGATAGGGAATGGATGCTTTGAAACCAAACACAAGT | |
| | GCAACCAGACCTGCCTCGACAGAATAGCTGCTGGTACCTTTGATGCAGGAG | |
| | AATTTTCTCTCCCCACCTTTGATTCACTGAATATTACTGCTGCATCTTTAAAT | |
| | GACGACGGATTGGACAATCATACTATACTGCTTTACTACTCAACTGCTGCCT | |
| | CCAGTTTGGCTGTAACACTGATGATAGCTATCTTTGTTGTTTATATGGTCTCC | |
| | AGAGACAATGTTTCTTGCTCCATTTGTCTATAAGGAAAGTTAAGCCCTGTATT | |
| | TTCCTTTATTGTAGTGCTTGTTTGCTTGTTGTCATTACAAAGAAACGTTATTG | |
| | AAAAATGGAAGCGGAGCCACGAACTTCTCTCTGTTAAAGCAAGCAGGAGAT | |
| | GTTGAAGAAAACCCCGGGCCTTTCGGAGCTATTGCTGGTTTCTTGGAAGGA | |
| | GGATGGGAAGGAATGATTGCAGGTTGGCACGGATACACATCTCATGGAGCA | |
| | CATGGAGTAGCAGTGGCAGCAGACCTTAAGAGTACCCATGATGACTCGAGC | |
| | TGGTACTGCATGCACGCAATGCTAGCTGCCCCTTTCCCGTCCTGGGTACCC | |
| | CGAGTCTCCCCCGACCTCGGGTCCCAGGTATGCTCCCACCTCCACCTGCC | |
| | CCACTCACCACCTCTGCTAGTTCCAGACACCTCCCAAGCACGCAGCAATGC | |
| | AGCTCAAAACGCTTAGCCTAGCCACACCCCCACGGGAAACAGCAGTGATTA | |
| | ACCTTTAGCAATAAACGAAAGTTTAACTAAGCTATACTAACCCCAGGGTTGG | |
| | TCAATTTCGTGCCAGCCACACCCTGGAGCTAGCAAAAAAAA | |
| Pan-CoV- | CTCGACATTGATTATTGACTAGTTATTAATAGTAATCAATTACGGGGTCATTA | 33 |
| Flu | GTTCATAGCCCATATATGGAGTTCCGCGTTACATAACTTACGGTAAATGGCC | |
| Vaccine | CGCCTGGCTGACCGCCCAACGACCCCCGCCCATTGACGTCAATAATGACGT | |
| #13 CoV- | ATGTTCCCATAGTAACGCCAATAGGGACTTTCCATTGACGTCAATGGGTGGA | |
| Wuhan | GTATTTACGGTAAACTGCCCACTTGGCAGTACATCAAGTGTATCATATGCCA | |
| Spike | AGTACGCCCCCTATTGACGTCAATGACGGTAAATGGCCCGCCTGGCATTAT | |
| (HexaPro)- | GCCCAGTACATGACCTTATGGGACTTTCCTACTTGGCAGTACATCTACGTAT | |
| Influenza | TAGTCATCGCTATTACCATGGTCGAGGTGAGCCCCACGTTCTGCTTCACTCT | |
| (HA) | CCCCATCTCCCCCCCCTCCCCACCCCCAATTTTGTATTTATTTATTTTTTAAT | |
| | TATTTTGTGCAGCGATGGGGGCGGGGGGGGGGGGGGGGCGCGCGCCAG | |
| | GCGGGGCGGGGCGGGGCGAGGGGCGGGGCGGGGCGAGGCGGAGAGGT | |
| | GCGGCGGCAGCCAATCAGAGCGGCGCGCTCCGAAAGTTTCCTTTTATGGC | |
| | GAGGCGGCGGCGGCGGCGGCCCTATAAAAAGCGAAGCGCGCGGCGGGC | |
| | GGGAGTCGCTGCGCGCTGCCTTCGCCCCGTGCCCCGCTCCGCCGCCGCC | |
| | TCGCGCCGCCCGCCCCGGCTCTGACTGACCGCGTTACTCCCACAGGTGAG | |
| | CGGGCGGGACGGCCCTTCTCCTCCGGGCTGTAATTAGCGCTTGGTTTAATG | |
| | ACGGCTTGTTTCTTTTCTGTGGCTGCGTGAAAGCCTTGAGGGGCTCCGGGA | |
| | GGGCCCTTTGTGCGGGGGGAGCGGCTCGGGGGGTGCGTGCGTGTGTGTG | |
| | TGCGTGGGGAGCGCCGCGTGCGGCTCCGCGCTGCCCGGCGGCTGTGAGC | |
| | GCTGCGGGCGCGGCGCGGGGCTTTGTGCGCTCCGCAGTGTGCGCGAGGG | |
| | GAGCGCGGCCGGGGGGGGTGCCCCGCGGTGCGGGGGGGGCTGCGAGGG | |
| | GAACAAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCCCGAGT | |
| | TGCTGAGCACGGCCCGGCTTCGGGTGCGGGGCTCCGTACGGGGCGTGGC | |
| | GCGGGGCTCGCCGTGCCGGGCGGGGGGTGGCGGCAGGTGGGGGTGCCG | |
| | GGCGGGGCGGGGCCGCCTCGGGCCGGGGAGGGCTCGGGGGAGGGGCG | |
| | CGGCGGCCCCCGGAGCGCCGGCGGCTGTCGAGGCGCGGCGAGCCGCAG | |
| | CCATTGCCTTTTATGGTAATCGTGCGAGAGGGCGCAGGGACTTCCTTTGTC | |
| | CCAAATCTGTGCGGAGCCGAAATCTGGGAGGCGCCGCCGCACCCCCTCTA | |
| | GCGGGCGCGGGGCGAAGCGGTGCGGCGCCGGCAGGAAGGAAATGGGCG | |
| | GGGAGGGCCTTCGTGCGTCGCCGCGCCGCCGTCCCCTTCTCCCTCTCCAG | |
| | CCTCGGGGCTGTCCGCGGGGGGACGGCTGCCTTCGGGGGGGACGGGGC | |
| | AGGGGGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGTGCTG | |
| | GTTATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTTCCT | |
| | GGTGCTGCTGCCCCTGGTGAGCAGCCAGTGCGTGAACCTGACCACCAGGA | |
| | CCCAGCTGCCCCCCGCCTACACCAACAGCTTCACCAGGGGCGTGTACTAC | |
| | CCCGACAAGGTGTTCAGGAGCAGCGTGCTGCACAGCACCCAGGACCTGTT | |
| | CCTGCCCTTCTTCAGCAACGTGACCTGGTTCCACGCCATCCACGTGAGCGG | |
| | CACCAACGGCACCAAGAGGTTCGACAACCCCGTGCTGCCCTTCAACGACG | |
| | GCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAAC | |
| | GCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAG | |
| | TTCAGGGTGTACAGCAGCGCCAACAACTGCACCTTCGAGTACGTGAGCCAG | |
| | CCCTTCCTGATGGACCTGGAGGGCAAGCAGGGCAACTTCAAGAACCTGAG | |
| | GGAGTTCGTGTTCAAGAACATCGACGGCTACTTCAAGATCTACAGCAAGCA | |
| | CACCCCCATCAACCTGGTGAGGGACCTGCCCCAGGGCTTCAGCGCCCTGG | |
| | AGCCCCTGGTGGACCTGCCCATCGGCATCAACATCACCAGGTTCCAGACCC | |
| | TGCTGGCCCTGCACAGGAGCTACCTGACCCCCGGCGACAGCAGCAGCGGC | |
| | TGGACCGCCGGCGCCGCCGCCTACTACGTGGGCTACCTGCAGCCCAGGAC | |
| | CTTCCTGCTGAAGTACAACGAGAACGGCACCATCACCGACGCCGTGGACTG | |
| | CGCCCTGGACCCCCTGAGCGAGACCAAGTGCACCCTGAAGAGCTTCACCG | |
| | TGGAGAAGGGCATCTACCAGACCAGCAACTTCAGGGTGCAGCCCACCGAG | |
| | AGCATCGTGAGGTTCCCCAACATCACCAACCTGTGCCCCTTCGGCGAGGTG | |
| | TTCAACGCCACCAGGTTCGCCAGCGTGTACGCCTGGAACAGGAAGAGGAT | |
| | CAGCAACTGCGTGGCCGACTACAGCGTGCTGTACAACAGCGCCAGCTTCA | |
| | GCACCTTCAAGTGCTACGGCGTGAGCCCCACCAAGCTGAACGACCTGTGCT | |
| | TCACCAACGTGTACGCCGACAGCTTCGTGATCAGGGGCGACGAGGTGAGG | |
| | CAGATCGCCCCCGGCCAGACCGGCAAGATCGCCGACTACAACTACAAGCT | |
| | GCCCGACGACTTCACCGGCTGCGTGATCGCCTGGAACAGCAACAACCTGG | |
| | ACAGCAAGGTGGGCGGCAACTACAACTACCTGTACAGGCTGTTCAGGAAGA | |
| | GCAACCTGAAGCCCTTCGAGAGGGACATCAGCACCGAGATCTACCAGGCC | |
| | GGCAGCACCCCCTGCAACGGCGTGGAGGGCTTCAACTGCTACTTCCCCCT | |
| | GCAGAGCTACGGCTTCCAGCCCACCAACGGCGTGGGCTACCAGCCCTACA | |
| | GGGTGGTGGTGCTGAGCTTCGAGCTGCTGCACGCCCCCGCCACCGTGTGC | |
| | GGCCCCAAGAAGAGCACCAACCTGGTGAAGAACAAGTGCGTGAACTTCAAC | |
| | TTCAACGGCCTGACCGGCACCGGCGTGCTGACCGAGAGCAACAAGAAGTT | |
| | CCTGCCCTTCCAGCAGTTCGGCAGGGACATCGCCGACACCACCGACGCCG | |
| | TGAGGGACCCCCAGACCCTGGAGATCCTGGACATCACCCCCTGCAGCTTC | |
| | GGCGGCGTGAGCGTGATCACCCCCGGCACCAACACCAGCAACCAGGTGGC | |
| | CGTGCTGTACCAGGACGTGAACTGCACCGAGGTGCCCGTGGCCATCCACG | |
| | CCGACCAGCTGACCCCCACCTGGAGGGTGTACAGCACCGGCAGCAACGTG | |
| | TTCCAGACCAGGGCCGGCTGCCTGATCGGCGCCGAGCACGTGAACAACAG | |
| | CTACGAGTGCGACATCCCCATCGGCGCCGGCATCTGCGCCAGCTACCAGA | |
| | CCCAGACCAACAGCCCCGGCAGCGCCAGCAGCGTGGCCAGCCAGAGCAT | |
| | CATCGCCTACACCATGAGCCTGGGCGCCGAGAACAGCGTGGCCTACAGCA | |
| | ACAACAGCATCGCCATCCCCACCAACTTCACCATCAGCGTGACCACCGAGA | |
| | TCCTGCCCGTGAGCATGACCAAGACCAGCGTGGACTGCACCATGTACATCT | |
| | GCGGCGACAGCACCGAGTGCAGCAACCTGCTGCTGCAGTACGGCAGCTTC | |
| | TGCACCCAGCTGAACAGGGCCCTGACCGGCATCGCCGTGGAGCAGGACAA | |
| | GAACACCCAGGAGGTGTTCGCCCAGGTGAAGCAGATCTACAAGACCCCCC | |
| | CCATCAAGGACTTCGGCGGCTTCAACTTCAGCCAGATCCTGCCCGACCCCA | |
| | GCAAGCCCAGCAAGAGGAGCCCCATCGAGGACCTGCTGTTCAACAAGGTG | |
| | ACCCTGGCCGACGCCGGCTTCATCAAGCAGTACGGCGACTGCCTGGGCGA | |
| | CATCGCCGCCAGGGACCTGATCTGCGCCCAGAAGTTCAACGGCCTGACCG | |
| | TGCTGCCCCCCCTGCTGACCGACGAGATGATCGCCCAGTACACCAGCGCC | |
| | CTGCTGGCCGGCACCATCACCAGCGGCTGGACCTTCGGCGCCGGCCCCG | |
| | CCCTGCAGATCCCCTTCCCCATGCAGATGGCCTACAGGTTCAACGGCATCG | |
| | GCGTGACCCAGAACGTGCTGTACGAGAACCAGAAGCTGATCGCCAACCAG | |
| | TTCAACAGCGCCATCGGCAAGATCCAGGACAGCCTGAGCAGCACCCCCAG | |
| | CGCCCTGGGCAAGCTGCAGGACGTGGTGAACCAGAACGCCCAGGCCCTGA | |
| | ACACCCTGGTGAAGCAGCTGAGCAGCAACTTCGGCGCCATCAGCAGCGTG | |
| | CTGAACGACATCCTGAGCAGGCTGGACCCCCCCGAGGCCGAGGTGCAGAT | |
| | CGACAGGCTGATCACCGGCAGGCTGCAGAGCCTGCAGACCTACGTGACCC | |
| | AGCAGCTGATCAGGGCCGCCGAGATCAGGGCCAGCGCCAACCTGGCCGC | |
| | CACCAAGATGAGCGAGTGCGTGCTGGGCCAGAGCAAGAGGGTGGACTTCT | |
| | GCGGCAAGGGCTACCACCTGATGAGCTTCCCCCAGAGCGCCCCCCACGGC | |
| | GTGGTGTTCCTGCACGTGACCTACGTGCCCGCCCAGGAGAAGAACTTCACC | |
| | ACCGCCCCCGCCATCTGCCACGACGGCAAGGCCCACTTCCCCAGGGAGGG | |
| | CGTGTTCGTGAGCAACGGCACCCACTGGTTCGTGACCCAGAGGAACTTCTA | |
| | CGAGCCCCAGATCATCACCACCGACAACACCTTCGTGAGCGGCAACTGCG | |
| | ACGTGGTGATCGGCATCGTGAACAACACCGTGTACGACCCCCTGCAGCCC | |
| | GAGCTGGACAGCTTCAAGGAGGAGCTGGACAAGTACTTCAAGAACCACACC | |
| | AGCCCCGACGTGGACCTGGGCGACATCAGCGGCATCAACGCCAGCGTGGT | |
| | GAACATCCAGAAGGAGATCGACAGGCTGAACGAGGTGGCCAAGAACCTGA | |
| | ACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATC | |
| | GTGATGGTGACCATCATGCTGTGCTGCATGACCAGCTGCTGCAGCTGCCTG | |
| | AAGGGCTGCTGCAGCTGCGGCAGCTGCTGCAAGTTCGACGAGGACGACAG | |
| | CGAGCCCGTGCTGAAGGGCGTGAAGCTGCACTACACCGGAAGCGGAGCCA | |
| | CGAACTTCTCTCTGTTAAAGCAAGCAGGAGATGTTGAAGAAAACCCCGGGC | |
| | CTATGAAGGCAATACTAGTAGTTCTGCTATATACATTTGCAACCGCAAATGC | |
| | AGACACATTATGTATAGGTTATCATGCGAACAACTCAACAGACACTGTAGAC | |
| | ACAGTACTAGAAAAGAATGTAACAGTAACACACTCTGTTAACCTTCTAGAAG | |
| | ACAAGCATAACGGGAAACTATGCAAACTAAGAGGGGTAGCCCCATTGCATT | |
| | TGGGTAAATGTAACATTGCTGGCTGGATCCTGGGAAATCCAGAGTGTGAAT | |
| | CACTCTCCACAGCAAGCTCATGGTCCTACATTGTGGAAACATCTAGTTCAGA | |
| | CAATGGAACGTGTTACCCAGGAGATTTCATCGATTATGAGGAGCTAAGAGA | |
| | GCAATTGAGCTCAGTGTCATCATTTGAAAGGTTTGAGATATTCCCCAAGACA | |
| | AGTTCATGGCCCAATCATGACTCGAACAAAGGTGTAACGACAGCATGTCCT | |
| | CATGCTGGAGCAAAAAGCTTCTACAAAAATTTAATATGGCTAGTTAAAAAAG | |
| | GAAATTCATACCCAAAGCTCAGCAAATCCTACATTAATGATAAAGGGAAAGA | |
| | AGTCCTCGTGCTATGGGGCATTCACCATCCACCTACTAGTGCTGACCAACA | |
| | AAGTCTCTATCAGAATGCAGATGCATATGTTTTTGTGGGGACATCAAGATAC | |
| | AGCAAGAAGTTCAAGCCGGAAATAGCAATAAGGCCCAAAGTGAGGGATCAA | |
| | GAAGGGAGAATGAACTATTACTGGACACTAGTAGAGCCGGGAGACAAAATA | |
| | ACATTCGAAGCAACTGGAAATCTAGTGGTACCGAGATATGCATTCGCAATG | |
| | GAAAGAAATGCTGGATCTGGTATTATCATTTCAGATACACCAGTCCACGATT | |
| | GCAATACAACTTGTCAGACACCCAAGGGTGCTATAAACACCAGCCTCCCATT | |
| | TCAGAACATACATCCGATCACAATTGGAAAATGTCCAAAATATGTAAAAAGC | |
| | ACAAAATTGAGACTGGCCACAGGATTGAGGAATGTCCCGTCCATTCAATCTA | |
| | GAGGCCTATTTGGGGCCATTGCCGGTTTCATTGAAGGGGGGTGGACAGGG | |
| | ATGGTAGATGGATGGTACGGTTATCACCATCAAAATGAGCAGGGGTCAGGA | |
| | TATGCAGCCGACCTGAAGAGCACACAGAATGCCATTGACGAGATTACTAAC | |
| | AAAGTAAACTCTGTTATTGAAAAAATGAATACACAGTTCACAGCAGTAGGTA | |
| | AAGAGTTCAACCACCTGGAAAAAAGAATAGAGAATTTAAATAAAAAAGTTGA | |
| | TGATGGTTTCCTGGACATTTGGACTTACAATGCCGAACTGTTGGTTCTATTG | |
| | GAAAATGAAAGAACTTTGGACTACCACGATTCAAAGGTGAAGAACTTATATG | |
| | AAAAGGTAAGAAGCCAGTTAAAAAACAATGCCAAGGAAATTGGAAACGGCT | |
| | GCTTTGAATTTTACCACAAATGTGATAACACGTGCATGGAAAGTGTCAAAAA | |
| | TGGGACTTATGACTACCCAAAATACTCAGAGGAAGCAAAATTAAACAGAGAA | |
| | GAAATAGATGGGGTAAAGCTGGAATCAACAAGGATTTACCAGATTTTGGCG | |
| | ATCTATTCAACCGTCGCCAGTTCATTGGTACTGGTAGTCTCCCTGGGGGCA | |
| | ATCAGTTTCTGGATGTGCTCTAATGGGTCTCTACAGTGTAGAATATGTATTTA | |
| | AGGAAGCGGAGCCACGAACTTCTCTCTGTTAAAGCAAGCAGGAGATGTTGA | |
| | AGAAAACCCCGGGCCTAGCAAAAGCAGGGGATAATTCTATTAACCATGAAG | |
| | ACTATCATTGCTTTGAGCTACATTCTATGTCTGGTTTTCGCTCAAAAACTTCC | |
| | TGGAAATGACAATAGCACTGCAACGCTGTGCCTTGGGCACCATGCAGTACC | |
| | AAACGGAACGATAGTGAAAACAATCACGAATGACCAAATTGAAGTTACTAAT | |
| | GCTACTGAGCTGGTTCAGAATTCCTCAATAGGTGAAATATGCGACAGTCCTC | |
| | ATCAGATCCTTGATGGAGAAAACTGCACACTAATAGATGCTCTATTGGGAGA | |
| | CCCTCAGTGTGATGGCTTTCAAAATAAGAAATGGGACCTTTTTGTTGAACGA | |
| | AGCAAAGCCTACAGCAACTGTTACCCTTATGATGTGCCGGATTATGCCTCCC | |
| | TTAGGTCACTAGTTGCCTCATCCGGCACACTGGAGTTTAACAATGAAAGCTT | |
| | CAATTGGGCTGGAGTCACTCAAAACGGAACAAGTTCTGCTTGCATAAGGGG | |
| | ATCTAATAGTAGTTTCTTTAGTAGATTAAATTGGTTGACCCACTTAAACTTCA | |
| | AGTACCCAGCATTGAACGTGACTATGCCAAACAATGAACAATTTGACAAATT | |
| | GTACATTTGGGGGGTTCACCACCCGGGTACGGACAAGGACCAAATCTTCCT | |
| | GTATGCTCAATCATCAGGAAGAATCACAGTATCTACCAAAAGAAGCCAACAA | |
| | GCTGTAATCCCGAATATCGGATCTAGACCCAGAATAAGGAATATCCCTAGCA | |
| | GAATAAGCATCTATTGGACAATAGTAAAACCGGGAGACATACTTTTGATTAA | |
| | CAGCACAGGGAATCTAATTGCTCCTAGGGGTTACTTCAAAATACGAAGTGG | |
| | GAAAAGCTCAATAATGAGATCAGATGCACCCATTGGCAAATGCAAGTCTGAA | |
| | TGCATCACTCCAAATGGAAGCATTCCCAATGACAAACCATTCCAAAATGTAA | |
| | ACAGGATCACATACGGGGCCTGTCCCAGATATGTTAAGCAAAGCACTCTGA | |
| | AATTGGCAACAGGAATGCGAAATGTACCAGAGAAACAAACTAGAGGCATATT | |
| | TGGCGCAATAGCGGGTTTCATAGAAAATGGTTGGGAGGGAATGGTGGATG | |
| | GTTGGTACGGTTTCAGGCATCAAAATTCTGAGGGAAGAGGACAAGCAGCAG | |
| | ATCTCAAAAGCACTCAAGCAGCAATCGATCAAATCAATGGGAAGCTGAATCG | |
| | ATTGATCGGGAAAACCAACGAGAAATTCCATCAGATTGAGAAAGAATTCTCA | |
| | GAAGTAGAAGGGAGAATTCAGGACCTTGAGAAATATGTTGAGGACACAAAA | |
| | ATAGATCTCTGGTCATACAACGCAGAGCTTCTTGTTGCCCTGGAAAACCAAC | |
| | ATACAATTGATCTAACTGACTCAGAAATGAACAAACTGTTTGAAAAAACAAAG | |
| | AAGCAACTGAGGGAAAATGCTGAGGATATGGGCAATGGTTGTTTCAAAATAT | |
| | ACCACAAATGTGACAATGCCTGCATAGGATCAATCAGAAATGGAACTTATGA | |
| | CCACGATGTATACAGGGATGAAGCATTAAACAACCGGTTCCAGATCAAGGG | |
| | AGTTGAGCTGAAGTCAGGGTACAAAGATTGGATCCTATGGATTTCCTTTGCC | |
| | ATATCATGTTTTTTGCTTTGTGTTGCTTTGTTGGGGTTCATCATGTGGGCCTG | |
| | CCAAAAGGGCAACATTAGGTGCAACATTTGCATTTGAGTGCATTAATTAAAA | |
| | ACACCCTTGTTTCTACTGGAAGCGGAGCCACGAACTTCTCTCTGTTAAAGCA | |
| | AGCAGGAGATGTTGAAGAAAACCCCGGGCCTATTTTCTAATATCCACAAAAT | |
| | GAAGGCAATAATTGTACTACTCATGGTAGTAACATCCAATGCAGATCGAATC | |
| | TGCACTGGGATAACATCGTCAAACTCACCACATGTCGTCAAAACTGCTACTC | |
| | AAGGGGAGGTCAACGTGACCGGTGTAATACCACTGACAACAACACCCACCA | |
| | AATCTCATTTTGCAAATCTCAAAGGAACAGAAACCAGGGGGAAACTATGCCC | |
| | AAAATGCCTCAACTGCACAGATCTGGATGTAGCCTTGGGCAGACCAAAATG | |
| | CACAGGGAAAATACCCTCTGCAAGGGTTTCAATACTCCATGAAGTCAGACCT | |
| | GTTACATCTGGGTGCTTTCCTATAATGCACGATAGAACAAAAATTAGACAGC | |
| | TGCCTAACCTTCTCCGAGGATACGAACATGTCAGGTTATCAACTCACAACGT | |
| | TATCAATGCAAAAGATGCACCAGGAAGACCCTACGAAATTGGAACCTCAGG | |
| | GTCTTGCCCTAACATTACCAATGGAAACGGATTCTTCGCAACAATGGCTTGG | |
| | GCCGTCCCAAAAAACAAAACAGCAACAAATCCATTAACAATAGAAGTACCAT | |
| | ACATTTGTACAGAAGGAGAAGACCAAATTACCGTTTGGGGGTTCCACTCTGA | |
| | CAACGAGACCCAAATGGCAAAGCTCTATGGGGACTCAAAGCCCCAGAAGTT | |
| | CACCTCATCTGCCAACGGAGTGACCACACATTACGTTTCACAGATTGGTGG | |
| | CTTCCCAAATCAAACAGAAGACGGAGGACTACCACAAAGTGGCAGAATTGT | |
| | TGTTGATTACATGGTGCAGAAATCTGGAAAAACAGGAACAATTACCTATCAA | |
| | AGAGGTATTTTATTGCCTCAAAAAGTGTGGTGCGCAAGTGGCAGGAGCAAG | |
| | GTAATAAAAGGATCCTTGCCCTTAATTGGAGAAGCAGATTGCCTCCATGAAA | |
| | AATACGGTGGATTAAACAAAAGCAAGCCTTACTACACAGGGGAACATGCAA | |
| | AGGCCATAGGAAATTGCCCAATATGGGTGAAAACACCCTTGAAGCTGGCCA | |
| | ATGGAACCAAATATAGACCCCCTGCAAAACTATTAAAGGAAAGAGGTTTCTT | |
| | CGGAGCCATTGCTGGTTTCTTAGAGGGAGGATGGGAAGGAATGATTGCAG | |
| | GTTGGCACGGATACACATCCCATGGGGCACATGGAGTAGCGGTGGCAGCT | |
| | GACCTTAAGAGCACTCAAGAGGCCATAAACAAGATAACAAAAAATCTCAACT | |
| | CTTTGAGTGAGCTGGAAGTAAAGAATCTTCAAAGACTAAGCGGTGCCATGG | |
| | ATGAACTCCACAACGAAATACTAGAACTAGATGAGAAAGTGGATGATCTCAG | |
| | AGCTGACACAATAAGCTCACAAATAGAACTCGCAGTCCTGCTTTCCAATGAA | |
| | GGAATAATAAACAGTGAAGATGAACATCTCTTGGCGCTTGAAAGAAAGCTGA | |
| | AGAAAATGCTGGGCCCCTCTGCTGTAGAGATAGGGAATGGATGCTTTGAAA | |
| | CCAAACACAAGTGCAACCAGACCTGCCTCGACAGAATAGCTGCTGGTACCT | |
| | TTGATGCAGGAGAATTTTCTCTCCCCACCTTTGATTCACTGAATATTACTGCT | |
| | GCATCTTTAAATGACGACGGATTGGACAATCATACTATACTGCTTTACTACTC | |
| | AACTGCTGCCTCCAGTTTGGCTGTAACACTGATGATAGCTATCTTTGTTGTT | |
| | TATATGGTCTCCAGAGACAATGTTTCTTGCTCCATTTGTCTATAAGGAAAGTT | |
| | AAGCCCTGTATTTTCCTTTATTGTAGTGCTTGTTTGCTTGTTGTCATTACAAA | |
| | GAAACGTTATTGAAAAATGGAAGCGGAGCCACGAACTTCTCTCTGTTAAAGC | |
| | AAGCAGGAGATGTTGAAGAAAACCCCGGGCCTTTCGGAGCTATTGCTGGTT | |
| | TCTTGGAAGGAGGATGGGAAGGAATGATTGCAGGTTGGCACGGATACACAT | |
| | CTCATGGAGCACATGGAGTAGCAGTGGCAGCAGACCTTAAGAGTACCCATG | |
| | ATGACTCGAGCTGGTACTGCATGCACGCAATGCTAGCTGCCCCTTTCCCGT | |
| | CCTGGGTACCCCGAGTCTCCCCCGACCTCGGGTCCCAGGTATGCTCCCAC | |
| | CTCCACCTGCCCCACTCACCACCTCTGCTAGTTCCAGACACCTCCCAAGCA | |
| | CGCAGCAATGCAGCTCAAAACGCTTAGCCTAGCCACACCCCCACGGGAAAC | |
| | AGCAGTGATTAACCTTTAGCAATAAACGAAAGTTTAACTAAGCTATACTAACC | |
| | CCAGGGTTGGTCAATTTCGTGCCAGCCACACCCTGGAGCTAGCAAAAAAAA | |
| Pan-CoV- | CTCGACATTGATTATTGACTAGTTATTAATAGTAATCAATTACGGGGTCATTA | 34 |
| Flu | GTTCATAGCCCATATATGGAGTTCCGCGTTACATAACTTACGGTAAATGGCC | |
| Vaccine | CGCCTGGCTGACCGCCCAACGACCCCCGCCCATTGACGTCAATAATGACGT | |
| #14 CoV- | ATGTTCCCATAGTAACGCCAATAGGGACTTTCCATTGACGTCAATGGGTGGA | |
| Wuhan | GTATTTACGGTAAACTGCCCACTTGGCAGTACATCAAGTGTATCATATGCCA | |
| Spike | AGTACGCCCCCTATTGACGTCAATGACGGTAAATGGCCCGCCTGGCATTAT | |
| (2 proline | GCCCAGTACATGACCTTATGGGACTTTCCTACTTGGCAGTACATCTACGTAT | |
| subst.)- | TAGTCATCGCTATTACCATGGTCGAGGTGAGCCCCACGTTCTGCTTCACTCT | |
| Influenza | CCCCATCTCCCCCCCCTCCCCACCCCCAATTTTGTATTTATTTATTTTTTAAT | |
| (HA) | TATTTTGTGCAGCGATGGGGGCGGGGGGGGGGGGGGGGCGCGCGCCAG | |
| | GCGGGGCGGGGCGGGGCGAGGGGCGGGGCGGGGCGAGGCGGAGAGGT | |
| | GCGGCGGCAGCCAATCAGAGCGGCGCGCTCCGAAAGTTTCCTTTTATGGC | |
| | GAGGCGGCGGCGGCGGCGGCCCTATAAAAAGCGAAGCGCGCGGGGGC | |
| | GGGAGTCGCTGCGCGCTGCCTTCGCCCCGTGCCCCGCTCCGCCGCCGCC | |
| | TCGCGCCGCCCGCCCCGGCTCTGACTGACCGCGTTACTCCCACAGGTGAG | |
| | CGGGCGGGACGGCCCTTCTCCTCCGGGCTGTAATTAGCGCTTGGTTTAATG | |
| | ACGGCTTGTTTCTTTTCTGTGGCTGCGTGAAAGCCTTGAGGGGCTCCGGGA | |
| | GGGCCCTTTGTGCGGGGGGAGCGGCTCGGGGGGTGCGTGCGTGTGTGTG | |
| | TGCGTGGGGAGCGCCGCGTGCGGCTCCGCGCTGCCCGGCGGCTGTGAGC | |
| | GCTGCGGGCGCGGCGCGGGGCTTTGTGCGCTCCGCAGTGTGCGCGAGGG | |
| | GAGCGCGGCCGGGGGGGGTGCCCCGCGGTGCGGGGGGGGCTGCGAGGG | |
| | GAACAAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCCCGAGT | |
| | TGCTGAGCACGGCCCGGCTTCGGGTGCGGGGCTCCGTACGGGGCGTGGC | |
| | GCGGGGCTCGCCGTGCCGGGGGGGGGGGGCGGCAGGTGGGGGTGCCG | |
| | GGCGGGGGGGGGCCGCCTCGGGCCGGGGAGGGCTCGGGGGAGGGGCG | |
| | CGGCGGCCCCCGGAGCGCCGGCGGCTGTCGAGGCGCGGCGAGCCGCAG | |
| | CCATTGCCTTTTATGGTAATCGTGCGAGAGGGCGCAGGGACTTCCTTTGTC | |
| | CCAAATCTGTGCGGAGCCGAAATCTGGGAGGCGCCGCCGCACCCCCTCTA | |
| | GCGGGCGCGGGGCGAAGCGGTGCGGCGCCGGCAGGAAGGAAATGGGCG | |
| | GGGAGGGCCTTCGTGCGTCGCCGCGCCGCCGTCCCCTTCTCCCTCTCCAG | |
| | CCTCGGGGCTGTCCGCGGGGGGACGGCTGCCTTCGGGGGGGACGGGGC | |
| | AGGGGGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGTGCTG | |
| | GTTATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTTCCT | |
| | GGTGCTGCTGCCCCTGGTGAGCAGCCAGTGCGTGAACCTGACCACCAGGA | |
| | CCCAGCTGCCCCCCGCCTACACCAACAGCTTCACCAGGGGCGTGTACTAC | |
| | CCCGACAAGGTGTTCAGGAGCAGCGTGCTGCACAGCACCCAGGACCTGTT | |
| | CCTGCCCTTCTTCAGCAACGTGACCTGGTTCCACGCCATCCACGTGAGCGG | |
| | CACCAACGGCACCAAGAGGTTCGACAACCCCGTGCTGCCCTTCAACGACG | |
| | GCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAAC | |
| | GCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAG | |
| | TTCAGGGTGTACAGCAGCGCCAACAACTGCACCTTCGAGTACGTGAGCCAG | |
| | CCCTTCCTGATGGACCTGGAGGGCAAGCAGGGCAACTTCAAGAACCTGAG | |
| | GGAGTTCGTGTTCAAGAACATCGACGGCTACTTCAAGATCTACAGCAAGCA | |
| | CACCCCCATCAACCTGGTGAGGGACCTGCCCCAGGGCTTCAGCGCCCTGG | |
| | AGCCCCTGGTGGACCTGCCCATCGGCATCAACATCACCAGGTTCCAGACCC | |
| | TGCTGGCCCTGCACAGGAGCTACCTGACCCCCGGCGACAGCAGCAGCGGC | |
| | TGGACCGCCGGCGCCGCCGCCTACTACGTGGGCTACCTGCAGCCCAGGAC | |
| | CTTCCTGCTGAAGTACAACGAGAACGGCACCATCACCGACGCCGTGGACTG | |
| | CGCCCTGGACCCCCTGAGCGAGACCAAGTGCACCCTGAAGAGCTTCACCG | |
| | TGGAGAAGGGCATCTACCAGACCAGCAACTTCAGGGTGCAGCCCACCGAG | |
| | AGCATCGTGAGGTTCCCCAACATCACCAACCTGTGCCCCTTCGGCGAGGTG | |
| | TTCAACGCCACCAGGTTCGCCAGCGTGTACGCCTGGAACAGGAAGAGGAT | |
| | CAGCAACTGCGTGGCCGACTACAGCGTGCTGTACAACAGCGCCAGCTTCA | |
| | GCACCTTCAAGTGCTACGGCGTGAGCCCCACCAAGCTGAACGACCTGTGCT | |
| | TCACCAACGTGTACGCCGACAGCTTCGTGATCAGGGGCGACGAGGTGAGG | |
| | CAGATCGCCCCCGGCCAGACCGGCAAGATCGCCGACTACAACTACAAGCT | |
| | GCCCGACGACTTCACCGGCTGCGTGATCGCCTGGAACAGCAACAACCTGG | |
| | ACAGCAAGGTGGGCGGCAACTACAACTACCTGTACAGGCTGTTCAGGAAGA | |
| | GCAACCTGAAGCCCTTCGAGAGGGACATCAGCACCGAGATCTACCAGGCC | |
| | GGCAGCACCCCCTGCAACGGCGTGGAGGGCTTCAACTGCTACTTCCCCCT | |
| | GCAGAGCTACGGCTTCCAGCCCACCAACGGCGTGGGCTACCAGCCCTACA | |
| | GGGTGGTGGTGCTGAGCTTCGAGCTGCTGCACGCCCCCGCCACCGTGTGC | |
| | GGCCCCAAGAAGAGCACCAACCTGGTGAAGAACAAGTGCGTGAACTTCAAC | |
| | TTCAACGGCCTGACCGGCACCGGCGTGCTGACCGAGAGCAACAAGAAGTT | |
| | CCTGCCCTTCCAGCAGTTCGGCAGGGACATCGCCGACACCACCGACGCCG | |
| | TGAGGGACCCCCAGACCCTGGAGATCCTGGACATCACCCCCTGCAGCTTC | |
| | GGCGGCGTGAGCGTGATCACCCCCGGCACCAACACCAGCAACCAGGTGGC | |
| | CGTGCTGTACCAGGACGTGAACTGCACCGAGGTGCCCGTGGCCATCCACG | |
| | CCGACCAGCTGACCCCCACCTGGAGGGTGTACAGCACCGGCAGCAACGTG | |
| | TTCCAGACCAGGGCCGGCTGCCTGATCGGCGCCGAGCACGTGAACAACAG | |
| | CTACGAGTGCGACATCCCCATCGGCGCCGGCATCTGCGCCAGCTACCAGA | |
| | CCCAGACCAACAGCCCCAGGAGGGCCAGGAGCGTGGCCAGCCAGAGCAT | |
| | CATCGCCTACACCATGAGCCTGGGCGCCGAGAACAGCGTGGCCTACAGCA | |
| | ACAACAGCATCGCCATCCCCACCAACTTCACCATCAGCGTGACCACCGAGA | |
| | TCCTGCCCGTGAGCATGACCAAGACCAGCGTGGACTGCACCATGTACATCT | |
| | GCGGCGACAGCACCGAGTGCAGCAACCTGCTGCTGCAGTACGGCAGCTTC | |
| | TGCACCCAGCTGAACAGGGCCCTGACCGGCATCGCCGTGGAGCAGGACAA | |
| | GAACACCCAGGAGGTGTTCGCCCAGGTGAAGCAGATCTACAAGACCCCCC | |
| | CCATCAAGGACTTCGGCGGCTTCAACTTCAGCCAGATCCTGCCCGACCCCA | |
| | GCAAGCCCAGCAAGAGGAGCTTCATCGAGGACCTGCTGTTCAACAAGGTGA | |
| | CCCTGGCCGACGCCGGCTTCATCAAGCAGTACGGCGACTGCCTGGGCGAC | |
| | ATCGCCGCCAGGGACCTGATCTGCGCCCAGAAGTTCAACGGCCTGACCGT | |
| | GCTGCCCCCCCTGCTGACCGACGAGATGATCGCCCAGTACACCAGCGCCC | |
| | TGCTGGCCGGCACCATCACCAGCGGCTGGACCTTCGGCGCCGGCGCCGC | |
| | CCTGCAGATCCCCTTCGCCATGCAGATGGCCTACAGGTTCAACGGCATCGG | |
| | CGTGACCCAGAACGTGCTGTACGAGAACCAGAAGCTGATCGCCAACCAGTT | |
| | CAACAGCGCCATCGGCAAGATCCAGGACAGCCTGAGCAGCACCGCCAGCG | |
| | CCCTGGGCAAGCTGCAGGACGTGGTGAACCAGAACGCCCAGGCCCTGAAC | |
| | ACCCTGGTGAAGCAGCTGAGCAGCAACTTCGGCGCCATCAGCAGCGTGCT | |
| | GAACGACATCCTGAGCAGGCTGGACCCCCCCGAGGCCGAGGTGCAGATCG | |
| | ACAGGCTGATCACCGGCAGGCTGCAGAGCCTGCAGACCTACGTGACCCAG | |
| | CAGCTGATCAGGGCCGCCGAGATCAGGGCCAGCGCCAACCTGGCCGCCA | |
| | CCAAGATGAGCGAGTGCGTGCTGGGCCAGAGCAAGAGGGTGGACTTCTGC | |
| | GGCAAGGGCTACCACCTGATGAGCTTCCCCCAGAGCGCCCCCCACGGCGT | |
| | GGTGTTCCTGCACGTGACCTACGTGCCCGCCCAGGAGAAGAACTTCACCAC | |
| | CGCCCCCGCCATCTGCCACGACGGCAAGGCCCACTTCCCCAGGGAGGGC | |
| | GTGTTCGTGAGCAACGGCACCCACTGGTTCGTGACCCAGAGGAACTTCTAC | |
| | GAGCCCCAGATCATCACCACCGACAACACCTTCGTGAGCGGCAACTGCGA | |
| | CGTGGTGATCGGCATCGTGAACAACACCGTGTACGACCCCCTGCAGCCCG | |
| | AGCTGGACAGCTTCAAGGAGGAGCTGGACAAGTACTTCAAGAACCACACCA | |
| | GCCCCGACGTGGACCTGGGCGACATCAGCGGCATCAACGCCAGCGTGGTG | |
| | AACATCCAGAAGGAGATCGACAGGCTGAACGAGGTGGCCAAGAACCTGAA | |
| | CGAGAGCCTGATCGACCTGCAGGAGCTGGGCAAGTACGAGCAGTACATCA | |
| | AGTGGCCCTGGTACATCTGGCTGGGCTTCATCGCCGGCCTGATCGCCATC | |
| | GTGATGGTGACCATCATGCTGTGCTGCATGACCAGCTGCTGCAGCTGCCTG | |
| | AAGGGCTGCTGCAGCTGCGGCAGCTGCTGCAAGTTCGACGAGGACGACAG | |
| | CGAGCCCGTGCTGAAGGGCGTGAAGCTGCACTACACCGGAAGCGGAGCCA | |
| | CGAACTTCTCTCTGTTAAAGCAAGCAGGAGATGTTGAAGAAAACCCCGGGC | |
| | CTATGAAGGCAATACTAGTAGTTCTGCTATATACATTTGCAACCGCAAATGC | |
| | AGACACATTATGTATAGGTTATCATGCGAACAACTCAACAGACACTGTAGAC | |
| | ACAGTACTAGAAAAGAATGTAACAGTAACACACTCTGTTAACCTTCTAGAAG | |
| | ACAAGCATAACGGGAAACTATGCAAACTAAGAGGGGTAGCCCCATTGCATT | |
| | TGGGTAAATGTAACATTGCTGGCTGGATCCTGGGAAATCCAGAGTGTGAAT | |
| | CACTCTCCACAGCAAGCTCATGGTCCTACATTGTGGAAACATCTAGTTCAGA | |
| | CAATGGAACGTGTTACCCAGGAGATTTCATCGATTATGAGGAGCTAAGAGA | |
| | GCAATTGAGCTCAGTGTCATCATTTGAAAGGTTTGAGATATTCCCCAAGACA | |
| | AGTTCATGGCCCAATCATGACTCGAACAAAGGTGTAACGACAGCATGTCCT | |
| | CATGCTGGAGCAAAAAGCTTCTACAAAAATTTAATATGGCTAGTTAAAAAAG | |
| | GAAATTCATACCCAAAGCTCAGCAAATCCTACATTAATGATAAAGGGAAAGA | |
| | AGTCCTCGTGCTATGGGGCATTCACCATCCACCTACTAGTGCTGACCAACA | |
| | AAGTCTCTATCAGAATGCAGATGCATATGTTTTTGTGGGGACATCAAGATAC | |
| | AGCAAGAAGTTCAAGCCGGAAATAGCAATAAGGCCCAAAGTGAGGGATCAA | |
| | GAAGGGAGAATGAACTATTACTGGACACTAGTAGAGCCGGGAGACAAAATA | |
| | ACATTCGAAGCAACTGGAAATCTAGTGGTACCGAGATATGCATTCGCAATG | |
| | GAAAGAAATGCTGGATCTGGTATTATCATTTCAGATACACCAGTCCACGATT | |
| | GCAATACAACTTGTCAGACACCCAAGGGTGCTATAAACACCAGCCTCCCATT | |
| | TCAGAACATACATCCGATCACAATTGGAAAATGTCCAAAATATGTAAAAAGC | |
| | ACAAAATTGAGACTGGCCACAGGATTGAGGAATGTCCCGTCCATTCAATCTA | |
| | GAGGCCTATTTGGGGCCATTGCCGGTTTCATTGAAGGGGGGTGGACAGGG | |
| | ATGGTAGATGGATGGTACGGTTATCACCATCAAAATGAGCAGGGGTCAGGA | |
| | TATGCAGCCGACCTGAAGAGCACACAGAATGCCATTGACGAGATTACTAAC | |
| | AAAGTAAACTCTGTTATTGAAAAAATGAATACACAGTTCACAGCAGTAGGTA | |
| | AAGAGTTCAACCACCTGGAAAAAAGAATAGAGAATTTAAATAAAAAAGTTGA | |
| | TGATGGTTTCCTGGACATTTGGACTTACAATGCCGAACTGTTGGTTCTATTG | |
| | GAAAATGAAAGAACTTTGGACTACCACGATTCAAAGGTGAAGAACTTATATG | |
| | AAAAGGTAAGAAGCCAGTTAAAAAACAATGCCAAGGAAATTGGAAACGGCT | |
| | GCTTTGAATTTTACCACAAATGTGATAACACGTGCATGGAAAGTGTCAAAAA | |
| | TGGGACTTATGACTACCCAAAATACTCAGAGGAAGCAAAATTAAACAGAGAA | |
| | GAAATAGATGGGGTAAAGCTGGAATCAACAAGGATTTACCAGATTTTGGCG | |
| | ATCTATTCAACCGTCGCCAGTTCATTGGTACTGGTAGTCTCCCTGGGGGCA | |
| | ATCAGTTTCTGGATGTGCTCTAATGGGTCTCTACAGTGTAGAATATGTATTTA | |
| | AGGAAGCGGAGCCACGAACTTCTCTCTGTTAAAGCAAGCAGGAGATGTTGA | |
| | AGAAAACCCCGGGCCTAGCAAAAGCAGGGGATAATTCTATTAACCATGAAG | |
| | ACTATCATTGCTTTGAGCTACATTCTATGTCTGGTTTTCGCTCAAAAACTTCC | |
| | TGGAAATGACAATAGCACTGCAACGCTGTGCCTTGGGCACCATGCAGTACC | |
| | AAACGGAACGATAGTGAAAACAATCACGAATGACCAAATTGAAGTTACTAAT | |
| | GCTACTGAGCTGGTTCAGAATTCCTCAATAGGTGAAATATGCGACAGTCCTC | |
| | ATCAGATCCTTGATGGAGAAAACTGCACACTAATAGATGCTCTATTGGGAGA | |
| | CCCTCAGTGTGATGGCTTTCAAAATAAGAAATGGGACCTTTTTGTTGAACGA | |
| | AGCAAAGCCTACAGCAACTGTTACCCTTATGATGTGCCGGATTATGCCTCCC | |
| | TTAGGTCACTAGTTGCCTCATCCGGCACACTGGAGTTTAACAATGAAAGCTT | |
| | CAATTGGGCTGGAGTCACTCAAAACGGAACAAGTTCTGCTTGCATAAGGGG | |
| | ATCTAATAGTAGTTTCTTTAGTAGATTAAATTGGTTGACCCACTTAAACTTCA | |
| | AGTACCCAGCATTGAACGTGACTATGCCAAACAATGAACAATTTGACAAATT | |
| | GTACATTTGGGGGGTTCACCACCCGGGTACGGACAAGGACCAAATCTTCCT | |
| | GTATGCTCAATCATCAGGAAGAATCACAGTATCTACCAAAAGAAGCCAACAA | |
| | GCTGTAATCCCGAATATCGGATCTAGACCCAGAATAAGGAATATCCCTAGCA | |
| | GAATAAGCATCTATTGGACAATAGTAAAACCGGGAGACATACTTTTGATTAA | |
| | CAGCACAGGGAATCTAATTGCTCCTAGGGGTTACTTCAAAATACGAAGTGG | |
| | GAAAAGCTCAATAATGAGATCAGATGCACCCATTGGCAAATGCAAGTCTGAA | |
| | TGCATCACTCCAAATGGAAGCATTCCCAATGACAAACCATTCCAAAATGTAA | |
| | ACAGGATCACATACGGGGCCTGTCCCAGATATGTTAAGCAAAGCACTCTGA | |
| | AATTGGCAACAGGAATGCGAAATGTACCAGAGAAACAAACTAGAGGCATATT | |
| | TGGCGCAATAGCGGGTTTCATAGAAAATGGTTGGGAGGGAATGGTGGATG | |
| | GTTGGTACGGTTTCAGGCATCAAAATTCTGAGGGAAGAGGACAAGCAGCAG | |
| | ATCTCAAAAGCACTCAAGCAGCAATCGATCAAATCAATGGGAAGCTGAATCG | |
| | ATTGATCGGGAAAACCAACGAGAAATTCCATCAGATTGAGAAAGAATTCTCA | |
| | GAAGTAGAAGGGAGAATTCAGGACCTTGAGAAATATGTTGAGGACACAAAA | |
| | ATAGATCTCTGGTCATACAACGCAGAGCTTCTTGTTGCCCTGGAAAACCAAC | |
| | ATACAATTGATCTAACTGACTCAGAAATGAACAAACTGTTTGAAAAAACAAAG | |
| | AAGCAACTGAGGGAAAATGCTGAGGATATGGGCAATGGTTGTTTCAAAATAT | |
| | ACCACAAATGTGACAATGCCTGCATAGGATCAATCAGAAATGGAACTTATGA | |
| | CCACGATGTATACAGGGATGAAGCATTAAACAACCGGTTCCAGATCAAGGG | |
| | AGTTGAGCTGAAGTCAGGGTACAAAGATTGGATCCTATGGATTTCCTTTGCC | |
| | ATATCATGTTTTTTGCTTTGTGTTGCTTTGTTGGGGTTCATCATGTGGGCCTG | |
| | CCAAAAGGGCAACATTAGGTGCAACATTTGCATTTGAGTGCATTAATTAAAA | |
| | ACACCCTTGTTTCTACTGGAAGCGGAGCCACGAACTTCTCTCTGTTAAAGCA | |
| | AGCAGGAGATGTTGAAGAAAACCCCGGGCCTATTTTCTAATATCCACAAAAT | |
| | GAAGGCAATAATTGTACTACTCATGGTAGTAACATCCAATGCAGATCGAATC | |
| | TGCACTGGGATAACATCGTCAAACTCACCACATGTCGTCAAAACTGCTACTC | |
| | AAGGGGAGGTCAACGTGACCGGTGTAATACCACTGACAACAACACCCACCA | |
| | AATCTCATTTTGCAAATCTCAAAGGAACAGAAACCAGGGGGAAACTATGCCC | |
| | AAAATGCCTCAACTGCACAGATCTGGATGTAGCCTTGGGCAGACCAAAATG | |
| | CACAGGGAAAATACCCTCTGCAAGGGTTTCAATACTCCATGAAGTCAGACCT | |
| | GTTACATCTGGGTGCTTTCCTATAATGCACGATAGAACAAAAATTAGACAGC | |
| | TGCCTAACCTTCTCCGAGGATACGAACATGTCAGGTTATCAACTCACAACGT | |
| | TATCAATGCAAAAGATGCACCAGGAAGACCCTACGAAATTGGAACCTCAGG | |
| | GTCTTGCCCTAACATTACCAATGGAAACGGATTCTTCGCAACAATGGCTTGG | |
| | GCCGTCCCAAAAAACAAAACAGCAACAAATCCATTAACAATAGAAGTACCAT | |
| | ACATTTGTACAGAAGGAGAAGACCAAATTACCGTTTGGGGGTTCCACTCTGA | |
| | CAACGAGACCCAAATGGCAAAGCTCTATGGGGACTCAAAGCCCCAGAAGTT | |
| | CACCTCATCTGCCAACGGAGTGACCACACATTACGTTTCACAGATTGGTGG | |
| | CTTCCCAAATCAAACAGAAGACGGAGGACTACCACAAAGTGGCAGAATTGT | |
| | TGTTGATTACATGGTGCAGAAATCTGGAAAAACAGGAACAATTACCTATCAA | |
| | AGAGGTATTTTATTGCCTCAAAAAGTGTGGTGCGCAAGTGGCAGGAGCAAG | |
| | GTAATAAAAGGATCCTTGCCCTTAATTGGAGAAGCAGATTGCCTCCATGAAA | |
| | AATACGGTGGATTAAACAAAAGCAAGCCTTACTACACAGGGGAACATGCAA | |
| | AGGCCATAGGAAATTGCCCAATATGGGTGAAAACACCCTTGAAGCTGGCCA | |
| | ATGGAACCAAATATAGACCCCCTGCAAAACTATTAAAGGAAAGAGGTTTCTT | |
| | CGGAGCCATTGCTGGTTTCTTAGAGGGAGGATGGGAAGGAATGATTGCAG | |
| | GTTGGCACGGATACACATCCCATGGGGCACATGGAGTAGCGGTGGCAGCT | |
| | GACCTTAAGAGCACTCAAGAGGCCATAAACAAGATAACAAAAAATCTCAACT | |
| | CTTTGAGTGAGCTGGAAGTAAAGAATCTTCAAAGACTAAGCGGTGCCATGG | |
| | ATGAACTCCACAACGAAATACTAGAACTAGATGAGAAAGTGGATGATCTCAG | |
| | AGCTGACACAATAAGCTCACAAATAGAACTCGCAGTCCTGCTTTCCAATGAA | |
| | GGAATAATAAACAGTGAAGATGAACATCTCTTGGCGCTTGAAAGAAAGCTGA | |
| | AGAAAATGCTGGGCCCCTCTGCTGTAGAGATAGGGAATGGATGCTTTGAAA | |
| | CCAAACACAAGTGCAACCAGACCTGCCTCGACAGAATAGCTGCTGGTACCT | |
| | TTGATGCAGGAGAATTTTCTCTCCCCACCTTTGATTCACTGAATATTACTGCT | |
| | GCATCTTTAAATGACGACGGATTGGACAATCATACTATACTGCTTTACTACTC | |
| | AACTGCTGCCTCCAGTTTGGCTGTAACACTGATGATAGCTATCTTTGTTGTT | |
| | TATATGGTCTCCAGAGACAATGTTTCTTGCTCCATTTGTCTATAAGGAAAGTT | |
| | AAGCCCTGTATTTTCCTTTATTGTAGTGCTTGTTTGCTTGTTGTCATTACAAA | |
| | GAAACGTTATTGAAAAATGGAAGCGGAGCCACGAACTTCTCTCTGTTAAAGC | |
| | AAGCAGGAGATGTTGAAGAAAACCCCGGGCCTTTCGGAGCTATTGCTGGTT | |
| | TCTTGGAAGGAGGATGGGAAGGAATGATTGCAGGTTGGCACGGATACACAT | |
| | CTCATGGAGCACATGGAGTAGCAGTGGCAGCAGACCTTAAGAGTACCCATG | |
| | ATGACTCGAGCTGGTACTGCATGCACGCAATGCTAGCTGCCCCTTTCCCGT | |
| | CCTGGGTACCCCGAGTCTCCCCCGACCTCGGGTCCCAGGTATGCTCCCAC | |
| | CTCCACCTGCCCCACTCACCACCTCTGCTAGTTCCAGACACCTCCCAAGCA | |
| | CGCAGCAATGCAGCTCAAAACGCTTAGCCTAGCCACACCCCCACGGGAAAC | |
| | AGCAGTGATTAACCTTTAGCAATAAACGAAAGTTTAACTAAGCTATACTAACC | |
| | CCAGGGTTGGTCAATTTCGTGCCAGCCACACCCTGGAGCTAGCAAAAAAAA | |
| Pan-CoV- | TAGTTATTAATAGTAATCAATTACGGGGTCATTAGTTCATAGCCCATATATGG | 35 |
| Flu | AGTTCCGCGTTACATAACTTACGGTAAATGGCCCGCCTGGCTGACCGCCCA | |
| Vaccine | ACGACCCCCGCCCATTGACGTCAATAATGACGTATGTTCCCATAGTAACGC | |
| #15 CoV- | CAATAGGGACTTTCCATTGACGTCAATGGGTGGAGTATTTACGGTAAACTGC | |
| Omicron | CCACTTGGCAGTACATCAAGTGTATCATATGCCAAGTACGCCCCCTATTGAC | |
| BA.2 Spike | GTCAATGACGGTAAATGGCCCGCCTGGCATTATGCCCAGTACATGACCTTA | |
| (HexaPro)- | TGGGACTTTCCTACTTGGCAGTACATCTACGTATTAGTCATCGCTATTACCA | |
| Influenza | TGGTGATGCGGTTTTGGCAGTACATCAATGGGCGTGGATAGCGGTTTGACT | |
| (HA) | CACGGGGATTTCCAAGTCTCCACCCCATTGACGTCAATGGGAGTTTGTTTTG | |
| | GCACCAAAATCAACGGGACTTTCCAAAATGTCGTAACAACTCCGCCCCATTG | |
| | ACGCAAATGGGGGGTAGGCGTGTACGGTGGGAGGTCTATATAAGCAGAGC | |
| | TGGTTTAGTGAACCGTCAGATCGAGAATAAACTAGTATTCTTCTGGTCCCCA | |
| | CAGACTCAGAGAGAACCCGCCACCATGTTCGTGTTCCTGGTGCTGCTGCCC | |
| | CTGGTGAGCAGCCAGTGCGTGAACCTGATCACCAGGACCCAGAGCTACAC | |
| | CAACAGCTTCACCAGGGGCGTGTACTACCCCGACAAGGTGTTCAGGAGCA | |
| | GCGTGCTGCACAGCACCCAGGACCTGTTCCTGCCCTTCTTCAGCAACGTGA | |
| | CCTGGTTCCACGCCATCCACGTGAGCGGCACCAACGGCACCAAGAGGTTC | |
| | GACAACCCCGTGCTGCCCTTCAACGACGGCGTGTACTTCGCCAGCACCGA | |
| | GAAGAGCAACATCATCAGGGGCTGGATCTTCGGCACCACCCTGGACAGCA | |
| | AGACCCAGAGCCTGCTGATCGTGAACAACGCCACCAACGTGGTGATCAAG | |
| | GTGTGCGAGTTCCAGTTCTGCAACGACCCCTTCCTGGACGTGTACTACCAC | |
| | AAGAACAACAAGAGCTGGATGGAGAGCGAGTTCAGGGTGTACAGCAGCGC | |
| | CAACAACTGCACCTTCGAGTACGTGAGCCAGCCCTTCCTGATGGACCTGGA | |
| | GGGCAAGCAGGGCAACTTCAAGAACCTGAGGGAGTTCGTGTTCAAGAACAT | |
| | CGACGGCTACTTCAAGATCTACAGCAAGCACACCCCCATCAACCTGGGCAG | |
| | GGACCTGCCCCAGGGCTTCAGCGCCCTGGAGCCCCTGGTGGACCTGCCCA | |
| | TCGGCATCAACATCACCAGGTTCCAGACCCTGCTGGCCCTGCACAGGAGCT | |
| | ACCTGACCCCCGGCGACAGCAGCAGCGGCTGGACCGCCGGCGCCGCCGC | |
| | CTACTACGTGGGCTACCTGCAGCCCAGGACCTTCCTGCTGAAGTACAACGA | |
| | GAACGGCACCATCACCGACGCCGTGGACTGCGCCCTGGACCCCCTGAGCG | |
| | AGACCAAGTGCACCCTGAAGAGCTTCACCGTGGAGAAGGGCATCTACCAGA | |
| | CCAGCAACTTCAGGGTGCAGCCCACCGAGAGCATCGTGAGGTTCCCCAAC | |
| | ATCACCAACCTGTGCCCCTTCGACGAGGTGTTCAACGCCACCAGGTTCGCC | |
| | AGCGTGTACGCCTGGAACAGGAAGAGGATCAGCAACTGCGTGGCCGACTA | |
| | CAGCGTGCTGTACAACTTCGCCCCCTTCTTCGCCTTCAAGTGCTACGGCGT | |
| | GAGCCCCACCAAGCTGAACGACCTGTGCTTCACCAACGTGTACGCCGACA | |
| | GCTTCGTGATCAGGGGCAACGAGGTGAGCCAGATCGCCCCCGGCCAGACC | |
| | GGCAACATCGCCGACTACAACTACAAGCTGCCCGACGACTTCACCGGCTGC | |
| | GTGATCGCCTGGAACAGCAACAAGCTGGACAGCAAGGTGGGCGGCAACTA | |
| | CAACTACCTGTACAGGCTGTTCAGGAAGAGCAACCTGAAGCCCTTCGAGAG | |
| | GGACATCAGCACCGAGATCTACCAGGCCGGCAACAAGCCCTGCAACGGCG | |
| | TGGCCGGCTTCAACTGCTACTTCCCCCTGAGGAGCTACGGCTTCAGGCCCA | |
| | CCTACGGCGTGGGCCACCAGCCCTACAGGGTGGTGGTGCTGAGCTTCGAG | |
| | CTGCTGCACGCCCCCGCCACCGTGTGCGGCCCCAAGAAGAGCACCAACCT | |
| | GGTGAAGAACAAGTGCGTGAACTTCAACTTCAACGGCCTGACCGGCACCG | |
| | GCGTGCTGACCGAGAGCAACAAGAAGTTCCTGCCCTTCCAGCAGTTCGGCA | |
| | GGGACATCGCCGACACCACCGACGCCGTGAGGGACCCCCAGACCCTGGA | |
| | GATCCTGGACATCACCCCCTGCAGCTTCGGCGGCGTGAGCGTGATCACCC | |
| | CCGGCACCAACACCAGCAACCAGGTGGCCGTGCTGTACCAGGGCGTGAAC | |
| | TGCACCGAGGTGCCCGTGGCCATCCACGCCGACCAGCTGACCCCCACCTG | |
| | GAGGGTGTACAGCACCGGCAGCAACGTGTTCCAGACCAGGGCCGGCTGCC | |
| | TGATCGGCGCCGAGTACGTGAACAACAGCTACGAGTGCGACATCCCCATC | |
| | GGCGCCGGCATCTGCGCCAGCTACCAGACCCAGACCAAGAGCCACCAGCA | |
| | GGCCCAGAGCGTGGCCAGCCAGAGCATCATCGCCTACACCATGAGCCTGG | |
| | GCGCCGAGAACAGCGTGGCCTACAGCAACAACAGCATCGCCATCCCCACC | |
| | AACTTCACCATCAGCGTGACCACCGAGATCCTGCCCGTGAGCATGACCAAG | |
| | ACCAGCGTGGACTGCACCATGTACATCTGCGGCGACAGCACCGAGTGCAG | |
| | CAACCTGCTGCTGCAGTACGGCAGCTTCTGCACCCAGCTGAAGAGGGCCC | |
| | TGACCGGCATCGCCGTGGAGCAGGACAAGAACACCCAGGAGGTGTTCGCC | |
| | CAGGTGAAGCAGATCTACAAGACCCCCCCCATCAAGTACTTCGGCGGCTTC | |
| | AACTTCAGCCAGATCCTGCCCGACCCCAGCAAGCCCAGCAAGAGGAGCCC | |
| | CATCGAGGACCTGCTGTTCAACAAGGTGACCCTGGCCGACGCCGGCTTCAT | |
| | CAAGCAGTACGGCGACTGCCTGGGCGACATCGCCGCCAGGGACCTGATCT | |
| | GCGCCCAGAAGTTCAACGGCCTGACCGTGCTGCCCCCCCTGCTGACCGAC | |
| | GAGATGATCGCCCAGTACACCAGCGCCCTGCTGGCCGGCACCATCACCAG | |
| | CGGCTGGACCTTCGGCGCCGGCCCCGCCCTGCAGATCCCCTTCCCCATGC | |
| | AGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATC | |
| | CAGGACAGCCTGAGCAGCACCCCCAGCGCCCTGGGCAAGCTGCAGGACGT | |
| | GGTGAACCACAACGCCCAGGCCCTGAACACCCTGGTGAAGCAGCTGAGCA | |
| | GCAAGTTCGGCGCCATCAGCAGCGTGCTGAACGACATCCTGAGCAGGCTG | |
| | GACCCCCCCGAGGCCGAGGTGCAGATCGACAGGCTGATCACCGGCAGGCT | |
| | GCAGAGCCTGCAGACCTACGTGACCCAGCAGCTGATCAGGGCCGCCGAGA | |
| | TCAGGGCCAGCGCCAACCTGGCCGCCACCAAGATGAGCGAGTGCGTGCTG | |
| | GGCCAGAGCAAGAGGGTGGACTTCTGCGGCAAGGGCTACCACCTGATGAG | |
| | CTTCCCCCAGAGCGCCCCCCACGGCGTGGTGTTCCTGCACGTGACCTACG | |
| | TGCCCGCCCAGGAGAAGAACTTCACCACCGCCCCCGCCATCTGCCACGAC | |
| | GGCAAGGCCCACTTCCCCAGGGAGGGCGTGTTCGTGAGCAACGGCACCCA | |
| | CTGGTTCGTGACCCAGAGGAACTTCTACGAGCCCCAGATCATCACCACCGA | |
| | CAACACCTTCGTGAGCGGCAACTGCGACGTGGTGATCGGCATCGTGAACAA | |
| | CACCGTGTACGACCCCCTGCAGCCCGAGCTGGACAGCTTCAAGGAGGAGC | |
| | TGGACAAGTACTTCAAGAACCACACCAGCCCCGACGTGGACCTGGGCGAC | |
| | ATCAGCGGCATCAACGCCAGCGTGGTGAACATCCAGAAGGAGATCGACAG | |
| | GCTGAACGAGGTGGCCAAGAACCTGAACGAGAGCCTGATCGACCTGCAGG | |
| | AGCTGGGCAAGTACGAGCAGTACATCAAGTGGCCCTGGTACATCTGGCTG | |
| | GGCTTCATCGCCGGCCTGATCGCCATCGTGATGGTGACCATCATGCTGTGC | |
| | TGCATGACCAGCTGCTGCAGCTGCCTGAAGGGCTGCTGCAGCTGCGGCAG | |
| | CTGCTGCAAGTTCGACGAGGACGACAGCGAGCCCGTGCTGAAGGGCGTGA | |
| | AGCTGCACTACACCGGAAGCGGAGCCACGAACTTCTCTCTGTTAAAGCAAG | |
| | CAGGAGATGTTGAAGAAAACCCCGGGCCTATGAAGGCAATACTAGTAGTTC | |
| | TGCTATATACATTTGCAACCGCAAATGCAGACACATTATGTATAGGTTATCAT | |
| | GCGAACAACTCAACAGACACTGTAGACACAGTACTAGAAAAGAATGTAACA | |
| | GTAACACACTCTGTTAACCTTCTAGAAGACAAGCATAACGGGAAACTATGCA | |
| | AACTAAGAGGGGTAGCCCCATTGCATTTGGGTAAATGTAACATTGCTGGCT | |
| | GGATCCTGGGAAATCCAGAGTGTGAATCACTCTCCACAGCAAGCTCATGGT | |
| | CCTACATTGTGGAAACATCTAGTTCAGACAATGGAACGTGTTACCCAGGAGA | |
| | TTTCATCGATTATGAGGAGCTAAGAGAGCAATTGAGCTCAGTGTCATCATTT | |
| | GAAAGGTTTGAGATATTCCCCAAGACAAGTTCATGGCCCAATCATGACTCGA | |
| | ACAAAGGTGTAACGACAGCATGTCCTCATGCTGGAGCAAAAAGCTTCTACA | |
| | AAAATTTAATATGGCTAGTTAAAAAAGGAAATTCATACCCAAAGCTCAGCAAA | |
| | TCCTACATTAATGATAAAGGGAAAGAAGTCCTCGTGCTATGGGGCATTCACC | |
| | ATCCACCTACTAGTGCTGACCAACAAAGTCTCTATCAGAATGCAGATGCATA | |
| | TGTTTTTGTGGGGACATCAAGATACAGCAAGAAGTTCAAGCCGGAAATAGC | |
| | AATAAGGCCCAAAGTGAGGGATCAAGAAGGGAGAATGAACTATTACTGGAC | |
| | ACTAGTAGAGCCGGGAGACAAAATAACATTCGAAGCAACTGGAAATCTAGT | |
| | GGTACCGAGATATGCATTCGCAATGGAAAGAAATGCTGGATCTGGTATTATC | |
| | ATTTCAGATACACCAGTCCACGATTGCAATACAACTTGTCAGACACCCAAGG | |
| | GTGCTATAAACACCAGCCTCCCATTTCAGAACATACATCCGATCACAATTGG | |
| | AAAATGTCCAAAATATGTAAAAAGCACAAAATTGAGACTGGCCACAGGATTG | |
| | AGGAATGTCCCGTCCATTCAATCTAGAGGCCTATTTGGGGCCATTGCCGGT | |
| | TTCATTGAAGGGGGGTGGACAGGGATGGTAGATGGATGGTACGGTTATCAC | |
| | CATCAAAATGAGCAGGGGTCAGGATATGCAGCCGACCTGAAGAGCACACA | |
| | GAATGCCATTGACGAGATTACTAACAAAGTAAACTCTGTTATTGAAAAAATGA | |
| | ATACACAGTTCACAGCAGTAGGTAAAGAGTTCAACCACCTGGAAAAAAGAAT | |
| | AGAGAATTTAAATAAAAAAGTTGATGATGGTTTCCTGGACATTTGGACTTACA | |
| | ATGCCGAACTGTTGGTTCTATTGGAAAATGAAAGAACTTTGGACTACCACGA | |
| | TTCAAAGGTGAAGAACTTATATGAAAAGGTAAGAAGCCAGTTAAAAAACAAT | |
| | GCCAAGGAAATTGGAAACGGCTGCTTTGAATTTTACCACAAATGTGATAACA | |
| | CGTGCATGGAAAGTGTCAAAAATGGGACTTATGACTACCCAAAATACTCAGA | |
| | GGAAGCAAAATTAAACAGAGAAGAAATAGATGGGGTAAAGCTGGAATCAAC | |
| | AAGGATTTACCAGATTTTGGCGATCTATTCAACCGTCGCCAGTTCATTGGTA | |
| | CTGGTAGTCTCCCTGGGGGCAATCAGTTTCTGGATGTGCTCTAATGGGTCT | |
| | CTACAGTGTAGAATATGTATTTAAGGAAGCGGAGCCACGAACTTCTCTCTGT | |
| | TAAAGCAAGCAGGAGATGTTGAAGAAAACCCCGGGCCTAGCAAAAGCAGG | |
| | GGATAATTCTATTAACCATGAAGACTATCATTGCTTTGAGCTACATTCTATGT | |
| | CTGGTTTTCGCTCAAAAACTTCCTGGAAATGACAATAGCACTGCAACGCTGT | |
| | GCCTTGGGCACCATGCAGTACCAAACGGAACGATAGTGAAAACAATCACGA | |
| | ATGACCAAATTGAAGTTACTAATGCTACTGAGCTGGTTCAGAATTCCTCAAT | |
| | AGGTGAAATATGCGACAGTCCTCATCAGATCCTTGATGGAGAAAACTGCAC | |
| | ACTAATAGATGCTCTATTGGGAGACCCTCAGTGTGATGGCTTTCAAAATAAG | |
| | AAATGGGACCTTTTTGTTGAACGAAGCAAAGCCTACAGCAACTGTTACCCTT | |
| | ATGATGTGCCGGATTATGCCTCCCTTAGGTCACTAGTTGCCTCATCCGGCA | |
| | CACTGGAGTTTAACAATGAAAGCTTCAATTGGGCTGGAGTCACTCAAAACG | |
| | GAACAAGTTCTGCTTGCATAAGGGGATCTAATAGTAGTTTCTTTAGTAGATTA | |
| | AATTGGTTGACCCACTTAAACTTCAAGTACCCAGCATTGAACGTGACTATGC | |
| | CAAACAATGAACAATTTGACAAATTGTACATTTGGGGGGTTCACCACCCGGG | |
| | TACGGACAAGGACCAAATCTTCCTGTATGCTCAATCATCAGGAAGAATCACA | |
| | GTATCTACCAAAAGAAGCCAACAAGCTGTAATCCCGAATATCGGATCTAGAC | |
| | CCAGAATAAGGAATATCCCTAGCAGAATAAGCATCTATTGGACAATAGTAAA | |
| | ACCGGGAGACATACTTTTGATTAACAGCACAGGGAATCTAATTGCTCCTAGG | |
| | GGTTACTTCAAAATACGAAGTGGGAAAAGCTCAATAATGAGATCAGATGCAC | |
| | CCATTGGCAAATGCAAGTCTGAATGCATCACTCCAAATGGAAGCATTCCCAA | |
| | TGACAAACCATTCCAAAATGTAAACAGGATCACATACGGGGCCTGTCCCAG | |
| | ATATGTTAAGCAAAGCACTCTGAAATTGGCAACAGGAATGCGAAATGTACCA | |
| | GAGAAACAAACTAGAGGCATATTTGGCGCAATAGCGGGTTTCATAGAAAAT | |
| | GGTTGGGAGGGAATGGTGGATGGTTGGTACGGTTTCAGGCATCAAAATTCT | |
| | GAGGGAAGAGGACAAGCAGCAGATCTCAAAAGCACTCAAGCAGCAATCGAT | |
| | CAAATCAATGGGAAGCTGAATCGATTGATCGGGAAAACCAACGAGAAATTC | |
| | CATCAGATTGAGAAAGAATTCTCAGAAGTAGAAGGGAGAATTCAGGACCTT | |
| | GAGAAATATGTTGAGGACACAAAAATAGATCTCTGGTCATACAACGCAGAGC | |
| | TTCTTGTTGCCCTGGAAAACCAACATACAATTGATCTAACTGACTCAGAAAT | |
| | GAACAAACTGTTTGAAAAAACAAAGAAGCAACTGAGGGAAAATGCTGAGGA | |
| | TATGGGCAATGGTTGTTTCAAAATATACCACAAATGTGACAATGCCTGCATA | |
| | GGATCAATCAGAAATGGAACTTATGACCACGATGTATACAGGGATGAAGCAT | |
| | TAAACAACCGGTTCCAGATCAAGGGAGTTGAGCTGAAGTCAGGGTACAAAG | |
| | ATTGGATCCTATGGATTTCCTTTGCCATATCATGTTTTTTGCTTTGTGTTGCT | |
| | TTGTTGGGGTTCATCATGTGGGCCTGCCAAAAGGGCAACATTAGGTGCAAC | |
| | ATTTGCATTTGAGTGCATTAATTAAAAACACCCTTGTTTCTACTGGAAGCGGA | |
| | GCCACGAACTTCTCTCTGTTAAAGCAAGCAGGAGATGTTGAAGAAAACCCC | |
| | GGGCCTATTTTCTAATATCCACAAAATGAAGGCAATAATTGTACTACTCATG | |
| | GTAGTAACATCCAATGCAGATCGAATCTGCACTGGGATAACATCGTCAAACT | |
| | CACCACATGTCGTCAAAACTGCTACTCAAGGGGAGGTCAACGTGACCGGTG | |
| | TAATACCACTGACAACAACACCCACCAAATCTCATTTTGCAAATCTCAAAGG | |
| | AACAGAAACCAGGGGGAAACTATGCCCAAAATGCCTCAACTGCACAGATCT | |
| | GGATGTAGCCTTGGGCAGACCAAAATGCACAGGGAAAATACCCTCTGCAAG | |
| | GGTTTCAATACTCCATGAAGTCAGACCTGTTACATCTGGGTGCTTTCCTATA | |
| | ATGCACGATAGAACAAAAATTAGACAGCTGCCTAACCTTCTCCGAGGATACG | |
| | AACATGTCAGGTTATCAACTCACAACGTTATCAATGCAAAAGATGCACCAGG | |
| | AAGACCCTACGAAATTGGAACCTCAGGGTCTTGCCCTAACATTACCAATGGA | |
| | AACGGATTCTTCGCAACAATGGCTTGGGCCGTCCCAAAAAACAAAACAGCA | |
| | ACAAATCCATTAACAATAGAAGTACCATACATTTGTACAGAAGGAGAAGACC | |
| | AAATTACCGTTTGGGGGTTCCACTCTGACAACGAGACCCAAATGGCAAAGC | |
| | TCTATGGGGACTCAAAGCCCCAGAAGTTCACCTCATCTGCCAACGGAGTGA | |
| | CCACACATTACGTTTCACAGATTGGTGGCTTCCCAAATCAAACAGAAGACGG | |
| | AGGACTACCACAAAGTGGCAGAATTGTTGTTGATTACATGGTGCAGAAATCT | |
| | GGAAAAACAGGAACAATTACCTATCAAAGAGGTATTTTATTGCCTCAAAAAG | |
| | TGTGGTGCGCAAGTGGCAGGAGCAAGGTAATAAAAGGATCCTTGCCCTTAA | |
| | TTGGAGAAGCAGATTGCCTCCATGAAAAATACGGTGGATTAAACAAAAGCAA | |
| | GCCTTACTACACAGGGGAACATGCAAAGGCCATAGGAAATTGCCCAATATG | |
| | GGTGAAAACACCCTTGAAGCTGGCCAATGGAACCAAATATAGACCCCCTGC | |
| | AAAACTATTAAAGGAAAGAGGTTTCTTCGGAGCCATTGCTGGTTTCTTAGAG | |
| | GGAGGATGGGAAGGAATGATTGCAGGTTGGCACGGATACACATCCCATGG | |
| | GGCACATGGAGTAGCGGTGGCAGCTGACCTTAAGAGCACTCAAGAGGCCA | |
| | TAAACAAGATAACAAAAAATCTCAACTCTTTGAGTGAGCTGGAAGTAAAGAA | |
| | TCTTCAAAGACTAAGCGGTGCCATGGATGAACTCCACAACGAAATACTAGAA | |
| | CTAGATGAGAAAGTGGATGATCTCAGAGCTGACACAATAAGCTCACAAATAG | |
| | AACTCGCAGTCCTGCTTTCCAATGAAGGAATAATAAACAGTGAAGATGAACA | |
| | TCTCTTGGCGCTTGAAAGAAAGCTGAAGAAAATGCTGGGCCCCTCTGCTGT | |
| | AGAGATAGGGAATGGATGCTTTGAAACCAAACACAAGTGCAACCAGACCTG | |
| | CCTCGACAGAATAGCTGCTGGTACCTTTGATGCAGGAGAATTTTCTCTCCCC | |
| | ACCTTTGATTCACTGAATATTACTGCTGCATCTTTAAATGACGACGGATTGG | |
| | ACAATCATACTATACTGCTTTACTACTCAACTGCTGCCTCCAGTTTGGCTGTA | |
| | ACACTGATGATAGCTATCTTTGTTGTTTATATGGTCTCCAGAGACAATGTTTC | |
| | TTGCTCCATTTGTCTATAAGGAAAGTTAAGCCCTGTATTTTCCTTTATTGTAG | |
| | TGCTTGTTTGCTTGTTGTCATTACAAAGAAACGTTATTGAAAAATTGATGACT | |
| | CGAGCTGGTACTGCATGCACGCAATGCTAGCTGCCCCTTTCCCGTCCTGGG | |
| | TACCCCGAGTCTCCCCCGACCTCGGGTCCCAGGTATGCTCCCACCTCCACC | |
| | TGCCCCACTCACCACCTCTGCTAGTTCCAGACACCTCCCAAGCACGCAGCA | |
| | ATGCAGCTCAAAACGCTTAGCCTAGCCACACCCCCACGGGAAACAGCAGTG | |
| | ATTAACCTTTAGCAATAAACGAAAGTTTAACTAAGCTATACTAACCCCAGGGT | |
| | TGGTCAATTTCGTGCCAGCCACACCCTGGAGCTAGCAAAAAAAA | |
| Pan-CoV- | TAGTTATTAATAGTAATCAATTACGGGGTCATTAGTTCATAGCCCATATATGG | 36 |
| Flu | AGTTCCGCGTTACATAACTTACGGTAAATGGCCCGCCTGGCTGACCGCCCA | |
| Vaccine | ACGACCCCCGCCCATTGACGTCAATAATGACGTATGTTCCCATAGTAACGC | |
| #16 CoV- | CAATAGGGACTTTCCATTGACGTCAATGGGTGGAGTATTTACGGTAAACTGC | |
| Omicron | CCACTTGGCAGTACATCAAGTGTATCATATGCCAAGTACGCCCCCTATTGAC | |
| BA.2 Spike | GTCAATGACGGTAAATGGCCCGCCTGGCATTATGCCCAGTACATGACCTTA | |
| (2 proline | TGGGACTTTCCTACTTGGCAGTACATCTACGTATTAGTCATCGCTATTACCA | |
| subst.)- | TGGTGATGCGGTTTTGGCAGTACATCAATGGGCGTGGATAGCGGTTTGACT | |
| Influenza | CACGGGGATTTCCAAGTCTCCACCCCATTGACGTCAATGGGAGTTTGTTTTG | |
| (HA) | GCACCAAAATCAACGGGACTTTCCAAAATGTCGTAACAACTCCGCCCCATTG | |
| | ACGCAAATGGGGGGTAGGCGTGTACGGTGGGAGGTCTATATAAGCAGAGC | |
| | TGGTTTAGTGAACCGTCAGATCGAGAATAAACTAGTATTCTTCTGGTCCCCA | |
| | CAGACTCAGAGAGAACCCGCCACCATGTTCGTGTTCCTGGTGCTGCTGCCC | |
| | CTGGTGAGCAGCCAGTGCGTGAACCTGATCACCAGGACCCAGAGCTACAC | |
| | CAACAGCTTCACCAGGGGCGTGTACTACCCCGACAAGGTGTTCAGGAGCA | |
| | GCGTGCTGCACAGCACCCAGGACCTGTTCCTGCCCTTCTTCAGCAACGTGA | |
| | CCTGGTTCCACGCCATCCACGTGAGCGGCACCAACGGCACCAAGAGGTTC | |
| | GACAACCCCGTGCTGCCCTTCAACGACGGCGTGTACTTCGCCAGCACCGA | |
| | GAAGAGCAACATCATCAGGGGCTGGATCTTCGGCACCACCCTGGACAGCA | |
| | AGACCCAGAGCCTGCTGATCGTGAACAACGCCACCAACGTGGTGATCAAG | |
| | GTGTGCGAGTTCCAGTTCTGCAACGACCCCTTCCTGGACGTGTACTACCAC | |
| | AAGAACAACAAGAGCTGGATGGAGAGCGAGTTCAGGGTGTACAGCAGCGC | |
| | CAACAACTGCACCTTCGAGTACGTGAGCCAGCCCTTCCTGATGGACCTGGA | |
| | GGGCAAGCAGGGCAACTTCAAGAACCTGAGGGAGTTCGTGTTCAAGAACAT | |
| | CGACGGCTACTTCAAGATCTACAGCAAGCACACCCCCATCAACCTGGGCAG | |
| | GGACCTGCCCCAGGGCTTCAGCGCCCTGGAGCCCCTGGTGGACCTGCCCA | |
| | TCGGCATCAACATCACCAGGTTCCAGACCCTGCTGGCCCTGCACAGGAGCT | |
| | ACCTGACCCCCGGCGACAGCAGCAGCGGCTGGACCGCCGGCGCCGCCGC | |
| | CTACTACGTGGGCTACCTGCAGCCCAGGACCTTCCTGCTGAAGTACAACGA | |
| | GAACGGCACCATCACCGACGCCGTGGACTGCGCCCTGGACCCCCTGAGCG | |
| | AGACCAAGTGCACCCTGAAGAGCTTCACCGTGGAGAAGGGCATCTACCAGA | |
| | CCAGCAACTTCAGGGTGCAGCCCACCGAGAGCATCGTGAGGTTCCCCAAC | |
| | ATCACCAACCTGTGCCCCTTCGACGAGGTGTTCAACGCCACCAGGTTCGCC | |
| | AGCGTGTACGCCTGGAACAGGAAGAGGATCAGCAACTGCGTGGCCGACTA | |
| | CAGCGTGCTGTACAACTTCGCCCCCTTCTTCGCCTTCAAGTGCTACGGCGT | |
| | GAGCCCCACCAAGCTGAACGACCTGTGCTTCACCAACGTGTACGCCGACA | |
| | GCTTCGTGATCAGGGGCAACGAGGTGAGCCAGATCGCCCCCGGCCAGACC | |
| | GGCAACATCGCCGACTACAACTACAAGCTGCCCGACGACTTCACCGGCTGC | |
| | GTGATCGCCTGGAACAGCAACAAGCTGGACAGCAAGGTGGGCGGCAACTA | |
| | CAACTACCTGTACAGGCTGTTCAGGAAGAGCAACCTGAAGCCCTTCGAGAG | |
| | GGACATCAGCACCGAGATCTACCAGGCCGGCAACAAGCCCTGCAACGGCG | |
| | TGGCCGGCTTCAACTGCTACTTCCCCCTGAGGAGCTACGGCTTCAGGCCCA | |
| | CCTACGGCGTGGGCCACCAGCCCTACAGGGTGGTGGTGCTGAGCTTCGAG | |
| | CTGCTGCACGCCCCCGCCACCGTGTGCGGCCCCAAGAAGAGCACCAACCT | |
| | GGTGAAGAACAAGTGCGTGAACTTCAACTTCAACGGCCTGACCGGCACCG | |
| | GCGTGCTGACCGAGAGCAACAAGAAGTTCCTGCCCTTCCAGCAGTTCGGCA | |
| | GGGACATCGCCGACACCACCGACGCCGTGAGGGACCCCCAGACCCTGGA | |
| | GATCCTGGACATCACCCCCTGCAGCTTCGGCGGCGTGAGCGTGATCACCC | |
| | CCGGCACCAACACCAGCAACCAGGTGGCCGTGCTGTACCAGGGCGTGAAC | |
| | TGCACCGAGGTGCCCGTGGCCATCCACGCCGACCAGCTGACCCCCACCTG | |
| | GAGGGTGTACAGCACCGGCAGCAACGTGTTCCAGACCAGGGCCGGCTGCC | |
| | TGATCGGCGCCGAGTACGTGAACAACAGCTACGAGTGCGACATCCCCATC | |
| | GGCGCCGGCATCTGCGCCAGCTACCAGACCCAGACCAAGAGCCACCAGCA | |
| | GGCCCAGAGCGTGGCCAGCCAGAGCATCATCGCCTACACCATGAGCCTGG | |
| | GCGCCGAGAACAGCGTGGCCTACAGCAACAACAGCATCGCCATCCCCACC | |
| | AACTTCACCATCAGCGTGACCACCGAGATCCTGCCCGTGAGCATGACCAAG | |
| | ACCAGCGTGGACTGCACCATGTACATCTGCGGCGACAGCACCGAGTGCAG | |
| | CAACCTGCTGCTGCAGTACGGCAGCTTCTGCACCCAGCTGAAGAGGGCCC | |
| | TGACCGGCATCGCCGTGGAGCAGGACAAGAACACCCAGGAGGTGTTCGCC | |
| | CAGGTGAAGCAGATCTACAAGACCCCCCCCATCAAGTACTTCGGCGGCTTC | |
| | AACTTCAGCCAGATCCTGCCCGACCCCAGCAAGCCCAGCAAGAGGAGCTT | |
| | CATCGAGGACCTGCTGTTCAACAAGGTGACCCTGGCCGACGCCGGCTTCAT | |
| | CAAGCAGTACGGCGACTGCCTGGGCGACATCGCCGCCAGGGACCTGATCT | |
| | GCGCCCAGAAGTTCAACGGCCTGACCGTGCTGCCCCCCCTGCTGACCGAC | |
| | GAGATGATCGCCCAGTACACCAGCGCCCTGCTGGCCGGCACCATCACCAG | |
| | CGGCTGGACCTTCGGCGCCGGCGCCGCCCTGCAGATCCCCTTCGCCATGC | |
| | AGATGGCCTACAGGTTCAACGGCATCGGCGTGACCCAGAACGTGCTGTAC | |
| | GAGAACCAGAAGCTGATCGCCAACCAGTTCAACAGCGCCATCGGCAAGATC | |
| | CAGGACAGCCTGAGCAGCACCGCCAGCGCCCTGGGCAAGCTGCAGGACG | |
| | TGGTGAACCACAACGCCCAGGCCCTGAACACCCTGGTGAAGCAGCTGAGC | |
| | AGCAAGTTCGGCGCCATCAGCAGCGTGCTGAACGACATCCTGAGCAGGCT | |
| | GGACCCCCCCGAGGCCGAGGTGCAGATCGACAGGCTGATCACCGGCAGG | |
| | CTGCAGAGCCTGCAGACCTACGTGACCCAGCAGCTGATCAGGGCCGCCGA | |
| | GATCAGGGCCAGCGCCAACCTGGCCGCCACCAAGATGAGCGAGTGCGTGC | |
| | TGGGCCAGAGCAAGAGGGTGGACTTCTGCGGCAAGGGCTACCACCTGATG | |
| | AGCTTCCCCCAGAGCGCCCCCCACGGCGTGGTGTTCCTGCACGTGACCTA | |
| | CGTGCCCGCCCAGGAGAAGAACTTCACCACCGCCCCCGCCATCTGCCACG | |
| | ACGGCAAGGCCCACTTCCCCAGGGAGGGCGTGTTCGTGAGCAACGGCACC | |
| | CACTGGTTCGTGACCCAGAGGAACTTCTACGAGCCCCAGATCATCACCACC | |
| | GACAACACCTTCGTGAGCGGCAACTGCGACGTGGTGATCGGCATCGTGAA | |
| | CAACACCGTGTACGACCCCCTGCAGCCCGAGCTGGACAGCTTCAAGGAGG | |
| | AGCTGGACAAGTACTTCAAGAACCACACCAGCCCCGACGTGGACCTGGGC | |
| | GACATCAGCGGCATCAACGCCAGCGTGGTGAACATCCAGAAGGAGATCGA | |
| | CAGGCTGAACGAGGTGGCCAAGAACCTGAACGAGAGCCTGATCGACCTGC | |
| | AGGAGCTGGGCAAGTACGAGCAGTACATCAAGTGGCCCTGGTACATCTGG | |
| | CTGGGCTTCATCGCCGGCCTGATCGCCATCGTGATGGTGACCATCATGCTG | |
| | TGCTGCATGACCAGCTGCTGCAGCTGCCTGAAGGGCTGCTGCAGCTGCGG | |
| | CAGCTGCTGCAAGTTCGACGAGGACGACAGCGAGCCCGTGCTGAAGGGCG | |
| | TGAAGCTGCACTACACCGGAAGCGGAGCCACGAACTTCTCTCTGTTAAAGC | |
| | AAGCAGGAGATGTTGAAGAAAACCCCGGGCCTATGAAGGCAATACTAGTAG | |
| | TTCTGCTATATACATTTGCAACCGCAAATGCAGACACATTATGTATAGGTTAT | |
| | CATGCGAACAACTCAACAGACACTGTAGACACAGTACTAGAAAAGAATGTAA | |
| | CAGTAACACACTCTGTTAACCTTCTAGAAGACAAGCATAACGGGAAACTATG | |
| | CAAACTAAGAGGGGTAGCCCCATTGCATTTGGGTAAATGTAACATTGCTGG | |
| | CTGGATCCTGGGAAATCCAGAGTGTGAATCACTCTCCACAGCAAGCTCATG | |
| | GTCCTACATTGTGGAAACATCTAGTTCAGACAATGGAACGTGTTACCCAGGA | |
| | GATTTCATCGATTATGAGGAGCTAAGAGAGCAATTGAGCTCAGTGTCATCAT | |
| | TTGAAAGGTTTGAGATATTCCCCAAGACAAGTTCATGGCCCAATCATGACTC | |
| | GAACAAAGGTGTAACGACAGCATGTCCTCATGCTGGAGCAAAAAGCTTCTA | |
| | CAAAAATTTAATATGGCTAGTTAAAAAAGGAAATTCATACCCAAAGCTCAGCA | |
| | AATCCTACATTAATGATAAAGGGAAAGAAGTCCTCGTGCTATGGGGCATTCA | |
| | CCATCCACCTACTAGTGCTGACCAACAAAGTCTCTATCAGAATGCAGATGCA | |
| | TATGTTTTTGTGGGGACATCAAGATACAGCAAGAAGTTCAAGCCGGAAATAG | |
| | CAATAAGGCCCAAAGTGAGGGATCAAGAAGGGAGAATGAACTATTACTGGA | |
| | CACTAGTAGAGCCGGGAGACAAAATAACATTCGAAGCAACTGGAAATCTAG | |
| | TGGTACCGAGATATGCATTCGCAATGGAAAGAAATGCTGGATCTGGTATTAT | |
| | CATTTCAGATACACCAGTCCACGATTGCAATACAACTTGTCAGACACCCAAG | |
| | GGTGCTATAAACACCAGCCTCCCATTTCAGAACATACATCCGATCACAATTG | |
| | GAAAATGTCCAAAATATGTAAAAAGCACAAAATTGAGACTGGCCACAGGATT | |
| | GAGGAATGTCCCGTCCATTCAATCTAGAGGCCTATTTGGGGCCATTGCCGG | |
| | TTTCATTGAAGGGGGGTGGACAGGGATGGTAGATGGATGGTACGGTTATCA | |
| | CCATCAAAATGAGCAGGGGTCAGGATATGCAGCCGACCTGAAGAGCACACA | |
| | GAATGCCATTGACGAGATTACTAACAAAGTAAACTCTGTTATTGAAAAAATGA | |
| | ATACACAGTTCACAGCAGTAGGTAAAGAGTTCAACCACCTGGAAAAAAGAAT | |
| | AGAGAATTTAAATAAAAAAGTTGATGATGGTTTCCTGGACATTTGGACTTACA | |
| | ATGCCGAACTGTTGGTTCTATTGGAAAATGAAAGAACTTTGGACTACCACGA | |
| | TTCAAAGGTGAAGAACTTATATGAAAAGGTAAGAAGCCAGTTAAAAAACAAT | |
| | GCCAAGGAAATTGGAAACGGCTGCTTTGAATTTTACCACAAATGTGATAACA | |
| | CGTGCATGGAAAGTGTCAAAAATGGGACTTATGACTACCCAAAATACTCAGA | |
| | GGAAGCAAAATTAAACAGAGAAGAAATAGATGGGGTAAAGCTGGAATCAAC | |
| | AAGGATTTACCAGATTTTGGCGATCTATTCAACCGTCGCCAGTTCATTGGTA | |
| | CTGGTAGTCTCCCTGGGGGCAATCAGTTTCTGGATGTGCTCTAATGGGTCT | |
| | CTACAGTGTAGAATATGTATTTAAGGAAGCGGAGCCACGAACTTCTCTCTGT | |
| | TAAAGCAAGCAGGAGATGTTGAAGAAAACCCCGGGCCTAGCAAAAGCAGG | |
| | GGATAATTCTATTAACCATGAAGACTATCATTGCTTTGAGCTACATTCTATGT | |
| | CTGGTTTTCGCTCAAAAACTTCCTGGAAATGACAATAGCACTGCAACGCTGT | |
| | GCCTTGGGCACCATGCAGTACCAAACGGAACGATAGTGAAAACAATCACGA | |
| | ATGACCAAATTGAAGTTACTAATGCTACTGAGCTGGTTCAGAATTCCTCAAT | |
| | AGGTGAAATATGCGACAGTCCTCATCAGATCCTTGATGGAGAAAACTGCAC | |
| | ACTAATAGATGCTCTATTGGGAGACCCTCAGTGTGATGGCTTTCAAAATAAG | |
| | AAATGGGACCTTTTTGTTGAACGAAGCAAAGCCTACAGCAACTGTTACCCTT | |
| | ATGATGTGCCGGATTATGCCTCCCTTAGGTCACTAGTTGCCTCATCCGGCA | |
| | CACTGGAGTTTAACAATGAAAGCTTCAATTGGGCTGGAGTCACTCAAAACG | |
| | GAACAAGTTCTGCTTGCATAAGGGGATCTAATAGTAGTTTCTTTAGTAGATTA | |
| | AATTGGTTGACCCACTTAAACTTCAAGTACCCAGCATTGAACGTGACTATGC | |
| | CAAACAATGAACAATTTGACAAATTGTACATTTGGGGGGTTCACCACCCGGG | |
| | TACGGACAAGGACCAAATCTTCCTGTATGCTCAATCATCAGGAAGAATCACA | |
| | GTATCTACCAAAAGAAGCCAACAAGCTGTAATCCCGAATATCGGATCTAGAC | |
| | CCAGAATAAGGAATATCCCTAGCAGAATAAGCATCTATTGGACAATAGTAAA | |
| | ACCGGGAGACATACTTTTGATTAACAGCACAGGGAATCTAATTGCTCCTAGG | |
| | GGTTACTTCAAAATACGAAGTGGGAAAAGCTCAATAATGAGATCAGATGCAC | |
| | CCATTGGCAAATGCAAGTCTGAATGCATCACTCCAAATGGAAGCATTCCCAA | |
| | TGACAAACCATTCCAAAATGTAAACAGGATCACATACGGGGCCTGTCCCAG | |
| | ATATGTTAAGCAAAGCACTCTGAAATTGGCAACAGGAATGCGAAATGTACCA | |
| | GAGAAACAAACTAGAGGCATATTTGGCGCAATAGCGGGTTTCATAGAAAAT | |
| | GGTTGGGAGGGAATGGTGGATGGTTGGTACGGTTTCAGGCATCAAAATTCT | |
| | GAGGGAAGAGGACAAGCAGCAGATCTCAAAAGCACTCAAGCAGCAATCGAT | |
| | CAAATCAATGGGAAGCTGAATCGATTGATCGGGAAAACCAACGAGAAATTC | |
| | CATCAGATTGAGAAAGAATTCTCAGAAGTAGAAGGGAGAATTCAGGACCTT | |
| | GAGAAATATGTTGAGGACACAAAAATAGATCTCTGGTCATACAACGCAGAGC | |
| | TTCTTGTTGCCCTGGAAAACCAACATACAATTGATCTAACTGACTCAGAAAT | |
| | GAACAAACTGTTTGAAAAAACAAAGAAGCAACTGAGGGAAAATGCTGAGGA | |
| | TATGGGCAATGGTTGTTTCAAAATATACCACAAATGTGACAATGCCTGCATA | |
| | GGATCAATCAGAAATGGAACTTATGACCACGATGTATACAGGGATGAAGCAT | |
| | TAAACAACCGGTTCCAGATCAAGGGAGTTGAGCTGAAGTCAGGGTACAAAG | |
| | ATTGGATCCTATGGATTTCCTTTGCCATATCATGTTTTTTGCTTTGTGTTGCT | |
| | TTGTTGGGGTTCATCATGTGGGCCTGCCAAAAGGGCAACATTAGGTGCAAC | |
| | ATTTGCATTTGAGTGCATTAATTAAAAACACCCTTGTTTCTACTGGAAGCGGA | |
| | GCCACGAACTTCTCTCTGTTAAAGCAAGCAGGAGATGTTGAAGAAAACCCC | |
| | GGGCCTATTTTCTAATATCCACAAAATGAAGGCAATAATTGTACTACTCATG | |
| | GTAGTAACATCCAATGCAGATCGAATCTGCACTGGGATAACATCGTCAAACT | |
| | CACCACATGTCGTCAAAACTGCTACTCAAGGGGAGGTCAACGTGACCGGTG | |
| | TAATACCACTGACAACAACACCCACCAAATCTCATTTTGCAAATCTCAAAGG | |
| | AACAGAAACCAGGGGGAAACTATGCCCAAAATGCCTCAACTGCACAGATCT | |
| | GGATGTAGCCTTGGGCAGACCAAAATGCACAGGGAAAATACCCTCTGCAAG | |
| | GGTTTCAATACTCCATGAAGTCAGACCTGTTACATCTGGGTGCTTTCCTATA | |
| | ATGCACGATAGAACAAAAATTAGACAGCTGCCTAACCTTCTCCGAGGATACG | |
| | AACATGTCAGGTTATCAACTCACAACGTTATCAATGCAAAAGATGCACCAGG | |
| | AAGACCCTACGAAATTGGAACCTCAGGGTCTTGCCCTAACATTACCAATGGA | |
| | AACGGATTCTTCGCAACAATGGCTTGGGCCGTCCCAAAAAACAAAACAGCA | |
| | ACAAATCCATTAACAATAGAAGTACCATACATTTGTACAGAAGGAGAAGACC | |
| | AAATTACCGTTTGGGGGTTCCACTCTGACAACGAGACCCAAATGGCAAAGC | |
| | TCTATGGGGACTCAAAGCCCCAGAAGTTCACCTCATCTGCCAACGGAGTGA | |
| | CCACACATTACGTTTCACAGATTGGTGGCTTCCCAAATCAAACAGAAGACGG | |
| | AGGACTACCACAAAGTGGCAGAATTGTTGTTGATTACATGGTGCAGAAATCT | |
| | GGAAAAACAGGAACAATTACCTATCAAAGAGGTATTTTATTGCCTCAAAAAG | |
| | TGTGGTGCGCAAGTGGCAGGAGCAAGGTAATAAAAGGATCCTTGCCCTTAA | |
| | TTGGAGAAGCAGATTGCCTCCATGAAAAATACGGTGGATTAAACAAAAGCAA | |
| | GCCTTACTACACAGGGGAACATGCAAAGGCCATAGGAAATTGCCCAATATG | |
| | GGTGAAAACACCCTTGAAGCTGGCCAATGGAACCAAATATAGACCCCCTGC | |
| | AAAACTATTAAAGGAAAGAGGTTTCTTCGGAGCCATTGCTGGTTTCTTAGAG | |
| | GGAGGATGGGAAGGAATGATTGCAGGTTGGCACGGATACACATCCCATGG | |
| | GGCACATGGAGTAGCGGTGGCAGCTGACCTTAAGAGCACTCAAGAGGCCA | |
| | TAAACAAGATAACAAAAAATCTCAACTCTTTGAGTGAGCTGGAAGTAAAGAA | |
| | TCTTCAAAGACTAAGCGGTGCCATGGATGAACTCCACAACGAAATACTAGAA | |
| | CTAGATGAGAAAGTGGATGATCTCAGAGCTGACACAATAAGCTCACAAATAG | |
| | AACTCGCAGTCCTGCTTTCCAATGAAGGAATAATAAACAGTGAAGATGAACA | |
| | TCTCTTGGCGCTTGAAAGAAAGCTGAAGAAAATGCTGGGCCCCTCTGCTGT | |
| | AGAGATAGGGAATGGATGCTTTGAAACCAAACACAAGTGCAACCAGACCTG | |
| | CCTCGACAGAATAGCTGCTGGTACCTTTGATGCAGGAGAATTTTCTCTCCCC | |
| | ACCTTTGATTCACTGAATATTACTGCTGCATCTTTAAATGACGACGGATTGG | |
| | ACAATCATACTATACTGCTTTACTACTCAACTGCTGCCTCCAGTTTGGCTGTA | |
| | ACACTGATGATAGCTATCTTTGTTGTTTATATGGTCTCCAGAGACAATGTTTC | |
| | TTGCTCCATTTGTCTATAAGGAAAGTTAAGCCCTGTATTTTCCTTTATTGTAG | |
| | TGCTTGTTTGCTTGTTGTCATTACAAAGAAACGTTATTGAAAAATTGATGACT | |
| | CGAGCTGGTACTGCATGCACGCAATGCTAGCTGCCCCTTTCCCGTCCTGGG | |
| | TACCCCGAGTCTCCCCCGACCTCGGGTCCCAGGTATGCTCCCACCTCCACC | |
| | TGCCCCACTCACCACCTCTGCTAGTTCCAGACACCTCCCAAGCACGCAGCA | |
| | ATGCAGCTCAAAACGCTTAGCCTAGCCACACCCCCACGGGAAACAGCAGTG | |
| | ATTAACCTTTAGCAATAAACGAAAGTTTAACTAAGCTATACTAACCCCAGGGT | |
| | TGGTCAATTTCGTGCCAGCCACACCCTGGAGCTAGCAAAAAAAA | |
| Pan-CoV- | CTCGACATTGATTATTGACTAGTTATTAATAGTAATCAATTACGGGGTCATTA | 37 |
| Flu | GTTCATAGCCCATATATGGAGTTCCGCGTTACATAACTTACGGTAAATGGCC | |
| Vaccine | CGCCTGGCTGACCGCCCAACGACCCCCGCCCATTGACGTCAATAATGACGT | |
| #17 CoV- | ATGTTCCCATAGTAACGCCAATAGGGACTTTCCATTGACGTCAATGGGTGGA | |
| Omicron | GTATTTACGGTAAACTGCCCACTTGGCAGTACATCAAGTGTATCATATGCCA | |
| BA.2 Spike | AGTACGCCCCCTATTGACGTCAATGACGGTAAATGGCCCGCCTGGCATTAT | |
| (HexaPro)- | GCCCAGTACATGACCTTATGGGACTTTCCTACTTGGCAGTACATCTACGTAT | |
| Influenza | TAGTCATCGCTATTACCATGGTCGAGGTGAGCCCCACGTTCTGCTTCACTCT | |
| (HA) | CCCCATCTCCCCCCCCTCCCCACCCCCAATTTTGTATTTATTTATTTTTTAAT | |
| | TATTTTGTGCAGCGATGGGGGCGGGGGGGGGGGGGGGGCGCGCGCCAG | |
| | GCGGGGCGGGGCGGGGCGAGGGGCGGGGCGGGGCGAGGCGGAGAGGT | |
| | GCGGCGGCAGCCAATCAGAGCGGCGCGCTCCGAAAGTTTCCTTTTATGGC | |
| | GAGGCGGCGGCGGCGGCGGCCCTATAAAAAGCGAAGCGCGCGGGGGC | |
| | GGGAGTCGCTGCGCGCTGCCTTCGCCCCGTGCCCCGCTCCGCCGCCGCC | |
| | TCGCGCCGCCCGCCCCGGCTCTGACTGACCGCGTTACTCCCACAGGTGAG | |
| | CGGGCGGGACGGCCCTTCTCCTCCGGGCTGTAATTAGCGCTTGGTTTAATG | |
| | ACGGCTTGTTTCTTTTCTGTGGCTGCGTGAAAGCCTTGAGGGGCTCCGGGA | |
| | GGGCCCTTTGTGCGGGGGGAGCGGCTCGGGGGGTGCGTGCGTGTGTGTG | |
| | TGCGTGGGGAGCGCCGCGTGCGGCTCCGCGCTGCCCGGCGGCTGTGAGC | |
| | GCTGCGGGCGCGGCGCGGGGCTTTGTGCGCTCCGCAGTGTGCGCGAGGG | |
| | GAGCGCGGCCGGGGGGGGTGCCCCGCGGTGCGGGGGGGGCTGCGAGGG | |
| | GAACAAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCCCGAGT | |
| | TGCTGAGCACGGCCCGGCTTCGGGTGCGGGGCTCCGTACGGGGCGTGGC | |
| | GCGGGGCTCGCCGTGCCGGGGGGGGGGGGCGGCAGGTGGGGGTGCCG | |
| | GGCGGGGGGGGGCCGCCTCGGGCCGGGGAGGGCTCGGGGGAGGGGCG | |
| | CGGCGGCCCCCGGAGCGCCGGCGGCTGTCGAGGCGCGGCGAGCCGCAG | |
| | CCATTGCCTTTTATGGTAATCGTGCGAGAGGGCGCAGGGACTTCCTTTGTC | |
| | CCAAATCTGTGCGGAGCCGAAATCTGGGAGGCGCCGCCGCACCCCCTCTA | |
| | GCGGGCGCGGGGCGAAGCGGTGCGGCGCCGGCAGGAAGGAAATGGGCG | |
| | GGGAGGGCCTTCGTGCGTCGCCGCGCCGCCGTCCCCTTCTCCCTCTCCAG | |
| | CCTCGGGGCTGTCCGGGGGGGGACGGCTGCCTTCGGGGGGGACGGGGC | |
| | AGGGGGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGTGCTG | |
| | GTTATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTTCCT | |
| | GGTGCTGCTGCCCCTGGTGAGCAGCCAGTGCGTGAACCTGATCACCAGGA | |
| | CCCAGAGCTACACCAACAGCTTCACCAGGGGCGTGTACTACCCCGACAAG | |
| | GTGTTCAGGAGCAGCGTGCTGCACAGCACCCAGGACCTGTTCCTGCCCTTC | |
| | TTCAGCAACGTGACCTGGTTCCACGCCATCCACGTGAGCGGCACCAACGG | |
| | CACCAAGAGGTTCGACAACCCCGTGCTGCCCTTCAACGACGGCGTGTACTT | |
| | CGCCAGCACCGAGAAGAGCAACATCATCAGGGGCTGGATCTTCGGCACCA | |
| | CCCTGGACAGCAAGACCCAGAGCCTGCTGATCGTGAACAACGCCACCAAC | |
| | GTGGTGATCAAGGTGTGCGAGTTCCAGTTCTGCAACGACCCCTTCCTGGAC | |
| | GTGTACTACCACAAGAACAACAAGAGCTGGATGGAGAGCGAGTTCAGGGTG | |
| | TACAGCAGCGCCAACAACTGCACCTTCGAGTACGTGAGCCAGCCCTTCCTG | |
| | ATGGACCTGGAGGGCAAGCAGGGCAACTTCAAGAACCTGAGGGAGTTCGT | |
| | GTTCAAGAACATCGACGGCTACTTCAAGATCTACAGCAAGCACACCCCCAT | |
| | CAACCTGGGCAGGGACCTGCCCCAGGGCTTCAGCGCCCTGGAGCCCCTG | |
| | GTGGACCTGCCCATCGGCATCAACATCACCAGGTTCCAGACCCTGCTGGCC | |
| | CTGCACAGGAGCTACCTGACCCCCGGCGACAGCAGCAGCGGCTGGACCG | |
| | CCGGCGCCGCCGCCTACTACGTGGGCTACCTGCAGCCCAGGACCTTCCTG | |
| | CTGAAGTACAACGAGAACGGCACCATCACCGACGCCGTGGACTGCGCCCT | |
| | GGACCCCCTGAGCGAGACCAAGTGCACCCTGAAGAGCTTCACCGTGGAGA | |
| | AGGGCATCTACCAGACCAGCAACTTCAGGGTGCAGCCCACCGAGAGCATC | |
| | GTGAGGTTCCCCAACATCACCAACCTGTGCCCCTTCGACGAGGTGTTCAAC | |
| | GCCACCAGGTTCGCCAGCGTGTACGCCTGGAACAGGAAGAGGATCAGCAA | |
| | CTGCGTGGCCGACTACAGCGTGCTGTACAACTTCGCCCCCTTCTTCGCCTT | |
| | CAAGTGCTACGGCGTGAGCCCCACCAAGCTGAACGACCTGTGCTTCACCAA | |
| | CGTGTACGCCGACAGCTTCGTGATCAGGGGCAACGAGGTGAGCCAGATCG | |
| | CCCCCGGCCAGACCGGCAACATCGCCGACTACAACTACAAGCTGCCCGAC | |
| | GACTTCACCGGCTGCGTGATCGCCTGGAACAGCAACAAGCTGGACAGCAA | |
| | GGTGGGCGGCAACTACAACTACCTGTACAGGCTGTTCAGGAAGAGCAACCT | |
| | GAAGCCCTTCGAGAGGGACATCAGCACCGAGATCTACCAGGCCGGCAACA | |
| | AGCCCTGCAACGGCGTGGCCGGCTTCAACTGCTACTTCCCCCTGAGGAGC | |
| | TACGGCTTCAGGCCCACCTACGGCGTGGGCCACCAGCCCTACAGGGTGGT | |
| | GGTGCTGAGCTTCGAGCTGCTGCACGCCCCCGCCACCGTGTGCGGCCCCA | |
| | AGAAGAGCACCAACCTGGTGAAGAACAAGTGCGTGAACTTCAACTTCAACG | |
| | GCCTGACCGGCACCGGCGTGCTGACCGAGAGCAACAAGAAGTTCCTGCCC | |
| | TTCCAGCAGTTCGGCAGGGACATCGCCGACACCACCGACGCCGTGAGGGA | |
| | CCCCCAGACCCTGGAGATCCTGGACATCACCCCCTGCAGCTTCGGCGGCG | |
| | TGAGCGTGATCACCCCCGGCACCAACACCAGCAACCAGGTGGCCGTGCTG | |
| | TACCAGGGCGTGAACTGCACCGAGGTGCCCGTGGCCATCCACGCCGACCA | |
| | GCTGACCCCCACCTGGAGGGTGTACAGCACCGGCAGCAACGTGTTCCAGA | |
| | CCAGGGCCGGCTGCCTGATCGGCGCCGAGTACGTGAACAACAGCTACGAG | |
| | TGCGACATCCCCATCGGCGCCGGCATCTGCGCCAGCTACCAGACCCAGAC | |
| | CAAGAGCCACCAGCAGGCCCAGAGCGTGGCCAGCCAGAGCATCATCGCCT | |
| | ACACCATGAGCCTGGGCGCCGAGAACAGCGTGGCCTACAGCAACAACAGC | |
| | ATCGCCATCCCCACCAACTTCACCATCAGCGTGACCACCGAGATCCTGCCC | |
| | GTGAGCATGACCAAGACCAGCGTGGACTGCACCATGTACATCTGCGGCGA | |
| | CAGCACCGAGTGCAGCAACCTGCTGCTGCAGTACGGCAGCTTCTGCACCC | |
| | AGCTGAAGAGGGCCCTGACCGGCATCGCCGTGGAGCAGGACAAGAACACC | |
| | CAGGAGGTGTTCGCCCAGGTGAAGCAGATCTACAAGACCCCCCCCATCAA | |
| | GTACTTCGGCGGCTTCAACTTCAGCCAGATCCTGCCCGACCCCAGCAAGCC | |
| | CAGCAAGAGGAGCCCCATCGAGGACCTGCTGTTCAACAAGGTGACCCTGG | |
| | CCGACGCCGGCTTCATCAAGCAGTACGGCGACTGCCTGGGCGACATCGCC | |
| | GCCAGGGACCTGATCTGCGCCCAGAAGTTCAACGGCCTGACCGTGCTGCC | |
| | CCCCCTGCTGACCGACGAGATGATCGCCCAGTACACCAGCGCCCTGCTGG | |
| | CCGGCACCATCACCAGCGGCTGGACCTTCGGCGCCGGCCCCGCCCTGCA | |
| | GATCCCCTTCCCCATGCAGATGGCCTACAGGTTCAACGGCATCGGCGTGAC | |
| | CCAGAACGTGCTGTACGAGAACCAGAAGCTGATCGCCAACCAGTTCAACAG | |
| | CGCCATCGGCAAGATCCAGGACAGCCTGAGCAGCACCCCCAGCGCCCTGG | |
| | GCAAGCTGCAGGACGTGGTGAACCACAACGCCCAGGCCCTGAACACCCTG | |
| | GTGAAGCAGCTGAGCAGCAAGTTCGGCGCCATCAGCAGCGTGCTGAACGA | |
| | CATCCTGAGCAGGCTGGACCCCCCCGAGGCCGAGGTGCAGATCGACAGGC | |
| | TGATCACCGGCAGGCTGCAGAGCCTGCAGACCTACGTGACCCAGCAGCTG | |
| | ATCAGGGCCGCCGAGATCAGGGCCAGCGCCAACCTGGCCGCCACCAAGAT | |
| | GAGCGAGTGCGTGCTGGGCCAGAGCAAGAGGGTGGACTTCTGCGGCAAG | |
| | GGCTACCACCTGATGAGCTTCCCCCAGAGCGCCCCCCACGGCGTGGTGTT | |
| | CCTGCACGTGACCTACGTGCCCGCCCAGGAGAAGAACTTCACCACCGCCC | |
| | CCGCCATCTGCCACGACGGCAAGGCCCACTTCCCCAGGGAGGGCGTGTTC | |
| | GTGAGCAACGGCACCCACTGGTTCGTGACCCAGAGGAACTTCTACGAGCC | |
| | CCAGATCATCACCACCGACAACACCTTCGTGAGCGGCAACTGCGACGTGGT | |
| | GATCGGCATCGTGAACAACACCGTGTACGACCCCCTGCAGCCCGAGCTGG | |
| | ACAGCTTCAAGGAGGAGCTGGACAAGTACTTCAAGAACCACACCAGCCCCG | |
| | ACGTGGACCTGGGCGACATCAGCGGCATCAACGCCAGCGTGGTGAACATC | |
| | CAGAAGGAGATCGACAGGCTGAACGAGGTGGCCAAGAACCTGAACGAGAG | |
| | CCTGATCGACCTGCAGGAGCTGGGCAAGTACGAGCAGTACATCAAGTGGC | |
| | CCTGGTACATCTGGCTGGGCTTCATCGCCGGCCTGATCGCCATCGTGATGG | |
| | TGACCATCATGCTGTGCTGCATGACCAGCTGCTGCAGCTGCCTGAAGGGCT | |
| | GCTGCAGCTGCGGCAGCTGCTGCAAGTTCGACGAGGACGACAGCGAGCCC | |
| | GTGCTGAAGGGCGTGAAGCTGCACTACACCGGAAGCGGAGCCACGAACTT | |
| | CTCTCTGTTAAAGCAAGCAGGAGATGTTGAAGAAAACCCCGGGCCTATGAA | |
| | GGCAATACTAGTAGTTCTGCTATATACATTTGCAACCGCAAATGCAGACACA | |
| | TTATGTATAGGTTATCATGCGAACAACTCAACAGACACTGTAGACACAGTAC | |
| | TAGAAAAGAATGTAACAGTAACACACTCTGTTAACCTTCTAGAAGACAAGCA | |
| | TAACGGGAAACTATGCAAACTAAGAGGGGTAGCCCCATTGCATTTGGGTAA | |
| | ATGTAACATTGCTGGCTGGATCCTGGGAAATCCAGAGTGTGAATCACTCTC | |
| | CACAGCAAGCTCATGGTCCTACATTGTGGAAACATCTAGTTCAGACAATGGA | |
| | ACGTGTTACCCAGGAGATTTCATCGATTATGAGGAGCTAAGAGAGCAATTGA | |
| | GCTCAGTGTCATCATTTGAAAGGTTTGAGATATTCCCCAAGACAAGTTCATG | |
| | GCCCAATCATGACTCGAACAAAGGTGTAACGACAGCATGTCCTCATGCTGG | |
| | AGCAAAAAGCTTCTACAAAAATTTAATATGGCTAGTTAAAAAAGGAAATTCAT | |
| | ACCCAAAGCTCAGCAAATCCTACATTAATGATAAAGGGAAAGAAGTCCTCGT | |
| | GCTATGGGGCATTCACCATCCACCTACTAGTGCTGACCAACAAAGTCTCTAT | |
| | CAGAATGCAGATGCATATGTTTTTGTGGGGACATCAAGATACAGCAAGAAGT | |
| | TCAAGCCGGAAATAGCAATAAGGCCCAAAGTGAGGGATCAAGAAGGGAGAA | |
| | TGAACTATTACTGGACACTAGTAGAGCCGGGAGACAAAATAACATTCGAAG | |
| | CAACTGGAAATCTAGTGGTACCGAGATATGCATTCGCAATGGAAAGAAATG | |
| | CTGGATCTGGTATTATCATTTCAGATACACCAGTCCACGATTGCAATACAAC | |
| | TTGTCAGACACCCAAGGGTGCTATAAACACCAGCCTCCCATTTCAGAACATA | |
| | CATCCGATCACAATTGGAAAATGTCCAAAATATGTAAAAAGCACAAAATTGA | |
| | GACTGGCCACAGGATTGAGGAATGTCCCGTCCATTCAATCTAGAGGCCTAT | |
| | TTGGGGCCATTGCCGGTTTCATTGAAGGGGGGTGGACAGGGATGGTAGAT | |
| | GGATGGTACGGTTATCACCATCAAAATGAGCAGGGGTCAGGATATGCAGCC | |
| | GACCTGAAGAGCACACAGAATGCCATTGACGAGATTACTAACAAAGTAAACT | |
| | CTGTTATTGAAAAAATGAATACACAGTTCACAGCAGTAGGTAAAGAGTTCAA | |
| | CCACCTGGAAAAAAGAATAGAGAATTTAAATAAAAAAGTTGATGATGGTTTC | |
| | CTGGACATTTGGACTTACAATGCCGAACTGTTGGTTCTATTGGAAAATGAAA | |
| | GAACTTTGGACTACCACGATTCAAAGGTGAAGAACTTATATGAAAAGGTAAG | |
| | AAGCCAGTTAAAAAACAATGCCAAGGAAATTGGAAACGGCTGCTTTGAATTT | |
| | TACCACAAATGTGATAACACGTGCATGGAAAGTGTCAAAAATGGGACTTATG | |
| | ACTACCCAAAATACTCAGAGGAAGCAAAATTAAACAGAGAAGAAATAGATGG | |
| | GGTAAAGCTGGAATCAACAAGGATTTACCAGATTTTGGCGATCTATTCAACC | |
| | GTCGCCAGTTCATTGGTACTGGTAGTCTCCCTGGGGGCAATCAGTTTCTGG | |
| | ATGTGCTCTAATGGGTCTCTACAGTGTAGAATATGTATTTAAGGAAGCGGAG | |
| | CCACGAACTTCTCTCTGTTAAAGCAAGCAGGAGATGTTGAAGAAAACCCCG | |
| | GGCCTAGCAAAAGCAGGGGATAATTCTATTAACCATGAAGACTATCATTGCT | |
| | TTGAGCTACATTCTATGTCTGGTTTTCGCTCAAAAACTTCCTGGAAATGACAA | |
| | TAGCACTGCAACGCTGTGCCTTGGGCACCATGCAGTACCAAACGGAACGAT | |
| | AGTGAAAACAATCACGAATGACCAAATTGAAGTTACTAATGCTACTGAGCTG | |
| | GTTCAGAATTCCTCAATAGGTGAAATATGCGACAGTCCTCATCAGATCCTTG | |
| | ATGGAGAAAACTGCACACTAATAGATGCTCTATTGGGAGACCCTCAGTGTG | |
| | ATGGCTTTCAAAATAAGAAATGGGACCTTTTTGTTGAACGAAGCAAAGCCTA | |
| | CAGCAACTGTTACCCTTATGATGTGCCGGATTATGCCTCCCTTAGGTCACTA | |
| | GTTGCCTCATCCGGCACACTGGAGTTTAACAATGAAAGCTTCAATTGGGCT | |
| | GGAGTCACTCAAAACGGAACAAGTTCTGCTTGCATAAGGGGATCTAATAGTA | |
| | GTTTCTTTAGTAGATTAAATTGGTTGACCCACTTAAACTTCAAGTACCCAGCA | |
| | TTGAACGTGACTATGCCAAACAATGAACAATTTGACAAATTGTACATTTGGG | |
| | GGGTTCACCACCCGGGTACGGACAAGGACCAAATCTTCCTGTATGCTCAAT | |
| | CATCAGGAAGAATCACAGTATCTACCAAAAGAAGCCAACAAGCTGTAATCCC | |
| | GAATATCGGATCTAGACCCAGAATAAGGAATATCCCTAGCAGAATAAGCATC | |
| | TATTGGACAATAGTAAAACCGGGAGACATACTTTTGATTAACAGCACAGGGA | |
| | ATCTAATTGCTCCTAGGGGTTACTTCAAAATACGAAGTGGGAAAAGCTCAAT | |
| | AATGAGATCAGATGCACCCATTGGCAAATGCAAGTCTGAATGCATCACTCCA | |
| | AATGGAAGCATTCCCAATGACAAACCATTCCAAAATGTAAACAGGATCACAT | |
| | ACGGGGCCTGTCCCAGATATGTTAAGCAAAGCACTCTGAAATTGGCAACAG | |
| | GAATGCGAAATGTACCAGAGAAACAAACTAGAGGCATATTTGGCGCAATAG | |
| | CGGGTTTCATAGAAAATGGTTGGGAGGGAATGGTGGATGGTTGGTACGGTT | |
| | TCAGGCATCAAAATTCTGAGGGAAGAGGACAAGCAGCAGATCTCAAAAGCA | |
| | CTCAAGCAGCAATCGATCAAATCAATGGGAAGCTGAATCGATTGATCGGGA | |
| | AAACCAACGAGAAATTCCATCAGATTGAGAAAGAATTCTCAGAAGTAGAAGG | |
| | GAGAATTCAGGACCTTGAGAAATATGTTGAGGACACAAAAATAGATCTCTGG | |
| | TCATACAACGCAGAGCTTCTTGTTGCCCTGGAAAACCAACATACAATTGATC | |
| | TAACTGACTCAGAAATGAACAAACTGTTTGAAAAAACAAAGAAGCAACTGAG | |
| | GGAAAATGCTGAGGATATGGGCAATGGTTGTTTCAAAATATACCACAAATGT | |
| | GACAATGCCTGCATAGGATCAATCAGAAATGGAACTTATGACCACGATGTAT | |
| | ACAGGGATGAAGCATTAAACAACCGGTTCCAGATCAAGGGAGTTGAGCTGA | |
| | AGTCAGGGTACAAAGATTGGATCCTATGGATTTCCTTTGCCATATCATGTTTT | |
| | TTGCTTTGTGTTGCTTTGTTGGGGTTCATCATGTGGGCCTGCCAAAAGGGC | |
| | AACATTAGGTGCAACATTTGCATTTGAGTGCATTAATTAAAAACACCCTTGTT | |
| | TCTACTGGAAGCGGAGCCACGAACTTCTCTCTGTTAAAGCAAGCAGGAGAT | |
| | GTTGAAGAAAACCCCGGGCCTATTTTCTAATATCCACAAAATGAAGGCAATA | |
| | ATTGTACTACTCATGGTAGTAACATCCAATGCAGATCGAATCTGCACTGGGA | |
| | TAACATCGTCAAACTCACCACATGTCGTCAAAACTGCTACTCAAGGGGAGGT | |
| | CAACGTGACCGGTGTAATACCACTGACAACAACACCCACCAAATCTCATTTT | |
| | GCAAATCTCAAAGGAACAGAAACCAGGGGGAAACTATGCCCAAAATGCCTC | |
| | AACTGCACAGATCTGGATGTAGCCTTGGGCAGACCAAAATGCACAGGGAAA | |
| | ATACCCTCTGCAAGGGTTTCAATACTCCATGAAGTCAGACCTGTTACATCTG | |
| | GGTGCTTTCCTATAATGCACGATAGAACAAAAATTAGACAGCTGCCTAACCT | |
| | TCTCCGAGGATACGAACATGTCAGGTTATCAACTCACAACGTTATCAATGCA | |
| | AAAGATGCACCAGGAAGACCCTACGAAATTGGAACCTCAGGGTCTTGCCCT | |
| | AACATTACCAATGGAAACGGATTCTTCGCAACAATGGCTTGGGCCGTCCCA | |
| | AAAAAAAAACAGCAACAAATCCATTAACAATAGAAGTACCATACATTTGTAC | |
| | AGAAGGAGAAGACCAAATTACCGTTTGGGGGTTCCACTCTGACAACGAGAC | |
| | CCAAATGGCAAAGCTCTATGGGGACTCAAAGCCCCAGAAGTTCACCTCATC | |
| | TGCCAACGGAGTGACCACACATTACGTTTCACAGATTGGTGGCTTCCCAAAT | |
| | CAAACAGAAGACGGAGGACTACCACAAAGTGGCAGAATTGTTGTTGATTAC | |
| | ATGGTGCAGAAATCTGGAAAAACAGGAACAATTACCTATCAAAGAGGTATTT | |
| | TATTGCCTCAAAAAGTGTGGTGCGCAAGTGGCAGGAGCAAGGTAATAAAAG | |
| | GATCCTTGCCCTTAATTGGAGAAGCAGATTGCCTCCATGAAAAATACGGTG | |
| | GATTAAACAAAAGCAAGCCTTACTACACAGGGGAACATGCAAAGGCCATAG | |
| | GAAATTGCCCAATATGGGTGAAAACACCCTTGAAGCTGGCCAATGGAACCA | |
| | AATATAGACCCCCTGCAAAACTATTAAAGGAAAGAGGTTTCTTCGGAGCCAT | |
| | TGCTGGTTTCTTAGAGGGAGGATGGGAAGGAATGATTGCAGGTTGGCACG | |
| | GATACACATCCCATGGGGCACATGGAGTAGCGGTGGCAGCTGACCTTAAGA | |
| | GCACTCAAGAGGCCATAAACAAGATAACAAAAAATCTCAACTCTTTGAGTGA | |
| | GCTGGAAGTAAAGAATCTTCAAAGACTAAGCGGTGCCATGGATGAACTCCA | |
| | CAACGAAATACTAGAACTAGATGAGAAAGTGGATGATCTCAGAGCTGACAC | |
| | AATAAGCTCACAAATAGAACTCGCAGTCCTGCTTTCCAATGAAGGAATAATA | |
| | AACAGTGAAGATGAACATCTCTTGGCGCTTGAAAGAAAGCTGAAGAAAATG | |
| | CTGGGCCCCTCTGCTGTAGAGATAGGGAATGGATGCTTTGAAACCAAACAC | |
| | AAGTGCAACCAGACCTGCCTCGACAGAATAGCTGCTGGTACCTTTGATGCA | |
| | GGAGAATTTTCTCTCCCCACCTTTGATTCACTGAATATTACTGCTGCATCTTT | |
| | AAATGACGACGGATTGGACAATCATACTATACTGCTTTACTACTCAACTGCT | |
| | GCCTCCAGTTTGGCTGTAACACTGATGATAGCTATCTTTGTTGTTTATATGGT | |
| | CTCCAGAGACAATGTTTCTTGCTCCATTTGTCTATAAGGAAAGTTAAGCCCT | |
| | GTATTTTCCTTTATTGTAGTGCTTGTTTGCTTGTTGTCATTACAAAGAAACGT | |
| | TATTGAAAAATTGATGACTCGAGCTGGTACTGCATGCACGCAATGCTAGCTG | |
| | CCCCTTTCCCGTCCTGGGTACCCCGAGTCTCCCCCGACCTCGGGTCCCAG | |
| | GTATGCTCCCACCTCCACCTGCCCCACTCACCACCTCTGCTAGTTCCAGAC | |
| | ACCTCCCAAGCACGCAGCAATGCAGCTCAAAACGCTTAGCCTAGCCACACC | |
| | CCCACGGGAAACAGCAGTGATTAACCTTTAGCAATAAACGAAAGTTTAACTA | |
| | AGCTATACTAACCCCAGGGTTGGTCAATTTCGTGCCAGCCACACCCTGGAG | |
| | CTAGCAAAAAAAA | |
| Pan-CoV- | CTCGACATTGATTATTGACTAGTTATTAATAGTAATCAATTACGGGGTCATTA | 38 |
| Flu | GTTCATAGCCCATATATGGAGTTCCGCGTTACATAACTTACGGTAAATGGCC | |
| Vaccine | CGCCTGGCTGACCGCCCAACGACCCCCGCCCATTGACGTCAATAATGACGT | |
| #18 CoV- | ATGTTCCCATAGTAACGCCAATAGGGACTTTCCATTGACGTCAATGGGTGGA | |
| Omicron | GTATTTACGGTAAACTGCCCACTTGGCAGTACATCAAGTGTATCATATGCCA | |
| BA.2 Spike | AGTACGCCCCCTATTGACGTCAATGACGGTAAATGGCCCGCCTGGCATTAT | |
| (2 proline | GCCCAGTACATGACCTTATGGGACTTTCCTACTTGGCAGTACATCTACGTAT | |
| subst.)- | TAGTCATCGCTATTACCATGGTCGAGGTGAGCCCCACGTTCTGCTTCACTCT | |
| Influenza | CCCCATCTCCCCCCCCTCCCCACCCCCAATTTTGTATTTATTTATTTTTTAAT | |
| (HA) | TATTTTGTGCAGCGATGGGGGCGGGGGGGGGGGGGGGGCGCGCGCCAG | |
| | GCGGGGCGGGGCGGGGCGAGGGGCGGGGCGGGGCGAGGCGGAGAGGT | |
| | GCGGCGGCAGCCAATCAGAGCGGCGCGCTCCGAAAGTTTCCTTTTATGGC | |
| | GAGGCGGCGGCGGCGGCGGCCCTATAAAAAGCGAAGCGCGCGGCGGGC | |
| | GGGAGTCGCTGCGCGCTGCCTTCGCCCCGTGCCCCGCTCCGCCGCCGCC | |
| | TCGCGCCGCCCGCCCCGGCTCTGACTGACCGCGTTACTCCCACAGGTGAG | |
| | CGGGCGGGACGGCCCTTCTCCTCCGGGCTGTAATTAGCGCTTGGTTTAATG | |
| | ACGGCTTGTTTCTTTTCTGTGGCTGCGTGAAAGCCTTGAGGGGCTCCGGGA | |
| | GGGCCCTTTGTGCGGGGGGAGCGGCTCGGGGGGTGCGTGCGTGTGTGTG | |
| | TGCGTGGGGAGCGCCGCGTGCGGCTCCGCGCTGCCCGGCGGCTGTGAGC | |
| | GCTGCGGGCGCGGCGCGGGGCTTTGTGCGCTCCGCAGTGTGCGCGAGGG | |
| | GAGCGCGGCCGGGGGGGGTGCCCCGCGGTGCGGGGGGGGCTGCGAGGG | |
| | GAACAAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCCCGAGT | |
| | TGCTGAGCACGGCCCGGCTTCGGGTGCGGGGCTCCGTACGGGGCGTGGC | |
| | GCGGGGCTCGCCGTGCCGGCGGGGGGTGGCGGCAGGTGGGGGTGCCG | |
| | GGCGGGGCGGGGCCGCCTCGGGCCGGGGAGGGCTCGGGGGAGGGGCG | |
| | CGGCGGCCCCCGGAGCGCCGGCGGCTGTCGAGGCGCGGCGAGCCGCAG | |
| | CCATTGCCTTTTATGGTAATCGTGCGAGAGGGCGCAGGGACTTCCTTTGTC | |
| | CCAAATCTGTGCGGAGCCGAAATCTGGGAGGCGCCGCCGCACCCCCTCTA | |
| | GCGGGCGCGGGGCGAAGCGGTGCGGCGCCGGCAGGAAGGAAATGGGCG | |
| | GGGAGGGCCTTCGTGCGTCGCCGCGCCGCCGTCCCCTTCTCCCTCTCCAG | |
| | CCTCGGGGCTGTCCGCGGGGGGACGGCTGCCTTCGGGGGGGACGGGGC | |
| | AGGGGGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGTGCTG | |
| | GTTATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTTCCT | |
| | GGTGCTGCTGCCCCTGGTGAGCAGCCAGTGCGTGAACCTGATCACCAGGA | |
| | CCCAGAGCTACACCAACAGCTTCACCAGGGGCGTGTACTACCCCGACAAG | |
| | GTGTTCAGGAGCAGCGTGCTGCACAGCACCCAGGACCTGTTCCTGCCCTTC | |
| | TTCAGCAACGTGACCTGGTTCCACGCCATCCACGTGAGCGGCACCAACGG | |
| | CACCAAGAGGTTCGACAACCCCGTGCTGCCCTTCAACGACGGCGTGTACTT | |
| | CGCCAGCACCGAGAAGAGCAACATCATCAGGGGCTGGATCTTCGGCACCA | |
| | CCCTGGACAGCAAGACCCAGAGCCTGCTGATCGTGAACAACGCCACCAAC | |
| | GTGGTGATCAAGGTGTGCGAGTTCCAGTTCTGCAACGACCCCTTCCTGGAC | |
| | GTGTACTACCACAAGAACAACAAGAGCTGGATGGAGAGCGAGTTCAGGGTG | |
| | TACAGCAGCGCCAACAACTGCACCTTCGAGTACGTGAGCCAGCCCTTCCTG | |
| | ATGGACCTGGAGGGCAAGCAGGGCAACTTCAAGAACCTGAGGGAGTTCGT | |
| | GTTCAAGAACATCGACGGCTACTTCAAGATCTACAGCAAGCACACCCCCAT | |
| | CAACCTGGGCAGGGACCTGCCCCAGGGCTTCAGCGCCCTGGAGCCCCTG | |
| | GTGGACCTGCCCATCGGCATCAACATCACCAGGTTCCAGACCCTGCTGGCC | |
| | CTGCACAGGAGCTACCTGACCCCCGGCGACAGCAGCAGCGGCTGGACCG | |
| | CCGGCGCCGCCGCCTACTACGTGGGCTACCTGCAGCCCAGGACCTTCCTG | |
| | CTGAAGTACAACGAGAACGGCACCATCACCGACGCCGTGGACTGCGCCCT | |
| | GGACCCCCTGAGCGAGACCAAGTGCACCCTGAAGAGCTTCACCGTGGAGA | |
| | AGGGCATCTACCAGACCAGCAACTTCAGGGTGCAGCCCACCGAGAGCATC | |
| | GTGAGGTTCCCCAACATCACCAACCTGTGCCCCTTCGACGAGGTGTTCAAC | |
| | GCCACCAGGTTCGCCAGCGTGTACGCCTGGAACAGGAAGAGGATCAGCAA | |
| | CTGCGTGGCCGACTACAGCGTGCTGTACAACTTCGCCCCCTTCTTCGCCTT | |
| | CAAGTGCTACGGCGTGAGCCCCACCAAGCTGAACGACCTGTGCTTCACCAA | |
| | CGTGTACGCCGACAGCTTCGTGATCAGGGGCAACGAGGTGAGCCAGATCG | |
| | CCCCCGGCCAGACCGGCAACATCGCCGACTACAACTACAAGCTGCCCGAC | |
| | GACTTCACCGGCTGCGTGATCGCCTGGAACAGCAACAAGCTGGACAGCAA | |
| | GGTGGGCGGCAACTACAACTACCTGTACAGGCTGTTCAGGAAGAGCAACCT | |
| | GAAGCCCTTCGAGAGGGACATCAGCACCGAGATCTACCAGGCCGGCAACA | |
| | AGCCCTGCAACGGCGTGGCCGGCTTCAACTGCTACTTCCCCCTGAGGAGC | |
| | TACGGCTTCAGGCCCACCTACGGCGTGGGCCACCAGCCCTACAGGGTGGT | |
| | GGTGCTGAGCTTCGAGCTGCTGCACGCCCCCGCCACCGTGTGCGGCCCCA | |
| | AGAAGAGCACCAACCTGGTGAAGAACAAGTGCGTGAACTTCAACTTCAACG | |
| | GCCTGACCGGCACCGGCGTGCTGACCGAGAGCAACAAGAAGTTCCTGCCC | |
| | TTCCAGCAGTTCGGCAGGGACATCGCCGACACCACCGACGCCGTGAGGGA | |
| | CCCCCAGACCCTGGAGATCCTGGACATCACCCCCTGCAGCTTCGGCGGCG | |
| | TGAGCGTGATCACCCCCGGCACCAACACCAGCAACCAGGTGGCCGTGCTG | |
| | TACCAGGGCGTGAACTGCACCGAGGTGCCCGTGGCCATCCACGCCGACCA | |
| | GCTGACCCCCACCTGGAGGGTGTACAGCACCGGCAGCAACGTGTTCCAGA | |
| | CCAGGGCCGGCTGCCTGATCGGCGCCGAGTACGTGAACAACAGCTACGAG | |
| | TGCGACATCCCCATCGGCGCCGGCATCTGCGCCAGCTACCAGACCCAGAC | |
| | CAAGAGCCACCAGCAGGCCCAGAGCGTGGCCAGCCAGAGCATCATCGCCT | |
| | ACACCATGAGCCTGGGCGCCGAGAACAGCGTGGCCTACAGCAACAACAGC | |
| | ATCGCCATCCCCACCAACTTCACCATCAGCGTGACCACCGAGATCCTGCCC | |
| | GTGAGCATGACCAAGACCAGCGTGGACTGCACCATGTACATCTGCGGCGA | |
| | CAGCACCGAGTGCAGCAACCTGCTGCTGCAGTACGGCAGCTTCTGCACCC | |
| | AGCTGAAGAGGGCCCTGACCGGCATCGCCGTGGAGCAGGACAAGAACACC | |
| | CAGGAGGTGTTCGCCCAGGTGAAGCAGATCTACAAGACCCCCCCCATCAA | |
| | GTACTTCGGCGGCTTCAACTTCAGCCAGATCCTGCCCGACCCCAGCAAGCC | |
| | CAGCAAGAGGAGCTTCATCGAGGACCTGCTGTTCAACAAGGTGACCCTGGC | |
| | CGACGCCGGCTTCATCAAGCAGTACGGCGACTGCCTGGGCGACATCGCCG | |
| | CCAGGGACCTGATCTGCGCCCAGAAGTTCAACGGCCTGACCGTGCTGCCC | |
| | CCCCTGCTGACCGACGAGATGATCGCCCAGTACACCAGCGCCCTGCTGGC | |
| | CGGCACCATCACCAGCGGCTGGACCTTCGGCGCCGGCGCCGCCCTGCAG | |
| | ATCCCCTTCGCCATGCAGATGGCCTACAGGTTCAACGGCATCGGCGTGACC | |
| | CAGAACGTGCTGTACGAGAACCAGAAGCTGATCGCCAACCAGTTCAACAGC | |
| | GCCATCGGCAAGATCCAGGACAGCCTGAGCAGCACCGCCAGCGCCCTGGG | |
| | CAAGCTGCAGGACGTGGTGAACCACAACGCCCAGGCCCTGAACACCCTGG | |
| | TGAAGCAGCTGAGCAGCAAGTTCGGCGCCATCAGCAGCGTGCTGAACGAC | |
| | ATCCTGAGCAGGCTGGACCCCCCCGAGGCCGAGGTGCAGATCGACAGGCT | |
| | GATCACCGGCAGGCTGCAGAGCCTGCAGACCTACGTGACCCAGCAGCTGA | |
| | TCAGGGCCGCCGAGATCAGGGCCAGCGCCAACCTGGCCGCCACCAAGATG | |
| | AGCGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTGTTC | |
| | CTGCACGTGACCTACGTGCCCGCCCAGGAGAAGAACTTCACCACCGCCCC | |
| | CGCCATCTGCCACGACGGCAAGGCCCACTTCCCCAGGGAGGGCGTGTTCG | |
| | TGAGCAACGGCACCCACTGGTTCGTGACCCAGAGGAACTTCTACGAGCCC | |
| | CAGATCATCACCACCGACAACACCTTCGTGAGCGGCAACTGCGACGTGGTG | |
| | ATCGGCATCGTGAACAACACCGTGTACGACCCCCTGCAGCCCGAGCTGGA | |
| | CAGCTTCAAGGAGGAGCTGGACAAGTACTTCAAGAACCACACCAGCCCCGA | |
| | CGTGGACCTGGGCGACATCAGCGGCATCAACGCCAGCGTGGTGAACATCC | |
| | AGAAGGAGATCGACAGGCTGAACGAGGTGGCCAAGAACCTGAACGAGAGC | |
| | CTGATCGACCTGCAGGAGCTGGGCAAGTACGAGCAGTACATCAAGTGGCC | |
| | CTGGTACATCTGGCTGGGCTTCATCGCCGGCCTGATCGCCATCGTGATGGT | |
| | GACCATCATGCTGTGCTGCATGACCAGCTGCTGCAGCTGCCTGAAGGGCT | |
| | GCTGCAGCTGCGGCAGCTGCTGCAAGTTCGACGAGGACGACAGCGAGCCC | |
| | GTGCTGAAGGGCGTGAAGCTGCACTACACCGGAAGCGGAGCCACGAACTT | |
| | CTCTCTGTTAAAGCAAGCAGGAGATGTTGAAGAAAACCCCGGGCCTATGAA | |
| | GGCAATACTAGTAGTTCTGCTATATACATTTGCAACCGCAAATGCAGACACA | |
| | TTATGTATAGGTTATCATGCGAACAACTCAACAGACACTGTAGACACAGTAC | |
| | TAGAAAAGAATGTAACAGTAACACACTCTGTTAACCTTCTAGAAGACAAGCA | |
| | TAACGGGAAACTATGCAAACTAAGAGGGGTAGCCCCATTGCATTTGGGTAA | |
| | ATGTAACATTGCTGGCTGGATCCTGGGAAATCCAGAGTGTGAATCACTCTC | |
| | CACAGCAAGCTCATGGTCCTACATTGTGGAAACATCTAGTTCAGACAATGGA | |
| | ACGTGTTACCCAGGAGATTTCATCGATTATGAGGAGCTAAGAGAGCAATTGA | |
| | GCTCAGTGTCATCATTTGAAAGGTTTGAGATATTCCCCAAGACAAGTTCATG | |
| | GCCCAATCATGACTCGAACAAAGGTGTAACGACAGCATGTCCTCATGCTGG | |
| | AGCAAAAAGCTTCTACAAAAATTTAATATGGCTAGTTAAAAAAGGAAATTCAT | |
| | ACCCAAAGCTCAGCAAATCCTACATTAATGATAAAGGGAAAGAAGTCCTCGT | |
| | GCTATGGGGCATTCACCATCCACCTACTAGTGCTGACCAACAAAGTCTCTAT | |
| | CAGAATGCAGATGCATATGTTTTTGTGGGGACATCAAGATACAGCAAGAAGT | |
| | TCAAGCCGGAAATAGCAATAAGGCCCAAAGTGAGGGATCAAGAAGGGAGAA | |
| | TGAACTATTACTGGACACTAGTAGAGCCGGGAGACAAAATAACATTCGAAG | |
| | CAACTGGAAATCTAGTGGTACCGAGATATGCATTCGCAATGGAAAGAAATG | |
| | CTGGATCTGGTATTATCATTTCAGATACACCAGTCCACGATTGCAATACAAC | |
| | TTGTCAGACACCCAAGGGTGCTATAAACACCAGCCTCCCATTTCAGAACATA | |
| | CATCCGATCACAATTGGAAAATGTCCAAAATATGTAAAAAGCACAAAATTGA | |
| | GACTGGCCACAGGATTGAGGAATGTCCCGTCCATTCAATCTAGAGGCCTAT | |
| | TTGGGGCCATTGCCGGTTTCATTGAAGGGGGGTGGACAGGGATGGTAGAT | |
| | GGATGGTACGGTTATCACCATCAAAATGAGCAGGGGTCAGGATATGCAGCC | |
| | GACCTGAAGAGCACACAGAATGCCATTGACGAGATTACTAACAAAGTAAACT | |
| | CTGTTATTGAAAAAATGAATACACAGTTCACAGCAGTAGGTAAAGAGTTCAA | |
| | CCACCTGGAAAAAAGAATAGAGAATTTAAATAAAAAAGTTGATGATGGTTTC | |
| | CTGGACATTTGGACTTACAATGCCGAACTGTTGGTTCTATTGGAAAATGAAA | |
| | GAACTTTGGACTACCACGATTCAAAGGTGAAGAACTTATATGAAAAGGTAAG | |
| | AAGCCAGTTAAAAAACAATGCCAAGGAAATTGGAAACGGCTGCTTTGAATTT | |
| | TACCACAAATGTGATAACACGTGCATGGAAAGTGTCAAAAATGGGACTTATG | |
| | ACTACCCAAAATACTCAGAGGAAGCAAAATTAAACAGAGAAGAAATAGATGG | |
| | GGTAAAGCTGGAATCAACAAGGATTTACCAGATTTTGGCGATCTATTCAACC | |
| | GTCGCCAGTTCATTGGTACTGGTAGTCTCCCTGGGGGCAATCAGTTTCTGG | |
| | ATGTGCTCTAATGGGTCTCTACAGTGTAGAATATGTATTTAAGGAAGCGGAG | |
| | CCACGAACTTCTCTCTGTTAAAGCAAGCAGGAGATGTTGAAGAAAACCCCG | |
| | GGCCTAGCAAAAGCAGGGGATAATTCTATTAACCATGAAGACTATCATTGCT | |
| | TTGAGCTACATTCTATGTCTGGTTTTCGCTCAAAAACTTCCTGGAAATGACAA | |
| | TAGCACTGCAACGCTGTGCCTTGGGCACCATGCAGTACCAAACGGAACGAT | |
| | AGTGAAAACAATCACGAATGACCAAATTGAAGTTACTAATGCTACTGAGCTG | |
| | GTTCAGAATTCCTCAATAGGTGAAATATGCGACAGTCCTCATCAGATCCTTG | |
| | ATGGAGAAAACTGCACACTAATAGATGCTCTATTGGGAGACCCTCAGTGTG | |
| | ATGGCTTTCAAAATAAGAAATGGGACCTTTTTGTTGAACGAAGCAAAGCCTA | |
| | CAGCAACTGTTACCCTTATGATGTGCCGGATTATGCCTCCCTTAGGTCACTA | |
| | GTTGCCTCATCCGGCACACTGGAGTTTAACAATGAAAGCTTCAATTGGGCT | |
| | GGAGTCACTCAAAACGGAACAAGTTCTGCTTGCATAAGGGGATCTAATAGTA | |
| | GTTTCTTTAGTAGATTAAATTGGTTGACCCACTTAAACTTCAAGTACCCAGCA | |
| | TTGAACGTGACTATGCCAAACAATGAACAATTTGACAAATTGTACATTTGGG | |
| | GGGTTCACCACCCGGGTACGGACAAGGACCAAATCTTCCTGTATGCTCAAT | |
| | CATCAGGAAGAATCACAGTATCTACCAAAAGAAGCCAACAAGCTGTAATCCC | |
| | GAATATCGGATCTAGACCCAGAATAAGGAATATCCCTAGCAGAATAAGCATC | |
| | TATTGGACAATAGTAAAACCGGGAGACATACTTTTGATTAACAGCACAGGGA | |
| | ATCTAATTGCTCCTAGGGGTTACTTCAAAATACGAAGTGGGAAAAGCTCAAT | |
| | AATGAGATCAGATGCACCCATTGGCAAATGCAAGTCTGAATGCATCACTCCA | |
| | AATGGAAGCATTCCCAATGACAAACCATTCCAAAATGTAAACAGGATCACAT | |
| | ACGGGGCCTGTCCCAGATATGTTAAGCAAAGCACTCTGAAATTGGCAACAG | |
| | GAATGCGAAATGTACCAGAGAAACAAACTAGAGGCATATTTGGCGCAATAG | |
| | CGGGTTTCATAGAAAATGGTTGGGAGGGAATGGTGGATGGTTGGTACGGTT | |
| | TCAGGCATCAAAATTCTGAGGGAAGAGGACAAGCAGCAGATCTCAAAAGCA | |
| | CTCAAGCAGCAATCGATCAAATCAATGGGAAGCTGAATCGATTGATCGGGA | |
| | AAACCAACGAGAAATTCCATCAGATTGAGAAAGAATTCTCAGAAGTAGAAGG | |
| | GAGAATTCAGGACCTTGAGAAATATGTTGAGGACACAAAAATAGATCTCTGG | |
| | TCATACAACGCAGAGCTTCTTGTTGCCCTGGAAAACCAACATACAATTGATC | |
| | TAACTGACTCAGAAATGAACAAACTGTTTGAAAAAACAAAGAAGCAACTGAG | |
| | GGAAAATGCTGAGGATATGGGCAATGGTTGTTTCAAAATATACCACAAATGT | |
| | GACAATGCCTGCATAGGATCAATCAGAAATGGAACTTATGACCACGATGTAT | |
| | ACAGGGATGAAGCATTAAACAACCGGTTCCAGATCAAGGGAGTTGAGCTGA | |
| | AGTCAGGGTACAAAGATTGGATCCTATGGATTTCCTTTGCCATATCATGTTTT | |
| | TTGCTTTGTGTTGCTTTGTTGGGGTTCATCATGTGGGCCTGCCAAAAGGGC | |
| | AACATTAGGTGCAACATTTGCATTTGAGTGCATTAATTAAAAACACCCTTGTT | |
| | TCTACTGGAAGCGGAGCCACGAACTTCTCTCTGTTAAAGCAAGCAGGAGAT | |
| | GTTGAAGAAAACCCCGGGCCTATTTTCTAATATCCACAAAATGAAGGCAATA | |
| | ATTGTACTACTCATGGTAGTAACATCCAATGCAGATCGAATCTGCACTGGGA | |
| | TAACATCGTCAAACTCACCACATGTCGTCAAAACTGCTACTCAAGGGGAGGT | |
| | CAACGTGACCGGTGTAATACCACTGACAACAACACCCACCAAATCTCATTTT | |
| | GCAAATCTCAAAGGAACAGAAACCAGGGGGAAACTATGCCCAAAATGCCTC | |
| | AACTGCACAGATCTGGATGTAGCCTTGGGCAGACCAAAATGCACAGGGAAA | |
| | ATACCCTCTGCAAGGGTTTCAATACTCCATGAAGTCAGACCTGTTACATCTG | |
| | GGTGCTTTCCTATAATGCACGATAGAACAAAAATTAGACAGCTGCCTAACCT | |
| | TCTCCGAGGATACGAACATGTCAGGTTATCAACTCACAACGTTATCAATGCA | |
| | AAAGATGCACCAGGAAGACCCTACGAAATTGGAACCTCAGGGTCTTGCCCT | |
| | AACATTACCAATGGAAACGGATTCTTCGCAACAATGGCTTGGGCCGTCCCA | |
| | AAAAACAAAACAGCAACAAATCCATTAACAATAGAAGTACCATACATTTGTAC | |
| | AGAAGGAGAAGACCAAATTACCGTTTGGGGGTTCCACTCTGACAACGAGAC | |
| | CCAAATGGCAAAGCTCTATGGGGACTCAAAGCCCCAGAAGTTCACCTCATC | |
| | TGCCAACGGAGTGACCACACATTACGTTTCACAGATTGGTGGCTTCCCAAAT | |
| | CAAACAGAAGACGGAGGACTACCACAAAGTGGCAGAATTGTTGTTGATTAC | |
| | ATGGTGCAGAAATCTGGAAAAACAGGAACAATTACCTATCAAAGAGGTATTT | |
| | TATTGCCTCAAAAAGTGTGGTGCGCAAGTGGCAGGAGCAAGGTAATAAAAG | |
| | GATCCTTGCCCTTAATTGGAGAAGCAGATTGCCTCCATGAAAAATACGGTG | |
| | GATTAAACAAAAGCAAGCCTTACTACACAGGGGAACATGCAAAGGCCATAG | |
| | GAAATTGCCCAATATGGGTGAAAACACCCTTGAAGCTGGCCAATGGAACCA | |
| | AATATAGACCCCCTGCAAAACTATTAAAGGAAAGAGGTTTCTTCGGAGCCAT | |
| | TGCTGGTTTCTTAGAGGGAGGATGGGAAGGAATGATTGCAGGTTGGCACG | |
| | GATACACATCCCATGGGGCACATGGAGTAGCGGTGGCAGCTGACCTTAAGA | |
| | GCACTCAAGAGGCCATAAACAAGATAACAAAAAATCTCAACTCTTTGAGTGA | |
| | GCTGGAAGTAAAGAATCTTCAAAGACTAAGCGGTGCCATGGATGAACTCCA | |
| | CAACGAAATACTAGAACTAGATGAGAAAGTGGATGATCTCAGAGCTGACAC | |
| | AATAAGCTCACAAATAGAACTCGCAGTCCTGCTTTCCAATGAAGGAATAATA | |
| | AACAGTGAAGATGAACATCTCTTGGCGCTTGAAAGAAAGCTGAAGAAAATG | |
| | CTGGGCCCCTCTGCTGTAGAGATAGGGAATGGATGCTTTGAAACCAAACAC | |
| | AAGTGCAACCAGACCTGCCTCGACAGAATAGCTGCTGGTACCTTTGATGCA | |
| | GGAGAATTTTCTCTCCCCACCTTTGATTCACTGAATATTACTGCTGCATCTTT | |
| | AAATGACGACGGATTGGACAATCATACTATACTGCTTTACTACTCAACTGCT | |
| | GCCTCCAGTTTGGCTGTAACACTGATGATAGCTATCTTTGTTGTTTATATGGT | |
| | CTCCAGAGACAATGTTTCTTGCTCCATTTGTCTATAAGGAAAGTTAAGCCCT | |
| | GTATTTTCCTTTATTGTAGTGCTTGTTTGCTTGTTGTCATTACAAAGAAACGT | |
| | TATTGAAAAATTGATGACTCGAGCTGGTACTGCATGCACGCAATGCTAGCTG | |
| | CCCCTTTCCCGTCCTGGGTACCCCGAGTCTCCCCCGACCTCGGGTCCCAG | |
| | GTATGCTCCCACCTCCACCTGCCCCACTCACCACCTCTGCTAGTTCCAGAC | |
| | ACCTCCCAAGCACGCAGCAATGCAGCTCAAAACGCTTAGCCTAGCCACACC | |
| | CCCACGGGAAACAGCAGTGATTAACCTTTAGCAATAAACGAAAGTTTAACTA | |
| | AGCTATACTAACCCCAGGGTTGGTCAATTTCGTGCCAGCCACACCCTGGAG | |
| | CTAGCAAAAAAAA | |
| Pan-CoV- | TAGTTATTAATAGTAATCAATTACGGGGTCATTAGTTCATAGCCCATATATGG | 39 |
| Flu | AGTTCCGCGTTACATAACTTACGGTAAATGGCCCGCCTGGCTGACCGCCCA | |
| Vaccine | ACGACCCCCGCCCATTGACGTCAATAATGACGTATGTTCCCATAGTAACGC | |
| #19 CoV- | CAATAGGGACTTTCCATTGACGTCAATGGGTGGAGTATTTACGGTAAACTGC | |
| Wuhan | CCACTTGGCAGTACATCAAGTGTATCATATGCCAAGTACGCCCCCTATTGAC | |
| Spike | GTCAATGACGGTAAATGGCCCGCCTGGCATTATGCCCAGTACATGACCTTA | |
| (HexaPro)- | TGGGACTTTCCTACTTGGCAGTACATCTACGTATTAGTCATCGCTATTACCA | |
| Influenza | TGGTGATGCGGTTTTGGCAGTACATCAATGGGCGTGGATAGCGGTTTGACT | |
| (HA) | CACGGGGATTTCCAAGTCTCCACCCCATTGACGTCAATGGGAGTTTGTTTTG | |
| | GCACCAAAATCAACGGGACTTTCCAAAATGTCGTAACAACTCCGCCCCATTG | |
| | ACGCAAATGGGCGGTAGGCGTGTACGGTGGGAGGTCTATATAAGCAGAGC | |
| | TGGTTTAGTGAACCGTCAGATCGAGAATAAACTAGTATTCTTCTGGTCCCCA | |
| | CAGACTCAGAGAGAACCCGCCACCATGTTCGTGTTCCTGGTGCTGCTGCCC | |
| | CTGGTGAGCAGCCAGTGCGTGAACCTGACCACCAGGACCCAGCTGCCCCC | |
| | CGCCTACACCAACAGCTTCACCAGGGGCGTGTACTACCCCGACAAGGTGTT | |
| | CAGGAGCAGCGTGCTGCACAGCACCCAGGACCTGTTCCTGCCCTTCTTCAG | |
| | CAACGTGACCTGGTTCCACGCCATCCACGTGAGCGGCACCAACGGCACCA | |
| | AGAGGTTCGACAACCCCGTGCTGCCCTTCAACGACGGCGTGTACTTCGCCA | |
| | GCACCGAGAAGAGCAACATCATCAGGGGCTGGATCTTCGGCACCACCCTG | |
| | GACAGCAAGACCCAGAGCCTGCTGATCGTGAACAACGCCACCAACGTGGT | |
| | GATCAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTACAG | |
| | CAGCGCCAACAACTGCACCTTCGAGTACGTGAGCCAGCCCTTCCTGATGGA | |
| | CCTGGAGGGCAAGCAGGGCAACTTCAAGAACCTGAGGGAGTTCGTGTTCA | |
| | AGAACATCGACGGCTACTTCAAGATCTACAGCAAGCACACCCCCATCAACC | |
| | TGGTGAGGGACCTGCCCCAGGGCTTCAGCGCCCTGGAGCCCCTGGTGGAC | |
| | CTGCCCATCGGCATCAACATCACCAGGTTCCAGACCCTGCTGGCCCTGCAC | |
| | AGGAGCTACCTGACCCCCGGCGACAGCAGCAGCGGCTGGACCGCCGGCG | |
| | CCGCCGCCTACTACGTGGGCTACCTGCAGCCCAGGACCTTCCTGCTGAAG | |
| | TACAACGAGAACGGCACCATCACCGACGCCGTGGACTGCGCCCTGGACCC | |
| | CCTGAGCGAGACCAAGTGCACCCTGAAGAGCTTCACCGTGGAGAAGGGCA | |
| | TCTACCAGACCAGCAACTTCAGGGTGCAGCCCACCGAGAGCATCGTGAGG | |
| | TTCCCCAACATCACCAACCTGTGCCCCTTCGGCGAGGTGTTCAACGCCACC | |
| | AGGTTCGCCAGCGTGTACGCCTGGAACAGGAAGAGGATCAGCAACTGCGT | |
| | GGCCGACTACAGCGTGCTGTACAACAGCGCCAGCTTCAGCACCTTCAAGTG | |
| | CTACGGCGTGAGCCCCACCAAGCTGAACGACCTGTGCTTCACCAACGTGTA | |
| | CGCCGACAGCTTCGTGATCAGGGGCGACGAGGTGAGGCAGATCGCCCCC | |
| | GGCCAGACCGGCAAGATCGCCGACTACAACTACAAGCTGCCCGACGACTT | |
| | CACCGGCTGCGTGATCGCCTGGAACAGCAACAACCTGGACAGCAAGGTGG | |
| | GCGGCAACTACAACTACCTGTACAGGCTGTTCAGGAAGAGCAACCTGAAGC | |
| | CCTTCGAGAGGGACATCAGCACCGAGATCTACCAGGCCGGCAGCACCCCC | |
| | TGCAACGGCGTGGAGGGCTTCAACTGCTACTTCCCCCTGCAGAGCTACGG | |
| | CTTCCAGCCCACCAACGGCGTGGGCTACCAGCCCTACAGGGTGGTGGTGC | |
| | TGAGCTTCGAGCTGCTGCACGCCCCCGCCACCGTGTGCGGCCCCAAGAAG | |
| | AGCACCAACCTGGTGAAGAACAAGTGCGTGAACTTCAACTTCAACGGCCTG | |
| | ACCGGCACCGGCGTGCTGACCGAGAGCAACAAGAAGTTCCTGCCCTTCCA | |
| | GCAGTTCGGCAGGGACATCGCCGACACCACCGACGCCGTGAGGGACCCC | |
| | CAGACCCTGGAGATCCTGGACATCACCCCCTGCAGCTTCGGCGGCGTGAG | |
| | CGTGATCACCCCCGGCACCAACACCAGCAACCAGGTGGCCGTGCTGTACC | |
| | AGGACGTGAACTGCACCGAGGTGCCCGTGGCCATCCACGCCGACCAGCTG | |
| | ACCCCCACCTGGAGGGTGTACAGCACCGGCAGCAACGTGTTCCAGACCAG | |
| | GGCCGGCTGCCTGATCGGCGCCGAGCACGTGAACAACAGCTACGAGTGCG | |
| | ACATCCCCATCGGCGCCGGCATCTGCGCCAGCTACCAGACCCAGACCAAC | |
| | AGCCCCGGCAGCGCCAGCAGCGTGGCCAGCCAGAGCATCATCGCCTACAC | |
| | CATGAGCCTGGGCGCCGAGAACAGCGTGGCCTACAGCAACAACAGCATCG | |
| | CCATCCCCACCAACTTCACCATCAGCGTGACCACCGAGATCCTGCCCGTGA | |
| | GCATGACCAAGACCAGCGTGGACTGCACCATGTACATCTGCGGCGACAGC | |
| | ACCGAGTGCAGCAACCTGCTGCTGCAGTACGGCAGCTTCTGCACCCAGCT | |
| | GAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACT | |
| | TCGGCGGCTTCAACTTCAGCCAGATCCTGCCCGACCCCAGCAAGCCCAGC | |
| | AAGAGGAGCCCCATCGAGGACCTGCTGTTCAACAAGGTGACCCTGGCCGA | |
| | CGCCGGCTTCATCAAGCAGTACGGCGACTGCCTGGGCGACATCGCCGCCA | |
| | GGGACCTGATCTGCGCCCAGAAGTTCAACGGCCTGACCGTGCTGCCCCCC | |
| | CTGCTGACCGACGAGATGATCGCCCAGTACACCAGCGCCCTGCTGGCCGG | |
| | CACCATCACCAGCGGCTGGACCTTCGGCGCCGGCCCCGCCCTGCAGATCC | |
| | CCTTCCCCATGCAGATGGCCTACAGGTTCAACGGCATCGGCGTGACCCAGA | |
| | ACGTGCTGTACGAGAACCAGAAGCTGATCGCCAACCAGTTCAACAGCGCCA | |
| | TCGGCAAGATCCAGGACAGCCTGAGCAGCACCCCCAGCGCCCTGGGCAAG | |
| | CTGCAGGACGTGGTGAACCAGAACGCCCAGGCCCTGAACACCCTGGTGAA | |
| | GCAGCTGAGCAGCAACTTCGGCGCCATCAGCAGCGTGCTGAACGACATCC | |
| | TGAGCAGGCTGGACCCCCCCGAGGCCGAGGTGCAGATCGACAGGCTGATC | |
| | ACCGGCAGGCTGCAGAGCCTGCAGACCTACGTGACCCAGCAGCTGATCAG | |
| | GGCCGCCGAGATCAGGGCCAGCGCCAACCTGGCCGCCACCAAGATGAGC | |
| | GAGTGCGTGCTGGGCCAGAGCAAGAGGGTGGACTTCTGCGGCAAGGGCTA | |
| | CCACCTGATGAGCTTCCCCCAGAGCGCCCCCCACGGCGTGGTGTTCCTGC | |
| | ACGTGACCTACGTGCCCGCCCAGGAGAAGAACTTCACCACCGCCCCCGCC | |
| | ATCTGCCACGACGGCAAGGCCCACTTCCCCAGGGAGGGCGTGTTCGTGAG | |
| | CAACGGCACCCACTGGTTCGTGACCCAGAGGAACTTCTACGAGCCCCAGAT | |
| | CATCACCACCGACAACACCTTCGTGAGCGGCAACTGCGACGTGGTGATCG | |
| | GCATCGTGAACAACACCGTGTACGACCCCCTGCAGCCCGAGCTGGACAGC | |
| | TTCAAGGAGGAGCTGGACAAGTACTTCAAGAACCACACCAGCCCCGACGTG | |
| | GACCTGGGCGACATCAGCGGCATCAACGCCAGCGTGGTGAACATCCAGAA | |
| | GGAGATCGACAGGCTGAACGAGGTGGCCAAGAACCTGAACGAGAGCCTGA | |
| | TCGACCTGCAGGAGCTGGGCAAGTACGAGCAGTACATCAAGTGGCCCTGG | |
| | TACATCTGGCTGGGCTTCATCGCCGGCCTGATCGCCATCGTGATGGTGACC | |
| | ATCATGCTGTGCTGCATGACCAGCTGCTGCAGCTGCCTGAAGGGCTGCTGC | |
| | AGCTGCGGCAGCTGCTGCAAGTTCGACGAGGACGACAGCGAGCCCGTGCT | |
| | GAAGGGCGTGAAGCTGCACTACACCGGAAGCGGAGCCACGAACTTCTCTC | |
| | TGTTAAAGCAAGCAGGAGATGTTGAAGAAAACCCCGGGCCTATGAAGGCAA | |
| | TACTAGTAGTTCTGCTATATACATTTGCAACCGCAAATGCAGACACATTATGT | |
| | ATAGGTTATCATGCGAACAACTCAACAGACACTGTAGACACAGTACTAGAAA | |
| | AGAATGTAACAGTAACACACTCTGTTAACCTTCTAGAAGACAAGCATAACGG | |
| | GAAACTATGCAAACTAAGAGGGGTAGCCCCATTGCATTTGGGTAAATGTAAC | |
| | ATTGCTGGCTGGATCCTGGGAAATCCAGAGTGTGAATCACTCTCCACAGCA | |
| | AGCTCATGGTCCTACATTGTGGAAACATCTAGTTCAGACAATGGAACGTGTT | |
| | ACCCAGGAGATTTCATCGATTATGAGGAGCTAAGAGAGCAATTGAGCTCAG | |
| | TGTCATCATTTGAAAGGTTTGAGATATTCCCCAAGACAAGTTCATGGCCCAA | |
| | TCATGACTCGAACAAAGGTGTAACGACAGCATGTCCTCATGCTGGAGCAAA | |
| | AAGCTTCTACAAAAATTTAATATGGCTAGTTAAAAAAGGAAATTCATACCCAA | |
| | AGCTCAGCAAATCCTACATTAATGATAAAGGGAAAGAAGTCCTCGTGCTATG | |
| | GGGCATTCACCATCCACCTACTAGTGCTGACCAACAAAGTCTCTATCAGAAT | |
| | GCAGATGCATATGTTTTTGTGGGGACATCAAGATACAGCAAGAAGTTCAAGC | |
| | CGGAAATAGCAATAAGGCCCAAAGTGAGGGATCAAGAAGGGAGAATGAACT | |
| | ATTACTGGACACTAGTAGAGCCGGGAGACAAAATAACATTCGAAGCAACTG | |
| | GAAATCTAGTGGTACCGAGATATGCATTCGCAATGGAAAGAAATGCTGGAT | |
| | CTGGTATTATCATTTCAGATACACCAGTCCACGATTGCAATACAACTTGTCA | |
| | GACACCCAAGGGTGCTATAAACACCAGCCTCCCATTTCAGAACATACATCC | |
| | GATCACAATTGGAAAATGTCCAAAATATGTAAAAAGCACAAAATTGAGACTG | |
| | GCCACAGGATTGAGGAATGTCCCGTCCATTCAATCTAGAGGCCTATTTGGG | |
| | GCCATTGCCGGTTTCATTGAAGGGGGGTGGACAGGGATGGTAGATGGATG | |
| | GTACGGTTATCACCATCAAAATGAGCAGGGGTCAGGATATGCAGCCGACCT | |
| | GAAGAGCACACAGAATGCCATTGACGAGATTACTAACAAAGTAAACTCTGTT | |
| | ATTGAAAAAATGAATACACAGTTCACAGCAGTAGGTAAAGAGTTCAACCACC | |
| | TGGAAAAAAGAATAGAGAATTTAAATAAAAAAGTTGATGATGGTTTCCTGGA | |
| | CATTTGGACTTACAATGCCGAACTGTTGGTTCTATTGGAAAATGAAAGAACT | |
| | TTGGACTACCACGATTCAAAGGTGAAGAACTTATATGAAAAGGTAAGAAGCC | |
| | AGTTAAAAAACAATGCCAAGGAAATTGGAAACGGCTGCTTTGAATTTTACCA | |
| | CAAATGTGATAACACGTGCATGGAAAGTGTCAAAAATGGGACTTATGACTAC | |
| | CCAAAATACTCAGAGGAAGCAAAATTAAACAGAGAAGAAATAGATGGGGTAA | |
| | AGCTGGAATCAACAAGGATTTACCAGATTTTGGCGATCTATTCAACCGTCGC | |
| | CAGTTCATTGGTACTGGTAGTCTCCCTGGGGGCAATCAGTTTCTGGATGTG | |
| | CTCTAATGGGTCTCTACAGTGTAGAATATGTATTTAAGGAAGCGGAGCCACG | |
| | AACTTCTCTCTGTTAAAGCAAGCAGGAGATGTTGAAGAAAACCCCGGGCCT | |
| | AGCAAAAGCAGGGGATAATTCTATTAACCATGAAGACTATCATTGCTTTGAG | |
| | CTACATTCTATGTCTGGTTTTCGCTCAAAAACTTCCTGGAAATGACAATAGCA | |
| | CTGCAACGCTGTGCCTTGGGCACCATGCAGTACCAAACGGAACGATAGTGA | |
| | AAACAATCACGAATGACCAAATTGAAGTTACTAATGCTACTGAGCTGGTTCA | |
| | GAATTCCTCAATAGGTGAAATATGCGACAGTCCTCATCAGATCCTTGATGGA | |
| | GAAAACTGCACACTAATAGATGCTCTATTGGGAGACCCTCAGTGTGATGGC | |
| | TTTCAAAATAAGAAATGGGACCTTTTTGTTGAACGAAGCAAAGCCTACAGCA | |
| | ACTGTTACCCTTATGATGTGCCGGATTATGCCTCCCTTAGGTCACTAGTTGC | |
| | CTCATCCGGCACACTGGAGTTTAACAATGAAAGCTTCAATTGGGCTGGAGT | |
| | CACTCAAAACGGAACAAGTTCTGCTTGCATAAGGGGATCTAATAGTAGTTTC | |
| | TTTAGTAGATTAAATTGGTTGACCCACTTAAACTTCAAGTACCCAGCATTGAA | |
| | CGTGACTATGCCAAACAATGAACAATTTGACAAATTGTACATTTGGGGGGTT | |
| | CACCACCCGGGTACGGACAAGGACCAAATCTTCCTGTATGCTCAATCATCA | |
| | GGAAGAATCACAGTATCTACCAAAAGAAGCCAACAAGCTGTAATCCCGAATA | |
| | TCGGATCTAGACCCAGAATAAGGAATATCCCTAGCAGAATAAGCATCTATTG | |
| | GACAATAGTAAAACCGGGAGACATACTTTTGATTAACAGCACAGGGAATCTA | |
| | ATTGCTCCTAGGGGTTACTTCAAAATACGAAGTGGGAAAAGCTCAATAATGA | |
| | GATCAGATGCACCCATTGGCAAATGCAAGTCTGAATGCATCACTCCAAATG | |
| | GAAGCATTCCCAATGACAAACCATTCCAAAATGTAAACAGGATCACATACGG | |
| | GGCCTGTCCCAGATATGTTAAGCAAAGCACTCTGAAATTGGCAACAGGAAT | |
| | GCGAAATGTACCAGAGAAACAAACTAGAGGCATATTTGGCGCAATAGCGGG | |
| | TTTCATAGAAAATGGTTGGGAGGGAATGGTGGATGGTTGGTACGGTTTCAG | |
| | GCATCAAAATTCTGAGGGAAGAGGACAAGCAGCAGATCTCAAAAGCACTCA | |
| | AGCAGCAATCGATCAAATCAATGGGAAGCTGAATCGATTGATCGGGAAAAC | |
| | CAACGAGAAATTCCATCAGATTGAGAAAGAATTCTCAGAAGTAGAAGGGAG | |
| | AATTCAGGACCTTGAGAAATATGTTGAGGACACAAAAATAGATCTCTGGTCA | |
| | TACAACGCAGAGCTTCTTGTTGCCCTGGAAAACCAACATACAATTGATCTAA | |
| | CTGACTCAGAAATGAACAAACTGTTTGAAAAAACAAAGAAGCAACTGAGGGA | |
| | AAATGCTGAGGATATGGGCAATGGTTGTTTCAAAATATACCACAAATGTGAC | |
| | AATGCCTGCATAGGATCAATCAGAAATGGAACTTATGACCACGATGTATACA | |
| | GGGATGAAGCATTAAACAACCGGTTCCAGATCAAGGGAGTTGAGCTGAAGT | |
| | CAGGGTACAAAGATTGGATCCTATGGATTTCCTTTGCCATATCATGTTTTTTG | |
| | CTTTGTGTTGCTTTGTTGGGGTTCATCATGTGGGCCTGCCAAAAGGGCAAC | |
| | ATTAGGTGCAACATTTGCATTTGAGTGCATTAATTAAAAACACCCTTGTTTCT | |
| | ACTGGAAGCGGAGCCACGAACTTCTCTCTGTTAAAGCAAGCAGGAGATGTT | |
| | GAAGAAAACCCCGGGCCTATTTTCTAATATCCACAAAATGAAGGCAATAATT | |
| | GTACTACTCATGGTAGTAACATCCAATGCAGATCGAATCTGCACTGGGATAA | |
| | CATCGTCAAACTCACCACATGTCGTCAAAACTGCTACTCAAGGGGAGGTCA | |
| | ACGTGACCGGTGTAATACCACTGACAACAACACCCACCAAATCTCATTTTGC | |
| | AAATCTCAAAGGAACAGAAACCAGGGGGAAACTATGCCCAAAATGCCTCAA | |
| | CTGCACAGATCTGGATGTAGCCTTGGGCAGACCAAAATGCACAGGGAAAAT | |
| | ACCCTCTGCAAGGGTTTCAATACTCCATGAAGTCAGACCTGTTACATCTGGG | |
| | TGCTTTCCTATAATGCACGATAGAACAAAAATTAGACAGCTGCCTAACCTTC | |
| | TCCGAGGATACGAACATGTCAGGTTATCAACTCACAACGTTATCAATGCAAA | |
| | AGATGCACCAGGAAGACCCTACGAAATTGGAACCTCAGGGTCTTGCCCTAA | |
| | CATTACCAATGGAAACGGATTCTTCGCAACAATGGCTTGGGCCGTCCCAAA | |
| | AAACAAAACAGCAACAAATCCATTAACAATAGAAGTACCATACATTTGTACAG | |
| | AAGGAGAAGACCAAATTACCGTTTGGGGGTTCCACTCTGACAACGAGACCC | |
| | AAATGGCAAAGCTCTATGGGGACTCAAAGCCCCAGAAGTTCACCTCATCTG | |
| | CCAACGGAGTGACCACACATTACGTTTCACAGATTGGTGGCTTCCCAAATCA | |
| | AACAGAAGACGGAGGACTACCACAAAGTGGCAGAATTGTTGTTGATTACAT | |
| | GGTGCAGAAATCTGGAAAAACAGGAACAATTACCTATCAAAGAGGTATTTTA | |
| | TTGCCTCAAAAAGTGTGGTGCGCAAGTGGCAGGAGCAAGGTAATAAAAGGA | |
| | TCCTTGCCCTTAATTGGAGAAGCAGATTGCCTCCATGAAAAATACGGTGGAT | |
| | TAAACAAAAGCAAGCCTTACTACACAGGGGAACATGCAAAGGCCATAGGAA | |
| | ATTGCCCAATATGGGTGAAAACACCCTTGAAGCTGGCCAATGGAACCAAAT | |
| | ATAGACCCCCTGCAAAACTATTAAAGGAAAGAGGTTTCTTCGGAGCCATTGC | |
| | TGGTTTCTTAGAGGGAGGATGGGAAGGAATGATTGCAGGTTGGCACGGATA | |
| | CACATCCCATGGGGCACATGGAGTAGCGGTGGCAGCTGACCTTAAGAGCA | |
| | CTCAAGAGGCCATAAACAAGATAACAAAAAATCTCAACTCTTTGAGTGAGCT | |
| | GGAAGTAAAGAATCTTCAAAGACTAAGCGGTGCCATGGATGAACTCCACAA | |
| | CGAAATACTAGAACTAGATGAGAAAGTGGATGATCTCAGAGCTGACACAATA | |
| | AGCTCACAAATAGAACTCGCAGTCCTGCTTTCCAATGAAGGAATAATAAACA | |
| | GTGAAGATGAACATCTCTTGGCGCTTGAAAGAAAGCTGAAGAAAATGCTGG | |
| | GCCCCTCTGCTGTAGAGATAGGGAATGGATGCTTTGAAACCAAACACAAGT | |
| | GCAACCAGACCTGCCTCGACAGAATAGCTGCTGGTACCTTTGATGCAGGAG | |
| | AATTTTCTCTCCCCACCTTTGATTCACTGAATATTACTGCTGCATCTTTAAAT | |
| | GACGACGGATTGGACAATCATACTATACTGCTTTACTACTCAACTGCTGCCT | |
| | CCAGTTTGGCTGTAACACTGATGATAGCTATCTTTGTTGTTTATATGGTCTCC | |
| | AGAGACAATGTTTCTTGCTCCATTTGTCTATAAGGAAAGTTAAGCCCTGTATT | |
| | TTCCTTTATTGTAGTGCTTGTTTGCTTGTTGTCATTACAAAGAAACGTTATTG | |
| | AAAAATTGATGACTCGAGCTGGTACTGCATGCACGCAATGCTAGCTGCCCC | |
| | TTTCCCGTCCTGGGTACCCCGAGTCTCCCCCGACCTCGGGTCCCAGGTATG | |
| | CTCCCACCTCCACCTGCCCCACTCACCACCTCTGCTAGTTCCAGACACCTC | |
| | CCAAGCACGCAGCAATGCAGCTCAAAACGCTTAGCCTAGCCACACCCCCAC | |
| | GGGAAACAGCAGTGATTAACCTTTAGCAATAAACGAAAGTTTAACTAAGCTA | |
| | TACTAACCCCAGGGTTGGTCAATTTCGTGCCAGCCACACCCTGGAGCTAGC | |
| | AAAAAAAA | |
| Pan-CoV- | TAGTTATTAATAGTAATCAATTACGGGGTCATTAGTTCATAGCCCATATATGG | 40 |
| Flu | AGTTCCGCGTTACATAACTTACGGTAAATGGCCCGCCTGGCTGACCGCCCA | |
| Vaccine | ACGACCCCCGCCCATTGACGTCAATAATGACGTATGTTCCCATAGTAACGC | |
| #20 CoV- | CAATAGGGACTTTCCATTGACGTCAATGGGTGGAGTATTTACGGTAAACTGC | |
| Wuhan | CCACTTGGCAGTACATCAAGTGTATCATATGCCAAGTACGCCCCCTATTGAC | |
| Spike | GTCAATGACGGTAAATGGCCCGCCTGGCATTATGCCCAGTACATGACCTTA | |
| (2 proline | TGGGACTTTCCTACTTGGCAGTACATCTACGTATTAGTCATCGCTATTACCA | |
| subst.)- | TGGTGATGCGGTTTTGGCAGTACATCAATGGGCGTGGATAGCGGTTTGACT | |
| Influenza | CACGGGGATTTCCAAGTCTCCACCCCATTGACGTCAATGGGAGTTTGTTTTG | |
| (HA | GCACCAAAATCAACGGGACTTTCCAAAATGTCGTAACAACTCCGCCCCATTG | |
| | ACGCAAATGGGCGGTAGGCGTGTACGGTGGGAGGTCTATATAAGCAGAGC | |
| | TGGTTTAGTGAACCGTCAGATCGAGAATAAACTAGTATTCTTCTGGTCCCCA | |
| | CAGACTCAGAGAGAACCCGCCACCATGTTCGTGTTCCTGGTGCTGCTGCCC | |
| | CTGGTGAGCAGCCAGTGCGTGAACCTGACCACCAGGACCCAGCTGCCCCC | |
| | CGCCTACACCAACAGCTTCACCAGGGGCGTGTACTACCCCGACAAGGTGTT | |
| | CAGGAGCAGCGTGCTGCACAGCACCCAGGACCTGTTCCTGCCCTTCTTCAG | |
| | CAACGTGACCTGGTTCCACGCCATCCACGTGAGCGGCACCAACGGCACCA | |
| | AGAGGTTCGACAACCCCGTGCTGCCCTTCAACGACGGCGTGTACTTCGCCA | |
| | GCACCGAGAAGAGCAACATCATCAGGGGCTGGATCTTCGGCACCACCCTG | |
| | GACAGCAAGACCCAGAGCCTGCTGATCGTGAACAACGCCACCAACGTGGT | |
| | GATCAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTACAG | |
| | CAGCGCCAACAACTGCACCTTCGAGTACGTGAGCCAGCCCTTCCTGATGGA | |
| | CCTGGAGGGCAAGCAGGGCAACTTCAAGAACCTGAGGGAGTTCGTGTTCA | |
| | AGAACATCGACGGCTACTTCAAGATCTACAGCAAGCACACCCCCATCAACC | |
| | TGGTGAGGGACCTGCCCCAGGGCTTCAGCGCCCTGGAGCCCCTGGTGGAC | |
| | CTGCCCATCGGCATCAACATCACCAGGTTCCAGACCCTGCTGGCCCTGCAC | |
| | AGGAGCTACCTGACCCCCGGCGACAGCAGCAGCGGCTGGACCGCCGGCG | |
| | CCGCCGCCTACTACGTGGGCTACCTGCAGCCCAGGACCTTCCTGCTGAAG | |
| | TACAACGAGAACGGCACCATCACCGACGCCGTGGACTGCGCCCTGGACCC | |
| | CCTGAGCGAGACCAAGTGCACCCTGAAGAGCTTCACCGTGGAGAAGGGCA | |
| | TCTACCAGACCAGCAACTTCAGGGTGCAGCCCACCGAGAGCATCGTGAGG | |
| | TTCCCCAACATCACCAACCTGTGCCCCTTCGGCGAGGTGTTCAACGCCACC | |
| | AGGTTCGCCAGCGTGTACGCCTGGAACAGGAAGAGGATCAGCAACTGCGT | |
| | GGCCGACTACAGCGTGCTGTACAACAGCGCCAGCTTCAGCACCTTCAAGTG | |
| | CTACGGCGTGAGCCCCACCAAGCTGAACGACCTGTGCTTCACCAACGTGTA | |
| | CGCCGACAGCTTCGTGATCAGGGGCGACGAGGTGAGGCAGATCGCCCCC | |
| | GGCCAGACCGGCAAGATCGCCGACTACAACTACAAGCTGCCCGACGACTT | |
| | CACCGGCTGCGTGATCGCCTGGAACAGCAACAACCTGGACAGCAAGGTGG | |
| | GCGGCAACTACAACTACCTGTACAGGCTGTTCAGGAAGAGCAACCTGAAGC | |
| | CCTTCGAGAGGGACATCAGCACCGAGATCTACCAGGCCGGCAGCACCCCC | |
| | TGCAACGGCGTGGAGGGCTTCAACTGCTACTTCCCCCTGCAGAGCTACGG | |
| | CTTCCAGCCCACCAACGGCGTGGGCTACCAGCCCTACAGGGTGGTGGTGC | |
| | TGAGCTTCGAGCTGCTGCACGCCCCCGCCACCGTGTGCGGCCCCAAGAAG | |
| | AGCACCAACCTGGTGAAGAACAAGTGCGTGAACTTCAACTTCAACGGCCTG | |
| | ACCGGCACCGGCGTGCTGACCGAGAGCAACAAGAAGTTCCTGCCCTTCCA | |
| | GCAGTTCGGCAGGGACATCGCCGACACCACCGACGCCGTGAGGGACCCC | |
| | CAGACCCTGGAGATCCTGGACATCACCCCCTGCAGCTTCGGCGGCGTGAG | |
| | CGTGATCACCCCCGGCACCAACACCAGCAACCAGGTGGCCGTGCTGTACC | |
| | AGGACGTGAACTGCACCGAGGTGCCCGTGGCCATCCACGCCGACCAGCTG | |
| | ACCCCCACCTGGAGGGTGTACAGCACCGGCAGCAACGTGTTCCAGACCAG | |
| | GGCCGGCTGCCTGATCGGCGCCGAGCACGTGAACAACAGCTACGAGTGCG | |
| | ACATCCCCATCGGCGCCGGCATCTGCGCCAGCTACCAGACCCAGACCAAC | |
| | AGCCCCAGGAGGGCCAGGAGCGTGGCCAGCCAGAGCATCATCGCCTACAC | |
| | CATGAGCCTGGGCGCCGAGAACAGCGTGGCCTACAGCAACAACAGCATCG | |
| | CCATCCCCACCAACTTCACCATCAGCGTGACCACCGAGATCCTGCCCGTGA | |
| | GCATGACCAAGACCAGCGTGGACTGCACCATGTACATCTGCGGCGACAGC | |
| | ACCGAGTGCAGCAACCTGCTGCTGCAGTACGGCAGCTTCTGCACCCAGCT | |
| | GAACAGGGCCCTGACCGGCATCGCCGTGGAGCAGGACAAGAACACCCAGG | |
| | AGGTGTTCGCCCAGGTGAAGCAGATCTACAAGACCCCCCCCATCAAGGACT | |
| | TCGGCGGCTTCAACTTCAGCCAGATCCTGCCCGACCCCAGCAAGCCCAGC | |
| | AAGAGGAGCTTCATCGAGGACCTGCTGTTCAACAAGGTGACCCTGGCCGAC | |
| | GCCGGCTTCATCAAGCAGTACGGCGACTGCCTGGGCGACATCGCCGCCAG | |
| | GGACCTGATCTGCGCCCAGAAGTTCAACGGCCTGACCGTGCTGCCCCCCC | |
| | TGCTGACCGACGAGATGATCGCCCAGTACACCAGCGCCCTGCTGGCCGGC | |
| | ACCATCACCAGCGGCTGGACCTTCGGCGCCGGCGCCGCCCTGCAGATCCC | |
| | CTTCGCCATGCAGATGGCCTACAGGTTCAACGGCATCGGCGTGACCCAGAA | |
| | CGTGCTGTACGAGAACCAGAAGCTGATCGCCAACCAGTTCAACAGCGCCAT | |
| | CGGCAAGATCCAGGACAGCCTGAGCAGCACCGCCAGCGCCCTGGGCAAG | |
| | CTGCAGGACGTGGTGAACCAGAACGCCCAGGCCCTGAACACCCTGGTGAA | |
| | GCAGCTGAGCAGCAACTTCGGCGCCATCAGCAGCGTGCTGAACGACATCC | |
| | TGAGCAGGCTGGACCCCCCCGAGGCCGAGGTGCAGATCGACAGGCTGATC | |
| | ACCGGCAGGCTGCAGAGCCTGCAGACCTACGTGACCCAGCAGCTGATCAG | |
| | GGCCGCCGAGATCAGGGCCAGCGCCAACCTGGCCGCCACCAAGATGAGC | |
| | GAGTGCGTGCTGGGCCAGAGCAAGAGGGTGGACTTCTGCGGCAAGGGCTA | |
| | CCACCTGATGAGCTTCCCCCAGAGCGCCCCCCACGGCGTGGTGTTCCTGC | |
| | ACGTGACCTACGTGCCCGCCCAGGAGAAGAACTTCACCACCGCCCCCGCC | |
| | ATCTGCCACGACGGCAAGGCCCACTTCCCCAGGGAGGGCGTGTTCGTGAG | |
| | CAACGGCACCCACTGGTTCGTGACCCAGAGGAACTTCTACGAGCCCCAGAT | |
| | CATCACCACCGACAACACCTTCGTGAGCGGCAACTGCGACGTGGTGATCG | |
| | GCATCGTGAACAACACCGTGTACGACCCCCTGCAGCCCGAGCTGGACAGC | |
| | TTCAAGGAGGAGCTGGACAAGTACTTCAAGAACCACACCAGCCCCGACGTG | |
| | GACCTGGGCGACATCAGCGGCATCAACGCCAGCGTGGTGAACATCCAGAA | |
| | GGAGATCGACAGGCTGAACGAGGTGGCCAAGAACCTGAACGAGAGCCTGA | |
| | TCGACCTGCAGGAGCTGGGCAAGTACGAGCAGTACATCAAGTGGCCCTGG | |
| | TACATCTGGCTGGGCTTCATCGCCGGCCTGATCGCCATCGTGATGGTGACC | |
| | ATCATGCTGTGCTGCATGACCAGCTGCTGCAGCTGCCTGAAGGGCTGCTGC | |
| | AGCTGCGGCAGCTGCTGCAAGTTCGACGAGGACGACAGCGAGCCCGTGCT | |
| | GAAGGGCGTGAAGCTGCACTACACCGGAAGCGGAGCCACGAACTTCTCTC | |
| | TGTTAAAGCAAGCAGGAGATGTTGAAGAAAACCCCGGGCCTATGAAGGCAA | |
| | TACTAGTAGTTCTGCTATATACATTTGCAACCGCAAATGCAGACACATTATGT | |
| | ATAGGTTATCATGCGAACAACTCAACAGACACTGTAGACACAGTACTAGAAA | |
| | AGAATGTAACAGTAACACACTCTGTTAACCTTCTAGAAGACAAGCATAACGG | |
| | GAAACTATGCAAACTAAGAGGGGTAGCCCCATTGCATTTGGGTAAATGTAAC | |
| | ATTGCTGGCTGGATCCTGGGAAATCCAGAGTGTGAATCACTCTCCACAGCA | |
| | AGCTCATGGTCCTACATTGTGGAAACATCTAGTTCAGACAATGGAACGTGTT | |
| | ACCCAGGAGATTTCATCGATTATGAGGAGCTAAGAGAGCAATTGAGCTCAG | |
| | TGTCATCATTTGAAAGGTTTGAGATATTCCCCAAGACAAGTTCATGGCCCAA | |
| | TCATGACTCGAACAAAGGTGTAACGACAGCATGTCCTCATGCTGGAGCAAA | |
| | AAGCTTCTACAAAAATTTAATATGGCTAGTTAAAAAAGGAAATTCATACCCAA | |
| | AGCTCAGCAAATCCTACATTAATGATAAAGGGAAAGAAGTCCTCGTGCTATG | |
| | GGGCATTCACCATCCACCTACTAGTGCTGACCAACAAAGTCTCTATCAGAAT | |
| | GCAGATGCATATGTTTTTGTGGGGACATCAAGATACAGCAAGAAGTTCAAGC | |
| | CGGAAATAGCAATAAGGCCCAAAGTGAGGGATCAAGAAGGGAGAATGAACT | |
| | ATTACTGGACACTAGTAGAGCCGGGAGACAAAATAACATTCGAAGCAACTG | |
| | GAAATCTAGTGGTACCGAGATATGCATTCGCAATGGAAAGAAATGCTGGAT | |
| | CTGGTATTATCATTTCAGATACACCAGTCCACGATTGCAATACAACTTGTCA | |
| | GACACCCAAGGGTGCTATAAACACCAGCCTCCCATTTCAGAACATACATCC | |
| | GATCACAATTGGAAAATGTCCAAAATATGTAAAAAGCACAAAATTGAGACTG | |
| | GCCACAGGATTGAGGAATGTCCCGTCCATTCAATCTAGAGGCCTATTTGGG | |
| | GCCATTGCCGGTTTCATTGAAGGGGGGTGGACAGGGATGGTAGATGGATG | |
| | GTACGGTTATCACCATCAAAATGAGCAGGGGTCAGGATATGCAGCCGACCT | |
| | GAAGAGCACACAGAATGCCATTGACGAGATTACTAACAAAGTAAACTCTGTT | |
| | ATTGAAAAAATGAATACACAGTTCACAGCAGTAGGTAAAGAGTTCAACCACC | |
| | TGGAAAAAAGAATAGAGAATTTAAATAAAAAAGTTGATGATGGTTTCCTGGA | |
| | CATTTGGACTTACAATGCCGAACTGTTGGTTCTATTGGAAAATGAAAGAACT | |
| | TTGGACTACCACGATTCAAAGGTGAAGAACTTATATGAAAAGGTAAGAAGCC | |
| | AGTTAAAAAACAATGCCAAGGAAATTGGAAACGGCTGCTTTGAATTTTACCA | |
| | CAAATGTGATAACACGTGCATGGAAAGTGTCAAAAATGGGACTTATGACTAC | |
| | CCAAAATACTCAGAGGAAGCAAAATTAAACAGAGAAGAAATAGATGGGGTAA | |
| | AGCTGGAATCAACAAGGATTTACCAGATTTTGGCGATCTATTCAACCGTCGC | |
| | CAGTTCATTGGTACTGGTAGTCTCCCTGGGGGCAATCAGTTTCTGGATGTG | |
| | CTCTAATGGGTCTCTACAGTGTAGAATATGTATTTAAGGAAGCGGAGCCACG | |
| | AACTTCTCTCTGTTAAAGCAAGCAGGAGATGTTGAAGAAAACCCCGGGCCT | |
| | AGCAAAAGCAGGGGATAATTCTATTAACCATGAAGACTATCATTGCTTTGAG | |
| | CTACATTCTATGTCTGGTTTTCGCTCAAAAACTTCCTGGAAATGACAATAGCA | |
| | CTGCAACGCTGTGCCTTGGGCACCATGCAGTACCAAACGGAACGATAGTGA | |
| | AAACAATCACGAATGACCAAATTGAAGTTACTAATGCTACTGAGCTGGTTCA | |
| | GAATTCCTCAATAGGTGAAATATGCGACAGTCCTCATCAGATCCTTGATGGA | |
| | GAAAACTGCACACTAATAGATGCTCTATTGGGAGACCCTCAGTGTGATGGC | |
| | TTTCAAAATAAGAAATGGGACCTTTTTGTTGAACGAAGCAAAGCCTACAGCA | |
| | ACTGTTACCCTTATGATGTGCCGGATTATGCCTCCCTTAGGTCACTAGTTGC | |
| | CTCATCCGGCACACTGGAGTTTAACAATGAAAGCTTCAATTGGGCTGGAGT | |
| | CACTCAAAACGGAACAAGTTCTGCTTGCATAAGGGGATCTAATAGTAGTTTC | |
| | TTTAGTAGATTAAATTGGTTGACCCACTTAAACTTCAAGTACCCAGCATTGAA | |
| | CGTGACTATGCCAAACAATGAACAATTTGACAAATTGTACATTTGGGGGGTT | |
| | CACCACCCGGGTACGGACAAGGACCAAATCTTCCTGTATGCTCAATCATCA | |
| | GGAAGAATCACAGTATCTACCAAAAGAAGCCAACAAGCTGTAATCCCGAATA | |
| | TCGGATCTAGACCCAGAATAAGGAATATCCCTAGCAGAATAAGCATCTATTG | |
| | GACAATAGTAAAACCGGGAGACATACTTTTGATTAACAGCACAGGGAATCTA | |
| | ATTGCTCCTAGGGGTTACTTCAAAATACGAAGTGGGAAAAGCTCAATAATGA | |
| | GATCAGATGCACCCATTGGCAAATGCAAGTCTGAATGCATCACTCCAAATG | |
| | GAAGCATTCCCAATGACAAACCATTCCAAAATGTAAACAGGATCACATACGG | |
| | GGCCTGTCCCAGATATGTTAAGCAAAGCACTCTGAAATTGGCAACAGGAAT | |
| | GCGAAATGTACCAGAGAAACAAACTAGAGGCATATTTGGCGCAATAGCGGG | |
| | TTTCATAGAAAATGGTTGGGAGGGAATGGTGGATGGTTGGTACGGTTTCAG | |
| | GCATCAAAATTCTGAGGGAAGAGGACAAGCAGCAGATCTCAAAAGCACTCA | |
| | AGCAGCAATCGATCAAATCAATGGGAAGCTGAATCGATTGATCGGGAAAAC | |
| | CAACGAGAAATTCCATCAGATTGAGAAAGAATTCTCAGAAGTAGAAGGGAG | |
| | AATTCAGGACCTTGAGAAATATGTTGAGGACACAAAAATAGATCTCTGGTCA | |
| | TACAACGCAGAGCTTCTTGTTGCCCTGGAAAACCAACATACAATTGATCTAA | |
| | CTGACTCAGAAATGAACAAACTGTTTGAAAAAACAAAGAAGCAACTGAGGGA | |
| | AAATGCTGAGGATATGGGCAATGGTTGTTTCAAAATATACCACAAATGTGAC | |
| | AATGCCTGCATAGGATCAATCAGAAATGGAACTTATGACCACGATGTATACA | |
| | GGGATGAAGCATTAAACAACCGGTTCCAGATCAAGGGAGTTGAGCTGAAGT | |
| | CAGGGTACAAAGATTGGATCCTATGGATTTCCTTTGCCATATCATGTTTTTTG | |
| | CTTTGTGTTGCTTTGTTGGGGTTCATCATGTGGGCCTGCCAAAAGGGCAAC | |
| | ATTAGGTGCAACATTTGCATTTGAGTGCATTAATTAAAAACACCCTTGTTTCT | |
| | ACTGGAAGCGGAGCCACGAACTTCTCTCTGTTAAAGCAAGCAGGAGATGTT | |
| | GAAGAAAACCCCGGGCCTATTTTCTAATATCCACAAAATGAAGGCAATAATT | |
| | GTACTACTCATGGTAGTAACATCCAATGCAGATCGAATCTGCACTGGGATAA | |
| | CATCGTCAAACTCACCACATGTCGTCAAAACTGCTACTCAAGGGGAGGTCA | |
| | ACGTGACCGGTGTAATACCACTGACAACAACACCCACCAAATCTCATTTTGC | |
| | AAATCTCAAAGGAACAGAAACCAGGGGGAAACTATGCCCAAAATGCCTCAA | |
| | CTGCACAGATCTGGATGTAGCCTTGGGCAGACCAAAATGCACAGGGAAAAT | |
| | ACCCTCTGCAAGGGTTTCAATACTCCATGAAGTCAGACCTGTTACATCTGGG | |
| | TGCTTTCCTATAATGCACGATAGAACAAAAATTAGACAGCTGCCTAACCTTC | |
| | TCCGAGGATACGAACATGTCAGGTTATCAACTCACAACGTTATCAATGCAAA | |
| | AGATGCACCAGGAAGACCCTACGAAATTGGAACCTCAGGGTCTTGCCCTAA | |
| | CATTACCAATGGAAACGGATTCTTCGCAACAATGGCTTGGGCCGTCCCAAA | |
| | AAACAAAACAGCAACAAATCCATTAACAATAGAAGTACCATACATTTGTACAG | |
| | AAGGAGAAGACCAAATTACCGTTTGGGGGTTCCACTCTGACAACGAGACCC | |
| | AAATGGCAAAGCTCTATGGGGACTCAAAGCCCCAGAAGTTCACCTCATCTG | |
| | CCAACGGAGTGACCACACATTACGTTTCACAGATTGGTGGCTTCCCAAATCA | |
| | AACAGAAGACGGAGGACTACCACAAAGTGGCAGAATTGTTGTTGATTACAT | |
| | GGTGCAGAAATCTGGAAAAACAGGAACAATTACCTATCAAAGAGGTATTTTA | |
| | TTGCCTCAAAAAGTGTGGTGCGCAAGTGGCAGGAGCAAGGTAATAAAAGGA | |
| | TCCTTGCCCTTAATTGGAGAAGCAGATTGCCTCCATGAAAAATACGGTGGAT | |
| | TAAACAAAAGCAAGCCTTACTACACAGGGGAACATGCAAAGGCCATAGGAA | |
| | ATTGCCCAATATGGGTGAAAACACCCTTGAAGCTGGCCAATGGAACCAAAT | |
| | ATAGACCCCCTGCAAAACTATTAAAGGAAAGAGGTTTCTTCGGAGCCATTGC | |
| | TGGTTTCTTAGAGGGAGGATGGGAAGGAATGATTGCAGGTTGGCACGGATA | |
| | CACATCCCATGGGGCACATGGAGTAGCGGTGGCAGCTGACCTTAAGAGCA | |
| | CTCAAGAGGCCATAAACAAGATAACAAAAAATCTCAACTCTTTGAGTGAGCT | |
| | GGAAGTAAAGAATCTTCAAAGACTAAGCGGTGCCATGGATGAACTCCACAA | |
| | CGAAATACTAGAACTAGATGAGAAAGTGGATGATCTCAGAGCTGACACAATA | |
| | AGCTCACAAATAGAACTCGCAGTCCTGCTTTCCAATGAAGGAATAATAAACA | |
| | GTGAAGATGAACATCTCTTGGCGCTTGAAAGAAAGCTGAAGAAAATGCTGG | |
| | GCCCCTCTGCTGTAGAGATAGGGAATGGATGCTTTGAAACCAAACACAAGT | |
| | GCAACCAGACCTGCCTCGACAGAATAGCTGCTGGTACCTTTGATGCAGGAG | |
| | AATTTTCTCTCCCCACCTTTGATTCACTGAATATTACTGCTGCATCTTTAAAT | |
| | GACGACGGATTGGACAATCATACTATACTGCTTTACTACTCAACTGCTGCCT | |
| | CCAGTTTGGCTGTAACACTGATGATAGCTATCTTTGTTGTTTATATGGTCTCC | |
| | AGAGACAATGTTTCTTGCTCCATTTGTCTATAAGGAAAGTTAAGCCCTGTATT | |
| | TTCCTTTATTGTAGTGCTTGTTTGCTTGTTGTCATTACAAAGAAACGTTATTG | |
| | AAAAATTGATGACTCGAGCTGGTACTGCATGCACGCAATGCTAGCTGCCCC | |
| | TTTCCCGTCCTGGGTACCCCGAGTCTCCCCCGACCTCGGGTCCCAGGTATG | |
| | CTCCCACCTCCACCTGCCCCACTCACCACCTCTGCTAGTTCCAGACACCTC | |
| | CCAAGCACGCAGCAATGCAGCTCAAAACGCTTAGCCTAGCCACACCCCCAC | |
| | GGGAAACAGCAGTGATTAACCTTTAGCAATAAACGAAAGTTTAACTAAGCTA | |
| | TACTAACCCCAGGGTTGGTCAATTTCGTGCCAGCCACACCCTGGAGCTAGC | |
| | AAAAAAAA | |
| Pan-CoV- | CTCGACATTGATTATTGACTAGTTATTAATAGTAATCAATTACGGGGTCATTA | 41 |
| Flu | GTTCATAGCCCATATATGGAGTTCCGCGTTACATAACTTACGGTAAATGGCC | |
| Vaccine | CGCCTGGCTGACCGCCCAACGACCCCCGCCCATTGACGTCAATAATGACGT | |
| #21 CoV- | ATGTTCCCATAGTAACGCCAATAGGGACTTTCCATTGACGTCAATGGGTGGA | |
| Wuhan | GTATTTACGGTAAACTGCCCACTTGGCAGTACATCAAGTGTATCATATGCCA | |
| Spike | AGTACGCCCCCTATTGACGTCAATGACGGTAAATGGCCCGCCTGGCATTAT | |
| (HexaPro)- | GCCCAGTACATGACCTTATGGGACTTTCCTACTTGGCAGTACATCTACGTAT | |
| Influenza | TAGTCATCGCTATTACCATGGTCGAGGTGAGCCCCACGTTCTGCTTCACTCT | |
| (HA) | CCCCATCTCCCCCCCCTCCCCACCCCCAATTTTGTATTTATTTATTTTTTAAT | |
| | TATTTTGTGCAGCGATGGGGGCGGGGGGGGGGGGGGGGCGCGCGCCAG | |
| | GCGGGGCGGGGCGGGGCGAGGGGCGGGGCGGGGCGAGGCGGAGAGGT | |
| | GCGGCGGCAGCCAATCAGAGCGGCGCGCTCCGAAAGTTTCCTTTTATGGC | |
| | GAGGCGGCGGCGGCGGCGGCCCTATAAAAAGCGAAGCGCGCGGGGGGC | |
| | GGGAGTCGCTGCGCGCTGCCTTCGCCCCGTGCCCCGCTCCGCCGCCGCC | |
| | TCGCGCCGCCCGCCCCGGCTCTGACTGACCGCGTTACTCCCACAGGTGAG | |
| | CGGGCGGGACGGCCCTTCTCCTCCGGGCTGTAATTAGCGCTTGGTTTAATG | |
| | ACGGCTTGTTTCTTTTCTGTGGCTGCGTGAAAGCCTTGAGGGGCTCCGGGA | |
| | GGGCCCTTTGTGCGGGGGGAGCGGCTCGGGGGGTGCGTGCGTGTGTGTG | |
| | TGCGTGGGGAGCGCCGCGTGCGGCTCCGCGCTGCCCGGCGGCTGTGAGC | |
| | GCTGCGGGCGCGGCGCGGGGCTTTGTGCGCTCCGCAGTGTGCGCGAGGG | |
| | GAGCGCGGCCGGGGGGGGTGCCCCGCGGTGCGGGGGGGGCTGCGAGGG | |
| | GAACAAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCCCGAGT | |
| | TGCTGAGCACGGCCCGGCTTCGGGTGCGGGGCTCCGTACGGGGCGTGGC | |
| | GCGGGGCTCGCCGTGCCGGGCGGGGGGTGGCGGCAGGTGGGGGTGCCG | |
| | GGCGGGGCGGGGCCGCCTCGGGCCGGGGAGGGCTCGGGGGAGGGGCG | |
| | CGGCGGCCCCCGGAGCGCCGGCGGCTGTCGAGGCGCGGCGAGCCGCAG | |
| | CCATTGCCTTTTATGGTAATCGTGCGAGAGGGCGCAGGGACTTCCTTTGTC | |
| | CCAAATCTGTGCGGAGCCGAAATCTGGGAGGCGCCGCCGCACCCCCTCTA | |
| | GCGGGCGCGGGGCGAAGCGGTGCGGCGCCGGCAGGAAGGAAATGGGCG | |
| | GGGAGGGCCTTCGTGCGTCGCCGCGCCGCCGTCCCCTTCTCCCTCTCCAG | |
| | CCTCGGGGCTGTCCGCGGGGGGACGGCTGCCTTCGGGGGGGACGGGGC | |
| | AGGGGGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGTGCTG | |
| | GTTATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTTCCT | |
| | GGTGCTGCTGCCCCTGGTGAGCAGCCAGTGCGTGAACCTGACCACCAGGA | |
| | CCCAGCTGCCCCCCGCCTACACCAACAGCTTCACCAGGGGCGTGTACTAC | |
| | CCCGACAAGGTGTTCAGGAGCAGCGTGCTGCACAGCACCCAGGACCTGTT | |
| | CCTGCCCTTCTTCAGCAACGTGACCTGGTTCCACGCCATCCACGTGAGCGG | |
| | CACCAACGGCACCAAGAGGTTCGACAACCCCGTGCTGCCCTTCAACGACG | |
| | GCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAAC | |
| | GCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAG | |
| | TTCAGGGTGTACAGCAGCGCCAACAACTGCACCTTCGAGTACGTGAGCCAG | |
| | CCCTTCCTGATGGACCTGGAGGGCAAGCAGGGCAACTTCAAGAACCTGAG | |
| | GGAGTTCGTGTTCAAGAACATCGACGGCTACTTCAAGATCTACAGCAAGCA | |
| | CACCCCCATCAACCTGGTGAGGGACCTGCCCCAGGGCTTCAGCGCCCTGG | |
| | AGCCCCTGGTGGACCTGCCCATCGGCATCAACATCACCAGGTTCCAGACCC | |
| | TGCTGGCCCTGCACAGGAGCTACCTGACCCCCGGCGACAGCAGCAGCGGC | |
| | TGGACCGCCGGCGCCGCCGCCTACTACGTGGGCTACCTGCAGCCCAGGAC | |
| | CTTCCTGCTGAAGTACAACGAGAACGGCACCATCACCGACGCCGTGGACTG | |
| | CGCCCTGGACCCCCTGAGCGAGACCAAGTGCACCCTGAAGAGCTTCACCG | |
| | TGGAGAAGGGCATCTACCAGACCAGCAACTTCAGGGTGCAGCCCACCGAG | |
| | AGCATCGTGAGGTTCCCCAACATCACCAACCTGTGCCCCTTCGGCGAGGTG | |
| | TTCAACGCCACCAGGTTCGCCAGCGTGTACGCCTGGAACAGGAAGAGGAT | |
| | CAGCAACTGCGTGGCCGACTACAGCGTGCTGTACAACAGCGCCAGCTTCA | |
| | GCACCTTCAAGTGCTACGGCGTGAGCCCCACCAAGCTGAACGACCTGTGCT | |
| | TCACCAACGTGTACGCCGACAGCTTCGTGATCAGGGGCGACGAGGTGAGG | |
| | CAGATCGCCCCCGGCCAGACCGGCAAGATCGCCGACTACAACTACAAGCT | |
| | GCCCGACGACTTCACCGGCTGCGTGATCGCCTGGAACAGCAACAACCTGG | |
| | ACAGCAAGGTGGGGGGCAACTACAACTACCTGTACAGGCTGTTCAGGAAGA | |
| | GCAACCTGAAGCCCTTCGAGAGGGACATCAGCACCGAGATCTACCAGGCC | |
| | GGCAGCACCCCCTGCAACGGCGTGGAGGGCTTCAACTGCTACTTCCCCCT | |
| | GCAGAGCTACGGCTTCCAGCCCACCAACGGCGTGGGCTACCAGCCCTACA | |
| | GGGTGGTGGTGCTGAGCTTCGAGCTGCTGCACGCCCCCGCCACCGTGTGC | |
| | GGCCCCAAGAAGAGCACCAACCTGGTGAAGAACAAGTGCGTGAACTTCAAC | |
| | TTCAACGGCCTGACCGGCACCGGCGTGCTGACCGAGAGCAACAAGAAGTT | |
| | CCTGCCCTTCCAGCAGTTCGGCAGGGACATCGCCGACACCACCGACGCCG | |
| | TGAGGGACCCCCAGACCCTGGAGATCCTGGACATCACCCCCTGCAGCTTC | |
| | GGCGGCGTGAGCGTGATCACCCCCGGCACCAACACCAGCAACCAGGTGGC | |
| | CGTGCTGTACCAGGACGTGAACTGCACCGAGGTGCCCGTGGCCATCCACG | |
| | CCGACCAGCTGACCCCCACCTGGAGGGTGTACAGCACCGGCAGCAACGTG | |
| | TTCCAGACCAGGGCCGGCTGCCTGATCGGCGCCGAGCACGTGAACAACAG | |
| | CTACGAGTGCGACATCCCCATCGGCGCCGGCATCTGCGCCAGCTACCAGA | |
| | CCCAGACCAACAGCCCCGGCAGCGCCAGCAGCGTGGCCAGCCAGAGCAT | |
| | CATCGCCTACACCATGAGCCTGGGCGCCGAGAACAGCGTGGCCTACAGCA | |
| | ACAACAGCATCGCCATCCCCACCAACTTCACCATCAGCGTGACCACCGAGA | |
| | TCCTGCCCGTGAGCATGACCAAGACCAGCGTGGACTGCACCATGTACATCT | |
| | GCGGCGACAGCACCGAGTGCAGCAACCTGCTGCTGCAGTACGGCAGCTTC | |
| | TGCACCCAGCTGAACAGGGCCCTGACCGGCATCGCCGTGGAGCAGGACAA | |
| | GAACACCCAGGAGGTGTTCGCCCAGGTGAAGCAGATCTACAAGACCCCCC | |
| | CCATCAAGGACTTCGGCGGCTTCAACTTCAGCCAGATCCTGCCCGACCCCA | |
| | GCAAGCCCAGCAAGAGGAGCCCCATCGAGGACCTGCTGTTCAACAAGGTG | |
| | ACCCTGGCCGACGCCGGCTTCATCAAGCAGTACGGCGACTGCCTGGGCGA | |
| | CATCGCCGCCAGGGACCTGATCTGCGCCCAGAAGTTCAACGGCCTGACCG | |
| | TGCTGCCCCCCCTGCTGACCGACGAGATGATCGCCCAGTACACCAGCGCC | |
| | CTGCTGGCCGGCACCATCACCAGCGGCTGGACCTTCGGCGCCGGCCCCG | |
| | CCCTGCAGATCCCCTTCCCCATGCAGATGGCCTACAGGTTCAACGGCATCG | |
| | GCGTGACCCAGAACGTGCTGTACGAGAACCAGAAGCTGATCGCCAACCAG | |
| | TTCAACAGCGCCATCGGCAAGATCCAGGACAGCCTGAGCAGCACCCCCAG | |
| | CGCCCTGGGCAAGCTGCAGGACGTGGTGAACCAGAACGCCCAGGCCCTGA | |
| | ACACCCTGGTGAAGCAGCTGAGCAGCAACTTCGGCGCCATCAGCAGCGTG | |
| | CTGAACGACATCCTGAGCAGGCTGGACCCCCCCGAGGCCGAGGTGCAGAT | |
| | CGACAGGCTGATCACCGGCAGGCTGCAGAGCCTGCAGACCTACGTGACCC | |
| | AGCAGCTGATCAGGGCCGCCGAGATCAGGGCCAGCGCCAACCTGGCCGC | |
| | CACCAAGATGAGCGAGTGCGTGCTGGGCCAGAGCAAGAGGGTGGACTTCT | |
| | GCGGCAAGGGCTACCACCTGATGAGCTTCCCCCAGAGCGCCCCCCACGGC | |
| | GTGGTGTTCCTGCACGTGACCTACGTGCCCGCCCAGGAGAAGAACTTCACC | |
| | ACCGCCCCCGCCATCTGCCACGACGGCAAGGCCCACTTCCCCAGGGAGGG | |
| | CGTGTTCGTGAGCAACGGCACCCACTGGTTCGTGACCCAGAGGAACTTCTA | |
| | CGAGCCCCAGATCATCACCACCGACAACACCTTCGTGAGCGGCAACTGCG | |
| | ACGTGGTGATCGGCATCGTGAACAACACCGTGTACGACCCCCTGCAGCCC | |
| | GAGCTGGACAGCTTCAAGGAGGAGCTGGACAAGTACTTCAAGAACCACACC | |
| | AGCCCCGACGTGGACCTGGGCGACATCAGCGGCATCAACGCCAGCGTGGT | |
| | GAACATCCAGAAGGAGATCGACAGGCTGAACGAGGTGGCCAAGAACCTGA | |
| | ACGAGAGCCTGATCGACCTGCAGGAGCTGGGCAAGTACGAGCAGTACATC | |
| | AAGTGGCCCTGGTACATCTGGCTGGGCTTCATCGCCGGCCTGATCGCCATC | |
| | GTGATGGTGACCATCATGCTGTGCTGCATGACCAGCTGCTGCAGCTGCCTG | |
| | AAGGGCTGCTGCAGCTGCGGCAGCTGCTGCAAGTTCGACGAGGACGACAG | |
| | CGAGCCCGTGCTGAAGGGCGTGAAGCTGCACTACACCGGAAGCGGAGCCA | |
| | CGAACTTCTCTCTGTTAAAGCAAGCAGGAGATGTTGAAGAAAACCCCGGGC | |
| | CTATGAAGGCAATACTAGTAGTTCTGCTATATACATTTGCAACCGCAAATGC | |
| | AGACACATTATGTATAGGTTATCATGCGAACAACTCAACAGACACTGTAGAC | |
| | ACAGTACTAGAAAAGAATGTAACAGTAACACACTCTGTTAACCTTCTAGAAG | |
| | ACAAGCATAACGGGAAACTATGCAAACTAAGAGGGGTAGCCCCATTGCATT | |
| | TGGGTAAATGTAACATTGCTGGCTGGATCCTGGGAAATCCAGAGTGTGAAT | |
| | CACTCTCCACAGCAAGCTCATGGTCCTACATTGTGGAAACATCTAGTTCAGA | |
| | CAATGGAACGTGTTACCCAGGAGATTTCATCGATTATGAGGAGCTAAGAGA | |
| | GCAATTGAGCTCAGTGTCATCATTTGAAAGGTTTGAGATATTCCCCAAGACA | |
| | AGTTCATGGCCCAATCATGACTCGAACAAAGGTGTAACGACAGCATGTCCT | |
| | CATGCTGGAGCAAAAAGCTTCTACAAAAATTTAATATGGCTAGTTAAAAAAG | |
| | GAAATTCATACCCAAAGCTCAGCAAATCCTACATTAATGATAAAGGGAAAGA | |
| | AGTCCTCGTGCTATGGGGCATTCACCATCCACCTACTAGTGCTGACCAACA | |
| | AAGTCTCTATCAGAATGCAGATGCATATGTTTTTGTGGGGACATCAAGATAC | |
| | AGCAAGAAGTTCAAGCCGGAAATAGCAATAAGGCCCAAAGTGAGGGATCAA | |
| | GAAGGGAGAATGAACTATTACTGGACACTAGTAGAGCCGGGAGACAAAATA | |
| | ACATTCGAAGCAACTGGAAATCTAGTGGTACCGAGATATGCATTCGCAATG | |
| | GAAAGAAATGCTGGATCTGGTATTATCATTTCAGATACACCAGTCCACGATT | |
| | GCAATACAACTTGTCAGACACCCAAGGGTGCTATAAACACCAGCCTCCCATT | |
| | TCAGAACATACATCCGATCACAATTGGAAAATGTCCAAAATATGTAAAAAGC | |
| | ACAAAATTGAGACTGGCCACAGGATTGAGGAATGTCCCGTCCATTCAATCTA | |
| | GAGGCCTATTTGGGGCCATTGCCGGTTTCATTGAAGGGGGGTGGACAGGG | |
| | ATGGTAGATGGATGGTACGGTTATCACCATCAAAATGAGCAGGGGTCAGGA | |
| | TATGCAGCCGACCTGAAGAGCACACAGAATGCCATTGACGAGATTACTAAC | |
| | AAAGTAAACTCTGTTATTGAAAAAATGAATACACAGTTCACAGCAGTAGGTA | |
| | AAGAGTTCAACCACCTGGAAAAAAGAATAGAGAATTTAAATAAAAAAGTTGA | |
| | TGATGGTTTCCTGGACATTTGGACTTACAATGCCGAACTGTTGGTTCTATTG | |
| | GAAAATGAAAGAACTTTGGACTACCACGATTCAAAGGTGAAGAACTTATATG | |
| | AAAAGGTAAGAAGCCAGTTAAAAAACAATGCCAAGGAAATTGGAAACGGCT | |
| | GCTTTGAATTTTACCACAAATGTGATAACACGTGCATGGAAAGTGTCAAAAA | |
| | TGGGACTTATGACTACCCAAAATACTCAGAGGAAGCAAAATTAAACAGAGAA | |
| | GAAATAGATGGGGTAAAGCTGGAATCAACAAGGATTTACCAGATTTTGGCG | |
| | ATCTATTCAACCGTCGCCAGTTCATTGGTACTGGTAGTCTCCCTGGGGGCA | |
| | ATCAGTTTCTGGATGTGCTCTAATGGGTCTCTACAGTGTAGAATATGTATTTA | |
| | AGGAAGCGGAGCCACGAACTTCTCTCTGTTAAAGCAAGCAGGAGATGTTGA | |
| | AGAAAACCCCGGGCCTAGCAAAAGCAGGGGATAATTCTATTAACCATGAAG | |
| | ACTATCATTGCTTTGAGCTACATTCTATGTCTGGTTTTCGCTCAAAAACTTCC | |
| | TGGAAATGACAATAGCACTGCAACGCTGTGCCTTGGGCACCATGCAGTACC | |
| | AAACGGAACGATAGTGAAAACAATCACGAATGACCAAATTGAAGTTACTAAT | |
| | GCTACTGAGCTGGTTCAGAATTCCTCAATAGGTGAAATATGCGACAGTCCTC | |
| | ATCAGATCCTTGATGGAGAAAACTGCACACTAATAGATGCTCTATTGGGAGA | |
| | CCCTCAGTGTGATGGCTTTCAAAATAAGAAATGGGACCTTTTTGTTGAACGA | |
| | AGCAAAGCCTACAGCAACTGTTACCCTTATGATGTGCCGGATTATGCCTCCC | |
| | TTAGGTCACTAGTTGCCTCATCCGGCACACTGGAGTTTAACAATGAAAGCTT | |
| | CAATTGGGCTGGAGTCACTCAAAACGGAACAAGTTCTGCTTGCATAAGGGG | |
| | ATCTAATAGTAGTTTCTTTAGTAGATTAAATTGGTTGACCCACTTAAACTTCA | |
| | AGTACCCAGCATTGAACGTGACTATGCCAAACAATGAACAATTTGACAAATT | |
| | GTACATTTGGGGGGTTCACCACCCGGGTACGGACAAGGACCAAATCTTCCT | |
| | GTATGCTCAATCATCAGGAAGAATCACAGTATCTACCAAAAGAAGCCAACAA | |
| | GCTGTAATCCCGAATATCGGATCTAGACCCAGAATAAGGAATATCCCTAGCA | |
| | GAATAAGCATCTATTGGACAATAGTAAAACCGGGAGACATACTTTTGATTAA | |
| | CAGCACAGGGAATCTAATTGCTCCTAGGGGTTACTTCAAAATACGAAGTGG | |
| | GAAAAGCTCAATAATGAGATCAGATGCACCCATTGGCAAATGCAAGTCTGAA | |
| | TGCATCACTCCAAATGGAAGCATTCCCAATGACAAACCATTCCAAAATGTAA | |
| | ACAGGATCACATACGGGGCCTGTCCCAGATATGTTAAGCAAAGCACTCTGA | |
| | AATTGGCAACAGGAATGCGAAATGTACCAGAGAAACAAACTAGAGGCATATT | |
| | TGGCGCAATAGCGGGTTTCATAGAAAATGGTTGGGAGGGAATGGTGGATG | |
| | GTTGGTACGGTTTCAGGCATCAAAATTCTGAGGGAAGAGGACAAGCAGCAG | |
| | ATCTCAAAAGCACTCAAGCAGCAATCGATCAAATCAATGGGAAGCTGAATCG | |
| | ATTGATCGGGAAAACCAACGAGAAATTCCATCAGATTGAGAAAGAATTCTCA | |
| | GAAGTAGAAGGGAGAATTCAGGACCTTGAGAAATATGTTGAGGACACAAAA | |
| | ATAGATCTCTGGTCATACAACGCAGAGCTTCTTGTTGCCCTGGAAAACCAAC | |
| | ATACAATTGATCTAACTGACTCAGAAATGAACAAACTGTTTGAAAAAACAAAG | |
| | AAGCAACTGAGGGAAAATGCTGAGGATATGGGCAATGGTTGTTTCAAAATAT | |
| | ACCACAAATGTGACAATGCCTGCATAGGATCAATCAGAAATGGAACTTATGA | |
| | CCACGATGTATACAGGGATGAAGCATTAAACAACCGGTTCCAGATCAAGGG | |
| | AGTTGAGCTGAAGTCAGGGTACAAAGATTGGATCCTATGGATTTCCTTTGCC | |
| | ATATCATGTTTTTTGCTTTGTGTTGCTTTGTTGGGGTTCATCATGTGGGCCTG | |
| | CCAAAAGGGCAACATTAGGTGCAACATTTGCATTTGAGTGCATTAATTAAAA | |
| | ACACCCTTGTTTCTACTGGAAGCGGAGCCACGAACTTCTCTCTGTTAAAGCA | |
| | AGCAGGAGATGTTGAAGAAAACCCCGGGCCTATTTTCTAATATCCACAAAAT | |
| | GAAGGCAATAATTGTACTACTCATGGTAGTAACATCCAATGCAGATCGAATC | |
| | TGCACTGGGATAACATCGTCAAACTCACCACATGTCGTCAAAACTGCTACTC | |
| | AAGGGGAGGTCAACGTGACCGGTGTAATACCACTGACAACAACACCCACCA | |
| | AATCTCATTTTGCAAATCTCAAAGGAACAGAAACCAGGGGGAAACTATGCCC | |
| | AAAATGCCTCAACTGCACAGATCTGGATGTAGCCTTGGGCAGACCAAAATG | |
| | CACAGGGAAAATACCCTCTGCAAGGGTTTCAATACTCCATGAAGTCAGACCT | |
| | GTTACATCTGGGTGCTTTCCTATAATGCACGATAGAACAAAAATTAGACAGC | |
| | TGCCTAACCTTCTCCGAGGATACGAACATGTCAGGTTATCAACTCACAACGT | |
| | TATCAATGCAAAAGATGCACCAGGAAGACCCTACGAAATTGGAACCTCAGG | |
| | GTCTTGCCCTAACATTACCAATGGAAACGGATTCTTCGCAACAATGGCTTGG | |
| | GCCGTCCCAAAAAACAAAACAGCAACAAATCCATTAACAATAGAAGTACCAT | |
| | ACATTTGTACAGAAGGAGAAGACCAAATTACCGTTTGGGGGTTCCACTCTGA | |
| | CAACGAGACCCAAATGGCAAAGCTCTATGGGGACTCAAAGCCCCAGAAGTT | |
| | CACCTCATCTGCCAACGGAGTGACCACACATTACGTTTCACAGATTGGTGG | |
| | CTTCCCAAATCAAACAGAAGACGGAGGACTACCACAAAGTGGCAGAATTGT | |
| | TGTTGATTACATGGTGCAGAAATCTGGAAAAACAGGAACAATTACCTATCAA | |
| | AGAGGTATTTTATTGCCTCAAAAAGTGTGGTGCGCAAGTGGCAGGAGCAAG | |
| | GTAATAAAAGGATCCTTGCCCTTAATTGGAGAAGCAGATTGCCTCCATGAAA | |
| | AATACGGTGGATTAAACAAAAGCAAGCCTTACTACACAGGGGAACATGCAA | |
| | AGGCCATAGGAAATTGCCCAATATGGGTGAAAACACCCTTGAAGCTGGCCA | |
| | ATGGAACCAAATATAGACCCCCTGCAAAACTATTAAAGGAAAGAGGTTTCTT | |
| | CGGAGCCATTGCTGGTTTCTTAGAGGGAGGATGGGAAGGAATGATTGCAG | |
| | GTTGGCACGGATACACATCCCATGGGGCACATGGAGTAGCGGTGGCAGCT | |
| | GACCTTAAGAGCACTCAAGAGGCCATAAACAAGATAACAAAAAATCTCAACT | |
| | CTTTGAGTGAGCTGGAAGTAAAGAATCTTCAAAGACTAAGCGGTGCCATGG | |
| | ATGAACTCCACAACGAAATACTAGAACTAGATGAGAAAGTGGATGATCTCAG | |
| | AGCTGACACAATAAGCTCACAAATAGAACTCGCAGTCCTGCTTTCCAATGAA | |
| | GGAATAATAAACAGTGAAGATGAACATCTCTTGGCGCTTGAAAGAAAGCTGA | |
| | AGAAAATGCTGGGCCCCTCTGCTGTAGAGATAGGGAATGGATGCTTTGAAA | |
| | CCAAACACAAGTGCAACCAGACCTGCCTCGACAGAATAGCTGCTGGTACCT | |
| | TTGATGCAGGAGAATTTTCTCTCCCCACCTTTGATTCACTGAATATTACTGCT | |
| | GCATCTTTAAATGACGACGGATTGGACAATCATACTATACTGCTTTACTACTC | |
| | AACTGCTGCCTCCAGTTTGGCTGTAACACTGATGATAGCTATCTTTGTTGTT | |
| | TATATGGTCTCCAGAGACAATGTTTCTTGCTCCATTTGTCTATAAGGAAAGTT | |
| | AAGCCCTGTATTTTCCTTTATTGTAGTGCTTGTTTGCTTGTTGTCATTACAAA | |
| | GAAACGTTATTGAAAAATTGATGACTCGAGCTGGTACTGCATGCACGCAATG | |
| | CTAGCTGCCCCTTTCCCGTCCTGGGTACCCCGAGTCTCCCCCGACCTCGG | |
| | GTCCCAGGTATGCTCCCACCTCCACCTGCCCCACTCACCACCTCTGCTAGT | |
| | TCCAGACACCTCCCAAGCACGCAGCAATGCAGCTCAAAACGCTTAGCCTAG | |
| | CCACACCCCCACGGGAAACAGCAGTGATTAACCTTTAGCAATAAACGAAAG | |
| | TTTAACTAAGCTATACTAACCCCAGGGTTGGTCAATTTCGTGCCAGCCACAC | |
| | CCTGGAGCTAGCAAAAAAAA | |
| Pan-CoV- | CTCGACATTGATTATTGACTAGTTATTAATAGTAATCAATTACGGGGTCATTA | 42 |
| Flu | GTTCATAGCCCATATATGGAGTTCCGCGTTACATAACTTACGGTAAATGGCC | |
| Vaccine | CGCCTGGCTGACCGCCCAACGACCCCCGCCCATTGACGTCAATAATGACGT | |
| #22 CoV- | ATGTTCCCATAGTAACGCCAATAGGGACTTTCCATTGACGTCAATGGGTGGA | |
| Wuhan | GTATTTACGGTAAACTGCCCACTTGGCAGTACATCAAGTGTATCATATGCCA | |
| Spike | AGTACGCCCCCTATTGACGTCAATGACGGTAAATGGCCCGCCTGGCATTAT | |
| (2 proline | GCCCAGTACATGACCTTATGGGACTTTCCTACTTGGCAGTACATCTACGTAT | |
| subst.)- | TAGTCATCGCTATTACCATGGTCGAGGTGAGCCCCACGTTCTGCTTCACTCT | |
| Influenza | CCCCATCTCCCCCCCCTCCCCACCCCCAATTTTGTATTTATTTATTTTTTAAT | |
| (HA) | TATTTTGTGCAGCGATGGGGGCGGGGGGGGGGGGGGGGCGCGCGCCAG | |
| | GCGGGGCGGGGCGGGGCGAGGGGCGGGGCGGGGCGAGGCGGAGAGGT | |
| | GCGGCGGCAGCCAATCAGAGCGGCGCGCTCCGAAAGTTTCCTTTTATGGC | |
| | GAGGCGGCGGCGGCGGCGGCCCTATAAAAAGCGAAGCGCGCGGCGGGGC | |
| | GGGAGTCGCTGCGCGCTGCCTTCGCCCCGTGCCCCGCTCCGCCGCCGCC | |
| | TCGCGCCGCCCGCCCCGGCTCTGACTGACCGCGTTACTCCCACAGGTGAG | |
| | CGGGCGGGACGGCCCTTCTCCTCCGGGCTGTAATTAGCGCTTGGTTTAATG | |
| | ACGGCTTGTTTCTTTTCTGTGGCTGCGTGAAAGCCTTGAGGGGCTCCGGGA | |
| | GGGCCCTTTGTGCGGGGGGAGCGGCTCGGGGGGTGCGTGCGTGTGTGTG | |
| | TGCGTGGGGAGCGCCGCGTGCGGCTCCGCGCTGCCCGGCGGCTGTGAGC | |
| | GCTGCGGGCGCGGCGCGGGGCTTTGTGCGCTCCGCAGTGTGCGCGAGGG | |
| | GAGCGCGGCCGGGGGCGGTGCCCCGCGGTGCGGGGGGGGCTGCGAGGG | |
| | GAACAAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCCCGAGT | |
| | TGCTGAGCACGGCCCGGCTTCGGGTGCGGGGCTCCGTACGGGGCGTGGC | |
| | GCGGGGCTCGCCGTGCCGGGCGGGGGGTGGCGGCAGGTGGGGGTGCCG | |
| | GGCGGGGCGGGGCCGCCTCGGGCCGGGGAGGGCTCGGGGGAGGGGCG | |
| | CGGCGGCCCCCGGAGCGCCGGCGGCTGTCGAGGCGCGGCGAGCCGCAG | |
| | CCATTGCCTTTTATGGTAATCGTGCGAGAGGGCGCAGGGACTTCCTTTGTC | |
| | CCAAATCTGTGCGGAGCCGAAATCTGGGAGGCGCCGCCGCACCCCCTCTA | |
| | GCGGGCGCGGGGCGAAGCGGTGCGGCGCCGGCAGGAAGGAAATGGGCG | |
| | GGGAGGGCCTTCGTGCGTCGCCGCGCCGCCGTCCCCTTCTCCCTCTCCAG | |
| | CCTCGGGGCTGTCCGGGGGGGGACGGCTGCCTTCGGGGGGGACGGGGC | |
| | AGGGGGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGTGCTG | |
| | GTTATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTTCCT | |
| | GGTGCTGCTGCCCCTGGTGAGCAGCCAGTGCGTGAACCTGACCACCAGGA | |
| | CCCAGCTGCCCCCCGCCTACACCAACAGCTTCACCAGGGGCGTGTACTAC | |
| | CCCGACAAGGTGTTCAGGAGCAGCGTGCTGCACAGCACCCAGGACCTGTT | |
| | CCTGCCCTTCTTCAGCAACGTGACCTGGTTCCACGCCATCCACGTGAGCGG | |
| | CACCAACGGCACCAAGAGGTTCGACAACCCCGTGCTGCCCTTCAACGACG | |
| | GCGTGTACTTCGCCAGCACCGAGAAGAGCAACATCATCAGGGGCTGGATCT | |
| | TCGGCACCACCCTGGACAGCAAGACCCAGAGCCTGCTGATCGTGAACAAC | |
| | GCCACCAACGTGGTGATCAAGGTGTGCGAGTTCCAGTTCTGCAACGACCCC | |
| | TTCCTGGGCGTGTACTACCACAAGAACAACAAGAGCTGGATGGAGAGCGAG | |
| | TTCAGGGTGTACAGCAGCGCCAACAACTGCACCTTCGAGTACGTGAGCCAG | |
| | CCCTTCCTGATGGACCTGGAGGGCAAGCAGGGCAACTTCAAGAACCTGAG | |
| | GGAGTTCGTGTTCAAGAACATCGACGGCTACTTCAAGATCTACAGCAAGCA | |
| | CACCCCCATCAACCTGGTGAGGGACCTGCCCCAGGGCTTCAGCGCCCTGG | |
| | AGCCCCTGGTGGACCTGCCCATCGGCATCAACATCACCAGGTTCCAGACCC | |
| | TGCTGGCCCTGCACAGGAGCTACCTGACCCCCGGCGACAGCAGCAGCGGC | |
| | TGGACCGCCGGCGCCGCCGCCTACTACGTGGGCTACCTGCAGCCCAGGAC | |
| | CTTCCTGCTGAAGTACAACGAGAACGGCACCATCACCGACGCCGTGGACTG | |
| | CGCCCTGGACCCCCTGAGCGAGACCAAGTGCACCCTGAAGAGCTTCACCG | |
| | TGGAGAAGGGCATCTACCAGACCAGCAACTTCAGGGTGCAGCCCACCGAG | |
| | AGCATCGTGAGGTTCCCCAACATCACCAACCTGTGCCCCTTCGGCGAGGTG | |
| | TTCAACGCCACCAGGTTCGCCAGCGTGTACGCCTGGAACAGGAAGAGGAT | |
| | CAGCAACTGCGTGGCCGACTACAGCGTGCTGTACAACAGCGCCAGCTTCA | |
| | GCACCTTCAAGTGCTACGGCGTGAGCCCCACCAAGCTGAACGACCTGTGCT | |
| | TCACCAACGTGTACGCCGACAGCTTCGTGATCAGGGGCGACGAGGTGAGG | |
| | CAGATCGCCCCCGGCCAGACCGGCAAGATCGCCGACTACAACTACAAGCT | |
| | GCCCGACGACTTCACCGGCTGCGTGATCGCCTGGAACAGCAACAACCTGG | |
| | ACAGCAAGGTGGGGGGCAACTACAACTACCTGTACAGGCTGTTCAGGAAGA | |
| | GCAACCTGAAGCCCTTCGAGAGGGACATCAGCACCGAGATCTACCAGGCC | |
| | GGCAGCACCCCCTGCAACGGCGTGGAGGGCTTCAACTGCTACTTCCCCCT | |
| | GCAGAGCTACGGCTTCCAGCCCACCAACGGCGTGGGCTACCAGCCCTACA | |
| | GGGTGGTGGTGCTGAGCTTCGAGCTGCTGCACGCCCCCGCCACCGTGTGC | |
| | GGCCCCAAGAAGAGCACCAACCTGGTGAAGAACAAGTGCGTGAACTTCAAC | |
| | TTCAACGGCCTGACCGGCACCGGCGTGCTGACCGAGAGCAACAAGAAGTT | |
| | CCTGCCCTTCCAGCAGTTCGGCAGGGACATCGCCGACACCACCGACGCCG | |
| | TGAGGGACCCCCAGACCCTGGAGATCCTGGACATCACCCCCTGCAGCTTC | |
| | GGCGGCGTGAGCGTGATCACCCCCGGCACCAACACCAGCAACCAGGTGGC | |
| | CGTGCTGTACCAGGACGTGAACTGCACCGAGGTGCCCGTGGCCATCCACG | |
| | CCGACCAGCTGACCCCCACCTGGAGGGTGTACAGCACCGGCAGCAACGTG | |
| | TTCCAGACCAGGGCCGGCTGCCTGATCGGCGCCGAGCACGTGAACAACAG | |
| | CTACGAGTGCGACATCCCCATCGGCGCCGGCATCTGCGCCAGCTACCAGA | |
| | CCCAGACCAACAGCCCCAGGAGGGCCAGGAGCGTGGCCAGCCAGAGCAT | |
| | CATCGCCTACACCATGAGCCTGGGCGCCGAGAACAGCGTGGCCTACAGCA | |
| | ACAACAGCATCGCCATCCCCACCAACTTCACCATCAGCGTGACCACCGAGA | |
| | TCCTGCCCGTGAGCATGACCAAGACCAGCGTGGACTGCACCATGTACATCT | |
| | GCGGCGACAGCACCGAGTGCAGCAACCTGCTGCTGCAGTACGGCAGCTTC | |
| | TGCACCCAGCTGAACAGGGCCCTGACCGGCATCGCCGTGGAGCAGGACAA | |
| | GAACACCCAGGAGGTGTTCGCCCAGGTGAAGCAGATCTACAAGACCCCCC | |
| | CCATCAAGGACTTCGGCGGCTTCAACTTCAGCCAGATCCTGCCCGACCCCA | |
| | GCAAGCCCAGCAAGAGGAGCTTCATCGAGGACCTGCTGTTCAACAAGGTGA | |
| | CCCTGGCCGACGCCGGCTTCATCAAGCAGTACGGCGACTGCCTGGGCGAC | |
| | ATCGCCGCCAGGGACCTGATCTGCGCCCAGAAGTTCAACGGCCTGACCGT | |
| | GCTGCCCCCCCTGCTGACCGACGAGATGATCGCCCAGTACACCAGCGCCC | |
| | TGCTGGCCGGCACCATCACCAGCGGCTGGACCTTCGGCGCCGGCGCCGC | |
| | CCTGCAGATCCCCTTCGCCATGCAGATGGCCTACAGGTTCAACGGCATCGG | |
| | CGTGACCCAGAACGTGCTGTACGAGAACCAGAAGCTGATCGCCAACCAGTT | |
| | CAACAGCGCCATCGGCAAGATCCAGGACAGCCTGAGCAGCACCGCCAGCG | |
| | CCCTGGGCAAGCTGCAGGACGTGGTGAACCAGAACGCCCAGGCCCTGAAC | |
| | ACCCTGGTGAAGCAGCTGAGCAGCAACTTCGGCGCCATCAGCAGCGTGCT | |
| | GAACGACATCCTGAGCAGGCTGGACCCCCCCGAGGCCGAGGTGCAGATCG | |
| | ACAGGCTGATCACCGGCAGGCTGCAGAGCCTGCAGACCTACGTGACCCAG | |
| | CAGCTGATCAGGGCCGCCGAGATCAGGGCCAGCGCCAACCTGGCCGCCA | |
| | CCAAGATGAGCGAGTGCGTGCTGGGCCAGAGCAAGAGGGTGGACTTCTGC | |
| | GGCAAGGGCTACCACCTGATGAGCTTCCCCCAGAGCGCCCCCCACGGCGT | |
| | GGTGTTCCTGCACGTGACCTACGTGCCCGCCCAGGAGAAGAACTTCACCAC | |
| | CGCCCCCGCCATCTGCCACGACGGCAAGGCCCACTTCCCCAGGGAGGGC | |
| | GTGTTCGTGAGCAACGGCACCCACTGGTTCGTGACCCAGAGGAACTTCTAC | |
| | GAGCCCCAGATCATCACCACCGACAACACCTTCGTGAGCGGCAACTGCGA | |
| | CGTGGTGATCGGCATCGTGAACAACACCGTGTACGACCCCCTGCAGCCCG | |
| | AGCTGGACAGCTTCAAGGAGGAGCTGGACAAGTACTTCAAGAACCACACCA | |
| | GCCCCGACGTGGACCTGGGCGACATCAGCGGCATCAACGCCAGCGTGGTG | |
| | AACATCCAGAAGGAGATCGACAGGCTGAACGAGGTGGCCAAGAACCTGAA | |
| | CGAGAGCCTGATCGACCTGCAGGAGCTGGGCAAGTACGAGCAGTACATCA | |
| | AGTGGCCCTGGTACATCTGGCTGGGCTTCATCGCCGGCCTGATCGCCATC | |
| | GTGATGGTGACCATCATGCTGTGCTGCATGACCAGCTGCTGCAGCTGCCTG | |
| | AAGGGCTGCTGCAGCTGCGGCAGCTGCTGCAAGTTCGACGAGGACGACAG | |
| | CGAGCCCGTGCTGAAGGGCGTGAAGCTGCACTACACCGGAAGCGGAGCCA | |
| | CGAACTTCTCTCTGTTAAAGCAAGCAGGAGATGTTGAAGAAAACCCCGGGC | |
| | CTATGAAGGCAATACTAGTAGTTCTGCTATATACATTTGCAACCGCAAATGC | |
| | AGACACATTATGTATAGGTTATCATGCGAACAACTCAACAGACACTGTAGAC | |
| | ACAGTACTAGAAAAGAATGTAACAGTAACACACTCTGTTAACCTTCTAGAAG | |
| | ACAAGCATAACGGGAAACTATGCAAACTAAGAGGGGTAGCCCCATTGCATT | |
| | TGGGTAAATGTAACATTGCTGGCTGGATCCTGGGAAATCCAGAGTGTGAAT | |
| | CACTCTCCACAGCAAGCTCATGGTCCTACATTGTGGAAACATCTAGTTCAGA | |
| | CAATGGAACGTGTTACCCAGGAGATTTCATCGATTATGAGGAGCTAAGAGA | |
| | GCAATTGAGCTCAGTGTCATCATTTGAAAGGTTTGAGATATTCCCCAAGACA | |
| | AGTTCATGGCCCAATCATGACTCGAACAAAGGTGTAACGACAGCATGTCCT | |
| | CATGCTGGAGCAAAAAGCTTCTACAAAAATTTAATATGGCTAGTTAAAAAAG | |
| | GAAATTCATACCCAAAGCTCAGCAAATCCTACATTAATGATAAAGGGAAAGA | |
| | AGTCCTCGTGCTATGGGGCATTCACCATCCACCTACTAGTGCTGACCAACA | |
| | AAGTCTCTATCAGAATGCAGATGCATATGTTTTTGTGGGGACATCAAGATAC | |
| | AGCAAGAAGTTCAAGCCGGAAATAGCAATAAGGCCCAAAGTGAGGGATCAA | |
| | GAAGGGAGAATGAACTATTACTGGACACTAGTAGAGCCGGGAGACAAAATA | |
| | ACATTCGAAGCAACTGGAAATCTAGTGGTACCGAGATATGCATTCGCAATG | |
| | GAAAGAAATGCTGGATCTGGTATTATCATTTCAGATACACCAGTCCACGATT | |
| | GCAATACAACTTGTCAGACACCCAAGGGTGCTATAAACACCAGCCTCCCATT | |
| | TCAGAACATACATCCGATCACAATTGGAAAATGTCCAAAATATGTAAAAAGC | |
| | ACAAAATTGAGACTGGCCACAGGATTGAGGAATGTCCCGTCCATTCAATCTA | |
| | GAGGCCTATTTGGGGCCATTGCCGGTTTCATTGAAGGGGGGTGGACAGGG | |
| | ATGGTAGATGGATGGTACGGTTATCACCATCAAAATGAGCAGGGGTCAGGA | |
| | TATGCAGCCGACCTGAAGAGCACACAGAATGCCATTGACGAGATTACTAAC | |
| | AAAGTAAACTCTGTTATTGAAAAAATGAATACACAGTTCACAGCAGTAGGTA | |
| | AAGAGTTCAACCACCTGGAAAAAAGAATAGAGAATTTAAATAAAAAAGTTGA | |
| | TGATGGTTTCCTGGACATTTGGATTACAATGCCGAACTGTTGGTTCTATTG | |
| | GAAAATGAAAGAACTTTGGACTACCACGATTCAAAGGTGAAGAACTTATATG | |
| | AAAAGGTAAGAAGCCAGTTAAAAAACAATGCCAAGGAAATTGGAAACGGCT | |
| | GCTTTGAATTTTACCACAAATGTGATAACACGTGCATGGAAAGTGTCAAAAA | |
| | TGGGACTTATGACTACCCAAAATACTCAGAGGAAGCAAAATTAAACAGAGAA | |
| | GAAATAGATGGGGTAAAGCTGGAATCAACAAGGATTTACCAGATTTTGGCG | |
| | ATCTATTCAACCGTCGCCAGTTCATTGGTACTGGTAGTCTCCCTGGGGGCA | |
| | ATCAGTTTCTGGATGTGCTCTAATGGGTCTCTACAGTGTAGAATATGTATTTA | |
| | AGGAAGCGGAGCCACGAACTTCTCTCTGTTAAAGCAAGCAGGAGATGTTGA | |
| | AGAAAACCCCGGGCCTAGCAAAAGCAGGGGATAATTCTATTAACCATGAAG | |
| | ACTATCATTGCTTTGAGCTACATTCTATGTCTGGTTTTCGCTCAAAAACTTCC | |
| | TGGAAATGACAATAGCACTGCAACGCTGTGCCTTGGGCACCATGCAGTACC | |
| | AAACGGAACGATAGTGAAAACAATCACGAATGACCAAATTGAAGTTACTAAT | |
| | GCTACTGAGCTGGTTCAGAATTCCTCAATAGGTGAAATATGCGACAGTCCTC | |
| | ATCAGATCCTTGATGGAGAAAACTGCACACTAATAGATGCTCTATTGGGAGA | |
| | CCCTCAGTGTGATGGCTTTCAAAATAAGAAATGGGACCTTTTTGTTGAACGA | |
| | AGCAAAGCCTACAGCAACTGTTACCCTTATGATGTGCCGGATTATGCCTCCC | |
| | TTAGGTCACTAGTTGCCTCATCCGGCACACTGGAGTTTAACAATGAAAGCTT | |
| | CAATTGGGCTGGAGTCACTCAAAACGGAACAAGTTCTGCTTGCATAAGGGG | |
| | ATCTAATAGTAGTTTCTTTAGTAGATTAAATTGGTTGACCCACTTAAACTTCA | |
| | AGTACCCAGCATTGAACGTGACTATGCCAAACAATGAACAATTTGACAAATT | |
| | GTACATTTGGGGGGTTCACCACCCGGGTACGGACAAGGACCAAATCTTCCT | |
| | GTATGCTCAATCATCAGGAAGAATCACAGTATCTACCAAAAGAAGCCAACAA | |
| | GCTGTAATCCCGAATATCGGATCTAGACCCAGAATAAGGAATATCCCTAGCA | |
| | GAATAAGCATCTATTGGACAATAGTAAAACCGGGAGACATACTTTTGATTAA | |
| | CAGCACAGGGAATCTAATTGCTCCTAGGGGTTACTTCAAAATACGAAGTGG | |
| | GAAAAGCTCAATAATGAGATCAGATGCACCCATTGGCAAATGCAAGTCTGAA | |
| | TGCATCACTCCAAATGGAAGCATTCCCAATGACAAACCATTCCAAAATGTAA | |
| | ACAGGATCACATACGGGGCCTGTCCCAGATATGTTAAGCAAAGCACTCTGA | |
| | AATTGGCAACAGGAATGCGAAATGTACCAGAGAAACAAACTAGAGGCATATT | |
| | TGGCGCAATAGCGGGTTTCATAGAAAATGGTTGGGAGGGAATGGTGGATG | |
| | GTTGGTACGGTTTCAGGCATCAAAATTCTGAGGGAAGAGGACAAGCAGCAG | |
| | ATCTCAAAAGCACTCAAGCAGCAATCGATCAAATCAATGGGAAGCTGAATCG | |
| | ATTGATCGGGAAAACCAACGAGAAATTCCATCAGATTGAGAAAGAATTCTCA | |
| | GAAGTAGAAGGGAGAATTCAGGACCTTGAGAAATATGTTGAGGACACAAAA | |
| | ATAGATCTCTGGTCATACAACGCAGAGCTTCTTGTTGCCCTGGAAAACCAAC | |
| | ATACAATTGATCTAACTGACTCAGAAATGAACAAACTGTTTGAAAAAACAAAG | |
| | AAGCAACTGAGGGAAAATGCTGAGGATATGGGCAATGGTTGTTTCAAAATAT | |
| | ACCACAAATGTGACAATGCCTGCATAGGATCAATCAGAAATGGAACTTATGA | |
| | CCACGATGTATACAGGGATGAAGCATTAAACAACCGGTTCCAGATCAAGGG | |
| | AGTTGAGCTGAAGTCAGGGTACAAAGATTGGATCCTATGGATTTCCTTTGCC | |
| | ATATCATGTTTTTTGCTTTGTGTTGCTTTGTTGGGGTTCATCATGTGGGCCTG | |
| | CCAAAAGGGCAACATTAGGTGCAACATTTGCATTTGAGTGCATTAATTAAAA | |
| | ACACCCTTGTTTCTACTGGAAGCGGAGCCACGAACTTCTCTCTGTTAAAGCA | |
| | AGCAGGAGATGTTGAAGAAAACCCCGGGCCTATTTTCTAATATCCACAAAAT | |
| | GAAGGCAATAATTGTACTACTCATGGTAGTAACATCCAATGCAGATCGAATC | |
| | TGCACTGGGATAACATCGTCAAACTCACCACATGTCGTCAAAACTGCTACTC | |
| | AAGGGGAGGTCAACGTGACCGGTGTAATACCACTGACAACAACACCCACCA | |
| | AATCTCATTTTGCAAATCTCAAAGGAACAGAAACCAGGGGGAAACTATGCCC | |
| | AAAATGCCTCAACTGCACAGATCTGGATGTAGCCTTGGGCAGACCAAAATG | |
| | CACAGGGAAAATACCCTCTGCAAGGGTTTCAATACTCCATGAAGTCAGACCT | |
| | GTTACATCTGGGTGCTTTCCTATAATGCACGATAGAACAAAAATTAGACAGC | |
| | TGCCTAACCTTCTCCGAGGATACGAACATGTCAGGTTATCAACTCACAACGT | |
| | TATCAATGCAAAAGATGCACCAGGAAGACCCTACGAAATTGGAACCTCAGG | |
| | GTCTTGCCCTAACATTACCAATGGAAACGGATTCTTCGCAACAATGGCTTGG | |
| | GCCGTCCCAAAAAACAAAACAGCAACAAATCCATTAACAATAGAAGTACCAT | |
| | ACATTTGTACAGAAGGAGAAGACCAAATTACCGTTTGGGGGTTCCACTCTGA | |
| | CAACGAGACCCAAATGGCAAAGCTCTATGGGGACTCAAAGCCCCAGAAGTT | |
| | CACCTCATCTGCCAACGGAGTGACCACACATTACGTTTCACAGATTGGTGG | |
| | CTTCCCAAATCAAACAGAAGACGGAGGACTACCACAAAGTGGCAGAATTGT | |
| | TGTTGATTACATGGTGCAGAAATCTGGAAAAACAGGAACAATTACCTATCAA | |
| | AGAGGTATTTTATTGCCTCAAAAAGTGTGGTGCGCAAGTGGCAGGAGCAAG | |
| | GTAATAAAAGGATCCTTGCCCTTAATTGGAGAAGCAGATTGCCTCCATGAAA | |
| | AATACGGTGGATTAAACAAAAGCAAGCCTTACTACACAGGGGAACATGCAA | |
| | AGGCCATAGGAAATTGCCCAATATGGGTGAAAACACCCTTGAAGCTGGCCA | |
| | ATGGAACCAAATATAGACCCCCTGCAAAACTATTAAAGGAAAGAGGTTTCTT | |
| | CGGAGCCATTGCTGGTTTCTTAGAGGGAGGATGGGAAGGAATGATTGCAG | |
| | GTTGGCACGGATACACATCCCATGGGGCACATGGAGTAGCGGTGGCAGCT | |
| | GACCTTAAGAGCACTCAAGAGGCCATAAACAAGATAACAAAAAATCTCAACT | |
| | CTTTGAGTGAGCTGGAAGTAAAGAATCTTCAAAGACTAAGCGGTGCCATGG | |
| | ATGAACTCCACAACGAAATACTAGAACTAGATGAGAAAGTGGATGATCTCAG | |
| | AGCTGACACAATAAGCTCACAAATAGAACTCGCAGTCCTGCTTTCCAATGAA | |
| | GGAATAATAAACAGTGAAGATGAACATCTCTTGGCGCTTGAAAGAAAGCTGA | |
| | AGAAAATGCTGGGCCCCTCTGCTGTAGAGATAGGGAATGGATGCTTTGAAA | |
| | CCAAACACAAGTGCAACCAGACCTGCCTCGACAGAATAGCTGCTGGTACCT | |
| | TTGATGCAGGAGAATTTTCTCTCCCCACCTTTGATTCACTGAATATTACTGCT | |
| | GCATCTTTAAATGACGACGGATTGGACAATCATACTATACTGCTTTACTACTC | |
| | AACTGCTGCCTCCAGTTTGGCTGTAACACTGATGATAGCTATCTTTGTTGTT | |
| | TATATGGTCTCCAGAGACAATGTTTCTTGCTCCATTTGTCTATAAGGAAAGTT | |
| | AAGCCCTGTATTTTCCTTTATTGTAGTGCTTGTTTGCTTGTTGTCATTACAAA | |
| | GAAACGTTATTGAAAAATTGATGACTCGAGCTGGTACTGCATGCACGCAATG | |
| | CTAGCTGCCCCTTTCCCGTCCTGGGTACCCCGAGTCTCCCCCGACCTCGG | |
| | GTCCCAGGTATGCTCCCACCTCCACCTGCCCCACTCACCACCTCTGCTAGT | |
| | TCCAGACACCTCCCAAGCACGCAGCAATGCAGCTCAAAACGCTTAGCCTAG | |
| | CCACACCCCCACGGGAAACAGCAGTGATTAACCTTTAGCAATAAACGAAAG | |
| | TTTAACTAAGCTATACTAACCCCAGGGTTGGTCAATTTCGTGCCAGCCACAC | |
| | CCTGGAGCTAGCAAAAAAAA | |
| Pan-CoV- | ATGTTCGTGTTCCTGGTGCTGCTGCCCCTGGTGAGCAGCCAGTGCGTGAAC | 43 |
| Flu | CTGATCACCAGGACCCAGAGCTACACCAACAGCTTCACCAGGGGCGTGTAC | |
| Vaccine | TACCCCGACAAGGTGTTCAGGAGCAGCGTGCTGCACAGCACCCAGGACCT | |
| #23 CoV- | GTTCCTGCCCTTCTTCAGCAACGTGACCTGGTTCCACGCCATCCACGTGAG | |
| Omicron | CGGCACCAACGGCACCAAGAGGTTCGACAACCCCGTGCTGCCCTTCAACG | |
| BA.2 Spike | ACGGCGTGTACTTCGCCAGCACCGAGAAGAGCAACATCATCAGGGGCTGG | |
| (HexaPro)- | ATCTTCGGCACCACCCTGGACAGCAAGACCCAGAGCCTGCTGATCGTGAAC | |
| Influenza | AACGCCACCAACGTGGTGATCAAGGTGTGCGAGTTCCAGTTCTGCAACGAC | |
| (HA) | CCCTTCCTGGACGTGTACTACCACAAGAACAACAAGAGCTGGATGGAGAGC | |
| | GAGTTCAGGGTGTACAGCAGCGCCAACAACTGCACCTTCGAGTACGTGAGC | |
| | CAGCCCTTCCTGATGGACCTGGAGGGCAAGCAGGGCAACTTCAAGAACCT | |
| | GAGGGAGTTCGTGTTCAAGAACATCGACGGCTACTTCAAGATCTACAGCAA | |
| | GCACACCCCCATCAACCTGGGCAGGGACCTGCCCCAGGGCTTCAGCGCCC | |
| | TGGAGCCCCTGGTGGACCTGCCCATCGGCATCAACATCACCAGGTTCCAGA | |
| | CCCTGCTGGCCCTGCACAGGAGCTACCTGACCCCCGGCGACAGCAGCAGC | |
| | GGCTGGACCGCCGGCGCCGCCGCCTACTACGTGGGCTACCTGCAGCCCA | |
| | GGACCTTCCTGCTGAAGTACAACGAGAACGGCACCATCACCGACGCCGTG | |
| | GACTGCGCCCTGGACCCCCTGAGCGAGACCAAGTGCACCCTGAAGAGCTT | |
| | CACCGTGGAGAAGGGCATCTACCAGACCAGCAACTTCAGGGTGCAGCCCA | |
| | CCGAGAGCATCGTGAGGTTCCCCAACATCACCAACCTGTGCCCCTTCGACG | |
| | AGGTGTTCAACGCCACCAGGTTCGCCAGCGTGTACGCCTGGAACAGGAAG | |
| | AGGATCAGCAACTGCGTGGCCGACTACAGCGTGCTGTACAACTTCGCCCCC | |
| | TTCTTCGCCTTCAAGTGCTACGGCGTGAGCCCCACCAAGCTGAACGACCTG | |
| | TGCTTCACCAACGTGTACGCCGACAGCTTCGTGATCAGGGGCAACGAGGT | |
| | GAGCCAGATCGCCCCCGGCCAGACCGGCAACATCGCCGACTACAACTACA | |
| | AGCTGCCCGACGACTTCACCGGCTGCGTGATCGCCTGGAACAGCAACAAG | |
| | CTGGACAGCAAGGTGGGCGGCAACTACAACTACCTGTACAGGCTGTTCAG | |
| | GAAGAGCAACCTGAAGCCCTTCGAGAGGGACATCAGCACCGAGATCTACCA | |
| | GGCCGGCAACAAGCCCTGCAACGGCGTGGCCGGCTTCAACTGCTACTTCC | |
| | CCCTGAGGAGCTACGGCTTCAGGCCCACCTACGGCGTGGGCCACCAGCCC | |
| | TACAGGGTGGTGGTGCTGAGCTTCGAGCTGCTGCACGCCCCCGCCACCGT | |
| | GTGCGGCCCCAAGAAGAGCACCAACCTGGTGAAGAACAAGTGCGTGAACT | |
| | TCAACTTCAACGGCCTGACCGGCACCGGCGTGCTGACCGAGAGCAACAAG | |
| | AAGTTCCTGCCCTTCCAGCAGTTCGGCAGGGACATCGCCGACACCACCGA | |
| | CGCCGTGAGGGACCCCCAGACCCTGGAGATCCTGGACATCACCCCCTGCA | |
| | GCTTCGGCGGCGTGAGCGTGATCACCCCCGGCACCAACACCAGCAACCAG | |
| | GTGGCCGTGCTGTACCAGGGCGTGAACTGCACCGAGGTGCCCGTGGCCAT | |
| | CCACGCCGACCAGCTGACCCCCACCTGGAGGGTGTACAGCACCGGCAGCA | |
| | ACGTGTTCCAGACCAGGGCCGGCTGCCTGATCGGCGCCGAGTACGTGAAC | |
| | AACAGCTACGAGTGCGACATCCCCATCGGCGCCGGCATCTGCGCCAGCTA | |
| | CCAGACCCAGACCAAGAGCCACCAGCAGGCCCAGAGCGTGGCCAGCCAGA | |
| | GCATCATCGCCTACACCATGAGCCTGGGCGCCGAGAACAGCGTGGCCTAC | |
| | AGCAACAACAGCATCGCCATCCCCACCAACTTCACCATCAGCGTGACCACC | |
| | GAGATCCTGCCCGTGAGCATGACCAAGACCAGCGTGGACTGCACCATGTA | |
| | CATCTGCGGCGACAGCACCGAGTGCAGCAACCTGCTGCTGCAGTACGGCA | |
| | GCTTCTGCACCCAGCTGAAGAGGGCCCTGACCGGCATCGCCGTGGAGCAG | |
| | GACAAGAACACCCAGGAGGTGTTCGCCCAGGTGAAGCAGATCTACAAGAC | |
| | CCCCCCCATCAAGTACTTCGGCGGCTTCAACTTCAGCCAGATCCTGCCCGA | |
| | CCCCAGCAAGCCCAGCAAGAGGAGCCCCATCGAGGACCTGCTGTTCAACA | |
| | AGGTGACCCTGGCCGACGCCGGCTTCATCAAGCAGTACGGCGACTGCCTG | |
| | GGCGACATCGCCGCCAGGGACCTGATCTGCGCCCAGAAGTTCAACGGCCT | |
| | GACCGTGCTGCCCCCCCTGCTGACCGACGAGATGATCGCCCAGTACACCA | |
| | GCGCCCTGCTGGCCGGCACCATCACCAGCGGCTGGACCTTCGGCGCCGG | |
| | CCCCGCCCTGCAGATCCCCTTCCCCATGCAGATGGCCTACAGGTTCAACGG | |
| | CATCGGCGTGACCCAGAACGTGCTGTACGAGAACCAGAAGCTGATCGCCA | |
| | ACCAGTTCAACAGCGCCATCGGCAAGATCCAGGACAGCCTGAGCAGCACC | |
| | CCCAGCGCCCTGGGCAAGCTGCAGGACGTGGTGAACCACAACGCCCAGGC | |
| | CCTGAACACCCTGGTGAAGCAGCTGAGCAGCAAGTTCGGCGCCATCAGCA | |
| | GCGTGCTGAACGACATCCTGAGCAGGCTGGACCCCCCCGAGGCCGAGGTG | |
| | CAGATCGACAGGCTGATCACCGGCAGGCTGCAGAGCCTGCAGACCTACGT | |
| | GACCCAGCAGCTGATCAGGGCCGCCGAGATCAGGGCCAGCGCCAACCTG | |
| | GCCGCCACCAAGATGAGCGAGTGCGTGCTGGGCCAGAGCAAGAGGGTGG | |
| | ACTTCTGCGGCAAGGGCTACCACCTGATGAGCTTCCCCCAGAGCGCCCCC | |
| | CACGGCGTGGTGTTCCTGCACGTGACCTACGTGCCCGCCCAGGAGAAGAA | |
| | CTTCACCACCGCCCCCGCCATCTGCCACGACGGCAAGGCCCACTTCCCCA | |
| | GGGAGGGCGTGTTCGTGAGCAACGGCACCCACTGGTTCGTGACCCAGAGG | |
| | AACTTCTACGAGCCCCAGATCATCACCACCGACAACACCTTCGTGAGCGGC | |
| | AACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAA | |
| | CCACACCAGCCCCGACGTGGACCTGGGCGACATCAGCGGCATCAACGCCA | |
| | GCGTGGTGAACATCCAGAAGGAGATCGACAGGCTGAACGAGGTGGCCAAG | |
| | AACCTGAACGAGAGCCTGATCGACCTGCAGGAGCTGGGCAAGTACGAGCA | |
| | GTACATCAAGTGGCCCTGGTACATCTGGCTGGGCTTCATCGCCGGCCTGAT | |
| | CGCCATCGTGATGGTGACCATCATGCTGTGCTGCATGACCAGCTGCTGCAG | |
| | CTGCCTGAAGGGCTGCTGCAGCTGCGGCAGCTGCTGCAAGTTCGACGAGG | |
| | ACGACAGCGAGCCCGTGCTGAAGGGCGTGAAGCTGCACTACACCGGAAGC | |
| | GGAGCCACGAACTTCTCTCTGTTAAAGCAAGCAGGAGATGTTGAAGAAAAC | |
| | CCCGGGCCTATGAAGGCAATACTAGTAGTTCTGCTATATACATTTGCAACCG | |
| | CAAATGCAGACACATTATGTATAGGTTATCATGCGAACAACTCAACAGACAC | |
| | TGTAGACACAGTACTAGAAAAGAATGTAACAGTAACACACTCTGTTAACCTT | |
| | CTAGAAGACAAGCATAACGGGAAACTATGCAAACTAAGAGGGGTAGCCCCA | |
| | TTGCATTTGGGTAAATGTAACATTGCTGGCTGGATCCTGGGAAATCCAGAGT | |
| | GTGAATCACTCTCCACAGCAAGCTCATGGTCCTACATTGTGGAAACATCTAG | |
| | TTCAGACAATGGAACGTGTTACCCAGGAGATTTCATCGATTATGAGGAGCTA | |
| | AGAGAGCAATTGAGCTCAGTGTCATCATTTGAAAGGTTTGAGATATTCCCCA | |
| | AGACAAGTTCATGGCCCAATCATGACTCGAACAAAGGTGTAACGACAGCAT | |
| | GTCCTCATGCTGGAGCAAAAAGCTTCTACAAAAATTTAATATGGCTAGTTAA | |
| | AAAAGGAAATTCATACCCAAAGCTCAGCAAATCCTACATTAATGATAAAGGG | |
| | AAAGAAGTCCTCGTGCTATGGGGCATTCACCATCCACCTACTAGTGCTGAC | |
| | CAACAAAGTCTCTATCAGAATGCAGATGCATATGTTTTTGTGGGGACATCAA | |
| | GATACAGCAAGAAGTTCAAGCCGGAAATAGCAATAAGGCCCAAAGTGAGGG | |
| | ATCAAGAAGGGAGAATGAACTATTACTGGACACTAGTAGAGCCGGGAGACA | |
| | AAATAACATTCGAAGCAACTGGAAATCTAGTGGTACCGAGATATGCATTCGC | |
| | AATGGAAAGAAATGCTGGATCTGGTATTATCATTTCAGATACACCAGTCCAC | |
| | GATTGCAATACAACTTGTCAGACACCCAAGGGTGCTATAAACACCAGCCTC | |
| | CCATTTCAGAACATACATCCGATCACAATTGGAAAATGTCCAAAATATGTAAA | |
| | AAGCACAAAATTGAGACTGGCCACAGGATTGAGGAATGTCCCGTCCATTCA | |
| | ATCTAGAGGCCTATTTGGGGCCATTGCCGGTTTCATTGAAGGGGGGTGGAC | |
| | AGGGATGGTAGATGGATGGTACGGTTATCACCATCAAAATGAGCAGGGGTC | |
| | AGGATATGCAGCCGACCTGAAGAGCACACAGAATGCCATTGACGAGATTAC | |
| | TAACAAAGTAAACTCTGTTATTGAAAAAATGAATACACAGTTCACAGCAGTAG | |
| | GTAAAGAGTTCAACCACCTGGAAAAAAGAATAGAGAATTTAAATAAAAAAGT | |
| | TGATGATGGTTTCCTGGACATTTGGACTTACAATGCCGAACTGTTGGTTCTA | |
| | TTGGAAAATGAAAGAACTTTGGACTACCACGATTCAAAGGTGAAGAACTTAT | |
| | ATGAAAAGGTAAGAAGCCAGTTAAAAAACAATGCCAAGGAAATTGGAAACG | |
| | GCTGCTTTGAATTTTACCACAAATGTGATAACACGTGCATGGAAAGTGTCAA | |
| | AAATGGGACTTATGACTACCCAAAATACTCAGAGGAAGCAAAATTAAACAGA | |
| | GAAGAAATAGATGGGGTAAAGCTGGAATCAACAAGGATTTACCAGATTTTGG | |
| | CGATCTATTCAACCGTCGCCAGTTCATTGGTACTGGTAGTCTCCCTGGGGG | |
| | CAATCAGTTTCTGGATGTGCTCTAATGGGTCTCTACAGTGTAGAATATGTAT | |
| | TTAAGGAAGCGGAGCCACGAACTTCTCTCTGTTAAAGCAAGCAGGAGATGT | |
| | TGAAGAAAACCCCGGGCCTAGCAAAAGCAGGGGATAATTCTATTAACCATG | |
| | AAGACTATCATTGCTTTGAGCTACATTCTATGTCTGGTTTTCGCTCAAAAACT | |
| | TCCTGGAAATGACAATAGCACTGCAACGCTGTGCCTTGGGCACCATGCAGT | |
| | ACCAAACGGAACGATAGTGAAAACAATCACGAATGACCAAATTGAAGTTACT | |
| | AATGCTACTGAGCTGGTTCAGAATTCCTCAATAGGTGAAATATGCGACAGTC | |
| | CTCATCAGATCCTTGATGGAGAAAACTGCACACTAATAGATGCTCTATTGGG | |
| | AGACCCTCAGTGTGATGGCTTTCAAAATAAGAAATGGGACCTTTTTGTTGAA | |
| | CGAAGCAAAGCCTACAGCAACTGTTACCCTTATGATGTGCCGGATTATGCCT | |
| | CCCTTAGGTCACTAGTTGCCTCATCCGGCACACTGGAGTTTAACAATGAAAG | |
| | CTTCAATTGGGCTGGAGTCACTCAAAACGGAACAAGTTCTGCTTGCATAAG | |
| | GGGATCTAATAGTAGTTTCTTTAGTAGATTAAATTGGTTGACCCACTTAAACT | |
| | TCAAGTACCCAGCATTGAACGTGACTATGCCAAACAATGAACAATTTGACAA | |
| | ATTGTACATTTGGGGGGTTCACCACCCGGGTACGGACAAGGACCAAATCTT | |
| | CCTGTATGCTCAATCATCAGGAAGAATCACAGTATCTACCAAAAGAAGCCAA | |
| | CAAGCTGTAATCCCGAATATCGGATCTAGACCCAGAATAAGGAATATCCCTA | |
| | GCAGAATAAGCATCTATTGGACAATAGTAAAACCGGGAGACATACTTTTGAT | |
| | TAACAGCACAGGGAATCTAATTGCTCCTAGGGGTTACTTCAAAATACGAAGT | |
| | GGGAAAAGCTCAATAATGAGATCAGATGCACCCATTGGCAAATGCAAGTCT | |
| | GAATGCATCACTCCAAATGGAAGCATTCCCAATGACAAACCATTCCAAAATG | |
| | TAAACAGGATCACATACGGGGCCTGTCCCAGATATGTTAAGCAAAGCACTC | |
| | TGAAATTGGCAACAGGAATGCGAAATGTACCAGAGAAACAAACTAGAGGCA | |
| | TATTTGGCGCAATAGCGGGTTTCATAGAAAATGGTTGGGAGGGAATGGTGG | |
| | ATGGTTGGTACGGTTTCAGGCATCAAAATTCTGAGGGAAGAGGACAAGCAG | |
| | CAGATCTCAAAAGCACTCAAGCAGCAATCGATCAAATCAATGGGAAGCTGA | |
| | ATCGATTGATCGGGAAAACCAACGAGAAATTCCATCAGATTGAGAAAGAATT | |
| | CTCAGAAGTAGAAGGGAGAATTCAGGACCTTGAGAAATATGTTGAGGACAC | |
| | AAAAATAGATCTCTGGTCATACAACGCAGAGCTTCTTGTTGCCCTGGAAAAC | |
| | CAACATACAATTGATCTAACTGACTCAGAAATGAACAAACTGTTTGAAAAAAC | |
| | AAAGAAGCAACTGAGGGAAAATGCTGAGGATATGGGCAATGGTTGTTTCAA | |
| | AATATACCACAAATGTGACAATGCCTGCATAGGATCAATCAGAAATGGAACT | |
| | TATGACCACGATGTATACAGGGATGAAGCATTAAACAACCGGTTCCAGATCA | |
| | AGGGAGTTGAGCTGAAGTCAGGGTACAAAGATTGGATCCTATGGATTTCCT | |
| | TTGCCATATCATGTTTTTTGCTTTGTGTTGCTTTGTTGGGGTTCATCATGTGG | |
| | GCCTGCCAAAAGGGCAACATTAGGTGCAACATTTGCATTTGAGTGCATTAAT | |
| | TAAAAACACCCTTGTTTCTACTGGAAGCGGAGCCACGAACTTCTCTCTGTTA | |
| | AAGCAAGCAGGAGATGTTGAAGAAAACCCCGGGCCTATTTTCTAATATCCAC | |
| | AAAATGAAGGCAATAATTGTACTACTCATGGTAGTAACATCCAATGCAGATC | |
| | GAATCTGCACTGGGATAACATCGTCAAACTCACCACATGTCGTCAAAACTGC | |
| | TACTCAAGGGGAGGTCAACGTGACCGGTGTAATACCACTGACAACAACACC | |
| | CACCAAATCTCATTTTGCAAATCTCAAAGGAACAGAAACCAGGGGGAAACTA | |
| | TGCCCAAAATGCCTCAACTGCACAGATCTGGATGTAGCCTTGGGCAGACCA | |
| | AAATGCACAGGGAAAATACCCTCTGCAAGGGTTTCAATACTCCATGAAGTCA | |
| | GACCTGTTACATCTGGGTGCTTTCCTATAATGCACGATAGAACAAAAATTAG | |
| | ACAGCTGCCTAACCTTCTCCGAGGATACGAACATGTCAGGTTATCAACTCAC | |
| | AACGTTATCAATGCAAAAGATGCACCAGGAAGACCCTACGAAATTGGAACCT | |
| | CAGGGTCTTGCCCTAACATTACCAATGGAAACGGATTCTTCGCAACAATGG | |
| | CTTGGGCCGTCCCAAAAAACAAAACAGCAACAAATCCATTAACAATAGAAGT | |
| | ACCATACATTTGTACAGAAGGAGAAGACCAAATTACCGTTTGGGGGTTCCAC | |
| | TCTGACAACGAGACCCAAATGGCAAAGCTCTATGGGGACTCAAAGCCCCAG | |
| | AAGTTCACCTCATCTGCCAACGGAGTGACCACACATTACGTTTCACAGATTG | |
| | GTGGCTTCCCAAATCAAACAGAAGACGGAGGACTACCACAAAGTGGCAGAA | |
| | TTGTTGTTGATTACATGGTGCAGAAATCTGGAAAAACAGGAACAATTACCTA | |
| | TCAAAGAGGTATTTTATTGCCTCAAAAAGTGTGGTGCGCAAGTGGCAGGAG | |
| | CAAGGTAATAAAAGGATCCTTGCCCTTAATTGGAGAAGCAGATTGCCTCCAT | |
| | GAAAAATACGGTGGATTAAACAAAAGCAAGCCTTACTACACAGGGGAACAT | |
| | GCAAAGGCCATAGGAAATTGCCCAATATGGGTGAAAACACCCTTGAAGCTG | |
| | GCCAATGGAACCAAATATAGACCCCCTGCAAAACTATTAAAGGAAAGAGGTT | |
| | TCTTCGGAGCCATTGCTGGTTTCTTAGAGGGAGGATGGGAAGGAATGATTG | |
| | CAGGTTGGCACGGATACACATCCCATGGGGCACATGGAGTAGCGGTGGCA | |
| | GCTGACCTTAAGAGCACTCAAGAGGCCATAAACAAGATAACAAAAAATCTCA | |
| | ACTCTTTGAGTGAGCTGGAAGTAAAGAATCTTCAAAGACTAAGCGGTGCCAT | |
| | GGATGAACTCCACAACGAAATACTAGAACTAGATGAGAAAGTGGATGATCTC | |
| | AGAGCTGACACAATAAGCTCACAAATAGAACTCGCAGTCCTGCTTTCCAATG | |
| | AAGGAATAATAAACAGTGAAGATGAACATCTCTTGGCGCTTGAAAGAAAGCT | |
| | GAAGAAAATGCTGGGCCCCTCTGCTGTAGAGATAGGGAATGGATGCTTTGA | |
| | AACCAAACACAAGTGCAACCAGACCTGCCTCGACAGAATAGCTGCTGGTAC | |
| | CTTTGATGCAGGAGAATTTTCTCTCCCCACCTTTGATTCACTGAATATTACTG | |
| | CTGCATCTTTAAATGACGACGGATTGGACAATCATACTATACTGCTTTACTAC | |
| | TCAACTGCTGCCTCCAGTTTGGCTGTAACACTGATGATAGCTATCTTTGTTG | |
| | TTTATATGGTCTCCAGAGACAATGTTTCTTGCTCCATTTGTCTATAAGGAAAG | |
| | TTAAGCCCTGTATTTTCCTTTATTGTAGTGCTTGTTTGCTTGTTGTCATTACA | |
| | AAGAAACGTTATTGAAAAATGGAAGCGGAGCCACGAACTTCTCTCTGTTAAA | |
| | GCAAGCAGGAGATGTTGAAGAAAACCCCGGGCCTTTCGGAGCTATTGCTGG | |
| | TTTCTTGGAAGGAGGATGGGAAGGAATGATTGCAGGTTGGCACGGATACAC | |
| | ATCTCATGGAGCACATGGAGTAGCAGTGGCAGCAGACCTTAAGAGTACCCA | |
| | TGATGA | |
| Pan-CoV- | ATGTTCGTGTTCCTGGTGCTGCTGCCCCTGGTGAGCAGCCAGTGCGTGAAC | 44 |
| Flu | CTGATCACCAGGACCCAGAGCTACACCAACAGCTTCACCAGGGGCGTGTAC | |
| Vaccine | TACCCCGACAAGGTGTTCAGGAGCAGCGTGCTGCACAGCACCCAGGACCT | |
| #24 CoV- | GTTCCTGCCCTTCTTCAGCAACGTGACCTGGTTCCACGCCATCCACGTGAG | |
| Omicron | CGGCACCAACGGCACCAAGAGGTTCGACAACCCCGTGCTGCCCTTCAACG | |
| BA.2 Spike | ACGGCGTGTACTTCGCCAGCACCGAGAAGAGCAACATCATCAGGGGCTGG | |
| (2 proline | ATCTTCGGCACCACCCTGGACAGCAAGACCCAGAGCCTGCTGATCGTGAAC | |
| subst.)- | AACGCCACCAACGTGGTGATCAAGGTGTGCGAGTTCCAGTTCTGCAACGAC | |
| Influenza | CCCTTCCTGGACGTGTACTACCACAAGAACAACAAGAGCTGGATGGAGAGC | |
| (HA) | GAGTTCAGGGTGTACAGCAGCGCCAACAACTGCACCTTCGAGTACGTGAGC | |
| | CAGCCCTTCCTGATGGACCTGGAGGGCAAGCAGGGCAACTTCAAGAACCT | |
| | GAGGGAGTTCGTGTTCAAGAACATCGACGGCTACTTCAAGATCTACAGCAA | |
| | GCACACCCCCATCAACCTGGGCAGGGACCTGCCCCAGGGCTTCAGCGCCC | |
| | TGGAGCCCCTGGTGGACCTGCCCATCGGCATCAACATCACCAGGTTCCAGA | |
| | CCCTGCTGGCCCTGCACAGGAGCTACCTGACCCCCGGCGACAGCAGCAGC | |
| | GGCTGGACCGCCGGCGCCGCCGCCTACTACGTGGGCTACCTGCAGCCCA | |
| | GGACCTTCCTGCTGAAGTACAACGAGAACGGCACCATCACCGACGCCGTG | |
| | GACTGCGCCCTGGACCCCCTGAGCGAGACCAAGTGCACCCTGAAGAGCTT | |
| | CACCGTGGAGAAGGGCATCTACCAGACCAGCAACTTCAGGGTGCAGCCCA | |
| | CCGAGAGCATCGTGAGGTTCCCCAACATCACCAACCTGTGCCCCTTCGACG | |
| | AGGTGTTCAACGCCACCAGGTTCGCCAGCGTGTACGCCTGGAACAGGAAG | |
| | AGGATCAGCAACTGCGTGGCCGACTACAGCGTGCTGTACAACTTCGCCCCC | |
| | TTCTTCGCCTTCAAGTGCTACGGCGTGAGCCCCACCAAGCTGAACGACCTG | |
| | TGCTTCACCAACGTGTACGCCGACAGCTTCGTGATCAGGGGCAACGAGGT | |
| | GAGCCAGATCGCCCCCGGCCAGACCGGCAACATCGCCGACTACAACTACA | |
| | AGCTGCCCGACGACTTCACCGGCTGCGTGATCGCCTGGAACAGCAACAAG | |
| | CTGGACAGCAAGGTGGGCGGCAACTACAACTACCTGTACAGGCTGTTCAG | |
| | GAAGAGCAACCTGAAGCCCTTCGAGAGGGACATCAGCACCGAGATCTACCA | |
| | GGCCGGCAACAAGCCCTGCAACGGCGTGGCCGGCTTCAACTGCTACTTCC | |
| | CCCTGAGGAGCTACGGCTTCAGGCCCACCTACGGCGTGGGCCACCAGCCC | |
| | TACAGGGTGGTGGTGCTGAGCTTCGAGCTGCTGCACGCCCCCGCCACCGT | |
| | GTGCGGCCCCAAGAAGAGCACCAACCTGGTGAAGAACAAGTGCGTGAACT | |
| | TCAACTTCAACGGCCTGACCGGCACCGGCGTGCTGACCGAGAGCAACAAG | |
| | AAGTTCCTGCCCTTCCAGCAGTTCGGCAGGGACATCGCCGACACCACCGA | |
| | CGCCGTGAGGGACCCCCAGACCCTGGAGATCCTGGACATCACCCCCTGCA | |
| | GCTTCGGCGGCGTGAGCGTGATCACCCCCGGCACCAACACCAGCAACCAG | |
| | GTGGCCGTGCTGTACCAGGGCGTGAACTGCACCGAGGTGCCCGTGGCCAT | |
| | CCACGCCGACCAGCTGACCCCCACCTGGAGGGTGTACAGCACCGGCAGCA | |
| | ACGTGTTCCAGACCAGGGCCGGCTGCCTGATCGGCGCCGAGTACGTGAAC | |
| | AACAGCTACGAGTGCGACATCCCCATCGGCGCCGGCATCTGCGCCAGCTA | |
| | CCAGACCCAGACCAAGAGCCACCAGCAGGCCCAGAGCGTGGCCAGCCAGA | |
| | GCATCATCGCCTACACCATGAGCCTGGGCGCCGAGAACAGCGTGGCCTAC | |
| | AGCAACAACAGCATCGCCATCCCCACCAACTTCACCATCAGCGTGACCACC | |
| | GAGATCCTGCCCGTGAGCATGACCAAGACCAGCGTGGACTGCACCATGTA | |
| | CATCTGCGGCGACAGCACCGAGTGCAGCAACCTGCTGCTGCAGTACGGCA | |
| | GCTTCTGCACCCAGCTGAAGAGGGCCCTGACCGGCATCGCCGTGGAGCAG | |
| | GACAAGAACACCCAGGAGGTGTTCGCCCAGGTGAAGCAGATCTACAAGAC | |
| | CCCCCCCATCAAGTACTTCGGCGGCTTCAACTTCAGCCAGATCCTGCCCGA | |
| | CCCCAGCAAGCCCAGCAAGAGGAGCTTCATCGAGGACCTGCTGTTCAACAA | |
| | GGTGACCCTGGCCGACGCCGGCTTCATCAAGCAGTACGGCGACTGCCTGG | |
| | GCGACATCGCCGCCAGGGACCTGATCTGCGCCCAGAAGTTCAACGGCCTG | |
| | ACCGTGCTGCCCCCCCTGCTGACCGACGAGATGATCGCCCAGTACACCAG | |
| | CGCCCTGCTGGCCGGCACCATCACCAGCGGCTGGACCTTCGGCGCCGGC | |
| | GCCGCCCTGCAGATCCCCTTCGCCATGCAGATGGCCTACAGGTTCAACGG | |
| | CATCGGCGTGACCCAGAACGTGCTGTACGAGAACCAGAAGCTGATCGCCA | |
| | ACCAGTTCAACAGCGCCATCGGCAAGATCCAGGACAGCCTGAGCAGCACC | |
| | GCCAGCGCCCTGGGCAAGCTGCAGGACGTGGTGAACCACAACGCCCAGG | |
| | CCCTGAACACCCTGGTGAAGCAGCTGAGCAGCAAGTTCGGCGCCATCAGC | |
| | AGCGTGCTGAACGACATCCTGAGCAGGCTGGACCCCCCCGAGGCCGAGGT | |
| | GCAGATCGACAGGCTGATCACCGGCAGGCTGCAGAGCCTGCAGACCTACG | |
| | TGACCCAGCAGCTGATCAGGGCCGCCGAGATCAGGGCCAGCGCCAACCTG | |
| | GCCGCCACCAAGATGAGCGAGTGCGTGCTGGGCCAGAGCAAGAGGGTGG | |
| | ACTTCTGCGGCAAGGGCTACCACCTGATGAGCTTCCCCCAGAGCGCCCCC | |
| | CACGGCGTGGTGTTCCTGCACGTGACCTACGTGCCCGCCCAGGAGAAGAA | |
| | CTTCACCACCGCCCCCGCCATCTGCCACGACGGCAAGGCCCACTTCCCCA | |
| | GGGAGGGCGTGTTCGTGAGCAACGGCACCCACTGGTTCGTGACCCAGAGG | |
| | AACTTCTACGAGCCCCAGATCATCACCACCGACAACACCTTCGTGAGCGGC | |
| | AACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAA | |
| | CCACACCAGCCCCGACGTGGACCTGGGCGACATCAGCGGCATCAACGCCA | |
| | GCGTGGTGAACATCCAGAAGGAGATCGACAGGCTGAACGAGGTGGCCAAG | |
| | AACCTGAACGAGAGCCTGATCGACCTGCAGGAGCTGGGCAAGTACGAGCA | |
| | GTACATCAAGTGGCCCTGGTACATCTGGCTGGGCTTCATCGCCGGCCTGAT | |
| | CGCCATCGTGATGGTGACCATCATGCTGTGCTGCATGACCAGCTGCTGCAG | |
| | CTGCCTGAAGGGCTGCTGCAGCTGCGGCAGCTGCTGCAAGTTCGACGAGG | |
| | ACGACAGCGAGCCCGTGCTGAAGGGCGTGAAGCTGCACTACACCGGAAGC | |
| | GGAGCCACGAACTTCTCTCTGTTAAAGCAAGCAGGAGATGTTGAAGAAAAC | |
| | CCCGGGCCTATGAAGGCAATACTAGTAGTTCTGCTATATACATTTGCAACCG | |
| | CAAATGCAGACACATTATGTATAGGTTATCATGCGAACAACTCAACAGACAC | |
| | TGTAGACACAGTACTAGAAAAGAATGTAACAGTAACACACTCTGTTAACCTT | |
| | CTAGAAGACAAGCATAACGGGAAACTATGCAAACTAAGAGGGGTAGCCCCA | |
| | TTGCATTTGGGTAAATGTAACATTGCTGGCTGGATCCTGGGAAATCCAGAGT | |
| | GTGAATCACTCTCCACAGCAAGCTCATGGTCCTACATTGTGGAAACATCTAG | |
| | TTCAGACAATGGAACGTGTTACCCAGGAGATTTCATCGATTATGAGGAGCTA | |
| | AGAGAGCAATTGAGCTCAGTGTCATCATTTGAAAGGTTTGAGATATTCCCCA | |
| | AGACAAGTTCATGGCCCAATCATGACTCGAACAAAGGTGTAACGACAGCAT | |
| | GTCCTCATGCTGGAGCAAAAAGCTTCTACAAAAATTTAATATGGCTAGTTAA | |
| | AAAAGGAAATTCATACCCAAAGCTCAGCAAATCCTACATTAATGATAAAGGG | |
| | AAAGAAGTCCTCGTGCTATGGGGCATTCACCATCCACCTACTAGTGCTGAC | |
| | CAACAAAGTCTCTATCAGAATGCAGATGCATATGTTTTTGTGGGGACATCAA | |
| | GATACAGCAAGAAGTTCAAGCCGGAAATAGCAATAAGGCCCAAAGTGAGGG | |
| | ATCAAGAAGGGAGAATGAACTATTACTGGACACTAGTAGAGCCGGGAGACA | |
| | AAATAACATTCGAAGCAACTGGAAATCTAGTGGTACCGAGATATGCATTCGC | |
| | AATGGAAAGAAATGCTGGATCTGGTATTATCATTTCAGATACACCAGTCCAC | |
| | GATTGCAATACAACTTGTCAGACACCCAAGGGTGCTATAAACACCAGCCTC | |
| | CCATTTCAGAACATACATCCGATCACAATTGGAAAATGTCCAAAATATGTAAA | |
| | AAGCACAAAATTGAGACTGGCCACAGGATTGAGGAATGTCCCGTCCATTCA | |
| | ATCTAGAGGCCTATTTGGGGCCATTGCCGGTTTCATTGAAGGGGGGTGGAC | |
| | AGGGATGGTAGATGGATGGTACGGTTATCACCATCAAAATGAGCAGGGGTC | |
| | AGGATATGCAGCCGACCTGAAGAGCACACAGAATGCCATTGACGAGATTAC | |
| | TAACAAAGTAAACTCTGTTATTGAAAAAATGAATACACAGTTCACAGCAGTAG | |
| | GTAAAGAGTTCAACCACCTGGAAAAAAGAATAGAGAATTTAAATAAAAAAGT | |
| | TGATGATGGTTTCCTGGACATTTGGACTTACAATGCCGAACTGTTGGTTCTA | |
| | TTGGAAAATGAAAGAACTTTGGACTACCACGATTCAAAGGTGAAGAACTTAT | |
| | ATGAAAAGGTAAGAAGCCAGTTAAAAAACAATGCCAAGGAAATTGGAAACG | |
| | GCTGCTTTGAATTTTACCACAAATGTGATAACACGTGCATGGAAAGTGTCAA | |
| | AAATGGGACTTATGACTACCCAAAATACTCAGAGGAAGCAAAATTAAACAGA | |
| | GAAGAAATAGATGGGGTAAAGCTGGAATCAACAAGGATTTACCAGATTTTGG | |
| | CGATCTATTCAACCGTCGCCAGTTCATTGGTACTGGTAGTCTCCCTGGGGG | |
| | CAATCAGTTTCTGGATGTGCTCTAATGGGTCTCTACAGTGTAGAATATGTAT | |
| | TTAAGGAAGCGGAGCCACGAACTTCTCTCTGTTAAAGCAAGCAGGAGATGT | |
| | TGAAGAAAACCCCGGGCCTAGCAAAAGCAGGGGATAATTCTATTAACCATG | |
| | AAGACTATCATTGCTTTGAGCTACATTCTATGTCTGGTTTTCGCTCAAAAACT | |
| | TCCTGGAAATGACAATAGCACTGCAACGCTGTGCCTTGGGCACCATGCAGT | |
| | ACCAAACGGAACGATAGTGAAAACAATCACGAATGACCAAATTGAAGTTACT | |
| | AATGCTACTGAGCTGGTTCAGAATTCCTCAATAGGTGAAATATGCGACAGTC | |
| | CTCATCAGATCCTTGATGGAGAAAACTGCACACTAATAGATGCTCTATTGGG | |
| | AGACCCTCAGTGTGATGGCTTTCAAAATAAGAAATGGGACCTTTTTGTTGAA | |
| | CGAAGCAAAGCCTACAGCAACTGTTACCCTTATGATGTGCCGGATTATGCCT | |
| | CCCTTAGGTCACTAGTTGCCTCATCCGGCACACTGGAGTTTAACAATGAAAG | |
| | CTTCAATTGGGCTGGAGTCACTCAAAACGGAACAAGTTCTGCTTGCATAAG | |
| | GGGATCTAATAGTAGTTTCTTTAGTAGATTAAATTGGTTGACCCACTTAAACT | |
| | TCAAGTACCCAGCATTGAACGTGACTATGCCAAACAATGAACAATTTGACAA | |
| | ATTGTACATTTGGGGGGTTCACCACCCGGGTACGGACAAGGACCAAATCTT | |
| | CCTGTATGCTCAATCATCAGGAAGAATCACAGTATCTACCAAAAGAAGCCAA | |
| | CAAGCTGTAATCCCGAATATCGGATCTAGACCCAGAATAAGGAATATCCCTA | |
| | GCAGAATAAGCATCTATTGGACAATAGTAAAACCGGGAGACATACTTTTGAT | |
| | TAACAGCACAGGGAATCTAATTGCTCCTAGGGGTTACTTCAAAATACGAAGT | |
| | GGGAAAAGCTCAATAATGAGATCAGATGCACCCATTGGCAAATGCAAGTCT | |
| | GAATGCATCACTCCAAATGGAAGCATTCCCAATGACAAACCATTCCAAAATG | |
| | TAAACAGGATCACATACGGGGCCTGTCCCAGATATGTTAAGCAAAGCACTC | |
| | TGAAATTGGCAACAGGAATGCGAAATGTACCAGAGAAACAAACTAGAGGCA | |
| | TATTTGGCGCAATAGCGGGTTTCATAGAAAATGGTTGGGAGGGAATGGTGG | |
| | ATGGTTGGTACGGTTTCAGGCATCAAAATTCTGAGGGAAGAGGACAAGCAG | |
| | CAGATCTCAAAAGCACTCAAGCAGCAATCGATCAAATCAATGGGAAGCTGA | |
| | ATCGATTGATCGGGAAAACCAACGAGAAATTCCATCAGATTGAGAAAGAATT | |
| | CTCAGAAGTAGAAGGGAGAATTCAGGACCTTGAGAAATATGTTGAGGACAC | |
| | AAAAATAGATCTCTGGTCATACAACGCAGAGCTTCTTGTTGCCCTGGAAAAC | |
| | CAACATACAATTGATCTAACTGACTCAGAAATGAACAAACTGTTTGAAAAAAC | |
| | AAAGAAGCAACTGAGGGAAAATGCTGAGGATATGGGCAATGGTTGTTTCAA | |
| | AATATACCACAAATGTGACAATGCCTGCATAGGATCAATCAGAAATGGAACT | |
| | TATGACCACGATGTATACAGGGATGAAGCATTAAACAACCGGTTCCAGATCA | |
| | AGGGAGTTGAGCTGAAGTCAGGGTACAAAGATTGGATCCTATGGATTTCCT | |
| | TTGCCATATCATGTTTTTTGCTTTGTGTTGCTTTGTTGGGGTTCATCATGTGG | |
| | GCCTGCCAAAAGGGCAACATTAGGTGCAACATTTGCATTTGAGTGCATTAAT | |
| | TAAAAACACCCTTGTTTCTACTGGAAGCGGAGCCACGAACTTCTCTCTGTTA | |
| | AAGCAAGCAGGAGATGTTGAAGAAAACCCCGGGCCTATTTTCTAATATCCAC | |
| | AAAATGAAGGCAATAATTGTACTACTCATGGTAGTAACATCCAATGCAGATC | |
| | GAATCTGCACTGGGATAACATCGTCAAACTCACCACATGTCGTCAAAACTGC | |
| | TACTCAAGGGGAGGTCAACGTGACCGGTGTAATACCACTGACAACAACACC | |
| | CACCAAATCTCATTTTGCAAATCTCAAAGGAACAGAAACCAGGGGGAAACTA | |
| | TGCCCAAAATGCCTCAACTGCACAGATCTGGATGTAGCCTTGGGCAGACCA | |
| | AAATGCACAGGGAAAATACCCTCTGCAAGGGTTTCAATACTCCATGAAGTCA | |
| | GACCTGTTACATCTGGGTGCTTTCCTATAATGCACGATAGAACAAAAATTAG | |
| | ACAGCTGCCTAACCTTCTCCGAGGATACGAACATGTCAGGTTATCAACTCAC | |
| | AACGTTATCAATGCAAAAGATGCACCAGGAAGACCCTACGAAATTGGAACCT | |
| | CAGGGTCTTGCCCTAACATTACCAATGGAAACGGATTCTTCGCAACAATGG | |
| | CTTGGGCCGTCCCAAAAAACAAAACAGCAACAAATCCATTAACAATAGAAGT | |
| | ACCATACATTTGTACAGAAGGAGAAGACCAAATTACCGTTTGGGGGTTCCAC | |
| | TCTGACAACGAGACCCAAATGGCAAAGCTCTATGGGGACTCAAAGCCCCAG | |
| | AAGTTCACCTCATCTGCCAACGGAGTGACCACACATTACGTTTCACAGATTG | |
| | GTGGCTTCCCAAATCAAACAGAAGACGGAGGACTACCACAAAGTGGCAGAA | |
| | TTGTTGTTGATTACATGGTGCAGAAATCTGGAAAAACAGGAACAATTACCTA | |
| | TCAAAGAGGTATTTTATTGCCTCAAAAAGTGTGGTGCGCAAGTGGCAGGAG | |
| | CAAGGTAATAAAAGGATCCTTGCCCTTAATTGGAGAAGCAGATTGCCTCCAT | |
| | GAAAAATACGGTGGATTAAACAAAAGCAAGCCTTACTACACAGGGGAACAT | |
| | GCAAAGGCCATAGGAAATTGCCCAATATGGGTGAAAACACCCTTGAAGCTG | |
| | GCCAATGGAACCAAATATAGACCCCCTGCAAAACTATTAAAGGAAAGAGGTT | |
| | TCTTCGGAGCCATTGCTGGTTTCTTAGAGGGAGGATGGGAAGGAATGATTG | |
| | CAGGTTGGCACGGATACACATCCCATGGGGCACATGGAGTAGCGGTGGCA | |
| | GCTGACCTTAAGAGCACTCAAGAGGCCATAAACAAGATAACAAAAAATCTCA | |
| | ACTCTTTGAGTGAGCTGGAAGTAAAGAATCTTCAAAGACTAAGCGGTGCCAT | |
| | GGATGAACTCCACAACGAAATACTAGAACTAGATGAGAAAGTGGATGATCTC | |
| | AGAGCTGACACAATAAGCTCACAAATAGAACTCGCAGTCCTGCTTTCCAATG | |
| | AAGGAATAATAAACAGTGAAGATGAACATCTCTTGGCGCTTGAAAGAAAGCT | |
| | GAAGAAAATGCTGGGCCCCTCTGCTGTAGAGATAGGGAATGGATGCTTTGA | |
| | AACCAAACACAAGTGCAACCAGACCTGCCTCGACAGAATAGCTGCTGGTAC | |
| | CTTTGATGCAGGAGAATTTTCTCTCCCCACCTTTGATTCACTGAATATTACTG | |
| | CTGCATCTTTAAATGACGACGGATTGGACAATCATACTATACTGCTTTACTAC | |
| | TCAACTGCTGCCTCCAGTTTGGCTGTAACACTGATGATAGCTATCTTTGTTG | |
| | TTTATATGGTCTCCAGAGACAATGTTTCTTGCTCCATTTGTCTATAAGGAAAG | |
| | TTAAGCCCTGTATTTTCCTTTATTGTAGTGCTTGTTTGCTTGTTGTCATTACA | |
| | AAGAAACGTTATTGAAAAATGGAAGCGGAGCCACGAACTTCTCTCTGTTAAA | |
| | GCAAGCAGGAGATGTTGAAGAAAACCCCGGGCCTTTCGGAGCTATTGCTGG | |
| | TTTCTTGGAAGGAGGATGGGAAGGAATGATTGCAGGTTGGCACGGATACAC | |
| | ATCTCATGGAGCACATGGAGTAGCAGTGGCAGCAGACCTTAAGAGTACCCA | |
| | TGATGA | |
| Pan-CoV- | ATGTTCGTGTTCCTGGTGCTGCTGCCCCTGGTGAGCAGCCAGTGCGTGAAC | 45 |
| Flu | CTGACCACCAGGACCCAGCTGCCCCCCGCCTACACCAACAGCTTCACCAG | |
| Vaccine | GGGCGTGTACTACCCCGACAAGGTGTTCAGGAGCAGCGTGCTGCACAGCA | |
| #25 CoV- | CCCAGGACCTGTTCCTGCCCTTCTTCAGCAACGTGACCTGGTTCCACGCCA | |
| Wuhan | TCCACGTGAGCGGCACCAACGGCACCAAGAGGTTCGACAACCCCGTGCTG | |
| Spike | CCCTTCAACGACGGCGTGTACTTCGCCAGCACCGAGAAGAGCAACATCATC | |
| (HexaPro)- | AGGGGCTGGATCTTCGGCACCACCCTGGACAGCAAGACCCAGAGCCTGCT | |
| Influenza | GATCGTGAACAACGCCACCAACGTGGTGATCAAGGTGTGCGAGTTCCAGTT | |
| (HA) | CTGCAACGACCCCTTCCTGGGCGTGTACTACCACAAGAACAACAAGAGCTG | |
| | GATGGAGAGCGAGTTCAGGGTGTACAGCAGCGCCAACAACTGCACCTTCG | |
| | AGTACGTGAGCCAGCCCTTCCTGATGGACCTGGAGGGCAAGCAGGGCAAC | |
| | TTCAAGAACCTGAGGGAGTTCGTGTTCAAGAACATCGACGGCTACTTCAAG | |
| | ATCTACAGCAAGCACACCCCCATCAACCTGGTGAGGGACCTGCCCCAGGG | |
| | CTTCAGCGCCCTGGAGCCCCTGGTGGACCTGCCCATCGGCATCAACATCA | |
| | CCAGGTTCCAGACCCTGCTGGCCCTGCACAGGAGCTACCTGACCCCCGGC | |
| | GACAGCAGCAGCGGCTGGACCGCCGGCGCCGCCGCCTACTACGTGGGCT | |
| | ACCTGCAGCCCAGGACCTTCCTGCTGAAGTACAACGAGAACGGCACCATCA | |
| | CCGACGCCGTGGACTGCGCCCTGGACCCCCTGAGCGAGACCAAGTGCACC | |
| | CTGAAGAGCTTCACCGTGGAGAAGGGCATCTACCAGACCAGCAACTTCAGG | |
| | GTGCAGCCCACCGAGAGCATCGTGAGGTTCCCCAACATCACCAACCTGTGC | |
| | CCCTTCGGCGAGGTGTTCAACGCCACCAGGTTCGCCAGCGTGTACGCCTG | |
| | GAACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCAAG | |
| | CTGAACGACCTGTGCTTCACCAACGTGTACGCCGACAGCTTCGTGATCAGG | |
| | GGCGACGAGGTGAGGCAGATCGCCCCCGGCCAGACCGGCAAGATCGCCG | |
| | ACTACAACTACAAGCTGCCCGACGACTTCACCGGCTGCGTGATCGCCTGGA | |
| | ACAGCAACAACCTGGACAGCAAGGTGGGCGGCAACTACAACTACCTGTACA | |
| | GGCTGTTCAGGAAGAGCAACCTGAAGCCCTTCGAGAGGGACATCAGCACC | |
| | GAGATCTACCAGGCCGGCAGCACCCCCTGCAACGGCGTGGAGGGCTTCAA | |
| | CTGCTACTTCCCCCTGCAGAGCTACGGCTTCCAGCCCACCAACGGCGTGG | |
| | GCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAA | |
| | GTGCGTGAACTTCAACTTCAACGGCCTGACCGGCACCGGCGTGCTGACCG | |
| | AGAGCAACAAGAAGTTCCTGCCCTTCCAGCAGTTCGGCAGGGACATCGCC | |
| | GACACCACCGACGCCGTGAGGGACCCCCAGACCCTGGAGATCCTGGACAT | |
| | CACCCCCTGCAGCTTCGGCGGCGTGAGCGTGATCACCCCCGGCACCAACA | |
| | CCAGCAACCAGGTGGCCGTGCTGTACCAGGACGTGAACTGCACCGAGGTG | |
| | CCCGTGGCCATCCACGCCGACCAGCTGACCCCCACCTGGAGGGTGTACAG | |
| | CACCGGCAGCAACGTGTTCCAGACCAGGGCCGGCTGCCTGATCGGCGCCG | |
| | AGCACGTGAACAACAGCTACGAGTGCGACATCCCCATCGGCGCCGGCATC | |
| | TGCGCCAGCTACCAGACCCAGACCAACAGCCCCGGCAGCGCCAGCAGCGT | |
| | GGCCAGCCAGAGCATCATCGCCTACACCATGAGCCTGGGCGCCGAGAACA | |
| | GCGTGGCCTACAGCAACAACAGCATCGCCATCCCCACCAACTTCACCATCA | |
| | GCGTGACCACCGAGATCCTGCCCGTGAGCATGACCAAGACCAGCGTGGAC | |
| | TGCACCATGTACATCTGCGGCGACAGCACCGAGTGCAGCAACCTGCTGCT | |
| | GCAGTACGGCAGCTTCTGCACCCAGCTGAACAGGGCCCTGACCGGCATCG | |
| | CCGTGGAGCAGGACAAGAACACCCAGGAGGTGTTCGCCCAGGTGAAGCAG | |
| | ATCTACAAGACCCCCCCCATCAAGGACTTCGGCGGCTTCAACTTCAGCCAG | |
| | ATCCTGCCCGACCCCAGCAAGCCCAGCAAGAGGAGCCCCATCGAGGACCT | |
| | GCTGTTCAACAAGGTGACCCTGGCCGACGCCGGCTTCATCAAGCAGTACG | |
| | GCGACTGCCTGGGCGACATCGCCGCCAGGGACCTGATCTGCGCCCAGAAG | |
| | TTCAACGGCCTGACCGTGCTGCCCCCCCTGCTGACCGACGAGATGATCGC | |
| | CCAGTACACCAGCGCCCTGCTGGCCGGCACCATCACCAGCGGCTGGACCT | |
| | TCGGCGCCGGCCCCGCCCTGCAGATCCCCTTCCCCATGCAGATGGCCTAC | |
| | AGGTTCAACGGCATCGGCGTGACCCAGAACGTGCTGTACGAGAACCAGAA | |
| | GCTGATCGCCAACCAGTTCAACAGCGCCATCGGCAAGATCCAGGACAGCCT | |
| | GAGCAGCACCCCCAGCGCCCTGGGCAAGCTGCAGGACGTGGTGAACCAGA | |
| | ACGCCCAGGCCCTGAACACCCTGGTGAAGCAGCTGAGCAGCAACTTCGGC | |
| | GCCATCAGCAGCGTGCTGAACGACATCCTGAGCAGGCTGGACCCCCCCGA | |
| | GGCCGAGGTGCAGATCGACAGGCTGATCACCGGCAGGCTGCAGAGCCTGC | |
| | AGACCTACGTGACCCAGCAGCTGATCAGGGCCGCCGAGATCAGGGCCAGC | |
| | GCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGA | |
| | GCGCCCCCCACGGCGTGGTGTTCCTGCACGTGACCTACGTGCCCGCCCAG | |
| | GAGAAGAACTTCACCACCGCCCCCGCCATCTGCCACGACGGCAAGGCCCA | |
| | CTTCCCCAGGGAGGGCGTGTTCGTGAGCAACGGCACCCACTGGTTCGTGA | |
| | CCCAGAGGAACTTCTACGAGCCCCAGATCATCACCACCGACAACACCTTCG | |
| | TGAGCGGCAACTGCGACGTGGTGATCGGCATCGTGAACAACACCGTGTAC | |
| | GACCCCCTGCAGCCCGAGCTGGACAGCTTCAAGGAGGAGCTGGACAAGTA | |
| | CTTCAAGAACCACACCAGCCCCGACGTGGACCTGGGCGACATCAGCGGCA | |
| | TCAACGCCAGCGTGGTGAACATCCAGAAGGAGATCGACAGGCTGAACGAG | |
| | GTGGCCAAGAACCTGAACGAGAGCCTGATCGACCTGCAGGAGCTGGGCAA | |
| | GTACGAGCAGTACATCAAGTGGCCCTGGTACATCTGGCTGGGCTTCATCGC | |
| | CGGCCTGATCGCCATCGTGATGGTGACCATCATGCTGTGCTGCATGACCAG | |
| | CTGCTGCAGCTGCCTGAAGGGCTGCTGCAGCTGCGGCAGCTGCTGCAAGT | |
| | TCGACGAGGACGACAGCGAGCCCGTGCTGAAGGGCGTGAAGCTGCACTAC | |
| | ACCGGAAGCGGAGCCACGAACTTCTCTCTGTTAAAGCAAGCAGGAGATGTT | |
| | GAAGAAAACCCCGGGCCTATGAAGGCAATACTAGTAGTTCTGCTATATACAT | |
| | TTGCAACCGCAAATGCAGACACATTATGTATAGGTTATCATGCGAACAACTC | |
| | AACAGACACTGTAGACACAGTACTAGAAAAGAATGTAACAGTAACACACTCT | |
| | GTTAACCTTCTAGAAGACAAGCATAACGGGAAACTATGCAAACTAAGAGGG | |
| | GTAGCCCCATTGCATTTGGGTAAATGTAACATTGCTGGCTGGATCCTGGGA | |
| | AATCCAGAGTGTGAATCACTCTCCACAGCAAGCTCATGGTCCTACATTGTGG | |
| | AAACATCTAGTTCAGACAATGGAACGTGTTACCCAGGAGATTTCATCGATTA | |
| | TGAGGAGCTAAGAGAGCAATTGAGCTCAGTGTCATCATTTGAAAGGTTTGA | |
| | GATATTCCCCAAGACAAGTTCATGGCCCAATCATGACTCGAACAAAGGTGTA | |
| | ACGACAGCATGTCCTCATGCTGGAGCAAAAAGCTTCTACAAAAATTTAATAT | |
| | GGCTAGTTAAAAAAGGAAATTCATACCCAAAGCTCAGCAAATCCTACATTAA | |
| | TGATAAAGGGAAAGAAGTCCTCGTGCTATGGGGCATTCACCATCCACCTAC | |
| | TAGTGCTGACCAACAAAGTCTCTATCAGAATGCAGATGCATATGTTTTTGTG | |
| | GGGACATCAAGATACAGCAAGAAGTTCAAGCCGGAAATAGCAATAAGGCCC | |
| | AAAGTGAGGGATCAAGAAGGGAGAATGAACTATTACTGGACACTAGTAGAG | |
| | CCGGGAGACAAAATAACATTCGAAGCAACTGGAAATCTAGTGGTACCGAGA | |
| | TATGCATTCGCAATGGAAAGAAATGCTGGATCTGGTATTATCATTTCAGATA | |
| | CACCAGTCCACGATTGCAATACAACTTGTCAGACACCCAAGGGTGCTATAAA | |
| | CACCAGCCTCCCATTTCAGAACATACATCCGATCACAATTGGAAAATGTCCA | |
| | AAATATGTAAAAAGCACAAAATTGAGACTGGCCACAGGATTGAGGAATGTCC | |
| | CGTCCATTCAATCTAGAGGCCTATTTGGGGCCATTGCCGGTTTCATTGAAG | |
| | GGGGGTGGACAGGGATGGTAGATGGATGGTACGGTTATCACCATCAAAATG | |
| | AGCAGGGGTCAGGATATGCAGCCGACCTGAAGAGCACACAGAATGCCATT | |
| | GACGAGATTACTAACAAAGTAAACTCTGTTATTGAAAAAATGAATACACAGTT | |
| | CACAGCAGTAGGTAAAGAGTTCAACCACCTGGAAAAAAGAATAGAGAATTTA | |
| | AATAAAAAAGTTGATGATGGTTTCCTGGACATTTGGACTTACAATGCCGAAC | |
| | TGTTGGTTCTATTGGAAAATGAAAGAACTTTGGACTACCACGATTCAAAGGT | |
| | GAAGAACTTATATGAAAAGGTAAGAAGCCAGTTAAAAAACAATGCCAAGGAA | |
| | ATTGGAAACGGCTGCTTTGAATTTTACCACAAATGTGATAACACGTGCATGG | |
| | AAAGTGTCAAAAATGGGACTTATGACTACCCAAAATACTCAGAGGAAGCAAA | |
| | ATTAAACAGAGAAGAAATAGATGGGGTAAAGCTGGAATCAACAAGGATTTAC | |
| | CAGATTTTGGCGATCTATTCAACCGTCGCCAGTTCATTGGTACTGGTAGTCT | |
| | CCCTGGGGGCAATCAGTTTCTGGATGTGCTCTAATGGGTCTCTACAGTGTA | |
| | GAATATGTATTTAAGGAAGCGGAGCCACGAACTTCTCTCTGTTAAAGCAAGC | |
| | AGGAGATGTTGAAGAAAACCCCGGGCCTAGCAAAAGCAGGGGATAATTCTA | |
| | TTAACCATGAAGACTATCATTGCTTTGAGCTACATTCTATGTCTGGTTTTCGC | |
| | TCAAAAACTTCCTGGAAATGACAATAGCACTGCAACGCTGTGCCTTGGGCA | |
| | CCATGCAGTACCAAACGGAACGATAGTGAAAACAATCACGAATGACCAAATT | |
| | GAAGTTACTAATGCTACTGAGCTGGTTCAGAATTCCTCAATAGGTGAAATAT | |
| | GCGACAGTCCTCATCAGATCCTTGATGGAGAAAACTGCACACTAATAGATG | |
| | CTCTATTGGGAGACCCTCAGTGTGATGGCTTTCAAAATAAGAAATGGGACCT | |
| | TTTTGTTGAACGAAGCAAAGCCTACAGCAACTGTTACCCTTATGATGTGCCG | |
| | GATTATGCCTCCCTTAGGTCACTAGTTGCCTCATCCGGCACACTGGAGTTTA | |
| | ACAATGAAAGCTTCAATTGGGCTGGAGTCACTCAAAACGGAACAAGTTCTG | |
| | CTTGCATAAGGGGATCTAATAGTAGTTTCTTTAGTAGATTAAATTGGTTGACC | |
| | CACTTAAACTTCAAGTACCCAGCATTGAACGTGACTATGCCAAACAATGAAC | |
| | AATTTGACAAATTGTACATTTGGGGGGTTCACCACCCGGGTACGGACAAGG | |
| | ACCAAATCTTCCTGTATGCTCAATCATCAGGAAGAATCACAGTATCTACCAA | |
| | AAGAAGCCAACAAGCTGTAATCCCGAATATCGGATCTAGACCCAGAATAAG | |
| | GAATATCCCTAGCAGAATAAGCATCTATTGGACAATAGTAAAACCGGGAGAC | |
| | ATACTTTTGATTAACAGCACAGGGAATCTAATTGCTCCTAGGGGTTACTTCA | |
| | AAATACGAAGTGGGAAAAGCTCAATAATGAGATCAGATGCACCCATTGGCA | |
| | AATGCAAGTCTGAATGCATCACTCCAAATGGAAGCATTCCCAATGACAAACC | |
| | ATTCCAAAATGTAAACAGGATCACATACGGGGCCTGTCCCAGATATGTTAAG | |
| | CAAAGCACTCTGAAATTGGCAACAGGAATGCGAAATGTACCAGAGAAACAA | |
| | ACTAGAGGCATATTTGGCGCAATAGCGGGTTTCATAGAAAATGGTTGGGAG | |
| | GGAATGGTGGATGGTTGGTACGGTTTCAGGCATCAAAATTCTGAGGGAAGA | |
| | GGACAAGCAGCAGATCTCAAAAGCACTCAAGCAGCAATCGATCAAATCAAT | |
| | GGGAAGCTGAATCGATTGATCGGGAAAACCAACGAGAAATTCCATCAGATT | |
| | GAGAAAGAATTCTCAGAAGTAGAAGGGAGAATTCAGGACCTTGAGAAATAT | |
| | GTTGAGGACACAAAAATAGATCTCTGGTCATACAACGCAGAGCTTCTTGTTG | |
| | CCCTGGAAAACCAACATACAATTGATCTAACTGACTCAGAAATGAACAAACT | |
| | GTTTGAAAAAACAAAGAAGCAACTGAGGGAAAATGCTGAGGATATGGGCAA | |
| | TGGTTGTTTCAAAATATACCACAAATGTGACAATGCCTGCATAGGATCAATC | |
| | AGAAATGGAACTTATGACCACGATGTATACAGGGATGAAGCATTAAACAACC | |
| | GGTTCCAGATCAAGGGAGTTGAGCTGAAGTCAGGGTACAAAGATTGGATCC | |
| | TATGGATTTCCTTTGCCATATCATGTTTTTTGCTTTGTGTTGCTTTGTTGGGG | |
| | TTCATCATGTGGGCCTGCCAAAAGGGCAACATTAGGTGCAACATTTGCATTT | |
| | GAGTGCATTAATTAAAAACACCCTTGTTTCTACTGGAAGCGGAGCCACGAAC | |
| | TTCTCTCTGTTAAAGCAAGCAGGAGATGTTGAAGAAAACCCCGGGCCTATTT | |
| | TCTAATATCCACAAAATGAAGGCAATAATTGTACTACTCATGGTAGTAACATC | |
| | CAATGCAGATCGAATCTGCACTGGGATAACATCGTCAAACTCACCACATGTC | |
| | GTCAAAACTGCTACTCAAGGGGAGGTCAACGTGACCGGTGTAATACCACTG | |
| | ACAACAACACCCACCAAATCTCATTTTGCAAATCTCAAAGGAACAGAAACCA | |
| | GGGGGAAACTATGCCCAAAATGCCTCAACTGCACAGATCTGGATGTAGCCT | |
| | TGGGCAGACCAAAATGCACAGGGAAAATACCCTCTGCAAGGGTTTCAATAC | |
| | TCCATGAAGTCAGACCTGTTACATCTGGGTGCTTTCCTATAATGCACGATAG | |
| | AACAAAAATTAGACAGCTGCCTAACCTTCTCCGAGGATACGAACATGTCAGG | |
| | TTATCAACTCACAACGTTATCAATGCAAAAGATGCACCAGGAAGACCCTACG | |
| | AAATTGGAACCTCAGGGTCTTGCCCTAACATTACCAATGGAAACGGATTCTT | |
| | CGCAACAATGGCTTGGGCCGTCCCAAAAAACAAAACAGCAACAAATCCATT | |
| | AACAATAGAAGTACCATACATTTGTACAGAAGGAGAAGACCAAATTACCGTT | |
| | TGGGGGTTCCACTCTGACAACGAGACCCAAATGGCAAAGCTCTATGGGGAC | |
| | TCAAAGCCCCAGAAGTTCACCTCATCTGCCAACGGAGTGACCACACATTAC | |
| | GTTTCACAGATTGGTGGCTTCCCAAATCAAACAGAAGACGGAGGACTACCA | |
| | CAAAGTGGCAGAATTGTTGTTGATTACATGGTGCAGAAATCTGGAAAAACAG | |
| | GAACAATTACCTATCAAAGAGGTATTTTATTGCCTCAAAAAGTGTGGTGCGC | |
| | AAGTGGCAGGAGCAAGGTAATAAAAGGATCCTTGCCCTTAATTGGAGAAGC | |
| | AGATTGCCTCCATGAAAAATACGGTGGATTAAACAAAAGCAAGCCTTACTAC | |
| | ACAGGGGAACATGCAAAGGCCATAGGAAATTGCCCAATATGGGTGAAAACA | |
| | CCCTTGAAGCTGGCCAATGGAACCAAATATAGACCCCCTGCAAAACTATTAA | |
| | AGGAAAGAGGTTTCTTCGGAGCCATTGCTGGTTTCTTAGAGGGAGGATGGG | |
| | AAGGAATGATTGCAGGTTGGCACGGATACACATCCCATGGGGCACATGGAG | |
| | TAGCGGTGGCAGCTGACCTTAAGAGCACTCAAGAGGCCATAAACAAGATAA | |
| | CAAAAAATCTCAACTCTTTGAGTGAGCTGGAAGTAAAGAATCTTCAAAGACT | |
| | AAGCGGTGCCATGGATGAACTCCACAACGAAATACTAGAACTAGATGAGAA | |
| | AGTGGATGATCTCAGAGCTGACACAATAAGCTCACAAATAGAACTCGCAGT | |
| | CCTGCTTTCCAATGAAGGAATAATAAACAGTGAAGATGAACATCTCTTGGCG | |
| | CTTGAAAGAAAGCTGAAGAAAATGCTGGGCCCCTCTGCTGTAGAGATAGGG | |
| | AATGGATGCTTTGAAACCAAACACAAGTGCAACCAGACCTGCCTCGACAGA | |
| | ATAGCTGCTGGTACCTTTGATGCAGGAGAATTTTCTCTCCCCACCTTTGATT | |
| | CACTGAATATTACTGCTGCATCTTTAAATGACGACGGATTGGACAATCATAC | |
| | TATACTGCTTTACTACTCAACTGCTGCCTCCAGTTTGGCTGTAACACTGATG | |
| | ATAGCTATCTTTGTTGTTTATATGGTCTCCAGAGACAATGTTTCTTGCTCCAT | |
| | TTGTCTATAAGGAAAGTTAAGCCCTGTATTTTCCTTTATTGTAGTGCTTGTTT | |
| | GCTTGTTGTCATTACAAAGAAACGTTATTGAAAAATGGAAGCGGAGCCACGA | |
| | ACTTCTCTCTGTTAAAGCAAGCAGGAGATGTTGAAGAAAACCCCGGGCCTTT | |
| | CGGAGCTATTGCTGGTTTCTTGGAAGGAGGATGGGAAGGAATGATTGCAGG | |
| | TTGGCACGGATACACATCTCATGGAGCACATGGAGTAGCAGTGGCAGCAGA | |
| | CCTTAAGAGTACCCATGATGA | |
| Pan-CoV- | ATGTTCGTGTTCCTGGTGCTGCTGCCCCTGGTGAGCAGCCAGTGCGTGAAC | 46 |
| Flu | CTGACCACCAGGACCCAGCTGCCCCCCGCCTACACCAACAGCTTCACCAG | |
| Vaccine | GGGCGTGTACTACCCCGACAAGGTGTTCAGGAGCAGCGTGCTGCACAGCA | |
| #26 CoV- | CCCAGGACCTGTTCCTGCCCTTCTTCAGCAACGTGACCTGGTTCCACGCCA | |
| Wuhan | TCCACGTGAGCGGCACCAACGGCACCAAGAGGTTCGACAACCCCGTGCTG | |
| Spike | CCCTTCAACGACGGCGTGTACTTCGCCAGCACCGAGAAGAGCAACATCATC | |
| (2 proline | AGGGGCTGGATCTTCGGCACCACCCTGGACAGCAAGACCCAGAGCCTGCT | |
| subst.)- | GATCGTGAACAACGCCACCAACGTGGTGATCAAGGTGTGCGAGTTCCAGTT | |
| Influenza | CTGCAACGACCCCTTCCTGGGCGTGTACTACCACAAGAACAACAAGAGCTG | |
| (HA) | GATGGAGAGCGAGTTCAGGGTGTACAGCAGCGCCAACAACTGCACCTTCG | |
| | AGTACGTGAGCCAGCCCTTCCTGATGGACCTGGAGGGCAAGCAGGGCAAC | |
| | TTCAAGAACCTGAGGGAGTTCGTGTTCAAGAACATCGACGGCTACTTCAAG | |
| | ATCTACAGCAAGCACACCCCCATCAACCTGGTGAGGGACCTGCCCCAGGG | |
| | CTTCAGCGCCCTGGAGCCCCTGGTGGACCTGCCCATCGGCATCAACATCA | |
| | CCAGGTTCCAGACCCTGCTGGCCCTGCACAGGAGCTACCTGACCCCCGGC | |
| | GACAGCAGCAGCGGCTGGACCGCCGGCGCCGCCGCCTACTACGTGGGCT | |
| | ACCTGCAGCCCAGGACCTTCCTGCTGAAGTACAACGAGAACGGCACCATCA | |
| | CCGACGCCGTGGACTGCGCCCTGGACCCCCTGAGCGAGACCAAGTGCACC | |
| | CTGAAGAGCTTCACCGTGGAGAAGGGCATCTACCAGACCAGCAACTTCAGG | |
| | GTGCAGCCCACCGAGAGCATCGTGAGGTTCCCCAACATCACCAACCTGTGC | |
| | CCCTTCGGCGAGGTGTTCAACGCCACCAGGTTCGCCAGCGTGTACGCCTG | |
| | GAACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCAAG | |
| | CTGAACGACCTGTGCTTCACCAACGTGTACGCCGACAGCTTCGTGATCAGG | |
| | GGCGACGAGGTGAGGCAGATCGCCCCCGGCCAGACCGGCAAGATCGCCG | |
| | ACTACAACTACAAGCTGCCCGACGACTTCACCGGCTGCGTGATCGCCTGGA | |
| | ACAGCAACAACCTGGACAGCAAGGTGGGCGGCAACTACAACTACCTGTACA | |
| | GGCTGTTCAGGAAGAGCAACCTGAAGCCCTTCGAGAGGGACATCAGCACC | |
| | GAGATCTACCAGGCCGGCAGCACCCCCTGCAACGGCGTGGAGGGCTTCAA | |
| | CTGCTACTTCCCCCTGCAGAGCTACGGCTTCCAGCCCACCAACGGCGTGG | |
| | GCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAA | |
| | GTGCGTGAACTTCAACTTCAACGGCCTGACCGGCACCGGCGTGCTGACCG | |
| | AGAGCAACAAGAAGTTCCTGCCCTTCCAGCAGTTCGGCAGGGACATCGCC | |
| | GACACCACCGACGCCGTGAGGGACCCCCAGACCCTGGAGATCCTGGACAT | |
| | CACCCCCTGCAGCTTCGGCGGCGTGAGCGTGATCACCCCCGGCACCAACA | |
| | CCAGCAACCAGGTGGCCGTGCTGTACCAGGACGTGAACTGCACCGAGGTG | |
| | CCCGTGGCCATCCACGCCGACCAGCTGACCCCCACCTGGAGGGTGTACAG | |
| | CACCGGCAGCAACGTGTTCCAGACCAGGGCCGGCTGCCTGATCGGCGCCG | |
| | AGCACGTGAACAACAGCTACGAGTGCGACATCCCCATCGGCGCCGGCATC | |
| | TGCGCCAGCTACCAGACCCAGACCAACAGCCCCAGGAGGGCCAGGAGCGT | |
| | GGCCAGCCAGAGCATCATCGCCTACACCATGAGCCTGGGCGCCGAGAACA | |
| | GCGTGGCCTACAGCAACAACAGCATCGCCATCCCCACCAACTTCACCATCA | |
| | GCGTGACCACCGAGATCCTGCCCGTGAGCATGACCAAGACCAGCGTGGAC | |
| | TGCACCATGTACATCTGCGGCGACAGCACCGAGTGCAGCAACCTGCTGCT | |
| | GCAGTACGGCAGCTTCTGCACCCAGCTGAACAGGGCCCTGACCGGCATCG | |
| | CCGTGGAGCAGGACAAGAACACCCAGGAGGTGTTCGCCCAGGTGAAGCAG | |
| | ATCTACAAGACCCCCCCCATCAAGGACTTCGGCGGCTTCAACTTCAGCCAG | |
| | ATCCTGCCCGACCCCAGCAAGCCCAGCAAGAGGAGCTTCATCGAGGACCT | |
| | GCTGTTCAACAAGGTGACCCTGGCCGACGCCGGCTTCATCAAGCAGTACG | |
| | GCGACTGCCTGGGCGACATCGCCGCCAGGGACCTGATCTGCGCCCAGAAG | |
| | TTCAACGGCCTGACCGTGCTGCCCCCCCTGCTGACCGACGAGATGATCGC | |
| | CCAGTACACCAGCGCCCTGCTGGCCGGCACCATCACCAGCGGCTGGACCT | |
| | TCGGCGCCGGCGCCGCCCTGCAGATCCCCTTCGCCATGCAGATGGCCTAC | |
| | AGGTTCAACGGCATCGGCGTGACCCAGAACGTGCTGTACGAGAACCAGAA | |
| | GCTGATCGCCAACCAGTTCAACAGCGCCATCGGCAAGATCCAGGACAGCCT | |
| | GAGCAGCACCGCCAGCGCCCTGGGCAAGCTGCAGGACGTGGTGAACCAG | |
| | AACGCCCAGGCCCTGAACACCCTGGTGAAGCAGCTGAGCAGCAACTTCGG | |
| | CGCCATCAGCAGCGTGCTGAACGACATCCTGAGCAGGCTGGACCCCCCCG | |
| | AGGCCGAGGTGCAGATCGACAGGCTGATCACCGGCAGGCTGCAGAGCCTG | |
| | CAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAG | |
| | AGCGCCCCCCACGGCGTGGTGTTCCTGCACGTGACCTACGTGCCCGCCCA | |
| | GGAGAAGAACTTCACCACCGCCCCCGCCATCTGCCACGACGGCAAGGCCC | |
| | ACTTCCCCAGGGAGGGCGTGTTCGTGAGCAACGGCACCCACTGGTTCGTG | |
| | ACCCAGAGGAACTTCTACGAGCCCCAGATCATCACCACCGACAACACCTTC | |
| | GTGAGCGGCAACTGCGACGTGGTGATCGGCATCGTGAACAACACCGTGTA | |
| | CGACCCCCTGCAGCCCGAGCTGGACAGCTTCAAGGAGGAGCTGGACAAGT | |
| | ACTTCAAGAACCACACCAGCCCCGACGTGGACCTGGGCGACATCAGCGGC | |
| | ATCAACGCCAGCGTGGTGAACATCCAGAAGGAGATCGACAGGCTGAACGA | |
| | GGTGGCCAAGAACCTGAACGAGAGCCTGATCGACCTGCAGGAGCTGGGCA | |
| | AGTACGAGCAGTACATCAAGTGGCCCTGGTACATCTGGCTGGGCTTCATCG | |
| | CCGGCCTGATCGCCATCGTGATGGTGACCATCATGCTGTGCTGCATGACCA | |
| | GCTGCTGCAGCTGCCTGAAGGGCTGCTGCAGCTGCGGCAGCTGCTGCAAG | |
| | TTCGACGAGGACGACAGCGAGCCCGTGCTGAAGGGCGTGAAGCTGCACTA | |
| | CACCGGAAGCGGAGCCACGAACTTCTCTCTGTTAAAGCAAGCAGGAGATGT | |
| | TGAAGAAAACCCCGGGCCTATGAAGGCAATACTAGTAGTTCTGCTATATACA | |
| | TTTGCAACCGCAAATGCAGACACATTATGTATAGGTTATCATGCGAACAACT | |
| | CAACAGACACTGTAGACACAGTACTAGAAAAGAATGTAACAGTAACACACTC | |
| | TGTTAACCTTCTAGAAGACAAGCATAACGGGAAACTATGCAAACTAAGAGGG | |
| | GTAGCCCCATTGCATTTGGGTAAATGTAACATTGCTGGCTGGATCCTGGGA | |
| | AATCCAGAGTGTGAATCACTCTCCACAGCAAGCTCATGGTCCTACATTGTGG | |
| | AAACATCTAGTTCAGACAATGGAACGTGTTACCCAGGAGATTTCATCGATTA | |
| | TGAGGAGCTAAGAGAGCAATTGAGCTCAGTGTCATCATTTGAAAGGTTTGA | |
| | GATATTCCCCAAGACAAGTTCATGGCCCAATCATGACTCGAACAAAGGTGTA | |
| | ACGACAGCATGTCCTCATGCTGGAGCAAAAAGCTTCTACAAAAATTTAATAT | |
| | GGCTAGTTAAAAAAGGAAATTCATACCCAAAGCTCAGCAAATCCTACATTAA | |
| | TGATAAAGGGAAAGAAGTCCTCGTGCTATGGGGCATTCACCATCCACCTAC | |
| | TAGTGCTGACCAACAAAGTCTCTATCAGAATGCAGATGCATATGTTTTTGTG | |
| | GGGACATCAAGATACAGCAAGAAGTTCAAGCCGGAAATAGCAATAAGGCCC | |
| | AAAGTGAGGGATCAAGAAGGGAGAATGAACTATTACTGGACACTAGTAGAG | |
| | CCGGGAGACAAAATAACATTCGAAGCAACTGGAAATCTAGTGGTACCGAGA | |
| | TATGCATTCGCAATGGAAAGAAATGCTGGATCTGGTATTATCATTTCAGATA | |
| | CACCAGTCCACGATTGCAATACAACTTGTCAGACACCCAAGGGTGCTATAAA | |
| | CACCAGCCTCCCATTTCAGAACATACATCCGATCACAATTGGAAAATGTCCA | |
| | AAATATGTAAAAAGCACAAAATTGAGACTGGCCACAGGATTGAGGAATGTCC | |
| | CGTCCATTCAATCTAGAGGCCTATTTGGGGCCATTGCCGGTTTCATTGAAG | |
| | GGGGGTGGACAGGGATGGTAGATGGATGGTACGGTTATCACCATCAAAATG | |
| | AGCAGGGGTCAGGATATGCAGCCGACCTGAAGAGCACACAGAATGCCATT | |
| | GACGAGATTACTAACAAAGTAAACTCTGTTATTGAAAAAATGAATACACAGTT | |
| | CACAGCAGTAGGTAAAGAGTTCAACCACCTGGAAAAAAGAATAGAGAATTTA | |
| | AATAAAAAAGTTGATGATGGTTTCCTGGACATTTGGACTTACAATGCCGAAC | |
| | TGTTGGTTCTATTGGAAAATGAAAGAACTTTGGACTACCACGATTCAAAGGT | |
| | GAAGAACTTATATGAAAAGGTAAGAAGCCAGTTAAAAAACAATGCCAAGGAA | |
| | ATTGGAAACGGCTGCTTTGAATTTTACCACAAATGTGATAACACGTGCATGG | |
| | AAAGTGTCAAAAATGGGACTTATGACTACCCAAAATACTCAGAGGAAGCAAA | |
| | ATTAAACAGAGAAGAAATAGATGGGGTAAAGCTGGAATCAACAAGGATTTAC | |
| | CAGATTTTGGCGATCTATTCAACCGTCGCCAGTTCATTGGTACTGGTAGTCT | |
| | CCCTGGGGGCAATCAGTTTCTGGATGTGCTCTAATGGGTCTCTACAGTGTA | |
| | GAATATGTATTTAAGGAAGCGGAGCCACGAACTTCTCTCTGTTAAAGCAAGC | |
| | AGGAGATGTTGAAGAAAACCCCGGGCCTAGCAAAAGCAGGGGATAATTCTA | |
| | TTAACCATGAAGACTATCATTGCTTTGAGCTACATTCTATGTCTGGTTTTCGC | |
| | TCAAAAACTTCCTGGAAATGACAATAGCACTGCAACGCTGTGCCTTGGGCA | |
| | CCATGCAGTACCAAACGGAACGATAGTGAAAACAATCACGAATGACCAAATT | |
| | GAAGTTACTAATGCTACTGAGCTGGTTCAGAATTCCTCAATAGGTGAAATAT | |
| | GCGACAGTCCTCATCAGATCCTTGATGGAGAAAACTGCACACTAATAGATG | |
| | CTCTATTGGGAGACCCTCAGTGTGATGGCTTTCAAAATAAGAAATGGGACCT | |
| | TTTTGTTGAACGAAGCAAAGCCTACAGCAACTGTTACCCTTATGATGTGCCG | |
| | GATTATGCCTCCCTTAGGTCACTAGTTGCCTCATCCGGCACACTGGAGTTTA | |
| | ACAATGAAAGCTTCAATTGGGCTGGAGTCACTCAAAACGGAACAAGTTCTG | |
| | CTTGCATAAGGGGATCTAATAGTAGTTTCTTTAGTAGATTAAATTGGTTGACC | |
| | CACTTAAACTTCAAGTACCCAGCATTGAACGTGACTATGCCAAACAATGAAC | |
| | AATTTGACAAATTGTACATTTGGGGGGTTCACCACCCGGGTACGGACAAGG | |
| | ACCAAATCTTCCTGTATGCTCAATCATCAGGAAGAATCACAGTATCTACCAA | |
| | AAGAAGCCAACAAGCTGTAATCCCGAATATCGGATCTAGACCCAGAATAAG | |
| | GAATATCCCTAGCAGAATAAGCATCTATTGGACAATAGTAAAACCGGGAGAC | |
| | ATACTTTTGATTAACAGCACAGGGAATCTAATTGCTCCTAGGGGTTACTTCA | |
| | AAATACGAAGTGGGAAAAGCTCAATAATGAGATCAGATGCACCCATTGGCA | |
| | AATGCAAGTCTGAATGCATCACTCCAAATGGAAGCATTCCCAATGACAAACC | |
| | ATTCCAAAATGTAAACAGGATCACATACGGGGCCTGTCCCAGATATGTTAAG | |
| | CAAAGCACTCTGAAATTGGCAACAGGAATGCGAAATGTACCAGAGAAACAA | |
| | ACTAGAGGCATATTTGGCGCAATAGCGGGTTTCATAGAAAATGGTTGGGAG | |
| | GGAATGGTGGATGGTTGGTACGGTTTCAGGCATCAAAATTCTGAGGGAAGA | |
| | GGACAAGCAGCAGATCTCAAAAGCACTCAAGCAGCAATCGATCAAATCAAT | |
| | GGGAAGCTGAATCGATTGATCGGGAAAACCAACGAGAAATTCCATCAGATT | |
| | GAGAAAGAATTCTCAGAAGTAGAAGGGAGAATTCAGGACCTTGAGAAATAT | |
| | GTTGAGGACACAAAAATAGATCTCTGGTCATACAACGCAGAGCTTCTTGTTG | |
| | CCCTGGAAAACCAACATACAATTGATCTAACTGACTCAGAAATGAACAAACT | |
| | GTTTGAAAAAACAAAGAAGCAACTGAGGGAAAATGCTGAGGATATGGGCAA | |
| | TGGTTGTTTCAAAATATACCACAAATGTGACAATGCCTGCATAGGATCAATC | |
| | AGAAATGGAACTTATGACCACGATGTATACAGGGATGAAGCATTAAACAACC | |
| | GGTTCCAGATCAAGGGAGTTGAGCTGAAGTCAGGGTACAAAGATTGGATCC | |
| | TATGGATTTCCTTTGCCATATCATGTTTTTTGCTTTGTGTTGCTTTGTTGGGG | |
| | TTCATCATGTGGGCCTGCCAAAAGGGCAACATTAGGTGCAACATTTGCATTT | |
| | GAGTGCATTAATTAAAAACACCCTTGTTTCTACTGGAAGCGGAGCCACGAAC | |
| | TTCTCTCTGTTAAAGCAAGCAGGAGATGTTGAAGAAAACCCCGGGCCTATTT | |
| | TCTAATATCCACAAAATGAAGGCAATAATTGTACTACTCATGGTAGTAACATC | |
| | CAATGCAGATCGAATCTGCACTGGGATAACATCGTCAAACTCACCACATGTC | |
| | GTCAAAACTGCTACTCAAGGGGAGGTCAACGTGACCGGTGTAATACCACTG | |
| | ACAACAACACCCACCAAATCTCATTTTGCAAATCTCAAAGGAACAGAAACCA | |
| | GGGGGAAACTATGCCCAAAATGCCTCAACTGCACAGATCTGGATGTAGCCT | |
| | TGGGCAGACCAAAATGCACAGGGAAAATACCCTCTGCAAGGGTTTCAATAC | |
| | TCCATGAAGTCAGACCTGTTACATCTGGGTGCTTTCCTATAATGCACGATAG | |
| | AACAAAAATTAGACAGCTGCCTAACCTTCTCCGAGGATACGAACATGTCAGG | |
| | TTATCAACTCACAACGTTATCAATGCAAAAGATGCACCAGGAAGACCCTACG | |
| | AAATTGGAACCTCAGGGTCTTGCCCTAACATTACCAATGGAAACGGATTCTT | |
| | CGCAACAATGGCTTGGGCCGTCCCAAAAAACAAAACAGCAACAAATCCATT | |
| | AACAATAGAAGTACCATACATTTGTACAGAAGGAGAAGACCAAATTACCGTT | |
| | TGGGGGTTCCACTCTGACAACGAGACCCAAATGGCAAAGCTCTATGGGGAC | |
| | TCAAAGCCCCAGAAGTTCACCTCATCTGCCAACGGAGTGACCACACATTAC | |
| | GTTTCACAGATTGGTGGCTTCCCAAATCAAACAGAAGACGGAGGACTACCA | |
| | CAAAGTGGCAGAATTGTTGTTGATTACATGGTGCAGAAATCTGGAAAAACAG | |
| | GAACAATTACCTATCAAAGAGGTATTTTATTGCCTCAAAAAGTGTGGTGCGC | |
| | AAGTGGCAGGAGCAAGGTAATAAAAGGATCCTTGCCCTTAATTGGAGAAGC | |
| | AGATTGCCTCCATGAAAAATACGGTGGATTAAACAAAAGCAAGCCTTACTAC | |
| | ACAGGGGAACATGCAAAGGCCATAGGAAATTGCCCAATATGGGTGAAAACA | |
| | CCCTTGAAGCTGGCCAATGGAACCAAATATAGACCCCCTGCAAAACTATTAA | |
| | AGGAAAGAGGTTTCTTCGGAGCCATTGCTGGTTTCTTAGAGGGAGGATGGG | |
| | AAGGAATGATTGCAGGTTGGCACGGATACACATCCCATGGGGCACATGGAG | |
| | TAGCGGTGGCAGCTGACCTTAAGAGCACTCAAGAGGCCATAAACAAGATAA | |
| | CAAAAAATCTCAACTCTTTGAGTGAGCTGGAAGTAAAGAATCTTCAAAGACT | |
| | AAGCGGTGCCATGGATGAACTCCACAACGAAATACTAGAACTAGATGAGAA | |
| | AGTGGATGATCTCAGAGCTGACACAATAAGCTCACAAATAGAACTCGCAGT | |
| | CCTGCTTTCCAATGAAGGAATAATAAACAGTGAAGATGAACATCTCTTGGCG | |
| | CTTGAAAGAAAGCTGAAGAAAATGCTGGGCCCCTCTGCTGTAGAGATAGGG | |
| | AATGGATGCTTTGAAACCAAACACAAGTGCAACCAGACCTGCCTCGACAGA | |
| | ATAGCTGCTGGTACCTTTGATGCAGGAGAATTTTCTCTCCCCACCTTTGATT | |
| | CACTGAATATTACTGCTGCATCTTTAAATGACGACGGATTGGACAATCATAC | |
| | TATACTGCTTTACTACTCAACTGCTGCCTCCAGTTTGGCTGTAACACTGATG | |
| | ATAGCTATCTTTGTTGTTTATATGGTCTCCAGAGACAATGTTTCTTGCTCCAT | |
| | TTGTCTATAAGGAAAGTTAAGCCCTGTATTTTCCTTTATTGTAGTGCTTGTTT | |
| | GCTTGTTGTCATTACAAAGAAACGTTATTGAAAAATGGAAGCGGAGCCACGA | |
| | ACTTCTCTCTGTTAAAGCAAGCAGGAGATGTTGAAGAAAACCCCGGGCCTTT | |
| | CGGAGCTATTGCTGGTTTCTTGGAAGGAGGATGGGAAGGAATGATTGCAGG | |
| | TTGGCACGGATACACATCTCATGGAGCACATGGAGTAGCAGTGGCAGCAGA | |
| | CCTTAAGAGTACCCATGATGA | |
| Pan-CoV- | ATGTTCGTGTTCCTGGTGCTGCTGCCCCTGGTGAGCAGCCAGTGCGTGAAC | 47 |
| Flu | CTGATCACCAGGACCCAGAGCTACACCAACAGCTTCACCAGGGGCGTGTAC | |
| Vaccine | TACCCCGACAAGGTGTTCAGGAGCAGCGTGCTGCACAGCACCCAGGACCT | |
| #27 CoV- | GTTCCTGCCCTTCTTCAGCAACGTGACCTGGTTCCACGCCATCCACGTGAG | |
| Omicron | CGGCACCAACGGCACCAAGAGGTTCGACAACCCCGTGCTGCCCTTCAACG | |
| BA.2 Spike | ACGGCGTGTACTTCGCCAGCACCGAGAAGAGCAACATCATCAGGGGCTGG | |
| (HexaPro)- | ATCTTCGGCACCACCCTGGACAGCAAGACCCAGAGCCTGCTGATCGTGAAC | |
| Influenza | AACGCCACCAACGTGGTGATCAAGGTGTGCGAGTTCCAGTTCTGCAACGAC | |
| (HA) | CCCTTCCTGGACGTGTACTACCACAAGAACAACAAGAGCTGGATGGAGAGC | |
| | GAGTTCAGGGTGTACAGCAGCGCCAACAACTGCACCTTCGAGTACGTGAGC | |
| | CAGCCCTTCCTGATGGACCTGGAGGGCAAGCAGGGCAACTTCAAGAACCT | |
| | GAGGGAGTTCGTGTTCAAGAACATCGACGGCTACTTCAAGATCTACAGCAA | |
| | GCACACCCCCATCAACCTGGGCAGGGACCTGCCCCAGGGCTTCAGCGCCC | |
| | TGGAGCCCCTGGTGGACCTGCCCATCGGCATCAACATCACCAGGTTCCAGA | |
| | CCCTGCTGGCCCTGCACAGGAGCTACCTGACCCCCGGCGACAGCAGCAGC | |
| | GGCTGGACCGCCGGCGCCGCCGCCTACTACGTGGGCTACCTGCAGCCCA | |
| | GGACCTTCCTGCTGAAGTACAACGAGAACGGCACCATCACCGACGCCGTG | |
| | GACTGCGCCCTGGACCCCCTGAGCGAGACCAAGTGCACCCTGAAGAGCTT | |
| | CACCGTGGAGAAGGGCATCTACCAGACCAGCAACTTCAGGGTGCAGCCCA | |
| | CCGAGAGCATCGTGAGGTTCCCCAACATCACCAACCTGTGCCCCTTCGACG | |
| | AGGTGTTCAACGCCACCAGGTTCGCCAGCGTGTACGCCTGGAACAGGAAG | |
| | AGGATCAGCAACTGCGTGGCCGACTACAGCGTGCTGTACAACTTCGCCCCC | |
| | TTCTTCGCCTTCAAGTGCTACGGCGTGAGCCCCACCAAGCTGAACGACCTG | |
| | TGCTTCACCAACGTGTACGCCGACAGCTTCGTGATCAGGGGCAACGAGGT | |
| | GAGCCAGATCGCCCCCGGCCAGACCGGCAACATCGCCGACTACAACTACA | |
| | AGCTGCCCGACGACTTCACCGGCTGCGTGATCGCCTGGAACAGCAACAAG | |
| | CTGGACAGCAAGGTGGGCGGCAACTACAACTACCTGTACAGGCTGTTCAG | |
| | GAAGAGCAACCTGAAGCCCTTCGAGAGGGACATCAGCACCGAGATCTACCA | |
| | GGCCGGCAACAAGCCCTGCAACGGCGTGGCCGGCTTCAACTGCTACTTCC | |
| | CCCTGAGGAGCTACGGCTTCAGGCCCACCTACGGCGTGGGCCACCAGCCC | |
| | TACAGGGTGGTGGTGCTGAGCTTCGAGCTGCTGCACGCCCCCGCCACCGT | |
| | GTGCGGCCCCAAGAAGAGCACCAACCTGGTGAAGAACAAGTGCGTGAACT | |
| | TCAACTTCAACGGCCTGACCGGCACCGGCGTGCTGACCGAGAGCAACAAG | |
| | AAGTTCCTGCCCTTCCAGCAGTTCGGCAGGGACATCGCCGACACCACCGA | |
| | CGCCGTGAGGGACCCCCAGACCCTGGAGATCCTGGACATCACCCCCTGCA | |
| | GCTTCGGCGGCGTGAGCGTGATCACCCCCGGCACCAACACCAGCAACCAG | |
| | GTGGCCGTGCTGTACCAGGGCGTGAACTGCACCGAGGTGCCCGTGGCCAT | |
| | CCACGCCGACCAGCTGACCCCCACCTGGAGGGTGTACAGCACCGGCAGCA | |
| | ACGTGTTCCAGACCAGGGCCGGCTGCCTGATCGGCGCCGAGTACGTGAAC | |
| | AACAGCTACGAGTGCGACATCCCCATCGGCGCCGGCATCTGCGCCAGCTA | |
| | CCAGACCCAGACCAAGAGCCACCAGCAGGCCCAGAGCGTGGCCAGCCAGA | |
| | GCATCATCGCCTACACCATGAGCCTGGGCGCCGAGAACAGCGTGGCCTAC | |
| | AGCAACAACAGCATCGCCATCCCCACCAACTTCACCATCAGCGTGACCACC | |
| | GAGATCCTGCCCGTGAGCATGACCAAGACCAGCGTGGACTGCACCATGTA | |
| | CATCTGCGGCGACAGCACCGAGTGCAGCAACCTGCTGCTGCAGTACGGCA | |
| | GCTTCTGCACCCAGCTGAAGAGGGCCCTGACCGGCATCGCCGTGGAGCAG | |
| | GACAAGAACACCCAGGAGGTGTTCGCCCAGGTGAAGCAGATCTACAAGAC | |
| | CCCCCCCATCAAGTACTTCGGCGGCTTCAACTTCAGCCAGATCCTGCCCGA | |
| | CCCCAGCAAGCCCAGCAAGAGGAGCCCCATCGAGGACCTGCTGTTCAACA | |
| | AGGTGACCCTGGCCGACGCCGGCTTCATCAAGCAGTACGGCGACTGCCTG | |
| | GGCGACATCGCCGCCAGGGACCTGATCTGCGCCCAGAAGTTCAACGGCCT | |
| | GACCGTGCTGCCCCCCCTGCTGACCGACGAGATGATCGCCCAGTACACCA | |
| | GCGCCCTGCTGGCCGGCACCATCACCAGCGGCTGGACCTTCGGCGCCGG | |
| | CCCCGCCCTGCAGATCCCCTTCCCCATGCAGATGGCCTACAGGTTCAACGG | |
| | CATCGGCGTGACCCAGAACGTGCTGTACGAGAACCAGAAGCTGATCGCCA | |
| | ACCAGTTCAACAGCGCCATCGGCAAGATCCAGGACAGCCTGAGCAGCACC | |
| | CCCAGCGCCCTGGGCAAGCTGCAGGACGTGGTGAACCACAACGCCCAGGC | |
| | CCTGAACACCCTGGTGAAGCAGCTGAGCAGCAAGTTCGGCGCCATCAGCA | |
| | GCGTGCTGAACGACATCCTGAGCAGGCTGGACCCCCCCGAGGCCGAGGTG | |
| | CAGATCGACAGGCTGATCACCGGCAGGCTGCAGAGCCTGCAGACCTACGT | |
| | GACCCAGCAGCTGATCAGGGCCGCCGAGATCAGGGCCAGCGCCAACCTG | |
| | GCCGCCACCAAGATGAGCGAGTGCGTGCTGGGCCAGAGCAAGAGGGTGG | |
| | ACTTCTGCGGCAAGGGCTACCACCTGATGAGCTTCCCCCAGAGCGCCCCC | |
| | CACGGCGTGGTGTTCCTGCACGTGACCTACGTGCCCGCCCAGGAGAAGAA | |
| | CTTCACCACCGCCCCCGCCATCTGCCACGACGGCAAGGCCCACTTCCCCA | |
| | GGGAGGGCGTGTTCGTGAGCAACGGCACCCACTGGTTCGTGACCCAGAGG | |
| | AACTTCTACGAGCCCCAGATCATCACCACCGACAACACCTTCGTGAGCGGC | |
| | AACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAA | |
| | CCACACCAGCCCCGACGTGGACCTGGGCGACATCAGCGGCATCAACGCCA | |
| | GCGTGGTGAACATCCAGAAGGAGATCGACAGGCTGAACGAGGTGGCCAAG | |
| | AACCTGAACGAGAGCCTGATCGACCTGCAGGAGCTGGGCAAGTACGAGCA | |
| | GTACATCAAGTGGCCCTGGTACATCTGGCTGGGCTTCATCGCCGGCCTGAT | |
| | CGCCATCGTGATGGTGACCATCATGCTGTGCTGCATGACCAGCTGCTGCAG | |
| | CTGCCTGAAGGGCTGCTGCAGCTGCGGCAGCTGCTGCAAGTTCGACGAGG | |
| | ACGACAGCGAGCCCGTGCTGAAGGGCGTGAAGCTGCACTACACCGGAAGC | |
| | GGAGCCACGAACTTCTCTCTGTTAAAGCAAGCAGGAGATGTTGAAGAAAAC | |
| | CCCGGGCCTATGAAGGCAATACTAGTAGTTCTGCTATATACATTTGCAACCG | |
| | CAAATGCAGACACATTATGTATAGGTTATCATGCGAACAACTCAACAGACAC | |
| | TGTAGACACAGTACTAGAAAAGAATGTAACAGTAACACACTCTGTTAACCTT | |
| | CTAGAAGACAAGCATAACGGGAAACTATGCAAACTAAGAGGGGTAGCCCCA | |
| | TTGCATTTGGGTAAATGTAACATTGCTGGCTGGATCCTGGGAAATCCAGAGT | |
| | GTGAATCACTCTCCACAGCAAGCTCATGGTCCTACATTGTGGAAACATCTAG | |
| | TTCAGACAATGGAACGTGTTACCCAGGAGATTTCATCGATTATGAGGAGCTA | |
| | AGAGAGCAATTGAGCTCAGTGTCATCATTTGAAAGGTTTGAGATATTCCCCA | |
| | AGACAAGTTCATGGCCCAATCATGACTCGAACAAAGGTGTAACGACAGCAT | |
| | GTCCTCATGCTGGAGCAAAAAGCTTCTACAAAAATTTAATATGGCTAGTTAA | |
| | AAAAGGAAATTCATACCCAAAGCTCAGCAAATCCTACATTAATGATAAAGGG | |
| | AAAGAAGTCCTCGTGCTATGGGGCATTCACCATCCACCTACTAGTGCTGAC | |
| | CAACAAAGTCTCTATCAGAATGCAGATGCATATGTTTTTGTGGGGACATCAA | |
| | GATACAGCAAGAAGTTCAAGCCGGAAATAGCAATAAGGCCCAAAGTGAGGG | |
| | ATCAAGAAGGGAGAATGAACTATTACTGGACACTAGTAGAGCCGGGAGACA | |
| | AAATAACATTCGAAGCAACTGGAAATCTAGTGGTACCGAGATATGCATTCGC | |
| | AATGGAAAGAAATGCTGGATCTGGTATTATCATTTCAGATACACCAGTCCAC | |
| | GATTGCAATACAACTTGTCAGACACCCAAGGGTGCTATAAACACCAGCCTC | |
| | CCATTTCAGAACATACATCCGATCACAATTGGAAAATGTCCAAAATATGTAAA | |
| | AAGCACAAAATTGAGACTGGCCACAGGATTGAGGAATGTCCCGTCCATTCA | |
| | ATCTAGAGGCCTATTTGGGGCCATTGCCGGTTTCATTGAAGGGGGGTGGAC | |
| | AGGGATGGTAGATGGATGGTACGGTTATCACCATCAAAATGAGCAGGGGTC | |
| | AGGATATGCAGCCGACCTGAAGAGCACACAGAATGCCATTGACGAGATTAC | |
| | TAACAAAGTAAACTCTGTTATTGAAAAAATGAATACACAGTTCACAGCAGTAG | |
| | GTAAAGAGTTCAACCACCTGGAAAAAAGAATAGAGAATTTAAATAAAAAAGT | |
| | TGATGATGGTTTCCTGGACATTTGGACTTACAATGCCGAACTGTTGGTTCTA | |
| | TTGGAAAATGAAAGAACTTTGGACTACCACGATTCAAAGGTGAAGAACTTAT | |
| | ATGAAAAGGTAAGAAGCCAGTTAAAAAACAATGCCAAGGAAATTGGAAACG | |
| | GCTGCTTTGAATTTTACCACAAATGTGATAACACGTGCATGGAAAGTGTCAA | |
| | AAATGGGACTTATGACTACCCAAAATACTCAGAGGAAGCAAAATTAAACAGA | |
| | GAAGAAATAGATGGGGTAAAGCTGGAATCAACAAGGATTTACCAGATTTTGG | |
| | CGATCTATTCAACCGTCGCCAGTTCATTGGTACTGGTAGTCTCCCTGGGGG | |
| | CAATCAGTTTCTGGATGTGCTCTAATGGGTCTCTACAGTGTAGAATATGTAT | |
| | TTAAGGAAGCGGAGCCACGAACTTCTCTCTGTTAAAGCAAGCAGGAGATGT | |
| | TGAAGAAAACCCCGGGCCTAGCAAAAGCAGGGGATAATTCTATTAACCATG | |
| | AAGACTATCATTGCTTTGAGCTACATTCTATGTCTGGTTTTCGCTCAAAAACT | |
| | TCCTGGAAATGACAATAGCACTGCAACGCTGTGCCTTGGGCACCATGCAGT | |
| | ACCAAACGGAACGATAGTGAAAACAATCACGAATGACCAAATTGAAGTTACT | |
| | AATGCTACTGAGCTGGTTCAGAATTCCTCAATAGGTGAAATATGCGACAGTC | |
| | CTCATCAGATCCTTGATGGAGAAAACTGCACACTAATAGATGCTCTATTGGG | |
| | AGACCCTCAGTGTGATGGCTTTCAAAATAAGAAATGGGACCTTTTTGTTGAA | |
| | CGAAGCAAAGCCTACAGCAACTGTTACCCTTATGATGTGCCGGATTATGCCT | |
| | CCCTTAGGTCACTAGTTGCCTCATCCGGCACACTGGAGTTTAACAATGAAAG | |
| | CTTCAATTGGGCTGGAGTCACTCAAAACGGAACAAGTTCTGCTTGCATAAG | |
| | GGGATCTAATAGTAGTTTCTTTAGTAGATTAAATTGGTTGACCCACTTAAACT | |
| | TCAAGTACCCAGCATTGAACGTGACTATGCCAAACAATGAACAATTTGACAA | |
| | ATTGTACATTTGGGGGGTTCACCACCCGGGTACGGACAAGGACCAAATCTT | |
| | CCTGTATGCTCAATCATCAGGAAGAATCACAGTATCTACCAAAAGAAGCCAA | |
| | CAAGCTGTAATCCCGAATATCGGATCTAGACCCAGAATAAGGAATATCCCTA | |
| | GCAGAATAAGCATCTATTGGACAATAGTAAAACCGGGAGACATACTTTTGAT | |
| | TAACAGCACAGGGAATCTAATTGCTCCTAGGGGTTACTTCAAAATACGAAGT | |
| | GGGAAAAGCTCAATAATGAGATCAGATGCACCCATTGGCAAATGCAAGTCT | |
| | GAATGCATCACTCCAAATGGAAGCATTCCCAATGACAAACCATTCCAAAATG | |
| | TAAACAGGATCACATACGGGGCCTGTCCCAGATATGTTAAGCAAAGCACTC | |
| | TGAAATTGGCAACAGGAATGCGAAATGTACCAGAGAAACAAACTAGAGGCA | |
| | TATTTGGCGCAATAGCGGGTTTCATAGAAAATGGTTGGGAGGGAATGGTGG | |
| | ATGGTTGGTACGGTTTCAGGCATCAAAATTCTGAGGGAAGAGGACAAGCAG | |
| | CAGATCTCAAAAGCACTCAAGCAGCAATCGATCAAATCAATGGGAAGCTGA | |
| | ATCGATTGATCGGGAAAACCAACGAGAAATTCCATCAGATTGAGAAAGAATT | |
| | CTCAGAAGTAGAAGGGAGAATTCAGGACCTTGAGAAATATGTTGAGGACAC | |
| | AAAAATAGATCTCTGGTCATACAACGCAGAGCTTCTTGTTGCCCTGGAAAAC | |
| | CAACATACAATTGATCTAACTGACTCAGAAATGAACAAACTGTTTGAAAAAAC | |
| | AAAGAAGCAACTGAGGGAAAATGCTGAGGATATGGGCAATGGTTGTTTCAA | |
| | AATATACCACAAATGTGACAATGCCTGCATAGGATCAATCAGAAATGGAACT | |
| | TATGACCACGATGTATACAGGGATGAAGCATTAAACAACCGGTTCCAGATCA | |
| | AGGGAGTTGAGCTGAAGTCAGGGTACAAAGATTGGATCCTATGGATTTCCT | |
| | TTGCCATATCATGTTTTTTGCTTTGTGTTGCTTTGTTGGGGTTCATCATGTGG | |
| | GCCTGCCAAAAGGGCAACATTAGGTGCAACATTTGCATTTGAGTGCATTAAT | |
| | TAAAAACACCCTTGTTTCTACTGGAAGCGGAGCCACGAACTTCTCTCTGTTA | |
| | AAGCAAGCAGGAGATGTTGAAGAAAACCCCGGGCCTATTTTCTAATATCCAC | |
| | AAAATGAAGGCAATAATTGTACTACTCATGGTAGTAACATCCAATGCAGATC | |
| | GAATCTGCACTGGGATAACATCGTCAAACTCACCACATGTCGTCAAAACTGC | |
| | TACTCAAGGGGAGGTCAACGTGACCGGTGTAATACCACTGACAACAACACC | |
| | CACCAAATCTCATTTTGCAAATCTCAAAGGAACAGAAACCAGGGGGAAACTA | |
| | TGCCCAAAATGCCTCAACTGCACAGATCTGGATGTAGCCTTGGGCAGACCA | |
| | AAATGCACAGGGAAAATACCCTCTGCAAGGGTTTCAATACTCCATGAAGTCA | |
| | GACCTGTTACATCTGGGTGCTTTCCTATAATGCACGATAGAACAAAAATTAG | |
| | ACAGCTGCCTAACCTTCTCCGAGGATACGAACATGTCAGGTTATCAACTCAC | |
| | AACGTTATCAATGCAAAAGATGCACCAGGAAGACCCTACGAAATTGGAACCT | |
| | CAGGGTCTTGCCCTAACATTACCAATGGAAACGGATTCTTCGCAACAATGG | |
| | CTTGGGCCGTCCCAAAAAACAAAACAGCAACAAATCCATTAACAATAGAAGT | |
| | ACCATACATTTGTACAGAAGGAGAAGACCAAATTACCGTTTGGGGGTTCCAC | |
| | TCTGACAACGAGACCCAAATGGCAAAGCTCTATGGGGACTCAAAGCCCCAG | |
| | AAGTTCACCTCATCTGCCAACGGAGTGACCACACATTACGTTTCACAGATTG | |
| | GTGGCTTCCCAAATCAAACAGAAGACGGAGGACTACCACAAAGTGGCAGAA | |
| | TTGTTGTTGATTACATGGTGCAGAAATCTGGAAAAACAGGAACAATTACCTA | |
| | TCAAAGAGGTATTTTATTGCCTCAAAAAGTGTGGTGCGCAAGTGGCAGGAG | |
| | CAAGGTAATAAAAGGATCCTTGCCCTTAATTGGAGAAGCAGATTGCCTCCAT | |
| | GAAAAATACGGTGGATTAAACAAAAGCAAGCCTTACTACACAGGGGAACAT | |
| | GCAAAGGCCATAGGAAATTGCCCAATATGGGTGAAAACACCCTTGAAGCTG | |
| | GCCAATGGAACCAAATATAGACCCCCTGCAAAACTATTAAAGGAAAGAGGTT | |
| | TCTTCGGAGCCATTGCTGGTTTCTTAGAGGGAGGATGGGAAGGAATGATTG | |
| | CAGGTTGGCACGGATACACATCCCATGGGGCACATGGAGTAGCGGTGGCA | |
| | GCTGACCTTAAGAGCACTCAAGAGGCCATAAACAAGATAACAAAAAATCTCA | |
| | ACTCTTTGAGTGAGCTGGAAGTAAAGAATCTTCAAAGACTAAGCGGTGCCAT | |
| | GGATGAACTCCACAACGAAATACTAGAACTAGATGAGAAAGTGGATGATCTC | |
| | AGAGCTGACACAATAAGCTCACAAATAGAACTCGCAGTCCTGCTTTCCAATG | |
| | AAGGAATAATAAACAGTGAAGATGAACATCTCTTGGCGCTTGAAAGAAAGCT | |
| | GAAGAAAATGCTGGGCCCCTCTGCTGTAGAGATAGGGAATGGATGCTTTGA | |
| | AACCAAACACAAGTGCAACCAGACCTGCCTCGACAGAATAGCTGCTGGTAC | |
| | CTTTGATGCAGGAGAATTTTCTCTCCCCACCTTTGATTCACTGAATATTACTG | |
| | CTGCATCTTTAAATGACGACGGATTGGACAATCATACTATACTGCTTTACTAC | |
| | TCAACTGCTGCCTCCAGTTTGGCTGTAACACTGATGATAGCTATCTTTGTTG | |
| | TTTATATGGTCTCCAGAGACAATGTTTCTTGCTCCATTTGTCTATAAGGAAAG | |
| | TTAAGCCCTGTATTTTCCTTTATTGTAGTGCTTGTTTGCTTGTTGTCATTACA | |
| | AAGAAACGTTATTGAAAAATTGATGA | |
| Pan-CoV- | ATGTTCGTGTTCCTGGTGCTGCTGCCCCTGGTGAGCAGCCAGTGCGTGAAC | 48 |
| Flu | CTGATCACCAGGACCCAGAGCTACACCAACAGCTTCACCAGGGGCGTGTAC | |
| Vaccine | TACCCCGACAAGGTGTTCAGGAGCAGCGTGCTGCACAGCACCCAGGACCT | |
| #28 CoV- | GTTCCTGCCCTTCTTCAGCAACGTGACCTGGTTCCACGCCATCCACGTGAG | |
| Omicron | CGGCACCAACGGCACCAAGAGGTTCGACAACCCCGTGCTGCCCTTCAACG | |
| BA.2 | ACGGCGTGTACTTCGCCAGCACCGAGAAGAGCAACATCATCAGGGGCTGG | |
| Spike | ATCTTCGGCACCACCCTGGACAGCAAGACCCAGAGCCTGCTGATCGTGAAC | |
| (2 proline | AACGCCACCAACGTGGTGATCAAGGTGTGCGAGTTCCAGTTCTGCAACGAC | |
| subst.)- | CCCTTCCTGGACGTGTACTACCACAAGAACAACAAGAGCTGGATGGAGAGC | |
| Influenza | GAGTTCAGGGTGTACAGCAGCGCCAACAACTGCACCTTCGAGTACGTGAGC | |
| (HA) | CAGCCCTTCCTGATGGACCTGGAGGGCAAGCAGGGCAACTTCAAGAACCT | |
| | GAGGGAGTTCGTGTTCAAGAACATCGACGGCTACTTCAAGATCTACAGCAA | |
| | GCACACCCCCATCAACCTGGGCAGGGACCTGCCCCAGGGCTTCAGCGCCC | |
| | TGGAGCCCCTGGTGGACCTGCCCATCGGCATCAACATCACCAGGTTCCAGA | |
| | CCCTGCTGGCCCTGCACAGGAGCTACCTGACCCCCGGCGACAGCAGCAGC | |
| | GGCTGGACCGCCGGCGCCGCCGCCTACTACGTGGGCTACCTGCAGCCCA | |
| | GGACCTTCCTGCTGAAGTACAACGAGAACGGCACCATCACCGACGCCGTG | |
| | GACTGCGCCCTGGACCCCCTGAGCGAGACCAAGTGCACCCTGAAGAGCTT | |
| | CACCGTGGAGAAGGGCATCTACCAGACCAGCAACTTCAGGGTGCAGCCCA | |
| | CCGAGAGCATCGTGAGGTTCCCCAACATCACCAACCTGTGCCCCTTCGACG | |
| | AGGTGTTCAACGCCACCAGGTTCGCCAGCGTGTACGCCTGGAACAGGAAG | |
| | AGGATCAGCAACTGCGTGGCCGACTACAGCGTGCTGTACAACTTCGCCCCC | |
| | TTCTTCGCCTTCAAGTGCTACGGCGTGAGCCCCACCAAGCTGAACGACCTG | |
| | TGCTTCACCAACGTGTACGCCGACAGCTTCGTGATCAGGGGCAACGAGGT | |
| | GAGCCAGATCGCCCCCGGCCAGACCGGCAACATCGCCGACTACAACTACA | |
| | AGCTGCCCGACGACTTCACCGGCTGCGTGATCGCCTGGAACAGCAACAAG | |
| | CTGGACAGCAAGGTGGGGGGCAACTACAACTACCTGTACAGGCTGTTCAG | |
| | GAAGAGCAACCTGAAGCCCTTCGAGAGGGACATCAGCACCGAGATCTACCA | |
| | GGCCGGCAACAAGCCCTGCAACGGCGTGGCCGGCTTCAACTGCTACTTCC | |
| | CCCTGAGGAGCTACGGCTTCAGGCCCACCTACGGCGTGGGCCACCAGCCC | |
| | TACAGGGTGGTGGTGCTGAGCTTCGAGCTGCTGCACGCCCCCGCCACCGT | |
| | GTGCGGCCCCAAGAAGAGCACCAACCTGGTGAAGAACAAGTGCGTGAACT | |
| | TCAACTTCAACGGCCTGACCGGCACCGGCGTGCTGACCGAGAGCAACAAG | |
| | AAGTTCCTGCCCTTCCAGCAGTTCGGCAGGGACATCGCCGACACCACCGA | |
| | CGCCGTGAGGGACCCCCAGACCCTGGAGATCCTGGACATCACCCCCTGCA | |
| | GCTTCGGCGGCGTGAGCGTGATCACCCCCGGCACCAACACCAGCAACCAG | |
| | GTGGCCGTGCTGTACCAGGGCGTGAACTGCACCGAGGTGCCCGTGGCCAT | |
| | CCACGCCGACCAGCTGACCCCCACCTGGAGGGTGTACAGCACCGGCAGCA | |
| | ACGTGTTCCAGACCAGGGCCGGCTGCCTGATCGGCGCCGAGTACGTGAAC | |
| | AACAGCTACGAGTGCGACATCCCCATCGGCGCCGGCATCTGCGCCAGCTA | |
| | CCAGACCCAGACCAAGAGCCACCAGCAGGCCCAGAGCGTGGCCAGCCAGA | |
| | GCATCATCGCCTACACCATGAGCCTGGGCGCCGAGAACAGCGTGGCCTAC | |
| | AGCAACAACAGCATCGCCATCCCCACCAACTTCACCATCAGCGTGACCACC | |
| | GAGATCCTGCCCGTGAGCATGACCAAGACCAGCGTGGACTGCACCATGTA | |
| | CATCTGCGGCGACAGCACCGAGTGCAGCAACCTGCTGCTGCAGTACGGCA | |
| | GCTTCTGCACCCAGCTGAAGAGGGCCCTGACCGGCATCGCCGTGGAGCAG | |
| | GACAAGAACACCCAGGAGGTGTTCGCCCAGGTGAAGCAGATCTACAAGAC | |
| | CCCCCCCATCAAGTACTTCGGCGGCTTCAACTTCAGCCAGATCCTGCCCGA | |
| | CCCCAGCAAGCCCAGCAAGAGGAGCTTCATCGAGGACCTGCTGTTCAACAA | |
| | GGTGACCCTGGCCGACGCCGGCTTCATCAAGCAGTACGGCGACTGCCTGG | |
| | GCGACATCGCCGCCAGGGACCTGATCTGCGCCCAGAAGTTCAACGGCCTG | |
| | ACCGTGCTGCCCCCCCTGCTGACCGACGAGATGATCGCCCAGTACACCAG | |
| | CGCCCTGCTGGCCGGCACCATCACCAGCGGCTGGACCTTCGGCGCCGGC | |
| | GCCGCCCTGCAGATCCCCTTCGCCATGCAGATGGCCTACAGGTTCAACGG | |
| | CATCGGCGTGACCCAGAACGTGCTGTACGAGAACCAGAAGCTGATCGCCA | |
| | ACCAGTTCAACAGCGCCATCGGCAAGATCCAGGACAGCCTGAGCAGCACC | |
| | GCCAGCGCCCTGGGCAAGCTGCAGGACGTGGTGAACCACAACGCCCAGG | |
| | CCCTGAACACCCTGGTGAAGCAGCTGAGCAGCAAGTTCGGCGCCATCAGC | |
| | AGCGTGCTGAACGACATCCTGAGCAGGCTGGACCCCCCCGAGGCCGAGGT | |
| | GCAGATCGACAGGCTGATCACCGGCAGGCTGCAGAGCCTGCAGACCTACG | |
| | TGACCCAGCAGCTGATCAGGGCCGCCGAGATCAGGGCCAGCGCCAACCTG | |
| | GCCGCCACCAAGATGAGCGAGTGCGTGCTGGGCCAGAGCAAGAGGGTGG | |
| | ACTTCTGCGGCAAGGGCTACCACCTGATGAGCTTCCCCCAGAGCGCCCCC | |
| | CACGGCGTGGTGTTCCTGCACGTGACCTACGTGCCCGCCCAGGAGAAGAA | |
| | CTTCACCACCGCCCCCGCCATCTGCCACGACGGCAAGGCCCACTTCCCCA | |
| | GGGAGGGCGTGTTCGTGAGCAACGGCACCCACTGGTTCGTGACCCAGAGG | |
| | AACTTCTACGAGCCCCAGATCATCACCACCGACAACACCTTCGTGAGCGGC | |
| | AACTGCGACGTGGTGATCGGCATCGTGAACAACACCGTGTACGACCCCCTG | |
| | CAGCCCGAGCTGGACAGCTTCAAGGAGGAGCTGGACAAGTACTTCAAGAA | |
| | CCACACCAGCCCCGACGTGGACCTGGGCGACATCAGCGGCATCAACGCCA | |
| | GCGTGGTGAACATCCAGAAGGAGATCGACAGGCTGAACGAGGTGGCCAAG | |
| | AACCTGAACGAGAGCCTGATCGACCTGCAGGAGCTGGGCAAGTACGAGCA | |
| | GTACATCAAGTGGCCCTGGTACATCTGGCTGGGCTTCATCGCCGGCCTGAT | |
| | CGCCATCGTGATGGTGACCATCATGCTGTGCTGCATGACCAGCTGCTGCAG | |
| | CTGCCTGAAGGGCTGCTGCAGCTGCGGCAGCTGCTGCAAGTTCGACGAGG | |
| | ACGACAGCGAGCCCGTGCTGAAGGGCGTGAAGCTGCACTACACCGGAAGC | |
| | GGAGCCACGAACTTCTCTCTGTTAAAGCAAGCAGGAGATGTTGAAGAAAAC | |
| | CCCGGGCCTATGAAGGCAATACTAGTAGTTCTGCTATATACATTTGCAACCG | |
| | CAAATGCAGACACATTATGTATAGGTTATCATGCGAACAACTCAACAGACAC | |
| | TGTAGACACAGTACTAGAAAAGAATGTAACAGTAACACACTCTGTTAACCTT | |
| | CTAGAAGACAAGCATAACGGGAAACTATGCAAACTAAGAGGGGTAGCCCCA | |
| | TTGCATTTGGGTAAATGTAACATTGCTGGCTGGATCCTGGGAAATCCAGAGT | |
| | GTGAATCACTCTCCACAGCAAGCTCATGGTCCTACATTGTGGAAACATCTAG | |
| | TTCAGACAATGGAACGTGTTACCCAGGAGATTTCATCGATTATGAGGAGCTA | |
| | AGAGAGCAATTGAGCTCAGTGTCATCATTTGAAAGGTTTGAGATATTCCCCA | |
| | AGACAAGTTCATGGCCCAATCATGACTCGAACAAAGGTGTAACGACAGCAT | |
| | GTCCTCATGCTGGAGCAAAAAGCTTCTACAAAAATTTAATATGGCTAGTTAA | |
| | AAAAGGAAATTCATACCCAAAGCTCAGCAAATCCTACATTAATGATAAAGGG | |
| | AAAGAAGTCCTCGTGCTATGGGGCATTCACCATCCACCTACTAGTGCTGAC | |
| | CAACAAAGTCTCTATCAGAATGCAGATGCATATGTTTTTGTGGGGACATCAA | |
| | GATACAGCAAGAAGTTCAAGCCGGAAATAGCAATAAGGCCCAAAGTGAGGG | |
| | ATCAAGAAGGGAGAATGAACTATTACTGGACACTAGTAGAGCCGGGAGACA | |
| | AAATAACATTCGAAGCAACTGGAAATCTAGTGGTACCGAGATATGCATTCGC | |
| | AATGGAAAGAAATGCTGGATCTGGTATTATCATTTCAGATACACCAGTCCAC | |
| | GATTGCAATACAACTTGTCAGACACCCAAGGGTGCTATAAACACCAGCCTC | |
| | CCATTTCAGAACATACATCCGATCACAATTGGAAAATGTCCAAAATATGTAAA | |
| | AAGCACAAAATTGAGACTGGCCACAGGATTGAGGAATGTCCCGTCCATTCA | |
| | ATCTAGAGGCCTATTTGGGGCCATTGCCGGTTTCATTGAAGGGGGGTGGAC | |
| | AGGGATGGTAGATGGATGGTACGGTTATCACCATCAAAATGAGCAGGGGTC | |
| | AGGATATGCAGCCGACCTGAAGAGCACACAGAATGCCATTGACGAGATTAC | |
| | TAACAAAGTAAACTCTGTTATTGAAAAAATGAATACACAGTTCACAGCAGTAG | |
| | GTAAAGAGTTCAACCACCTGGAAAAAAGAATAGAGAATTTAAATAAAAAAGT | |
| | TGATGATGGTTTCCTGGACATTTGGACTTACAATGCCGAACTGTTGGTTCTA | |
| | TTGGAAAATGAAAGAACTTTGGACTACCACGATTCAAAGGTGAAGAACTTAT | |
| | ATGAAAAGGTAAGAAGCCAGTTAAAAAACAATGCCAAGGAAATTGGAAACG | |
| | GCTGCTTTGAATTTTACCACAAATGTGATAACACGTGCATGGAAAGTGTCAA | |
| | AAATGGGACTTATGACTACCCAAAATACTCAGAGGAAGCAAAATTAAACAGA | |
| | GAAGAAATAGATGGGGTAAAGCTGGAATCAACAAGGATTTACCAGATTTTGG | |
| | CGATCTATTCAACCGTCGCCAGTTCATTGGTACTGGTAGTCTCCCTGGGGG | |
| | CAATCAGTTTCTGGATGTGCTCTAATGGGTCTCTACAGTGTAGAATATGTAT | |
| | TTAAGGAAGCGGAGCCACGAACTTCTCTCTGTTAAAGCAAGCAGGAGATGT | |
| | TGAAGAAAACCCCGGGCCTAGCAAAAGCAGGGGATAATTCTATTAACCATG | |
| | AAGACTATCATTGCTTTGAGCTACATTCTATGTCTGGTTTTCGCTCAAAAACT | |
| | TCCTGGAAATGACAATAGCACTGCAACGCTGTGCCTTGGGCACCATGCAGT | |
| | ACCAAACGGAACGATAGTGAAAACAATCACGAATGACCAAATTGAAGTTACT | |
| | AATGCTACTGAGCTGGTTCAGAATTCCTCAATAGGTGAAATATGCGACAGTC | |
| | CTCATCAGATCCTTGATGGAGAAAACTGCACACTAATAGATGCTCTATTGGG | |
| | AGACCCTCAGTGTGATGGCTTTCAAAATAAGAAATGGGACCTTTTTGTTGAA | |
| | CGAAGCAAAGCCTACAGCAACTGTTACCCTTATGATGTGCCGGATTATGCCT | |
| | CCCTTAGGTCACTAGTTGCCTCATCCGGCACACTGGAGTTTAACAATGAAAG | |
| | CTTCAATTGGGCTGGAGTCACTCAAAACGGAACAAGTTCTGCTTGCATAAG | |
| | GGGATCTAATAGTAGTTTCTTTAGTAGATTAAATTGGTTGACCCACTTAAACT | |
| | TCAAGTACCCAGCATTGAACGTGACTATGCCAAACAATGAACAATTTGACAA | |
| | ATTGTACATTTGGGGGGTTCACCACCCGGGTACGGACAAGGACCAAATCTT | |
| | CCTGTATGCTCAATCATCAGGAAGAATCACAGTATCTACCAAAAGAAGCCAA | |
| | CAAGCTGTAATCCCGAATATCGGATCTAGACCCAGAATAAGGAATATCCCTA | |
| | GCAGAATAAGCATCTATTGGACAATAGTAAAACCGGGAGACATACTTTTGAT | |
| | TAACAGCACAGGGAATCTAATTGCTCCTAGGGGTTACTTCAAAATACGAAGT | |
| | GGGAAAAGCTCAATAATGAGATCAGATGCACCCATTGGCAAATGCAAGTCT | |
| | GAATGCATCACTCCAAATGGAAGCATTCCCAATGACAAACCATTCCAAAATG | |
| | TAAACAGGATCACATACGGGGCCTGTCCCAGATATGTTAAGCAAAGCACTC | |
| | TGAAATTGGCAACAGGAATGCGAAATGTACCAGAGAAACAAACTAGAGGCA | |
| | TATTTGGCGCAATAGCGGGTTTCATAGAAAATGGTTGGGAGGGAATGGTGG | |
| | ATGGTTGGTACGGTTTCAGGCATCAAAATTCTGAGGGAAGAGGACAAGCAG | |
| | CAGATCTCAAAAGCACTCAAGCAGCAATCGATCAAATCAATGGGAAGCTGA | |
| | ATCGATTGATCGGGAAAACCAACGAGAAATTCCATCAGATTGAGAAAGAATT | |
| | CTCAGAAGTAGAAGGGAGAATTCAGGACCTTGAGAAATATGTTGAGGACAC | |
| | AAAAATAGATCTCTGGTCATACAACGCAGAGCTTCTTGTTGCCCTGGAAAAC | |
| | CAACATACAATTGATCTAACTGACTCAGAAATGAACAAACTGTTTGAAAAAAC | |
| | AAAGAAGCAACTGAGGGAAAATGCTGAGGATATGGGCAATGGTTGTTTCAA | |
| | AATATACCACAAATGTGACAATGCCTGCATAGGATCAATCAGAAATGGAACT | |
| | TATGACCACGATGTATACAGGGATGAAGCATTAAACAACCGGTTCCAGATCA | |
| | AGGGAGTTGAGCTGAAGTCAGGGTACAAAGATTGGATCCTATGGATTTCCT | |
| | TTGCCATATCATGTTTTTTGCTTTGTGTTGCTTTGTTGGGGTTCATCATGTGG | |
| | GCCTGCCAAAAGGGCAACATTAGGTGCAACATTTGCATTTGAGTGCATTAAT | |
| | TAAAAACACCCTTGTTTCTACTGGAAGCGGAGCCACGAACTTCTCTCTGTTA | |
| | AAGCAAGCAGGAGATGTTGAAGAAAACCCCGGGCCTATTTTCTAATATCCAC | |
| | AAAATGAAGGCAATAATTGTACTACTCATGGTAGTAACATCCAATGCAGATC | |
| | GAATCTGCACTGGGATAACATCGTCAAACTCACCACATGTCGTCAAAACTGC | |
| | TACTCAAGGGGAGGTCAACGTGACCGGTGTAATACCACTGACAACAACACC | |
| | CACCAAATCTCATTTTGCAAATCTCAAAGGAACAGAAACCAGGGGGAAACTA | |
| | TGCCCAAAATGCCTCAACTGCACAGATCTGGATGTAGCCTTGGGCAGACCA | |
| | AAATGCACAGGGAAAATACCCTCTGCAAGGGTTTCAATACTCCATGAAGTCA | |
| | GACCTGTTACATCTGGGTGCTTTCCTATAATGCACGATAGAACAAAAATTAG | |
| | ACAGCTGCCTAACCTTCTCCGAGGATACGAACATGTCAGGTTATCAACTCAC | |
| | AACGTTATCAATGCAAAAGATGCACCAGGAAGACCCTACGAAATTGGAACCT | |
| | CAGGGTCTTGCCCTAACATTACCAATGGAAACGGATTCTTCGCAACAATGG | |
| | CTTGGGCCGTCCCAAAAAACAAAACAGCAACAAATCCATTAACAATAGAAGT | |
| | ACCATACATTTGTACAGAAGGAGAAGACCAAATTACCGTTTGGGGGTTCCAC | |
| | TCTGACAACGAGACCCAAATGGCAAAGCTCTATGGGGACTCAAAGCCCCAG | |
| | AAGTTCACCTCATCTGCCAACGGAGTGACCACACATTACGTTTCACAGATTG | |
| | GTGGCTTCCCAAATCAAACAGAAGACGGAGGACTACCACAAAGTGGCAGAA | |
| | TTGTTGTTGATTACATGGTGCAGAAATCTGGAAAAACAGGAACAATTACCTA | |
| | TCAAAGAGGTATTTTATTGCCTCAAAAAGTGTGGTGCGCAAGTGGCAGGAG | |
| | CAAGGTAATAAAAGGATCCTTGCCCTTAATTGGAGAAGCAGATTGCCTCCAT | |
| | GAAAAATACGGTGGATTAAACAAAAGCAAGCCTTACTACACAGGGGAACAT | |
| | GCAAAGGCCATAGGAAATTGCCCAATATGGGTGAAAACACCCTTGAAGCTG | |
| | GCCAATGGAACCAAATATAGACCCCCTGCAAAACTATTAAAGGAAAGAGGTT | |
| | TCTTCGGAGCCATTGCTGGTTTCTTAGAGGGAGGATGGGAAGGAATGATTG | |
| | CAGGTTGGCACGGATACACATCCCATGGGGCACATGGAGTAGCGGTGGCA | |
| | GCTGACCTTAAGAGCACTCAAGAGGCCATAAACAAGATAACAAAAAATCTCA | |
| | ACTCTTTGAGTGAGCTGGAAGTAAAGAATCTTCAAAGACTAAGCGGTGCCAT | |
| | GGATGAACTCCACAACGAAATACTAGAACTAGATGAGAAAGTGGATGATCTC | |
| | AGAGCTGACACAATAAGCTCACAAATAGAACTCGCAGTCCTGCTTTCCAATG | |
| | AAGGAATAATAAACAGTGAAGATGAACATCTCTTGGCGCTTGAAAGAAAGCT | |
| | GAAGAAAATGCTGGGCCCCTCTGCTGTAGAGATAGGGAATGGATGCTTTGA | |
| | AACCAAACACAAGTGCAACCAGACCTGCCTCGACAGAATAGCTGCTGGTAC | |
| | CTTTGATGCAGGAGAATTTTCTCTCCCCACCTTTGATTCACTGAATATTACTG | |
| | CTGCATCTTTAAATGACGACGGATTGGACAATCATACTATACTGCTTTACTAC | |
| | TCAACTGCTGCCTCCAGTTTGGCTGTAACACTGATGATAGCTATCTTTGTTG | |
| | TTTATATGGTCTCCAGAGACAATGTTTCTTGCTCCATTTGTCTATAAGGAAAG | |
| | TTAAGCCCTGTATTTTCCTTTATTGTAGTGCTTGTTTGCTTGTTGTCATTACA | |
| | AAGAAACGTTATTGAAAAATTGATGA | |
| Pan-CoV- | ATGTTCGTGTTCCTGGTGCTGCTGCCCCTGGTGAGCAGCCAGTGCGTGAAC | 49 |
| Flu | CTGACCACCAGGACCCAGCTGCCCCCCGCCTACACCAACAGCTTCACCAG | |
| Vaccine | GGGCGTGTACTACCCCGACAAGGTGTTCAGGAGCAGCGTGCTGCACAGCA | |
| #29 CoV- | CCCAGGACCTGTTCCTGCCCTTCTTCAGCAACGTGACCTGGTTCCACGCCA | |
| Wuhan | TCCACGTGAGCGGCACCAACGGCACCAAGAGGTTCGACAACCCCGTGCTG | |
| Spike | CCCTTCAACGACGGCGTGTACTTCGCCAGCACCGAGAAGAGCAACATCATC | |
| (HexaPro)- | AGGGGCTGGATCTTCGGCACCACCCTGGACAGCAAGACCCAGAGCCTGCT | |
| Influenza | GATCGTGAACAACGCCACCAACGTGGTGATCAAGGTGTGCGAGTTCCAGTT | |
| (HA) | CTGCAACGACCCCTTCCTGGGCGTGTACTACCACAAGAACAACAAGAGCTG | |
| | GATGGAGAGCGAGTTCAGGGTGTACAGCAGCGCCAACAACTGCACCTTCG | |
| | AGTACGTGAGCCAGCCCTTCCTGATGGACCTGGAGGGCAAGCAGGGCAAC | |
| | TTCAAGAACCTGAGGGAGTTCGTGTTCAAGAACATCGACGGCTACTTCAAG | |
| | ATCTACAGCAAGCACACCCCCATCAACCTGGTGAGGGACCTGCCCCAGGG | |
| | CTTCAGCGCCCTGGAGCCCCTGGTGGACCTGCCCATCGGCATCAACATCA | |
| | CCAGGTTCCAGACCCTGCTGGCCCTGCACAGGAGCTACCTGACCCCCGGC | |
| | GACAGCAGCAGCGGCTGGACCGCCGGCGCCGCCGCCTACTACGTGGGCT | |
| | ACCTGCAGCCCAGGACCTTCCTGCTGAAGTACAACGAGAACGGCACCATCA | |
| | CCGACGCCGTGGACTGCGCCCTGGACCCCCTGAGCGAGACCAAGTGCACC | |
| | CTGAAGAGCTTCACCGTGGAGAAGGGCATCTACCAGACCAGCAACTTCAGG | |
| | GTGCAGCCCACCGAGAGCATCGTGAGGTTCCCCAACATCACCAACCTGTGC | |
| | CCCTTCGGCGAGGTGTTCAACGCCACCAGGTTCGCCAGCGTGTACGCCTG | |
| | GAACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCAAG | |
| | CTGAACGACCTGTGCTTCACCAACGTGTACGCCGACAGCTTCGTGATCAGG | |
| | GGCGACGAGGTGAGGCAGATCGCCCCCGGCCAGACCGGCAAGATCGCCG | |
| | ACTACAACTACAAGCTGCCCGACGACTTCACCGGCTGCGTGATCGCCTGGA | |
| | ACAGCAACAACCTGGACAGCAAGGTGGGGGCAACTACAACTACCTGTACA | |
| | GGCTGTTCAGGAAGAGCAACCTGAAGCCCTTCGAGAGGGACATCAGCACC | |
| | GAGATCTACCAGGCCGGCAGCACCCCCTGCAACGGCGTGGAGGGCTTCAA | |
| | CTGCTACTTCCCCCTGCAGAGCTACGGCTTCCAGCCCACCAACGGCGTGG | |
| | GCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAA | |
| | GTGCGTGAACTTCAACTTCAACGGCCTGACCGGCACCGGCGTGCTGACCG | |
| | AGAGCAACAAGAAGTTCCTGCCCTTCCAGCAGTTCGGCAGGGACATCGCC | |
| | GACACCACCGACGCCGTGAGGGACCCCCAGACCCTGGAGATCCTGGACAT | |
| | CACCCCCTGCAGCTTCGGCGGCGTGAGCGTGATCACCCCCGGCACCAACA | |
| | CCAGCAACCAGGTGGCCGTGCTGTACCAGGACGTGAACTGCACCGAGGTG | |
| | CCCGTGGCCATCCACGCCGACCAGCTGACCCCCACCTGGAGGGTGTACAG | |
| | CACCGGCAGCAACGTGTTCCAGACCAGGGCCGGCTGCCTGATCGGCGCCG | |
| | AGCACGTGAACAACAGCTACGAGTGCGACATCCCCATCGGCGCCGGCATC | |
| | TGCGCCAGCTACCAGACCCAGACCAACAGCCCCGGCAGCGCCAGCAGCGT | |
| | GGCCAGCCAGAGCATCATCGCCTACACCATGAGCCTGGGCGCCGAGAACA | |
| | GCGTGGCCTACAGCAACAACAGCATCGCCATCCCCACCAACTTCACCATCA | |
| | GCGTGACCACCGAGATCCTGCCCGTGAGCATGACCAAGACCAGCGTGGAC | |
| | TGCACCATGTACATCTGCGGCGACAGCACCGAGTGCAGCAACCTGCTGCT | |
| | GCAGTACGGCAGCTTCTGCACCCAGCTGAACAGGGCCCTGACCGGCATCG | |
| | CCGTGGAGCAGGACAAGAACACCCAGGAGGTGTTCGCCCAGGTGAAGCAG | |
| | ATCTACAAGACCCCCCCCATCAAGGACTTCGGCGGCTTCAACTTCAGCCAG | |
| | ATCCTGCCCGACCCCAGCAAGCCCAGCAAGAGGAGCCCCATCGAGGACCT | |
| | GCTGTTCAACAAGGTGACCCTGGCCGACGCCGGCTTCATCAAGCAGTACG | |
| | GCGACTGCCTGGGCGACATCGCCGCCAGGGACCTGATCTGCGCCCAGAAG | |
| | TTCAACGGCCTGACCGTGCTGCCCCCCCTGCTGACCGACGAGATGATCGC | |
| | CCAGTACACCAGCGCCCTGCTGGCCGGCACCATCACCAGCGGCTGGACCT | |
| | TCGGCGCCGGCCCCGCCCTGCAGATCCCCTTCCCCATGCAGATGGCCTAC | |
| | AGGTTCAACGGCATCGGCGTGACCCAGAACGTGCTGTACGAGAACCAGAA | |
| | GCTGATCGCCAACCAGTTCAACAGCGCCATCGGCAAGATCCAGGACAGCCT | |
| | GAGCAGCACCCCCAGCGCCCTGGGCAAGCTGCAGGACGTGGTGAACCAGA | |
| | ACGCCCAGGCCCTGAACACCCTGGTGAAGCAGCTGAGCAGCAACTTCGGC | |
| | GCCATCAGCAGCGTGCTGAACGACATCCTGAGCAGGCTGGACCCCCCCGA | |
| | GGCCGAGGTGCAGATCGACAGGCTGATCACCGGCAGGCTGCAGAGCCTGC | |
| | AGACCTACGTGACCCAGCAGCTGATCAGGGCCGCCGAGATCAGGGCCAGC | |
| | GCCAACCTGGCCGCCACCAAGATGAGCGAGTGCGTGCTGGGCCAGAGCAA | |
| | GAGGGTGGACTTCTGCGGCAAGGGCTACCACCTGATGAGCTTCCCCCAGA | |
| | GCGCCCCCCACGGCGTGGTGTTCCTGCACGTGACCTACGTGCCCGCCCAG | |
| | GAGAAGAACTTCACCACCGCCCCCGCCATCTGCCACGACGGCAAGGCCCA | |
| | CTTCCCCAGGGAGGGCGTGTTCGTGAGCAACGGCACCCACTGGTTCGTGA | |
| | CCCAGAGGAACTTCTACGAGCCCCAGATCATCACCACCGACAACACCTTCG | |
| | TGAGCGGCAACTGCGACGTGGTGATCGGCATCGTGAACAACACCGTGTAC | |
| | GACCCCCTGCAGCCCGAGCTGGACAGCTTCAAGGAGGAGCTGGACAAGTA | |
| | CTTCAAGAACCACACCAGCCCCGACGTGGACCTGGGCGACATCAGCGGCA | |
| | TCAACGCCAGCGTGGTGAACATCCAGAAGGAGATCGACAGGCTGAACGAG | |
| | GTGGCCAAGAACCTGAACGAGAGCCTGATCGACCTGCAGGAGCTGGGCAA | |
| | GTACGAGCAGTACATCAAGTGGCCCTGGTACATCTGGCTGGGCTTCATCGC | |
| | CGGCCTGATCGCCATCGTGATGGTGACCATCATGCTGTGCTGCATGACCAG | |
| | CTGCTGCAGCTGCCTGAAGGGCTGCTGCAGCTGCGGCAGCTGCTGCAAGT | |
| | TCGACGAGGACGACAGCGAGCCCGTGCTGAAGGGCGTGAAGCTGCACTAC | |
| | ACCGGAAGCGGAGCCACGAACTTCTCTCTGTTAAAGCAAGCAGGAGATGTT | |
| | GAAGAAAACCCCGGGCCTATGAAGGCAATACTAGTAGTTCTGCTATATACAT | |
| | TTGCAACCGCAAATGCAGACACATTATGTATAGGTTATCATGCGAACAACTC | |
| | AACAGACACTGTAGACACAGTACTAGAAAAGAATGTAACAGTAACACACTCT | |
| | GTTAACCTTCTAGAAGACAAGCATAACGGGAAACTATGCAAACTAAGAGGG | |
| | GTAGCCCCATTGCATTTGGGTAAATGTAACATTGCTGGCTGGATCCTGGGA | |
| | AATCCAGAGTGTGAATCACTCTCCACAGCAAGCTCATGGTCCTACATTGTGG | |
| | AAACATCTAGTTCAGACAATGGAACGTGTTACCCAGGAGATTTCATCGATTA | |
| | TGAGGAGCTAAGAGAGCAATTGAGCTCAGTGTCATCATTTGAAAGGTTTGA | |
| | GATATTCCCCAAGACAAGTTCATGGCCCAATCATGACTCGAACAAAGGTGTA | |
| | ACGACAGCATGTCCTCATGCTGGAGCAAAAAGCTTCTACAAAAATTTAATAT | |
| | GGCTAGTTAAAAAAGGAAATTCATACCCAAAGCTCAGCAAATCCTACATTAA | |
| | TGATAAAGGGAAAGAAGTCCTCGTGCTATGGGGCATTCACCATCCACCTAC | |
| | TAGTGCTGACCAACAAAGTCTCTATCAGAATGCAGATGCATATGTTTTTGTG | |
| | GGGACATCAAGATACAGCAAGAAGTTCAAGCCGGAAATAGCAATAAGGCCC | |
| | AAAGTGAGGGATCAAGAAGGGAGAATGAACTATTACTGGACACTAGTAGAG | |
| | CCGGGAGACAAAATAACATTCGAAGCAACTGGAAATCTAGTGGTACCGAGA | |
| | TATGCATTCGCAATGGAAAGAAATGCTGGATCTGGTATTATCATTTCAGATA | |
| | CACCAGTCCACGATTGCAATACAACTTGTCAGACACCCAAGGGTGCTATAAA | |
| | CACCAGCCTCCCATTTCAGAACATACATCCGATCACAATTGGAAAATGTCCA | |
| | AAATATGTAAAAAGCACAAAATTGAGACTGGCCACAGGATTGAGGAATGTCC | |
| | CGTCCATTCAATCTAGAGGCCTATTTGGGGCCATTGCCGGTTTCATTGAAG | |
| | GGGGGTGGACAGGGATGGTAGATGGATGGTACGGTTATCACCATCAAAATG | |
| | AGCAGGGGTCAGGATATGCAGCCGACCTGAAGAGCACACAGAATGCCATT | |
| | GACGAGATTACTAACAAAGTAAACTCTGTTATTGAAAAAATGAATACACAGTT | |
| | CACAGCAGTAGGTAAAGAGTTCAACCACCTGGAAAAAAGAATAGAGAATTTA | |
| | AATAAAAAAGTTGATGATGGTTTCCTGGACATTTGGACTTACAATGCCGAAC | |
| | TGTTGGTTCTATTGGAAAATGAAAGAACTTTGGACTACCACGATTCAAAGGT | |
| | GAAGAACTTATATGAAAAGGTAAGAAGCCAGTTAAAAAACAATGCCAAGGAA | |
| | ATTGGAAACGGCTGCTTTGAATTTTACCACAAATGTGATAACACGTGCATGG | |
| | AAAGTGTCAAAAATGGGACTTATGACTACCCAAAATACTCAGAGGAAGCAAA | |
| | ATTAAACAGAGAAGAAATAGATGGGGTAAAGCTGGAATCAACAAGGATTTAC | |
| | CAGATTTTGGCGATCTATTCAACCGTCGCCAGTTCATTGGTACTGGTAGTCT | |
| | CCCTGGGGGCAATCAGTTTCTGGATGTGCTCTAATGGGTCTCTACAGTGTA | |
| | GAATATGTATTTAAGGAAGCGGAGCCACGAACTTCTCTCTGTTAAAGCAAGC | |
| | AGGAGATGTTGAAGAAAACCCCGGGCCTAGCAAAAGCAGGGGATAATTCTA | |
| | TTAACCATGAAGACTATCATTGCTTTGAGCTACATTCTATGTCTGGTTTTCGC | |
| | TCAAAAACTTCCTGGAAATGACAATAGCACTGCAACGCTGTGCCTTGGGCA | |
| | CCATGCAGTACCAAACGGAACGATAGTGAAAACAATCACGAATGACCAAATT | |
| | GAAGTTACTAATGCTACTGAGCTGGTTCAGAATTCCTCAATAGGTGAAATAT | |
| | GCGACAGTCCTCATCAGATCCTTGATGGAGAAAACTGCACACTAATAGATG | |
| | CTCTATTGGGAGACCCTCAGTGTGATGGCTTTCAAAATAAGAAATGGGACCT | |
| | TTTTGTTGAACGAAGCAAAGCCTACAGCAACTGTTACCCTTATGATGTGCCG | |
| | GATTATGCCTCCCTTAGGTCACTAGTTGCCTCATCCGGCACACTGGAGTTTA | |
| | ACAATGAAAGCTTCAATTGGGCTGGAGTCACTCAAAACGGAACAAGTTCTG | |
| | CTTGCATAAGGGGATCTAATAGTAGTTTCTTTAGTAGATTAAATTGGTTGACC | |
| | CACTTAAACTTCAAGTACCCAGCATTGAACGTGACTATGCCAAACAATGAAC | |
| | AATTTGACAAATTGTACATTTGGGGGGTTCACCACCCGGGTACGGACAAGG | |
| | ACCAAATCTTCCTGTATGCTCAATCATCAGGAAGAATCACAGTATCTACCAA | |
| | AAGAAGCCAACAAGCTGTAATCCCGAATATCGGATCTAGACCCAGAATAAG | |
| | GAATATCCCTAGCAGAATAAGCATCTATTGGACAATAGTAAAACCGGGAGAC | |
| | ATACTTTTGATTAACAGCACAGGGAATCTAATTGCTCCTAGGGGTTACTTCA | |
| | AAATACGAAGTGGGAAAAGCTCAATAATGAGATCAGATGCACCCATTGGCA | |
| | AATGCAAGTCTGAATGCATCACTCCAAATGGAAGCATTCCCAATGACAAACC | |
| | ATTCCAAAATGTAAACAGGATCACATACGGGGCCTGTCCCAGATATGTTAAG | |
| | CAAAGCACTCTGAAATTGGCAACAGGAATGCGAAATGTACCAGAGAAACAA | |
| | ACTAGAGGCATATTTGGCGCAATAGCGGGTTTCATAGAAAATGGTTGGGAG | |
| | GGAATGGTGGATGGTTGGTACGGTTTCAGGCATCAAAATTCTGAGGGAAGA | |
| | GGACAAGCAGCAGATCTCAAAAGCACTCAAGCAGCAATCGATCAAATCAAT | |
| | GGGAAGCTGAATCGATTGATCGGGAAAACCAACGAGAAATTCCATCAGATT | |
| | GAGAAAGAATTCTCAGAAGTAGAAGGGAGAATTCAGGACCTTGAGAAATAT | |
| | GTTGAGGACACAAAAATAGATCTCTGGTCATACAACGCAGAGCTTCTTGTTG | |
| | CCCTGGAAAACCAACATACAATTGATCTAACTGACTCAGAAATGAACAAACT | |
| | GTTTGAAAAAACAAAGAAGCAACTGAGGGAAAATGCTGAGGATATGGGCAA | |
| | TGGTTGTTTCAAAATATACCACAAATGTGACAATGCCTGCATAGGATCAATC | |
| | AGAAATGGAACTTATGACCACGATGTATACAGGGATGAAGCATTAAACAACC | |
| | GGTTCCAGATCAAGGGAGTTGAGCTGAAGTCAGGGTACAAAGATTGGATCC | |
| | TATGGATTTCCTTTGCCATATCATGTTTTTTGCTTTGTGTTGCTTTGTTGGGG | |
| | TTCATCATGTGGGCCTGCCAAAAGGGCAACATTAGGTGCAACATTTGCATTT | |
| | GAGTGCATTAATTAAAAACACCCTTGTTTCTACTGGAAGCGGAGCCACGAAC | |
| | TTCTCTCTGTTAAAGCAAGCAGGAGATGTTGAAGAAAACCCCGGGCCTATTT | |
| | TCTAATATCCACAAAATGAAGGCAATAATTGTACTACTCATGGTAGTAACATC | |
| | CAATGCAGATCGAATCTGCACTGGGATAACATCGTCAAACTCACCACATGTC | |
| | GTCAAAACTGCTACTCAAGGGGAGGTCAACGTGACCGGTGTAATACCACTG | |
| | ACAACAACACCCACCAAATCTCATTTTGCAAATCTCAAAGGAACAGAAACCA | |
| | GGGGGAAACTATGCCCAAAATGCCTCAACTGCACAGATCTGGATGTAGCCT | |
| | TGGGCAGACCAAAATGCACAGGGAAAATACCCTCTGCAAGGGTTTCAATAC | |
| | TCCATGAAGTCAGACCTGTTACATCTGGGTGCTTTCCTATAATGCACGATAG | |
| | AACAAAAATTAGACAGCTGCCTAACCTTCTCCGAGGATACGAACATGTCAGG | |
| | TTATCAACTCACAACGTTATCAATGCAAAAGATGCACCAGGAAGACCCTACG | |
| | AAATTGGAACCTCAGGGTCTTGCCCTAACATTACCAATGGAAACGGATTCTT | |
| | CGCAACAATGGCTTGGGCCGTCCCAAAAAACAAAACAGCAACAAATCCATT | |
| | AACAATAGAAGTACCATACATTTGTACAGAAGGAGAAGACCAAATTACCGTT | |
| | TGGGGGTTCCACTCTGACAACGAGACCCAAATGGCAAAGCTCTATGGGGAC | |
| | TCAAAGCCCCAGAAGTTCACCTCATCTGCCAACGGAGTGACCACACATTAC | |
| | GTTTCACAGATTGGTGGCTTCCCAAATCAAACAGAAGACGGAGGACTACCA | |
| | CAAAGTGGCAGAATTGTTGTTGATTACATGGTGCAGAAATCTGGAAAAACAG | |
| | GAACAATTACCTATCAAAGAGGTATTTTATTGCCTCAAAAAGTGTGGTGCGC | |
| | AAGTGGCAGGAGCAAGGTAATAAAAGGATCCTTGCCCTTAATTGGAGAAGC | |
| | AGATTGCCTCCATGAAAAATACGGTGGATTAAACAAAAGCAAGCCTTACTAC | |
| | ACAGGGGAACATGCAAAGGCCATAGGAAATTGCCCAATATGGGTGAAAACA | |
| | CCCTTGAAGCTGGCCAATGGAACCAAATATAGACCCCCTGCAAAACTATTAA | |
| | AGGAAAGAGGTTTCTTCGGAGCCATTGCTGGTTTCTTAGAGGGAGGATGGG | |
| | AAGGAATGATTGCAGGTTGGCACGGATACACATCCCATGGGGCACATGGAG | |
| | TAGCGGTGGCAGCTGACCTTAAGAGCACTCAAGAGGCCATAAACAAGATAA | |
| | CAAAAAATCTCAACTCTTTGAGTGAGCTGGAAGTAAAGAATCTTCAAAGACT | |
| | AAGCGGTGCCATGGATGAACTCCACAACGAAATACTAGAACTAGATGAGAA | |
| | AGTGGATGATCTCAGAGCTGACACAATAAGCTCACAAATAGAACTCGCAGT | |
| | CCTGCTTTCCAATGAAGGAATAATAAACAGTGAAGATGAACATCTCTTGGCG | |
| | CTTGAAAGAAAGCTGAAGAAAATGCTGGGCCCCTCTGCTGTAGAGATAGGG | |
| | AATGGATGCTTTGAAACCAAACACAAGTGCAACCAGACCTGCCTCGACAGA | |
| | ATAGCTGCTGGTACCTTTGATGCAGGAGAATTTTCTCTCCCCACCTTTGATT | |
| | CACTGAATATTACTGCTGCATCTTTAAATGACGACGGATTGGACAATCATAC | |
| | TATACTGCTTTACTACTCAACTGCTGCCTCCAGTTTGGCTGTAACACTGATG | |
| | ATAGCTATCTTTGTTGTTTATATGGTCTCCAGAGACAATGTTTCTTGCTCCAT | |
| | TTGTCTATAAGGAAAGTTAAGCCCTGTATTTTCCTTTATTGTAGTGCTTGTTT | |
| | GCTTGTTGTCATTACAAAGAAACGTTATTGAAAAATTGATGA | |
| Pan-CoV- | ATGTTCGTGTTCCTGGTGCTGCTGCCCCTGGTGAGCAGCCAGTGCGTGAAC | 50 |
| Flu | CTGACCACCAGGACCCAGCTGCCCCCCGCCTACACCAACAGCTTCACCAG | |
| Vaccine | GGGCGTGTACTACCCCGACAAGGTGTTCAGGAGCAGCGTGCTGCACAGCA | |
| #30 CoV- | CCCAGGACCTGTTCCTGCCCTTCTTCAGCAACGTGACCTGGTTCCACGCCA | |
| Wuhan | TCCACGTGAGCGGCACCAACGGCACCAAGAGGTTCGACAACCCCGTGCTG | |
| Spike | CCCTTCAACGACGGCGTGTACTTCGCCAGCACCGAGAAGAGCAACATCATC | |
| (2 proline | AGGGGCTGGATCTTCGGCACCACCCTGGACAGCAAGACCCAGAGCCTGCT | |
| subst)- | GATCGTGAACAACGCCACCAACGTGGTGATCAAGGTGTGCGAGTTCCAGTT | |
| Influenza | CTGCAACGACCCCTTCCTGGGCGTGTACTACCACAAGAACAACAAGAGCTG | |
| (HA) | GATGGAGAGCGAGTTCAGGGTGTACAGCAGCGCCAACAACTGCACCTTCG | |
| | AGTACGTGAGCCAGCCCTTCCTGATGGACCTGGAGGGCAAGCAGGGCAAC | |
| | TTCAAGAACCTGAGGGAGTTCGTGTTCAAGAACATCGACGGCTACTTCAAG | |
| | ATCTACAGCAAGCACACCCCCATCAACCTGGTGAGGGACCTGCCCCAGGG | |
| | CTTCAGCGCCCTGGAGCCCCTGGTGGACCTGCCCATCGGCATCAACATCA | |
| | CCAGGTTCCAGACCCTGCTGGCCCTGCACAGGAGCTACCTGACCCCCGGC | |
| | GACAGCAGCAGCGGCTGGACCGCCGGCGCCGCCGCCTACTACGTGGGCT | |
| | ACCTGCAGCCCAGGACCTTCCTGCTGAAGTACAACGAGAACGGCACCATCA | |
| | CCGACGCCGTGGACTGCGCCCTGGACCCCCTGAGCGAGACCAAGTGCACC | |
| | CTGAAGAGCTTCACCGTGGAGAAGGGCATCTACCAGACCAGCAACTTCAGG | |
| | GTGCAGCCCACCGAGAGCATCGTGAGGTTCCCCAACATCACCAACCTGTGC | |
| | CCCTTCGGCGAGGTGTTCAACGCCACCAGGTTCGCCAGCGTGTACGCCTG | |
| | GAACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCAAG | |
| | CTGAACGACCTGTGCTTCACCAACGTGTACGCCGACAGCTTCGTGATCAGG | |
| | GGCGACGAGGTGAGGCAGATCGCCCCCGGCCAGACCGGCAAGATCGCCG | |
| | ACTACAACTACAAGCTGCCCGACGACTTCACCGGCTGCGTGATCGCCTGGA | |
| | ACAGCAACAACCTGGACAGCAAGGTGGGCGGCAACTACAACTACCTGTACA | |
| | GGCTGTTCAGGAAGAGCAACCTGAAGCCCTTCGAGAGGGACATCAGCACC | |
| | GAGATCTACCAGGCCGGCAGCACCCCCTGCAACGGCGTGGAGGGCTTCAA | |
| | CTGCTACTTCCCCCTGCAGAGCTACGGCTTCCAGCCCACCAACGGCGTGG | |
| | GCTACCAGCCCTACAGGGTGGTGGTGCTGAGCTTCGAGCTGCTGCACGCC | |
| | CCCGCCACCGTGTGCGGCCCCAAGAAGAGCACCAACCTGGTGAAGAACAA | |
| | GTGCGTGAACTTCAACTTCAACGGCCTGACCGGCACCGGCGTGCTGACCG | |
| | AGAGCAACAAGAAGTTCCTGCCCTTCCAGCAGTTCGGCAGGGACATCGCC | |
| | GACACCACCGACGCCGTGAGGGACCCCCAGACCCTGGAGATCCTGGACAT | |
| | CACCCCCTGCAGCTTCGGCGGCGTGAGCGTGATCACCCCCGGCACCAACA | |
| | CCAGCAACCAGGTGGCCGTGCTGTACCAGGACGTGAACTGCACCGAGGTG | |
| | CCCGTGGCCATCCACGCCGACCAGCTGACCCCCACCTGGAGGGTGTACAG | |
| | CACCGGCAGCAACGTGTTCCAGACCAGGGCCGGCTGCCTGATCGGCGCCG | |
| | AGCACGTGAACAACAGCTACGAGTGCGACATCCCCATCGGCGCCGGCATC | |
| | TGCGCCAGCTACCAGACCCAGACCAACAGCCCCAGGAGGGCCAGGAGCGT | |
| | GGCCAGCCAGAGCATCATCGCCTACACCATGAGCCTGGGCGCCGAGAACA | |
| | GCGTGGCCTACAGCAACAACAGCATCGCCATCCCCACCAACTTCACCATCA | |
| | GCGTGACCACCGAGATCCTGCCCGTGAGCATGACCAAGACCAGCGTGGAC | |
| | TGCACCATGTACATCTGCGGCGACAGCACCGAGTGCAGCAACCTGCTGCT | |
| | GCAGTACGGCAGCTTCTGCACCCAGCTGAACAGGGCCCTGACCGGCATCG | |
| | CCGTGGAGCAGGACAAGAACACCCAGGAGGTGTTCGCCCAGGTGAAGCAG | |
| | ATCTACAAGACCCCCCCCATCAAGGACTTCGGCGGCTTCAACTTCAGCCAG | |
| | ATCCTGCCCGACCCCAGCAAGCCCAGCAAGAGGAGCTTCATCGAGGACCT | |
| | GCTGTTCAACAAGGTGACCCTGGCCGACGCCGGCTTCATCAAGCAGTACG | |
| | GCGACTGCCTGGGCGACATCGCCGCCAGGGACCTGATCTGCGCCCAGAAG | |
| | TTCAACGGCCTGACCGTGCTGCCCCCCCTGCTGACCGACGAGATGATCGC | |
| | CCAGTACACCAGCGCCCTGCTGGCCGGCACCATCACCAGCGGCTGGACCT | |
| | TCGGCGCCGGCGCCGCCCTGCAGATCCCCTTCGCCATGCAGATGGCCTAC | |
| | AGGTTCAACGGCATCGGCGTGACCCAGAACGTGCTGTACGAGAACCAGAA | |
| | GCTGATCGCCAACCAGTTCAACAGCGCCATCGGCAAGATCCAGGACAGCCT | |
| | GAGCAGCACCGCCAGCGCCCTGGGCAAGCTGCAGGACGTGGTGAACCAG | |
| | AACGCCCAGGCCCTGAACACCCTGGTGAAGCAGCTGAGCAGCAACTTCGG | |
| | CGCCATCAGCAGCGTGCTGAACGACATCCTGAGCAGGCTGGACCCCCCCG | |
| | AGGCCGAGGTGCAGATCGACAGGCTGATCACCGGCAGGCTGCAGAGCCTG | |
| | CAGACCTACGTGACCCAGCAGCTGATCAGGGCCGCCGAGATCAGGGCCAG | |
| | CGCCAACCTGGCCGCCACCAAGATGAGCGAGTGCGTGCTGGGCCAGAGCA | |
| | AGAGGGTGGACTTCTGCGGCAAGGGCTACCACCTGATGAGCTTCCCCCAG | |
| | AGCGCCCCCCACGGCGTGGTGTTCCTGCACGTGACCTACGTGCCCGCCCA | |
| | GGAGAAGAACTTCACCACCGCCCCCGCCATCTGCCACGACGGCAAGGCCC | |
| | ACTTCCCCAGGGAGGGCGTGTTCGTGAGCAACGGCACCCACTGGTTCGTG | |
| | ACCCAGAGGAACTTCTACGAGCCCCAGATCATCACCACCGACAACACCTTC | |
| | GTGAGCGGCAACTGCGACGTGGTGATCGGCATCGTGAACAACACCGTGTA | |
| | CGACCCCCTGCAGCCCGAGCTGGACAGCTTCAAGGAGGAGCTGGACAAGT | |
| | ACTTCAAGAACCACACCAGCCCCGACGTGGACCTGGGCGACATCAGCGGC | |
| | ATCAACGCCAGCGTGGTGAACATCCAGAAGGAGATCGACAGGCTGAACGA | |
| | GGTGGCCAAGAACCTGAACGAGAGCCTGATCGACCTGCAGGAGCTGGGCA | |
| | AGTACGAGCAGTACATCAAGTGGCCCTGGTACATCTGGCTGGGCTTCATCG | |
| | CCGGCCTGATCGCCATCGTGATGGTGACCATCATGCTGTGCTGCATGACCA | |
| | GCTGCTGCAGCTGCCTGAAGGGCTGCTGCAGCTGCGGCAGCTGCTGCAAG | |
| | TTCGACGAGGACGACAGCGAGCCCGTGCTGAAGGGCGTGAAGCTGCACTA | |
| | CACCGGAAGCGGAGCCACGAACTTCTCTCTGTTAAAGCAAGCAGGAGATGT | |
| | TGAAGAAAACCCCGGGCCTATGAAGGCAATACTAGTAGTTCTGCTATATACA | |
| | TTTGCAACCGCAAATGCAGACACATTATGTATAGGTTATCATGCGAACAACT | |
| | CAACAGACACTGTAGACACAGTACTAGAAAAGAATGTAACAGTAACACACTC | |
| | TGTTAACCTTCTAGAAGACAAGCATAACGGGAAACTATGCAAACTAAGAGGG | |
| | GTAGCCCCATTGCATTTGGGTAAATGTAACATTGCTGGCTGGATCCTGGGA | |
| | AATCCAGAGTGTGAATCACTCTCCACAGCAAGCTCATGGTCCTACATTGTGG | |
| | AAACATCTAGTTCAGACAATGGAACGTGTTACCCAGGAGATTTCATCGATTA | |
| | TGAGGAGCTAAGAGAGCAATTGAGCTCAGTGTCATCATTTGAAAGGTTTGA | |
| | GATATTCCCCAAGACAAGTTCATGGCCCAATCATGACTCGAACAAAGGTGTA | |
| | ACGACAGCATGTCCTCATGCTGGAGCAAAAAGCTTCTACAAAAATTTAATAT | |
| | GGCTAGTTAAAAAAGGAAATTCATACCCAAAGCTCAGCAAATCCTACATTAA | |
| | TGATAAAGGGAAAGAAGTCCTCGTGCTATGGGGCATTCACCATCCACCTAC | |
| | TAGTGCTGACCAACAAAGTCTCTATCAGAATGCAGATGCATATGTTTTTGTG | |
| | GGGACATCAAGATACAGCAAGAAGTTCAAGCCGGAAATAGCAATAAGGCCC | |
| | AAAGTGAGGGATCAAGAAGGGAGAATGAACTATTACTGGACACTAGTAGAG | |
| | CCGGGAGACAAAATAACATTCGAAGCAACTGGAAATCTAGTGGTACCGAGA | |
| | TATGCATTCGCAATGGAAAGAAATGCTGGATCTGGTATTATCATTTCAGATA | |
| | CACCAGTCCACGATTGCAATACAACTTGTCAGACACCCAAGGGTGCTATAAA | |
| | CACCAGCCTCCCATTTCAGAACATACATCCGATCACAATTGGAAAATGTCCA | |
| | AAATATGTAAAAAGCACAAAATTGAGACTGGCCACAGGATTGAGGAATGTCC | |
| | CGTCCATTCAATCTAGAGGCCTATTTGGGGCCATTGCCGGTTTCATTGAAG | |
| | GGGGGTGGACAGGGATGGTAGATGGATGGTACGGTTATCACCATCAAAATG | |
| | AGCAGGGGTCAGGATATGCAGCCGACCTGAAGAGCACACAGAATGCCATT | |
| | GACGAGATTACTAACAAAGTAAACTCTGTTATTGAAAAAATGAATACACAGTT | |
| | CACAGCAGTAGGTAAAGAGTTCAACCACCTGGAAAAAAGAATAGAGAATTTA | |
| | AATAAAAAAGTTGATGATGGTTTCCTGGACATTTGGACTTACAATGCCGAAC | |
| | TGTTGGTTCTATTGGAAAATGAAAGAACTTTGGACTACCACGATTCAAAGGT | |
| | GAAGAACTTATATGAAAAGGTAAGAAGCCAGTTAAAAAACAATGCCAAGGAA | |
| | ATTGGAAACGGCTGCTTTGAATTTTACCACAAATGTGATAACACGTGCATGG | |
| | AAAGTGTCAAAAATGGGACTTATGACTACCCAAAATACTCAGAGGAAGCAAA | |
| | ATTAAACAGAGAAGAAATAGATGGGGTAAAGCTGGAATCAACAAGGATTTAC | |
| | CAGATTTTGGCGATCTATTCAACCGTCGCCAGTTCATTGGTACTGGTAGTCT | |
| | CCCTGGGGGCAATCAGTTTCTGGATGTGCTCTAATGGGTCTCTACAGTGTA | |
| | GAATATGTATTTAAGGAAGCGGAGCCACGAACTTCTCTCTGTTAAAGCAAGC | |
| | AGGAGATGTTGAAGAAAACCCCGGGCCTAGCAAAAGCAGGGGATAATTCTA | |
| | TTAACCATGAAGACTATCATTGCTTTGAGCTACATTCTATGTCTGGTTTTCGC | |
| | TCAAAAACTTCCTGGAAATGACAATAGCACTGCAACGCTGTGCCTTGGGCA | |
| | CCATGCAGTACCAAACGGAACGATAGTGAAAACAATCACGAATGACCAAATT | |
| | GAAGTTACTAATGCTACTGAGCTGGTTCAGAATTCCTCAATAGGTGAAATAT | |
| | GCGACAGTCCTCATCAGATCCTTGATGGAGAAAACTGCACACTAATAGATG | |
| | CTCTATTGGGAGACCCTCAGTGTGATGGCTTTCAAAATAAGAAATGGGACCT | |
| | TTTTGTTGAACGAAGCAAAGCCTACAGCAACTGTTACCCTTATGATGTGCCG | |
| | GATTATGCCTCCCTTAGGTCACTAGTTGCCTCATCCGGCACACTGGAGTTTA | |
| | ACAATGAAAGCTTCAATTGGGCTGGAGTCACTCAAAACGGAACAAGTTCTG | |
| | CTTGCATAAGGGGATCTAATAGTAGTTTCTTTAGTAGATTAAATTGGTTGACC | |
| | CACTTAAACTTCAAGTACCCAGCATTGAACGTGACTATGCCAAACAATGAAC | |
| | AATTTGACAAATTGTACATTTGGGGGGTTCACCACCCGGGTACGGACAAGG | |
| | ACCAAATCTTCCTGTATGCTCAATCATCAGGAAGAATCACAGTATCTACCAA | |
| | AAGAAGCCAACAAGCTGTAATCCCGAATATCGGATCTAGACCCAGAATAAG | |
| | GAATATCCCTAGCAGAATAAGCATCTATTGGACAATAGTAAAACCGGGAGAC | |
| | ATACTTTTGATTAACAGCACAGGGAATCTAATTGCTCCTAGGGGTTACTTCA | |
| | AAATACGAAGTGGGAAAAGCTCAATAATGAGATCAGATGCACCCATTGGCA | |
| | AATGCAAGTCTGAATGCATCACTCCAAATGGAAGCATTCCCAATGACAAACC | |
| | ATTCCAAAATGTAAACAGGATCACATACGGGGCCTGTCCCAGATATGTTAAG | |
| | CAAAGCACTCTGAAATTGGCAACAGGAATGCGAAATGTACCAGAGAAACAA | |
| | ACTAGAGGCATATTTGGCGCAATAGCGGGTTTCATAGAAAATGGTTGGGAG | |
| | GGAATGGTGGATGGTTGGTACGGTTTCAGGCATCAAAATTCTGAGGGAAGA | |
| | GGACAAGCAGCAGATCTCAAAAGCACTCAAGCAGCAATCGATCAAATCAAT | |
| | GGGAAGCTGAATCGATTGATCGGGAAAACCAACGAGAAATTCCATCAGATT | |
| | GAGAAAGAATTCTCAGAAGTAGAAGGGAGAATTCAGGACCTTGAGAAATAT | |
| | GTTGAGGACACAAAAATAGATCTCTGGTCATACAACGCAGAGCTTCTTGTTG | |
| | CCCTGGAAAACCAACATACAATTGATCTAACTGACTCAGAAATGAACAAACT | |
| | GTTTGAAAAAACAAAGAAGCAACTGAGGGAAAATGCTGAGGATATGGGCAA | |
| | TGGTTGTTTCAAAATATACCACAAATGTGACAATGCCTGCATAGGATCAATC | |
| | AGAAATGGAACTTATGACCACGATGTATACAGGGATGAAGCATTAAACAACC | |
| | GGTTCCAGATCAAGGGAGTTGAGCTGAAGTCAGGGTACAAAGATTGGATCC | |
| | TATGGATTTCCTTTGCCATATCATGTTTTTTGCTTTGTGTTGCTTTGTTGGGG | |
| | TTCATCATGTGGGCCTGCCAAAAGGGCAACATTAGGTGCAACATTTGCATTT | |
| | GAGTGCATTAATTAAAAACACCCTTGTTTCTACTGGAAGCGGAGCCACGAAC | |
| | TTCTCTCTGTTAAAGCAAGCAGGAGATGTTGAAGAAAACCCCGGGCCTATTT | |
| | TCTAATATCCACAAAATGAAGGCAATAATTGTACTACTCATGGTAGTAACATC | |
| | CAATGCAGATCGAATCTGCACTGGGATAACATCGTCAAACTCACCACATGTC | |
| | GTCAAAACTGCTACTCAAGGGGAGGTCAACGTGACCGGTGTAATACCACTG | |
| | ACAACAACACCCACCAAATCTCATTTTGCAAATCTCAAAGGAACAGAAACCA | |
| | GGGGGAAACTATGCCCAAAATGCCTCAACTGCACAGATCTGGATGTAGCCT | |
| | TGGGCAGACCAAAATGCACAGGGAAAATACCCTCTGCAAGGGTTTCAATAC | |
| | TCCATGAAGTCAGACCTGTTACATCTGGGTGCTTTCCTATAATGCACGATAG | |
| | AACAAAAATTAGACAGCTGCCTAACCTTCTCCGAGGATACGAACATGTCAGG | |
| | TTATCAACTCACAACGTTATCAATGCAAAAGATGCACCAGGAAGACCCTACG | |
| | AAATTGGAACCTCAGGGTCTTGCCCTAACATTACCAATGGAAACGGATTCTT | |
| | CGCAACAATGGCTTGGGCCGTCCCAAAAAACAAAACAGCAACAAATCCATT | |
| | AACAATAGAAGTACCATACATTTGTACAGAAGGAGAAGACCAAATTACCGTT | |
| | TGGGGGTTCCACTCTGACAACGAGACCCAAATGGCAAAGCTCTATGGGGAC | |
| | TCAAAGCCCCAGAAGTTCACCTCATCTGCCAACGGAGTGACCACACATTAC | |
| | GTTTCACAGATTGGTGGCTTCCCAAATCAAACAGAAGACGGAGGACTACCA | |
| | CAAAGTGGCAGAATTGTTGTTGATTACATGGTGCAGAAATCTGGAAAAACAG | |
| | GAACAATTACCTATCAAAGAGGTATTTTATTGCCTCAAAAAGTGTGGTGCGC | |
| | AAGTGGCAGGAGCAAGGTAATAAAAGGATCCTTGCCCTTAATTGGAGAAGC | |
| | AGATTGCCTCCATGAAAAATACGGTGGATTAAACAAAAGCAAGCCTTACTAC | |
| | ACAGGGGAACATGCAAAGGCCATAGGAAATTGCCCAATATGGGTGAAAACA | |
| | CCCTTGAAGCTGGCCAATGGAACCAAATATAGACCCCCTGCAAAACTATTAA | |
| | AGGAAAGAGGTTTCTTCGGAGCCATTGCTGGTTTCTTAGAGGGAGGATGGG | |
| | AAGGAATGATTGCAGGTTGGCACGGATACACATCCCATGGGGCACATGGAG | |
| | TAGCGGTGGCAGCTGACCTTAAGAGCACTCAAGAGGCCATAAACAAGATAA | |
| | CAAAAAATCTCAACTCTTTGAGTGAGCTGGAAGTAAAGAATCTTCAAAGACT | |
| | AAGCGGTGCCATGGATGAACTCCACAACGAAATACTAGAACTAGATGAGAA | |
| | AGTGGATGATCTCAGAGCTGACACAATAAGCTCACAAATAGAACTCGCAGT | |
| | CCTGCTTTCCAATGAAGGAATAATAAACAGTGAAGATGAACATCTCTTGGCG | |
| | CTTGAAAGAAAGCTGAAGAAAATGCTGGGCCCCTCTGCTGTAGAGATAGGG | |
| | AATGGATGCTTTGAAACCAAACACAAGTGCAACCAGACCTGCCTCGACAGA | |
| | ATAGCTGCTGGTACCTTTGATGCAGGAGAATTTTCTCTCCCCACCTTTGATT | |
| | CACTGAATATTACTGCTGCATCTTTAAATGACGACGGATTGGACAATCATAC | |
| | TATACTGCTTTACTACTCAACTGCTGCCTCCAGTTTGGCTGTAACACTGATG | |
| | ATAGCTATCTTTGTTGTTTATATGGTCTCCAGAGACAATGTTTCTTGCTCCAT | |
| | TTGTCTATAAGGAAAGTTAAGCCCTGTATTTTCCTTTATTGTAGTGCTTGTTT | |
| | GCTTGTTGTCATTACAAAGAAACGTTATTGAAAAATTGATGA | |
| Pan-CoV- | ATGATGAAGGCAATACTAGTAGTTCTGCTATATACATTTGCAACCGCAAATG | 51 |
| Flu | CAGACACATTATGTATAGGTTATCATGCGAACAACTCAACAGACACTGTAGA | |
| Vaccine | CACAGTACTAGAAAAGAATGTAACAGTAACACACTCTGTTAACCTTCTAGAA | |
| #31 (H1N1) | GACAAGCATAACGGGAAACTATGCAAACTAAGAGGGGTAGCCCCATTGCAT | |
| | TTGGGTAAATGTAACATTGCTGGCTGGATCCTGGGAAATCCAGAGTGTGAA | |
| | TCACTCTCCACAGCAAGCTCATGGTCCTACATTGTGGAAACATCTAGTTCAG | |
| | ACAATGGAACGTGTTACCCAGGAGATTTCATCGATTATGAGGAGCTAAGAG | |
| | AGCAATTGAGCTCAGTGTCATCATTTGAAAGGTTTGAGATATTCCCCAAGAC | |
| | AAGTTCATGGCCCAATCATGACTCGAACAAAGGTGTAACGACAGCATGTCC | |
| | TCATGCTGGAGCAAAAAGCTTCTACAAAAATTTAATATGGCTAGTTAAAAAAG | |
| | GAAATTCATACCCAAAGCTCAGCAAATCCTACATTAATGATAAAGGGAAAGA | |
| | AGTCCTCGTGCTATGGGGCATTCACCATCCACCTACTAGTGCTGACCAACA | |
| | AAGTCTCTATCAGAATGCAGATGCATATGTTTTTGTGGGGACATCAAGATAC | |
| | AGCAAGAAGTTCAAGCCGGAAATAGCAATAAGGCCCAAAGTGAGGGATCAA | |
| | GAAGGGAGAATGAACTATTACTGGACACTAGTAGAGCCGGGAGACAAAATA | |
| | ACATTCGAAGCAACTGGAAATCTAGTGGTACCGAGATATGCATTCGCAATG | |
| | GAAAGAAATGCTGGATCTGGTATTATCATTTCAGATACACCAGTCCACGATT | |
| | GCAATACAACTTGTCAGACACCCAAGGGTGCTATAAACACCAGCCTCCCATT | |
| | TCAGAACATACATCCGATCACAATTGGAAAATGTCCAAAATATGTAAAAAGC | |
| | ACAAAATTGAGACTGGCCACAGGATTGAGGAATGTCCCGTCCATTCAATCTA | |
| | GAGGCCTATTTGGGGCCATTGCCGGTTTCATTGAAGGGGGGTGGACAGGG | |
| | ATGGTAGATGGATGGTACGGTTATCACCATCAAAATGAGCAGGGGTCAGGA | |
| | TATGCAGCCGACCTGAAGAGCACACAGAATGCCATTGACGAGATTACTAAC | |
| | AAAGTAAACTCTGTTATTGAAAAAATGAATACACAGTTCACAGCAGTAGGTA | |
| | AAGAGTTCAACCACCTGGAAAAAAGAATAGAGAATTTAAATAAAAAAGTTGA | |
| | TGATGGTTTCCTGGACATTTGGACTTACAATGCCGAACTGTTGGTTCTATTG | |
| | GAAAATGAAAGAACTTTGGACTACCACGATTCAAAGGTGAAGAACTTATATG | |
| | AAAAGGTAAGAAGCCAGTTAAAAAACAATGCCAAGGAAATTGGAAACGGCT | |
| | GCTTTGAATTTTACCACAAATGTGATAACACGTGCATGGAAAGTGTCAAAAA | |
| | TGGGACTTATGACTACCCAAAATACTCAGAGGAAGCAAAATTAAACAGAGAA | |
| | GAAATAGATGGGGTAAAGCTGGAATCAACAAGGATTTACCAGATTTTGGCG | |
| | ATCTATTCAACCGTCGCCAGTTCATTGGTACTGGTAGTCTCCCTGGGGGCA | |
| | ATCAGTTTCTGGATGTGCTCTAATGGGTCTCTACAGTGTAGAATATGTATTTA | |
| | ATGATGA | |
| Pan-CoV- | ATGAGCAAAAGCAGGGGATAATTCTATTAACCATGAAGACTATCATTGCTTT | 52 |
| Flu | GAGCTACATTCTATGTCTGGTTTTCGCTCAAAAACTTCCTGGAAATGACAAT | |
| Vaccine | AGCACTGCAACGCTGTGCCTTGGGCACCATGCAGTACCAAACGGAACGATA | |
| #32 (H3N2) | GTGAAAACAATCACGAATGACCAAATTGAAGTTACTAATGCTACTGAGCTGG | |
| | TTCAGAATTCCTCAATAGGTGAAATATGCGACAGTCCTCATCAGATCCTTGA | |
| | TGGAGAAAACTGCACACTAATAGATGCTCTATTGGGAGACCCTCAGTGTGAT | |
| | GGCTTTCAAAATAAGAAATGGGACCTTTTTGTTGAACGAAGCAAAGCCTACA | |
| | GCAACTGTTACCCTTATGATGTGCCGGATTATGCCTCCCTTAGGTCACTAGT | |
| | TGCCTCATCCGGCACACTGGAGTTTAACAATGAAAGCTTCAATTGGGCTGG | |
| | AGTCACTCAAAACGGAACAAGTTCTGCTTGCATAAGGGGATCTAATAGTAGT | |
| | TTCTTTAGTAGATTAAATTGGTTGACCCACTTAAACTTCAAGTACCCAGCATT | |
| | GAACGTGACTATGCCAAACAATGAACAATTTGACAAATTGTACATTTGGGGG | |
| | GTTCACCACCCGGGTACGGACAAGGACCAAATCTTCCTGTATGCTCAATCA | |
| | TCAGGAAGAATCACAGTATCTACCAAAAGAAGCCAACAAGCTGTAATCCCGA | |
| | ATATCGGATCTAGACCCAGAATAAGGAATATCCCTAGCAGAATAAGCATCTA | |
| | TTGGACAATAGTAAAACCGGGAGACATACTTTTGATTAACAGCACAGGGAAT | |
| | CTAATTGCTCCTAGGGGTTACTTCAAAATACGAAGTGGGAAAAGCTCAATAA | |
| | TGAGATCAGATGCACCCATTGGCAAATGCAAGTCTGAATGCATCACTCCAAA | |
| | TGGAAGCATTCCCAATGACAAACCATTCCAAAATGTAAACAGGATCACATAC | |
| | GGGGCCTGTCCCAGATATGTTAAGCAAAGCACTCTGAAATTGGCAACAGGA | |
| | ATGCGAAATGTACCAGAGAAACAAACTAGAGGCATATTTGGCGCAATAGCG | |
| | GGTTTCATAGAAAATGGTTGGGAGGGAATGGTGGATGGTTGGTACGGTTTC | |
| | AGGCATCAAAATTCTGAGGGAAGAGGACAAGCAGCAGATCTCAAAAGCACT | |
| | CAAGCAGCAATCGATCAAATCAATGGGAAGCTGAATCGATTGATCGGGAAA | |
| | ACCAACGAGAAATTCCATCAGATTGAGAAAGAATTCTCAGAAGTAGAAGGGA | |
| | GAATTCAGGACCTTGAGAAATATGTTGAGGACACAAAAATAGATCTCTGGTC | |
| | ATACAACGCAGAGCTTCTTGTTGCCCTGGAAAACCAACATACAATTGATCTA | |
| | ACTGACTCAGAAATGAACAAACTGTTTGAAAAAACAAAGAAGCAACTGAGGG | |
| | AAAATGCTGAGGATATGGGCAATGGTTGTTTCAAAATATACCACAAATGTGA | |
| | CAATGCCTGCATAGGATCAATCAGAAATGGAACTTATGACCACGATGTATAC | |
| | AGGGATGAAGCATTAAACAACCGGTTCCAGATCAAGGGAGTTGAGCTGAAG | |
| | TCAGGGTACAAAGATTGGATCCTATGGATTTCCTTTGCCATATCATGTTTTTT | |
| | GCTTTGTGTTGCTTTGTTGGGGTTCATCATGTGGGCCTGCCAAAAGGGCAA | |
| | CATTAGGTGCAACATTTGCATTTGAGTGCATTAATTAAAAACACCCTTGTTTC | |
| | TACTTGATGA | |
| Pan-CoV- | ATGATTTTCTAATATCCACAAAATGAAGGCAATAATTGTACTACTCATGGTAG | 53 |
| Flu | TAACATCCAATGCAGATCGAATCTGCACTGGGATAACATCGTCAAACTCACC | |
| Vaccine | ACATGTCGTCAAAACTGCTACTCAAGGGGAGGTCAACGTGACCGGTGTAAT | |
| #33 (H3N2) | ACCACTGACAACAACACCCACCAAATCTCATTTTGCAAATCTCAAAGGAACA | |
| | GAAACCAGGGGGAAACTATGCCCAAAATGCCTCAACTGCACAGATCTGGAT | |
| | GTAGCCTTGGGCAGACCAAAATGCACAGGGAAAATACCCTCTGCAAGGGTT | |
| | TCAATACTCCATGAAGTCAGACCTGTTACATCTGGGTGCTTTCCTATAATGC | |
| | ACGATAGAACAAAAATTAGACAGCTGCCTAACCTTCTCCGAGGATACGAACA | |
| | TGTCAGGTTATCAACTCACAACGTTATCAATGCAAAAGATGCACCAGGAAGA | |
| | CCCTACGAAATTGGAACCTCAGGGTCTTGCCCTAACATTACCAATGGAAAC | |
| | GGATTCTTCGCAACAATGGCTTGGGCCGTCCCAAAAAACAAAACAGCAACA | |
| | AATCCATTAACAATAGAAGTACCATACATTTGTACAGAAGGAGAAGACCAAA | |
| | TTACCGTTTGGGGGTTCCACTCTGACAACGAGACCCAAATGGCAAAGCTCT | |
| | ATGGGGACTCAAAGCCCCAGAAGTTCACCTCATCTGCCAACGGAGTGACCA | |
| | CACATTACGTTTCACAGATTGGTGGCTTCCCAAATCAAACAGAAGACGGAG | |
| | GACTACCACAAAGTGGCAGAATTGTTGTTGATTACATGGTGCAGAAATCTGG | |
| | AAAAACAGGAACAATTACCTATCAAAGAGGTATTTTATTGCCTCAAAAAGTGT | |
| | GGTGCGCAAGTGGCAGGAGCAAGGTAATAAAAGGATCCTTGCCCTTAATTG | |
| | GAGAAGCAGATTGCCTCCATGAAAAATACGGTGGATTAAACAAAAGCAAGC | |
| | CTTACTACACAGGGGAACATGCAAAGGCCATAGGAAATTGCCCAATATGGG | |
| | TGAAAACACCCTTGAAGCTGGCCAATGGAACCAAATATAGACCCCCTGCAA | |
| | AACTATTAAAGGAAAGAGGTTTCTTCGGAGCCATTGCTGGTTTCTTAGAGGG | |
| | AGGATGGGAAGGAATGATTGCAGGTTGGCACGGATACACATCCCATGGGG | |
| | CACATGGAGTAGCGGTGGCAGCTGACCTTAAGAGCACTCAAGAGGCCATAA | |
| | ACAAGATAACAAAAAATCTCAACTCTTTGAGTGAGCTGGAAGTAAAGAATCT | |
| | TCAAAGACTAAGCGGTGCCATGGATGAACTCCACAACGAAATACTAGAACTA | |
| | GATGAGAAAGTGGATGATCTCAGAGCTGACACAATAAGCTCACAAATAGAA | |
| | CTCGCAGTCCTGCTTTCCAATGAAGGAATAATAAACAGTGAAGATGAACATC | |
| | TCTTGGCGCTTGAAAGAAAGCTGAAGAAAATGCTGGGCCCCTCTGCTGTAG | |
| | AGATAGGGAATGGATGCTTTGAAACCAAACACAAGTGCAACCAGACCTGCC | |
| | TCGACAGAATAGCTGCTGGTACCTTTGATGCAGGAGAATTTTCTCTCCCCAC | |
| | CTTTGATTCACTGAATATTACTGCTGCATCTTTAAATGACGACGGATTGGAC | |
| | AATCATACTATACTGCTTTACTACTCAACTGCTGCCTCCAGTTTGGCTGTAAC | |
| | ACTGATGATAGCTATCTTTGTTGTTTATATGGTCTCCAGAGACAATGTTTCTT | |
| | GCTCCATTTGTCTATAAGGAAAGTTAAGCCCTGTATTTTCCTTTATTGTAGTG | |
| | CTTGTTTGCTTGTTGTCATTACAAAGAAACGTTATTGAAAAATTGATGA | |

In some embodiments, the vaccine compositions described herein comprises a sequence that is at least to 100%, 98%, 95%, 90%, 85%, 80%, 75%, or 50% identical to the aforementioned sequences (i.e., the sequences within Table 4).

### Molecular Adjuvants and T Cell Enhancements

In certain embodiments, the vaccine composition comprises a molecular adjuvant and/or one or more T-cell enhancement compositions. The adjuvant and/or enhancement compositions may help improve the immunogenicity and/or long-term memory of the vaccine composition. Non-limiting examples of molecular adjuvants include CpG, such as a CpG polymer, and flagellin.

In some embodiments, the vaccine composition comprises a T cell attracting chemokine. The T cell attracting chemokine helps pull the T cells from the circulation to the appropriate tissues, e.g., the lungs, heart, kidney, and brain. Non-limiting examples of T cell attracting chemokines include CCL5, CXCL9, CXCL10, CXCL11, CCL25, CCL28, CXCL14, CXCL17, or a combination thereof.

In some embodiments, the vaccine composition comprises a composition that promotes T-cell proliferation. Non-limiting examples of compositions that promote T-cell proliferation include IL-7, IL-15, IL-2, or a combination thereof.

In some embodiments, the vaccine composition comprises a composition that promotes T-cell homing in the lungs. Non-limiting examples of compositions that promote T-cell homing include CCL25, CCL28, CXCL14, CXCL17, or a combination thereof.

In certain embodiments, the molecular adjuvant and/or the T cell attracting chemokine and/or the composition that promotes T cell proliferation are delivered with a separate antigen delivery system from the large sequences. In other embodiments, the molecular adjuvant and/or the T cell attracting chemokine and/or the composition that promotes T cell proliferation are delivered by the same antigen delivery system as the large sequences.

In some embodiments, one or more linkers may be used between the T cell attracting chemokines and/or the compositions that promote T cell proliferation. The linkers may allow for cleavage of the separate molecules (e.g., chemokine). For example, in some embodiments, a linker is positioned between IL-7 (or IL-2) and CCL5, CXCL9, CXCL10, CXCL11, CCL25, CCL28, CXCL14, CXCL17, etc. In some embodiments, a linker is positioned between IL-15 and CCL5, CXCL9, CXCL10, CXCL11, CCL25, CCL28, CXCL14, CXCL17, etc. In some embodiments, a linker is positioned between the antigen or large sequence and another composition, e.g., IL-15, IL-7, CCL5, CXCL9, CXCL10, CXCL11, CCL25, CCL28, CXCL14, CXCL17, etc. A non-limiting example of a linker is T2A, E2A, P2A, or the like. The composition may feature a different linker between each open reading frame.

Table 5 shows non-limiting examples of T-cell enhancements that may be used to create a vaccine composition described herein.

| T-cell | | SEQ |
|---|---|---|
| enhancement | Sequence | ID NO: |
| CXCL11 | ATGAACAGGAAGGTGACCGCCATCGCCCTGGCCGCCATCATCTGGGCCA | 54 |
| | CCGCCGCCCAGGGCTTCCTGATGTTCAAGCAGGGCAGGTGCCTGTGCAT | |
| | CGGCCCCGGCATGAAGGCCGTGAAGATGGCCGAGATCGAGAAGGCCAG | |
| | CGTGATCTACCCCAGCAACGGCTGCGACAAGGTGGAGGTGATCGTGACC | |
| | ATGAAGGCCCACAAGAGGCAGAGGTGCCTGGACCCCAGGAGCAAGCAGG | |
| | CCAGGCTGATCATGCAGGCCATCGAGAAGAAGAACTTCCTGAGGAGGCA | |
| | GAACATGTGA | |
| CCL5 | ATGAAGGTCTCCGCGGCAGCCCTCGCTGTCATCCTCATTGCTACTGCCCT | 55 |
| | CTGCGCTCCTGCATCTGCCTCCCCATATTCCTCGGACACCACACCCTGCT | |
| | GCTTTGCCTACATTGCCCGCCCACTGCCCCGTGCCCACATCAAGGAGTAT | |
| | TTCTACACCAGTGGCAAGTGCTCCAACCCAGCAGTCGTCCACAGGTCAAG | |
| | GATGCCAAAGAGAGAGGGACAGCAAGTCTGGCAGGATTTCCTGTATGACT | |
| | CCCGGCTGAACAAGGGCAAGCTTTGTCACCCGAAAGAACCGCCAAGTGT | |
| | GTGCCAACCCAGAGAAGAAATGGGTTCGGGAGTACATCAACTCTTTGGAG | |
| | ATGAGCTAGGATGGAGAGTCCTTGAACCTGAACTTACACAAATTTGCCTGT | |
| | TTCTGCTTGCTCTTGTCCTAGCTTGGGAGGCTTCCCCTCACTATCCTACCC | |
| | CACCCGCTCCTTGA | |
| CXCL9 | ATGAAGAAAAGTGGTGTTCTTCCTCTTGGGCATCATCTTGCTGGTTCTG | 56 |
| | ATTGGAGTGCAAGGAACCCCAGTAGTGAGAAAGGGTCGCTGTTCCTGCAT | |
| | CAGCACCAACCAAGGGACTATCCACCTACAATCCTTGAAAGACCTTAAACA | |
| | ATTTGCCCCAAGCCCTTCCTGCGAGAAAATTGAAATCATTGCTACACTGAA | |
| | GAATGGAGTTCAAACATGTCTAAACCCAGATTCAGCAGATGTGAAGGAACT | |
| | GATTAAAAAGTGGGAGAAACAGGTCAGCCAAAAGAAAAAGCAAAAGAATG | |
| | GGAAAAAACATCAAAAAAAGAAAGTTCTGAAAGTTCGAAAATCTCAACGTT | |
| | CTCGTCAAAAGAAGACTACATAA | |
| CXCL10 | ATGAATCAAACTGCCATTCTGATTTGCTGCCTTATCTTTCTGACTCTAAGTG | 57 |
| | GCATTCAAGGAGTACCTCTCTCTAGAACTGTACGCTGTACCTGCATCAGCA | |
| | TTAGTAATCAACCTGTTAATCCAAGGTCTTTAGAAAAACTTGAAATTATTCC | |
| | TGCAAGCCAATTTTGTCCACGTGTTGAGATCATTGCTACAATGAAAAAGAA | |
| | GGGTGAGAAGAGATGTCTGAATCCAGAATCGAAGGCCATCAAGAATTTAC | |
| | TGAAAGCAGTTAGCAAGGAAAGGTCTAAAAGATCTCCTTAA | |
| CXCL14 | ATGAGGCTCCTGGCGGCCGCGCTGCTCCTGCTGCTGCTGGCGCTGTACA | 58 |
| | CCGCGCGTGTGGACGGGTCCAAATGCAAGTGCTCCCGGAAGGGACCCAA | |
| | GATCCGCTACAGCGACGTGAAGAAGCTGGAAATGAAGCCAAAGTACCCGC | |
| | ACTGCGAGGAGAAGATGGTTATCATCACCACCAAGAGCGTGTCCAGGTAC | |
| | CGAGGTCAGGAGCACTGCCTGCACCCCAAGCTGCAGAGCACCAAGCGCT | |
| | TCATCAAGTGGTACAACGCCTGGAACGAGAAGCGCAGGGTCTACGAAGAA | |
| | TAG | |
| CXCL17 | ATGAAAGTTCTAATCTCTTCCCTCCTCCTGTTGCTGCCACTAATGCTGATG | 59 |
| | TCCATGGTCTCTAGCAGCCTGAATCCAGGGGTCGCCAGAGGCCACAGGG | |
| | ACCGAGGCCAGGCTTCTAGGAGATGGCTCCAGGAAGGCGGCCAAGAATG | |
| | TGAGTGCAAAGATTGGTTCCTGAGAGCCCCGAGAAGAAAATTCATGACAG | |
| | TGTCTGGGCTGCCAAAGAAGCAGTGCCCCTGTGATCATTTCAAGGGCAAT | |
| | GTGAAGAAAACAAGACACCAAAGGCACCACAGAAAGCCAAACAAGCATTC | |
| | CAGAGCCTGCCAGCAATTTCTCAAACAATGTCAGCTAAGAAGCTTTGCTCT | |
| | GCCTTTGTAG | |
| CCL25 | ATGAACCTGTGGCTCCTGGCCTGCCTGGTGGCCGGCTTCCTGGGAGCCT | 60 |
| | GGGCCCCCGCTGTCCACACCCAAGGTGTCTTTGAGGACTGCTGCCTGGC | |
| | CTACCACTACCCCATTGGGTGGGCTGTGCTCCGGCGCGCCTGGACTTAC | |
| | CGGATCCAGGAGGTGAGCGGGAGCTGCAATCTGCCTGCTGCGATATTCTA | |
| | CCTCCCCAAGAGACACAGGAAGGTGTGTGGGAACCCCAAAAGCAGGGAG | |
| | GTGCAGAGAGCCATGAAGCTCCTGGATGCTCGAAATAAGGTTTTTGCAAA | |
| | GCTCCACCACAACACGCAGACCTTCCAAGCAGGCCCTCATGCTGTAAAGA | |
| | AGTTGAGTTCTGGAAACTCCAAGTTATCATCGTCCAAGTTTAGCAATCCCA | |
| | TCAGCAGCAGTAAGAGGAATGTCTCCCTCCTGATATCAGCTAATTCAGGAC | |
| | TGTGA | |
| CCL28 | ATGCAGCAGAGAGGACTCGCCATCGTGGCCTTGGCTGTCTGTGCGGCCC | 61 |
| | TACATGCCTCAGAAGCCATACTTCCCATTGCCTCCAGCTGTTGCACGGAG | |
| | GTTTCACATCATATTTCCAGAAGGCTCCTGGAAAGAGTGAATATGTGTCGC | |
| | ATCCAGAGAGCTGATGGGGATTGTGACTTGGCTGCTGTCATCCTTCATGT | |
| | CAAGCGCAGAAGAATCTGTGTCAGCCCGCACAACCATACTGTTAAGCAGT | |
| | GGATGAAAGTGCAAGCTGCCAAGAAAAATGGTAAAGGAAATGTTTGCCAC | |
| | AGGAAGAAACACCATGGCAAGAGGAACAGTAACAGGGCACATCAGGGGA | |
| | AACACGAAACATACGGCCATAAAACTCCTTATTAG | |
| IL-7 | ATGTTCCACGTGAGCTTCAGGTACATCTTCGGCATCCCCCCCCTGATCCT | 62 |
| | GGTGCTGCTGCCCGTGACCAGCAGCGAGTGCCACATCAAGGACAAGGAG | |
| | GGCAAGGCCTACGAGAGCGTGCTGATGATCAGCATCGACGAGCTGGACA | |
| | AGATGACCGGCACCGACAGCAACTGCCCCAACAACGAGCCCAACTTCTTC | |
| | AGGAAGCACGTGTGCGACGACACCAAGGAGGCCGCCTTCCTGAACAGGG | |
| | CCGCCAGGAAGCTGAAGCAGTTCCTGAAGATGAACATCAGCGAGGAGTTC | |
| | AACGTGCACCTGCTGACCGTGAGCCAGGGCACCCAGACCCTGGTGAACT | |
| | GCACCAGCAAGGAGGAGAAGAACGTGAAGGAGCAGAAGAAGAACGACGC | |
| | CTGCTTCCTGAAGAGGCTGCTGAGGGAGATCAAGACCTGCTGGAACAAGA | |
| | TCCTGAAGGGCAGCATCTGA | |
| IL-15 | ATGAGAATTTCGAAACCACATTTGAGAAGTATTTCCATCCAGTGCTACTTGT | 63 |
| | GTTTACTTCTAAACAGTCATTTTCTAACTGAAGCTGGCATTCATGTCTTCAT | |
| | TTTGGGCTGTTTCAGTGCAGGGCTTCCTAAAACAGAAGCCAACTGGGTGA | |
| | ATGTAATAAGTGATTTGAAAAAAATTGAAGATCTTATTCAATCTATGCATATT | |
| | GATGCTACTTTATATACGGAAAGTGATGTTCACCCCAGTTGCAAAGTAACA | |
| | GCAATGAAGTGCTTTCTCTTGGAGTTACAAGTTATTTCACTTGAGTCCGGA | |
| | GATGCAAGTATTCATGATACAGTAGAAAATCTGATCATCCTAGCAAACAAC | |
| | AGTTTGTCTTCTAATGGGAATGTAACAGAATCTGGATGCAAAGAATGTGAG | |
| | GAACTGGAGGAAAAAAATATTAAAGAATTTTTGCAGAGTTTTGTACATATTG | |
| | TCCAAATGTTCATCAACACTTCTTGA | |
| IL-2 | ATGTACAGGATGCAACTCCTGTCTTGCATTGCACTAAGTCTTGCACTTGTC | 64 |
| | ACAAACAGTGCACCTACTTCAAGTTCTACAAAGAAAACACAGCTACAACTG | |
| | GAGCATTTACTGCTGGATTTACAGATGATTTTGAATGGAATTAATAATTACA | |
| | AGAATCCCAAACTCACCAGGATGCTCACATTTAAGTTTTACATGCCCAAGA | |
| | AGGCCACAGAACTGAAACATCTTCAGTGTCTAGAAGAAGAACTCAAACCTC | |
| | TGGAGGAAGTGCTAAATTTAGCTCAAAGCAAAAACTTTCACTTAAGACCCA | |
| | GGGACTTAATCAGCAATATCAACGTAATAGTTCTGGAACTAAAGGGATCTG | |
| | AAACAACATTCATGTGTGAATATGCTGATGAGACAGCAACCATTGTAGAAT | |
| | TTCTGAACAGATGGATTACCTTTTGTCAAAGCATCATCTCAACACTGACTTG | |
| | A | |

In some embodiments, the vaccine compositions described herein comprises T cell attracting chemokine and/or the composition that promotes T cell proliferation that are encoded by a sequence that is at least 100%, 98%, 95%, 90%, 85%, 80%, 75%, or 50% identical to the aforementioned sequences.

In preferred embodiments, the T-cell enhancement compositions described herein (e.g., CXCL9, CXCL10, IL-7, IL-2) may be integrated into a separate delivery system from the vaccine compositions. In some embodiments, the T-cell enhancement compositions described herein (e.g., CXCL9, CXCL10, IL-7, IL-2) may be integrated into the same delivery system as the vaccine compositions.

In certain embodiments, the vaccine composition comprises a tag. For example, in some embodiments, the vaccine composition comprises a His tag. The present invention is not limited to a His tag and includes other tags such as those known to one of ordinary skill in the art, such as a fluorescent tag (e.g., GFP, YFP, etc.), etc.

### Delivery System

The present invention also features vaccine compositions in the form of a delivery system (e.g., an antigen delivery system). Any appropriate antigen delivery system may be considered for delivery of the antigens described herein. The present invention is not limited to the antigen delivery systems described herein.

In certain embodiments, the antigen delivery system is for targeted delivery of the vaccine composition, e.g., for targeting to the tissues of the body where the virus replicates.

In certain embodiments, the antigen delivery system comprises adenoviruses such as but not limited to Ad5, Ad26, Ad35, etc., as well as carriers such as lipid nanoparticles, polymers, peptides, etc. In other embodiments, the antigen delivery system comprises a vesicular stomatitis virus (VSV) vector.

The present invention is not limited to adenovirus vector-based antigen delivery systems. In certain embodiments, the antigen delivery system comprises an adeno-associated virus vector-based antigen delivery system, such as but not limited to the adeno-associated virus vector type 9 (AAV9 serotype), AAV type 8 (AAV8 serotype), etc. In certain embodiments, the adeno-associated virus vectors used are tropic, e.g., tropic to lungs, brain, heart, and kidney, e.g., the tissues of the body that express ACE2 receptors. For example, AAV9 is known to be neurotropic, which would help the vaccine composition to be expressed in the brain.

In the antigen delivery system, the one or more antigens are operatively linked to a promoter. In certain embodiments, the one or more large sequences are operatively linked to a generic promoter. For example, in certain embodiments, the one or more antigens are operatively linked to a CMV promoter. In certain embodiments, the one or more antigens are operatively linked to a CAG, EFIA, EFS, CBh, SFFV, MSCV, mPGK, hPGK, SV40, UBC, or another appropriate promoter.

In some embodiments, the one or more antigens are operatively linked to a tissue-specific promoter (e.g., a lung-specific promoter). For example, the antigen may be operatively linked to a SpB promoter or a CD144 promoter.

As discussed, in certain embodiments, the vaccine composition comprises a molecular adjuvant. In certain embodiments, the molecular adjuvant is operatively linked to a generic promoter, e.g., as described above. In certain embodiments, the molecular adjuvant is operatively linked to a tissue-specific promoter, e.g., a lung-specific promoter, e.g., SpB or CD144.

As discussed, in certain embodiments, the vaccine composition comprises a T cell attracting chemokine. In certain embodiments, the T cell attracting chemokine is operatively linked to a generic promoter, e.g., as described above. In certain embodiments, the T cell attracting chemokine is operatively linked to a tissue-specific promoter, e.g., a lung-specific promoter, e.g., SpB or CD144.

As discussed, in certain embodiments, the vaccine composition comprises a composition for promoting T cell proliferation. In certain embodiments, the composition for promoting T cell proliferation is operatively linked to a generic promoter, e.g., as described above. In certain embodiments, the composition for promoting T cell proliferation is operatively linked to a tissue-specific promoter, e.g., a lung-specific promoter, e.g., SpB or CD144.

In some embodiments, the antigens (e.g., the Coronavirus antigens and/or the Influenza antigen; e.g., large sequences) are operatively linked to a generic promoter. The generic promoter may be a CMV or a CAG promoter. In certain embodiments, the antigens (e.g., the Coronavirus antigens and/or the Influenza antigen; e.g., large sequences) are operatively linked to a tissue-specific promoter, e.g., a lung-specific promoter, e.g., SpB or CD144.

Table 6 shows non-limiting examples of a portion of a promoter that may be used in accordance with the present invention.

| | | SEQ |
|---|---|---|
| Promoter | Sequence | ID NO: |
| CMV | TAGTTATTAATAGTAATCAATTACGGGGTCATTAGTTCATAGCCCATATATGGAGT | 17 |
| | TCCGCGTTACATAACTTACGGTAAATGGCCCGCCTGGCTGACCGCCCAACGACC | |
| | CCCGCCCATTGACGTCAATAATGACGTATGTTCCCATAGTAACGCCAATAGGGA | |
| | CTTTCCATTGACGTCAATGGGTGGAGTATTTACGGTAAACTGCCCACTTGGCAGT | |
| | ACATCAAGTGTATCATATGCCAAGTACGCCCCCTATTGACGTCAATGACGGTAAA | |
| | TGGCCCGCCTGGCATTATGCCCAGTACATGACCTTATGGGACTTTCCTACTTGG | |
| | CAGTACATCTACGTATTAGTCATCGCTATTACCATGGTGATGCGGTTTTGGCAGT | |
| | ACATCAATGGGCGTGGATAGCGGTTTGACTCACGGGGATTTCCAAGTCTCCACC | |
| | CCATTGACGTCAATGGGAGTTTGTTTTGGCACCAAAATCAACGGGACTTTCCAAA | |
| | ATGTCGTAACAACTCCGCCCCATTGACGCAAATGGGCGGTAGGCGTGTACGGTG | |
| | GGAGGTCTATATAAGCAGAGCTGGTTTAGTGAACCGTCAGATC | |
| CAG | CTCGACATTGATTATTGACTAGTTATTAATAGTAATCAATTACGGGGTCATTAGTT | 167 |
| | CATAGCCCATATATGGAGTTCCGCGTTACATAACTTACGGTAAATGGCCCGCCTG | |
| | GCTGACCGCCCAACGACCCCCGCCCATTGACGTCAATAATGACGTATGTTCCCA | |
| | TAGTAACGCCAATAGGGACTTTCCATTGACGTCAATGGGTGGAGTATTTACGGTA | |
| | AACTGCCCACTTGGCAGTACATCAAGTGTATCATATGCCAAGTACGCCCCCTATT | |
| | GACGTCAATGACGGTAAATGGCCCGCCTGGCATTATGCCCAGTACATGACCTTA | |
| | TGGGACTTTCCTACTTGGCAGTACATCTACGTATTAGTCATCGCTATTACCATGG | |
| | TCGAGGTGAGCCCCACGTTCTGCTTCACTCTCCCCATCTCCCCCCCCTCCCCAC | |
| | CCCCAATTTTGTATTTATTTATTTTTTAATTATTTTGTGCAGCGATGGGGGCGGGG | |
| | GGGGGGGGGGGGCGCGCGCCAGGCGGGGCGGGGCGGGGCGAGGGGCGGGG | |
| | CGGGGCGAGGCGGAGAGGTGCGGCGGCAGCCAATCAGAGCGGCGCGCTCC | |
| | GAAAGTTTCCTTTTATGGCGAGGCGGCGGCGGCGGCGGCCCTATAAAAAGCGA | |
| | AGCGCGCGGCGGGCGGGAGTCGCTGCGCGCTGCCTTCGCCCCGTGCCCCGCT | |
| | CCGCCGCCGCCTCGCGCCGCCCGCCCCGGCTCTGACTGACCGCGTTACTCCCA | |
| | CAGGTGAGCGGGCGGGACGGCCCTTCTCCTCCGGGCTGTAATTAGCGCTTGGT | |
| | TTAATGACGGCTTGTTTCTTTTCTGTGGCTGCGTGAAAGCCTTGAGGGGCTCCG | |
| | GGAGGGCCCTTTGTGCGGGGGGAGCGGCTCGGGGGGTGCGTGCGTGTGTGTG | |
| | TGCGTGGGGAGCGCCGCGTGCGGCTCCGCGCTGCCCGGCGGCTGTGAGCGCT | |
| | GCGGGCGCGGCGCGGGGCTTTGTGCGCTCCGCAGTGTGCGCGAGGGGAGCG | |
| | CGGCCGGGGGCGGTGCCCCGCGGTGCGGGGGGGGCTGCGAGGGGAACAAAG | |
| | GCTGCGTGCGGGGTGTGTGCGTGGGGGGGTGAGCAGGGGGTGTGGGCGCGT | |
| | CGGTCGGGCTGCAACCCCCCCTGCACCCCCCTCCCCGAGTTGCTGAGCACGGC | |
| | CCGGCTTCGGGTGCGGGGCTCCGTACGGGGCGTGGCGCGGGGCTCGCCGTGC | |
| | CGGGCGGGGGGTGGCGGCAGGTGGGGGTGCCGGGCGGGGCGGGGCCGCCT | |
| | CGGGCCGGGGAGGGCTCGGGGGAGGGGCGCGGCGGCCCCCGGAGCGCCGG | |
| | CGGCTGTCGAGGCGCGGCGAGCCGCAGCCATTGCCTTTTATGGTAATCGTGCG | |
| | AGAGGGCGCAGGGACTTCCTTTGTCCCAAATCTGTGCGGAGCCGAAATCTGGG | |
| | AGGCGCCGCCGCACCCCCTCTAGCGGGCGCGGGGCGAAGCGGTGCGGCGCC | |
| | GGCAGGAAGGAAATGGGCGGGGAGGGCCTTCGTGCGTCGCCGCGCCGCCGTC | |
| | CCCTTCTCCCTCTCCAGCCTCGGGGCTGTCCGCGGGGGGACGGCTGCCTTCGG | |
| | GGGGGACGGGGCAGGGCGGGGTTCGGCTTCTGGCGTGTGACCGGCGGCTCTA | |
| | GAGCCTCTGCTAACCATGTTCATGCCTTCTTCTTTTTCCTACAGCTCCTGGGCAA | |
| | CGTGCTGGTTATTGTGCTGTCTCATCATTTTGGCAAAGAATTG | |
| SP-B | GTATAGGGCTGTCTGGGAGCCACTCCAGGGCCACAGAAATCTTGTCTCTGACTC | 19 |
| | AGGGTATTTTGTTTTCTGTTTTGTGTAAATGCTCTTCTGACTAATGCAAACCATGT | |
| | GTCCATAGAACCAGAAGATTTTTCCAGGGGAAAAGGTAAGGAGGTGGTGAGAGT | |
| | GTCCTGGGTCTGCCCTTCCAGGGCTTGCCCTGGGTTAAGAGCCAGGCAGGAAG | |
| | CTCTCAAGAGCATTGCTCAAGAGTAGAGGGGGCCTGGGAGGCCCAGGGAGGG | |
| | GATGGGAGGGGAACACCCAGGCTGCCCCCAACCAGATGCCCTCCACCCTCCTC | |
| | AACCTCCCTCCCACGGCCTGGAGAGGTGGGACCAGGTATGGAGGCTTGAGAGC | |
| | CCCTGGTTGGAGGAAGCCACAAGTCCAGGAACATGGGAGTCTGGGCAGGGGGC | |
| | AAAGGAGGCAGGAACAGGCCATCAGCCAGGACAGGTGGTAAGGCAGGCAGGA | |
| | GTGTTCCTGCTGGGAAAAGGTGGGATCAAGCACCTGGAGGGCTCTTCAGAGCA | |
| | AAGACAAACACTGAGGTCGCTGCCACTCCTACAGAGCCCCCACGCCCCGCCCA | |
| | GCTATAAGGGGCCATGCACCAAGCAGGGTACCCAGGCTGCAGAGGTGCC | |
| CD144 | CATCCATGCCCATGGCCTCAGATGCCAGCCATAAGCTGTTGGGTTCCAAACCTC | 20 |
| | GACTCCAGGCTGGACTCACCCCTGTCTCCCCCACCAGCCTGACACCTCCACCTG | |
| | GGTATCTAACGAGCATCTCAAACTCAACCTGCCTGAGACAGAGGAATCACTATCC | |
| | CCTCCTCCTCCAAAAATATCCTTCCATCACACTCCCCATCTTGTGCTCTGATTTAC | |
| | TAAACGGCCCTGGGCCCTCTCTTTCTCAGGGTCTCTGCTTGCCCAGCTATATAAT | |
| | AAAACAAGTTTGGGACTTCCCAACCATTCACCCATGGAAAAACAGAAGCAACTCT | |
| | TCAAAGGACAGATTCCCAGGATCTGCCCTGGGAGATTCCAAATCAGTTGATCTG | |
| | GGGTGAGCCCAGTCCTCTGTAGTTTTTAGAAGCTCCTCCTATGTCTCTCCTGGTC | |
| | AGCAGAATCTTGGCCCCTCCCTTCCCCCCAGCCTCTTGGTTCTTCTGGGCTCTG | |
| | ATCCAGCCTCAGCGTCACTGTCTTCCACGCCCCTCTTTGATTCTCGTTTATGTCA | |
| | AAAGCCTTGTGAGGATGAGGCTGTGATTATCCCCATTTTACAGATGAGGAAACTG | |
| | TGGCTCCAGGATGACACAACTGGCCAGAGGTCACATCAGAAGCAGAGCTGGGT | |
| | CACTTGACTCCACCCAATATCCCTAAATGCAAACATCCCCTACAGACCGAGGCTG | |
| | GCACCTTAGAGCTGGAGTCCATGCCCGCTCTGACCAGGAGAAGCCAACCTGGT | |
| | CCTCCAGAGCCAAGAGCTTCTGTCCCTTTCCCATCTCCTGAAGCCTCCCTGTCA | |
| | CCTTTAAAGTCCATTCCCACAAAGACATCATGGGATCACCACAGAAAATCAAGCT | |
| | CTGGGGCTAGGCTGACCCCAGCTAGATTTTTGGCTCTTTTATACCCCAGCTGGG | |
| | TGGACAAGCACCTTAAACCCGCTGAGCCTCAGCTTCCCGGGCTATAAAATGGGG | |
| | GTGATGACACCTGCCTGTAGCATTCCAAGGAGGGTTAAATGTGATGCTGCAGCC | |
| | AAGGGTCCCCACAGCCAGGCTCTTTGCAGGTGCTGGGTTCAGAGTCCCAGAGC | |
| | TGAGGCCGGGAGTAGGGGTTCAAGTGGGGTGCCCCAGGCAGGGTCCAGTGCC | |
| | AGCCCTCTGTGGAGACAGCCATCCGGGGCCGAGGCAGCCGCCCACCGCAGGG | |
| | CCTGCCTATCTGCAGCCAGCCCAGCCCTCACAAAGGAACAATAACAGGAAACCA | |
| | TCCCAGGGGGAAGTGGGCCAGGGCCAGCTGGAAAACCTGAAGGGGAGGCAGC | |
| | CAGGCCTCCCTCGCCAGCGGGGTGTGGCTCCCCTCCAAAGACGGTCGGCTGAC | |
| | AGGCTCCACAGAGCTCCACTCACGCTCAGCCCTGGACGGACAGGCAGTCCAAC | |
| | GGAACAGAAACATCCCTCAGCCCACAGGCACGGTGAGTGGGGGCTCCCACACT | |
| | CCCCTCCACCCCAAACCCGCCACCCTGCGCCCAAGATGGGAGGGTCCTCAGCT | |
| | TCCCCATCTGTAGAATGGGCATCGTCCCACTCCCATGACAGAGAGGCTCC | |

In some embodiments, the vaccine compositions described herein comprises promoters that are encoded by a sequence that is at least 100%, 98%, 95%, 90%, 85%, 80%, 75%, or 50% identical to the aforementioned sequences.

In certain embodiments, the T cell attracting chemokine and the composition that promotes T cell proliferation are driven by the same promoter (e.g., the T cell attracting chemokine and the composition that promotes T cell proliferation are synthesized as a peptide). In certain embodiments, the T cell attracting chemokine and the composition that promotes T cell proliferation are driven by different promoters. In certain embodiments, the antigen, the T cell attracting chemokine, and the composition that promotes T cell proliferation are driven by the same promoter. In certain embodiments, the antigen, the T cell attracting chemokine, and the composition that promotes T cell proliferation are driven by the different promoters. In certain embodiments, the T cell attracting chemokine and the composition that promotes T cell proliferation are driven by the same promoter, and the one or more large sequences are driven by a different promoter.

In some embodiments, the Influenza antigen and the Coronavirus antigen(s) are driven by the same promoter (e.g., the Influenza antigen and the Coronavirus antigen(s) are synthesized as a peptide). In other embodiments, the Influenza antigen and the Coronavirus antigen(s) are driven by different promoters.

Table 7: Shows non-limiting examples of linkers that may be used in accordance with the present invention.

| | | SEQ |
|---|---|---|
| Linker | SEQUENCE | ID NO: |
| T2A Linker | GGAAGCGGAGAGGGCAGGGGAAGTCTTCTAACATGCGGGGACGTGG | 65 |
| | AGGAAAATCCCGGCCCC | |
| E2A Linker | GGAAGCGGACAGTGTACTAATTATGCTCTCTTGAAATTGGCTGGAGAT | 66 |
| | GTTGAGAGCAACCCAGGTCCC | |
| P2A Linker | GGAAGCGGAGCCACGAACTTCTCTCTGTTAAAGCAAGCAGGAGATGT | 67 |
| | TGAAGAAAACCCCGGGCCT | |
| | GCCGCCTAC | — |
| | GGCCCCGGCCCCGGC | 68 |
| 6-His Tag | CATCACCATCACCATCAC | 181 |

The present invention includes mRNA sequences encoding any of the vaccine compositions or portions thereof herein, e.g., a molecular adjuvant, a T cell enhancement, etc. The present invention also includes modified mRNA sequences encoding any of the vaccine compositions or portions thereof herein. The present invention also includes DNA sequence encoding any of the vaccine compositions or portions thereof herein.

In some embodiments, the mRNA sequence encodes at least one antigen. In other embodiments, the mRNA sequence encodes at least two (preferably different) antigens. In certain embodiments, the mRNA sequence encodes at least three (preferably different) antigens. In other embodiments, the mRNA sequence encodes at least four (preferably different) antigens.

The antigens encoded by the aforementioned mRNA sequences may be selected from the described Coronavirus antigens, Influenza antigens, or a combination of both. For example, the mRNA sequence may encode one or more Coronavirus antigens (e.g., a Spike protein), and a separate mRNA sequence may encode one or more Influenza antigens (e.g., an HA protein).

Thus, in some embodiments, the mRNA sequence may encode a Coronavirus Spike protein, and a separate mRNA sequence may encode an Influenza HA protein. In some embodiments, the mRNA sequence may encode a Coronavirus NSP2 protein, and a separate mRNA sequence may encode an Influenza HA protein. In some embodiments, the mRNA sequence may encode a Coronavirus NSP14 protein, and a separate mRNA sequence may encode an Influenza HA protein. In some embodiments, the mRNA sequence may encode a Coronavirus Nucleoprotein, and a separate mRNA sequence may encode an Influenza HA protein.

In some embodiments, the mRNA sequence may encode a Coronavirus NSP2 protein, a Coronavirus NSP14 protein, and a Coronavirus Nucleoprotein. Additionally, a separate mRNA sequence may encode an Influenza HA protein. In other embodiments, the mRNA sequence may encode a Coronavirus NSP2 protein, a Coronavirus NSP14 protein, a Coronavirus Nucleoprotein, and a Coronavirus Spike protein, and a separate mRNA sequence encodes an Influenza HA protein.

Alternatively, the mRNA sequence may encode both the Coronavirus antigens (e.g., a Spike protein) and the Influenza antigens (e.g., an HA protein).

In certain embodiments, nucleic acids of a vaccine composition herein are chemically modified. In some embodiments, the nucleic acids of a vaccine composition therein are unmodified. In some embodiments, all or a portion of the uracil in the open reading frame has a chemical modification. In some embodiments, a chemical modification is in the 5-position of the uracil. In some embodiments, a chemical modification is an N1-methyl pseudouridine. In some embodiments, all or a portion of the uracil in the open reading frame has an N1-methyl pseudouridine in the 5-position of the uracil.

In certain embodiments, an open reading frame of a vaccine composition herein encodes one antigen or epitope. In some embodiments, an open reading frame of a vaccine composition herein encodes two or more antigens or epitopes. In some embodiments, an open reading frame of a vaccine composition herein encodes three antigens or epitopes. In some embodiments, an open reading frame of a vaccine composition herein encodes five or more antigens or epitopes. In some embodiments, an open reading frame of a vaccine composition herein encodes ten or more antigens or epitopes. In some embodiments, an open reading frame of a vaccine composition herein encodes 50 or more antigens or epitopes.

The mRNAs may further comprise a 5′ untranslated region (UTR) and a 3′ UTR. In some embodiments, the mRNAs further comprise a 3′ poly(A) tail and/or a 5′ cap or cap analog.

For example, the present invention may further feature a composition comprising one (or more) ribonucleic acids (mRNAs) comprising an open reading frame encoding at least a portion of a Coronavirus protein selected from a group comprising, consisting essentially or consisting of: an NSP2 protein, an NSP14 protein, a Nucleoprotein protein and a Spike protein. In some embodiments, the composition comprising two (or more) ribonucleic acids (mRNAs) comprising an open reading frame encoding at least a portion of a Coronavirus protein selected from a group comprising, consisting essentially or consisting of: an NSP2 protein, an NSP14 protein, a Nucleoprotein and a Spike protein. In some embodiments, the composition comprising three (or more) ribonucleic acids (mRNAs) comprising an open reading frame encoding at least a portion of a Coronavirus protein selected from a group comprising, consisting essentially or consisting of: an NSP2 protein, an NSP14 protein, a Nucleoprotein and a Spike protein. The one, two, or three mRNAs may be formulated in a lipid nanoparticle.

Alternatively, the composition may comprise one (or more) mRNAs comprising an open reading frame encoding an entire Coronavirus protein selected from a group comprising, consisting essentially, or consisting of: an NSP2 protein, an NSP14 protein, a Nucleoprotein protein, and a Spike protein. In other embodiments, the composition may comprise two (or more) mRNAs comprising an open reading frame encoding an entire Coronavirus protein selected from a group comprising, consisting essentially, or consisting of: an NSP2 protein, an NSP14 protein, a Nucleoprotein protein and a Spike protein. In other embodiments, the composition may comprise three (or more) mRNAs comprising an open reading frame encoding an entire Coronavirus protein selected from a group comprising, consisting essentially, or consisting of: an NSP2 protein, an NSP14 protein, a Nucleoprotein protein and a Spike protein.

The composition may further comprise an mRNA comprising an open reading frame encoding an Influenza protein (e.g., an HA protein) or portion thereof formulated in a lipid nanoparticle. For example, the composition may comprise two (or more) mRNAs comprising an open reading frame encoding at least a portion of a Coronavirus protein selected from a group comprising, consisting essentially or consisting of: an NSP2 protein, an NSP14 protein, a Nucleoprotein protein and a Spike protein and a portion of a Influenza protein (e.g., an HA protein). In other embodiments, the composition may comprise three (or more) mRNAs comprising an open reading frame encoding at least a portion of a Coronavirus protein selected from a group comprising, consisting essentially or consisting of: an NSP2 protein, an NSP14 protein, a Nucleoprotein, and a Spike protein and a portion of an Influenza protein (e.g., an HA protein).

Alternatively, the composition may comprise two (or more) mRNAs comprising an open reading frame encoding an entire Coronavirus protein selected from a group comprising, consisting essentially, or consisting of: an NSP2 protein, an NSP14 protein, a Nucleoprotein, a Spike protein, and an entire Influenza protein (e.g., an HA protein). In some embodiments, the composition may comprise three (or more) mRNAs comprising an open reading frame encoding an entire Coronavirus protein selected from a group comprising, consisting essentially, or consisting of: an NSP2 protein, an NSP14 protein, a Nucleoprotein protein, a Spike protein; an entire Influenza protein (e.g., an HA protein). The two or three (or more) mRNAs are formulated in a lipid nanoparticle.

In some embodiments, the mRNAs encoding the Coronavirus proteins and mRNA encoding the Influenza proteins are formulated within the same lipid nanoparticle. In other embodiments, the mRNAs encoding the Coronavirus proteins and mRNA encoding the Influenza proteins are formulated within different lipid nanoparticles.

### Methods

As previously discussed, the compositions described herein, e.g., the antigens, the vaccine compositions, the antigen delivery systems, the chemokines, the adjuvants, etc., may be used to prevent a Coronavirus and/or Influenza infection in a subject. In some embodiments, the compositions described herein, e.g., the antigens, the vaccine compositions, the antigen delivery systems, the chemokines, the adjuvants, etc., may be used to prevent a Coronavirus and/or Influenza infection prophylactically in a subject. In some embodiments, the compositions described herein, e.g., the antigens, the vaccine compositions, the antigen delivery systems, the chemokines, the adjuvants, etc., may elicit an immune response in a subject. In some embodiments, the compositions described herein, e.g., the antigens, the vaccine compositions, the antigen delivery systems, the chemokines, the adjuvants, etc., may prolong an immune response induced by the hybrid vaccine composition and increase T-cell migration to the lungs

Methods for preventing a Coronavirus and/or Influenza infection in a subject may comprise administering to the subject a therapeutically effective amount of a hybrid vaccine composition according to the present invention. In some embodiments, the composition elicits an immune response in the subject. In some embodiments, the composition induces memory B and T cells. In some embodiments, the composition induces resident memory T cells (Trm). In some embodiments, the composition prevents virus replication, e.g., in the areas where the virus normally replicates, such as the lungs, brain, heart, and kidney. In some embodiments, the composition prevents a cytokine storm, e.g., in the areas where the virus normally replicates, such as the lungs, brain, heart, and kidney. In some embodiments, the composition prevents inflammation or an inflammatory response, e.g., in the areas where the virus normally replicates, such as the lungs, brain, heart, and kidney. In some embodiments, the composition improves the homing and retention of T cells, e.g., in the areas where the virus normally replicates, such as the lungs, brain, heart, and kidney.

Methods for preventing a Coronavirus and/or Influenza infection prophylactically in a subject may comprise administering to the subject a prophylactically effective amount of a hybrid vaccine composition according to the present invention. In some embodiments, the composition elicits an immune response in the subject. In some embodiments, the composition induces memory B and T cells. In some embodiments, the composition induces resident memory T cells (Trm). In some embodiments, the composition prevents virus replication, e.g., in the areas where the virus normally replicates, such as the lungs, brain, heart, and kidney. In some embodiments, the composition prevents a cytokine storm, e.g., in the areas where the virus normally replicates, such as the lungs, brain, heart, and kidney. In some embodiments, the composition prevents inflammation or an inflammatory response, e.g., in the areas where the virus normally replicates, such as the lungs, brain, heart, and kidney. In some embodiments, the composition improves the homing and retention of T cells, e.g., in the areas where the virus normally replicates, such as the lungs, brain, heart, and kidney.

Methods for eliciting an immune response in a subject may comprise administering to the subject a hybrid vaccine composition according to the present invention, wherein the composition elicits an immune response in the subject. In some embodiments, the composition induces memory B and T cells. In some embodiments, the composition induces resident memory T cells (Trm). In some embodiments, the composition prevents virus replication, e.g., in the areas where the virus normally replicates, such as the lungs, brain, heart, and kidney. In some embodiments, the composition prevents a cytokine storm, e.g., in the areas where the virus normally replicates, such as the lungs, brain, heart, and kidney. In some embodiments, the composition prevents inflammation or an inflammatory response, e.g., in the areas where the virus normally replicates, such as the lungs, brain, heart, and kidney. In some embodiments, the composition improves the homing and retention of T cells, e.g., in the areas where the virus normally replicates, such as the lungs, brain, heart, and kidney.

Methods for prolonging an immune response induced by a hybrid vaccine composition of the present invention and increasing T cell migration to particular tissues (e.g., lung, brain, heart, kidney, etc.) may comprise co-expressing a T-cell attracting chemokine, a composition that promotes T cell proliferation, and a vaccine composition (e.g., antigen) according to the present invention.

Methods for prolonging the retention of memory T cells into the lungs induced by a vaccine composition of the present invention and increasing virus-specific tissue-resident memory T-cells (TRM cells) may comprise co-expressing a T-cell attracting chemokine, a composition that promotes T cell proliferation, and a vaccine composition (e.g., antigen) according to the present invention.

The vaccine composition may be administered through standard means, e.g., through an intravenous route (i.v.), an intranasal route (i.n.), or a sublingual route (s.l.) route.

In certain embodiments, the method comprises administering to the subject a second (e.g., booster) dose. The second dose may comprise the same vaccine composition or a different vaccine composition. Additional doses of one or more vaccine compositions may be administered.

### Sequential Vaccine Delivery Methodology

In some embodiments, the present invention features a method of delivering the vaccine to induce heterologous immunity in a subject (e.g., prime/boost). In some embodiments, the method comprises administering a first hybrid vaccine composition dose using a first delivery system. In further embodiments, the method comprises administering a second vaccine composition dose using a second delivery system. In some embodiments, the second composition is administered 8 days after administration of the first composition. In some embodiments, the second composition is administered 9 days after administration of the first composition. In some embodiments, the second composition is administered 10 days after administration of the first composition. In some embodiments, the second composition is administered 11 days after administration of the first composition. In some embodiments, the second composition is administered 12 days after administration of the first composition. In some embodiments, the second composition is administered 13 days after administration of the first composition. In some embodiments, the second composition is administered 14 days after administration of the first composition. In some embodiments, the second composition is administered from 14 to 30 days after administration of the first composition. In some embodiments, the second composition is administered from 30 to 60 days after administration of the first composition. In other embodiments, the first delivery system and the second delivery system are different. In some embodiments, the peptide vaccine composition is administered 14 days after the administration of the first vaccine composition dose. In some embodiments, the peptide vaccine composition is administered 30 or 60 days after the administration of the first vaccine composition dose.

In some embodiments, the first delivery system or the second delivery system comprises an mRNA, a modified mRNA, or a peptide vector. In other embodiments, the peptide vector comprises an adenovirus or an adeno-associated virus vector.

In some embodiments, the present invention features a method of delivering the vaccine to induce heterologous immunity in a subject (i.e., prime/pull). In some embodiments, the method comprises administering a hybrid vaccine composition. In further embodiments, the method comprises administering at least one T-cell attracting chemokine after administering the hybrid vaccine composition. In some embodiments, the T-cell attracting chemokine is administered 8 days after the vaccine composition is administered. In some embodiments, the T-cell attracting chemokine is administered 9 days after the vaccine composition is administered. In some embodiments, the T-cell attracting chemokine is administered 10 days after the vaccine composition is administered. In some embodiments, the T-cell attracting chemokine is administered 11 days after the vaccine composition is administered. In some embodiments, the T-cell attracting chemokine is administered 12 days after the vaccine composition is administered. In some embodiments, the T-cell attracting chemokine is administered 13 days after the vaccine composition is administered. In some embodiments, the T-cell attracting chemokine is administered 14 days after the vaccine composition is administered. In some embodiments, the T-cell attracting chemokine is administered from 14 to 30 days after administration of the vaccine composition. In some embodiments, the T-cell attracting chemokine is administered from 30 to 60 days after administration of the vaccine composition. In some embodiments, the T cell-attracting chemokine composition is administered 8 to 14 days after the administration of the final vaccine composition dose. In some embodiments, the cell-attracting chemokine composition is administered 30 or 60 days after the administration of the final vaccine composition dose.

The present invention also features a novel “prime, pull, and boost” strategy. In other embodiments, the present invention features a method to increase the size and maintenance of lung-resident B-cells, CD4+ T cells, and CD8+ T cells to protect against SARS-CoV-2. In some embodiments, the method comprises administering a hybrid vaccine composition. In other embodiments, the method comprises administering at least one T-cell attracting chemokine after administering the hybrid vaccine composition. In further embodiments, the method comprises administering at least one cytokine after administering the T-cell attracting chemokine. In some embodiments, the T-cell attracting chemokine is administered 14 days after administering the hybrid vaccine composition. In other embodiments, the cytokine is administered 10 days after administering the T-cell attracting chemokine. In some embodiments, the T-cell attracting chemokine is administered 8 days after the vaccine composition is administered. In some embodiments, the T-cell attracting chemokine is administered 9 days after the vaccine composition is administered. In some embodiments, the T-cell attracting chemokine is administered 10 days after the vaccine composition is administered. In some embodiments, the T-cell attracting chemokine is administered 11 days after the vaccine composition is administered. In some embodiments, the T-cell attracting chemokine is administered 12 days after the vaccine composition is administered. In some embodiments, the T-cell attracting chemokine is administered 13 days after the vaccine composition is administered. In some embodiments, the T-cell attracting chemokine is administered 14 days after the vaccine composition is administered. In some embodiments, the T-cell attracting chemokine is administered from 14 to 30 days after administration of the vaccine composition. In some embodiments, the T-cell attracting chemokine is administered from 30 to 60 days after administration of the vaccine composition. In some embodiments, the cytokine is administered 8 days after administering the T-cell attracting chemokine. In some embodiments, the cytokine is administered 9 days after administering the T-cell attracting chemokine. In some embodiments, the cytokine is administered 10 days after administering the T-cell attracting chemokine. In some embodiments, the cytokine is administered 11 days after administering the T-cell attracting chemokine. In some embodiments, the cytokine is administered 12 days after administering the T-cell attracting chemokine. In some embodiments, the cytokine is administered 13 days after administering the T-cell attracting chemokine. In some embodiments, the cytokine is administered 14 days after administering the T-cell attracting chemokine. In some embodiments, the cytokine is administered from 14 to 30 days after administering the T-cell attracting chemokine. In some embodiments, the cytokine is administered from 30 to 60 days after administering the T-cell attracting chemokine. In some embodiments, the cytokine composition is administered 8 to 14 days after the administration of the T cell-attracting chemokine. In some embodiments, the cytokine composition is administered 30 or 60 days after the administration of the T cell-attracting chemokine.

The present invention further features a novel “prime, pull, and keep” strategy. In further embodiments, the present invention features a method to increase the size and maintenance of lung-resident B-cells, CD4+ T cells, and CD8+ T cells to protect against SARS-CoV-2. In some embodiments, the method comprises administering a hybrid vaccine composition. In other embodiments, the method comprises administering at least one T-cell attracting chemokine after administering the hybrid vaccine composition. In further embodiments, the method comprises administering at least one mucosal chemokine after administering the T-cell attracting chemokine. In some embodiments, the T-cell attracting chemokine is administered 14 days after administering the hybrid vaccine composition. In other embodiments, the mucosal chemokines are administered 10 days after administering the T-cell attracting chemokine. In some embodiments, the T-cell attracting chemokine is administered 8 days after the vaccine composition is administered. In some embodiments, the T-cell attracting chemokine is administered 9 days after the vaccine composition is administered. In some embodiments, the T-cell attracting chemokine is administered 10 days after the vaccine composition is administered. In some embodiments, the T-cell attracting chemokine is administered 11 days after the vaccine composition is administered. In some embodiments, the T-cell attracting chemokine is administered 12 days after the vaccine composition is administered. In some embodiments, the T-cell attracting chemokine is administered 13 days after the vaccine composition is administered. In some embodiments, the T-cell attracting chemokine is administered 14 days after the vaccine composition is administered. In some embodiments, the T-cell attracting chemokine is administered from 14 to 30 days after administration of the vaccine composition. In some embodiments, the T-cell attracting chemokine is administered from 30 to 60 days after administration of the vaccine composition. In some embodiments, the mucosal chemokine is administered 8 days after administering the T-cell attracting chemokine. In some embodiments, the mucosal chemokine is administered 9 days after administering the T-cell attracting chemokine. In some embodiments, the mucosal chemokine is administered 10 days after administering the T-cell attracting chemokine. In some embodiments, the mucosal chemokine is administered 11 days after administering the T-cell attracting chemokine. In some embodiments, the mucosal chemokine is administered 12 days after administering the T-cell attracting chemokine. In some embodiments, the mucosal chemokine is administered 13 days after administering the T-cell attracting chemokine. In some embodiments, the mucosal chemokine is administered 14 days after administering the T-cell attracting chemokine. In some embodiments, the mucosal chemokine is administered from 14 to 30 days after administering the T-cell attracting chemokine. In some embodiments, the mucosal chemokine is administered from 30 to 60 days after administering the T-cell attracting chemokine. In some embodiments, the mucosal chemokine composition is administered 8 to 14 days after the administration of the T cell-attracting chemokine. In some embodiments, the mucosal cytokine composition is administered 30 or 60 days after the administration of the T cell-attracting chemokine.

In some embodiments, the mucosal chemokines may comprise CCL25, CCL28, CXCL14, CXCL17, or a combination thereof. In some embodiments, the T-cell attracting chemokines may comprise CCL5, CXCL9, CXCL10, CXCL11, or a combination thereof. In some embodiments, the cytokines may comprise IL-15, IL-2, IL-7, or a combination thereof.

In some embodiments, the efficacy (or effectiveness) of a vaccine composition herein is greater than 60%. In some embodiments, the efficacy (or effectiveness) of a vaccine composition herein is greater than 70%. In some embodiments, the efficacy (or effectiveness) of a vaccine composition herein is greater than 80%. In some embodiments, the efficacy (or effectiveness) of a vaccine composition herein is greater than 90%. In some embodiments, the efficacy (or effectiveness) of a vaccine composition herein is greater than 95%.

Vaccine efficacy may be assessed using standard analyses (see, e.g., Weinberg et al., J Infect Dis. 2010 Jun. 1; 201 (11): 1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with the use of the following formulas: Efficacy=(ARU-ARV)/ARU×100; and Efficacy=(1−RR)×100.

Likewise, vaccine effectiveness may be assessed using standard analyses (see, e.g., Weinberg et al., J Infect Dis. 2010 Jun. 1; 201 (11): 1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with the use of the odds ratio (OR) for developing infection despite vaccination: Effectiveness=(1−OR)×100.

In some embodiments, the vaccine immunizes the subject against Coronavirus and/or Influenza for up to 1 year. In some embodiments, the vaccine immunizes the subject against Coronavirus and/or Influenza for up to 2 years. In some embodiments, the vaccine immunizes the subject against Coronavirus and/or Influenza for more than 1 year, more than 2 years, more than 3 years, more than 4 years, or for 5-10 years.

In some embodiments, the subject is a young adult between the ages of about 20 years and about 50 years (e.g., about 20, 25, 30, 35, 40, 45, or 50 years old).

In some embodiments, the subject is an elderly subject about 60 years old, about 70 years old, or older (e.g., about 60, 65, 70, 75, 80, 85, or 90 years old).

In some embodiments, the subject is about 5 years old or younger. For example, the subject may be between the ages of about 1 year and about 5 years (e.g., about 1, 2, 3, 5, or 5 years) or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject was born full term (e.g., about 37-42 weeks). In some embodiments, the subject was born prematurely, for example, at about 36 weeks of gestation or earlier (e.g., about 36, 35, 34, 33, 32, 31, 30, 29, 28, 27, 26, or 25 weeks). For example, the subject may have been born at about 32 weeks of gestation or earlier. In some embodiments, the subject was born prematurely between about 32 weeks and about 36 weeks of gestation. In such subjects, a vaccine may be administered later in life, for example, at the age of about 6 months to about 5 years or older.

In some embodiments, the subject is pregnant (e.g., in the first, second, or third trimester) when administered a vaccine.

In some embodiments, the subject has been exposed to Coronavirus or Influenza. In some embodiments, the subject is infected with Coronavirus or Influenza. In some embodiments, the subject is at risk of infection by a Coronavirus or Influenza.

In some embodiments, the subject is immunocompromised (has an impaired immune system, e.g., has an immune disorder or autoimmune disorder).

### Pharmaceutical Carriers

In certain embodiments, the vaccine composition further comprises a pharmaceutical carrier. Pharmaceutical carriers are well known to one of ordinary skill in the art. For example, in certain embodiments, the pharmaceutical carrier is selected from the group consisting of water, an alcohol, a natural or hardened oil, a natural or hardened wax, a calcium carbonate, a sodium carbonate, a calcium phosphate, kaolin, talc, lactose and combinations thereof. In some embodiments, the pharmaceutical carrier may comprise a lipid nanoparticle, an adenovirus vector, or an adeno-associated virus vector. In some embodiments, the vaccine composition is constructed using an adeno-associated virus vectors-based antigen delivery system.

Also provided herein is a vaccine of any one of the foregoing paragraphs, formulated in a nanoparticle (e.g., a lipid nanoparticle). In some embodiments, the nanoparticle has a mean diameter of 50-200 nm. In some embodiments, the nanoparticle is a lipid nanoparticle. In some embodiments, the lipid nanoparticle comprises a cationic lipid, a PEG-modified lipid, a sterol, and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid, the non-cationic lipid is a neutral lipid, and the sterol is a cholesterol. In some embodiments, the cationic lipid is selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319).

For example, the present invention may feature a pharmaceutical composition. In some embodiments, the pharmaceutical composition may comprise a plurality of lipid nanoparticles; where a first lipid nanoparticle comprises three messenger ribonucleic acids (mRNAs) encapsulated therein, and each mRNA comprises an open reading frame encoding a Coronavirus protein selected from a group comprising, consisting essentially, or consisting of: an NSP2 protein, an NSP14 protein, and a Nucleoprotein, a second lipid nanoparticle comprising an mRNA comprising an open reading frame encoding a Coronavirus Spike protein or portion thereof encapsulated therein and a third lipid nanoparticle comprising an mRNA comprising an open reading frame encoding an Influenza protein or portion thereof encapsulated therein. Likewise, the pharmaceutical composition may comprise a plurality of lipid nanoparticles; where a first lipid nanoparticle comprises three messenger ribonucleic acids (mRNAs) encapsulated therein, and each mRNA comprises an open reading frame encoding an entire Coronavirus protein selected from a group comprising, consisting essentially, or consisting of: an NSP2 protein, an NSP14 protein, and a Nucleoprotein, second lipid nanoparticle comprising an mRNA comprising an open reading frame encoding a Coronavirus Spike protein or portion thereof encapsulated therein, and a third lipid nanoparticle comprising an mRNA comprising an open reading frame encoding an Influenza protein or portion thereof encapsulated therein.

Alternatively, the pharmaceutical composition may comprise a plurality of lipid nanoparticles; where a first lipid nanoparticle comprises three messenger ribonucleic acids (mRNAs) encapsulated therein, and each mRNA comprises an open reading frame encoding a Coronavirus protein selected from a group comprising, consisting essentially, or consisting of: an NSP2 protein, an NSP14 protein, a Nucleoprotein and a Spike protein, a second lipid nanoparticle comprising an mRNA comprising an open reading frame encoding an Influenza protein or portion thereof encapsulated therein. Likewise, the pharmaceutical composition may comprise a plurality of lipid nanoparticles; where a first lipid nanoparticle comprises three messenger ribonucleic acids (mRNAs) encapsulated therein, and each mRNA comprises an open reading frame encoding an entire Coronavirus protein selected from a group comprising, consisting essentially, or consisting of: an NSP2 protein, an NSP14 protein, a Nucleoprotein and a Spike protein and a second lipid nanoparticle comprising an mRNA comprising an open reading frame encoding an Influenza protein or portion thereof encapsulated therein.

In some embodiments, the first lipid nanoparticle comprises two mRNAs (preferably different) encapsulated therein. In some embodiments, the first lipid nanoparticle comprises two or more mRNAs (preferably different) encapsulated therein. In some embodiments, the first lipid nanoparticle comprises three mRNAs (preferably different) encapsulated therein. In some embodiments, the first lipid nanoparticle comprises three or more mRNAs (preferably different) encapsulated therein. In some embodiments, the first lipid nanoparticle comprises four mRNAs (preferably different) encapsulated therein. In some embodiments, the first lipid nanoparticle comprises four or more mRNAs (preferably different) encapsulated therein. In some embodiments, the first lipid nanoparticle comprises five mRNAs (preferably different) encapsulated therein. In some embodiments, the first lipid nanoparticle comprises five or more mRNAs (preferably different) encapsulated therein.

In some embodiments, the second lipid nanoparticle comprises one mRNA encapsulated therein. In some embodiments, the second lipid nanoparticle comprises two or more mRNAs (preferably different) encapsulated therein. In some embodiments, the second lipid nanoparticle comprises three mRNAs (preferably different) encapsulated therein. In some embodiments, the second lipid nanoparticle comprises three or more mRNAs (preferably different) encapsulated therein. In some embodiments, the second lipid nanoparticle comprises four mRNAs (preferably different) encapsulated therein. In some embodiments, the second lipid nanoparticle comprises four or more mRNAs (preferably different) encapsulated therein.

In some embodiments, the third lipid nanoparticle comprises one mRNA encapsulated therein. In some embodiments, the third lipid nanoparticle comprises two or more mRNAs (preferably different) encapsulated therein. In some embodiments, the third lipid nanoparticle comprises three mRNAs (preferably different) encapsulated therein. In some embodiments, the third lipid nanoparticle comprises three or more mRNAs (preferably different) encapsulated therein. In some embodiments, the third lipid nanoparticle comprises four mRNAs (preferably different) encapsulated therein. In some embodiments, the third lipid nanoparticle comprises four or more mRNAs (preferably different) encapsulated therein.

The pharmaceutical composition described herein may comprise a single lipid nanoparticle (e.g., a first nanoparticle).

Additional sequences and details about methods to select sequences can be found in U.S. Application No. PCT/US21/27355, U.S. Application No. PCT/US21/27340, U.S. Application No. PCT/US21/27341 the specifications of which are hereby incorporated in their entirety by reference.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A hybrid Coronavirus-influenza vaccine composition, the composition comprising, or comprising a sequence encoding, at least a conserved portion of a Coronavirus spike(S) protein and at least a conserved portion of at least one Influenza hemagglutinin (HA) protein.

2. The composition of claim 1, wherein the conserved portion of the Coronavirus spike(S) protein is highly conserved among human and animal Coronaviruses.

3. The composition of claim 2, wherein the conserved portion of the Coronavirus spike(S) protein is derived from one or more of: one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animals receptive to coronaviruses; or one or more coronaviruses that cause the common cold; or one or more coronaviruses that cause past, current, and future outbreaks.

4. The composition of claim 3, wherein the one or more SARS-CoV-2 human variants in current circulation are selected from: variant B.1.177; variant B.1.160, variant B.1.1.7; variant B.1.351; variant P.1; variant B.1.427/B.1.429; variant B.1.258; variant B.1.221; variant B.1.367; variant B.1.1.277; variant B.1.1.302; variant B.1.525; variant B.1.526, variant S: 677H; variant S: 677P; B.1.617.2-Delta, variant B.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5) and the one or more coronaviruses that cause the common cold are selected from: 229E alpha coronavirus, NL63 alpha coronavirus, OC43 beta coronavirus, and HKU1 beta coronavirus.

5. The composition of claim 1, wherein the conserved portion of the Coronavirus spike(S) protein is derived from a full-length spike glycoprotein.

6. The composition of claim 1, wherein the conserved portion of the Coronavirus spike(S) protein is derived from a partial spike glycoprotein.

7. The composition of claim 1, wherein the conserved portion of the Coronavirus spike(S) protein is encoded by one of SEQ ID NOs: 1, 3, 5-7.

8. The composition of claim 1, wherein the conserved portion of the influenza hemagglutinin (HA) protein is highly conserved among human influenza viruses.

9. The composition of claim 8, wherein the portion of the influenza hemagglutinin (HA) protein is derived from one or more of: H1N1 virus strain, H3N2 virus strain, or influenza B virus strains, wherein the H1N1 virus strains or variants are selected from: OK384178.1, OM642156.1, OM654386.1, OL840606.1, OK625377.1, OM865246.1, OM935941.1, OM642158.1, OM935953.1, MW840068.1, MW839847.1, MW839825.1, MW930730.1, MT227010.1, LC638096.1, LC638077.1, LC637999.1, and LC645067.1; wherein the H3N2 virus strains or variants are selected from: MZ005227.1, MW849238.1, MZ203409.1, MZ198318.1, MZ198312.1, MZ198295.1, MZ198289.1, MZ198265.1, MW789449.1, MW798370.1, MW790182.1, MW789645.1, MW789778.1, MW789685.1, MW789659.1, and MW790001.1 and wherein the influenza B virus strains or variants are selected from: M10298.1, MT738525.1, MT808088.1, MT056751.1, MT314641.1, MT874090.1, MT242979.1, MT315665.1, MT1055640.1, MT057563.1, MT056955.1, MT243019.1, MT306916.1, MT057571.1, MT314793.1, MT343026.1, MT874109.1, MT243795.1, MT315769.1, and KX885875.1.

10. The composition of claim 1, wherein the conserved portion of the influenza hemagglutinin (HA) protein is encoded by one of SEQ ID NOs: 12, 14-16.

11. The composition of claim 1, wherein the composition is encoded by one of SEQ ID NO: 21-53.

12. The composition of claim 1 further comprising or encoding a T cell attracting chemokine, wherein the T cell attracting chemokine is CCL5, CXCL9, CXCL10, CXCL11, or a combination thereof.

13. The composition of claim 1 further comprising or encoding a composition that promotes T cell proliferation and T-cell memory, wherein the composition that promotes T cell proliferation and memory is IL-7, IL-2, or IL-15.

14. A hybrid Coronavirus-Influenza vaccine composition, the composition comprising, or comprising a sequence encoding:
a. at least a conserved portion of at least one Coronavirus protein, the Coronavirus protein being selected from: a Spike protein, a Nucleocapsid protein, NSP2, NSP3, NSP14, or combination thereof; and
b. at least a conserved portion of at least one influenza hemagglutinin (HA) protein.

15. An Influenza vaccine composition, the composition comprising, or comprising a sequence encoding: at least a conserved portion of at least one influenza hemagglutinin (HA) protein.

16. The composition of claim 15, wherein the portion of the influenza hemagglutinin (HA) protein is highly conserved among human influenza viruses.

17. The composition of claim 16, wherein the portion of the influenza hemagglutinin (HA) protein is derived from one or more of: H1N1 virus strain, H3N2 virus strain, or influenza B virus strains, wherein the H1N1 virus strains or variants are selected from: OK384178.1, OM642156.1, OM654386.1, OL840606.1, OK625377.1, OM865246.1, OM935941.1, OM642158.1, OM935953.1, MW840068.1, MW839847.1, MW839825.1, MW930730.1, MT227010.1, LC638096.1, LC638077.1, LC637999.1, and LC645067.1; wherein the H3N2 virus strains or variants are selected from: MZ005227.1, MW849238.1, MZ203409.1, MZ198318.1, MZ198312.1, MZ198295.1, MZ198289.1, MZ198265.1, MW789449.1, MW798370.1, MW790182.1, MW789645.1, MW789778.1, MW789685.1, MW789659.1, and MW790001.1 and wherein the influenza B virus strains or variants are selected from: M10298.1, MT738525.1, MT808088.1, MT056751.1, MT314641.1, MT874090.1, MT242979.1, MT315665.1, MT1055640.1, MT057563.1, MT056955.1, MT243019.1, MT306916.1, MT057571.1, MT314793.1, MT343026.1, MT874109.1, MT243795.1, MT315769.1, and KX885875.1.

18. The composition of claim 15, wherein the portion of the influenza hemagglutinin (HA) protein is encoded by one of SEQ ID NOs: 12, 14-16.


SEQUENCE LISTING (69 sequences)

SEQ ID NO: 1 - DNA, 3771 bp, synthetic construct
  source 1..3771: mol_type = other DNA
aacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacc

SEQ ID NO: 2 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVTRAGCLIGAEHVNNSYECDIPIGAGICASYQTQTNRDPQTLEILDITPCSFGGVSVITPGTNTSNQVAVLYQDVNCTEVPVAIHADQLTPTWRVYSTGSNVFQSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 3771 bp, synthetic construct
  source 1..3771: mol_type = other DNA
aacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacc

SEQ ID NO: 4 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - DNA, 3762 bp, synthetic construct
  source 1..3762: mol_type = other DNA
aacctgatcaccaggacccagagctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctgggcagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcaggggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacggcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagtacgtgaacaacagctacgagtgcgacatccccatcggcgccggcatctgcgccagctaccagacccagaccaagagccaccagcaggcccagagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccacaacgcccaggccctgaacaccctggtgaagcagctgagcagcaagttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacc

SEQ ID NO: 6 - DNA, 3762 bp, synthetic construct
  source 1..3762: mol_type = other DNA
aacctgatcaccaggacccagagctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctgggcagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcaggggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacggcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagtacgtgaacaacagctacgagtgcgacatccccatcggcgccggcatctgcgccagctaccagacccagaccaagagccaccagcaggcccagagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacc

SEQ ID NO: 7 - DNA, 2099 bp, synthetic construct
  source 1..2099: mol_type = other DNA
taccaagctactagagtagtggtactttcatttgagcttctaaatgcacctgccacagtgtgtggaccaaaattgtccacatcactaattaagaaccagtgtgtcaattttaatttcaatggactcaagggtactggtgtgttgactgactcgtccaaaaagtttcagtcttttcaacaatttggaagggatgcatctgattttactgactcagtacgcgaccctcagacacttcaaatacttgacatttcaccatgttcatttggtggtgtgagtgtaataacaccaggaacaaatgcttcatctgaagtagccgttctataccaagatgtaaactgcactgatgttcccacggccatacgtgctgaccaactcacacctgcttggcgtgtttactctgctggagtaaatgtgtttcaaactcaggctggctgtttaataggagcggaacatgtcaatgcttcatatgagtgtgacattcccattggtgcaggcatttgtgctagttaccatacagcttcccttttacgtaatacaggccagaaatcaattgtggcctatactatgtcacttggtgctgaaaactcaattgcttatgctaataactcaattgccatacctacaaatttttcaatcagtgtcacaactgaagtgatgcctgtttcaatggctaagacatcagtagattgtacaatgtacatctgtggtgactctcaggagtgcagcaacttactacttcagtatggtagcttttgcacacaattaaatcgtgccctttcaggcattgctgttgaacaggacaaaaacactcaagaggtttttgcccaagttaaacaaatgtataagacaccagccataaaagattttggtggctttaatttctcacaaatattgcctgacccttctaagccaacaaaaagatcatttattgaggatttactcttcaacaaagtgactctcgctgatgctggctttatgaagcaatacggcgaatgcctaggcgatattagtgctagagatctcatttgtgcgcagaagttcaatggactcactgtccttccacctctactcacggatgaaatgattgctgcttacaccgccgctcttgtcagcggtactgctactgctggttggacatttggtgcaggtgctgctctacaaataccttttgctatgcaaatggcttataggttcaatggcattggagttactcaaaatgttctctatgagaaccagaagcagatcgctaaccaatttaacaaggcgatcagtcaaattcaagaatcacttactactacttcaactgcattgggcaagctgcaagacgtcgtcaaccagaatgctcaagcattgaacacacttgttaaacaactaagttctaactttggtgcaatttcaagtgttttaaatgacattctgtctcgactygacaaagttgaggctgaagtgcaaattgataggttgattactggcagattacaaagccttcagacctatgtaacacaacaactaatcagagctgctgaaatcagagcttctgccaatcttgctgccactaagatgtccgagtgtgttcttggacaatcaaaaagagttgacttttgtggaaaaggctatcatcttatgtctttccctcaagcagccccacatggtgtcgtcttcttacatgtcacatacgtgccatcgcaagaaagaaacttcaccactgccccagcaatctgccatcaaggcaaggcacacttccctcgtgaaggtgtttttgtatctaatggcacttcttggtttatcacacagaggaacttcttttcaccacaaataattacaacagacaatacatttgtctctggaaattgtgatgtcgttattggcatcatcaacaatactgtttatgatcctctgcaacctgagcttgactcatttaaagaagagctggacaagtacttcaaaaaccacacgtcacctgatgtrgatcttggcgacatctcaggcattaatgcttcagtcgtcaatattcaaaaagaaattgaccgcctcaatgaggttgccaaaaatctaaatgaatcgctcatcgatcttcaagaacttggaaaatatgagca

SEQ ID NO: 8 - DNA, 1914 bp, synthetic construct
  source 1..1914: mol_type = other DNA
gcctacaccaggtacgtggacaacaacttctgcggccccgacggctaccccctggagtgcatcaaggacctgctggccagggccggcaaggccagctgcaccctgagcgagcagctggacttcatcgacaccaagaggggcgtgtactgctgcagggagcacgagcacgagatcgcctggtacaccgagaggagcgagaagagctacgagctgcagacccccttcgagatcaagctggccaagaagttcgacaccttcaacggcgagtgccccaacttcgtgttccccctgaacagcatcatcaagaccatccagcccagggtggagaagaagaagctggacggcttcatgggcaggatcaggagcgtgtaccccgtggccagccccaacgagtgcaaccagatgtgcctgagcaccctgatgaagtgcgaccactgcggcgagaccagctggcagaccggcgacttcgtgaaggccacctgcgagttctgcggcaccgagaacctgaccaaggagggcgccaccacctgcggctacctgccccagaacgccgtggtgaagatctactgccccgcctgccacaacagcgaggtgggccccgagcacagcctggccgagtaccacaacgagagcggcctgaagaccatcctgaggaagggcggcaggaccatcgccttcggcggctgcgtgttcagctacgtgggctgccacaacaagtgcgcctactgggtgcccagggccagcgccaacatcggctgcaaccacaccggcgtggtgggcgagggcagcgagggcctgaacgacaacctgctggagatcctgcagaaggagaaggtgaacatcaacatcgtgggcgacttcaagctgaacgaggagatcgccatcatcctggccagcttcagcgccagcaccagcgccttcgtggagaccgtgaagggcctggactacaaggccttcaagcagatcgtggagagctgcggcaacttcaaggtgaccaagggcaaggccaagaagggcgcctggaacatcggcgagcagaagagcatcctgagccccctgtacgccttcgccagcgaggccgccagggtggtgaggagcatcttcagcaggaccctggagaccgcccagaacagcgtgagggtgctgcagaaggccgccatcaccatcctggacggcatcagccagtacagcctgaggctgatcgacgccatgatgttcaccagcgacctggccaccaacaacctggtggtgatggcctacatcaccggcggcgtggtgcagctgaccagccagtggctgaccaacatcttcggcaccgtgtacgagaagctgaagcccgtgctggactggctggaggagaagttcaaggagggcgtggagttcctgagggacggctgggagatcgtgaagttcatcagcacctgcgcctgcgagatcgtgggcggccagatcgtgacctgcgccaaggagatcaaggagagcgtgcagaccttcttcaagctggtgaacaagttcctggccctgtgcgccgacagcatcatcatcggcggcgccaagctgaaggccctgaacctgggcgagaccttcgtgacccacagcaagggcctgtacaggaagtgcgtgaagagcagggaggagaccggcctgctgatgcccctgaaggcccccaaggagatcatcttcctggagggcgagaccctgcccaccgaggtgctgaccgaggaggtggtgctgaagaccggcgacctgcagcccctggagcagcccaccagcgaggccgtggaggcccccctggtgggcacccccgtgtgcatcaacggcctgatgctgctggagatcaaggacaccgagaagtactgcgccctggcccccaacatgatggtgaccaacaacaccttcaccctgaagggcggc

SEQ ID NO: 9 - DNA, 2286 bp, synthetic construct
  source 1..2286: mol_type = other DNA
gcccccaccaaggtgaccttcggcgacgacaccgtgatcgaggtgcagggctacaagagcgtgaacatcaccttcgagctggacgagaggatcgacaaggtgctgaacgagaagtgcagcgcctacaccgtggagctgggcaccgaggtgaacgagttcgcctgcgtggtggccgacgccgtgatcaagaccctgcagcccgtgagcgagctgctgacccccctgggcatcgacctggacgagtggagcatggccacctactacctgttcgacgagagcggcgagttcaagctggccagccacatgtactgcagcttctacccccccgacgaggacgaggaggagggcgactgcgaggaggaggagttcgagcccagcacccagtacgagtacggcaccgaggacgactaccagggcaagcccctggagttcggcgccaccagcgccgccctgcagcccgaggaggagcaggaggaggactggctggacgacgacagccagcagaccgtgggccagcaggacggcagcgaggacaaccagaccaccaccatccagaccatcgtggaggtgcagccccagctggagatggagctgacccccgtggtgcagaccatcgaggtgaacagcttcagcggctacctgaagctgaccgacaacgtgtacatcaagaacgccgacatcgtggaggaggccaagaaggtgaagcccaccgtggtggtgaacgccgccaacgtgtacctgaagcacggcggcggcgtggccggcgccctgaacaaggccaccaacaacgccatgcaggtggagagcgacgactacatcgccaccaacggccccctgaaggtgggcggcagctgcgtgctgagcggccacaacctggccaagcactgcctgcacgtggtgggccccaacgtgaacaagggcgaggacatccagctgctgaagagcgcctacgagaacttcaaccagcacgaggtgctgctggcccccctgctgagcgccggcatcttcggcgccgaccccatccacagcctgagggtgtgcgtggacaccgtgaggaccaacgtgtacctggccgtgttcgacaagaacctgtacgacaagctggtgagcagcttcctggagatgaagagcgagaagcaggtggagcagaagatcgccgagatccccaaggaggaggtgaagcccttcatcaccgagagcaagcccagcgtggagcagaggaagcaggacgacaagaagatcaaggcctgcgtggaggaggtgaccaccaccctggaggagaccaagttcctgaccgagaacctgctgctgtacatcgacatcaacggcaacctgcaccccgacagcgccaccctggtgagcgacatcgacatcaccttcctgaagaaggacgccccctacatcgtgggcgacgtggtgcaggagggcgtgctgaccgccgtggtgatccccaccaagaaggccggcggcaccaccgagatgctggccaaggccctgaggaaggtgcccaccgacaactacatcaccacctaccccggccagggcctgaacggctacaccgtggaggaggccaagaccgtgctgaagaagtgcaagagcgccttctacatcctgcccagcatcatcagcaacgagaagcaggagatcctgggcaccgtgagctggaacctgagggagatgctggcccacgccgaggagaccaggaagctgatgcccgtgtgcgtggagaccaaggccatcgtgagcaccatccagaggaagtacaagggcatcaagatccaggagggcgtggtggactacggcgccaggttctacttctacaccagcaagaccaccgtggccagcctgatcaacaccctgaacgacctgaacgagaccctggtgaccatgcccctgggctacgtgacccacggcctgaacctggaggaggccgccaggtacatgaggagcctgaaggtgcccgccaccgtgagcgtgagcagccccgacgccgtgaccgcctacaacggctacctgaccagcagcagcaagacccccgaggagcacttcatcgagaccatcagcctggccggcagctacaaggactggagctacagcggccagagcacccagctgggcatcgagttcctgaagaggggcgacaagagcgtgtactacaccagcaaccccaccaccttccacctggacggcgaggtgatcaccttcgacaacctgaagaccctgctgagcctgagggaggtgaggaccatcaaggtgttcaccaccgtggacaacatcaacctgcac

SEQ ID NO: 10 - DNA, 1581 bp, synthetic construct
  source 1..1581: mol_type = other DNA
gccgagaacgtgaccggcctgttcaaggactgcagcaaggtgatcaccggcctgcaccccacccaggcccccacccacctgagcgtggacaccaagttcaagaccgagggcctgtgcgtggacatccccggcatccccaaggacatgacctacaggaggctgatcagcatgatgggcttcaagatgaactaccaggtgaacggctaccccaacatgttcatcaccagggaggaggccatcaggcacgtgagggcctggatcggcttcgacgtggagggctgccacgccaccagggaggccgtgggcaccaacctgcccctgcagctgggcttcagcaccggcgtgaacctggtggccgtgcccaccggctacgtggacacccccaacaacaccgacttcagcagggtgagcgccaagcccccccccggcgaccagttcaagcacctgatccccctgatgtacaagggcctgccctggaacgtggtgaggatcaagatcgtgcagatgctgagcgacaccctgaagaacctgagcgacagggtggtgttcgtgctgtgggcccacggcttcgagctgaccagcatgaagtacttcgtgaagatcggccccgagaggacctgctgcctgtgcgacaggagggccacctgcttcagcaccgccagcgacacctacgcctgctggcaccacagcatcggcttcgactacgtgtacaaccccttcatgatcgacgtgcagcagtggggcttcaccggcaacctgcagagcaaccacgacctgtactgccaggtgcacggcaacgcccacgtggccagctgcgacgccatcatgaccaggtgcctggccgtgcacgagtgcttcgtgaagagggtggactggaccatcgagtaccccatcatcggcgacgagctgaagatcaacgccgcctgcaggaaggtgcagcacatggtggtgaaggccgccctgctggccgacaagttccccgtgctgcacgacatcggcaaccccaaggccatcaagtgcgtgccccaggccgacgtggagtggaagttctacgacgcccagccctgcagcgacaaggcctacaagatcgaggagctgttctacagctacgccacccacagcgacaagttcaccgacggcgtgtgcctgttctggaactgcaacgtggacaggtaccccgccaacagcatcgtgtgcaggttcgacaccagggtgctgagcaacctgaacctgcccggctgcgacggcggcagcctgtacgtgaacaagcacgccttccacacccccgccttcgacaagagcgccttcgtgaacctgaagcagctgcccttcttctactacagcgacagcccctgcgagagccacggcaagcaggtggtgagcgacatcgactacgtgcccctgaagagcgccacctgcatcaccaggtgcaacctgggcggcgccgtgtgcaggcaccacgccaacgagtacaggctgtacctggacgcctacaacatgatgatcagcgccggcttcagcctgtgggtgtacaagcagttcgacacctacaacctgtggaacaccttcaccaggctgcag

SEQ ID NO: 11 - DNA, 1254 bp, synthetic construct
  source 1..1254: mol_type = other DNA
agcgacaacggcccccagaaccagaggaacgcccccaggatcaccttcggcggccccagcgacagcaccggcagcaaccagaacggcgagaggagcggcgccaggagcaagcagaggaggccccagggcctgcccaacaacaccgccagctggttcaccgccctgacccagcacggcaaggaggacctgaagttccccaggggccagggcgtgcccatcaacaccaacagcagccccgacgaccagatcggctactacaggagggccaccaggaggatcaggggcggcgacggcaagatgaaggacctgagccccaggtggtacttctactacctgggcaccggccccgaggccggcctgccctacggcgccaacaaggacggcatcatctgggtggccaccgagggcgccctgaacacccccaaggaccacatcggcaccaggaaccccgccaacaacgccgccatcgtgctgcagctgccccagggcaccaccctgcccaagggcttctacgccgagggcagcaggggcggcagccaggccagcagcaggagcagcagcaggagcaggaacagcagcaggaacagcacccccggcagcagcaggggcaccagccccgccaggatggccggcaacggcggcgacgccgccctggccctgctgctgctggacaggctgaaccagctggagagcaagatgagcggcaagggccagcagcagcagggccagaccgtgaccaagaagagcgccgccgaggccagcaagaagcccaggcagaagaggaccgccaccaaggcctacaacgtgacccaggccttcggcaggaggggccccgagcagacccagggcaacttcggcgaccaggagctgatcaggcagggcaccgactacaagcactggccccagatcgcccagttcgcccccagcgccagcgccttcttcggcatgagcaggatcggcatggaggtgacccccagcggcacctggctgacctacaccggcgccatcaagctggacgacaaggaccccaacttcaaggaccaggtgatcctgctgaacaagcacatcgacgcctacaagaccttcccccccaccgagcccaagaaggacaagaagaagaaggccgacgagacccaggccctgccccagaggcagaagaagcagcagaccgtgaccctgctgcccgccgccgacctggacgacttcagcaagcagctgcagcagagcatgagcagcgccgacagcacccaggcc

SEQ ID NO: 12 - DNA, 119 bp, synthetic construct
  source 1..119: mol_type = other DNA
ttcggagctattgctggtttcttggaaggaggatgggaaggaatgattgcaggttggcacggatacacatctcatggagcacatggagtagcagtggcagcagaccttaagagtaccca

SEQ ID NO: 13 - protein, 40 aa, synthetic construct
FGAIAGFLEGGWEGMIAGWHGYTSHGAHGVAVAADLKSTX

SEQ ID NO: 14 - DNA, 1701 bp, synthetic construct
  source 1..1701: mol_type = other DNA
atgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaa

SEQ ID NO: 15 - DNA, 1762 bp, synthetic construct
  source 1..1762: mol_type = other DNA
agcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctact

SEQ ID NO: 16 - DNA, 1847 bp, synthetic construct
  source 1..1847: mol_type = other DNA
attttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaat

SEQ ID NO: 17 - DNA, 589 bp, synthetic construct
  source 1..589: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 18 - DNA, 1733 bp, synthetic construct
  source 1..1733: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 19 - DNA, 635 bp, synthetic construct
  source 1..635: mol_type = other DNA
gtatagggctgtctgggagccactccagggccacagaaatcttgtctctgactcagggtattttgttttctgttttgtgtaaatgctcttctgactaatgcaaaccatgtgtccatagaaccagaagatttttccaggggaaaaggtaaggaggtggtgagagtgtcctgggtctgcccttccagggcttgccctgggttaagagccaggcaggaagctctcaagagcattgctcaagagtagagggggcctgggaggcccagggaggggatgggaggggaacacccaggctgcccccaaccagatgccctccaccctcctcaacctccctcccacggcctggagaggtgggaccaggtatggaggcttgagagcccctggttggaggaagccacaagtccaggaacatgggagtctgggcagggggcaaaggaggcaggaacaggccatcagccaggacaggtggtaaggcaggcaggagtgttcctgctgggaaaaggtgggatcaagcacctggagggctcttcagagcaaagacaaacactgaggtcgctgccactcctacagagcccccacgccccgcccagctataaggggccatgcaccaagcagggtacccaggctgcagaggtgcc

SEQ ID NO: 20 - DNA, 1559 bp, synthetic construct
  source 1..1559: mol_type = other DNA
catccatgcccatggcctcagatgccagccataagctgttgggttccaaacctcgactccaggctggactcacccctgtctcccccaccagcctgacacctccacctgggtatctaacgagcatctcaaactcaacctgcctgagacagaggaatcactatcccctcctcctccaaaaatatccttccatcacactccccatcttgtgctctgatttactaaacggccctgggccctctctttctcagggtctctgcttgcccagctatataataaaacaagtttgggacttcccaaccattcacccatggaaaaacagaagcaactcttcaaaggacagattcccaggatctgccctgggagattccaaatcagttgatctggggtgagcccagtcctctgtagtttttagaagctcctcctatgtctctcctggtcagcagaatcttggcccctcccttccccccagcctcttggttcttctgggctctgatccagcctcagcgtcactgtcttccacgcccctctttgattctcgtttatgtcaaaagccttgtgaggatgaggctgtgattatccccattttacagatgaggaaactgtggctccaggatgacacaactggccagaggtcacatcagaagcagagctgggtcacttgactccacccaatatccctaaatgcaaacatcccctacagaccgaggctggcaccttagagctggagtccatgcccgctctgaccaggagaagccaacctggtcctccagagccaagagcttctgtccctttcccatctcctgaagcctccctgtcacctttaaagtccattcccacaaagacatcatgggatcaccacagaaaatcaagctctggggctaggctgaccccagctagatttttggctcttttataccccagctgggtggacaagcaccttaaacccgctgagcctcagcttcccgggctataaaatgggggtgatgacacctgcctgtagcattccaaggagggttaaatgtgatgctgcagccaagggtccccacagccaggctctttgcaggtgctgggttcagagtcccagagctgaggccgggagtaggggttcaagtggggtgccccaggcagggtccagtgccagccctctgtggagacagccatccggggccgaggcagccgcccaccgcagggcctgcctatctgcagccagcccagccctcacaaaggaacaataacaggaaaccatcccagggggaagtgggccagggccagctggaaaacctgaaggggaggcagccaggcctccctcgccagcggggtgtggctcccctccaaagacggtcggctgacaggctccacagagctccactcacgctcagccctggacggacaggcagtccaacggaacagaaacatccctcagcccacaggcacggtgagtgggggctcccacactcccctccaccccaaacccgccaccctgcgcccaagatgggagggtcctcagcttccccatctgtagaatgggcatcgtcccactcccatgacagagaggctcc

SEQ ID NO: 21 - DNA, 4956 bp, synthetic construct
  source 1..4956: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 22 - DNA, 4957 bp, synthetic construct
  source 1..4957: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttcctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 23 - DNA, 6100 bp, synthetic construct
  source 1..6100: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 24 - DNA, 6100 bp, synthetic construct
  source 1..6100: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 25 - DNA, 4010 bp, synthetic construct
  source 1..4010: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatga

SEQ ID NO: 26 - DNA, 4010 bp, synthetic construct
  source 1..4010: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatga

SEQ ID NO: 27 - DNA, 8688 bp, synthetic construct
  source 1..8688: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgatcaccaggacccagagctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctgggcagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcaggggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacggcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagtacgtgaacaacagctacgagtgcgacatccccatcggcgccggcatctgcgccagctaccagacccagaccaagagccaccagcaggcccagagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccacaacgcccaggccctgaacaccctggtgaagcagctgagcagcaagttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaatggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 28 - DNA, 10455 bp, synthetic construct
  source 1..10455: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgatcaccaggacccagagctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctgggcagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcaggggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacggcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagtacgtgaacaacagctacgagtgcgacatccccatcggcgccggcatctgcgccagctaccagacccagaccaagagccaccagcaggcccagagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaatggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 29 - DNA, 11599 bp, synthetic construct
  source 1..11599: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgttcgtgttcctggtgctgctgcccctggtgagcagccagtgcgtgaacctgatcaccaggacccagagctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctgggcagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcaggggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacggcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagtacgtgaacaacagctacgagtgcgacatccccatcggcgccggcatctgcgccagctaccagacccagaccaagagccaccagcaggcccagagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccacaacgcccaggccctgaacaccctggtgaagcagctgagcagcaagttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaatggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 30 - DNA, 11599 bp, synthetic construct
  source 1..11599: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgttcgtgttcctggtgctgctgcccctggtgagcagccagtgcgtgaacctgatcaccaggacccagagctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctgggcagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcaggggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacggcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagtacgtgaacaacagctacgagtgcgacatccccatcggcgccggcatctgcgccagctaccagacccagaccaagagccaccagcaggcccagagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaatggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 31 - DNA, 10464 bp, synthetic construct
  source 1..10464: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaatggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 32 - DNA, 10464 bp, synthetic construct
  source 1..10464: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaatggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 33 - DNA, 11608 bp, synthetic construct
  source 1..11608: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaatggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 34 - DNA, 11608 bp, synthetic construct
  source 1..11608: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaatggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 35 - DNA, 10270 bp, synthetic construct
  source 1..10270: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgatcaccaggacccagagctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctgggcagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcaggggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacggcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagtacgtgaacaacagctacgagtgcgacatccccatcggcgccggcatctgcgccagctaccagacccagaccaagagccaccagcaggcccagagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccacaacgcccaggccctgaacaccctggtgaagcagctgagcagcaagttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaattgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 36 - DNA, 10270 bp, synthetic construct
  source 1..10270: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgatcaccaggacccagagctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctgggcagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcaggggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacggcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagtacgtgaacaacagctacgagtgcgacatccccatcggcgccggcatctgcgccagctaccagacccagaccaagagccaccagcaggcccagagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaattgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 37 - DNA, 11414 bp, synthetic construct
  source 1..11414: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgttcgtgttcctggtgctgctgcccctggtgagcagccagtgcgtgaacctgatcaccaggacccagagctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctgggcagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcaggggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacggcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagtacgtgaacaacagctacgagtgcgacatccccatcggcgccggcatctgcgccagctaccagacccagaccaagagccaccagcaggcccagagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccacaacgcccaggccctgaacaccctggtgaagcagctgagcagcaagttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaattgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 38 - DNA, 11414 bp, synthetic construct
  source 1..11414: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgttcgtgttcctggtgctgctgcccctggtgagcagccagtgcgtgaacctgatcaccaggacccagagctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctgggcagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcaggggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacggcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagtacgtgaacaacagctacgagtgcgacatccccatcggcgccggcatctgcgccagctaccagacccagaccaagagccaccagcaggcccagagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaattgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 39 - DNA, 10279 bp, synthetic construct
  source 1..10279: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaattgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 40 - DNA, 10279 bp, synthetic construct
  source 1..10279: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaattgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 41 - DNA, 11423 bp, synthetic construct
  source 1..11423: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaattgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 42 - DNA, 11423 bp, synthetic construct
  source 1..11423: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaattgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 43 - DNA, 9509 bp, synthetic construct
  source 1..9509: mol_type = other DNA
atgttcgtgttcctggtgctgctgcccctggtgagcagccagtgcgtgaacctgatcaccaggacccagagctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctgggcagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcaggggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacggcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagtacgtgaacaacagctacgagtgcgacatccccatcggcgccggcatctgcgccagctaccagacccagaccaagagccaccagcaggcccagagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccacaacgcccaggccctgaacaccctggtgaagcagctgagcagcaagttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaatggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatga

SEQ ID NO: 44 - DNA, 9509 bp, synthetic construct
  source 1..9509: mol_type = other DNA
atgttcgtgttcctggtgctgctgcccctggtgagcagccagtgcgtgaacctgatcaccaggacccagagctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctgggcagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcaggggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacggcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagtacgtgaacaacagctacgagtgcgacatccccatcggcgccggcatctgcgccagctaccagacccagaccaagagccaccagcaggcccagagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaatggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatga

SEQ ID NO: 45 - DNA, 9518 bp, synthetic construct
  source 1..9518: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaatggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatga

SEQ ID NO: 46 - DNA, 9518 bp, synthetic construct
  source 1..9518: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaatggaagcggagccacgaacttctctctgttaaagcaagcaggagatgttgaagaaaaccccgggcctttcggagctattgctggtttcttggaaggaggatgggaaggaatgattgcaggttggcacggatacacatctcatggagcacatggagtagcagtggcagcagaccttaagagtacccatgatga

SEQ ID NO: 47 - DNA, 9324 bp, synthetic construct
  source 1..9324: mol_type = other DNA
atgttcgtgttcctggtgctgctgcccctggtgagcagccagtgcgtgaacctgatcaccaggacccagagctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctgggcagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcaggggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacggcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagtacgtgaacaacagctacgagtgcgacatccccatcggcgccggcatctgcgccagctaccagacccagaccaagagccaccagcaggcccagagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccacaacgcccaggccctgaacaccctggtgaagcagctgagcagcaagttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaattgatga

SEQ ID NO: 48 - DNA, 9324 bp, synthetic construct
  source 1..9324: mol_type = other DNA
atgttcgtgttcctggtgctgctgcccctggtgagcagccagtgcgtgaacctgatcaccaggacccagagctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctgggcagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcaggggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacggcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagtacgtgaacaacagctacgagtgcgacatccccatcggcgccggcatctgcgccagctaccagacccagaccaagagccaccagcaggcccagagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaattgatga

SEQ ID NO: 49 - DNA, 9333 bp, synthetic construct
  source 1..9333: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaattgatga

SEQ ID NO: 50 - DNA, 9333 bp, synthetic construct
  source 1..9333: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacaccggaagcggagccacgaacttctctctgttaaagcaagcaggagatgttgaagaaaaccccgggcctatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaaggaagcggagccacgaacttctctctgttaaagcaagcaggagatgttgaagaaaaccccgggcctagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactggaagcggagccacgaacttctctctgttaaagcaagcaggagatgttgaagaaaaccccgggcctattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaattgatga

SEQ ID NO: 51 - DNA, 1710 bp, synthetic construct
  source 1..1710: mol_type = other DNA
atgatgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaatgatga

SEQ ID NO: 52 - DNA, 1771 bp, synthetic construct
  source 1..1771: mol_type = other DNA
atgagcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctacttgatga

SEQ ID NO: 53 - DNA, 1856 bp, synthetic construct
  source 1..1856: mol_type = other DNA
atgattttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaattgatga

SEQ ID NO: 54 - DNA, 303 bp, synthetic construct
  source 1..303: mol_type = other DNA
atgaacaggaaggtgaccgccatcgccctggccgccatcatctgggccaccgccgcccagggcttcctgatgttcaagcagggcaggtgcctgtgcatcggccccggcatgaaggccgtgaagatggccgagatcgagaaggccagcgtgatctaccccagcaacggctgcgacaaggtggaggtgatcgtgaccatgaaggcccacaagaggcagaggtgcctggaccccaggagcaagcaggccaggctgatcatgcaggccatcgagaagaagaacttcctgaggaggcagaacatgtga

SEQ ID NO: 55 - DNA, 465 bp, synthetic construct
  source 1..465: mol_type = other DNA
atgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtccacaggtcaaggatgccaaagagagagggacagcaagtctggcaggatttcctgtatgactcccggctgaacaagggcaagctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttga

SEQ ID NO: 56 - DNA, 378 bp, synthetic construct
  source 1..378: mol_type = other DNA
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataa

SEQ ID NO: 57 - DNA, 297 bp, synthetic construct
  source 1..297: mol_type = other DNA
atgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaa

SEQ ID NO: 58 - DNA, 300 bp, synthetic construct
  source 1..300: mol_type = other DNA
atgaggctcctggcggccgcgctgctcctgctgctgctggcgctgtacaccgcgcgtgtggacgggtccaaatgcaagtgctcccggaagggacccaagatccgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaagcgcttcatcaagtggtacaacgcctggaacgagaagcgcagggtctacgaagaatag

SEQ ID NO: 59 - DNA, 360 bp, synthetic construct
  source 1..360: mol_type = other DNA
atgaaagttctaatctcttccctcctcctgttgctgccactaatgctgatgtccatggtctctagcagcctgaatccaggggtcgccagaggccacagggaccgaggccaggcttctaggagatggctccaggaaggcggccaagaatgtgagtgcaaagattggttcctgagagccccgagaagaaaattcatgacagtgtctgggctgccaaagaagcagtgcccctgtgatcatttcaagggcaatgtgaagaaaacaagacaccaaaggcaccacagaaagccaaacaagcattccagagcctgccagcaatttctcaaacaatgtcagctaagaagctttgctctgcctttgtag

SEQ ID NO: 60 - DNA, 453 bp, synthetic construct
  source 1..453: mol_type = other DNA
atgaacctgtggctcctggcctgcctggtggccggcttcctgggagcctgggcccccgctgtccacacccaaggtgtctttgaggactgctgcctggcctaccactaccccattgggtgggctgtgctccggcgcgcctggacttaccggatccaggaggtgagcgggagctgcaatctgcctgctgcgatattctacctccccaagagacacaggaaggtgtgtgggaaccccaaaagcagggaggtgcagagagccatgaagctcctggatgctcgaaataaggtttttgcaaagctccaccacaacacgcagaccttccaagcaggccctcatgctgtaaagaagttgagttctggaaactccaagttatcatcgtccaagtttagcaatcccatcagcagcagtaagaggaatgtctccctcctgatatcagctaattcaggactgtga

SEQ ID NO: 61 - DNA, 384 bp, synthetic construct
  source 1..384: mol_type = other DNA
atgcagcagagaggactcgccatcgtggccttggctgtctgtgcggccctacatgcctcagaagccatacttcccattgcctccagctgttgcacggaggtttcacatcatatttccagaaggctcctggaaagagtgaatatgtgtcgcatccagagagctgatggggattgtgacttggctgctgtcatccttcatgtcaagcgcagaagaatctgtgtcagcccgcacaaccatactgttaagcagtggatgaaagtgcaagctgccaagaaaaatggtaaaggaaatgtttgccacaggaagaaacaccatggcaagaggaacagtaacagggcacatcaggggaaacacgaaacatacggccataaaactccttattag

SEQ ID NO: 62 - DNA, 465 bp, synthetic construct
  source 1..465: mol_type = other DNA
atgttccacgtgagcttcaggtacatcttcggcatcccccccctgatcctggtgctgctgcccgtgaccagcagcgagtgccacatcaaggacaaggagggcaaggcctacgagagcgtgctgatgatcagcatcgacgagctggacaagatgaccggcaccgacagcaactgccccaacaacgagcccaacttcttcaggaagcacgtgtgcgacgacaccaaggaggccgccttcctgaacagggccgccaggaagctgaagcagttcctgaagatgaacatcagcgaggagttcaacgtgcacctgctgaccgtgagccagggcacccagaccctggtgaactgcaccagcaaggaggagaagaacgtgaaggagcagaagaagaacgacgcctgcttcctgaagaggctgctgagggagatcaagacctgctggaacaagatcctgaagggcagcatctga

SEQ ID NO: 63 - DNA, 489 bp, synthetic construct
  source 1..489: mol_type = other DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 64 - DNA, 462 bp, synthetic construct
  source 1..462: mol_type = other DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 65 - DNA, 63 bp, synthetic construct
  source 1..63: mol_type = other DNA
ggaagcggagagggcaggggaagtcttctaacatgcggggacgtggaggaaaatcccggcccc

SEQ ID NO: 66 - DNA, 69 bp, synthetic construct
  source 1..69: mol_type = other DNA
ggaagcggacagtgtactaattatgctctcttgaaattggctggagatgttgagagcaacccaggtccc

SEQ ID NO: 67 - DNA, 66 bp, synthetic construct
  source 1..66: mol_type = other DNA
ggaagcggagccacgaacttctctctgttaaagcaagcaggagatgttgaagaaaaccccgggcct

SEQ ID NO: 68 - DNA, 15 bp, synthetic construct
  source 1..15: mol_type = other DNA
ggccccggccccggc

SEQ ID NO: 69 - DNA, 18 bp, synthetic construct
  source 1..18: mol_type = other DNA
catcaccatcaccatcac